SEQUENCE LISTING (1102 sequences)

SEQ ID NO: 1 - protein, 19 aa, synthetic construct
  REGION 1..19: note = an artificially synthesised sequence
MGWSCIILFLVATATGVHS

SEQ ID NO: 2 - protein, 8 aa, synthetic construct
  REGION 1..8: note = an artificially synthesised sequence
LSGRSDNH

SEQ ID NO: 3 - protein, 6 aa, synthetic construct
  REGION 1..6: note = an artificially synthesised sequence
PLGLAG

SEQ ID NO: 4 - protein, 8 aa, synthetic construct
  REGION 1..8: note = an artificially synthesised sequence
VPLSLTMG

SEQ ID NO: 5 - protein, 12 aa, synthetic construct
  REGION 1..12: note = an artificially synthesised sequence
TSTSGRSANPRG

SEQ ID NO: 6 - protein, 13 aa, synthetic construct
  REGION 1..13: note = an artificially synthesised sequence
ISSGLLSGRSDNH

SEQ ID NO: 7 - protein, 18 aa, synthetic construct
  REGION 1..18: note = an artificially synthesised sequence
AVGLLAPPGGLSGRSDNH

SEQ ID NO: 8 - protein, 13 aa, synthetic construct
  REGION 1..13: note = an artificially synthesised sequence
GAGVPMSMRGGAG

SEQ ID NO: 9 - protein, 13 aa, synthetic construct
  REGION 1..13: note = an artificially synthesised sequence
GAGIPVSLRSGAG

SEQ ID NO: 10 - protein, 8 aa, synthetic construct
  REGION 1..8: note = an artificially synthesised sequence
GPLGIAGQ

SEQ ID NO: 11 - protein, 10 aa, synthetic construct
  REGION 1..10: note = an artificially synthesised sequence
GGPLGMLSQS

SEQ ID NO: 12 - protein, 6 aa, synthetic construct
  REGION 1..6: note = an artificially synthesised sequence
PLGLWA

SEQ ID NO: 13 - protein, 13 aa, synthetic construct
  REGION 1..13: note = an artificially synthesised sequence
GAGRPFSMIMGAG

SEQ ID NO: 14 - protein, 13 aa, synthetic construct
  REGION 1..13: note = an artificially synthesised sequence
GAGVPLSLTMGAG

SEQ ID NO: 15 - protein, 13 aa, synthetic construct
  REGION 1..13: note = an artificially synthesised sequence
GAGVPLSLYSGAG

SEQ ID NO: 16 - protein, 6 aa, synthetic construct
  REGION 1..6: note = an artificially synthesised sequence
AANLRN

SEQ ID NO: 17 - protein, 6 aa, synthetic construct
  REGION 1..6: note = an artificially synthesised sequence
AQAYVK

SEQ ID NO: 18 - protein, 6 aa, synthetic construct
  REGION 1..6: note = an artificially synthesised sequence
AANYMR

SEQ ID NO: 19 - protein, 6 aa, synthetic construct
  REGION 1..6: note = an artificially synthesised sequence
AAALTR

SEQ ID NO: 20 - protein, 6 aa, synthetic construct
  REGION 1..6: note = an artificially synthesised sequence
AQNLMR

SEQ ID NO: 21 - protein, 6 aa, synthetic construct
  REGION 1..6: note = an artificially synthesised sequence
AANYTK

SEQ ID NO: 22 - protein, 16 aa, synthetic construct
  REGION 1..16: note = an artificially synthesised sequence
GAGPQGLAGQRGIVAG

SEQ ID NO: 23 - protein, 8 aa, synthetic construct
  REGION 1..8: note = an artificially synthesised sequence
PRFKIIGG

SEQ ID NO: 24 - protein, 8 aa, synthetic construct
  REGION 1..8: note = an artificially synthesised sequence
PRFRIIGG

SEQ ID NO: 25 - protein, 9 aa, synthetic construct
  REGION 1..9: note = an artificially synthesised sequence
GAGSGRSAG

SEQ ID NO: 26 - protein, 5 aa, synthetic construct
  REGION 1..5: note = an artificially synthesised sequence
SGRSA

SEQ ID NO: 27 - protein, 6 aa, synthetic construct
  REGION 1..6: note = an artificially synthesised sequence
GSGRSA

SEQ ID NO: 28 - protein, 5 aa, synthetic construct
  REGION 1..5: note = an artificially synthesised sequence
SGKSA

SEQ ID NO: 29 - protein, 5 aa, synthetic construct
  REGION 1..5: note = an artificially synthesised sequence
SGRSS

SEQ ID NO: 30 - protein, 5 aa, synthetic construct
  REGION 1..5: note = an artificially synthesised sequence
SGRRA

SEQ ID NO: 31 - protein, 5 aa, synthetic construct
  REGION 1..5: note = an artificially synthesised sequence
SGRNA

SEQ ID NO: 32 - protein, 5 aa, synthetic construct
  REGION 1..5: note = an artificially synthesised sequence
SGRKA

SEQ ID NO: 33 - protein, 6 aa, synthetic construct
  REGION 1..6: note = an artificially synthesised sequence
QRGRSA

SEQ ID NO: 34 - protein, 17 aa, synthetic construct
  REGION 1..17: note = an artificially synthesised sequence
GAGSLLKSRMVPNFNAG

SEQ ID NO: 35 - protein, 6 aa, synthetic construct
  REGION 1..6: note = an artificially synthesised sequence
TQGAAA

SEQ ID NO: 36 - protein, 6 aa, synthetic construct
  REGION 1..6: note = an artificially synthesised sequence
GAAAAA

SEQ ID NO: 37 - protein, 6 aa, synthetic construct
  REGION 1..6: note = an artificially synthesised sequence
GAGAAG

SEQ ID NO: 38 - protein, 6 aa, synthetic construct
  REGION 1..6: note = an artificially synthesised sequence
AAAAAG

SEQ ID NO: 39 - protein, 6 aa, synthetic construct
  REGION 1..6: note = an artificially synthesised sequence
LCGAAI

SEQ ID NO: 40 - protein, 6 aa, synthetic construct
  REGION 1..6: note = an artificially synthesised sequence
FAQALG

SEQ ID NO: 41 - protein, 6 aa, synthetic construct
  REGION 1..6: note = an artificially synthesised sequence
LLQANP

SEQ ID NO: 42 - protein, 6 aa, synthetic construct
  REGION 1..6: note = an artificially synthesised sequence
LAAANP

SEQ ID NO: 43 - protein, 6 aa, synthetic construct
  REGION 1..6: note = an artificially synthesised sequence
LYGAQF

SEQ ID NO: 44 - protein, 6 aa, synthetic construct
  REGION 1..6: note = an artificially synthesised sequence
LSQAQG

SEQ ID NO: 45 - protein, 6 aa, synthetic construct
  REGION 1..6: note = an artificially synthesised sequence
ASAASG

SEQ ID NO: 46 - protein, 6 aa, synthetic construct
  REGION 1..6: note = an artificially synthesised sequence
FLGASL

SEQ ID NO: 47 - protein, 6 aa, synthetic construct
  REGION 1..6: note = an artificially synthesised sequence
AYGATG

SEQ ID NO: 48 - protein, 6 aa, synthetic construct
  REGION 1..6: note = an artificially synthesised sequence
LAQATG

SEQ ID NO: 49 - protein, 17 aa, synthetic construct
  REGION 1..17: note = an artificially synthesised sequence
GAGSGVVIATVIVITAG

SEQ ID NO: 50 - protein, 8 aa, synthetic construct
  REGION 1..8: note = an artificially synthesised sequence
APMAEGGG

SEQ ID NO: 51 - protein, 8 aa, synthetic construct
  REGION 1..8: note = an artificially synthesised sequence
EAQGDKII

SEQ ID NO: 52 - protein, 8 aa, synthetic construct
  REGION 1..8: note = an artificially synthesised sequence
LAFSDAGP

SEQ ID NO: 53 - protein, 7 aa, synthetic construct
  REGION 1..7: note = an artificially synthesised sequence
YVADAPK

SEQ ID NO: 54 - protein, 5 aa, synthetic construct
  REGION 1..5: note = an artificially synthesised sequence
RRRRR

SEQ ID NO: 55 - protein, 6 aa, synthetic construct
  REGION 1..6: note = an artificially synthesised sequence
RRRRRR

SEQ ID NO: 56 - protein, 10 aa, synthetic construct
  REGION 1..10: note = an artificially synthesised sequence
GQSSRHRRAL

SEQ ID NO: 57 - protein, 9 aa, synthetic construct
  REGION 1..9: note = an artificially synthesised sequence
SSRHRRALD

SEQ ID NO: 58 - protein, 14 aa, synthetic construct
  REGION 1..14: note = an artificially synthesised sequence
RKSSIIIRMRDVVL

SEQ ID NO: 59 - protein, 15 aa, synthetic construct
  REGION 1..15: note = an artificially synthesised sequence
SSSFDKGKYKKGDDA

SEQ ID NO: 60 - protein, 15 aa, synthetic construct
  REGION 1..15: note = an artificially synthesised sequence
SSSFDKGKYKRGDDA

SEQ ID NO: 61 - protein, 4 aa, synthetic construct
  REGION 1..4: note = an artificially synthesised sequence
IEGR

SEQ ID NO: 62 - protein, 4 aa, synthetic construct
  REGION 1..4: note = an artificially synthesised sequence
IDGR

SEQ ID NO: 63 - protein, 7 aa, synthetic construct
  REGION 1..7: note = an artificially synthesised sequence
GGSIDGR

SEQ ID NO: 64 - protein, 8 aa, synthetic construct
  REGION 1..8: note = an artificially synthesised sequence
GPQGIAGQ

SEQ ID NO: 65 - protein, 8 aa, synthetic construct
  REGION 1..8: note = an artificially synthesised sequence
GPQGLLGA

SEQ ID NO: 66 - protein, 5 aa, synthetic construct
  REGION 1..5: note = an artificially synthesised sequence
GIAGQ

SEQ ID NO: 67 - protein, 7 aa, synthetic construct
  REGION 1..7: note = an artificially synthesised sequence
GPLGIAG

SEQ ID NO: 68 - protein, 8 aa, synthetic construct
  REGION 1..8: note = an artificially synthesised sequence
GPEGLRVG

SEQ ID NO: 69 - protein, 8 aa, synthetic construct
  REGION 1..8: note = an artificially synthesised sequence
YGAGLGVV

SEQ ID NO: 70 - protein, 8 aa, synthetic construct
  REGION 1..8: note = an artificially synthesised sequence
AGLGVVER

SEQ ID NO: 71 - protein, 8 aa, synthetic construct
  REGION 1..8: note = an artificially synthesised sequence
AGLGISST

SEQ ID NO: 72 - protein, 8 aa, synthetic construct
  REGION 1..8: note = an artificially synthesised sequence
EPQALAMS

SEQ ID NO: 73 - protein, 8 aa, synthetic construct
  REGION 1..8: note = an artificially synthesised sequence
QALAMSAI

SEQ ID NO: 74 - protein, 8 aa, synthetic construct
  REGION 1..8: note = an artificially synthesised sequence
AAYHLVSQ

SEQ ID NO: 75 - protein, 8 aa, synthetic construct
  REGION 1..8: note = an artificially synthesised sequence
MDAFLESS

SEQ ID NO: 76 - protein, 8 aa, synthetic construct
  REGION 1..8: note = an artificially synthesised sequence
ESLPVVAV

SEQ ID NO: 77 - protein, 8 aa, synthetic construct
  REGION 1..8: note = an artificially synthesised sequence
SAPAVESE

SEQ ID NO: 78 - protein, 8 aa, synthetic construct
  REGION 1..8: note = an artificially synthesised sequence
DVAQFVLT

SEQ ID NO: 79 - protein, 8 aa, synthetic construct
  REGION 1..8: note = an artificially synthesised sequence
VAQFVLTE

SEQ ID NO: 80 - protein, 8 aa, synthetic construct
  REGION 1..8: note = an artificially synthesised sequence
AQFVLTEG

SEQ ID NO: 81 - protein, 8 aa, synthetic construct
  REGION 1..8: note = an artificially synthesised sequence
PVQPIGPQ

SEQ ID NO: 82 - protein, 6 aa, synthetic construct
  REGION 1..6: note = an artificially synthesised sequence
LVPRGS

SEQ ID NO: 83 - protein, 12 aa, synthetic construct
  REGION 1..12: note = an artificially synthesised sequence
TSTSGRSANPRG

SEQ ID NO: 84 - protein, 12 aa, synthetic construct
  REGION 1..12: note = an artificially synthesised sequence
TSTSGRSANPRG

SEQ ID NO: 85 - protein, 12 aa, synthetic construct
  REGION 1..12: note = an artificially synthesised sequence
TSGSGRSANARG

SEQ ID NO: 86 - protein, 12 aa, synthetic construct
  REGION 1..12: note = an artificially synthesised sequence
TSQSGRSANQRG

SEQ ID NO: 87 - protein, 12 aa, synthetic construct
  REGION 1..12: note = an artificially synthesised sequence
TSPSGRSAYPRG

SEQ ID NO: 88 - protein, 12 aa, synthetic construct
  REGION 1..12: note = an artificially synthesised sequence
TSGSGRSATPRG

SEQ ID NO: 89 - protein, 12 aa, synthetic construct
  REGION 1..12: note = an artificially synthesised sequence
TSQSGRSATPRG

SEQ ID NO: 90 - protein, 12 aa, synthetic construct
  REGION 1..12: note = an artificially synthesised sequence
TSASGRSATPRG

SEQ ID NO: 91 - protein, 12 aa, synthetic construct
  REGION 1..12: note = an artificially synthesised sequence
TSYSGRSAVPRG

SEQ ID NO: 92 - protein, 12 aa, synthetic construct
  REGION 1..12: note = an artificially synthesised sequence
TSYSGRSANFRG

SEQ ID NO: 93 - protein, 12 aa, synthetic construct
  REGION 1..12: note = an artificially synthesised sequence
TSSSGRSATPRG

SEQ ID NO: 94 - protein, 12 aa, synthetic construct
  REGION 1..12: note = an artificially synthesised sequence
TSTTGRSASPRG

SEQ ID NO: 95 - protein, 12 aa, synthetic construct
  REGION 1..12: note = an artificially synthesised sequence
TSTSGRSANPRG

SEQ ID NO: 96-135 - skipped (40 entries)

SEQ ID NO: 136 - protein, 4 aa, synthetic construct
  REGION 1..4: note = an artificially synthesised sequence
GGGS

SEQ ID NO: 137 - protein, 4 aa, synthetic construct
  REGION 1..4: note = an artificially synthesised sequence
GGSG

SEQ ID NO: 138 - protein, 4 aa, synthetic construct
  REGION 1..4: note = an artificially synthesised sequence
GSGG

SEQ ID NO: 139 - protein, 4 aa, synthetic construct
  REGION 1..4: note = an artificially synthesised sequence
SGGG

SEQ ID NO: 140 - protein, 4 aa, synthetic construct
  REGION 1..4: note = an artificially synthesised sequence
GSSG

SEQ ID NO: 141 - protein, 5 aa, synthetic construct
  REGION 1..5: note = an artificially synthesised sequence
GGGGS

SEQ ID NO: 142 - protein, 5 aa, synthetic construct
  REGION 1..5: note = an artificially synthesised sequence
GGGSG

SEQ ID NO: 143 - protein, 5 aa, synthetic construct
  REGION 1..5: note = an artificially synthesised sequence
GGSGG

SEQ ID NO: 144 - protein, 5 aa, synthetic construct
  REGION 1..5: note = an artificially synthesised sequence
GSGGG

SEQ ID NO: 145 - protein, 5 aa, synthetic construct
  REGION 1..5: note = an artificially synthesised sequence
GSGGS

SEQ ID NO: 146 - protein, 5 aa, synthetic construct
  REGION 1..5: note = an artificially synthesised sequence
SGGGG

SEQ ID NO: 147 - protein, 5 aa, synthetic construct
  REGION 1..5: note = an artificially synthesised sequence
GSSGG

SEQ ID NO: 148 - protein, 5 aa, synthetic construct
  REGION 1..5: note = an artificially synthesised sequence
GSGSG

SEQ ID NO: 149 - protein, 5 aa, synthetic construct
  REGION 1..5: note = an artificially synthesised sequence
SGGSG

SEQ ID NO: 150 - protein, 5 aa, synthetic construct
  REGION 1..5: note = an artificially synthesised sequence
GSSSG

SEQ ID NO: 151 - protein, 6 aa, synthetic construct
  REGION 1..6: note = an artificially synthesised sequence
GGGGGS

SEQ ID NO: 152 - protein, 6 aa, synthetic construct
  REGION 1..6: note = an artificially synthesised sequence
SGGGGG

SEQ ID NO: 153 - protein, 7 aa, synthetic construct
  REGION 1..7: note = an artificially synthesised sequence
GGGGGGS

SEQ ID NO: 154 - protein, 7 aa, synthetic construct
  REGION 1..7: note = an artificially synthesised sequence
SGGGGGG

SEQ ID NO: 155 - protein, 330 aa, synthetic construct [sequence omitted]

SEQ ID NO: 156 - protein, 326 aa, synthetic construct
  REGION 1..326: note = an artificially synthesised sequence
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 157 - protein, 377 aa, synthetic construct
  REGION 1..377: note = an artificially synthesised sequence
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 158 - protein, 327 aa, synthetic construct
  REGION 1..327: note = an artificially synthesised sequence
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 159 - protein, 6 aa, synthetic construct
  REGION 1..6: note = an artificially synthesised sequence
MYPPPY

SEQ ID NO: 160 - protein, 12 aa, synthetic construct
  REGION 1..12: note = an artificially synthesised sequence
TSASGRSANPRG

SEQ ID NO: 161 - protein, 12 aa, synthetic construct
  REGION 1..12: note = an artificially synthesised sequence
TSESGRSANPRG

SEQ ID NO: 162 - protein, 12 aa, synthetic construct
  REGION 1..12: note = an artificially synthesised sequence
TSFSGRSANPRG

SEQ ID NO: 163 - protein, 12 aa, synthetic construct
  REGION 1..12: note = an artificially synthesised sequence
TSGSGRSANPRG

SEQ ID NO: 164 - protein, 12 aa, synthetic construct
  REGION 1..12: note = an artificially synthesised sequence
TSHSGRSANPRG

SEQ ID NO: 165 - protein, 12 aa, synthetic construct
  REGION 1..12: note = an artificially synthesised sequence
TSKSGRSANPRG

SEQ ID NO: 166 - protein, 12 aa, synthetic construct
  REGION 1..12: note = an artificially synthesised sequence
TSMSGRSANPRG

SEQ ID NO: 167 - protein, 12 aa, synthetic construct
  REGION 1..12: note = an artificially synthesised sequence
TSNSGRSANPRG

SEQ ID NO: 168 - protein, 12 aa, synthetic construct
  REGION 1..12: note = an artificially synthesised sequence
TSPSGRSANPRG

SEQ ID NO: 169 - protein, 12 aa, synthetic construct
  REGION 1..12: note = an artificially synthesised sequence
TSQSGRSANPRG

SEQ ID NO: 170 - protein, 12 aa, synthetic construct
  REGION 1..12: note = an artificially synthesised sequence
TSWSGRSANPRG

SEQ ID NO: 171 - protein, 12 aa, synthetic construct
  REGION 1..12: note = an artificially synthesised sequence
TSYSGRSANPRG

SEQ ID NO: 172 - protein, 12 aa, synthetic construct
  REGION 1..12: note = an artificially synthesised sequence
TSTAGRSANPRG

SEQ ID NO: 173 - protein, 12 aa, synthetic construct
  REGION 1..12: note = an artificially synthesised sequence
TSTDGRSANPRG

SEQ ID NO: 174 - protein, 12 aa, synthetic construct
  REGION 1..12: note = an artificially synthesised sequence
TSTEGRSANPRG

SEQ ID NO: 175 - protein, 12 aa, synthetic construct
  REGION 1..12: note = an artificially synthesised sequence
TSTFGRSANPRG

SEQ ID NO: 176 - protein, 12 aa, synthetic construct
  REGION 1..12: note = an artificially synthesised sequence
TSTLGRSANPRG

SEQ ID NO: 177 - protein, 12 aa, synthetic construct
  REGION 1..12: note = an artificially synthesised sequence
TSTMGRSANPRG

SEQ ID NO: 178 - protein, 12 aa, synthetic construct
  REGION 1..12: note = an artificially synthesised sequence
TSTPGRSANPRG

SEQ ID NO: 179 - protein, 12 aa, synthetic construct
  REGION 1..12: note = an artificially synthesised sequence
TSTQGRSANPRG

SEQ ID NO: 180 - protein, 12 aa, synthetic construct
  REGION 1..12: note = an artificially synthesised sequence
TSTVGRSANPRG

SEQ ID NO: 181 - protein, 12 aa, synthetic construct
  REGION 1..12: note = an artificially synthesised sequence
TSTWGRSANPRG

SEQ ID NO: 182 - protein, 12 aa, synthetic construct
  REGION 1..12: note = an artificially synthesised sequence
TSTSARSANPRG

SEQ ID NO: 183 - protein, 12 aa, synthetic construct
  REGION 1..12: note = an artificially synthesised sequence
TSTSERSANPRG

SEQ ID NO: 184 - protein, 12 aa, synthetic construct
  REGION 1..12: note = an artificially synthesised sequence
TSTSFRSANPRG

SEQ ID NO: 185 - protein, 12 aa, synthetic construct
  REGION 1..12: note = an artificially synthesised sequence
TSTSHRSANPRG

SEQ ID NO: 186 - protein, 12 aa, synthetic construct
  REGION 1..12: note = an artificially synthesised sequence
TSTSIRSANPRG

SEQ ID NO: 187 - protein, 12 aa, synthetic construct
  REGION 1..12: note = an artificially synthesised sequence
TSTSKRSANPRG

SEQ ID NO: 188 - protein, 12 aa, synthetic construct
  REGION 1..12: note = an artificially synthesised sequence
TSTSLRSANPRG

SEQ ID NO: 189 - protein, 12 aa, synthetic construct
  REGION 1..12: note = an artificially synthesised sequence
TSTSMRSANPRG

SEQ ID NO: 190 - protein, 12 aa, synthetic construct
  REGION 1..12: note = an artificially synthesised sequence
TSTSNRSANPRG

SEQ ID NO: 191 - protein, 12 aa, synthetic construct
  REGION 1..12: note = an artificially synthesised sequence
TSTSPRSANPRG

SEQ ID NO: 192 - protein, 12 aa, synthetic construct
  REGION 1..12: note = an artificially synthesised sequence
TSTSQRSANPRG

SEQ ID NO: 193 - protein, 12 aa, synthetic construct
  REGION 1..12: note = an artificially synthesised sequence
TSTSRRSANPRG

SEQ ID NO: 194 - protein, 12 aa, synthetic construct
  REGION 1..12: note = an artificially synthesised sequence
TSTSTRSANPRG

SEQ ID NO: 195 - protein, 12 aa, synthetic construct
  REGION 1..12: note = an artificially synthesised sequence
TSTSVRSANPRG

SEQ ID NO: 196 - protein, 12 aa, synthetic construct
  REGION 1..12: note = an artificially synthesised sequence
TSTSWRSANPRG

SEQ ID NO: 197 - protein, 12 aa, synthetic construct
  REGION 1..12: note = an artificially synthesised sequence
TSTSYRSANPRG

SEQ ID NO: 198 - protein, 12 aa, synthetic construct
  REGION 1..12: note = an artificially synthesised sequence
TSTSGRAANPRG

SEQ ID NO: 199 - protein, 12 aa, synthetic construct
  REGION 1..12: note = an artificially synthesised sequence
TSTSGRDANPRG

SEQ ID NO: 200 - protein, 12 aa, synthetic construct
  REGION 1..12: note = an artificially synthesised sequence
TSTSGREANPRG

SEQ ID NO: 201 - protein, 12 aa, synthetic construct
  REGION 1..12: note = an artificially synthesised sequence
TSTSGRGANPRG

SEQ ID NO: 202 - protein, 12 aa, synthetic construct
  REGION 1..12: note = an artificially synthesised sequence
TSTSGRHANPRG

SEQ ID NO: 203 - protein, 12 aa, synthetic construct
  REGION 1..12: note = an artificially synthesised sequence
TSTSGRIANPRG

SEQ ID NO: 204 - protein, 12 aa, synthetic construct
  REGION 1..12: note = an artificially synthesised sequence
TSTSGRKANPRG

SEQ ID NO: 205 - protein, 12 aa, synthetic construct
  REGION 1..12: note = an artificially synthesised sequence
TSTSGRLANPRG

SEQ ID NO: 206 - protein, 12 aa, synthetic construct
  REGION 1..12: note = an artificially synthesised sequence
TSTSGRMANPRG

SEQ ID NO: 207 - protein, 12 aa, synthetic construct
  REGION 1..12: note = an artificially synthesised sequence
TSTSGRNANPRG

SEQ ID NO: 208 - protein, 12 aa, synthetic construct
  REGION 1..12: note = an artificially synthesised sequence
TSTSGRPANPRG

SEQ ID NO: 209 - protein, 12 aa, synthetic construct
  REGION 1..12: note = an artificially synthesised sequence
TSTSGRQANPRG

SEQ ID NO: 210 - protein, 12 aa, synthetic construct
  REGION 1..12: note = an artificially synthesised sequence
TSTSGRRANPRG

SEQ ID NO: 211 - protein, 12 aa, synthetic construct
  REGION 1..12: note = an artificially synthesised sequence
TSTSGRTANPRG

SEQ ID NO: 212 - protein, 12 aa, synthetic construct
  REGION 1..12: note = an artificially synthesised sequence
TSTSGRVANPRG

SEQ ID NO: 213 - protein, 12 aa, synthetic construct
  REGION 1..12: note = an artificially synthesised sequence
TSTSGRWANPRG

SEQ ID NO: 214 - protein, 12 aa, synthetic construct
  REGION 1..12: note = an artificially synthesised sequence
TSTSGRYANPRG

SEQ ID NO: 215 - protein, 12 aa, synthetic construct
  REGION 1..12: note = an artificially synthesised sequence
TSTSGRSENPRG

SEQ ID NO: 216 - protein, 12 aa, synthetic construct
  REGION 1..12: note = an artificially synthesised sequence
TSTSGRSFNPRG

SEQ ID NO: 217 - protein, 12 aa, synthetic construct
  REGION 1..12: note = an artificially synthesised sequence
TSTSGRSKNPRG

SEQ ID NO: 218 - protein, 12 aa, synthetic construct
  REGION 1..12: note = an artificially synthesised sequence
TSTSGRSMNPRG

SEQ ID NO: 219 - protein, 12 aa, synthetic construct
  REGION 1..12: note = an artificially synthesised sequence
TSTSGRSNNPRG

SEQ ID NO: 220 - protein, 12 aa, synthetic construct
  REGION 1..12: note = an artificially synthesised sequence
TSTSGRSPNPRG

SEQ ID NO: 221 - protein, 12 aa, synthetic construct
  REGION 1..12: note = an artificially synthesised sequence
TSTSGRSQNPRG

SEQ ID NO: 222 - protein, 12 aa, synthetic construct
  REGION 1..12: note = an artificially synthesised sequence
TSTSGRSRNPRG

SEQ ID NO: 223 - protein, 12 aa, synthetic construct
  REGION 1..12: note = an artificially synthesised sequence
TSTSGRSSNPRG

SEQ ID NO: 224 - protein, 12 aa, synthetic construct
  REGION 1..12: note = an artificially synthesised sequence
TSTSGRSWNPRG

SEQ ID NO: 225 - protein, 12 aa, synthetic construct
  REGION 1..12: note = an artificially synthesised sequence
TSTSGRSYNPRG

SEQ ID NO: 226 - protein, 12 aa, synthetic construct
  REGION 1..12: note = an artificially synthesised sequence
TSTSGRSAAPRG

SEQ ID NO: 227 - protein, 12 aa, synthetic construct
  REGION 1..12: note = an artificially synthesised sequence
TSTSGRSADPRG

SEQ ID NO: 228 - protein, 12 aa, synthetic construct
  REGION 1..12: note = an artificially synthesised sequence
TSTSGRSAEPRG

SEQ ID NO: 229 - protein, 12 aa, synthetic construct
  REGION 1..12: note = an artificially synthesised sequence
TSTSGRSAFPRG

SEQ ID NO: 230 - protein, 12 aa, synthetic construct
  REGION 1..12: note = an artificially synthesised sequence
TSTSGRSAGPRG

SEQ ID NO: 231 - protein, 12 aa, synthetic construct
  REGION 1..12: note = an artificially synthesised sequence
TSTSGRSAKPRG

SEQ ID NO: 232 - protein, 12 aa, synthetic construct
  REGION 1..12: note = an artificially synthesised sequence
TSTSGRSALPRG

SEQ ID NO: 233 - protein, 12 aa, synthetic construct
  REGION 1..12: note = an artificially synthesised sequence
TSTSGRSAMPRG

SEQ ID NO: 234 - protein, 12 aa, synthetic construct
  REGION 1..12: note = an artificially synthesised sequence
TSTSGRSAPPRG

SEQ ID NO: 235 - protein, 12 aa, synthetic construct
  REGION 1..12: note = an artificially synthesised sequence
TSTSGRSAQPRG

SEQ ID NO: 236 - protein, 12 aa, synthetic construct
  REGION 1..12: note = an artificially synthesised sequence
TSTSGRSAVPRG

SEQ ID NO: 237 - protein, 12 aa, synthetic construct
  REGION 1..12: note = an artificially synthesised sequence
TSTSGRSAWPRG

SEQ ID NO: 238 - protein, 12 aa, synthetic construct
  REGION 1..12: note = an artificially synthesised sequence
TSTSGRSAYPRG

SEQ ID NO: 239 - protein, 12 aa, synthetic construct
  REGION 1..12: note = an artificially synthesised sequence
TSTSGRSANARG

SEQ ID NO: 240 - protein, 12 aa, synthetic construct
  REGION 1..12: note = an artificially synthesised sequence
TSTSGRSANDRG

SEQ ID NO: 241 - protein, 12 aa, synthetic construct
  REGION 1..12: note = an artificially synthesised sequence
TSTSGRSANERG

SEQ ID NO: 242 - protein, 12 aa, synthetic construct
  REGION 1..12: note = an artificially synthesised sequence
TSTSGRSANFRG

SEQ ID NO: 243 - protein, 12 aa, synthetic construct
  REGION 1..12: note = an artificially synthesised sequence
TSTSGRSANGRG

SEQ ID NO: 244 - protein, 12 aa, synthetic construct
  REGION 1..12: note = an artificially synthesised sequence
TSTSGRSANIRG

SEQ ID NO: 245 - protein, 12 aa, synthetic construct
  REGION 1..12: note = an artificially synthesised sequence
TSTSGRSANKRG

SEQ ID NO: 246 - protein, 12 aa, synthetic construct
  REGION 1..12: note = an artificially synthesised sequence
TSTSGRSANNRG

SEQ ID NO: 247 - protein, 12 aa, synthetic construct
  REGION 1..12: note = an artificially synthesised sequence
TSTSGRSANQRG

SEQ ID NO: 248 - protein, 12 aa, synthetic construct
  REGION 1..12: note = an artificially synthesised sequence
TSTSGRSANSRG

SEQ ID NO: 249 - protein, 12 aa, synthetic construct
  REGION 1..12: note = an artificially synthesised sequence
TSTSGRSANTRG

SEQ ID NO: 250 - protein, 12 aa, synthetic construct
  REGION 1..12: note = an artificially synthesised sequence
TSTSGRSANWRG

SEQ ID NO: 251 - protein, 12 aa, synthetic construct
  REGION 1..12: note = an artificially synthesised sequence
TSDSGRSANPRG

SEQ ID NO: 252 - protein, 12 aa, synthetic construct
  REGION 1..12: note = an artificially synthesised sequence
TSISGRSANPRG

SEQ ID NO: 253 - protein, 12 aa, synthetic construct
  REGION 1..12: note = an artificially synthesised sequence
TSSSGRSANPRG

SEQ ID NO: 254 - protein, 12 aa, synthetic construct
  REGION 1..12: note = an artificially synthesised sequence
TSTHGRSANPRG

SEQ ID NO: 255 - protein, 12 aa, synthetic construct
  REGION 1..12: note = an artificially synthesised sequence
TSTKGRSANPRG

SEQ ID NO: 256 - protein, 12 aa, synthetic construct
  REGION 1..12: note = an artificially synthesised sequence
TSTTGRSANPRG

SEQ ID NO: 257 - protein, 12 aa, synthetic construct
  REGION 1..12: note = an artificially synthesised sequence
TSTYGRSANPRG

SEQ ID NO: 258 - protein, 12 aa, synthetic construct
  REGION 1..12: note = an artificially synthesised sequence
TSTSDRSANPRG

SEQ ID NO: 259 - protein, 12 aa, synthetic construct
  REGION 1..12: note = an artificially synthesised sequence
TSTSSRSANPRG

SEQ ID NO: 260 - protein, 12 aa, synthetic construct
  REGION 1..12: note = an artificially synthesised sequence
TSTSGRFANPRG

SEQ ID NO: 261 - protein, 12 aa, synthetic construct
  REGION 1..12: note = an artificially synthesised sequence
TSTSGRSDNPRG

SEQ ID NO: 262 - protein, 12 aa, synthetic construct
  REGION 1..12: note = an artificially synthesised sequence
TSTSGRSHNPRG

SEQ ID NO: 263 - protein, 12 aa, synthetic construct
  REGION 1..12: note = an artificially synthesised sequence
TSTSGRSINPRG

SEQ ID NO: 264 - protein, 12 aa, synthetic construct
  REGION 1..12: note = an artificially synthesised sequence
TSTSGRSLNPRG

SEQ ID NO: 265 - protein, 12 aa, synthetic construct
  REGION 1..12: note = an artificially synthesised sequence
TSTSGRSTNPRG

SEQ ID NO: 266 - protein, 12 aa, synthetic construct
  REGION 1..12: note = an artificially synthesised sequence
TSTSGRSVNPRG

SEQ ID NO: 267 - protein, 12 aa, synthetic construct
  REGION 1..12: note = an artificially synthesised sequence
TSTSGRSAHPRG

SEQ ID NO: 268 - protein, 12 aa, synthetic construct
  REGION 1..12: note = an artificially synthesised sequence
TSTSGRSAIPRG

SEQ ID NO: 269 - protein, 12 aa, synthetic construct
  REGION 1..12: note = an artificially synthesised sequence
TSTSGRSARPRG

SEQ ID NO: 270 - protein, 12 aa, synthetic construct
  REGION 1..12: note = an artificially synthesised sequence
TSTSGRSASPRG

SEQ ID NO: 271 - protein, 12 aa, synthetic construct
  REGION 1..12: note = an artificially synthesised sequence
TSTSGRSATPRG

SEQ ID NO: 272 - protein, 12 aa, synthetic construct
  REGION 1..12: note = an artificially synthesised sequence
TSTSGRSANHRG

SEQ ID NO: 273 - protein, 12 aa, synthetic construct
  REGION 1..12: note = an artificially synthesised sequence
TSTSGRSANLRG

SEQ ID NO: 274 - protein, 12 aa, synthetic construct
  REGION 1..12: note = an artificially synthesised sequence
TSTSGRSANMRG

SEQ ID NO: 275 - protein, 12 aa, synthetic construct
  REGION 1..12: note = an artificially synthesised sequence
TSTSGRSANRRG

SEQ ID NO: 276 - protein, 12 aa, synthetic construct
  REGION 1..12: note = an artificially synthesised sequence
TSTSGRSANVRG

SEQ ID NO: 277 - protein, 12 aa, synthetic construct
  REGION 1..12: note = an artificially synthesised sequence
TSTSGRSANYRG

SEQ ID NO: 278 - protein, 12 aa, synthetic construct
  REGION 1..12: note = an artificially synthesised sequence
TSGSGRSAVPRG

SEQ ID NO: 279 - protein, 12 aa, synthetic construct
  REGION 1..12: note = an artificially synthesised sequence
TSGSGRSAYPRG

SEQ ID NO: 280 - protein, 12 aa, synthetic construct
  REGION 1..12: note = an artificially synthesised sequence
TSGSGRSANQRG

SEQ ID NO: 281 - protein, 12 aa, synthetic construct
  REGION 1..12: note = an artificially synthesised sequence
TSGSGRSANIRG

SEQ ID NO: 282 - protein, 12 aa, synthetic construct
  REGION 1..12: note = an artificially synthesised sequence
TSGSGRSANFRG

SEQ ID NO: 283 - protein, 12 aa, synthetic construct
  REGION 1..12: note = an artificially synthesised sequence
TSGSGRSANSRG

SEQ ID NO: 284 - protein, 12 aa, synthetic construct
  REGION 1..12: note = an artificially synthesised sequence
TSQSGRSAVPRG

SEQ ID NO: 285 - protein, 12 aa, synthetic construct
  REGION 1..12: note = an artificially synthesised sequence
TSQSGRSAYPRG

SEQ ID NO: 286 - protein, 12 aa, synthetic construct
  REGION 1..12: note = an artificially synthesised sequence
TSQSGRSANARG

SEQ ID NO: 287 - protein, 12 aa, synthetic construct
  REGION 1..12: note = an artificially synthesised sequence
TSQSGRSANIRG

SEQ ID NO: 288 - protein, 12 aa, synthetic construct
  REGION 1..12: note = an artificially synthesised sequence
TSQSGRSANFRG

SEQ ID NO: 289 - protein, 12 aa, synthetic construct
  REGION 1..12: note = an artificially synthesised sequence
TSQSGRSANSRG

SEQ ID NO: 290 - protein, 12 aa, synthetic construct
  REGION 1..12: note = an artificially synthesised sequence
TSPSGRSAVPRG

SEQ ID NO: 291 - protein, 12 aa, synthetic construct
  REGION 1..12: note = an artificially synthesised sequence
TSPSGRSANQRG

SEQ ID NO: 292 - protein, 12 aa, synthetic construct
  REGION 1..12: note = an artificially synthesised sequence
TSPSGRSANARG

SEQ ID NO: 293 - protein, 12 aa, synthetic construct
  REGION 1..12: note = an artificially synthesised sequence
TSPSGRSANIRG

SEQ ID NO: 294 - protein, 12 aa, synthetic construct
  REGION 1..12: note = an artificially synthesised sequence
TSPSGRSANFRG

SEQ ID NO: 295 - protein, 12 aa, synthetic construct
  REGION 1..12: note = an artificially synthesised sequence
TSPSGRSANSRG

SEQ ID NO: 296 - protein, 12 aa, synthetic construct
  REGION 1..12: note = an artificially synthesised sequence
TSASGRSAVPRG

SEQ ID NO: 297 - protein, 12 aa, synthetic construct
  REGION 1..12: note = an artificially synthesised sequence
TSASGRSAYPRG

SEQ ID NO: 298 - protein, 12 aa, synthetic construct
  REGION 1..12: note = an artificially synthesised sequence
TSASGRSANQRG

SEQ ID NO: 299 - protein, 12 aa, synthetic construct
  REGION 1..12: note = an artificially synthesised sequence
TSASGRSANARG

SEQ ID NO: 300 - protein, 12 aa, synthetic construct
  REGION 1..12: note = an artificially synthesised sequence
TSASGRSANIRG

SEQ ID NO: 301 - protein, 12 aa, synthetic construct
  REGION 1..12: note = an artificially synthesised sequence
TSASGRSANFRG

SEQ ID NO: 302 - protein, 12 aa, synthetic construct
  REGION 1..12: note = an artificially synthesised sequence
TSASGRSANSRG

SEQ ID NO: 303 - protein, 12 aa, synthetic construct
  REGION 1..12: note = an artificially synthesised sequence
TSYSGRSENPRG

SEQ ID NO: 304 - protein, 12 aa, synthetic construct
  REGION 1..12: note = an artificially synthesised sequence
TSGSGRSENPRG

SEQ ID NO: 305 - protein, 12 aa, synthetic construct
  REGION 1..12: note = an artificially synthesised sequence
TSQSGRSENPRG

SEQ ID NO: 306 - protein, 12 aa, synthetic construct
  REGION 1..12: note = an artificially synthesised sequence
TSPSGRSENPRG

SEQ ID NO: 307 - protein, 12 aa, synthetic construct
  REGION 1..12: note = an artificially synthesised sequence
TSASGRSENPRG

SEQ ID NO: 308 - protein, 12 aa, synthetic construct
  REGION 1..12: note = an artificially synthesised sequence
TSHSGRSENPRG

SEQ ID NO: 309 - protein, 12 aa, synthetic construct
  REGION 1..12: note = an artificially synthesised sequence
TSTSGRSENQRG

SEQ ID NO: 310 - protein, 12 aa, synthetic construct
  REGION 1..12: note = an artificially synthesised sequence
TSTSGRSENARG

SEQ ID NO: 311 - protein, 12 aa, synthetic construct
  REGION 1..12: note = an artificially synthesised sequence
TSTSGRSENIRG

SEQ ID NO: 312 - protein, 12 aa, synthetic construct
  REGION 1..12: note = an artificially synthesised sequence
TSTSGRSENFRG

SEQ ID NO: 313 - protein, 12 aa, synthetic construct
  REGION 1..12: note = an artificially synthesised sequence
TSTSGRSENSRG

SEQ ID NO: 314 - protein, 12 aa, synthetic construct
  REGION 1..12: note = an artificially synthesised sequence
TSYSGRSAEPRG

SEQ ID NO: 315 - protein, 12 aa, synthetic construct
  REGION 1..12: note = an artificially synthesised sequence
TSGSGRSAEPRG

SEQ ID NO: 316 - protein, 12 aa, synthetic construct
  REGION 1..12: note = an artificially synthesised sequence
TSQSGRSAEPRG

SEQ ID NO: 317 - protein, 12 aa, synthetic construct
  REGION 1..12: note = an artificially synthesised sequence
TSPSGRSAEPRG

SEQ ID NO: 318 - protein, 12 aa, synthetic construct
  REGION 1..12: note = an artificially synthesised sequence
TSASGRSAEPRG

SEQ ID NO: 319 - protein, 12 aa, synthetic construct
  REGION 1..12: note = an artificially synthesised sequence
TSHSGRSAEPRG

SEQ ID NO: 320 - protein, 12 aa, synthetic construct
  REGION 1..12: note = an artificially synthesised sequence
TSTSGRSAEQRG

SEQ ID NO: 321 - protein, 12 aa, synthetic construct
  REGION 1..12: note = an artificially synthesised sequence
TSTSGRSAEARG

SEQ ID NO: 322 - protein, 12 aa, synthetic construct
  REGION 1..12: note = an artificially synthesised sequence
TSTSGRSAEIRG

SEQ ID NO: 323 - protein, 12 aa, synthetic construct
  REGION 1..12: note = an artificially synthesised sequence
TSTSGRSAEFRG

SEQ ID NO: 324 - protein, 12 aa, synthetic construct
  REGION 1..12: note = an artificially synthesised sequence
TSTSGRSAESRG

SEQ ID NO: 325 - protein, 12 aa, synthetic construct
  REGION 1..12: note = an artificially synthesised sequence
TSGTGRSANPRG

SEQ ID NO: 326 - protein, 12 aa, synthetic construct
  REGION 1..12: note = an artificially synthesised sequence
TSGKGRSANPRG

SEQ ID NO: 327 - protein, 12 aa, synthetic construct
  REGION 1..12: note = an artificially synthesised sequence
TSGSGRSAIPRG

SEQ ID NO: 328 - protein, 12 aa, synthetic construct
  REGION 1..12: note = an artificially synthesised sequence
TSGSGRSASPRG

SEQ ID NO: 329 - protein, 12 aa, synthetic construct
  REGION 1..12: note = an artificially synthesised sequence
TSGSGRSAHPRG

SEQ ID NO: 330 - protein, 12 aa, synthetic construct
  REGION 1..12: note = an artificially synthesised sequence
TSGSGRSANYRG

SEQ ID NO: 331 - protein, 12 aa, synthetic construct
  REGION 1..12: note = an artificially synthesised sequence
TSGSGRSANVRG

SEQ ID NO: 332 - protein, 12 aa, synthetic construct
  REGION 1..12: note = an artificially synthesised sequence
TSGSGRSANHRG

SEQ ID NO: 333 - protein, 12 aa, synthetic construct
  REGION 1..12: note = an artificially synthesised sequence
TSQTGRSANPRG

SEQ ID NO: 334 - protein, 12 aa, synthetic construct
  REGION 1..12: note = an artificially synthesised sequence
TSQKGRSANPRG

SEQ ID NO: 335 - protein, 12 aa, synthetic construct
  REGION 1..12: note = an artificially synthesised sequence
TSQSGRSAIPRG

SEQ ID NO: 336 - protein, 12 aa, synthetic construct
  REGION 1..12: note = an artificially synthesised sequence
TSQSGRSASPRG

SEQ ID NO: 337 - protein, 12 aa, synthetic construct
  REGION 1..12: note = an artificially synthesised sequence
TSQSGRSAHPRG

SEQ ID NO: 338 - protein, 12 aa, synthetic construct
  REGION 1..12: note = an artificially synthesised sequence
TSQSGRSANYRG

SEQ ID NO: 339 - protein, 12 aa, synthetic construct
  REGION 1..12: note = an artificially synthesised sequence
TSQSGRSANVRG

SEQ ID NO: 340 - protein, 12 aa, synthetic construct
  REGION 1..12: note = an artificially synthesised sequence
TSQSGRSANHRG

SEQ ID NO: 341 - protein, 12 aa, synthetic construct
  REGION 1..12: note = an artificially synthesised sequence
TSPTGRSANPRG

SEQ ID NO: 342 - protein, 12 aa, synthetic construct
  REGION 1..12: note = an artificially synthesised sequence
TSPKGRSANPRG

SEQ ID NO: 343 - protein, 12 aa, synthetic construct
  REGION 1..12: note = an artificially synthesised sequence
TSPSGRSAIPRG

SEQ ID NO: 344 - protein, 12 aa, synthetic construct
  REGION 1..12: note = an artificially synthesised sequence
TSPSGRSATPRG

SEQ ID NO: 345 - protein, 12 aa, synthetic construct
  REGION 1..12: note = an artificially synthesised sequence
TSPSGRSASPRG

SEQ ID NO: 346 - protein, 12 aa, synthetic construct
  REGION 1..12: note = an artificially synthesised sequence
TSPSGRSAHPRG

SEQ ID NO: 347 - protein, 12 aa, synthetic construct
  REGION 1..12: note = an artificially synthesised sequence
TSPSGRSANYRG

SEQ ID NO: 348 - protein, 12 aa, synthetic construct
  REGION 1..12: note = an artificially synthesised sequence
TSPSGRSANVRG

SEQ ID NO: 349 - protein, 12 aa, synthetic construct
  REGION 1..12: note = an artificially synthesised sequence
TSPSGRSANHRG

SEQ ID NO: 350 - protein, 12 aa, synthetic construct
  REGION 1..12: note = an artificially synthesised sequence
TSATGRSANPRG

SEQ ID NO: 351 - protein, 12 aa, synthetic construct
  REGION 1..12: note = an artificially synthesised sequence
TSAKGRSANPRG

SEQ ID NO: 352 - protein, 12 aa, synthetic construct
  REGION 1..12: note = an artificially synthesised sequence
TSASGRSAIPRG

SEQ ID NO: 353 - protein, 12 aa, synthetic construct
  REGION 1..12: note = an artificially synthesised sequence
TSASGRSASPRG

SEQ ID NO: 354 - protein, 12 aa, synthetic construct
  REGION 1..12: note = an artificially synthesised sequence
TSASGRSAHPRG

SEQ ID NO: 355 - protein, 12 aa, synthetic construct
  REGION 1..12: note = an artificially synthesised sequence
TSASGRSANYRG

SEQ ID NO: 356 - protein, 12 aa, synthetic construct
  REGION 1..12: note = an artificially synthesised sequence
TSASGRSANVRG

SEQ ID NO: 357 - protein, 12 aa, synthetic construct
  REGION 1..12: note = an artificially synthesised sequence
TSASGRSANHRG

SEQ ID NO: 358 - protein, 12 aa, synthetic construct
  REGION 1..12: note = an artificially synthesised sequence
TSYTGRSANPRG

SEQ ID NO: 359 - protein, 12 aa, synthetic construct
  REGION 1..12: note = an artificially synthesised sequence
TSYKGRSANPRG

SEQ ID NO: 360 - protein, 12 aa, synthetic construct
  REGION 1..12: note = an artificially synthesised sequence
TSYSGRSAIPRG

SEQ ID NO: 361 - protein, 12 aa, synthetic construct
  REGION 1..12: note = an artificially synthesised sequence
TSYSGRSATPRG

SEQ ID NO: 362 - protein, 12 aa, synthetic construct
  REGION 1..12: note = an artificially synthesised sequence
TSYSGRSASPRG

SEQ ID NO: 363 - protein, 12 aa, synthetic construct
  REGION 1..12: note = an artificially synthesised sequence
TSYSGRSAHPRG

SEQ ID NO: 364 - protein, 12 aa, synthetic construct
  REGION 1..12: note = an artificially synthesised sequence
TSYSGRSANARG

SEQ ID NO: 365 - protein, 12 aa, synthetic construct
  REGION 1..12: note = an artificially synthesised sequence
TSYSGRSANYRG

SEQ ID NO: 366 - protein, 12 aa, synthetic construct
  REGION 1..12: note = an artificially synthesised sequence
TSYSGRSANVRG

SEQ ID NO: 367 - protein, 12 aa, synthetic construct
  REGION 1..12: note = an artificially synthesised sequence
TSYSGRSANHRG

SEQ ID NO: 368 - protein, 12 aa, synthetic construct
  REGION 1..12: note = an artificially synthesised sequence
TSSTGRSANPRG

SEQ ID NO: 369 - protein, 12 aa, synthetic construct
  REGION 1..12: note = an artificially synthesised sequence
TSSKGRSANPRG

SEQ ID NO: 370 - protein, 12 aa, synthetic construct
  REGION 1..12: note = an artificially synthesised sequence
TSSSGRSAVPRG

SEQ ID NO: 371 - protein, 12 aa, synthetic construct
  REGION 1..12: note = an artificially synthesised sequence
TSSSGRSAIPRG

SEQ ID NO: 372 - protein, 12 aa, synthetic construct
  REGION 1..12: note = an artificially synthesised sequence
TSSSGRSASPRG

SEQ ID NO: 373 - protein, 12 aa, synthetic construct
  REGION 1..12: note = an artificially synthesised sequence
TSSSGRSAHPRG

SEQ ID NO: 374 - protein, 12 aa, synthetic construct
  REGION 1..12: note = an artificially synthesised sequence
TSSSGRSANARG

SEQ ID NO: 375 - protein, 12 aa, synthetic construct
  REGION 1..12: note = an artificially synthesised sequence
TSSSGRSANFRG

SEQ ID NO: 376 - protein, 12 aa, synthetic construct
  REGION 1..12: note = an artificially synthesised sequence
TSSSGRSANYRG

SEQ ID NO: 377 - protein, 12 aa, synthetic construct
  REGION 1..12: note = an artificially synthesised sequence
TSSSGRSANVRG

SEQ ID NO: 378 - protein, 12 aa, synthetic construct
  REGION 1..12: note = an artificially synthesised sequence
TSSSGRSANHRG

SEQ ID NO: 379 - protein, 12 aa, synthetic construct
  REGION 1..12: note = an artificially synthesised sequence
TSITGRSANPRG

SEQ ID NO: 380 - protein, 12 aa, synthetic construct
  REGION 1..12: note = an artificially synthesised sequence
TSIKGRSANPRG

SEQ ID NO: 381 - protein, 12 aa, synthetic construct
  REGION 1..12: note = an artificially synthesised sequence
TSISGRSAVPRG

SEQ ID NO: 382 - protein, 12 aa, synthetic construct
  REGION 1..12: note = an artificially synthesised sequence
TSISGRSAIPRG

SEQ ID NO: 383 - protein, 12 aa, synthetic construct
  REGION 1..12: note = an artificially synthesised sequence
TSISGRSATPRG

SEQ ID NO: 384 - protein, 12 aa, synthetic construct
  REGION 1..12: note = an artificially synthesised sequence
TSISGRSASPRG

SEQ ID NO: 385 - protein, 12 aa, synthetic construct
  REGION 1..12: note = an artificially synthesised sequence
TSISGRSAHPRG

SEQ ID NO: 386 - protein, 12 aa, synthetic construct
  REGION 1..12: note = an artificially synthesised sequence
TSISGRSANARG

SEQ ID NO: 387 - protein, 12 aa, synthetic construct
  REGION 1..12: note = an artificially synthesised sequence
TSISGRSANFRG

SEQ ID NO: 388 - protein, 12 aa, synthetic construct
  REGION 1..12: note = an artificially synthesised sequence
TSISGRSANYRG

SEQ ID NO: 389 - protein, 12 aa, synthetic construct
  REGION 1..12: note = an artificially synthesised sequence
TSISGRSANVRG

SEQ ID NO: 390 - protein, 12 aa, synthetic construct
  REGION 1..12: note = an artificially synthesised sequence
TSISGRSANHRG

SEQ ID NO: 391 - protein, 12 aa, synthetic construct
  REGION 1..12: note = an artificially synthesised sequence
TSTTGRSAVPRG

SEQ ID NO: 392 - protein, 12 aa, synthetic construct
  REGION 1..12: note = an artificially synthesised sequence
TSTTGRSAIPRG

SEQ ID NO: 393 - protein, 12 aa, synthetic construct
  REGION 1..12: note = an artificially synthesised sequence
TSTTGRSATPRG

SEQ ID NO: 394 - protein, 12 aa, synthetic construct
  REGION 1..12: note = an artificially synthesised sequence
TSTTGRSAHPRG

SEQ ID NO: 395 - protein, 12 aa, synthetic construct
  REGION 1..12: note = an artificially synthesised sequence
TSTTGRSANARG

SEQ ID NO: 396 - protein, 12 aa, synthetic construct
  REGION 1..12: note = an artificially synthesised sequence
TSTTGRSANFRG

SEQ ID NO: 397 - protein, 12 aa, synthetic construct
  REGION 1..12: note = an artificially synthesised sequence
TSTTGRSANYRG

SEQ ID NO: 398 - protein, 12 aa, synthetic construct
  REGION 1..12: note = an artificially synthesised sequence
TSTTGRSANVRG

SEQ ID NO: 399 - protein, 12 aa, synthetic construct
  REGION 1..12: note = an artificially synthesised sequence
TSTTGRSANHRG

SEQ ID NO: 400 - protein, 12 aa, synthetic construct
  REGION 1..12: note = an artificially synthesised sequence
TSTKGRSAVPRG

SEQ ID NO: 401 - protein, 12 aa, synthetic construct
  REGION 1..12: note = an artificially synthesised sequence
TSTKGRSAIPRG

SEQ ID NO: 402 - protein, 12 aa, synthetic construct
  REGION 1..12: note = an artificially synthesised sequence
TSTKGRSATPRG

SEQ ID NO: 403 - protein, 12 aa, synthetic construct
  REGION 1..12: note = an artificially synthesised sequence
TSTKGRSASPRG

SEQ ID NO: 404 - protein, 12 aa, synthetic construct
  REGION 1..12: note = an artificially synthesised sequence
TSTKGRSAHPRG

SEQ ID NO: 405 - protein, 12 aa, synthetic construct
  REGION 1..12: note = an artificially synthesised sequence
TSTKGRSANARG

SEQ ID NO: 406 - protein, 12 aa, synthetic construct
  REGION 1..12: note = an artificially synthesised sequence
TSTKGRSANFRG

SEQ ID NO: 407 - protein, 12 aa, synthetic construct
  REGION 1..12: note = an artificially synthesised sequence
TSTKGRSANYRG

SEQ ID NO: 408 - protein, 12 aa, synthetic construct
  REGION 1..12: note = an artificially synthesised sequence
TSTKGRSANVRG

SEQ ID NO: 409 - protein, 12 aa, synthetic construct
  REGION 1..12: note = an artificially synthesised sequence
TSTKGRSANHRG

SEQ ID NO: 410 - protein, 12 aa, synthetic construct
  REGION 1..12: note = an artificially synthesised sequence
TSTSGRSAVYRG

SEQ ID NO: 411 - protein, 12 aa, synthetic construct
  REGION 1..12: note = an artificially synthesised sequence
TSTSGRSAVVRG

SEQ ID NO: 412 - protein, 12 aa, synthetic construct
  REGION 1..12: note = an artificially synthesised sequence
TSTSGRSAVHRG

SEQ ID NO: 413 - protein, 12 aa, synthetic construct
  REGION 1..12: note = an artificially synthesised sequence
TSTSGRSAIYRG

SEQ ID NO: 414 - protein, 12 aa, synthetic construct
  REGION 1..12: note = an artificially synthesised sequence
TSTSGRSAIVRG

SEQ ID NO: 415 - protein, 12 aa, synthetic construct
  REGION 1..12: note = an artificially synthesised sequence
TSTSGRSAIHRG

SEQ ID NO: 416 - protein, 12 aa, synthetic construct
  REGION 1..12: note = an artificially synthesised sequence
TSTSGRSASYRG

SEQ ID NO: 417 - protein, 12 aa, synthetic construct
  REGION 1..12: note = an artificially synthesised sequence
TSTSGRSASVRG

SEQ ID NO: 418 - protein, 12 aa, synthetic construct
  REGION 1..12: note = an artificially synthesised sequence
TSTSGRSASHRG

SEQ ID NO: 419 - protein, 12 aa, synthetic construct
  REGION 1..12: note = an artificially synthesised sequence
TSTSGRSAHYRG

SEQ ID NO: 420 - protein, 12 aa, synthetic construct
  REGION 1..12: note = an artificially synthesised sequence
TSTSGRSAHVRG

SEQ ID NO: 421 - protein, 12 aa, synthetic construct
  REGION 1..12: note = an artificially synthesised sequence
TSTSGRSAHHRG

SEQ ID NO: 422 - protein, 12 aa, synthetic construct
  REGION 1..12: note = an artificially synthesised sequence
TSPSGRSEVPRG

SEQ ID NO: 423 - protein, 12 aa, synthetic construct
  REGION 1..12: note = an artificially synthesised sequence
TSPSGRSAEPRG

SEQ ID NO: 424 - protein, 12 aa, synthetic construct
  REGION 1..12: note = an artificially synthesised sequence
TSPSGRSAGPRG

SEQ ID NO: 425 - protein, 12 aa, synthetic construct
  REGION 1..12: note = an artificially synthesised sequence
TSASGRSENARG

SEQ ID NO: 426 - protein, 12 aa, synthetic construct
  REGION 1..12: note = an artificially synthesised sequence
TSASGRSAEARG

SEQ ID NO: 427 - protein, 12 aa, synthetic construct
  REGION 1..12: note = an artificially synthesised sequence
TSASGRSAGARG

SEQ ID NO: 428 - protein, 12 aa, synthetic construct
  REGION 1..12: note = an artificially synthesised sequence
TSGTGRSATPRG

SEQ ID NO: 429 - protein, 12 aa, synthetic construct
  REGION 1..12: note = an artificially synthesised sequence
TSGSGRSATYRG

SEQ ID NO: 430 - protein, 12 aa, synthetic construct
  REGION 1..12: note = an artificially synthesised sequence
TSGSGRSATVRG

SEQ ID NO: 431 - protein, 12 aa, synthetic construct
  REGION 1..12: note = an artificially synthesised sequence
TSGSGRSATHRG

SEQ ID NO: 432 - protein, 12 aa, synthetic construct
  REGION 1..12: note = an artificially synthesised sequence
TSGTGRSATYRG

SEQ ID NO: 433 - protein, 12 aa, synthetic construct
  REGION 1..12: note = an artificially synthesised sequence
TSGTGRSATVRG

SEQ ID NO: 434 - protein, 12 aa, synthetic construct
  REGION 1..12: note = an artificially synthesised sequence
TSGTGRSATHRG

SEQ ID NO: 435 - protein, 12 aa, synthetic construct
  REGION 1..12: note = an artificially synthesised sequence
TSGSGRSETPRG

SEQ ID NO: 436 - protein, 12 aa, synthetic construct
  REGION 1..12: note = an artificially synthesised sequence
TSGTGRSETPRG

SEQ ID NO: 437 - protein, 12 aa, synthetic construct
  REGION 1..12: note = an artificially synthesised sequence
TSGSGRSETYRG

SEQ ID NO: 438 - protein, 12 aa, synthetic construct
  REGION 1..12: note = an artificially synthesised sequence
TSGSGRSETVRG

SEQ ID NO: 439 - protein, 12 aa, synthetic construct
  REGION 1..12: note = an artificially synthesised sequence
TSGSGRSETHRG

SEQ ID NO: 440 - protein, 12 aa, synthetic construct
  REGION 1..12: note = an artificially synthesised sequence
TSYTGRSAVPRG

SEQ ID NO: 441 - protein, 12 aa, synthetic construct
  REGION 1..12: note = an artificially synthesised sequence
TSYSGRSAVYRG

SEQ ID NO: 442 - protein, 12 aa, synthetic construct
  REGION 1..12: note = an artificially synthesised sequence
TSYSGRSAVVRG

SEQ ID NO: 443 - protein, 12 aa, synthetic construct
  REGION 1..12: note = an artificially synthesised sequence
TSYSGRSAVHRG

SEQ ID NO: 444 - protein, 12 aa, synthetic construct
  REGION 1..12: note = an artificially synthesised sequence
TSYTGRSAVYRG

SEQ ID NO: 445 - protein, 12 aa, synthetic construct
  REGION 1..12: note = an artificially synthesised sequence
TSYTGRSAVVRG

SEQ ID NO: 446 - protein, 12 aa, synthetic construct
  REGION 1..12: note = an artificially synthesised sequence
TSYTGRSAVHRG

SEQ ID NO: 447 - protein, 12 aa, synthetic construct
  REGION 1..12: note = an artificially synthesised sequence
TSYSGRSEVPRG

SEQ ID NO: 448 - protein, 12 aa, synthetic construct
  REGION 1..12: note = an artificially synthesised sequence
TSYTGRSEVPRG

SEQ ID NO: 449 - protein, 12 aa, synthetic construct
  REGION 1..12: note = an artificially synthesised sequence
TSYSGRSEVYRG

SEQ ID NO: 450 - protein, 12 aa, synthetic construct
  REGION 1..12: note = an artificially synthesised sequence
TSYSGRSEVVRG

SEQ ID NO: 451 - protein, 12 aa, synthetic construct
  REGION 1..12: note = an artificially synthesised sequence
TSYSGRSEVHRG

SEQ ID NO: 452 - protein, 12 aa, synthetic construct
  REGION 1..12: note = an artificially synthesised sequence
TSYTGRSAVPGG

SEQ ID NO: 453 - protein, 12 aa, synthetic construct
  REGION 1..12: note = an artificially synthesised sequence
TSYSGRSAVYGG

SEQ ID NO: 454 - protein, 12 aa, synthetic construct
  REGION 1..12: note = an artificially synthesised sequence
TSYSGRSAVVGG

SEQ ID NO: 455 - protein, 12 aa, synthetic construct
  REGION 1..12: note = an artificially synthesised sequence
TSYSGRSAVHGG

SEQ ID NO: 456 - protein, 12 aa, synthetic construct
  REGION 1..12: note = an artificially synthesised sequence
TSYTGRSAVYGG

SEQ ID NO: 457 - protein, 12 aa, synthetic construct
  REGION 1..12: note = an artificially synthesised sequence
TSYTGRSAVVGG

SEQ ID NO: 458 - protein, 12 aa, synthetic construct
  REGION 1..12: note = an artificially synthesised sequence
TSYTGRSAVHGG

SEQ ID NO: 459 - protein, 8 aa, synthetic construct
  REGION 1..8: note = an artificially synthesised sequence
ASGRSANP

SEQ ID NO: 460 - protein, 8 aa, synthetic construct
  REGION 1..8: note = an artificially synthesised sequence
ESGRSANP

SEQ ID NO: 461 - protein, 8 aa, synthetic construct
  REGION 1..8: note = an artificially synthesised sequence
FSGRSANP

SEQ ID NO: 462 - protein, 8 aa, synthetic construct
  REGION 1..8: note = an artificially synthesised sequence
GSGRSANP

SEQ ID NO: 463 - protein, 8 aa, synthetic construct
  REGION 1..8: note = an artificially synthesised sequence
HSGRSANP

SEQ ID NO: 464 - protein, 8 aa, synthetic construct
  REGION 1..8: note = an artificially synthesised sequence
KSGRSANP

SEQ ID NO: 465 - protein, 8 aa, synthetic construct
  REGION 1..8: note = an artificially synthesised sequence
MSGRSANP

SEQ ID NO: 466 - protein, 8 aa, synthetic construct
  REGION 1..8: note = an artificially synthesised sequence
NSGRSANP

SEQ ID NO: 467 - protein, 8 aa, synthetic construct
  REGION 1..8: note = an artificially synthesised sequence
PSGRSANP

SEQ ID NO: 468 - protein, 8 aa, synthetic construct
  REGION 1..8: note = an artificially synthesised sequence
QSGRSANP

SEQ ID NO: 469 - protein, 8 aa, synthetic construct
  REGION 1..8: note = an artificially synthesised sequence
WSGRSANP

SEQ ID NO: 470 - protein, 8 aa, synthetic construct
  REGION 1..8: note = an artificially synthesised sequence
YSGRSANP

SEQ ID NO: 471 - protein, 8 aa, synthetic construct
  REGION 1..8: note = an artificially synthesised sequence
TAGRSANP

SEQ ID NO: 472 - protein, 8 aa, synthetic construct
  REGION 1..8: note = an artificially synthesised sequence
TDGRSANP

SEQ ID NO: 473 - protein, 8 aa, synthetic construct
  REGION 1..8: note = an artificially synthesised sequence
TEGRSANP

SEQ ID NO: 474 - protein, 8 aa, synthetic construct
  REGION 1..8: note = an artificially synthesised sequence
TFGRSANP

SEQ ID NO: 475 - protein, 8 aa, synthetic construct
  REGION 1..8: note = an artificially synthesised sequence
TLGRSANP

SEQ ID NO: 476 - protein, 8 aa, synthetic construct
  REGION 1..8: note = an artificially synthesised sequence
TMGRSANP

SEQ ID NO: 477 - protein, 8 aa, synthetic construct
  REGION 1..8: note = an artificially synthesised sequence
TPGRSANP

SEQ ID NO: 478 - protein, 8 aa, synthetic construct
  REGION 1..8: note = an artificially synthesised sequence
TQGRSANP

SEQ ID NO: 479 - protein, 8 aa, synthetic construct
  REGION 1..8: note = an artificially synthesised sequence
TVGRSANP

SEQ ID NO: 480 - protein, 8 aa, synthetic construct
  REGION 1..8: note = an artificially synthesised sequence
TWGRSANP

SEQ ID NO: 481 - protein, 8 aa, synthetic construct
  REGION 1..8: note = an artificially synthesised sequence
TSARSANP

SEQ ID NO: 482 - protein, 8 aa, synthetic construct
  REGION 1..8: note = an artificially synthesised sequence
TSERSANP

SEQ ID NO: 483 - protein, 8 aa, synthetic construct
  REGION 1..8: note = an artificially synthesised sequence
TSFRSANP

SEQ ID NO: 484 - protein, 8 aa, synthetic construct
  REGION 1..8: note = an artificially synthesised sequence
TSHRSANP

SEQ ID NO: 485 - protein, 8 aa, synthetic construct
  REGION 1..8: note = an artificially synthesised sequence
TSIRSANP

SEQ ID NO: 486 - protein, 8 aa, synthetic construct
  REGION 1..8: note = an artificially synthesised sequence
TSKRSANP

SEQ ID NO: 487 - protein, 8 aa, synthetic construct
  REGION 1..8: note = an artificially synthesised sequence
TSLRSANP

SEQ ID NO: 488 - protein, 8 aa, synthetic construct
  REGION 1..8: note = an artificially synthesised sequence
TSMRSANP

SEQ ID NO: 489 - protein, 8 aa, synthetic construct
  REGION 1..8: note = an artificially synthesised sequence
TSNRSANP

SEQ ID NO: 490 - protein, 8 aa, synthetic construct
  REGION 1..8: note = an artificially synthesised sequence
TSPRSANP

SEQ ID NO: 491 - protein, 8 aa, synthetic construct
  REGION 1..8: note = an artificially synthesised sequence
TSQRSANP

SEQ ID NO: 492 - protein, 8 aa, synthetic construct
  REGION 1..8: note = an artificially synthesised sequence
TSRRSANP

SEQ ID NO: 493 - protein, 8 aa, synthetic construct
  REGION 1..8: note = an artificially synthesised sequence
TSTRSANP

SEQ ID NO: 494 - protein, 8 aa, synthetic construct
  REGION 1..8: note = an artificially synthesised sequence
TSVRSANP

SEQ ID NO: 495 - protein, 8 aa, synthetic construct
  REGION 1..8: note = an artificially synthesised sequence
TSWRSANP

SEQ ID NO: 496 - protein, 8 aa, synthetic construct
  REGION 1..8: note = an artificially synthesised sequence
TSYRSANP

SEQ ID NO: 497 - protein, 8 aa, synthetic construct
  REGION 1..8: note = an artificially synthesised sequence
TSGRAANP

SEQ ID NO: 498 - protein, 8 aa, synthetic construct
  REGION 1..8: note = an artificially synthesised sequence
TSGRDANP

SEQ ID NO: 499 - protein, 8 aa, synthetic construct
  REGION 1..8: note = an artificially synthesised sequence
TSGREANP

SEQ ID NO: 500 - protein, 8 aa, synthetic construct
  REGION 1..8: note = an artificially synthesised sequence
TSGRGANP

SEQ ID NO: 501 - protein, 8 aa, synthetic construct
  REGION 1..8: note = an artificially synthesised sequence
TSGRHANP

SEQ ID NO: 502 - protein, 8 aa, synthetic construct
  REGION 1..8: note = an artificially synthesised sequence
TSGRIANP

SEQ ID NO: 503 - protein, 8 aa, synthetic construct
  REGION 1..8: note = an artificially synthesised sequence
TSGRKANP

SEQ ID NO: 504 - protein, 8 aa, synthetic construct
  REGION 1..8: note = an artificially synthesised sequence
TSGRLANP

SEQ ID NO: 505 - protein, 8 aa, synthetic construct
  REGION 1..8: note = an artificially synthesised sequence
TSGRMANP

SEQ ID NO: 506 - protein, 8 aa, synthetic construct
  REGION 1..8: note = an artificially synthesised sequence
TSGRNANP

SEQ ID NO: 507 - protein, 8 aa, synthetic construct
  REGION 1..8: note = an artificially synthesised sequence
TSGRPANP

SEQ ID NO: 508 - protein, 8 aa, synthetic construct
  REGION 1..8: note = an artificially synthesised sequence
TSGRQANP

SEQ ID NO: 509 - protein, 8 aa, synthetic construct
  REGION 1..8: note = an artificially synthesised sequence
TSGRRANP

SEQ ID NO: 510 - protein, 8 aa, synthetic construct
  REGION 1..8: note = an artificially synthesised sequence
TSGRTANP

SEQ ID NO: 511 - protein, 8 aa, synthetic construct
  REGION 1..8: note = an artificially synthesised sequence
TSGRVANP

SEQ ID NO: 512 - protein, 8 aa, synthetic construct
  REGION 1..8: note = an artificially synthesised sequence
TSGRWANP

SEQ ID NO: 513 - protein, 8 aa, synthetic construct
  REGION 1..8: note = an artificially synthesised sequence
TSGRYANP

SEQ ID NO: 514 - protein, 8 aa, synthetic construct
  REGION 1..8: note = an artificially synthesised sequence
TSGRSENP

SEQ ID NO: 515 - protein, 8 aa, synthetic construct
  REGION 1..8: note = an artificially synthesised sequence
TSGRSFNP

SEQ ID NO: 516 - protein, 8 aa, synthetic construct
  REGION 1..8: note = an artificially synthesised sequence
TSGRSKNP

SEQ ID NO: 517 - protein, 8 aa, synthetic construct
  REGION 1..8: note = an artificially synthesised sequence
TSGRSMNP

SEQ ID NO: 518 - protein, 8 aa, synthetic construct
  REGION 1..8: note = an artificially synthesised sequence
TSGRSNNP

SEQ ID NO: 519 - protein, 8 aa, synthetic construct
  REGION 1..8: note = an artificially synthesised sequence
TSGRSPNP

SEQ ID NO: 520 - protein, 8 aa, synthetic construct
  REGION 1..8: note = an artificially synthesised sequence
TSGRSQNP

SEQ ID NO: 521 - protein, 8 aa, synthetic construct
  REGION 1..8: note = an artificially synthesised sequence
TSGRSRNP

SEQ ID NO: 522 - protein, 8 aa, synthetic construct
  REGION 1..8: note = an artificially synthesised sequence
TSGRSSNP

SEQ ID NO: 523 - protein, 8 aa, synthetic construct
  REGION 1..8: note = an artificially synthesised sequence
TSGRSWNP

SEQ ID NO: 524 - protein, 8 aa, synthetic construct
  REGION 1..8: note = an artificially synthesised sequence
TSGRSYNP

SEQ ID NO: 525 - protein, 8 aa, synthetic construct
  REGION 1..8: note = an artificially synthesised sequence
TSGRSAAP

SEQ ID NO: 526 - protein, 8 aa, synthetic construct
  REGION 1..8: note = an artificially synthesised sequence
TSGRSADP

SEQ ID NO: 527 - protein, 8 aa, synthetic construct
  REGION 1..8: note = an artificially synthesised sequence
TSGRSAEP

SEQ ID NO: 528 - protein, 8 aa, synthetic construct
  REGION 1..8: note = an artificially synthesised sequence
TSGRSAFP

SEQ ID NO: 529 - protein, 8 aa, synthetic construct
  REGION 1..8: note = an artificially synthesised sequence
TSGRSAGP

SEQ ID NO: 530 - protein, 8 aa, synthetic construct
  REGION 1..8: note = an artificially synthesised sequence
TSGRSAKP

SEQ ID NO: 531 - protein, 8 aa, synthetic construct
  REGION 1..8: note = an artificially synthesised sequence
TSGRSALP

SEQ ID NO: 532 - protein, 8 aa, synthetic construct
  REGION 1..8: note = an artificially synthesised sequence
TSGRSAMP

SEQ ID NO: 533 - protein, 8 aa, synthetic construct
  REGION 1..8: note = an artificially synthesised sequence
TSGRSAPP

SEQ ID NO: 534 - protein, 8 aa, synthetic construct
  REGION 1..8: note = an artificially synthesised sequence
TSGRSAQP

SEQ ID NO: 535 - protein, 8 aa, synthetic construct
  REGION 1..8: note = an artificially synthesised sequence
TSGRSAVP

SEQ ID NO: 536 - protein, 8 aa, synthetic construct
  REGION 1..8: note = an artificially synthesised sequence
TSGRSAWP

SEQ ID NO: 537 - protein, 8 aa, synthetic construct
  REGION 1..8: note = an artificially synthesised sequence
TSGRSAYP

SEQ ID NO: 538 - protein, 8 aa, synthetic construct
  REGION 1..8: note = an artificially synthesised sequence
TSGRSANA

SEQ ID NO: 539 - protein, 8 aa, synthetic construct
  REGION 1..8: note = an artificially synthesised sequence
TSGRSAND

SEQ ID NO: 540 - protein, 8 aa, synthetic construct
  REGION 1..8: note = an artificially synthesised sequence
TSGRSANE

SEQ ID NO: 541 - protein, 8 aa, synthetic construct
  REGION 1..8: note = an artificially synthesised sequence
TSGRSANF

SEQ ID NO: 542 - protein, 8 aa, synthetic construct
  REGION 1..8: note = an artificially synthesised sequence
TSGRSANG

SEQ ID NO: 543 - protein, 8 aa, synthetic construct
  REGION 1..8: note = an artificially synthesised sequence
TSGRSANI

SEQ ID NO: 544 - protein, 8 aa, synthetic construct
  REGION 1..8: note = an artificially synthesised sequence
TSGRSANK

SEQ ID NO: 545 - protein, 8 aa, synthetic construct
  REGION 1..8: note = an artificially synthesised sequence
TSGRSANN

SEQ ID NO: 546 - protein, 8 aa, synthetic construct
  REGION 1..8: note = an artificially synthesised sequence
TSGRSANQ

SEQ ID NO: 547 - protein, 8 aa, synthetic construct
  REGION 1..8: note = an artificially synthesised sequence
TSGRSANS

SEQ ID NO: 548 - protein, 8 aa, synthetic construct
  REGION 1..8: note = an artificially synthesised sequence
TSGRSANT

SEQ ID NO: 549 - protein, 8 aa, synthetic construct
  REGION 1..8: note = an artificially synthesised sequence
TSGRSANW

SEQ ID NO: 550 - protein, 8 aa, synthetic construct
  REGION 1..8: note = an artificially synthesised sequence
DSGRSANP

SEQ ID NO: 551 - protein, 8 aa, synthetic construct
  REGION 1..8: note = an artificially synthesised sequence
ISGRSANP

SEQ ID NO: 552 - protein, 8 aa, synthetic construct
  REGION 1..8: note = an artificially synthesised sequence
SSGRSANP

SEQ ID NO: 553 - protein, 8 aa, synthetic construct
  REGION 1..8: note = an artificially synthesised sequence
THGRSANP

SEQ ID NO: 554 - protein, 8 aa, synthetic construct
  REGION 1..8: note = an artificially synthesised sequence
TKGRSANP

SEQ ID NO: 555 - protein, 8 aa, synthetic construct
  REGION 1..8: note = an artificially synthesised sequence
TTGRSANP

SEQ ID NO: 556 - protein, 8 aa, synthetic construct
  REGION 1..8: note = an artificially synthesised sequence
TYGRSANP

SEQ ID NO: 557 - protein, 8 aa, synthetic construct
  REGION 1..8: note = an artificially synthesised sequence
TSDRSANP

SEQ ID NO: 558 - protein, 8 aa, synthetic construct
  REGION 1..8: note = an artificially synthesised sequence
TSSRSANP

SEQ ID NO: 559 - protein, 8 aa, synthetic construct
  REGION 1..8: note = an artificially synthesised sequence
TSGRFANP

SEQ ID NO: 560 - protein, 8 aa, synthetic construct
  REGION 1..8: note = an artificially synthesised sequence
TSGRSDNP

SEQ ID NO: 561 - protein, 8 aa, synthetic construct
  REGION 1..8: note = an artificially synthesised sequence
TSGRSHNP

SEQ ID NO: 562 - protein, 8 aa, synthetic construct
  REGION 1..8: note = an artificially synthesised sequence
TSGRSINP

SEQ ID NO: 563 - protein, 8 aa, synthetic construct
  REGION 1..8: note = an artificially synthesised sequence
TSGRSLNP

SEQ ID NO: 564 - protein, 8 aa, synthetic construct
  REGION 1..8: note = an artificially synthesised sequence
TSGRSTNP

SEQ ID NO: 565 - protein, 8 aa, synthetic construct
  REGION 1..8: note = an artificially synthesised sequence
TSGRSVNP

SEQ ID NO: 566 - protein, 8 aa, synthetic construct
  REGION 1..8: note = an artificially synthesised sequence
TSGRSAHP

SEQ ID NO: 567 - protein, 8 aa, synthetic construct
  REGION 1..8: note = an artificially synthesised sequence
TSGRSAIP

SEQ ID NO: 568 - protein, 8 aa, synthetic construct
  REGION 1..8: note = an artificially synthesised sequence
TSGRSARP

SEQ ID NO: 569 - protein, 8 aa, synthetic construct
  REGION 1..8: note = an artificially synthesised sequence
TSGRSASP

SEQ ID NO: 570 - protein, 8 aa, synthetic construct
  REGION 1..8: note = an artificially synthesised sequence
TSGRSATP

SEQ ID NO: 571 - protein, 8 aa, synthetic construct
  REGION 1..8: note = an artificially synthesised sequence
TSGRSANH

SEQ ID NO: 572 - protein, 8 aa, synthetic construct
  REGION 1..8: note = an artificially synthesised sequence
TSGRSANL

SEQ ID NO: 573 - protein, 8 aa, synthetic construct
  REGION 1..8: note = an artificially synthesised sequence
TSGRSANM

SEQ ID NO: 574 - protein, 8 aa, synthetic construct
  REGION 1..8: note = an artificially synthesised sequence
TSGRSANR

SEQ ID NO: 575 - protein, 8 aa, synthetic construct
  REGION 1..8: note = an artificially synthesised sequence
TSGRSANV

SEQ ID NO: 576 - protein, 8 aa, synthetic construct
  REGION 1..8: note = an artificially synthesised sequence
TSGRSANY

SEQ ID NO: 577 - protein, 8 aa, synthetic construct
  REGION 1..8: note = an artificially synthesised sequence
GSGRSAVP

SEQ ID NO: 578 - protein, 8 aa, synthetic construct
  REGION 1..8: note = an artificially synthesised sequence
GSGRSAYP

SEQ ID NO: 579 - protein, 8 aa, synthetic construct
  REGION 1..8: note = an artificially synthesised sequence
GSGRSANQ

SEQ ID NO: 580 - protein, 8 aa, synthetic construct
  REGION 1..8: note = an artificially synthesised sequence
GSGRSANA

SEQ ID NO: 581 - protein, 8 aa, synthetic construct
  REGION 1..8: note = an artificially synthesised sequence
GSGRSANI

SEQ ID NO: 582 - protein, 8 aa, synthetic construct
  REGION 1..8: note = an artificially synthesised sequence
GSGRSANF

SEQ ID NO: 583 - protein, 8 aa, synthetic construct
  REGION 1..8: note = an artificially synthesised sequence
GSGRSANS

SEQ ID NO: 584 - protein, 8 aa, synthetic construct
  REGION 1..8: note = an artificially synthesised sequence
QSGRSAVP

SEQ ID NO: 585 - protein, 8 aa, synthetic construct
  REGION 1..8: note = an artificially synthesised sequence
QSGRSAYP

SEQ ID NO: 586 - protein, 8 aa, synthetic construct
  REGION 1..8: note = an artificially synthesised sequence
QSGRSANQ

SEQ ID NO: 587 - protein, 8 aa, synthetic construct
  REGION 1..8: note = an artificially synthesised sequence
QSGRSANA

SEQ ID NO: 588 - protein, 8 aa, synthetic construct
  REGION 1..8: note = an artificially synthesised sequence
QSGRSANI

SEQ ID NO: 589 - protein, 8 aa, synthetic construct
  REGION 1..8: note = an artificially synthesised sequence
QSGRSANF

SEQ ID NO: 590 - protein, 8 aa, synthetic construct
  REGION 1..8: note = an artificially synthesised sequence
QSGRSANS

SEQ ID NO: 591 - protein, 8 aa, synthetic construct
  REGION 1..8: note = an artificially synthesised sequence
PSGRSAVP

SEQ ID NO: 592 - protein, 8 aa, synthetic construct
  REGION 1..8: note = an artificially synthesised sequence
PSGRSAYP

SEQ ID NO: 593 - protein, 8 aa, synthetic construct
  REGION 1..8: note = an artificially synthesised sequence
PSGRSANQ

SEQ ID NO: 594 - protein, 8 aa, synthetic construct
  REGION 1..8: note = an artificially synthesised sequence
PSGRSANA

SEQ ID NO: 595 - protein, 8 aa, synthetic construct
  REGION 1..8: note = an artificially synthesised sequence
PSGRSANI

SEQ ID NO: 596 - protein, 8 aa, synthetic construct
  REGION 1..8: note = an artificially synthesised sequence
PSGRSANF

SEQ ID NO: 597 - protein, 8 aa, synthetic construct
  REGION 1..8: note = an artificially synthesised sequence
PSGRSANS

SEQ ID NO: 598 - protein, 8 aa, synthetic construct
  REGION 1..8: note = an artificially synthesised sequence
ASGRSAVP

SEQ ID NO: 599 - protein, 8 aa, synthetic construct
  REGION 1..8: note = an artificially synthesised sequence
ASGRSAYP

SEQ ID NO: 600 - protein, 8 aa, synthetic construct
  REGION 1..8: note = an artificially synthesised sequence
ASGRSANQ

SEQ ID NO: 601 - protein, 8 aa, synthetic construct
  REGION 1..8: note = an artificially synthesised sequence
ASGRSANA

SEQ ID NO: 602 - protein, 8 aa, synthetic construct
  REGION 1..8: note = an artificially synthesised sequence
ASGRSANI

SEQ ID NO: 603 - protein, 8 aa, synthetic construct
  REGION 1..8: note = an artificially synthesised sequence
ASGRSANF

SEQ ID NO: 604 - protein, 8 aa, synthetic construct
  REGION 1..8: note = an artificially synthesised sequence
ASGRSANS

SEQ ID NO: 605 - protein, 8 aa, synthetic construct
  REGION 1..8: note = an artificially synthesised sequence
YSGRSENP

SEQ ID NO: 606 - protein, 8 aa, synthetic construct
  REGION 1..8: note = an artificially synthesised sequence
GSGRSENP

SEQ ID NO: 607 - protein, 8 aa, synthetic construct
  REGION 1..8: note = an artificially synthesised sequence
QSGRSENP

SEQ ID NO: 608 - protein, 8 aa, synthetic construct
  REGION 1..8: note = an artificially synthesised sequence
PSGRSENP

SEQ ID NO: 609 - protein, 8 aa, synthetic construct
  REGION 1..8: note = an artificially synthesised sequence
ASGRSENP

SEQ ID NO: 610 - protein, 8 aa, synthetic construct
  REGION 1..8: note = an artificially synthesised sequence
HSGRSENP

SEQ ID NO: 611 - protein, 8 aa, synthetic construct
  REGION 1..8: note = an artificially synthesised sequence
TSGRSENQ

SEQ ID NO: 612 - protein, 8 aa, synthetic construct
  REGION 1..8: note = an artificially synthesised sequence
TSGRSENA

SEQ ID NO: 613 - protein, 8 aa, synthetic construct
  REGION 1..8: note = an artificially synthesised sequence
TSGRSENI

SEQ ID NO: 614 - protein, 8 aa, synthetic construct
  REGION 1..8: note = an artificially synthesised sequence
TSGRSENF

SEQ ID NO: 615 - protein, 8 aa, synthetic construct
  REGION 1..8: note = an artificially synthesised sequence
TSGRSENS

SEQ ID NO: 616 - protein, 8 aa, synthetic construct
  REGION 1..8: note = an artificially synthesised sequence
YSGRSAEP

SEQ ID NO: 617 - protein, 8 aa, synthetic construct
  REGION 1..8: note = an artificially synthesised sequence
GSGRSAEP

SEQ ID NO: 618 - protein, 8 aa, synthetic construct
  REGION 1..8: note = an artificially synthesised sequence
QSGRSAEP

SEQ ID NO: 619 - protein, 8 aa, synthetic construct
  REGION 1..8: note = an artificially synthesised sequence
PSGRSAEP

SEQ ID NO: 620 - protein, 8 aa, synthetic construct
  REGION 1..8: note = an artificially synthesised sequence
ASGRSAEP

SEQ ID NO: 621 - protein, 8 aa, synthetic construct
  REGION 1..8: note = an artificially synthesised sequence
HSGRSAEP

SEQ ID NO: 622 - protein, 8 aa, synthetic construct
  REGION 1..8: note = an artificially synthesised sequence
TSGRSAEQ

SEQ ID NO: 623 - protein, 8 aa, synthetic construct
  REGION 1..8: note = an artificially synthesised sequence
TSGRSAEA

SEQ ID NO: 624 - protein, 8 aa, synthetic construct
  REGION 1..8: note = an artificially synthesised sequence
TSGRSAEI

SEQ ID NO: 625 - protein, 8 aa, synthetic construct
  REGION 1..8: note = an artificially synthesised sequence
TSGRSAEF

SEQ ID NO: 626 - protein, 8 aa, synthetic construct
  REGION 1..8: note = an artificially synthesised sequence
TSGRSAES

SEQ ID NO: 627 - protein, 8 aa, synthetic construct
  REGION 1..8: note = an artificially synthesised sequence
GTGRSANP

SEQ ID NO: 628 - protein, 8 aa, synthetic construct
  REGION 1..8: note = an artificially synthesised sequence
GKGRSANP

SEQ ID NO: 629 - protein, 8 aa, synthetic construct
  REGION 1..8: note = an artificially synthesised sequence
GSGRSAIP

SEQ ID NO: 630 - protein, 8 aa, synthetic construct
  REGION 1..8: note = an artificially synthesised sequence
GSGRSATP

SEQ ID NO: 631 - protein, 8 aa, synthetic construct
  REGION 1..8: note = an artificially synthesised sequence
GSGRSASP

SEQ ID NO: 632 - protein, 8 aa, synthetic construct
  REGION 1..8: note = an artificially synthesised sequence
GSGRSAHP

SEQ ID NO: 633 - protein, 8 aa, synthetic construct
  REGION 1..8: note = an artificially synthesised sequence
GSGRSANY

SEQ ID NO: 634 - protein, 8 aa, synthetic construct
  REGION 1..8: note = an artificially synthesised sequence
GSGRSANV

SEQ ID NO: 635 - protein, 8 aa, synthetic construct
  REGION 1..8: note = an artificially synthesised sequence
GSGRSANH

SEQ ID NO: 636 - protein, 8 aa, synthetic construct
  REGION 1..8: note = an artificially synthesised sequence
QTGRSANP

SEQ ID NO: 637 - protein, 8 aa, synthetic construct
  REGION 1..8: note = an artificially synthesised sequence
QKGRSANP

SEQ ID NO: 638 - protein, 8 aa, synthetic construct
  REGION 1..8: note = an artificially synthesised sequence
QSGRSAIP

SEQ ID NO: 639 - protein, 8 aa, synthetic construct
  REGION 1..8: note = an artificially synthesised sequence
QSGRSATP

SEQ ID NO: 640 - protein, 8 aa, synthetic construct
  REGION 1..8: note = an artificially synthesised sequence
QSGRSASP

SEQ ID NO: 641 - protein, 8 aa, synthetic construct
  REGION 1..8: note = an artificially synthesised sequence
QSGRSAHP

SEQ ID NO: 642 - protein, 8 aa, synthetic construct
  REGION 1..8: note = an artificially synthesised sequence
QSGRSANY

SEQ ID NO: 643 - protein, 8 aa, synthetic construct
  REGION 1..8: note = an artificially synthesised sequence
QSGRSANV

SEQ ID NO: 644 - protein, 8 aa, synthetic construct
  REGION 1..8: note = an artificially synthesised sequence
QSGRSANH

SEQ ID NO: 645 - protein, 8 aa, synthetic construct
  REGION 1..8: note = an artificially synthesised sequence
PTGRSANP

SEQ ID NO: 646 - protein, 8 aa, synthetic construct
  REGION 1..8: note = an artificially synthesised sequence
PKGRSANP

SEQ ID NO: 647 - protein, 8 aa, synthetic construct
  REGION 1..8: note = an artificially synthesised sequence
PSGRSAIP

SEQ ID NO: 648 - protein, 8 aa, synthetic construct
  REGION 1..8: note = an artificially synthesised sequence
PSGRSATP

SEQ ID NO: 649 - protein, 8 aa, synthetic construct
  REGION 1..8: note = an artificially synthesised sequence
PSGRSASP

SEQ ID NO: 650 - protein, 8 aa, synthetic construct
  REGION 1..8: note = an artificially synthesised sequence
PSGRSAHP

SEQ ID NO: 651 - protein, 8 aa, synthetic construct
  REGION 1..8: note = an artificially synthesised sequence
PSGRSANY

SEQ ID NO: 652 - protein, 8 aa, synthetic construct
  REGION 1..8: note = an artificially synthesised sequence
PSGRSANV

SEQ ID NO: 653 - protein, 8 aa, synthetic construct
  REGION 1..8: note = an artificially synthesised sequence
PSGRSANH

SEQ ID NO: 654 - protein, 8 aa, synthetic construct
  REGION 1..8: note = an artificially synthesised sequence
ATGRSANP

SEQ ID NO: 655 - protein, 8 aa, synthetic construct
  REGION 1..8: note = an artificially synthesised sequence
AKGRSANP

SEQ ID NO: 656 - protein, 8 aa, synthetic construct
  REGION 1..8: note = an artificially synthesised sequence
ASGRSAIP

SEQ ID NO: 657 - protein, 8 aa, synthetic construct
  REGION 1..8: note = an artificially synthesised sequence
ASGRSATP

SEQ ID NO: 658 - protein, 8 aa, synthetic construct
  REGION 1..8: note = an artificially synthesised sequence
ASGRSASP

SEQ ID NO: 659 - protein, 8 aa, synthetic construct
  REGION 1..8: note = an artificially synthesised sequence
ASGRSAHP

SEQ ID NO: 660 - protein, 8 aa, synthetic construct
  REGION 1..8: note = an artificially synthesised sequence
ASGRSANY

SEQ ID NO: 661 - protein, 8 aa, synthetic construct
  REGION 1..8: note = an artificially synthesised sequence
ASGRSANV

SEQ ID NO: 662 - protein, 8 aa, synthetic construct
  REGION 1..8: note = an artificially synthesised sequence
ASGRSANH

SEQ ID NO: 663 - protein, 8 aa, synthetic construct
  REGION 1..8: note = an artificially synthesised sequence
YTGRSANP

SEQ ID NO: 664 - protein, 8 aa, synthetic construct
  REGION 1..8: note = an artificially synthesised sequence
YKGRSANP

SEQ ID NO: 665 - protein, 8 aa, synthetic construct
  REGION 1..8: note = an artificially synthesised sequence
YSGRSAVP

SEQ ID NO: 666 - protein, 8 aa, synthetic construct
  REGION 1..8: note = an artificially synthesised sequence
YSGRSAIP

SEQ ID NO: 667 - protein, 8 aa, synthetic construct
  REGION 1..8: note = an artificially synthesised sequence
YSGRSATP

SEQ ID NO: 668 - protein, 8 aa, synthetic construct
  REGION 1..8: note = an artificially synthesised sequence
YSGRSASP

SEQ ID NO: 669 - protein, 8 aa, synthetic construct
  REGION 1..8: note = an artificially synthesised sequence
YSGRSAHP

SEQ ID NO: 670 - protein, 8 aa, synthetic construct
  REGION 1..8: note = an artificially synthesised sequence
YSGRSANA

SEQ ID NO: 671 - protein, 8 aa, synthetic construct
  REGION 1..8: note = an artificially synthesised sequence
YSGRSANF

SEQ ID NO: 672 - protein, 8 aa, synthetic construct
  REGION 1..8: note = an artificially synthesised sequence
YSGRSANY

SEQ ID NO: 673 - protein, 8 aa, synthetic construct
  REGION 1..8: note = an artificially synthesised sequence
YSGRSANV

SEQ ID NO: 674 - protein, 8 aa, synthetic construct
  REGION 1..8: note = an artificially synthesised sequence
YSGRSANH

SEQ ID NO: 675 - protein, 8 aa, synthetic construct
  REGION 1..8: note = an artificially synthesised sequence
STGRSANP

SEQ ID NO: 676 - protein, 8 aa, synthetic construct
  REGION 1..8: note = an artificially synthesised sequence
SKGRSANP

SEQ ID NO: 677 - protein, 8 aa, synthetic construct
  REGION 1..8: note = an artificially synthesised sequence
SSGRSAVP

SEQ ID NO: 678 - protein, 8 aa, synthetic construct
  REGION 1..8: note = an artificially synthesised sequence
SSGRSAIP

SEQ ID NO: 679 - protein, 8 aa, synthetic construct
  REGION 1..8: note = an artificially synthesised sequence
SSGRSATP

SEQ ID NO: 680 - protein, 8 aa, synthetic construct
  REGION 1..8: note = an artificially synthesised sequence
SSGRSASP

SEQ ID NO: 681 - protein, 8 aa, synthetic construct
  REGION 1..8: note = an artificially synthesised sequence
SSGRSAHP

SEQ ID NO: 682 - protein, 8 aa, synthetic construct
  REGION 1..8: note = an artificially synthesised sequence
SSGRSANA

SEQ ID NO: 683 - protein, 8 aa, synthetic construct
  REGION 1..8: note = an artificially synthesised sequence
SSGRSANF

SEQ ID NO: 684 - protein, 8 aa, synthetic construct
  REGION 1..8: note = an artificially synthesised sequence
SSGRSANY

SEQ ID NO: 685 - protein, 8 aa, synthetic construct
  REGION 1..8: note = an artificially synthesised sequence
SSGRSANV

SEQ ID NO: 686 - protein, 8 aa, synthetic construct
  REGION 1..8: note = an artificially synthesised sequence
SSGRSANH

SEQ ID NO: 687 - protein, 8 aa, synthetic construct
  REGION 1..8: note = an artificially synthesised sequence
ITGRSANP

SEQ ID NO: 688 - protein, 8 aa, synthetic construct
  REGION 1..8: note = an artificially synthesised sequence
IKGRSANP

SEQ ID NO: 689 - protein, 8 aa, synthetic construct
  REGION 1..8: note = an artificially synthesised sequence
ISGRSAVP

SEQ ID NO: 690 - protein, 8 aa, synthetic construct
  REGION 1..8: note = an artificially synthesised sequence
ISGRSAIP

SEQ ID NO: 691 - protein, 8 aa, synthetic construct
  REGION 1..8: note = an artificially synthesised sequence
ISGRSATP

SEQ ID NO: 692 - protein, 8 aa, synthetic construct
  REGION 1..8: note = an artificially synthesised sequence
ISGRSASP

SEQ ID NO: 693 - protein, 8 aa, synthetic construct
  REGION 1..8: note = an artificially synthesised sequence
ISGRSAHP

SEQ ID NO: 694 - protein, 8 aa, synthetic construct
  REGION 1..8: note = an artificially synthesised sequence
ISGRSANA

SEQ ID NO: 695 - protein, 8 aa, synthetic construct
  REGION 1..8: note = an artificially synthesised sequence
ISGRSANF

SEQ ID NO: 696 - protein, 8 aa, synthetic construct
  REGION 1..8: note = an artificially synthesised sequence
ISGRSANY

SEQ ID NO: 697 - protein, 8 aa, synthetic construct
  REGION 1..8: note = an artificially synthesised sequence
ISGRSANV

SEQ ID NO: 698 - protein, 8 aa, synthetic construct
  REGION 1..8: note = an artificially synthesised sequence
ISGRSANH

SEQ ID NO: 699 - protein, 8 aa, synthetic construct
  REGION 1..8: note = an artificially synthesised sequence
TTGRSAVP

SEQ ID NO: 700 - protein, 8 aa, synthetic construct
  REGION 1..8: note = an artificially synthesised sequence
TTGRSAIP

SEQ ID NO: 701 - protein, 8 aa, synthetic construct
  REGION 1..8: note = an artificially synthesised sequence
TTGRSATP

SEQ ID NO: 702 - protein, 8 aa, synthetic construct
  REGION 1..8: note = an artificially synthesised sequence
TTGRSASP

SEQ ID NO: 703 - protein, 8 aa, synthetic construct
  REGION 1..8: note = an artificially synthesised sequence
TTGRSAHP

SEQ ID NO: 704 - protein, 8 aa, synthetic construct
  REGION 1..8: note = an artificially synthesised sequence
TTGRSANA

SEQ ID NO: 705 - protein, 8 aa, synthetic construct
  REGION 1..8: note = an artificially synthesised sequence
TTGRSANF

SEQ ID NO: 706 - protein, 8 aa, synthetic construct
  REGION 1..8: note = an artificially synthesised sequence
TTGRSANY

SEQ ID NO: 707 - protein, 8 aa, synthetic construct
  REGION 1..8: note = an artificially synthesised sequence
TTGRSANV

SEQ ID NO: 708 - protein, 8 aa, synthetic construct
  REGION 1..8: note = an artificially synthesised sequence
TTGRSANH

SEQ ID NO: 709 - protein, 8 aa, synthetic construct
  REGION 1..8: note = an artificially synthesised sequence
TKGRSAVP

SEQ ID NO: 710 - protein, 8 aa, synthetic construct
  REGION 1..8: note = an artificially synthesised sequence
TKGRSAIP

SEQ ID NO: 711 - protein, 8 aa, synthetic construct
  REGION 1..8: note = an artificially synthesised sequence
TKGRSATP

SEQ ID NO: 712 - protein, 8 aa, synthetic construct
  REGION 1..8: note = an artificially synthesised sequence
TKGRSASP

SEQ ID NO: 713 - protein, 8 aa, synthetic construct
  REGION 1..8: note = an artificially synthesised sequence
TKGRSAHP

SEQ ID NO: 714 - protein, 8 aa, synthetic construct
  REGION 1..8: note = an artificially synthesised sequence
TKGRSANA

SEQ ID NO: 715 - protein, 8 aa, synthetic construct
  REGION 1..8: note = an artificially synthesised sequence
TKGRSANF

SEQ ID NO: 716 - protein, 8 aa, synthetic construct
  REGION 1..8: note = an artificially synthesised sequence
TKGRSANY

SEQ ID NO: 717 - protein, 8 aa, synthetic construct
  REGION 1..8: note = an artificially synthesised sequence
TKGRSANV

SEQ ID NO: 718 - protein, 8 aa, synthetic construct
  REGION 1..8: note = an artificially synthesised sequence
TKGRSANH

SEQ ID NO: 719 - protein, 8 aa, synthetic construct
  REGION 1..8: note = an artificially synthesised sequence
TSGRSAVY

SEQ ID NO: 720 - protein, 8 aa, synthetic construct
  REGION 1..8: note = an artificially synthesised sequence
TSGRSAVV

SEQ ID NO: 721 - protein, 8 aa, synthetic construct
  REGION 1..8: note = an artificially synthesised sequence
TSGRSAVH

SEQ ID NO: 722 - protein, 8 aa, synthetic construct
  REGION 1..8: note = an artificially synthesised sequence
TSGRSAIY

SEQ ID NO: 723 - protein, 8 aa, synthetic construct
  REGION 1..8: note = an artificially synthesised sequence
TSGRSAIV

SEQ ID NO: 724 - protein, 8 aa, synthetic construct
  REGION 1..8: note = an artificially synthesised sequence
TSGRSAIH

SEQ ID NO: 725 - protein, 8 aa, synthetic construct
  REGION 1..8: note = an artificially synthesised sequence
TSGRSASY

SEQ ID NO: 726 - protein, 8 aa, synthetic construct
  REGION 1..8: note = an artificially synthesised sequence
TSGRSASV

SEQ ID NO: 727 - protein, 8 aa, synthetic construct
  REGION 1..8: note = an artificially synthesised sequence
TSGRSASH

SEQ ID NO: 728 - protein, 8 aa, synthetic construct
  REGION 1..8: note = an artificially synthesised sequence
TSGRSAHY

SEQ ID NO: 729 - protein, 8 aa, synthetic construct
  REGION 1..8: note = an artificially synthesised sequence
TSGRSAHV

SEQ ID NO: 730 - protein, 8 aa, synthetic construct
  REGION 1..8: note = an artificially synthesised sequence
TSGRSAHH

SEQ ID NO: 731 - protein, 8 aa, synthetic construct
  REGION 1..8: note = an artificially synthesised sequence
PSGRSEVP

SEQ ID NO: 732 - protein, 8 aa, synthetic construct
  REGION 1..8: note = an artificially synthesised sequence
PSGRSAEP

SEQ ID NO: 733 - protein, 8 aa, synthetic construct
  REGION 1..8: note = an artificially synthesised sequence
PSGRSAGP

SEQ ID NO: 734 - protein, 8 aa, synthetic construct
  REGION 1..8: note = an artificially synthesised sequence
ASGRSENA

SEQ ID NO: 735 - protein, 8 aa, synthetic construct
  REGION 1..8: note = an artificially synthesised sequence
ASGRSAEA

SEQ ID NO: 736 - protein, 8 aa, synthetic construct
  REGION 1..8: note = an artificially synthesised sequence
ASGRSAGA

SEQ ID NO: 737 - protein, 8 aa, synthetic construct
  REGION 1..8: note = an artificially synthesised sequence
GTGRSATP

SEQ ID NO: 738 - protein, 8 aa, synthetic construct
  REGION 1..8: note = an artificially synthesised sequence
GSGRSATY

SEQ ID NO: 739 - protein, 8 aa, synthetic construct
  REGION 1..8: note = an artificially synthesised sequence
GSGRSATV

SEQ ID NO: 740 - protein, 8 aa, synthetic construct
  REGION 1..8: note = an artificially synthesised sequence
GSGRSATH

SEQ ID NO: 741 - protein, 8 aa, synthetic construct
  REGION 1..8: note = an artificially synthesised sequence
GTGRSATY

SEQ ID NO: 742 - protein, 8 aa, synthetic construct
  REGION 1..8: note = an artificially synthesised sequence
GTGRSATV

SEQ ID NO: 743 - protein, 8 aa, synthetic construct
  REGION 1..8: note = an artificially synthesised sequence
GTGRSATH

SEQ ID NO: 744 - protein, 8 aa, synthetic construct
  REGION 1..8: note = an artificially synthesised sequence
GSGRSETP

SEQ ID NO: 745 - protein, 8 aa, synthetic construct
  REGION 1..8: note = an artificially synthesised sequence
GTGRSETP

SEQ ID NO: 746 - protein, 8 aa, synthetic construct
  REGION 1..8: note = an artificially synthesised sequence
GSGRSETY

SEQ ID NO: 747 - protein, 8 aa, synthetic construct
  REGION 1..8: note = an artificially synthesised sequence
GSGRSETV

SEQ ID NO: 748 - protein, 8 aa, synthetic construct
  REGION 1..8: note = an artificially synthesised sequence
GSGRSETH

SEQ ID NO: 749 - protein, 8 aa, synthetic construct
  REGION 1..8: note = an artificially synthesised sequence
YTGRSAVP

SEQ ID NO: 750 - protein, 8 aa, synthetic construct
  REGION 1..8: note = an artificially synthesised sequence
YSGRSAVY

SEQ ID NO: 751 - protein, 8 aa, synthetic construct
  REGION 1..8: note = an artificially synthesised sequence
YSGRSAVV

SEQ ID NO: 752 - protein, 8 aa, synthetic construct
  REGION 1..8: note = an artificially synthesised sequence
YSGRSAVH

SEQ ID NO: 753 - protein, 8 aa, synthetic construct
  REGION 1..8: note = an artificially synthesised sequence
YTGRSAVY

SEQ ID NO: 754 - protein, 8 aa, synthetic construct
  REGION 1..8: note = an artificially synthesised sequence
YTGRSAVV

SEQ ID NO: 755 - protein, 8 aa, synthetic construct
  REGION 1..8: note = an artificially synthesised sequence
YTGRSAVH

SEQ ID NO: 756 - protein, 8 aa, synthetic construct
  REGION 1..8: note = an artificially synthesised sequence
YSGRSEVP

SEQ ID NO: 757 - protein, 8 aa, synthetic construct
  REGION 1..8: note = an artificially synthesised sequence
YTGRSEVP

SEQ ID NO: 758 - protein, 8 aa, synthetic construct
  REGION 1..8: note = an artificially synthesised sequence
YSGRSEVY

SEQ ID NO: 759 - protein, 8 aa, synthetic construct
  REGION 1..8: note = an artificially synthesised sequence
YSGRSEVV

SEQ ID NO: 760 - protein, 8 aa, synthetic construct
  REGION 1..8: note = an artificially synthesised sequence
YSGRSEVH

SEQ ID NO: 761 - protein, 8 aa, synthetic construct
  REGION 1..8: note = an artificially synthesised sequence
YTGRSAVP

SEQ ID NO: 762 - protein, 8 aa, synthetic construct
  REGION 1..8: note = an artificially synthesised sequence
YSGRSAVY

SEQ ID NO: 763 - protein, 8 aa, synthetic construct
  REGION 1..8: note = an artificially synthesised sequence
YSGRSAVV

SEQ ID NO: 764 - protein, 8 aa, synthetic construct
  REGION 1..8: note = an artificially synthesised sequence
YSGRSAVH

SEQ ID NO: 765 - protein, 8 aa, synthetic construct
  REGION 1..8: note = an artificially synthesised sequence
YTGRSAVY

SEQ ID NO: 766 - protein, 8 aa, synthetic construct
  REGION 1..8: note = an artificially synthesised sequence
YTGRSAVV

SEQ ID NO: 767 - protein, 8 aa, synthetic construct
  REGION 1..8: note = an artificially synthesised sequence
YTGRSAVH

SEQ ID NO: 768 - protein, 12 aa, synthetic construct
  REGION 1..12: note = an artificially synthesised sequence
TSTSGRSANPRG

SEQ ID NO: 769 - protein, 12 aa, synthetic construct
  REGION 1..12: note = an artificially synthesised sequence
TSTSGRSANPAG

SEQ ID NO: 770 - protein, 12 aa, synthetic construct
  REGION 1..12: note = an artificially synthesised sequence
TSTSGRSANPHG

SEQ ID NO: 771 - protein, 12 aa, synthetic construct
  REGION 1..12: note = an artificially synthesised sequence
TSTSGRSANPIG

SEQ ID NO: 772 - protein, 12 aa, synthetic construct
  REGION 1..12: note = an artificially synthesised sequence
TSTSGRSANPLG

SEQ ID NO: 773 - protein, 12 aa, synthetic construct
  REGION 1..12: note = an artificially synthesised sequence
TSTSGRSANPSG

SEQ ID NO: 774 - protein, 12 aa, synthetic construct
  REGION 1..12: note = an artificially synthesised sequence
ISTSGRSANPIG

SEQ ID NO: 775 - protein, 12 aa, synthetic construct
  REGION 1..12: note = an artificially synthesised sequence
YSTSGRSANPIG

SEQ ID NO: 776 - protein, 12 aa, synthetic construct
  REGION 1..12: note = an artificially synthesised sequence
TSYSGRSAVPAG

SEQ ID NO: 777 - protein, 12 aa, synthetic construct
  REGION 1..12: note = an artificially synthesised sequence
TSPSGRSANIAG

SEQ ID NO: 778 - protein, 12 aa, synthetic construct
  REGION 1..12: note = an artificially synthesised sequence
TSPSGRSANFAG

SEQ ID NO: 779 - protein, 12 aa, synthetic construct
  REGION 1..12: note = an artificially synthesised sequence
TSPTGRSANPAG

SEQ ID NO: 780 - protein, 12 aa, synthetic construct
  REGION 1..12: note = an artificially synthesised sequence
TSPSGRSAIPAG

SEQ ID NO: 781 - protein, 12 aa, synthetic construct
  REGION 1..12: note = an artificially synthesised sequence
TSYTGRSANPAG

SEQ ID NO: 782 - protein, 12 aa, synthetic construct
  REGION 1..12: note = an artificially synthesised sequence
TSYSGRSAIPAG

SEQ ID NO: 783 - protein, 12 aa, synthetic construct
  REGION 1..12: note = an artificially synthesised sequence
TSISGRSANYAG

SEQ ID NO: 784 - protein, 12 aa, synthetic construct
  REGION 1..12: note = an artificially synthesised sequence
TSPSGRSAGPAG

SEQ ID NO: 785 - protein, 12 aa, synthetic construct
  REGION 1..12: note = an artificially synthesised sequence
TSYTGRSAVPAG

SEQ ID NO: 786 - protein, 12 aa, synthetic construct
  REGION 1..12: note = an artificially synthesised sequence
TSYTGRSAVYAG

SEQ ID NO: 787 - protein, 12 aa, synthetic construct
  REGION 1..12: note = an artificially synthesised sequence
TSYTGRSAVVAG

SEQ ID NO: 788 - protein, 12 aa, synthetic construct
  REGION 1..12: note = an artificially synthesised sequence
TSYTGRSAVHAG

SEQ ID NO: 789 - protein, 12 aa, synthetic construct
  REGION 1..12: note = an artificially synthesised sequence
TSYSGRSAVPHG

SEQ ID NO: 790 - protein, 12 aa, synthetic construct
  REGION 1..12: note = an artificially synthesised sequence
TSPSGRSANIHG

SEQ ID NO: 791 - protein, 12 aa, synthetic construct
  REGION 1..12: note = an artificially synthesised sequence
TSPSGRSANFHG

SEQ ID NO: 792 - protein, 12 aa, synthetic construct
  REGION 1..12: note = an artificially synthesised sequence
TSPTGRSANPHG

SEQ ID NO: 793 - protein, 12 aa, synthetic construct
  REGION 1..12: note = an artificially synthesised sequence
TSPSGRSAIPHG

SEQ ID NO: 794 - protein, 12 aa, synthetic construct
  REGION 1..12: note = an artificially synthesised sequence
TSYTGRSANPHG

SEQ ID NO: 795 - protein, 12 aa, synthetic construct
  REGION 1..12: note = an artificially synthesised sequence
TSYSGRSAIPHG

SEQ ID NO: 796 - protein, 12 aa, synthetic construct
  REGION 1..12: note = an artificially synthesised sequence
TSISGRSANYHG

SEQ ID NO: 797 - protein, 12 aa, synthetic construct
  REGION 1..12: note = an artificially synthesised sequence
TSPSGRSAGPHG

SEQ ID NO: 798 - protein, 12 aa, synthetic construct
  REGION 1..12: note = an artificially synthesised sequence
TSYTGRSAVPHG

SEQ ID NO: 799 - protein, 12 aa, synthetic construct
  REGION 1..12: note = an artificially synthesised sequence
TSYTGRSAVYHG

SEQ ID NO: 800 - protein, 12 aa, synthetic construct
  REGION 1..12: note = an artificially synthesised sequence
TSYTGRSAVVHG

SEQ ID NO: 801 - protein, 12 aa, synthetic construct
  REGION 1..12: note = an artificially synthesised sequence
TSYTGRSAVHHG

SEQ ID NO: 802 - protein, 12 aa, synthetic construct
  REGION 1..12: note = an artificially synthesised sequence
TSYSGRSAVPIG

SEQ ID NO: 803 - protein, 12 aa, synthetic construct
  REGION 1..12: note = an artificially synthesised sequence
TSPSGRSANIIG

SEQ ID NO: 804 - protein, 12 aa, synthetic construct
  REGION 1..12: note = an artificially synthesised sequence
TSPSGRSANFIG

SEQ ID NO: 805 - protein, 12 aa, synthetic construct
  REGION 1..12: note = an artificially synthesised sequence
TSPTGRSANPIG

SEQ ID NO: 806 - protein, 12 aa, synthetic construct
  REGION 1..12: note = an artificially synthesised sequence
TSPSGRSAIPIG

SEQ ID NO: 807 - protein, 12 aa, synthetic construct
  REGION 1..12: note = an artificially synthesised sequence
TSYTGRSANPIG

SEQ ID NO: 808 - protein, 12 aa, synthetic construct
  REGION 1..12: note = an artificially synthesised sequence
TSYSGRSAIPIG

SEQ ID NO: 809 - protein, 12 aa, synthetic construct
  REGION 1..12: note = an artificially synthesised sequence
TSISGRSANYIG

SEQ ID NO: 810 - protein, 12 aa, synthetic construct
  REGION 1..12: note = an artificially synthesised sequence
TSPSGRSAGPIG

SEQ ID NO: 811 - protein, 12 aa, synthetic construct
  REGION 1..12: note = an artificially synthesised sequence
TSYTGRSAVPIG

SEQ ID NO: 812 - protein, 12 aa, synthetic construct
  REGION 1..12: note = an artificially synthesised sequence
TSYTGRSAVYIG

SEQ ID NO: 813 - protein, 12 aa, synthetic construct
  REGION 1..12: note = an artificially synthesised sequence
TSYTGRSAVVIG

SEQ ID NO: 814 - protein, 12 aa, synthetic construct
  REGION 1..12: note = an artificially synthesised sequence
TSYTGRSAVHIG

SEQ ID NO: 815 - protein, 12 aa, synthetic construct
  REGION 1..12: note = an artificially synthesised sequence
TSYSGRSAVPLG

SEQ ID NO: 816 - protein, 12 aa, synthetic construct
  REGION 1..12: note = an artificially synthesised sequence
TSPSGRSANILG

SEQ ID NO: 817 - protein, 12 aa, synthetic construct
  REGION 1..12: note = an artificially synthesised sequence
TSPSGRSANFLG

SEQ ID NO: 818 - protein, 12 aa, synthetic construct
  REGION 1..12: note = an artificially synthesised sequence
TSPTGRSANPLG

SEQ ID NO: 819 - protein, 12 aa, synthetic construct
  REGION 1..12: note = an artificially synthesised sequence
TSPSGRSAIPLG

SEQ ID NO: 820 - protein, 12 aa, synthetic construct
  REGION 1..12: note = an artificially synthesised sequence
TSYTGRSANPLG

SEQ ID NO: 821 - protein, 12 aa, synthetic construct
  REGION 1..12: note = an artificially synthesised sequence
TSYSGRSAIPLG

SEQ ID NO: 822 - protein, 12 aa, synthetic construct
  REGION 1..12: note = an artificially synthesised sequence
TSISGRSANYLG

SEQ ID NO: 823 - protein, 12 aa, synthetic construct
  REGION 1..12: note = an artificially synthesised sequence
TSPSGRSAGPLG

SEQ ID NO: 824 - protein, 12 aa, synthetic construct
  REGION 1..12: note = an artificially synthesised sequence
TSYTGRSAVPLG

SEQ ID NO: 825 - protein, 12 aa, synthetic construct
  REGION 1..12: note = an artificially synthesised sequence
TSYTGRSAVYLG

SEQ ID NO: 826 - protein, 12 aa, synthetic construct
  REGION 1..12: note = an artificially synthesised sequence
TSYTGRSAVVLG

SEQ ID NO: 827 - protein, 12 aa, synthetic construct
  REGION 1..12: note = an artificially synthesised sequence
TSYTGRSAVHLG

SEQ ID NO: 828 - protein, 12 aa, synthetic construct
  REGION 1..12: note = an artificially synthesised sequence
TSYSGRSAVPSG

SEQ ID NO: 829 - protein, 12 aa, synthetic construct
  REGION 1..12: note = an artificially synthesised sequence
TSPSGRSANISG

SEQ ID NO: 830 - protein, 12 aa, synthetic construct
  REGION 1..12: note = an artificially synthesised sequence
TSPSGRSANFSG

SEQ ID NO: 831 - protein, 12 aa, synthetic construct
  REGION 1..12: note = an artificially synthesised sequence
TSPTGRSANPSG

SEQ ID NO: 832 - protein, 12 aa, synthetic construct
  REGION 1..12: note = an artificially synthesised sequence
TSPSGRSAIPSG

SEQ ID NO: 833 - protein, 12 aa, synthetic construct
  REGION 1..12: note = an artificially synthesised sequence
TSYTGRSANPSG

SEQ ID NO: 834 - protein, 12 aa, synthetic construct
  REGION 1..12: note = an artificially synthesised sequence
TSYSGRSAIPSG

SEQ ID NO: 835 - protein, 12 aa, synthetic construct
  REGION 1..12: note = an artificially synthesised sequence
TSISGRSANYSG

SEQ ID NO: 836 - protein, 12 aa, synthetic construct
  REGION 1..12: note = an artificially synthesised sequence
TSPSGRSAGPSG

SEQ ID NO: 837 - protein, 12 aa, synthetic construct
  REGION 1..12: note = an artificially synthesised sequence
TSYTGRSAVPSG

SEQ ID NO: 838 - protein, 12 aa, synthetic construct
  REGION 1..12: note = an artificially synthesised sequence
TSYTGRSAVYSG

SEQ ID NO: 839 - protein, 12 aa, synthetic construct
  REGION 1..12: note = an artificially synthesised sequence
TSYTGRSAVVSG

SEQ ID NO: 840 - protein, 12 aa, synthetic construct
  REGION 1..12: note = an artificially synthesised sequence
TSYTGRSAVHSG

SEQ ID NO: 841 - protein, 12 aa, synthetic construct
  REGION 1..12: note = an artificially synthesised sequence
ISYSGRSAVPIG

SEQ ID NO: 842 - protein, 12 aa, synthetic construct
  REGION 1..12: note = an artificially synthesised sequence
ISPSGRSANIIG

SEQ ID NO: 843 - protein, 12 aa, synthetic construct
  REGION 1..12: note = an artificially synthesised sequence
ISPSGRSANFIG

SEQ ID NO: 844 - protein, 12 aa, synthetic construct
  REGION 1..12: note = an artificially synthesised sequence
ISPTGRSANPIG

SEQ ID NO: 845 - protein, 12 aa, synthetic construct
  REGION 1..12: note = an artificially synthesised sequence
ISPSGRSAIPIG

SEQ ID NO: 846 - protein, 12 aa, synthetic construct
  REGION 1..12: note = an artificially synthesised sequence
ISYTGRSANPIG

SEQ ID NO: 847 - protein, 12 aa, synthetic construct
  REGION 1..12: note = an artificially synthesised sequence
ISYSGRSAIPIG

SEQ ID NO: 848 - protein, 12 aa, synthetic construct
  REGION 1..12: note = an artificially synthesised sequence
ISISGRSANYIG

SEQ ID NO: 849 - protein, 12 aa, synthetic construct
  REGION 1..12: note = an artificially synthesised sequence
ISPSGRSAGPIG

SEQ ID NO: 850 - protein, 12 aa, synthetic construct
  REGION 1..12: note = an artificially synthesised sequence
ISYTGRSAVPIG

SEQ ID NO: 851 - protein, 12 aa, synthetic construct
  REGION 1..12: note = an artificially synthesised sequence
ISYTGRSAVYIG

SEQ ID NO: 852 - protein, 12 aa, synthetic construct
  REGION 1..12: note = an artificially synthesised sequence
ISYTGRSAVVIG

SEQ ID NO: 853 - protein, 12 aa, synthetic construct
  REGION 1..12: note = an artificially synthesised sequence
ISYTGRSAVHIG

SEQ ID NO: 854 - protein, 12 aa, synthetic construct
  REGION 1..12: note = an artificially synthesised sequence
YSYSGRSAVPIG

SEQ ID NO: 855 - protein, 12 aa, synthetic construct
  REGION 1..12: note = an artificially synthesised sequence
YSPSGRSANIIG

SEQ ID NO: 856 - protein, 12 aa, synthetic construct
  REGION 1..12: note = an artificially synthesised sequence
YSPSGRSANFIG

SEQ ID NO: 857 - protein, 12 aa, synthetic construct
  REGION 1..12: note = an artificially synthesised sequence
YSPTGRSANPIG

SEQ ID NO: 858 - protein, 12 aa, synthetic construct
  REGION 1..12: note = an artificially synthesised sequence
YSPSGRSAIPIG

SEQ ID NO: 859 - protein, 12 aa, synthetic construct
  REGION 1..12: note = an artificially synthesised sequence
YSYTGRSANPIG

SEQ ID NO: 860 - protein, 12 aa, synthetic construct
  REGION 1..12: note = an artificially synthesised sequence
YSYSGRSAIPIG

SEQ ID NO: 861 - protein, 12 aa, synthetic construct
  REGION 1..12: note = an artificially synthesised sequence
YSISGRSANYIG

SEQ ID NO: 862 - protein, 12 aa, synthetic construct
  REGION 1..12: note = an artificially synthesised sequence
YSPSGRSAGPIG

SEQ ID NO: 863 - protein, 12 aa, synthetic construct
  REGION 1..12: note = an artificially synthesised sequence
YSYTGRSAVPIG

SEQ ID NO: 864 - protein, 12 aa, synthetic construct
  REGION 1..12: note = an artificially synthesised sequence
YSYTGRSAVYIG

SEQ ID NO: 865 - protein, 12 aa, synthetic construct
  REGION 1..12: note = an artificially synthesised sequence
YSYTGRSAVVIG

SEQ ID NO: 866 - protein, 12 aa, synthetic construct
  REGION 1..12: note = an artificially synthesised sequence
YSYTGRSAVHIG

SEQ ID NO: 867 - protein, 12 aa, synthetic construct
  REGION 1..12: note = an artificially synthesised sequence
TSYTGRSAVPRG

SEQ ID NO: 868 - protein, 12 aa, synthetic construct
  REGION 1..12: note = an artificially synthesised sequence
TSYSGRSAVVRG

SEQ ID NO: 869 - protein, 12 aa, synthetic construct
  REGION 1..12: note = an artificially synthesised sequence
TSYTGRSAVYRG

SEQ ID NO: 870 - protein, 12 aa, synthetic construct
  REGION 1..12: note = an artificially synthesised sequence
TSYTGRSAVHRG

SEQ ID NO: 871 - protein, 306 aa, synthetic construct
  REGION 1..306: note = an artificially synthesised sequence
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 872 - protein, 197 aa, synthetic construct
  REGION 1..197: note = an artificially synthesised sequence
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 873 - protein, 12 aa, synthetic construct
  REGION 1..12: note = an artificially synthesised sequence
TSTSGRSANPRG

SEQ ID NO: 874 - protein, 550 aa, synthetic construct
  REGION 1..550: note = an artificially synthesised sequence
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGSGGSGGSTSTSGRSANPRGGGSGGSGGSEIVMTQSPATLSVSPGERATLSCRASQSISINLHWYQQKPGQAPRLLIYFASQSISGIPARFSGSGSGTEFTLTISSLQSEDFAVYYCQQSNSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 875 - protein, 683 aa, synthetic construct
  REGION 1..683: note = an artificially synthesised sequence
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSTSTSGRSANPRGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 876 - protein, 214 aa, synthetic construct
  REGION 1..214: note = an artificially synthesised sequence
EIVMTQSPATLSVSPGERATLSCRASQSISINLHWYQQKPGQAPRLLIYFASQSISGIPARFSGSGSGTEFTLTISSLQSEDFAVYYCQQSNSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 877 - protein, 30 aa, synthetic construct
  REGION 1..30: note = an artificially synthesised sequence
GGSGGSGGSTSTSGRSANPRGGGSGGSGGS

SEQ ID NO: 878 - protein, 118 aa, synthetic construct
  REGION 1..118: note = an artificially synthesised sequence
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSS

SEQ ID NO: 879 - protein, 18 aa, synthetic construct
  REGION 1..18: note = an artificially synthesised sequence
GGSTSTSGRSANPRGGGS

SEQ ID NO: 880 - protein, 1009 aa, synthetic construct
  REGION 1..1009: note = an artificially synthesised sequence
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVGGGGSGGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSTSTSGRSANPRGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 881 - protein, 1009 aa, synthetic construct
  REGION 1..1009: note = an artificially synthesised sequence
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNRYTQKSLSLSPGGSTSTSGRSANPRGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 882 - protein, 223 aa, synthetic construct
  REGION 1..223: note = an artificially synthesised sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 883 - protein, 447 aa, synthetic construct
  REGION 1..447: note = an artificially synthesised sequence
DIQMTQSSSSFSVSLGDRVTITCKASEDIYNRLAWYQQKPGNAPRLLISGATSLETGVPSRFSGSGSGKDYTLSITSLQTEDVATYYCQQYWSTPYTFGGGTKLEVKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRCEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 884 - protein, 683 aa, synthetic construct
  REGION 1..683: note = an artificially synthesised sequence
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 885 - protein, 1009 aa, synthetic construct
  REGION 1..1009: note = an artificially synthesised sequence
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 886 - protein, 1009 aa, synthetic construct
  REGION 1..1009: note = an artificially synthesised sequence
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNRYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 887 - protein, 214 aa, synthetic construct
  REGION 1..214: note = an artificially synthesised sequence
DIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 888 - protein, 1010 aa, synthetic construct
  REGION 1..1010: note = an artificially synthesised sequence
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNRYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 889 - protein, 119 aa, synthetic construct
  REGION 1..119: note = an artificially synthesised sequence
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSS

SEQ ID NO: 890 - protein, 219 aa, synthetic construct
  REGION 1..219: note = an artificially synthesised sequence
QSVLTQPPSVSGAPGQRVTISCSGSRSNIGSNTVKWYQQLPGTAPKLLIYYNDQRPSGVPDRFSGSKSGTSASLAITGLQAEDEADYYCQSYDRYTHPALLFGTGTKVTVLGRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 891 - protein, 1006 aa, synthetic construct
  REGION 1..1006: note = an artificially synthesised sequence
QVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNRYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 892 - protein, 115 aa, synthetic construct [sequence omitted]

SEQ ID NO: 893 - protein, 5 aa, synthetic construct
  REGION 1..5: note = an artificially synthesised sequence
GGGGA

SEQ ID NO: 894 - protein, 5 aa, synthetic construct
  REGION 1..5: note = an artificially synthesised sequence
GGGGE

SEQ ID NO: 895 - protein, 4 aa, synthetic construct
  REGION 1..4: note = an artificially synthesised sequence
GGGA

SEQ ID NO: 896 - protein, 4 aa, synthetic construct
  REGION 1..4: note = an artificially synthesised sequence
GGGE

SEQ ID NO: 897 - protein, 4 aa, synthetic construct
  REGION 1..4: note = an artificially synthesised sequence
QQQG

SEQ ID NO: 898 - protein, 5 aa, synthetic construct
  REGION 1..5: note = an artificially synthesised sequence
QQQQG

SEQ ID NO: 899 - protein, 4 aa, synthetic construct
  REGION 1..4: note = an artificially synthesised sequence
SSSG

SEQ ID NO: 900 - protein, 5 aa, synthetic construct
  REGION 1..5: note = an artificially synthesised sequence
SSSSG

SEQ ID NO: 901 - protein, 338 aa, synthetic construct
  REGION 1..338: note = an artificially synthesised sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 902 - protein, 523 aa, synthetic construct
  REGION 1..523: note = an artificially synthesised sequence
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 903 - protein, 18 aa, synthetic construct
  REGION 1..18: note = an artificially synthesised sequence
GGSGGSGGSGGSGGSGGS

SEQ ID NO: 904 - protein, 1009 aa, synthetic construct
  REGION 1..1009: note = an artificially synthesised sequence
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 905 - protein, 338 aa, synthetic construct
  REGION 1..338: note = an artificially synthesised sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 906 - protein, 214 aa, synthetic construct
  REGION 1..214: note = an artificially synthesised sequence
EIVMTQSPATLSVSPGERATLSCRASQSISINLHWYQQKPGQAPRLLIYFASQSISGIPARFSGSGSGTEFTLTISSLQSEDFAVYYCQQSNSFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 907 - protein, 1009 aa, synthetic construct
  REGION 1..1009: note = an artificially synthesised sequence
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 908 - protein, 338 aa, synthetic construct
  REGION 1..338: note = an artificially synthesised sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 909 - protein, 1009 aa, synthetic construct
  REGION 1..1009: note = an artificially synthesised sequence
QVQLVQSGAEVKKPGASVKVSCKASGYTFTDYYLHWVRQAPGQGLEWMGWIDPENGDTEYAPKFQGRVTMTTDTSTSTAYMELRSLRSDDTAVYYCNANKELRYFDVWGQGTMVTVSSTSTSGRSANPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 910 - protein, 338 aa, synthetic construct
  REGION 1..338: note = an artificially synthesised sequence
ASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 911 - protein, 146 aa, synthetic construct
  REGION 1..146: note = an artificially synthesised sequence
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 912 - protein, 216 aa, synthetic construct
  REGION 1..216: note = an artificially synthesised sequence
QAALTQPASVSGSPGQSITISCTGASGDVGAYNFVSWYQQHPGKAPKLIIYDVNKRPSGVSNRFSGSKSGNTASLTISGLQAEDEADYYCSSYTSTFSVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 913 - protein, 635 aa, synthetic construct
  REGION 1..635: note = an artificially synthesised sequence
EVQLVESGGGVVRPGGSLRLSCAASGFTFDDYGMNWVRQAPGKGLEWVSGVNWNGGTRDYAASVKGRFTISRDNAKNSLYLQMNSLRAEDTALYHCARGWYSGAAWNMGYWGRGTLVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSTSTSGRSANPRGGGSAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 914 - protein, 121 aa, synthetic construct
  REGION 1..121: note = an artificially synthesised sequence
EVQLVESGGGVVRPGGSLRLSCAASGFTFDDYGMNWVRQAPGKGLEWVSGVNWNGGTRDYAASVKGRFTISRDNAKNSLYLQMNSLRAEDTALYHCARGWYSGAAWNMGYWGRGTLVTVSS

SEQ ID NO: 915 - protein, 216 aa, synthetic construct
  REGION 1..216: note = an artificially synthesised sequence
QAALTQPASVSGSPGQSITISCTGASGDVGAYNFVSWYQQHPGKAPKLIIYDVNKRPSGVSNRFSGSKSGNTASLTISGLQAEDEADYYCSSYTSTFSVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTESS

SEQ ID NO: 916 - protein, 635 aa, synthetic construct
  REGION 1..635: note = an artificially synthesised sequence
EVQLVESGGGVVRPGGSLRLSCAASGFTFDDYGMNWVRQAPGKGLEWVSGVNWNGGTRDYAASVKGRFTISRDNAKNSLYLQMNSLRAEDTALYHCARGWYSGAAWNMGYWGRGTLVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSTSTSGRSANPRGGGSAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 917 - protein, 623 aa, synthetic construct
  REGION 1..623: note = an artificially synthesised sequence
EVQLVESGGGVVRPGGSLRLSCAASGFTFDDYGMNWVRQAPGKGLEWVSGVNWNGGTRDYAASVKGRFTISRDNAKNSLYLQMNSLRAEDTALYHCARGWYSGAAWNMGYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 918 - protein, 141 aa, synthetic construct
  REGION 1..141: note = an artificially synthesised sequence
APTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFAQSIISTLTHHHHHHHH

SEQ ID NO: 919 - protein, 637 aa, synthetic construct
  REGION 1..637: note = an artificially synthesised sequence
QVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARWRGDGYYAYFDVWGAGTTVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTGGGGSGGGGSCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQEGNVFSCSVMHEALHNHYTQKSLSLSPGGGGSGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFAQSIISTLTHHHHHHHH

SEQ ID NO: 920 - protein, 218 aa, synthetic construct
  REGION 1..218: note = an artificially synthesised sequence
DIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 921 - protein, 637 aa, synthetic construct
  REGION 1..637: note = an artificially synthesised sequence
QVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCARWRGDGYYAYFDVWGAGTTVTVSSGGGSGGGSGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTGGGGSGGGGSCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQEGNVFSCSVMHEALHNHYTQKSLSLSPGGGGSGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFAQSIISTLTHHHHHHHH

SEQ ID NO: 922 - protein, 635 aa, synthetic construct
  REGION 1..635: note = an artificially synthesised sequence
QVQLVQSGGGVVQPGRSLRLSCAASGFTFSNYAMNWVRQAPGKGLEWVTLISYDGSQKYYADSVKGRFTTSRDNSKNTLYLQMNSLRAEDTAVYYCARDSTTLGAFDVWGQGTMVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTGGGGSGGGGSCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQEGNVFSCSVMHEALHNHYTQKSLSLSPGGGGSGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFAQSIISTLTHHHHHHHH

SEQ ID NO: 923 - protein, 214 aa, synthetic construct
  REGION 1..214: note = an artificially synthesised sequence
SYELTQPPSLSVSPGQTATISCSGDAFPRKFAYWYQQKSGQAPVLVIYEDTRRPSGIPERFSGSSSGTMATLTISGAQVEDEADYFCYSTDTTGTHRVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 924 - protein, 635 aa, synthetic construct
  REGION 1..635: note = an artificially synthesised sequence
QVQLVQSGGGVVQPGRSLRLSCAASGFTFSNYAMNWVRQAPGKGLEWVTLISYDGSQKYYADSVKGRFTTSRDNSKNTLYLQMNSLRAEDTAVYYCARDSTTLGAFDVWGQGTMVTVSSGGGSGGGSGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTGGGGSGGGGSCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQEGNVFSCSVMHEALHNHYTQKSLSLSPGGGGSGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFAQSIISTLTHHHHHHHH

SEQ ID NO: 925 - protein, 632 aa, synthetic construct
  REGION 1..632: note = an artificially synthesised sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSGGGSGGGSGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTGGGGSGGGGSCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQEGNVFSCSVMHEALHNHYTQKSLSLSPGGGGSGGGGSGGGGSGGGGSGGGGSAPTSSSTKKTQLQLEHLLLDLQMILNGINNYKNPKLTRMLTFKFYMPKKATELKHLQCLEEELKPLEEVLNLAQSKNFHLRPRDLISDINVIVLELKGSETTFMCEYADETATIVEFLNRWITFAQSIISTLTHHHHHHHH

SEQ ID NO: 926 - protein, 214 aa, synthetic construct
  REGION 1..214: note = an artificially synthesised sequence
DIQMTQSSSSFSVSLGDRVTITCKASEDIYNRLAWYQQKPGNAPRLLISGATSLETGVPSRFSGSGSGKDYTLSITSLQTEDVATYYCQQYWSTPYTFGGGTKLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 927 - protein, 25 aa, synthetic construct
  REGION 1..25: note = an artificially synthesised sequence
GGGGSGGGGSGGGGSGGGGSGGGGS

SEQ ID NO: 928 - protein, 12 aa, synthetic construct
  REGION 1..12: note = an artificially synthesised sequence
GGGSGGGSGGGS

SEQ ID NO: 929 - protein, 635 aa, synthetic construct
  REGION 1..635: note = an artificially synthesised sequence
EVQLVESGGGVVRPGGSLRLSCAASGFTFDDYGMNWVRQAPGKGLEWVSGVNWNGGTRDYAASVKGRFTISRDNAKNSLYLQMNSLRAEDTALYHCARGWYSGAAWNMGYWGRGTLVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCGGGGSGGGGSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSTSTSGRSANPRGGGSAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 930 - protein, 635 aa, synthetic construct
  REGION 1..635: note = an artificially synthesised sequence
EVQLVESGGGVVRPGGSLRLSCAASGFTFDDYGMNWVRQAPGKGLEWVSGVNWNGGTRDYAASVKGRFTISRDNAKNSLYLQMNSLRAEDTALYHCARGWYSGAAWNMGYWGRGTLVTVSSTSTSGRSANPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSTSTSGRSANPRGGGSAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 931 - protein, 121 aa, synthetic construct [sequence omitted]

SEQ ID NO: 932 - protein, 338 aa, synthetic construct
  REGION 1..338: note = an artificially synthesised sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTGGGGSGGGGSCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQEGNVFSCSVMHEALHNHYTQKSLSLSP

SEQ ID NO: 933 - protein, 119 aa, synthetic construct
  REGION 1..119: note = an artificially synthesised sequence
QVQLVQSGGGVVQPGRSLRLSCAASGFTFSNYAMNWVRQAPGKGLEWVTLISYDGSQKYYADSVKGRFTTSRDNSKNTLYLQMNSLRAEDTAVYYCARDSTTLGAFDVWGQGTMVTVSS

SEQ ID NO: 934 - protein, 116 aa, synthetic construct
  REGION 1..116: note = an artificially synthesised sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSS

SEQ ID NO: 935 - protein, 25 aa, synthetic construct
  REGION 1..25: note = an artificially synthesised sequence
EPKSCGGGGSGGGGSDKTHTCPPCP

SEQ ID NO: 936 - protein, 5 aa, synthetic construct
  REGION 1..5: note = an artificially synthesised sequence
EPKSC

SEQ ID NO: 937 - protein, 25 aa, synthetic construct
  REGION 1..25: note = an artificially synthesised sequence
GGGGSGGGGSEPKSCDKTHTCPPCP

SEQ ID NO: 938 - protein, 25 aa, synthetic construct
  REGION 1..25: note = an artificially synthesised sequence
EPKSCDKTHTGGGGSGGGGSCPPCP

SEQ ID NO: 939 - protein, 306 aa, synthetic construct; identical to SEQ ID NO: 871
  REGION 1..306: note = an artificially synthesised sequence

SEQ ID NO: 940 - protein, 197 aa, synthetic construct; identical to SEQ ID NO: 872
  REGION 1..197: note = an artificially synthesised sequence

SEQ ID NO: 941 - protein, 12 aa, synthetic construct
  REGION 1..12: note = an artificially synthesised sequence
TSTSGRSANPRG

SEQ ID NO: 942 - protein, 550 aa, synthetic construct; identical to SEQ ID NO: 874
  REGION 1..550: note = an artificially synthesised sequence

SEQ ID NO: 943 - protein, 683 aa, synthetic construct; identical to SEQ ID NO: 875
  REGION 1..683: note = an artificially synthesised sequence

SEQ ID NO: 944 - protein, 214 aa, synthetic construct; identical to SEQ ID NO: 876
  REGION 1..214: note = an artificially synthesised sequence

SEQ ID NO: 945 - protein, 30 aa, synthetic construct; identical to SEQ ID NO: 877
  REGION 1..30: note = an artificially synthesised sequence

SEQ ID NO: 946 - protein, 118 aa, synthetic construct; identical to SEQ ID NO: 878
  REGION 1..118: note = an artificially synthesised sequence

SEQ ID NO: 947 - protein, 18 aa, synthetic construct
  REGION 1..18: note = an artificially synthesised sequence
GGSTSTSGRSANPRGGGS

SEQ ID NO: 948 - protein, 1009 aa, synthetic construct; identical to SEQ ID NO: 880
  REGION 1..1009: note = an artificially synthesised sequence

SEQ ID NO: 949 - protein, 1009 aa, synthetic construct; identical to SEQ ID NO: 881
  REGION 1..1009: note = an artificially synthesised sequence

SEQ ID NO: 950 - protein, 223 aa, synthetic construct; identical to SEQ ID NO: 882
  REGION 1..223: note = an artificially synthesised sequence

SEQ ID NO: 951 - protein, 447 aa, synthetic construct; identical to SEQ ID NO: 883
  REGION 1..447: note = an artificially synthesised sequence

SEQ ID NO: 952 - protein, 683 aa, synthetic construct; identical to SEQ ID NO: 884
  REGION 1..683: note = an artificially synthesised sequence

SEQ ID NO: 953 - protein, 1009 aa, synthetic construct; identical to SEQ ID NO: 885
  REGION 1..1009: note = an artificially synthesised sequence

SEQ ID NO: 954 - protein, 1009 aa, synthetic construct; identical to SEQ ID NO: 886
  REGION 1..1009: note = an artificially synthesised sequence

SEQ ID NO: 955 - protein, 214 aa, synthetic construct; identical to SEQ ID NO: 887
  REGION 1..214: note = an artificially synthesised sequence

SEQ ID NO: 956 - protein, 1010 aa, synthetic construct; identical to SEQ ID NO: 888
  REGION 1..1010: note = an artificially synthesised sequence

SEQ ID NO: 957 - protein, 119 aa, synthetic construct; identical to SEQ ID NO: 889
  REGION 1..119: note = an artificially synthesised sequence

SEQ ID NO: 958 - protein, 219 aa, synthetic construct; identical to SEQ ID NO: 890
  REGION 1..219: note = an artificially synthesised sequence

SEQ ID NO: 959 - protein, 1006 aa, synthetic construct; identical to SEQ ID NO: 891
  REGION 1..1006: note = an artificially synthesised sequence

SEQ ID NO: 960 - protein, 115 aa, synthetic construct [sequence omitted]

SEQ ID NO: 961 - protein, 338 aa, synthetic construct; identical to SEQ ID NO: 901
  REGION 1..338: note = an artificially synthesised sequence

SEQ ID NO: 962 - protein, 523 aa, synthetic construct; identical to SEQ ID NO: 902
  REGION 1..523: note = an artificially synthesised sequence

SEQ ID NO: 963 - protein, 18 aa, synthetic construct
  REGION 1..18: note = an artificially synthesised sequence
GGSGGSGGSGGSGGSGGS

SEQ ID NO: 964 - protein, 1009 aa, synthetic construct; identical to SEQ ID NO: 904
  REGION 1..1009: note = an artificially synthesised sequence

SEQ ID NO: 965 - protein, 338 aa, synthetic construct; identical to SEQ ID NO: 905
  REGION 1..338: note = an artificially synthesised sequence

SEQ ID NO: 966 - protein, 214 aa, synthetic construct; identical to SEQ ID NO: 906
  REGION 1..214: note = an artificially synthesised sequence

SEQ ID NO: 967 - protein, 1009 aa, synthetic construct; identical to SEQ ID NO: 907
  REGION 1..1009: note = an artificially synthesised sequence

SEQ ID NO: 968 - protein, 338 aa, synthetic construct; identical to SEQ ID NO: 908
  REGION 1..338: note = an artificially synthesised sequence

SEQ ID NO: 969 - protein, 1009 aa, synthetic construct; identical to SEQ ID NO: 909
  REGION 1..1009: note = an artificially synthesised sequence

SEQ ID NO: 970 - protein, 338 aa, synthetic construct; identical to SEQ ID NO: 910
  REGION 1..338: note = an artificially synthesised sequence

SEQ ID NO: 971 - protein, 146 aa, synthetic construct; identical to SEQ ID NO: 911
  REGION 1..146: note = an artificially synthesised sequence

SEQ ID NO: 972 - protein, 216 aa, synthetic construct; identical to SEQ ID NO: 912
  REGION 1..216: note = an artificially synthesised sequence

SEQ ID NO: 973 - protein, 635 aa, synthetic construct; identical to SEQ ID NO: 913
  REGION 1..635: note = an artificially synthesised sequence

SEQ ID NO: 974 - protein, 121 aa, synthetic construct; identical to SEQ ID NO: 914
  REGION 1..121: note = an artificially synthesised sequence

SEQ ID NO: 975 - protein, 216 aa, synthetic construct; identical to SEQ ID NO: 915
  REGION 1..216: note = an artificially synthesised sequence

SEQ ID NO: 976 - protein, 635 aa, synthetic construct; identical to SEQ ID NO: 916
  REGION 1..635: note = an artificially synthesised sequence

SEQ ID NO: 977 - protein, 623 aa, synthetic construct; identical to SEQ ID NO: 917
  REGION 1..623: note = an artificially synthesised sequence

SEQ ID NO: 978 - protein, 141 aa, synthetic construct; identical to SEQ ID NO: 918
  REGION 1..141: note = an artificially synthesised sequence

SEQ ID NO: 979 - protein, 637 aa, synthetic construct; identical to SEQ ID NO: 919
  REGION 1..637: note = an artificially synthesised sequence

SEQ ID NO: 980 - protein, 218 aa, synthetic construct; identical to SEQ ID NO: 920
  REGION 1..218: note = an artificially synthesised sequence

SEQ ID NO: 981 - protein, 637 aa, synthetic construct; identical to SEQ ID NO: 921
  REGION 1..637: note = an artificially synthesised sequence

SEQ ID NO: 982 - protein, 635 aa, synthetic construct; identical to SEQ ID NO: 922
  REGION 1..635: note = an artificially synthesised sequence

SEQ ID NO: 983 - protein, 214 aa, synthetic construct; identical to SEQ ID NO: 923
  REGION 1..214: note = an artificially synthesised sequence

SEQ ID NO: 984 - protein, 635 aa, synthetic construct; identical to SEQ ID NO: 924
  REGION 1..635: note = an artificially synthesised sequence

SEQ ID NO: 985 - protein, 632 aa, synthetic construct; identical to SEQ ID NO: 925
  REGION 1..632: note = an artificially synthesised sequence

SEQ ID NO: 986 - protein, 214 aa, synthetic construct; identical to SEQ ID NO: 926
  REGION 1..214: note = an artificially synthesised sequence

SEQ ID NO: 987 - protein, 25 aa, synthetic construct
  REGION 1..25: note = an artificially synthesised sequence
GGGGSGGGGSGGGGSGGGGSGGGGS

SEQ ID NO: 988 - protein, 12 aa, synthetic construct
  REGION 1..12: note = an artificially synthesised sequence
GGGSGGGSGGGS

SEQ ID NO: 989 - protein, 635 aa, synthetic construct; identical to SEQ ID NO: 929
  REGION 1..635: note = an artificially synthesised sequence

SEQ ID NO: 990 - protein, 635 aa, synthetic construct; identical to SEQ ID NO: 930
  REGION 1..635: note = an artificially synthesised sequence

SEQ ID NO: 991 - protein, 121 aa, synthetic construct [sequence omitted]

SEQ ID NO: 992 - protein, 338 aa, synthetic construct; identical to SEQ ID NO: 932
  REGION 1..338: note = an artificially synthesised sequence

SEQ ID NO: 993 - protein, 119 aa, synthetic construct; identical to SEQ ID NO: 933
  REGION 1..119: note = an artificially synthesised sequence

SEQ ID NO: 994 - protein, 116 aa, synthetic construct; identical to SEQ ID NO: 934
  REGION 1..116: note = an artificially synthesised sequence

SEQ ID NO: 995 - protein, 25 aa, synthetic construct
  REGION 1..25: note = an artificially synthesised sequence
EPKSCGGGGSGGGGSDKTHTCPPCP

SEQ ID NO: 996 - protein, 5 aa, synthetic construct
  REGION 1..5: note = an artificially synthesised sequence
EPKSC

SEQ ID NO: 997 - protein, 25 aa, synthetic construct
  REGION 1..25: note = an artificially synthesised sequence
GGGGSGGGGSEPKSCDKTHTCPPCP

SEQ ID NO: 998 - protein, 25 aa, synthetic construct
  REGION 1..25: note = an artificially synthesised sequence
EPKSCDKTHTGGGGSGGGGSCPPCP

SEQ ID NO: 999 - protein, 1010 aa, synthetic construct
  REGION 1..1010: note = an artificially synthesised sequence
EVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNRYTQKSLSLSPGGSTSTSGRSANPRGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 1000 - protein, 1010 aa, synthetic construct
  REGION 1..1010: note = an artificially synthesised sequence
QVQLVESGGGVVQPGRSLRLSCAASGFTFSSYGMHWVRQAPGKGLEWVAFIRYDGSNKYYADSVKGRFTISRDNSKNTLYLQMNSLRAEDTAVYYCKTHGSHDNWGQGTMVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNRYTQKSLSLSPGGGGSTSTSGRSANPRGGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 1001 - protein, 10 aa, synthetic construct
  REGION 1..10: note = an artificially synthesised sequence
GGGGSGGGGS

SEQ ID NO: 1002 - protein, 212 aa, synthetic construct
  REGION 1..212: note = an artificially synthesised sequence
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASENLYFQGDYKDDDDK

SEQ ID NO: 1003 - protein, 7 aa, synthetic construct
  REGION 1..7: note = an artificially synthesised sequence
ENLYFQG

SEQ ID NO: 1004 - protein, 8 aa, synthetic construct
  REGION 1..8: note = an artificially synthesised sequence
DYKDDDDK

SEQ ID NO: 1005 - protein, 985 aa, synthetic construct
  REGION 1..985: note = an artificially synthesised sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 1006 - protein, 328 aa, synthetic construct
  REGION 1..328: note = an artificially synthesised sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 1007 - protein, 985 aa, synthetic construct
  REGION 1..985: note = an artificially synthesised sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1008 - protein, 523 aa, synthetic construct
  REGION 1..523: note = an artificially synthesised sequence
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1009 - protein, 214 aa, synthetic construct
  REGION 1..214: note = an artificially synthesised sequence
DIVMTQSPLSLPVTPGEPASISCRASQSISINLHWYQQKPGQAPRLLIYFASQSISGVPSRFSGSGSGTDFTLTISSLQPEDFATYYCQQSNSFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1010 - protein, 458 aa, synthetic construct
  REGION 1..458: note = an artificially synthesised sequence
QVQLVQSGAEVKKPGSSVKVSCKASGGTFSDYYLHWVRQAPGQGLEWMGWIDPENGDTEYAPKFQGRVTITADESTSTAYMELSSLRSEDTAVYYCARNKELRYFDVWGQGTLVTVSSTSTSGRSANPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 1011 - protein, 118 aa, synthetic construct
  REGION 1..118: note = an artificially synthesised sequence
QVQLVQSGAEVKKPGSSVKVSCKASGGTFSDYYLHWVRQAPGQGLEWMGWIDPENGDTEYAPKFQGRVTITADESTSTAYMELSSLRSEDTAVYYCARNKELRYFDVWGQGTLVTVSS

SEQ ID NO: 1012 - protein, 1009 aa, synthetic construct
  REGION 1..1009: note = an artificially synthesised sequence
QVQLVQSGAEVKKPGSSVKVSCKASGGTFSDYYLHWVRQAPGQGLEWMGWIDPENGDTEYAPKFQGRVTITADESTSTAYMELSSLRSEDTAVYYCARNKELRYFDVWGQGTLVTVSSTSTSGRSANPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1013 - protein, 1009 aa, synthetic construct
  REGION 1..1009: note = an artificially synthesised sequence
QVQLVQSGAEVKKPGSSVKVSCKASGGTFSDYYLHWVRQAPGQGLEWMGWIDPENGDTEYAPKFQGRVTITADESTSTAYMELSSLRSEDTAVYYCARNKELRYFDVMGQGTLVTVSSTSTSGRSANPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1014 - protein, 1009 aa, synthetic construct
  REGION 1..1009: note = an artificially synthesised sequence
QVQLVQSGAEVKKPGSSVKVSCKASGGTFSDYYLHWVRQAPGQGLEWMGWIDPENGDTEYAPKFQGRVTITADESTSTAYMELSSLRSEDTAVYYCARNKELRYFDVLGQGTLVTVSSTSTSGRSANPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1015 - protein, 1009 aa, synthetic construct
  REGION 1..1009: note = an artificially synthesised sequence
QVQLVQSGAEVKKPGSSVKVSCKASGGTFSDYYLHWSRQAPGQGLEWMGWIDPENGDTEYAPKFQGRVTITADESTSTAYMELSSLRSEDTAVYYCARNKELRYIDVWGQGTLVTVSSTSTSGRSANPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSRNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1016 - protein, 214 aa, synthetic construct
  REGION 1..214: note = an artificially synthesised sequence
DIVMTQSPLSLPVTPGEPASISCRASQSISINLHWYQQKPGQAPRQLIAFASQSISGVPSRFSGSGSGTDFTLTISSLQPEDFATYYCQQSNSFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1017 - protein, 214 aa, synthetic construct
  REGION 1..214: note = an artificially synthesised sequence
DIVMTQSPLSLPVTPGEPASISCRASQSIVINLHWYQQKPGQAPRQLIAFASQSISGVPSRFSGSGSGTDFTLTISSLQPEDFATYYCQQSNSFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1018 - protein, 214 aa, synthetic construct
  REGION 1..214: note = an artificially synthesised sequence
DIVMTQSPLSLPVTPGEPASISCRASQSISINLHWYQQKPGQAPRLLIAFASQSISGVPSRFSGSGSGTDFTLTISSLQPEDFATYLCQQSNSFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1019 - protein, 214 aa, synthetic construct
  REGION 1..214: note = an artificially synthesised sequence
DIVMTQSPLSLPVTPGEPASISCRASQSIVINLHWYQQKPGQAPRLLIAFASQSISGVPSRFSGSGSGTDFTLTISSLQPEDFATYLCQQSNSFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 1020 - protein, 78 aa, synthetic construct
  REGION 1..78: note = an artificially synthesised sequence
VFPLSRTVRCTCISISNQPVNPRSLEKLEIIPASQFCPRVEIIATMKKKGEKRCLNPESKAIKNLLKAVSKERSKASP

SEQ ID NO: 1021 - protein, 216 aa, synthetic construct
  REGION 1..216: note = an artificially synthesised sequence
QSVLTQPPSASGTPGQRVTISCSGSSSNIGSNTVNWYQRLPGAAPQLLIYNNDQRPSGIPDRFSGSKSGTSGSLVISGLQSEDEADYYCASWDDSLNGRVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 1022 - protein, 561 aa, synthetic construct
  REGION 1..561: note = an artificially synthesised sequence
VFPLSRTVRCTCISISNQPVNPRSLEKLEIIPASQFCPRVEIIATMKKKGEKRCLNPESKAIKNLLKAVSKERSKASPGGGGSGGGGQVQLVESGGGVVQPGRSLRLSCAASGFTFSSFSITWIRQPPGKGLEWIGEITPMFGIANYAQKFQGQVTISADKSISTAYLQWSSLKASDTAMYYCARDGRFDVSDLLTDKPKVTINYNGMDVWGQGTLVTVSSTSGSGRSATPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQEGNVFSCSVMHEALHNHYTQKSLSLSP

SEQ ID NO: 1023 - protein, 216 aa, synthetic construct
  REGION 1..216: note = an artificially synthesised sequence
QSVLTQPPSASGTPGQRVTISCSGSSSNIGSNTVNWYQELPGAAPQLLIYNNDQRPSGIPDRFSGSKSGTSGSLVISGLQSEDEADYYCASWDDSLNGRVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 1024 - protein, 134 aa, synthetic construct
  REGION 1..134: note = an artificially synthesised sequence
QVQLVESGGGVVQPGRSLRLSCAASGFTFSSFSITWIRQPPGKGLEWIGEITPMFGIANYAQKFQGQVTISADKSISTAYLQWSSLKASDTAMYYCARDGRFDVSDLLTDKPKVTINYNGMDVWGQGTLVTVSS

SEQ ID NO: 1025 - protein, 9 aa, synthetic construct
  REGION 1..9: note = an artificially synthesised sequence
GGGGSGGGG

SEQ ID NO: 1026 - protein, 328 aa, synthetic construct
  REGION 1..328: note = an artificially synthesised sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQEGNVFSCSVMHEALHNHYTQKSLSLSP

SEQ ID NO: 1027 - protein, 12 aa, synthetic construct
  REGION 1..12: note = an artificially synthesised sequence
TSGSGRSATPRG

SEQ ID NO: 1028 - protein, 561 aa, synthetic construct
  REGION 1..561: note = an artificially synthesised sequence
VFPLSRTVRCTCISISNQPVNPRSLEKLEIIPASQFCPRVEIIATMKKKGEKRCLNPESKAIKNLLKAVSKERSKASPGGGGSGGGGQVQLVESGGGVVQPGRSLRLSCAASGFTFSSFSITWIRDPPGKGLEWIGEITPMFGIANYAQKFQGQVTISADKSISTAYLQWSSLKASDTAMYYCARDGRFDVSDLLTDKPKVTINYNGMDVWGQGTLVTVSSTSGSGRSATPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQEGNVFSCSVMHEALHNHYTQKSLSLSP

SEQ ID NO: 1029 - protein, 20 aa, synthetic construct
  REGION 1..20: note = an artificially synthesised sequence
GGGGSGGGGSGGGGSGGGGS

SEQ ID NO: 1030 - protein, 9 aa, synthetic construct
  REGION 1..9: note = an artificially synthesised sequence
KSKREKKDR

SEQ ID NO: 1031 - protein, 24 aa, synthetic construct
  REGION 1..24: note = an artificially synthesised sequence
GGGGSGGGGSGGGGSGGGGSRNLP

SEQ ID NO: 1032 - protein, 984 aa, synthetic construct
  REGION 1..984: note = an artificially synthesised sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1033 - protein, 522 aa, synthetic construct
  REGION 1..522: note = an artificially synthesised sequence
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1034 - protein, 984 aa, synthetic construct
  REGION 1..984: note = an artificially synthesised sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSHREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1035 - protein, 984 aa, synthetic construct
  REGION 1..984: note = an artificially synthesised sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSHHEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1036 - protein, 984 aa, synthetic construct
  REGION 1..984: note = an artificially synthesised sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSHKEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1037 - protein, 984 aa, synthetic construct
  REGION 1..984: note = an artificially synthesised sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSHSEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1038 - protein, 985 aa, synthetic construct
  REGION 1..985: note = an artificially synthesised sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKHREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1039 - protein, 985 aa, synthetic construct
  REGION 1..985: note = an artificially synthesised sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKQREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1040 - protein, 985 aa, synthetic construct
  REGION 1..985: note = an artificially synthesised sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKEREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1041 - protein, 985 aa, synthetic construct
  REGION 1..985: note = an artificially synthesised sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKPREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1042 - protein, 983 aa, synthetic construct
  REGION 1..983: note = an artificially synthesised sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1043 - protein, 983 aa, synthetic construct
  REGION 1..983: note = an artificially synthesised sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKHHEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1044 - protein, 983 aa, synthetic construct
  REGION 1..983: note = an artificially synthesised sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKHREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1045 - protein, 983 aa, synthetic construct
  REGION 1..983: note = an artificially synthesised sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKKHEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1046 - protein, 983 aa, synthetic construct
  REGION 1..983: note = an artificially synthesised sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKRHEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1047 - protein, 982 aa, synthetic construct
  REGION 1..982: note = an artificially synthesised sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1048 - protein, 982 aa, synthetic construct
  REGION 1..982: note = an artificially synthesised sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKHEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1049 - protein, 982 aa, synthetic construct
  REGION 1..982: note = an artificially synthesised sequence
QVQLQQSGPQLVRPGASVKISCKASGYSFTSYWMHWVNQRPGQGLEWIGMIDPSYSETRLNQKFKDKATLTVDKSSSTAYMQLSSPTSEDSAVYYCALYGNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKKEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1050 - protein, 1007 aa, synthetic construct
  REGION 1..1007: note = an artificially synthesised sequence
QVQLVQSGAEVKKPGSSVKVSCKASGGTFSDYYLHWSRQAPGQGLEWMGWIDPENGDTEYAPKFQGRVTITADESTSTAYMELSSLRSEDTAVYYCARNKELRYIDVWGQGTLVTVSSTSTSGRSANPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1051 - protein, 22 aa, synthetic construct
  REGION 1..22: note = an artificially synthesised sequence
GGGGSTSTSGRSANPRGGGGGS

SEQ ID NO: 1052 - protein, 5 aa, synthetic construct
  REGION 1..5: note = an artificially synthesised sequence
KSHRE

SEQ ID NO: 1053 - protein, 5 aa, synthetic construct
  REGION 1..5: note = an artificially synthesised sequence
KSHHE

SEQ ID NO: 1054 - protein, 5 aa, synthetic construct
  REGION 1..5: note = an artificially synthesised sequence
KSHKE

SEQ ID NO: 1055 - protein, 5 aa, synthetic construct
  REGION 1..5: note = an artificially synthesised sequence
KSHSE

SEQ ID NO: 1056 - protein, 6 aa, synthetic construct
  REGION 1..6: note = an artificially synthesised sequence
KSKHRE

SEQ ID NO: 1057 - protein, 6 aa, synthetic construct
  REGION 1..6: note = an artificially synthesised sequence
KSKQRE

SEQ ID NO: 1058 - protein, 6 aa, synthetic construct
  REGION 1..6: note = an artificially synthesised sequence
KSKERE

SEQ ID NO: 1059 - protein, 6 aa, synthetic construct
  REGION 1..6: note = an artificially synthesised sequence
KSKPRE

SEQ ID NO: 1060 - protein, 4 aa, synthetic construct
  REGION 1..4: note = an artificially synthesised sequence
KHKE

SEQ ID NO: 1061 - protein, 4 aa, synthetic construct
  REGION 1..4: note = an artificially synthesised sequence
KHHE

SEQ ID NO: 1062 - protein, 4 aa, synthetic construct
  REGION 1..4: note = an artificially synthesised sequence
KHRE

SEQ ID NO: 1063 - protein, 4 aa, synthetic construct
  REGION 1..4: note = an artificially synthesised sequence
KKHE

SEQ ID NO: 1064 - protein, 4 aa, synthetic construct
  REGION 1..4: note = an artificially synthesised sequence
KRHE

SEQ ID NO: 1065-1067 - skipped (3 entries)

SEQ ID NO: 1068 - protein, 522 aa, synthetic construct
  REGION 1..522: note = an artificially synthesised sequence
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSHREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1069 - protein, 522 aa, synthetic construct
  REGION 1..522: note = an artificially synthesised sequence
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSHHEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1070 - protein, 522 aa, synthetic construct
  REGION 1..522: note = an artificially synthesised sequence
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSHKEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1071 - protein, 522 aa, synthetic construct
  REGION 1..522: note = an artificially synthesised sequence
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSHSEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1072 - protein, 523 aa, synthetic construct
  REGION 1..523: note = an artificially synthesised sequence
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKHREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1073 - protein, 523 aa, synthetic construct
  REGION 1..523: note = an artificially synthesised sequence
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKQREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1074 - protein, 523 aa, synthetic construct
  REGION 1..523: note = an artificially synthesised sequence
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKEREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1075 - protein, 523 aa, synthetic construct
  REGION 1..523: note = an artificially synthesised sequence
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKSKPREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1076 - protein, 521 aa, synthetic construct
  REGION 1..521: note = an artificially synthesised sequence
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1077 - protein, 521 aa, synthetic construct
  REGION 1..521: note = an artificially synthesised sequence
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKHHEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1078 - protein, 521 aa, synthetic construct
  REGION 1..521: note = an artificially synthesised sequence
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKHREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1079 - protein, 521 aa, synthetic construct
  REGION 1..521: note = an artificially synthesised sequence
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKKHEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1080 - protein, 521 aa, synthetic construct
  REGION 1..521: note = an artificially synthesised sequence
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKRHEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1081 - protein, 520 aa, synthetic construct
  REGION 1..520: note = an artificially synthesised sequence
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKREKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1082 - protein, 520 aa, synthetic construct
  REGION 1..520: note = an artificially synthesised sequence
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKHEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1083 - protein, 520 aa, synthetic construct
  REGION 1..520: note = an artificially synthesised sequence
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKQYSGRFTCWWLTTISTDLTFSVKSSRGSSDPQGVTCGAATLSAERVRGDNKEYEYSVECQEDSACPAAEESLPIEVMVDAVHKLKYEQYTSSFFIRDIIKPDPPKNLQLKPLKNSRQVEVSWEYPDTWSTPHSYFSLTFCVQVQGKKEKKDRVFTDKTSATVICRKNASISVRAQDRYYSSSWSEWASVPCSGGGGSGGGGSGGGGSGGGGSNLPVAAPDPGMFPCLHHSQNLLRAVSNMLQKARQTLEFYPCTSEEIDHEDITKDKTSTVEACLPLELTKQESCLNSRETSFITQGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLQAS

SEQ ID NO: 1084 - protein, 118 aa, synthetic construct
  REGION 1..118: note = an artificially synthesised sequence
QVQLVQSGAEVKKPGSSVKVSCKASGGTFSDYYLHWSRQAPGQGLEWMGWIDPENGDTEYAPKFQGRVTITADESTSTAYMELSSLRSEDTAVYYCARNKELRYIDVWGQGTLVTVSS

SEQ ID NO: 1085 - protein, 107 aa, synthetic construct
  REGION 1..107: note = an artificially synthesised sequence
DIVMTQSPLSLPVTPGEPASISCRASQSISINLHWYQQKPGQAPRQLIAFASQSISGVPSRFSGSGSGTDFTLTISSLQPEDFATYYCQQSNSFPLTFGQGTKVEIK

SEQ ID NO: 1086 - protein, 107 aa, synthetic construct
  REGION 1..107: note = an artificially synthesised sequence
DIVMTQSPLSLPVTPGEPASISCRASQSIVINLHWYQQKPGQAPRQLIAFASQSISGVPSRFSGSGSGTDFTLTISSLQPEDFATYYCQQSNSFPLTFGQGTKVEIK

SEQ ID NO: 1087 - protein, 328 aa, synthetic construct
  REGION 1..328: note = an artificially synthesised sequence
ASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 1088 - protein, 1007 aa, synthetic construct
QVQLVQSGAEVKKPGSSVKVSCKASGGTFSDYYLHWVRQAPGQGLEWMGWIDPENGDTEYAPKFQGRVTITADESTSTAYMELSSLRSEDTAVYYCARNKELRYFDVWGQGTLVTVSSYSATGRSANPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 1089 - protein, 12 aa, synthetic construct
YSATGRSANPRG

SEQ ID NO: 1090 - protein, 521 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKHKEKKDRVFTDKTSATVICRKNASISVRAQDRYYSSSWSEWASVPCSGGGGSGGGGSGGGGSGGGGSNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 1091 - protein, 121 aa, synthetic construct
QVQLVQSGAEVKKPGASVKVSCKASGYTFTNYYMHWVRQAPGQGLEWVGWINPYTGSAFYAQKFRGRVTMTRDTSISTAYMELSRLRSDDTAVYYCAREPEKFDSDDSDVWGRGTLVTVSS

SEQ ID NO: 1092 - protein, 111 aa, synthetic construct
QAVLTQPPSVSGAPGQRVTISCTGSSSNIGAGYGVHWYQQLPGTAPKLLIYGDSNRPSGVPDRFSGSKSGTSASLAITGLQAEDEADYYCQSYDNSLSGYVFGGGTQLTVL

SEQ ID NO: 1093 - protein, 121 aa, synthetic construct; identical to SEQ ID NO: 914

SEQ ID NO: 1094 - protein, 110 aa, synthetic construct
QAALTQPASVSGSPGQSITISCTGASGDVGAYNFVSWYQQHPGKAPKLIIYDVNKRPSGVSNRFSGSKSGNTASLTISGLQAEDEADYYCSSYTSTFSVVFGGGTKLTVL

SEQ ID NO: 1095 - protein, 617 aa, synthetic construct
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIGGGGSGGGGSQVQLVQSGAEVKKPGASVKVSCKASGYTFTNYYMHWVRQAPGQGLEWVGWINPYTGSAFYAQKFRGRVTMTRDTSISTAYMELSRLRSDDTAVYYCAREPEKFDSDDSDVWGRGTLVTVSSTSTSGRSANPRGASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 1096 - protein, 217 aa, synthetic construct
QAVLTQPPSVSGAPGQRVTISCTGSSSNIGAGYGVHWYQQLPGTAPKLLIYGDSNRPSGVPDRFSGSKSGTSASLAITGLQAEDEADYYCQSYDNSLSGYVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 1097 - protein, 449 aa, synthetic construct
EVQLVESGGGVVRPGGSLRLSCAASGFTFDDYGMNWVRQAPGKGLEWVSGVNWNGGTRDYAASVKGRFTISRDNAKNSLYLQMNSLRAEDTALYHCARGWYSGAAWNMGYWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 1098 - protein, 384 aa, synthetic construct
APISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACIGGGGSGGGGSQAALTQPASVSGSPGQSITISCTGASGDVGAYNFVSWYQQHPGKAPKLIIYDVNKRPSGVSNRFSGSKSGNTASLTISGLQAEDEADYYCSSYTSTFSVVFGGGTKLTVLTSTSGRSANPRGGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 1099 - protein, 635 aa, synthetic construct
EVQLVESGGGVVRPGGSLRLSCAASGFTFDDYGMNWVRQAPGKGLEWVSGVNWNGGTRDYAASVKGRFTISRDNAKNSLYLQMNSLRAEDTALYHCARGWYSGAAWNMGYWGRGTLVTVSSTSTSGRSANPRGASTKGPSVFPLAPCSKSTSGGTAALGCLVKDYFPEPVTVSWNSGALTSGVHTFPAVLQSSGLYSLSSVVTVPSSSLGTQTYICNVNHKPSNTKVDKRVGGGGSGGGGSEPKSSDKTHTCPPCPAPELRRGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGSGGSGGSGGSGGSGGSAPISSHCRLDKSNFQQPYITNRTFMLAKEASLADNNTDVRLIGEKLFHGVSMSERCYLMKQVLNFTLEEVLFPQSDRFQPYMQEVVPFLARLSNRLSTCHIEGDDLHIQRNVQKLKDTVKKLGESGEIKAIGELDLLFMSLRNACI

SEQ ID NO: 1100 - protein, 216 aa, synthetic construct; identical to SEQ ID NO: 912

SEQ ID NO: 1101 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 1102 - protein, 6 aa, synthetic construct
KSKREK

(19) United States
(12) Patent Application Publication
(10) Pub. No.: US 2024/0343771 A1
(43) Pub. Date: Oct. 17, 2024

(54) PROTEASE-MEDIATED TARGET SPECIFIC CYTOKINE DELIVERY USING FUSION POLYPEPTIDE

(71) Applicant: CHUGAI SEIYAKU KABUSHIKI KAISHA, Tokyo (JP)

(72) Inventors: Vishnu Priyanka Reddy CHICHILI, Singapore (SG); Takeru NAMBU, Singapore (SG); Tomoyuki IGAWA, Tokyo (JP); Yohei YAMAMOTO, Shizuoka (JP); Motohiko SATO, Shizuoka (JP); Naoka HIRONIWA, Singapore (SG)

(21) Appl. No.: 18/580,385

(22) PCT Filed: Jul. 15, 2022
(86) PCT No.: PCT/JP2022/027893

§ 371 Date: Jan. 18, 2024

(30) Foreign Application Priority Data
Jul. 19, 2021 (JP) 2021-119178

(51) Int. Cl. C07K 14/54; A61K 39/00; C07K 14/435

(52) U.S. Cl. CPC C07K 14/5434; C07K 14/43504; C07K 14/54; A61K 2039/505; C07K 2317/35; C07K 2317/56; C07K 2319/50

(57) ABSTRACT

The present invention relates to fusion proteins comprising a ligand binding domain, a protease cleavage site, and a ligand moiety. The fusion proteins of the invention comprise a ligand binding domain, a ligand moiety, and a protease cleavage site that, when activated by cleavage by a protease, restores biological activity of the ligand. The invention also relates to methods of producing the fusion proteins, their uses and pharmaceutical compositions comprising said fusion proteins. The present invention also relates to a method of reducing the association between heavy chain variable domain (VH) and light chain variable domain (VL) within the ligand binding domain that promotes dissociation of one from the other. The present disclosure provides fusion proteins in which the ligand is fused to the C-terminus of the constant region, or the N-terminus of the ligand binding domain.

### TECHNICAL FIELD

The present invention relates to fusion proteins comprising a ligand binding moiety with a ligand binding domain and a protease cleavage site that, when activated by cleavage by a protease, restores biological activity of the ligand. The invention also relates to methods of producing the fusion proteins, their uses and pharmaceutical compositions comprising said fusion proteins. The present invention also relates to a method of reducing the association between heavy chain variable domain (VH) and light chain variable domain (VL) within the ligand binding domain that promotes dissociation of one from the other.

### BACKGROUND ART

The innate ability of our immune system prides in its potency, specificity, and memory. Motivated by these features, immunotherapies are being developed in diverse areas, including infectious diseases, autoimmunity, allergies, transplant rejection, graft versus host diseases and cancer. Cytokines and chemokines which are small proteins well known in their roles in the body's immune response to inflammation and immune attack are the centre stage of the development of immunotherapy.

However, to date, there remains a common concern for cytokine mediated immunotherapies regarding high systemic toxicity and low to negligible efficacy. Cytokines, when administered are systemically exposed and therefore elicit toxicity by systemic action, hence often, the cytokines can only be administered at very low doses to circumvent such toxicity. An appealing strategy to overcome this include coupling cytokines to antibodies to locally increase the cytokine concentrations at tumour sites. The cytokine delivered to solid cancer by the immunocytokine activates immunity and thereby exerts an antitumor effect. Since cytokines including IL-2, IL-12, and TNF have strong toxicity, it is expected that the localised action of these cytokines on cancer may be strengthened when delivered by antibodies in a localised delivery while alleviating adverse reactions (NPL1-NPL3). However, it has been reported that such immunocytokines diffuse throughout the body and thus, can bind to any cells in the blood or tissues so long as there are specific, high-affinity cytokine receptors present, leading to unwarranted side effects. In a particular instance, it was reported that an IL-2 fused to antibody binding a cancer antigen exhibited the same anti-tumour effect as an IL-2 fused to antibody that does not bind to the cancer antigen, suggesting the IL-2 moiety directed its biodistribution not the antibody component (NPL4).

Other alternative approaches include having cytokines fused to their receptors via a protease cleavable linker. In an environment, such as a cancer environment, where protease expression is high, the linker is cleaved and the cytokine is released from its receptor. Immunocytokines comprised of such formats include TNF-alpha and TNF-receptor connected via a linker cleavable by urokinase-type plasminogen activator (uPA) (NPL5) and IL-2 and IL-2 receptor cleavable by matrix metalloproteinase-2 (MMP-2) (NPL6). However, the cytokines in these molecules are active even while fused to their receptors, and when activated upon protease cleavage, the improvement in activity is limited, i.e. approximately 10 times.

More recently, a variety of fusion polypeptides comprising cytokines that are released upon protease cleavage have been reported including for example, a single-chain fragment variable (scFv) fused to IL-2 and IL-12 cleavable by matrix metalloproteinases (MMPs) (NPL6, NPL7, PTL2, PTL5) and other fusion polypeptides comprising a protease cleavable region as reported in PTL1, PTL3, PTL4, PTL6, PTL7 and PTL8.

### CITATION LIST

### Patent Literature

[PTL 1] WO 2009/025846
[PTL 2] WO 2011/123683
[PTL 3] WO 2018/097307
[PTL 4] WO 2019/107380
[PTL 5] WO 2019/010219
[PTL 6] WO 2019/010224
[PTL 7] WO 2020/061526
[PTL 8] WO 2021/016640

### Non Patent Literature

[NPL 1] Cyclophosphamide and tucotuzumab (huKS-IL2) following first-line chemotherapy in responding patients with extensive-disease small-cell lung cancer. Gladkov O, Ramlau R, Serwatowski P, Milanowski J, Tomeczko J, Komarnitsky P B, Kramer D, Krzakowski M J. Anticancer Drugs. 2015 November; 26 (10): 1061-8.
[NPL 2] Defining the Pharmacodynamic Profile and Therapeutic Index of NHS-IL12 Immunocytokine in Dogs with Malignant Melanoma. Paoloni M, Mazcko C, Selting K, Lana S, Barber L, Phillips J, Skorupski K, Vail D, Wilson H, Biller B, Avery A, Kiupel M, LeBlanc A, Bernhardt A, Brunkhorst B, Tighe R, Khanna C. PLoS One. 2015 Jun. 19; 10 (6): e0129954.
[NPL 3] Isolated limb perfusion with the tumor-targeting human monoclonal antibodycytokine fusion protein L19-TNF plus melphalan and mild hyperthermia in patients with locally advanced extremity melanoma. Papadia F, Basso V, Patuzzo R, MaurichiA, Di Florio A, Zardi L, Ventura E, Gonzalez-Iglesias R, Lovato V, Giovannoni L, Tasciotti A, Neri D, Santinami M, Menssen H D, De Cian F. J Surg Oncol. 2013 February; 107 (2): 173-9.
[NPL 4] Antigen specificity can be irrelevant to immunocytokine efficacy and biodistribution. Tzeng A, Kwan B H, Opel C F, Navaratna T, Wittrup K D. Proc Natl Acad Sci USA. 2015 Mar. 17; 112 (11): 3320-5.
[NPL 5] Cancer Immunol Immunother. 2006 December; 55 (12): 1590-600. Epub 2006 Apr. 25. Target-selective activation of a TNF prodrug by urokinase-type plasminogen activator (uPA) mediated proteolytic processing at the cell surface. Gerspach J I, Nemeth J, Munkel S, Wajant H, Pfizenmaier K.
[NPL 6] Immunology. 2011 June; 133 (2): 206-20. doi:10.1111/j.1365-2567.2011.03428.x. Epub 2011 Mar. 23. Development of an attenuated interleukin-2 fusion protein that can be activated by tumour-expressed proteases.
Puskas J1, Skrombolas D, Sedlacek A, Lord E, Sullivan M, Frelinger J.
[NPL 7] Development of an Interleukin-12 Fusion Protein That Is Activated by Cleavage with Matrix Metalloproteinase 9. Skrombolas D, Sullivan M, Frelinger J G. J Interferon Cytokine Res. 2019 April; 39(4):233-245

### SUMMARY OF INVENTION

### Technical Problem

It is well known that cytokines are key immune mediators residing in many lesion sites whose effects when harnessed can significantly improve immune responses. While many cytokine-mediated immune therapies have been developed, the issue of high toxicity and low efficacy remain of concern.

### Solution to the Problem

The present inventors have thought that the ability to deliver a site-specifically activated cytokine or chemokine at high dose would overcome systemic toxicity and low efficacy issue.

To this end, the present inventors have developed fusion proteins that comprise a ligand-binding moiety comprising a ligand-binding domain and a protease cleavable site where in a first state, the ligand is bound to the ligand-binding domain and its ability to bind a binding partner is attenuated, and in a second state, the ligand is not bound to the ligand-binding domain and its ability to bind to a binding partner is restored and able to exert its biological activity upon binding thereof. In one nonexclusive aspect, the ligand is bound to the C-terminal region of the constant region of the ligand-binding moiety of the fusion protein by a non-cleavable peptide linker and remains bound regardless of protease cleavage and is capable of interacting with its binding partner and exert its biological activity.

In one nonexclusive aspect, the fusion protein comprises an IgG antibody-like molecule and is a bivalent homodimer, ligand-binding fusion protein, comprising two ligand-binding moieties, each comprising a ligand-binding domain with a protease cleavage site and one ligand bound to the ligand-binding domain. Such fusion proteins and pharmaceutical compositions comprising the fusion protein thereof are useful in the treatment of a disease mediated by the ligand. In one nonexclusive aspect, a method of administering the fusion proteins and pharmaceutical compositions comprising the fusion protein thereof for the treatment of a disease mediated by said ligand, or a method of production of the fusion protein are also included. The present inventors have found that an activated form of the fusion protein is capable of accumulating in high concentrations at the disease site and exhibit fast clearance from the site when compared to the natural ligand. This offers advantage of administering the fusion protein in a higher dose with lesser side effects when compared to the natural ligand and other molecular formats described in the prior art that delivers the natural ligand in their activated form.

### EXEMPLARY EMBODIMENTS

The present invention is based on such findings, and specifically includes the exemplary embodiments described below.
[A-1] A bivalent homodimer fusion protein comprising two polypeptides, each
represented by the general formula (I) from the N- to the C-terminus:
   [ligand-binding domain]-[Lx]-[Cx]-[Ly]-[ligand moiety] (I) wherein:
Lx represents a peptide linker comprising a protease cleavage site,
Cx represents a constant region comprising a second peptide linker and optionally one or more amino acid residues which are modified from or to cysteine;
Ly represents a third peptide linker,
and wherein (a) in a first state, the ligand moiety is bound by the ligand-binding domain and the biological activity of the ligand moiety is attenuated, and in a second state, the biological activity of the ligand moiety is restored, and (b) the fusion protein in the first state has a longer half-life in blood than in the second state, and (c) switching from the first state to the second state is mediated by the presence of a protease that catalyses said protease cleavage site.
[A-2] The fusion protein of [A-1], wherein the ligand-binding domain comprises an antibody variable region.
[A-3] The fusion protein of [A-2], wherein the antibody variable region comprises a heavy chain variable domain (VH) and a light chain variable domain (VL).
[A-4] The fusion protein of [A-3], wherein the heavy chain variable domain (VH) and light chain variable domain (VL) of the ligand-binding domain associates with each other.
[A-5] The fusion protein of [A-4], wherein Cx comprises a CH1 region of the heavy chain and a CL region of the light chain.
[A-6] The fusion protein of any of [A-1]-[A-5], wherein the second peptide linker is positioned in the hinge region so that disulphide bond formation between Cys at position 220 (C220) of the heavy chain and Cys at position 214 (C214) of the light chain (according to EU numbering) is promoted.
[A-7] The fusion protein of any of [A-1]-[A-5], wherein Cx comprises at least one amino acid modification wherein amino acid residues in the heavy chain and the light chain are modified so that no disulphide bond is formed between position 220 of the heavy chain and position 214 of the light chain (according to EU numbering).
[A-8] The fusion protein of [A-7], wherein the light chain comprises C214S modification and the heavy chain comprises C220S modification (according to EU numbering).
[A-9] The fusion protein of any of [A-1]-[A-5], wherein the heavy chain is modified to allow disulphide bond formation between position 131 of the heavy chain and position 214 of the light chain (according to EU numbering).
[A-10] The fusion protein of [A-9], wherein the heavy chain comprises S131C and C220S modifications (according to EU numbering).
[A-11] The fusion protein of any of [A-1] to [A-10], wherein Cx comprises a sequence selected from the group consisting of SEQ ID NO: 901 (C1), SEQ ID NO: 905 (C2), SEQ ID NO: 908 (C3), SEQ ID NO: 910 (C4) and SEQ ID NO: 932 (C5).
[A-12] The fusion protein of [A-11], wherein Cx comprises a sequence of SEQ ID NO: 910 (C4).
[A-13] The fusion protein of any of [A-1] to [A-12], wherein Ly comprises a glycine-serine polymer.
[A-14] The fusion protein of [A-13], wherein the glycine-serine polymer is selected from the group consisting of (a) to (ee):

| | |
|---|---|
| (a) | Ser; |
| (b) | Gly Ser (GS); |
| (c) | Ser Gly (SG); |
| (d) | Gly Gly Ser (GGS); |
| (e) | Gly Ser Gly (GSG); |
| (f) | Ser Gly Gly (SGG); |
| (g) | Gly Ser Ser (GSS); |
| (h) | Ser Ser Gly (SSG); |
| (i) | Ser Gly Ser (SGS); |
| (j) | Gly Gly Gly Ser (GGGS, SEQ ID NO: 136); |
| (k) | Gly Gly Ser Gly (GGSG, SEQ ID NO: 137); |
| (l) | Gly Ser Gly Gly (GSGG, SEQ ID NO: 138); |
| (m) | Ser Gly Gly Gly (SGGG, SEQ ID NO: 139); |
| (n) | Gly Ser Ser Gly (GSSG, SEQ ID NO: 140); |
| (o) | Gly Gly Gly Gly Ser (GGGGS, SEQ ID NO: 141); |
| (p) | Gly Gly Gly Ser Gly (GGGSG, SEQ ID NO: 142); |
| (q) | Gly Gly Ser Gly Gly (GGSGG, SEQ ID NO: 143); |
| (r) | Gly Ser Gly Gly Gly (GSGGG, SEQ ID NO: 144); |
| (s) | Gly Ser Gly Gly Ser (GSGGS, SEQ ID NO: 145); |
| (t) | Ser Gly Gly Gly Gly (SGGGG, SEQ ID NO: 146); |
| (u) | Gly Ser Ser Gly Gly (GSSGG, SEQ ID NO: 147); |
| (v) | Gly Ser Gly Ser Gly (GSGSG, SEQ ID NO: 148); |
| (w) | Ser Gly Gly Ser Gly (SGGSG, SEQ ID NO: 149); |
| (x) | Gly Ser Ser Ser Gly (GSSSG, SEQ ID NO: 150); |
| (y) | Gly Gly Gly Gly Gly Ser (GGGGGS, SEQ ID NO: |
| | 151); |
| (z) | Ser Gly Gly Gly Gly Gly (SGGGGG, SEQ ID NO: |
| | 152); |
| (aa) | Gly Gly Gly Gly Gly Gly Ser (GGGGGGS, SEQ ID |
| | NO: 153); |
| (bb) | Ser Gly Gly Gly Gly Gly Gly (SGGGGGG, SEQ ID |
| | NO: 154); |
| (cc) | (Gly Gly Gly Gly Ser (GGGGS, SEQ ID NO: |
| | 141))n; |
| (dd) | (Ser Gly Gly Gly Gly (SGGGG, SEQ ID NO: |
| | 146))n; |
| and | |
| (ee) | (Gly Gly Ser Gly Gly (GGSGG, SEQ ID NO: |
| | 143))n; |

wherein n is an integer of 1 or larger.
[A-15] The fusion protein of [A-14], wherein Ly comprises a sequence of

(SEQ ID NO: 903)
GGSGGSGGSGGSGGSGGS.

[A-16] The fusion protein of any of [A-1] to [A-15], wherein the ligand moiety comprises a cytokine or a chemokine.
[A-17] The fusion protein of [A-16], wherein the ligand moiety is selected from the group consisting of CXCL9, CXCL10, CXCL11, IL-2, IL-7, IL-12, IL-15, IL-18, IL-21, IL-22, IFN-alpha, IFN-beta, IFN-gamma, MIG, I-TAC, RANTES, MIP-1a, MIP-1b, IL-1R1, IL-1R2, IL-1RAcP and IL-1Ra.
[A-18] The fusion protein of [A-17], wherein the ligand moiety is IL-12 or IL-22.
[A-19] The fusion protein of [A-18], wherein the IL-12 comprises at least one amino acid modification that prevents proteolytic degradation when exposed to protease.
[A-20] The fusion protein of [A-19], wherein the IL-12 does not comprise the amino acid sequence of KSKREK (SEQ ID NO: 1102).
[A-21] The fusion protein of [A-19] or [A-20], wherein the at least one amino acid modification is performed at the interface between IL-12 and the ligand-binding domain.
[A-22] The fusion protein of [A-21], wherein after performing the at least one amino acid modification, the IL-12 comprises a modified sequence selected from the group consisting of (a) to (p):

(SEQ ID NO: 1052)
(a) KSHRE;
(SEQ ID NO: 1053)
(b) KSHHE;
(SEQ ID NO: 1054)
(c) KSHKE;
(SEQ ID NO: 1055)
(d) KSHSE;
(SEQ ID NO: 1056)
(e) KSKHRE;
(SEQ ID NO: 1057)
(f) KSKQRE;
(SEQ ID NO: 1058)
(g) KSKERE;
(SEQ ID NO: 1059)
(h) KSKPRE;
(SEQ ID NO: 1060)
(i) KHKE;
(SEQ ID NO: 1061)
(j) KHHE;
(SEQ ID NO: 1062)
(k) KHRE;
(SEQ ID NO: 1063)
(l) KKHE;
(SEQ ID NO: 1064)
(m) KRHE;
(SEQ ID NO: 1065)
(n) KRE;
(SEQ ID NO: 1066)
(o) KHE;
and
(SEQ ID NO: 1067)
(p) KKE.

[A-23] The fusion protein of any of [A-19] to [A-22], wherein the IL-12 comprises the sequence selected from the group consisting of (i) to (xvi):
   (i) an amino acid sequence that is at least 70%, 80%, or 90% identical to SEQ ID NO: 1068;
   (ii) an amino acid sequence that is at least 70%, 80%, or 90% identical to SEQ ID NO: 1069;
   (iii) an amino acid sequence that is at least 70%, 80%, or 90% identical to SEQ ID NO: 1070;
   (iv) an amino acid sequence that is at least 70%, 80%, or 90% identical to SEQ ID NO: 1071;
   (v) an amino acid sequence that is at least 70%, 80%, or 90% identical to SEQ ID NO: 1072;
   (vi) an amino acid sequence that is at least 70%, 80%, or 90% identical to SEQ ID NO: 1073;
   (vii) an amino acid sequence that is at least 70%, 80%, or 90% identical to SEQ ID NO: 1074;
   (viii) an amino acid sequence that is at least 70%, 80%, or 90% identical to SEQ ID NO: 1075;
   (ix) an amino acid sequence that is at least 70%, 80%, or 90% identical to SEQ ID NO: 1076;
   (x) an amino acid sequence that is at least 70%, 80%, or 90% identical to SEQ ID NO: 1077;
   (xi) an amino acid sequence that is at least 70%, 80%, or 90% identical to SEQ ID NO: 1078;
   (xii) an amino acid sequence that is at least 70%, 80%, or 90% identical to SEQ ID NO: 1079;
   (xiii) an amino acid sequence that is at least 70%, 80%, or 90% identical to SEQ ID NO: 1080;
   (xiv) an amino acid sequence that is at least 70%, 80%, or 90% identical to SEQ ID NO: 1081;
   (xv) an amino acid sequence that is at least 70%, 80%, or 90% identical to SEQ ID NO: 1082; and
   (xvi) an amino acid sequence that is at least 70%, 80%, or 90% identical to SEQ ID NO: 1083.
[A-24] The fusion protein of [A-23], wherein the IL-12 comprises the sequence selected from the group consisting of (i) to (xvi):
   (i) an amino acid sequence that is identical to SEQ ID NO: 1068;
   (ii) an amino acid sequence that is identical to SEQ ID NO: 1069;
   (iii) an amino acid sequence that is identical to SEQ ID NO: 1070;
   (iv) an amino acid sequence that is identical to SEQ ID NO: 1071;
   (v) an amino acid sequence that is identical to SEQ ID NO: 1072;
   (vi) an amino acid sequence that is identical to SEQ ID NO: 1073;
   (vii) an amino acid sequence that is identical to SEQ ID NO: 1074;
   (viii) an amino acid sequence that is identical to SEQ ID NO: 1075;
   (ix) an amino acid sequence that is identical to SEQ ID NO: 1076;
   (x) an amino acid sequence that is identical to SEQ ID NO: 1077;
   (xi) an amino acid sequence that is identical to SEQ ID NO: 1078;
   (xii) an amino acid sequence that is identical to SEQ ID NO: 1079;
   (xiii) an amino acid sequence that is identical to SEQ ID NO: 1080;
   (xiv) an amino acid sequence that is identical to SEQ ID NO: 1081;
   (xv) an amino acid sequence that is identical to SEQ ID NO: 1082; and
   (xvi) (xvi) an amino acid sequence that is identical to SEQ ID NO: 1083.
[A-25] The fusion protein of [A-24], wherein the IL-12 comprises the sequence selected from SEQ ID NO: 1068, or SEQ ID NO: 1069, or SEQ ID NO: 1076, or SEQ ID NO: 1077, or SEQ ID NO: 1078, or SEQ ID NO: 1079, or SEQ ID NO: 1080.
[A-26] The fusion protein of any of [A-1] to [A-25], wherein the fusion protein comprises two protease cleavage sites, and wherein each protease cleavage site is independently cleavable by a protease specific to a target tissue.
[A-27] The fusion protein of [A-26], wherein the target tissue is a cancer tissue or inflammatory tissue.
[A-28] The fusion protein of any of [A-1] to [A-27], wherein each protease cleavage site is cleavable by the same protease.
[A-29] The fusion protein of [A-28], wherein each protease cleavage site comprises the same protease cleavage sequence.
[A-30] The fusion protein of any of [A-1] to [A-29] wherein each protease cleavage site is independently cleavable by a protease selected from the group consisting of matriptase, urokinase-type plasminogen activator (uPA) and matrix metalloprotease (MMP).
[A-31] The fusion protein of any of [A-1] to [A-30], wherein Lx comprises a protease cleavage site which is located near the boundary between the VH and CH1 region or the VL and CL region.
[A-32] The fusion protein of any of [A-1] to [A-31], wherein the ligand-binding domain comprises at least one amino acid modification that reduces association between VH and VL in the second state than in the first state.
[A-33] The fusion protein of [A-32], wherein the modification is a substitution of an amino acid present at the interface between the VH and the VL, and wherein said amino acid residue for modification resides in the Framework region (FR).
[A-34] The fusion protein of [A-33], wherein the substitutions are selected from positions 37, 45, 91 or 103 on the VH, and/or positions 43, 46, 49 or 87 on the VL (according to Kabat numbering).
[A-34a] The fusion protein of [A-33], wherein the substitutions are selected from positions V37, L45, H91, or Y91 or W103 on the VH, and/or positions A43, L46, Y49 or Y87 on the VL (according to Kabat numbering).
[A-35] The fusion protein of [A-34] or [A-34a], wherein each of the positions is substituted to any of A, D, E, F, G, H, I, L, M, N, P, Q, R, S, T, V, W, or Y.
[A-36] The fusion protein of [A-35], wherein the substitution(s) is/are selected from the positions (according to Kabat numbering), comprising any one or more of the following:
V37S,
L45Q,
Y91M, or H91A,
W103I, W103L or W103M on the VH, and/or
A43Q,
L46Q,
Y49A, or
Y87L on the VL.
[A-37] The fusion protein of any of [A-34] to [A-36], wherein the substitutions further comprise at least one modification in an amino acid present at the interface between the ligand-binding domain and the ligand moiety, wherein said amino acid residue for modification resides in the complementarity determining region (CDR).
[A-38] The fusion protein of [A-37], wherein the ligand moiety is IL-12, the substitutions further comprise at least one modification selected from positions 30 on the VL and/or 100a on the VH (according to Kabat numbering).
[A-39] The fusion protein of [A-38], wherein the modification is a substitution selected from S30V and/or F100aI (according to Kabat numbering).
[A-40] The fusion protein of any of [A-34] to [A-39], wherein the substitutions are selected from the group consisting of any one of the following combinations
(a) to (z) according to Kabat numbering:
(a) L46Q and Y49A on the VL;
(b) H91A on the VH, and L46Q and Y49A on the VL;
(c) Y91M on the VH, and A43Q and Y49A on the VL;
(d) Y91M on the VH, and A43Q, L46Q, and Y49A on the VL;
(e) W103M on the VH, and A43Q and Y49A on the VL;
(f) W103M on the VH, and L46Q and Y49A on the VL;
(g) V37S on the VH, and A43Q and Y49A on the VL;
(h) V37S on the VH, and L46Q and Y49A on the VL;
(i) L45Q on the VH, and A43Q and Y49A on the VL;
(j) L45Q on the VH, and L46Q and Y49A on the VL;
(k) F100aI on the VH, and A43Q and Y49A on the VL;
(l) F100aI on the VH, and A43Q, L46Q, and Y49A on the VL;
(m) W103L on the VH, and S30V, L46Q, and Y49A on the VL;
(n) W103M on the VH, and S30V, L46Q, and Y49A on the VL;
(o) V37S and F100aI on the VH, and S30V, A43Q, and Y49A on the VL;
(p) V37S and F100aI on the VH, and S30V, L46Q, and Y49A on the VL;
(q) W103L on the VH, and L46Q and Y49A on the VL;
(r) W103I on the VH, and L46Q and Y49A on the VL;
(s) W103M on the VH, and Y49A and Y87L on the VL;
(t) W103L on the VH, and Y49A and Y87L on the VL;
(u) W103L on the VH, and S30V, Y49A, and Y87L on the VL;
(v) V37S and F100aI on the VH, and L46Q and Y49A on the VL;
(w) V37S and F100aI on the VH, and Y49A and Y87L on the VL;
(x) V37S and F100aI on the VH, and S30V, Y49A, and Y87L on the VL;
(y) V37S, F100aI and W103M on the VH, and L46Q and Y49A on the VL; and
   (z) V37S, F100aI and W103L on the VH, and L46Q and Y49A on the VL.
[A-41] The fusion protein of [A-40], wherein the substitutions are selected from the group consisting of any one of the following combinations (a) to (g) according to Kabat numbering:
(a) W103M on the VH, and L46Q and Y49A on the VL;
(b) W103L on the VH, and S30V, L46Q, and Y49A on the VL;
(c) V37S and F100aI on the VH, and S30V, L46Q, and Y49A on the VL;
(d) W103L on the VH, and L46Q and Y49A on the VL;
(e) V37S and F100aI on the VH, and L46Q and Y49A on the VL;
(f) V37S, F100aI and W103M on the VH, and L46Q and Y49A on the VL; and
   (g) V37S, F100aI and W103L on the VH, and L46Q and Y49A on the VL.
[A-42] The fusion protein of any of [A-1]-[A-41], wherein the molecular weight of the fusion protein in the second state is smaller than the molecular weight of the fusion protein in the first state.
[A-43] The fusion protein of any of [A-1]-[A-42], wherein the cleavage site is cleaved so that a portion of the ligand-binding domain is released from the fusion protein.
[A-44] The fusion protein of [A-43], wherein the molecular weight of the portion of ligand-binding domain released from the fusion protein is 26 kDa, or 13 kDa, or smaller.
[A-45] The fusion protein of any of [A-42]-[A-44] wherein the ratio of the molecular weight of the fusion protein in the first state and the molecular weight of the fusion protein in the second state is 10:9.
[A-46] The fusion protein of [A-42]-[A-44], wherein the molecular weight of the fusion protein in the second state is 9/10 that of the molecular weight of the fusion protein in the first state.
[A-47] The fusion protein of any of [A-42]-[A-44], wherein the percentage reduction in molecular weight of the fusion protein in the second state compared to the fusion protein in the first state is 10%.
[A-48] The fusion protein of any of [A-43]-[A-47], wherein the portion of ligand-binding domain released from the fusion protein comprises VL or VH, or preferably is VL or VH.
[A-49] The fusion protein of any of [A-32]-[A-48], wherein the reduction in association between VH and VL in the second state than in the first state can be represented by a percentage reduction in maximum response unit (RU) of less than or equivalent to 1%, or is less than or equivalent to 2%, or is less than or equivalent to 3%, or is less than or equivalent to 4%, or is less than or equivalent to 5%, or is less than or equivalent to 6%, or is less than or equivalent to 7% or is less than or equivalent to 8%, or is less than or equivalent to 9%, or is less than or equivalent to 10%, or is less than or equivalent to 11%, or is less than or equivalent to 12%, or is less than or equivalent to 13%, or is less than or equivalent to 14%, or is less than or equivalent to 15%, or is less than or equivalent to 16%, or is less than or equivalent to 17%, or is less than or equivalent to 18%, or is less than or equivalent to 19%, or is less than or equivalent to 20%, as measured under surface plasma resonance (SPR) comparing RU of the fusion protein before and after protease cleavage.
[A-50] The fusion protein of any of [A-32]-[A-49], wherein the reduction in association between VH and VL in the second state than in the first state can be represented by a percentage reduction in maximum response unit (RU) of less than or equivalent to 1%, or is less than or equivalent to 2%, or is less than or equivalent to 3%, or is less than or equivalent to 4%, or is less than or equivalent to 5%, or is less than or equivalent to 6%, or is less than or equivalent to 7%, as measured under surface plasma resonance (SPR) comparing RU of the fusion protein before and after protease cleavage.
[A-51] The fusion protein of any of [A-32]-[A-48], wherein the reduction in association between VH and VL in the second state than in the first state can be represented by a percentage reduction in maximum response unit (RU) of less than or equivalent to 15%, or is less than or equivalent to 16%, or is less than or equivalent to 17%, or is less than or equivalent to 18%, or is less than or equivalent to 19%, or is less than or equivalent to 20%, or is less than or equivalent to 21%, or is less than or equivalent to 22%, or is less than or equivalent to 23%, or is less than or equivalent to 24%, or is less than or equivalent to 25%, or is less than or equivalent to 26%, or is less than or equivalent to 27%, or is less than or equivalent to 28%, or is less than or equivalent to 29% or is less than or equivalent to 30%, or is less than or equivalent to 31%, or is less than or equivalent to 32%, or is less than or equivalent to 33%, or is less than or equivalent to 34%, or is less than or equivalent to 35%, or is less than or equivalent to 36%, or is less than or equivalent to 37%, or is less than or equivalent to 38%, or is less than or equivalent to 39%, or is less than or equivalent to 40%, as measured under surface plasma resonance (SPR) comparing RU of the fusion protein before and after protease cleavage.
[A-52] The fusion protein of any of [A-49]-[A-51], wherein SPR conditions comprise a contact duration of the fusion protein in the first state with 400 nM of uPA for a duration of 30 mins.
[A-53] The fusion protein of any of [A-49] to [A-51], wherein the percentage of VH or VL released is directly proportional with the percentage change in response unit (RU) of the fusion protein as measured under SPR in the second state compared to the first state according to formula (II):

$$VH or VL release \% = \% reduction in R U \times 100 / D , \qquad ( II - 1 )$$

wherein D corresponds to 0.01×percentage of molecular weight of VH or VL compared to the molecular weight of the fusion protein in the first state respectively.
[A-54] The fusion protein of [A-53], wherein the percentage of VH or VL released is directly proportional with the percentage change in response unit (RU) of the fusion protein measured under SPR in the second state compared to the first state according to formula (II-1):

$$VH or VL release \% = \% reduction in R U \times 100 / 10. \qquad ( II - 1 )$$

[A-55] The fusion protein of [A-53], wherein the percentage of VH or VL released is directly proportional with the percentage change in response unit (RU) of the fusion protein measured under SPR in the second state compared to the first state according to formula (II-2):

$$VH or VL release \% = \% reduction in R U \times 100 / 15.8 . \qquad ( II - 2 )$$

[A-56] The fusion protein of any of [A-53] to [A-55], wherein the percentage of VH or VL released is more than or equivalent to 10%, or more than or equivalent to 20%, or more than or equivalent to 30%, or more than or equivalent to 40%, or more than or equivalent to 50%, or more than or equivalent to 60%, or more than or equivalent to 70%, or more than or equivalent to 80%, or more than or equivalent to 90%, or more than or equivalent to 100%.
[A-57] The fusion protein of any of [A-1] to [A-56], wherein the ligand moiety in the first and second state remains bound to the constant region via the third peptide linker.
[B-1] A bivalent homodimer fusion protein comprising two polypeptides, each comprising:
   (i) a ligand-binding moiety comprising a ligand-binding domain and a constant region;
   (ii) a first peptide linker comprising a protease cleavage site and connects the ligand-binding domain to the constant region;
   (iii) said constant region comprising a second peptide linker and optionally one or more amino acid residues which are modified from or to cysteine; and
   (iv) a ligand moiety connected to a C-terminal region of the constant region by a third peptide linker,
      wherein (a) in a first state, the ligand moiety is bound by the ligand-binding domain and the biological activity of the ligand moiety is attenuated, and in a second state, the biological activity of the ligand moiety is restored, and (b) the fusion protein in the first state has a longer half-life in blood than in the second state, and (c) switching from the first state to the second state is mediated by the presence of a protease that catalyses said protease cleavage site.
[B-2] The fusion protein of [B-1], wherein the ligand-binding domain comprises an antibody variable region.
[B-3] The fusion protein of [B-2], wherein the antibody variable region comprises a heavy chain variable domain (VH) and a light chain variable domain (VL).
[B-4] The fusion protein of [B-3], wherein the heavy chain variable domain (VH) and light chain variable domain (VL) of the ligand-binding domain associates with each other.
[B-5] The fusion protein of [B-4], wherein the constant region of said ligand-binding moiety comprises a heavy chain and a light chain, wherein the heavy chain comprises a CH1 region and the light chain comprises a CL region.
[B-6] The fusion protein of any of [B-1]-[B-5], wherein the second peptide linker is positioned in the hinge region so that disulphide bond formation between Cys at position 220 (C220) of the heavy chain and Cys at position 214 (C214) of the light chain (according to EU numbering) is promoted.
[B-7] The fusion protein of any of [B-1]-[B-5], wherein the constant region comprises at least one amino acid modification wherein amino acid residues in the heavy chain and the light chain are modified so that no disulphide bond is formed between position 220 of the heavy chain and position 214 of the light chain (according to EU numbering).
[B-8] The fusion protein of [B-7], wherein the light chain comprises C2145 modification and the heavy chain comprises C220S modification (according to EU numbering).
[B-9] The fusion protein of any of [B-1]-[B-5], wherein the heavy chain is modified to allow disulphide bond formation between position 131 of the heavy chain and position 214 of the light chain (according to EU numbering).
[B-10] The fusion protein of [B-10], wherein the heavy chain comprises S131C and C220S modifications (according to EU numbering).
[B-11] The fusion protein of any of [B-1] to [B-10], wherein the constant region comprises a sequence selected from the group consisting of SEQ ID NO: 901 (C1), SEQ ID NO: 905 (C2), SEQ ID NO: 908 (C3), SEQ ID NO: 910 (C4) and SEQ ID NO: 932 (C5).
[B-12] The fusion protein of [B-11], wherein the constant region comprises a sequence of SEQ ID NO: 910 (C4).
[B-13] The fusion protein of any of [B-1] to [B-12], wherein the third peptide linker comprises a glycine-serine polymer.
[B-14] The fusion protein of [B-13], wherein the glycine-serine polymer is selected from the group consisting of (a) to (ee):

| | |
|---|---|
| (a) | Ser; |
| (b) | Gly Ser (GS); |
| (c) | Ser Gly (SG); |
| (d) | Gly Gly Ser (GGS); |
| (e) | Gly Ser Gly (GSG); |
| (f) | Ser Gly Gly (SGG); |
| (g) | Gly Ser Ser (GSS); |
| (h) | Ser Ser Gly (SSG); |
| (i) | Ser Gly Ser (SGS); |
| (j) | Gly Gly Gly Ser (GGGS, SEQ ID NO: 136); |
| (k) | Gly Gly Ser Gly (GGSG, SEQ ID NO: 137); |
| (l) | Gly Ser Gly Gly (GSGG, SEQ ID NO: 138); |
| (m) | Ser Gly Gly Gly (SGGG, SEQ ID NO: 139); |
| (n) | Gly Ser Ser Gly (GSSG, SEQ ID NO: 140); |
| (o) | Gly Gly Gly Gly Ser (GGGGS, SEQ ID NO: 141); |
| (p) | Gly Gly Gly Ser Gly (GGGSG, SEQ ID NO: 142); |
| (q) | Gly Gly Ser Gly Gly (GGSGG, SEQ ID NO: 143); |
| (r) | Gly Ser Gly Gly Gly (GSGGG, SEQ ID NO: 144); |
| (s) | Gly Ser Gly Gly Ser (GSGGS, SEQ ID NO: 145); |
| (t) | Ser Gly Gly Gly Gly (SGGGG, SEQ ID NO: 146); |
| (u) | Gly Ser Ser Gly Gly (GSSGG, SEQ ID NO: 147); |
| (v) | Gly Ser Gly Ser Gly (GSGSG, SEQ ID NO: 148); |
| (w) | Ser Gly Gly Ser Gly (SGGSG, SEQ ID NO: 149); |
| (x) | Gly Ser Ser Ser Gly (GSSSG, SEQ ID NO: 150); |
| (y) | Gly Gly Gly Gly Gly Ser (GGGGGS, SEQ ID NO: |
| | 151); |
| (z) | Ser Gly Gly Gly Gly Gly (SGGGGG, SEQ ID NO: |
| | 152); |
| (aa) | Gly Gly Gly Gly Gly Gly Ser (GGGGGGS, SEQ ID |
| | NO: 153); |
| (bb) | Ser Gly Gly Gly Gly Gly Gly (SGGGGGG, SEQ ID |
| | NO: 154); |
| (cc) | (Gly Gly Gly Gly Ser (GGGGS, SEQ ID NO: |
| | 141))n; |
| (dd) | (Ser Gly Gly Gly Gly (SGGGG, SEQ ID NO: |
| | 146))n; |
| and | |
| (ee) | (Gly Gly Ser Gly Gly (GGSGG, SEQ ID NO: |
| | 143))n; |

wherein n is an integer of 1 or larger.
[B-15] The fusion protein of [B-14], wherein the third peptide linker comprises a sequence of GGSGGSGGSGGSGGSGGS (SEQ ID NO: 903).
[B-16] The fusion protein of any of [B-1] to [B-15], wherein the ligand moiety comprises a cytokine or a chemokine.
[B-17] The fusion protein of [B-16], wherein the ligand moiety is selected from the group consisting of CXCL9, CXCL10, CXCL11, IL-2, IL-7, IL-12, IL-15, IL-18, IL-21, IL-22, IFN-alpha, IFN-beta, IFN-gamma, MIG, I-TAC, RANTES, MIP-1a, MIP-1b, IL-1R1, IL-1R2, IL-1RAcP and IL-1Ra.
[B-18] The fusion protein of [B-17], wherein the ligand moiety is IL-12 or IL-22.
[B-19] The fusion protein of [B-18], wherein the IL-12 comprises at least one amino acid modification that prevents proteolytic degradation when exposed to protease.
[B-20] The fusion protein of [B-19], wherein the IL-12 does not comprise the amino acid sequence of KSKREK (SEQ ID NO: 1102).
[B-21] The fusion protein of [B-19] or [B-20], wherein the at least one amino acid modification is performed at the interface between IL-12 and the ligand-binding domain.
[B-22] The fusion protein of [B-21], wherein after performing the at least one amino acid modification, the IL-12 comprises a modified sequence selected from the group consisting of (a) to (p):

(SEQ ID NO: 1052)
(a) KSHRE;
(SEQ ID NO: 1053)
(b) KSHHE;
(SEQ ID NO: 1054)
(c) KSHKE;
(SEQ ID NO: 1055)
(d) KSHSE;
(SEQ ID NO: 1056)
(e) KSKHRE;
(SEQ ID NO: 1057)
(f) KSKQRE;
(SEQ ID NO: 1058)
(g) KSKERE;
(SEQ ID NO: 1059)
(h) KSKPRE;
(SEQ ID NO: 1060)
(i) KHKE;
(SEQ ID NO: 1061)
(j) KHHE;
(SEQ ID NO: 1062)
(k) KHRE;
(SEQ ID NO: 1063)
(l) KKHE;
(SEQ ID NO: 1064)
(m) KRHE;
(SEQ ID NO: 1065)
(n) KRE;
(SEQ ID NO: 1066)
(o) KHE;
and
(SEQ ID NO: 1067)
(p) KKE.

[B-23] The fusion protein of any of [B-19] to [B-22], wherein the IL-12 comprises the sequence selected from the group consisting of (i) to (xvi):
   (i) an amino acid sequence that is at least 70%, 80%, or 90% identical to SEQ ID NO: 1068;
   (ii) an amino acid sequence that is at least 70%, 80%, or 90% identical to SEQ ID NO: 1069;
   (iii) an amino acid sequence that is at least 70%, 80%, or 90% identical to SEQ ID NO: 1070;
   (iv) an amino acid sequence that is at least 70%, 80%, or 90% identical to SEQ ID NO: 1071;
   (v) an amino acid sequence that is at least 70%, 80%, or 90% identical to SEQ ID NO: 1072;
   (vi) an amino acid sequence that is at least 70%, 80%, or 90% identical to SEQ ID NO: 1073;
   (vii) an amino acid sequence that is at least 70%, 80%, or 90% identical to SEQ ID NO: 1074;
   (viii) an amino acid sequence that is at least 70%, 80%, or 90% identical to SEQ ID NO: 1075;
   (ix) an amino acid sequence that is at least 70%, 80%, or 90% identical to SEQ ID NO: 1076;
   (x) an amino acid sequence that is at least 70%, 80%, or 90% identical to SEQ ID NO: 1077;
   (xi) an amino acid sequence that is at least 70%, 80%, or 90% identical to SEQ ID NO: 1078;
   (xii) an amino acid sequence that is at least 70%, 80%, or 90% identical to SEQ ID NO: 1079;
   (xiii) an amino acid sequence that is at least 70%, 80%, or 90% identical to SEQ ID NO: 1080;
   (xiv) an amino acid sequence that is at least 70%, 80%, or 90% identical to SEQ ID NO: 1081;
   (xv) an amino acid sequence that is at least 70%, 80%, or 90% identical to SEQ ID NO: 1082; and
   (xvi) an amino acid sequence that is at least 70%, 80%, or 90% identical to SEQ ID NO: 1083.
[B-24] The fusion protein of [B-23], wherein the IL-12 comprises the sequence selected from the group consisting of (i) to (xvi):
   (i) an amino acid sequence that is identical to SEQ ID NO: 1068;
   (ii) an amino acid sequence that is identical to SEQ ID NO: 1069;
   (iii) an amino acid sequence that is identical to SEQ ID NO: 1070;
   (iv) an amino acid sequence that is identical to SEQ ID NO: 1071;
   (v) an amino acid sequence that is identical to SEQ ID NO: 1072;
   (vi) an amino acid sequence that is identical to SEQ ID NO: 1073;
   (vii) an amino acid sequence that is identical to SEQ ID NO: 1074;
   (viii) an amino acid sequence that is identical to SEQ ID NO: 1075;
   (ix) an amino acid sequence that is identical to SEQ ID NO: 1076;
   (x) an amino acid sequence that is identical to SEQ ID NO: 1077;
   (xi) an amino acid sequence that is identical to SEQ ID NO: 1078;
   (xii) an amino acid sequence that is identical to SEQ ID NO: 1079;
   (xiii) an amino acid sequence that is identical to SEQ ID NO: 1080;
   (xiv) an amino acid sequence that is identical to SEQ ID NO: 1081;
   (xv) an amino acid sequence that is identical to SEQ ID NO: 1082; and
   (xvi) an amino acid sequence that is identical to SEQ ID NO: 1083.
[B-25] The fusion protein of [B-24], wherein the IL-12 comprises the sequence selected from SEQ ID NO: 1068, or SEQ ID NO: 1069, or SEQ ID NO: 1076, or SEQ ID NO: 1077, or SEQ ID NO: 1078, or SEQ ID NO: 1079, or SEQ ID NO: 1080.
[B-26] The fusion protein of any of [B-1] to [B-25], wherein the fusion protein comprises two protease cleavage sites, and wherein each protease cleavage site is independently cleavable by a protease specific to a target tissue.
[B-27] The fusion protein of [B-26] wherein the target tissue is a cancer tissue or inflammatory tissue.
[B-28] The fusion protein of any of [B-1] to [B-27], wherein each protease cleavage site is cleavable by the same protease.
[B-29] The fusion protein of [B-28], wherein each protease cleavage site comprises the same protease cleavage sequence.
[B-30] The fusion protein of any of [B-1] to [B-29] wherein each protease cleavage site is independently cleavable by a protease selected from the group consisting of matriptase, urokinase-type plasminogen activator (uPA) and matrix metalloprotease (MMP).
[B-31] The fusion protein of any of [B-1] to [B-30], wherein the first peptide linker comprises a protease cleavage site which is located near the boundary between the VH and CH1 region or the VL and CL region.
[B-32] The fusion protein of any of [B-1] to [B-31], wherein the ligand-binding domain comprises at least one amino acid modification that reduces association between VH and VL in the second state than in the first state.
[B-33] The fusion protein of [B-32], wherein the modification is a substitution of an amino acid present at the interface between the VH and the VL, and wherein said amino acid residue for modification resides in the Framework region (FR).
[B-34] The fusion protein of [B-33], wherein the substitutions are selected from position 37, 45, 91 or 103 on the VH, and/or positions 43, 46, 49 or 87 on the VL (according to Kabat numbering).
[B-34a] The fusion protein of [B-33], wherein the substitutions are selected from positions V37, L45, H91, or Y91 or W103 on the VH, and/or positions A43, L46, Y49 or Y87 on the VL (according to Kabat numbering).
[B-35] The fusion protein of [B-34] or [B-34a], wherein each of the positions is substituted to any of A, D, E, F, G, H, I, L, M, N, P, Q, R, S, T, V, W, or Y.
[B-36] The fusion protein of [B-35], wherein the substitution(s) is/are selected from the positions (according to Kabat numbering), comprising any one or more of the following:
V37S,
L45Q,
Y91M, or H91A,
W103I, W103L or W103M on the VH, and/or
A43Q,
L46Q,
Y49A or
Y87L on the VL.
[B-37] The fusion protein of any of [B-34] to [B-36], wherein the substitutions further comprise at least one modification in an amino acid present at the interface between the ligand-binding domain and the ligand moiety, wherein said amino acid residue for modification resides in the complementarity determining region (CDR).
[B-38] The fusion protein of [B-37], wherein the ligand moiety is IL-12, the substitutions further comprise at least one modification selected from positions 30 on the VL and/or 100a on the VH (according to Kabat numbering).
[B-39] The fusion protein of [B-38], wherein the modification is a substitution selected from S30V and/or F100aI (according to Kabat numbering).
[B-40] The fusion protein of [B-34] to [B-39], wherein the substitutions are selected from the group consisting of any one of the following combinations (a) to (z) according to Kabat numbering:
(a) L46Q and Y49A on the VL;
(b) H91A on the VH, and L46Q and Y49A on the VL;
(c) Y91M on the VH, and A43Q and Y49A on the VL;
(d) Y91M on the VH, and A43Q, L46Q, and Y49A on the VL;
(e) W103M on the VH, and A43Q and Y49A on the VL;
(f) W103M on the VH, and L46Q and Y49A on the VL;
(g) V37S on the VH, and A43Q and Y49A on the VL;
(h) V37S on the VH, and L46Q and Y49A on the VL;
(i) L45Q on the VH, and A43Q and Y49A on the VL;
(j) L45Q on the VH, and L46Q and Y49A on the VL;
(k) F100aI on the VH, and A43Q and Y49A on the VL;
(l) F100aI on the VH, and A43Q, L46Q, and Y49A on the VL;
(m) W103L on the VH, and S30V, L46Q, and Y49A on the VL;
(n) W103M on the VH, and S30V, L46Q, and Y49A on the VL;
(o) V37S and F100aI on the VH, and S30V, A43Q, and Y49A on the VL;
(p) V37S and F100aI on the VH, and S30V, L46Q, and Y49A on the VL;
(q) W103L on the VH, and L46Q and Y49A on the VL;
(r) W103I on the VH, and L46Q and Y49A on the VL;
(s) W103M on the VH, and Y49A and Y87L on the VL;
(t) W103L on the VH, and Y49A and Y87L on the VL;
(u) W103L on the VH, and S30V, Y49A, and Y87L on the VL;
(v) V37S and F100aI on the VH, and L46Q and Y49A on the VL;
(w) V37S and F100aI on the VH, and Y49A and Y87L on the VL;
(x) V37S and F100aI on the VH, and S30V, Y49A, and Y87L on the VL;
(y) V37S, F100aI and W103M on the VH, and L46Q and Y49A on the VL; and
   (z) V37S, F100aI and W103L on the VH, and L46Q and Y49A on the VL.
[B-41] The fusion protein of [B-40], wherein the substitutions are selected from the following the group consisting of any one of the following combinations (a) to (g) according to Kabat numbering:
(a) W103M on the VH, and L46Q and Y49A on the VL;
(b) W103L on the VH, and S30V, L46Q, and Y49A on the VL;
(c) V37S and F100aI on the VH, and S30V, L46Q, and Y49A on the VL;
(d) W103L on the VH, and L46Q and Y49A on the VL;
(e) V37S and F100aI on the VH, and L46Q and Y49A on the VL;
(f) V37S, F100aI and W103M on the VH, and L46Q and Y49A on the VL; and
   (g) V37S, F100aI and W103L on the VH, and L46Q and Y49A on the VL.
[B-42] The fusion protein of any of [B-1]-[B-41], wherein the molecular weight of the fusion protein in the second state is smaller than the molecular weight of the fusion protein in the first state.
[B-43] The fusion protein of any of [B-1]-[B-42], wherein the cleavage site is cleaved so that a portion of the ligand-binding domain is released from the fusion protein.
[B-44] The fusion protein of [B-43], wherein the molecular weight of the portion of ligand-binding domain released from the fusion protein is 26 kDa, or 13 kDa, or smaller.
[B-45] The fusion protein of any of [B-42]-[B-44] wherein the ratio of the molecular weight of the fusion protein in the first state and the molecular weight of the fusion protein in the second state is 10:9.
[B-46] The fusion protein of any of [B-42]-[B-44], wherein the molecular weight of the fusion protein in the second state is 9/10 that of the molecular weight of the fusion protein in the first state.
[B-47] The fusion protein of any of [B-42]-[B-44], wherein the percentage reduction in molecular weight of the fusion protein in the second state compared to the fusion protein in the first state is 10%.
[B-48] The fusion protein of any of [B-43]-[B-47], wherein the portion of ligand-binding domain released from the fusion protein comprises VL or VH, or preferably is VL or VH.
[B-49] The fusion protein of any of [B-32]-[B-48], wherein the reduction in association between VH and VL in the second state than in the first state can be represented by a percentage reduction in maximum response unit (RU) of less than or equivalent to 1%, or is less than or equivalent to 2%, or is less than or equivalent to 3%, or is less than or equivalent to 4%, or is less than or equivalent to 5%, or is less than or equivalent to 6%, or is less than or equivalent to 7% or is less than or equivalent to 8%, or is less than or equivalent to 9%, or is less than or equivalent to 10%, or is less than or equivalent to 11%, or is less than or equivalent to 12%, or is less than or equivalent to 13%, or is less than or equivalent to 14%, or is less than or equivalent to 15%, or is less than or equivalent to 16%, or is less than or equivalent to 17%, or is less than or equivalent to 18%, or is less than or equivalent to 19%, or is less than or equivalent to 20%, as measured under surface plasma resonance (SPR) comparing RU of the fusion protein before and after protease cleavage.
[B-50] The fusion protein of any of [B-32]-[B-49], wherein the reduction in association between VH and VL in the second state than in the first state can be represented by a percentage reduction in response unit of less than or equivalent to 1%, or is less than or equivalent to 2%, or is less than or equivalent to 3%, or is less than or equivalent to 4%, or is less than or equivalent to 5%, or is less than or equivalent to 6%, or is less than or equivalent to 7%, as measured under surface plasma resonance (SPR) comparing RU of the fusion protein before and after protease cleavage.
[B-51] The fusion protein of any of [B-32]-[B-48], wherein the reduction in association between VH and VL in the second state than in the first state can be represented by a percentage reduction in maximum response unit (RU) of less than or equivalent to 15%, or is less than or equivalent to 16%, or is less than or equivalent to 17%, or is less than or equivalent to 18%, or is less than or equivalent to 19%, or is less than or equivalent to 20%, or is less than or equivalent to 21%, or is less than or equivalent to 22%, or is less than or equivalent to 23%, or is less than or equivalent to 24%, or is less than or equivalent to 25%, or is less than or equivalent to 26%, or is less than or equivalent to 27%, or is less than or equivalent to 28%, or is less than or equivalent to 29% or is less than or equivalent to 30%, or is less than or equivalent to 31%, or is less than or equivalent to 32%, or is less than or equivalent to 33%, or is less than or equivalent to 34%, or is less than or equivalent to 35%, or is less than or equivalent to 36%, or is less than or equivalent to 37%, or is less than or equivalent to 38%, or is less than or equivalent to 39%, or is less than or equivalent to 40%, as measured under surface plasma resonance (SPR) comparing RU of the fusion protein before and after protease cleavage.
[B-52] The fusion protein of any of [B-49] to [B-51], wherein SPR conditions comprise a contact duration of the fusion protein in the first state with 400 nM of uPA for a duration of 30 mins.
[B-53] The fusion protein of any of [B-49] to [B-51], wherein the percentage of VH or VL released is directly proportional with the percentage change in response unit (RU) of the fusion protein measured under SPR in the second state compared to the first state according to formula (II):

$$VH or VL release \% = \% reduction in R U \times 100 / D . \qquad ( II )$$

wherein D corresponds to 0.01×percentage of molecular weight of VH or VL compared to the molecular weight of the fusion protein in the first state respectively.
[B-54] The fusion protein of [B-53], wherein the percentage of VH or VL released is directly proportional with the percentage change in response unit (RU) of the fusion protein measured under SPR in the second state compared to the first state according to formula (II-1):

$$VH or VL release \% = \% reduction in R U \times 100 / 10. \qquad ( II - 1 )$$

[B-55] The fusion protein of [B-53], wherein the percentage of VH or VL released is directly proportional with the percentage change in response unit (RU) of the fusion protein measured under SPR in the second state compared to the first state according to formula (II-2):

$$VH or VL release \% = \% reduction in R U \times 100 / 15.8 . \qquad ( II - 2 )$$

[B-56] The fusion protein of any of [B-53] to [B-55], wherein the percentage of VH or VL released is more than or equivalent to 20%, or more than or equivalent to 30%, or more than or equivalent to 40%, or more than or equivalent to 50%, or more than or equivalent to 60%, or more than or equivalent to 70%, or more than or equivalent to 80%, or more than or equivalent to 90%, or more than or equivalent to 100%.
[B-57] The fusion protein of any of [B-1] to [B-56], wherein the ligand moiety in the first and second state remains bound to the constant region via the third peptide linker.
[C-1] A bivalent homodimer fusion protein comprising an IgG antibody-like polypeptide fused to a ligand moiety, comprising:
(i) a first peptide linker comprising a protease cleavage site between the boundary of (ia) VH and CH1 region or (ib) VL and CL region;
(ii) a second peptide linker introduced in the hinge region connecting the CH1 region to Fc region of the antibody and optionally comprising one or more amino acid residues which are modified from or to cysteine; and
(iii) a third peptide linker connecting the ligand moiety to C-terminus of the Fc region of the antibody,
wherein (a) in a first state, the ligand moiety is bound by the antibody variable region and the biological activity of the ligand moiety is attenuated, and in a second state, the biological activity of the ligand moiety is restored, and (b) the fusion protein in the first state has a longer half-life in blood than in the second state, and (c) switching from the first state to the second state is mediated by the presence of a protease that catalyses said protease cleavage site.
[C-2] The fusion protein of [C-1], wherein the second peptide linker is positioned in the hinge region so that disulphide bond formation between Cys at position 220 (C220) of the heavy chain and Cys at position 214 (C214) of the light chain (according to EU numbering) is promoted.
[C-3] The fusion protein of [C-1], wherein the constant region comprises at least one amino acid modification wherein amino acid residues in the heavy chain and the light chain are modified so that no disulphide bond is formed between position 220 of the heavy chain and position 214 of the light chain (according to EU numbering).
[C-4] The fusion protein of [C-3], wherein the light chain comprises C214S modification and the heavy chain comprises C220S modification (according to EU numbering).
[C-5] The fusion protein of [C-1], wherein the heavy chain is modified to allow disulphide bond formation between position 131 of the heavy chain and position 214 of the light chain (according to EU numbering).
[C-6] The fusion protein of [C-5], wherein the heavy chain comprises S131C and C220S modifications (according to EU numbering).
[C-7] The fusion protein of any of [C-1] to [C6], wherein the constant region comprises a sequence selected from the group consisting of SEQ ID NO: 901 (C1), SEQ ID NO: 905 (C2), SEQ ID NO: 908 (C3), SEQ ID NO: 910 (C4) and SEQ ID NO: 932 (C5).
[C-8] The fusion protein of [C-7], wherein the constant region comprises a sequence of SEQ ID NO: 910 (C4).
[C-9] The fusion protein of any of [C-1] to [C-8], wherein the third peptide linker comprises a glycine-serine polymer.
[C-10] The fusion protein of [C-9], wherein the glycine-serine polymer is selected from the group consisting of (a) to (ee):

| | |
|---|---|
| (a) | Ser; |
| (b) | Gly Ser (GS); |
| (c) | Ser Gly (SG); |
| (d) | Gly Gly Ser (GGS); |
| (e) | Gly Ser Gly (GSG); |
| (f) | Ser Gly Gly (SGG); |
| (g) | Gly Ser Ser (GSS); |
| (h) | Ser Ser Gly (SSG); |
| (i) | Ser Gly Ser (SGS); |
| (j) | Gly Gly Gly Ser (GGGS, SEQ ID NO: 136); |
| (k) | Gly Gly Ser Gly (GGSG, SEQ ID NO: 137); |
| (l) | Gly Ser Gly Gly (GSGG, SEQ ID NO: 138); |
| (m) | Ser Gly Gly Gly (SGGG, SEQ ID NO: 139); |
| (n) | Gly Ser Ser Gly (GSSG, SEQ ID NO: 140); |
| (o) | Gly Gly Gly Gly Ser (GGGGS, SEQ ID NO: 141); |
| (p) | Gly Gly Gly Ser Gly (GGGSG, SEQ ID NO: 142); |
| (q) | Gly Gly Ser Gly Gly (GGSGG, SEQ ID NO: 143); |
| (r) | Gly Ser Gly Gly Gly (GSGGG, SEQ ID NO: 144); |
| (s) | Gly Ser Gly Gly Ser (GSGGS, SEQ ID NO: 145); |
| (t) | Ser Gly Gly Gly Gly (SGGGG, SEQ ID NO: 146); |
| (u) | Gly Ser Ser Gly Gly (GSSGG, SEQ ID NO: 147); |
| (v) | Gly Ser Gly Ser Gly (GSGSG, SEQ ID NO: 148); |
| (w) | Ser Gly Gly Ser Gly (SGGSG, SEQ ID NO: 149); |
| (x) | Gly Ser Ser Ser Gly (GSSSG, SEQ ID NO: 150); |
| (y) | Gly Gly Gly Gly Gly Ser (GGGGGS, SEQ ID NO: |
| | 151); |
| (z) | Ser Gly Gly Gly Gly Gly (SGGGGG, SEQ ID NO: |
| | 152); |
| (aa) | Gly Gly Gly Gly Gly Gly Ser (GGGGGGS, SEQ ID |
| | NO: 153); |
| (bb) | Ser Gly Gly Gly Gly Gly Gly (SGGGGGG, SEQ ID |
| | NO: 154); |
| (cc) | (Gly Gly Gly Gly Ser (GGGGS, SEQ ID NO: |
| | 141))n; |
| (dd) | (Ser Gly Gly Gly Gly (SGGGG, SEQ ID NO: |
| | 146))n; |
| and | |
| (ee) | (Gly Gly Ser Gly Gly (GGSGG, SEQ ID NO: |
| | 143))n; |

wherein n is an integer of 1 or larger.
[C-11] The fusion protein of [C-10], wherein the third peptide linker comprises a sequence of GGSGGSGGSGGSGGSGGS (SEQ ID NO: 903).
[C-12] The fusion protein of any of [C-1] to [C-11], wherein the ligand moiety comprises a cytokine or a chemokine.
[C-13] The fusion proteins of [C-12], where the ligand moiety is selected from the group consisting of CXCL9, CXCL10, CXCL11, IL-2, IL-7, IL-12, IL-15, IL-18, IL-21, IL-22, IFN-alpha, IFN-beta, IFN-gamma, MIG, I-TAC, RANTES, MIP-1a, MIP-1b, IL-1R1, IL-1R2, IL-1RAcP and IL-1Ra.
[C-14] The fusion protein of [C-13], wherein the ligand moiety is IL-12 or IL-22.
[C-15] The fusion protein of [C-14], wherein the IL-12 comprises at least one amino acid modification that prevents proteolytic degradation when exposed to protease.
[C-16] The fusion protein of [C-15], wherein the IL-12 does not comprise the amino acid sequence of KSKREK (SEQ ID NO: 1102).
[C-17] The fusion protein of [C-15] or [C-16], wherein the at least one amino acid modification is performed at the interface between IL-12 and the antibody variable region.
[C-18] The fusion protein of [C-17], wherein after performing the at least one amino acid modification, the IL-12 comprises a modified sequence selected from the group consisting of (a) to (p):

(SEQ ID NO: 1052)
(a) KSHRE;
(SEQ ID NO: 1053)
(b) KSHHE;
(SEQ ID NO: 1054)
(c) KSHKE;
(SEQ ID NO: 1055)
(d) KSHSE;
(SEQ ID NO: 1056)
(e) KSKHRE;
(SEQ ID NO: 1057)
(f) KSKQRE;
(SEQ ID NO: 1058)
(g) KSKERE;
(SEQ ID NO: 1059)
(h) KSKPRE;
(SEQ ID NO: 1060)
(i) KHKE;
(SEQ ID NO: 1061)
(j) KHHE;
(SEQ ID NO: 1062)
(k) KHRE;
(SEQ ID NO: 1063)
(l) KKHE;
(SEQ ID NO: 1064)
(m) KRHE;
(SEQ ID NO: 1065)
(n) KRE;
(SEQ ID NO: 1066)
(o) KHE;
and
(SEQ ID NO: 1067)
(p) KKE.

[C-19] The fusion protein of any of [C-15] to [C-18], wherein the IL-12 comprises any of the following (i) to (xvi):
   (i) an amino acid sequence that is at least 70%, 80%, or 90% identical to SEQ ID NO: 1068;
   (ii) an amino acid sequence that is at least 70%, 80%, or 90% identical to SEQ ID NO: 1069;
   (iii) an amino acid sequence that is at least 70%, 80%, or 90% identical to SEQ ID NO: 1070;
   (iv) an amino acid sequence that is at least 70%, 80%, or 90% identical to SEQ ID NO: 1071;
   (v) an amino acid sequence that is at least 70%, 80%, or 90% identical to SEQ ID NO: 1072;
   (vi) an amino acid sequence that is at least 70%, 80%, or 90% identical to SEQ ID NO: 1073;
   (vii) an amino acid sequence that is at least 70%, 80%, or 90% identical to SEQ ID NO: 1074;
   (viii) an amino acid sequence that is at least 70%, 80%, or 90% identical to SEQ ID NO: 1075;
   (ix) an amino acid sequence that is at least 70%, 80%, or 90% identical to SEQ ID NO: 1076;
   (x) an amino acid sequence that is at least 70%, 80%, or 90% identical to SEQ ID NO: 1077;
   (xi) an amino acid sequence that is at least 70%, 80%, or 90% identical to SEQ ID NO: 1078;
   (xii) an amino acid sequence that is at least 70%, 80%, or 90% identical to SEQ ID NO: 1079;
   (xiii) an amino acid sequence that is at least 70%, 80%, or 90% identical to SEQ ID NO: 1080;
   (xiv) an amino acid sequence that is at least 70%, 80%, or 90% identical to SEQ ID NO: 1081;
   (xv) an amino acid sequence that is at least 70%, 80%, or 90% identical to SEQ ID NO: 1082; and
   (xvi) an amino acid sequence that is at least 70%, 80%, or 90% identical to SEQ ID NO: 1083.
[C-20] The fusion protein of [C-19], wherein the IL-12 comprises the sequence selected from the group consisting of (i) to (xvi):
(i) an amino acid sequence that is identical to SEQ ID NO: 1068;
(ii) an amino acid sequence that is identical to SEQ ID NO: 1069;
(iii) an amino acid sequence that is identical to SEQ ID NO: 1070;
(iv) an amino acid sequence that is identical to SEQ ID NO: 1071;
(v) an amino acid sequence that is identical to SEQ ID NO: 1072;
(vi) an amino acid sequence that is identical to SEQ ID NO: 1073;
(vii) an amino acid sequence that is identical to SEQ ID NO: 1074;
(viii) an amino acid sequence that is identical to SEQ ID NO: 1075;
(ix) an amino acid sequence that is identical to SEQ ID NO: 1076;
(x) an amino acid sequence that is identical to SEQ ID NO: 1077;
(xi) an amino acid sequence that is identical to SEQ ID NO: 1078;
(xii) an amino acid sequence that is identical to SEQ ID NO: 1079;
(xiii) an amino acid sequence that is identical to SEQ ID NO: 1080;
(xiv) an amino acid sequence that is identical to SEQ ID NO: 1081;
(xv) an amino acid sequence that is identical to SEQ ID NO: 1082; and
(xvi) an amino acid sequence that is identical to SEQ ID NO: 1083.
[C-21] The fusion protein of [C-20], wherein the IL-12 comprises the sequence selected from SEQ ID NO: 1068, or SEQ ID NO: 1069, or SEQ ID NO: 1076, or SEQ ID NO: 1077, or SEQ ID NO: 1078, or SEQ ID NO: 1079, or SEQ ID NO: 1080.
[C-22] The fusion protein of any of [C-1] to [C-21], wherein the fusion protein comprises two protease cleavage sites, and wherein each protease cleavage site is independently cleavable by a protease specific to a target tissue.
[C-23] The fusion protein of [C-22] wherein the target tissue is a cancer tissue or inflammatory tissue.
[C-24] The fusion protein of any of [C-1] to [C-23], wherein each protease cleavage site is cleavable by the same protease.
[C-25] The fusion protein of [C-24], wherein each protease cleavage site comprises the same protease cleavage sequence.
[C-26] The fusion protein of any of [C-1] to [C-25] wherein each protease site is independently cleavable by a protease selected from the group consisting of matriptase, urokinase-type plasminogen activator (uPA) and matrix metalloprotease (MMP).
[C-27] The fusion protein of any of [C-1]-[C-26], wherein the antibody variable region comprises at least one amino acid modification that reduces association between VH and VL in the second state than in the first state.
[C-28] The fusion protein of [C-27], wherein the modification is a substitution of an amino acid present at the interface between the VH and the VL, and wherein said amino acid residue for modification resides in the Framework region (FR).
[C-29] The fusion protein of [C-28], wherein the substitutions are selected from positions 37, 45, 91 or 103 on the VH, and/or positions 43, 46, 49 or 87 on the VL (according to Kabat numbering).
[C-29a] The fusion protein of [C-28], wherein the substitutions are selected from positions V37, L45, H91, Y91 or W103 on the VH, and/or positions A43, L46, Y49 or Y87 on the VL (according to Kabat numbering).
[C-30] The fusion protein of [C-29] or [C-29a], wherein each of the positions is substituted to any of A, D, E, F, G, H, I, L, M, N, P, Q, R, S, T, V, W, or Y.
[C-31] The fusion protein of [C-30], wherein the substitution(s) is/are selected from the positions (according to Kabat numbering), comprising any one or more of the following:
V37S,
L45Q,
Y91M, or H91A,
W103I, W103L or W103M on the VH, and/or
A43Q,
L46Q,
Y49A or
Y87L on the VL.
[C-32] The fusion protein of any of [C-29] to [C-31], wherein the substitutions further comprise at least one modification in an amino acid present at the interface between the ligand-binding domain and the ligand moiety, wherein said amino acid residue for modification resides in the complementarity determining region (CDR).
[C-33] The fusion protein of [C-32], wherein the ligand moiety is IL-12, the substitutions further comprise at least one modification selected from positions 30 on the VL and/or 100a on the VH (according to Kabat numbering).
[C-34] The fusion protein of [C-33], wherein the modification is a substitution selected from S30V and/or F100aI (according to Kabat numbering).
[C-35] The fusion protein of [C-29] to [C-34], wherein the substitutions are selected from the group consisting of any one of the following combinations (a) to (z) according to Kabat numbering:
(a) L46Q and Y49A on the VL;
(b) H91A on the VH, and L46Q and Y49A on the VL;
(c) Y91M on the VH, and A43Q and Y49A on the VL;
(d) Y91M on the VH, and A43Q, L46Q, and Y49A on the VL;
(e) W103M on the VH, and A43Q and Y49A on the VL;
(f) W103M on the VH, and L46Q and Y49A on the VL;
(g) V37S on the VH, and A43Q and Y49A on the VL;
(h) V37S on the VH, and L46Q and Y49A on the VL;
(i) L45Q on the VH, and A43Q and Y49A on the VL;
(j) L45Q on the VH, and L46Q and Y49A on the VL;
(k) F100aI on the VH, and A43Q and Y49A on the VL;
(l) F100aI on the VH, and A43Q, L46Q, and Y49A on the VL;
(m) W103L on the VH, and S30V, L46Q, and Y49A on the VL;
(n) W103M on the VH, and S30V, L46Q, and Y49A on the VL;
(o) V37S and F100aI on the VH, and S30V, A43Q, and Y49A on the VL;
(p) V37S and F100aI on the VH, and S30V, L46Q, and Y49A on the VL;
(q) W103L on the VH, and L46Q and Y49A on the VL;
(r) W103I on the VH, and L46Q and Y49A on the VL;
(s) W103M on the VH, and Y49A and Y87L on the VL;
(t) W103L on the VH, and Y49A and Y87L on the VL;
(u) W103L on the VH, and S30V, Y49A, and Y87L on the VL;
(v) V37S and F100aI on the VH, and L46Q and Y49A on the VL;
(w) V37S and F100aI on the VH, and Y49A and Y87L on the VL;
(x) V37S and F100aI on the VH, and S30V, Y49A, and Y87L on the VL;
(y) V37S, F100aI and W103M on the VH, and L46Q and Y49A on the VL; and
   (z) V37S, F100aI and W103L on the VH, and L46Q and Y49A on the VL.
[C-36] The fusion protein of [C-35], wherein the substitutions are selected from the group consisting of any one of the following combinations (a) to (g) according to Kabat numbering:
(a) W103M on the VH, and L46Q and Y49A on the VL;
(b) W103L on the VH, and S30V, L46Q, and Y49A on the VL;
(c) V37S and F100aI on the VH, and S30V, L46Q, and Y49A on the VL;
(d) W103L on the VH, and L46Q and Y49A on the VL;
(e) V37S and F100aI on the VH, and L46Q and Y49A on the VL;
(f) V37S, F100aI and W103M on the VH, and L46Q and Y49A on the VL; and
   (g) V37S, F100aI and W103L on the VH, and L46Q and Y49A on the VL.
[C-37] The fusion protein of any of [C-1] to [C-36], wherein the molecular weight of the fusion protein in the second state is smaller than the molecular weight of the fusion protein in the first state.
[C-38] The fusion protein of any of [C-1]-[C-37], wherein the cleavage site is cleaved so that a portion of the polypeptide is released from the fusion protein.
[C-39] The fusion protein of [C-38], wherein the molecular weight of the portion that is released from the fusion protein is 26 kDa, or 13 kDa, or smaller.
[C-40] The fusion protein of any of [C-37]-[C-39] wherein the ratio of the molecular weight of the fusion protein in the first state and the molecular weight of the fusion protein in the second state is 10:9.
[C-41] The fusion protein of [C-37]-[C-39], wherein the molecular weight of the fusion protein in the second state is 9/10 that of the molecular weight of the fusion protein in the first state.
[C-42] The fusion protein of any of [C-37]-[C-39], wherein the percentage reduction in molecular weight of the fusion protein in the second state compared to the fusion protein in the first state is 10%.
[C-43] The fusion protein of any of [C-38]-[C-42], wherein the portion that is released from the fusion protein comprises VL or VH, or preferably is VL or VH.
[C-44] The fusion protein of any of [C-27]-[C-43], wherein the reduction in association between VH and VL in the second state than in the first state can be represented by a percentage reduction in maximum response unit (RU) of is less than or equivalent to 1%, or is less than or equivalent to 2%, or is less than or equivalent to 3%, or is less than or equivalent to 4%, or is less than or equivalent to 5%, or is less than or equivalent to 6%, or is less than or equivalent to 7% or is less than or equivalent to 8%, or is less than or equivalent to 9%, or is less than or equivalent to 10%, or is less than or equivalent to 11%, or is less than or equivalent to 12%, or is less than or equivalent to 13%, or is less than or equivalent to 14%, or is less than or equivalent to 15%, or is less than or equivalent to 16%, or is less than or equivalent to 17%, or is less than or equivalent to 18%, or is less than or equivalent to 19%, or is less than or equivalent to 20%, as measured under surface plasma resonance (SPR) comparing RU of the fusion protein before and after protease cleavage.
[C-45] The fusion protein of any of [C-27]-[C-44], wherein the reduction in association between VH and VL in the second state than in the first state can be represented by a percentage reduction in response unit of is less than or equivalent to 1%, or is less than or equivalent to 2%, or is less than or equivalent to 3%, or is less than or equivalent to 4%, or is less than or equivalent to 5%, or is less than or equivalent to 6%, or is less than or equivalent to 7%, as measured under surface plasma resonance (SPR) comparing RU of the fusion protein before and after protease cleavage.
[C-46] The fusion protein of any of [C-27]-[C-43], wherein the reduction in association between VH and VL in the second state than in the first state can be represented by a percentage reduction in maximum response unit (RU) is less than or equivalent to 15%, or is less than or equivalent to 16%, or is less than or equivalent to 17%, or is less than or equivalent to 18%, or is less than or equivalent to 19%, or is less than or equivalent to 20%, or is less than or equivalent to 21%, or is less than or equivalent to 22%, or is less than or equivalent to 23%, or is less than or equivalent to 24%, or is less than or equivalent to 25%, or is less than or equivalent to 26%, or is less than or equivalent to 27%, or is less than or equivalent to 28%, or is less than or equivalent to 29% or is less than or equivalent to 30%, or is less than or equivalent to 31%, or is less than or equivalent to 32%, or is less than or equivalent to 33%, or is less than or equivalent to 34%, or is less than or equivalent to 35%, or is less than or equivalent to 36%, or is less than or equivalent to 37%, or is less than or equivalent to 38%, or is less than or equivalent to 39%, or is less than or equivalent to 40%, as measured under surface plasma resonance (SPR) comparing RU of the fusion protein before and after protease cleavage.
[C-47] The fusion protein of any of [C-27]-[C-46], wherein SPR conditions comprise a contact duration of the fusion protein in the first state with 400 nM of uPA for a duration of 30 mins.
[C-48] The fusion protein of any of [C-27] to [A-47], wherein the percentage of VH or VL released is directly proportional with the percentage change in response unit (RU) of the fusion protein measured under SPR in the second state compared to the first state according to formula (II):

$$VH or VL release \% = \% reduction in R U \times 100 / D , \qquad ( II )$$

wherein D corresponds to 0.01×percentage of molecular weight of VH or VL compared to the molecular weight of the fusion protein in the first state respectively.
[C-49] The fusion protein of [C-48], wherein the percentage of VH or VL released is directly proportional with the percentage change in response unit (RU) of the fusion protein measured under SPR in the second state compared to the first state according to formula (II-1):

$$VH or VL release \% = \% reduction in R U \times 100 / 10. \qquad ( II - 1 )$$

[C-50] The fusion protein of [C-48], wherein the percentage of VH or VL released is directly proportional with the percentage change in response unit (RU) of the fusion protein measured under SPR in the second state compared to the first state according to formula (II-2):

$$VH or VL release \% = \% reduction in R U \times 100 / 15.8 . \qquad ( II - 2 )$$

[C-51] The fusion protein of any of [C-48] to [C-50], wherein the percentage of VH or VL released is more than or equivalent to 10%, or more than or equivalent to 20%, or more than or equivalent to 30%, or more than or equivalent to 40%, or more than or equivalent to 50%, or more than or equivalent to 60%, or more than or equivalent to 70%, or more than or equivalent to 80%, or more than or equivalent to 90%, or more than or equivalent to 100%.
[C-52] The fusion protein of any of [C-1] to [C-51], wherein the ligand moiety in the first and second state remains bound to the constant region via the third peptide linker.
[D-1] A bivalent homodimer fusion protein comprising IL-12, comprising any one of the following sequences (i) to (v):
   (i) a heavy chain variable domain (VH) comprising the amino acid sequence that is at least 70%, 80%, or 90% identical to SEQ ID NO: 1084, and a light chain variable domain (VL) comprising the amino acid sequence that is at least 70%, 80%, or 90% identical to SEQ ID NO: 1085;
   (ii) a heavy chain variable domain (VH) comprising the amino acid sequence that is at least 70%, 80%, or 90% identical to SEQ ID NO: 1084, and a light chain variable domain (VL) comprising the amino acid sequence that is at least 70%, 80%, or 90% identical to SEQ ID NO: 1086;
   (iii) a heavy chain variable domain (VH) comprising the amino acid sequence that is identical to SEQ ID NO: 1084, and a light chain variable domain (VL) comprising the amino acid sequence that is identical to SEQ ID NO: 1085;
   (iv) a heavy chain variable domain (VH) comprising the amino acid sequence that identical to SEQ ID NO: 1084, and a light chain variable domain (VL) comprising the amino acid sequence that is identical to SEQ ID NO: 1086; and
   (v) heavy chain variable domains and light chain variable domains that compete with the heavy chain variable domain and light chain variable domain described in (i) or (iv). [D-2] A bivalent homodimer fusion protein comprising IL-12, comprising any one of the following sequences (i) to (x):
      (i) a light chain comprising the amino acid sequence that is at least 70%, 80%, or 90% identical to SEQ ID NO: 1009, and a heavy chain comprising the amino acid sequence that is at least 70%, 80%, or 90% identical to SEQ ID NO: 1012;
      (ii) a light chain comprising the amino acid sequence that is at least 70%, 80%, or 90% identical to SEQ ID NO: 1016, and a heavy chain comprising the amino acid sequence that is at least 70%, 80%, or 90% identical to SEQ ID NO: 1012;
      (iii) a light chain comprising the amino acid sequence that is at least 70%, 80%, or 90% identical to SEQ ID NO: 1017, and a heavy chain comprising the amino acid sequence that is at least 70%, 80%, or 90% identical to SEQ ID NO: 1012;
      (iv) a light chain comprising the amino acid sequence that is at least 70%, 80%, or 90% identical to SEQ ID NO: 1009, and a heavy chain comprising the amino acid sequence that is at least 70%, 80%, or 90% identical to SEQ ID NO: 1050;
      (v) a light chain comprising the amino acid sequence that is at least 70%, 80%, or 90% identical to SEQ ID NO: 1016, and a heavy chain comprising the amino acid sequence that is at least 70%, 80%, or 90% identical to SEQ ID NO: 1050;
      (vi) (vi) a light chain comprising the amino acid sequence that is at least 70%, 80%, or 90% identical to SEQ ID NO: 1017, and a heavy chain comprising the amino acid sequence that is at least 70%, 80%, or 90%
      (vii) identical to SEQ ID NO: 1050;
         (vii) a light chain comprising the amino acid sequence that is at least 70%, 80%, or 90% identical to SEQ ID NO: 1009, and a heavy chain comprising the amino acid sequence that is at least 70%, 80%, or 90% identical to SEQ ID NO: 1088;
      (viii) a light chain comprising the amino acid sequence that is at least 70%, 80%, or 90% identical to SEQ ID NO: 1016, and a heavy chain comprising the amino acid sequence that is at least 70%, 80%, or 90% identical to SEQ ID NO: 1088;
      (ix) a light chain comprising the amino acid sequence that is at least 70%, 80%, or 90% identical to SEQ ID NO: 1017, and a heavy chain comprising the amino acid sequence that is at least 70%, 80%, or 90% identical to SEQ ID NO: 1088 and;
      (x) heavy chains and light chains that compete with the heavy chain and light chain described in any of (i) to (ix).
[D-3] A bivalent homodimer fusion protein comprising IL-12, comprising any one of the following sequences (i) to (x):
   (i) a light chain comprising the amino acid sequence that is identical to SEQ ID NO: 1009, and a heavy chain comprising the amino acid sequence that is identical to SEQ ID NO: 1012;
   (ii) a light chain comprising the amino acid sequence that is identical to SEQ ID NO: 1016, and a heavy chain comprising the amino acid sequence that is identical to SEQ ID NO: 1012;
   (iii) a light chain comprising the amino acid sequence that is identical to SEQ ID NO: 1017, and a heavy chain comprising the amino acid sequence that is identical to SEQ ID NO: 1012;
   (iv) a light chain comprising the amino acid sequence that is identical to SEQ ID NO: 1009, and a heavy chain comprising the amino acid sequence that is identical to SEQ ID NO: 1050;
   (v) a light chain comprising the amino acid sequence that is identical to SEQ ID NO: 1016, and a heavy chain comprising the amino acid sequence that is identical to SEQ ID NO: 1050;
   (vi) a light chain comprising the amino acid sequence that is identical to SEQ ID NO: 1017, and a heavy chain comprising the amino acid sequence that is identical to SEQ ID NO: 1050;
   (vii) a light chain comprising the amino acid sequence that is identical to SEQ ID NO: 1009, and a heavy chain comprising the amino acid sequence that is identical to SEQ ID NO: 1088;
   (viii) a light chain comprising the amino acid sequence that is identical to SEQ ID NO: 1016, and a heavy chain comprising the amino acid sequence that is identical to SEQ ID NO: 1088;
   (ix) a light chain comprising the amino acid sequence that is identical to SEQ ID NO: 1017, and a heavy chain comprising the amino acid sequence that is identical to SEQ ID NO: 1088; and
   (x) heavy chains and light chains that compete with the heavy chain and light chain described in any of (i) to (ix).
[D-4] A bivalent homodimer fusion protein comprising IL-22, comprising any one of the following sequences (i) to (iv):
   (i) a light chain comprising the amino acid sequence that is, at least 70%, 80%, or 90% identical, or is identical to SEQ ID NO: 1095, and a heavy chain comprising the amino acid sequence that is identical to SEQ ID NO: 1096;
   (ii) a light chain comprising the amino acid sequence that is at least 70%, 80%, or 90% identical, or is identical to SEQ ID NO: 1097, and a heavy chain comprising the amino acid sequence that is identical to SEQ ID NO: 1098;
   (iii) a light chain comprising the amino acid sequence that is at least 70%, 80%, or 90% identical, or is identical to SEQ ID NO: 1099, and a heavy chain comprising the amino acid sequence that is identical to SEQ ID NO: 1100; and
   (iv) heavy chains and light chains that compete with the heavy chain and light chain described in any of (i) to (iii).
[D-5] A bivalent homodimer fusion protein comprising IL-22, comprising any one of the following sequences (i)-(iii):
   (i) a heavy chain variable domain (VH) comprising the amino acid sequence that is at least 70%, 80%, or 90% identical, or is identical to SEQ ID NO: 1091, and a light chain variable domain (VL) comprising the amino acid sequence that is at least 70%, 80%, or 90% identical, or is identical to SEQ ID NO: 1092;
   (ii) a heavy chain variable domain (VH) comprising the amino acid sequence that is at least 70%, 80%, or 90% identical, or is identical to SEQ ID NO: 1093, and a light chain variable domain (VL) comprising the amino acid sequence that is at least 70%, 80%, or 90% identical, or is identical to SEQ ID NO: 1094; and
   (iii) heavy chain variable domains and light chain variable domains that compete with the heavy chain variable domain and light chain variable domain described in (i) or (ii).
[E-1] The fusion protein of any of [A-1] to [A-57], [B-1] to [B-57] and [C-1] to [C-52], comprising any one of the following sequences (i) to (ix):
   (i) a heavy chain variable domain (VH) comprising the amino acid sequence that is at least 70%, 80%, or 90% identical to SEQ ID NO: 1084, and a light chain variable domain (VL) comprising the amino acid sequence that is at least 70%, 80%, or 90% identical to SEQ ID NO: 1085;
   (ii) a heavy chain variable domain (VH) comprising the amino acid sequence that is at least 70%, 80%, or 90% identical to SEQ ID NO: 1084, and a light chain variable domain (VL) comprising the amino acid sequence that is at least 70%, 80%, or 90% identical to SEQ ID NO: 1086;
   (iii) a heavy chain variable domain (VH) comprising the amino acid sequence that is identical to SEQ ID NO: 1084, and a light chain variable domain (VL) comprising the amino acid sequence that is identical to SEQ ID NO: 1085;
   (iv) a heavy chain variable domain (VH) comprising the amino acid sequence that identical to SEQ ID NO: 1084, and a light chain variable domain (VL) comprising the amino acid sequence that is identical to SEQ ID NO: 1086;
   (v) a heavy chain variable domain (VH) comprising the amino acid sequence that is at least 70%, 80%, or 90% identical to SEQ ID NO: 1091, and a light chain variable domain (VL) comprising the amino acid sequence that is at least 70%, 80%, or 90% identical to SEQ ID NO: 1092;
   (vi) a heavy chain variable domain (VH) comprising the amino acid sequence that is at least 70%, 80%, or 90% identical to SEQ ID NO: 1093, and a light chain variable domain (VL) comprising the amino acid sequence that is at least 70%, 80%, or 90% identical to SEQ ID NO: 1094;
   (vii) a heavy chain variable domain (VH) comprising the amino acid sequence that is identical to SEQ ID NO: 1091, and a light chain variable domain (VL) comprising the amino acid sequence that is identical to SEQ ID NO: 1092;
   (viii) a heavy chain variable domain (VH) comprising the amino acid sequence that is identical to SEQ ID NO: 1093, and a light chain variable domain (VL) comprising the amino acid sequence that is identical to SEQ ID NO: 1094; and
   (ix) heavy chain variable domains and light chain variable domains that compete with the heavy chain variable domain and light chain variable domain described in any of (i) to (viii).
[E-2] The fusion protein of any of [A-1] to [A-57], [B-1] to [B-57] and [C-1] to [C-52], comprising any one of the following sequences (i) to (xiii):
   (i) a light chain comprising the amino acid sequence that is at least 70%, 80%, or 90% identical to SEQ ID NO: 1009, and a heavy chain comprising the amino acid sequence that is at least 70%, 80%, or 90% identical to SEQ ID NO: 1012;
   (ii) a light chain comprising the amino acid sequence that is at least 70%, 80%, or 90% identical to SEQ ID NO: 1016, and a heavy chain comprising the amino acid sequence that is at least 70%, 80%, or 90% identical to SEQ ID NO: 1012;
   (iii) a light chain comprising the amino acid sequence that is at least 70%, 80%, or 90% identical to SEQ ID NO: 1017, and a heavy chain comprising the amino acid sequence that is at least 70%, 80%, or 90% identical to SEQ ID NO: 1012;
   (iv) a light chain comprising the amino acid sequence that is at least 70%, 80%, or 90% identical to SEQ ID NO: 1009, and a heavy chain comprising the amino acid sequence that is at least 70%, 80%, or 90% identical to SEQ ID NO: 1050;
   (v) a light chain comprising the amino acid sequence that is at least 70%, 80%, or 90% identical to SEQ ID NO: 1016, and a heavy chain comprising the amino acid sequence that is at least 70%, 80%, or 90% identical to SEQ ID NO: 1050;
   (vi) a light chain comprising the amino acid sequence that is at least 70%, 80%, or 90% identical to SEQ ID NO: 1017, and a heavy chain comprising the amino acid sequence that is at least 70%, 80%, or 90% identical to SEQ ID NO: 1050;
   (vii) a light chain comprising the amino acid sequence that is at least 70%, 80%, or 90% identical to SEQ ID NO: 1009, and a heavy chain comprising the amino acid sequence that is at least 70%, 80%, or 90% identical to SEQ ID NO: 1088;
   (viii) a light chain comprising the amino acid sequence that is at least 70%, 80%, or 90% identical to SEQ ID NO: 1016, and a heavy chain comprising the amino acid sequence that is at least 70%, 80%, or 90% identical to SEQ ID NO: 1088;
   (ix) a light chain comprising the amino acid sequence that is at least 70%, 80%, or 90% identical to SEQ ID NO: 1017, and a heavy chain comprising the amino acid sequence that is at least 70%, 80%, or 90% identical to SEQ ID NO: 1088;
   (x) a light chain comprising the amino acid sequence that is at least 70%, 80%, or 90% identical to SEQ ID NO: 1095, and a heavy chain comprising the amino acid sequence that is at least 70%, 80%, or 90% identical to SEQ ID NO: 1096;
   (xi) a light chain comprising the amino acid sequence that is at least 70%, 80%, or 90% identical to SEQ ID NO: 1097, and a heavy chain comprising the amino acid sequence that is at least 70%, 80%, or 90% identical to SEQ ID NO: 1098;
   (xii) a light chain comprising the amino acid sequence that is at least 70%, 80%, or 90% identical to SEQ ID NO: 1099, and a heavy chain comprising the amino acid sequence that is at least 70%, 80%, or 90% identical to SEQ ID NO: 1100; and
   (xiii) heavy chains and light chains that compete with the heavy chain and light chain described in any of (i) to (xii).
[E-3] The fusion protein of any of [A-1] to [A-57], [B-1] to [B-57] and [C-1] to [C-52], comprising any one of the following sequences (i) to (xiii):
   (i) a light chain comprising the amino acid sequence that is identical to SEQ ID NO: 1009, and a heavy chain comprising the amino acid sequence that is identical to SEQ ID NO: 1012;
   (ii) a light chain comprising the amino acid sequence that is identical to SEQ ID NO: 1016, and a heavy chain comprising the amino acid sequence that is identical to SEQ ID NO: 1012;
   (iii) a light chain comprising the amino acid sequence that is identical to SEQ ID NO: 1017, and a heavy chain comprising the amino acid sequence that is identical to SEQ ID NO: 1012;
   (iv) a light chain comprising the amino acid sequence that is identical to SEQ ID NO: 1009, and a heavy chain comprising the amino acid sequence that is identical to SEQ ID NO: 1050;
   (v) a light chain comprising the amino acid sequence that is identical to SEQ ID NO: 1016, and a heavy chain comprising the amino acid sequence that is identical to SEQ ID NO: 1050;
   (vi) a light chain comprising the amino acid sequence that is identical to SEQ ID NO: 1017, and a heavy chain comprising the amino acid sequence that is identical to SEQ ID NO: 1050;
   (vii) a light chain comprising the amino acid sequence that is identical to SEQ ID NO: 1009, and a heavy chain comprising the amino acid sequence that is identical to SEQ ID NO: 1088;
   (viii) a light chain comprising the amino acid sequence that is identical to SEQ ID NO: 1016, and a heavy chain comprising the amino acid sequence that is identical to SEQ ID NO: 1088;
   (ix) a light chain comprising the amino acid sequence that is identical to SEQ ID NO: 1017, and a heavy chain comprising the amino acid sequence that is identical to SEQ ID NO: 1088;
   (x) a light chain comprising the amino acid sequence that is identical to SEQ ID NO: 1095, and a heavy chain comprising the amino acid sequence that is identical to SEQ ID NO: 1096;
   (xi) a light chain comprising the amino acid sequence that is identical to SEQ ID NO: 1097, and a heavy chain comprising the amino acid sequence that is identical to SEQ ID NO: 1098;
   (xii) a light chain comprising the amino acid sequence that is identical to SEQ ID NO: 1099, and a heavy chain comprising the amino acid sequence that is identical to SEQ ID NO: 1100; and
   (xiii) heavy chains and light chains that compete with the heavy chain and light chain described in any of (i) to (xii).
[E-4] A pharmaceutical composition comprising the fusion protein of any of [A-1] to [A-57], [B-1] to [B-57] and [C-1] to [C-52], [D-1] to [D-5] and [E-1] to [E-3], and [J-1] to [J-55] and a pharmaceutically acceptable carrier.
[E-5] The fusion protein of any of [A-1] to [A-57], [B-1] to [B-57] and [C-1] to [C-52], [D-1] to [D-5] and [E-1] to [E-3] and [J-1] to [J-55] or pharmaceutical composition of [E-4] for use as a medicament.
[E-6] The fusion protein of any of [A-1] to [A-57], [B-1] to [B-57] and [C-1] to [C-52], [D-1] to [D-3] and [E-1] to [E-3] and [J-1] to [J-55] or pharmaceutical composition of [E-4] for use in an IL-12 mediated disease or disorder.
[E-7] The fusion protein of any of [A-1] to [A-57], [B-1] to [B-57] and [C-1] to [C-52], [D-4] or [D-5] and [E-1] to [E-3] and [J-1] to [J-47] or pharmaceutical composition of [E-4] for use in an IL-22 mediated disease or disorder.
[E-8] The fusion protein of any of [A-1] to [A-57], [B-1] to [B-57] and [C-1] to [C-52], [D-1] to [D-5] and [E-3] to [E-4] and [J-1] to [J-55] or pharmaceutical composition of [E-4] for use in treating cancer.
[E-9] The fusion protein of any of [A-1] to [A-57], [B-1] to [B-57] and [C-1] to [C-52], [D-1] to [D-5] and [E-1] to [E-3] and [J-1] to [J-55] or pharmaceutical composition of [E-4] for use in treating inflammatory disease or disorder.
[E-10] Use of the fusion protein of any of [A-1] to [A-57], [B-1] to [B-57] and [C-1] to [C-52], [D-1] to [D-3] and [E-1] to [E-3] and [J-1] to [J-55] or pharmaceutical composition of [E-4] in the manufacture of a medicament for the treatment of an IL-12 mediated disease or disorder.
[E-11] Use of the fusion protein of any of [A-1] to [A-57], [B-1] to [B-57] and [C-1] to [C-52], [D-4] or [D-5] and [E-1] to [E-3] and [J-1] to [J-47] or pharmaceutical composition of [E-4] in the manufacture of a medicament for the treatment of an IL-22 mediated disease or disorder.
[E-12] Use of the fusion protein of any of [A-1] to [A-57], [B-1] to [B-57] and [C-1] to [C-52], [D-1] to [D-5] and [E-1] to [E-3] and [J-1] to [J-55] or pharmaceutical composition of [E-4] in the manufacture of a medicament for the treatment of cancer.
[E-13] Use of the fusion protein of any of [A-1] to [A-57], [B-1] to [B-57] and [C-1] to [C-52], [D-1] to [D-5] and [E-1] to [E-3] and [J-1] to [J-55] or pharmaceutical composition of [E-4] in the manufacture of a medicament for the treatment of inflammatory disease or disorders.
[E-14] A method of treating an individual having an IL-12 mediated disease or disorder comprising administering an effective amount of the fusion protein of any of [A-1] to [A-57], [B-1] to [B-57] and [C-1] to [C-52], [D-1] to [D-3] and [E-1] to [E-3] and [J-1] to [J-55] or pharmaceutical composition of [E-4].
[E-15] A method of treating an individual having an IL-22 mediated disease or disorder comprising administering an effective amount of the fusion protein of any of [A-1] to [A-57], [B-1] to [B-57] and [C-1] to [C-52], [D-4] or [D-5] and [E-1] to [E-3] and [J-1] to [J-47] or pharmaceutical composition of [E-4].
[E-16] A method of treating an individual having cancer comprising administering an effective amount of the fusion protein of any of [A-1] to [A-57], [B-1] to [B-57] and [C-1] to [C-52], [D-1] to [D-5] and [E-1] to [E-3] and [J-1] to [J-55] or pharmaceutical composition of [E-4].
[E-17] A method of treating an individual having an inflammatory disease or disorder comprising administering an effective amount of the fusion protein of any [A-1] to [A-57], [B-1] to [B-57] and [C-1] to [C-52], [D-1] to [D-5] and [E-1] to [E-3] and [J-1] to [J-55] or pharmaceutical composition of [E-4].
[E-18] An isolated polynucleotide encoding the fusion protein of any of [A-1] to [A-57], [B-1] to [B-57] and [C-1] to [C-52], [D-1] to [D-5] and [E-1] to [E-3] and [J-1] to [J-55].
[E-19] A vector comprising the polynucleotide of [E-18].
[E-20] A host cell comprising the polynucleotide of [E-18] or the vector of [E-19].
[E-21] A method of producing the fusion protein of any of [A-1] to [A-57], [B-1] to [B-57] and [C-1] to [C-52], [D-1] to [D-5] and [E-1] to [E-3] and [J-1] to [J-55], comprising the step of culturing the host cell of [E-20] so that the fusion protein is produced.
[E-22] The method according to [E-21], comprising a step of introducing a substitution to an amino acid present at the interface between the VH and the VL to reduce the association between VH and VL in the second state compared to the first state, and wherein said amino acid residue for substitution resides in the Framework region (FR).
[E-23] The method according to [E-22], wherein the amino acid positions for substitution are selected from positions 37, 45, 91 or 103 on the VH, and/or positions 43, 46, 49 or 87 on the VL (according to Kabat numbering).
[E-23a] The method according to [E-22], wherein the amino acid positions for substitution are selected from positions V37, L45, H91, Y91 or W103 on the VH, and/or positions A43, L46, Y49 or Y87 on the VL (according to Kabat numbering).
[E-24] The method according to [E-23] or [E-23a], wherein each of the positions are substituted to any of A, D, E, F, G, H, I, L, M, N, P, Q, R, S, T, V, W, or Y.
[E-25] The method according to [E-24], wherein the substitutions(s) is/are selected from any one or more of the following (according to Kabat numbering):
V37S,
L45Q,
Y91M, or H91A,
W103I, W103L or W103M on the VH, and/or
A43Q,
L46Q,
Y49A or
Y87L on the VL.
[E-26] The method according to any of [E-23] to [E-25], wherein the substitutions further comprise at least one substitution in an amino acid present at the interface between the ligand-binding domain and the ligand moiety, wherein said amino acid residue for modification resides in the complementarity determining region (CDR).
[E-27] The method according to [E-26], wherein the ligand moiety is IL-12, the substitutions further comprise at least one substitution selected from positions 30 on the VL and/or 100a on the VH (according to Kabat numbering).
[E-28] The method according to [E-27], wherein the substitution(s) is/are selected from S30V and/or F100aI (according to Kabat numbering).
[E-29] The method according to any of [E-23] to [E-28], wherein the substitutions are selected from the group consisting of any one of the following combinations
(a) to (bb) according to Kabat numbering:
(a) L46Q and Y49A on the VL;
(b) H91A on the VH, and L46Q and Y49A on the VL;
(c) Y91M on the VH, and A43Q and Y49A on the VL;
(d) Y91M on the VH, and A43Q, L46Q, and Y49A on the VL;
(e) W103M on the VH, and A43Q and Y49A on the VL;
(f) W103M on the VH, and L46Q and Y49A on the VL;
(g) W103I on the VH, and L46Q and Y49A on the VL;
(h) W103L on the VH, and L46Q and Y49A on the VL;
(i) V37S on the VH, and A43Q and Y49A on the VL;
(j) V37S on the VH, and L46Q and Y49A on the VL;
(k) L45Q on the VH, and A43Q and Y49A on the VL;
(1) L45Q on the VH, and L46Q and Y49A on the VL;
(m) F100aI on the VH, and A43Q and Y49A on the VL;
(n) F100aI on the VH, and A43Q, L46Q, and Y49A on the VL;
(o) W103L on the VH, and S30V, L46Q, and Y49A on the VL;
(p) W103M on the VH, and S30V, L46Q, and Y49A on the VL;
(q) V37S and F100aI on the VH, and S30V, A43Q, and Y49A on the VL;
(r) V37S and F100aI on the VH, and S30V, L46Q, and Y49A on the VL;
(s) W103L on the VH, and L46Q and Y49A on the VL;
(t) W103I on the VH, and L46Q and Y49A on the VL;
(u) W103M on the VH, and Y49A and Y87L on the VL;
(v) W103L on the VH, and Y49A and Y87L on the VL;
(w) W103L on the VH, and S30V, Y49A, and Y87L on the VL;
(x) V37S and F100aI on the VH, and L46Q and Y49A on the VL;
(y) V37S and F100aI on the VH, and Y49A and Y87L on the VL;
(z) V37S and F100aI on the VH, and S30V, Y49A, and Y87L on the VL; (aa) V37S, F100aI and W103M on the VH, and L46Q and Y49A on the VL; and
(bb) V37S, F100aI and W103L on the VH, and L46Q and Y49A on the VL.
[E-30] The method according to [E-29], wherein the substitutions are selected from the group consisting of any one of the following combinations (a) to (g) according to Kabat numbering:
(a) W103M on the VH, and L46Q and Y49A on the VL;
(b) W103L on the VH, and S30V, L46Q, and Y49A on the VL;
(c) V37S and F100aI on the VH, and S30V, L46Q, and Y49A on the VL;
(d) W103L on the VH, and L46Q and Y49A on the VL;
(e) V37S and F100aI on the VH, and L46Q and Y49A on the VL;
(f) V37S, F100aI and W103M on the VH, and L46Q and Y49A on the VL; and
   (g) V37S, F100aI and W103L on the VH, and L46Q and Y49A on the VL.
[E-31] The method according to [E-30], further comprising recovering the fusion protein from the host cell.
[E-32] The method according to [E-21], comprising the steps of
   (a) introducing at least one amino acid modification or at least one pair of amino acid modification at the interface between VH and VL in said fusion protein, and optionally introducing at least one amino acid mutation at the interface between the ligand and the ligand-binding domain, that promote dissociation of VH or VL from the fusion protein, (b) confirming that step (a) does not disrupt binding of ligand to VH and VL,
   (c) confirming that step (a) reduced association of VH and VL upon protease cleavage at the protease cleavage site, and
   (d) linking VH or VL of step (a) with an IgG heavy chain constant region via a protease cleavage sequence,
   (e) obtaining a polynucleotide encoding said fusion protein of step (e),
   (f) culturing a host cell comprising the polynucleotide of step (e), and
   (g) producing and recovering the fusion protein from the host cell in step (f).
[F-1] A polypeptide comprising at least one antigen-binding domain comprising a protease cleavage site, whereupon cleavage at the protease cleavage site, an antibody domain adjacent to the protease cleavage site dissociates and wherein the dissociation is promoted by at least one amino acid modification performed at the interface between said antibody domain and a corresponding interacting domain.
[F-2] The polypeptide according to [F-1], wherein the polypeptide is an antibody or antibody fragment.
[F-3] The polypeptide according to [F-2], wherein the antibody is an IgG antibody selected from the group consisting of IgG1, IgG2, IgG3, IgG4, IgG-IgG, IgG-Fab, or CrossMab antibody.
[F-4] The polypeptide according to [F-3], wherein the antibody is monovalent or bivalent.
[F-5] The polypeptide according to [F-4], wherein the antibody is monospecific or bispecific.
[F-6] The polypeptide according to [F-5], wherein the antibody fragment comprises an antigen-binding domain.
[F-7] The polypeptide according to [F-6], wherein the antibody fragment is selected from the group consisting of scFv, scFv-Fc, tandem scFv, Fab, tandem Fab, F(ab′)₂, Fab₂, Fab-scFv-Fc, F(ab′)₂-scFv₂, bispecific Fab₂, trispecific Fab₂, bispecific diabody, trispecific diabody, tandem diabody, triabody, tetrabody, minibody, bibody or tribody.
[F-8] The polypeptide according to [F-7], wherein the antigen-binding domain comprises an antibody variable region.
[F-9] The polypeptide according to [F-8], wherein the antibody variable region comprises a heavy chain variable domain (VH) and a light chain variable domain (VL) that associates with each other, and optionally, wherein the VH is associated with a CH1 region and/or the VL is associated with a CL region.
[F-10] The polypeptide according to [F-9], wherein the protease cleavage site is located at the boundary between VH and CH1 region or VL and CL region, or VH and VL.
[F-11] The polypeptide according to [F-10], wherein the at least one amino acid modification is performed at an interface between VH and VL that reduces the association between VH and VL in the cleaved state compared to the uncleaved state.
[F-12] The polypeptide according to [F-11], wherein at least one pair of amino acid modification is performed at an interface between VH and VL that reduces the association between VH and VL in the cleaved state compared to the uncleaved state.
[F-13] The polypeptide according to [F-11], wherein the at least one amino acid modification is a substitution of an amino acid present at the interface between the VH and the VL, and wherein said amino acid residue for substitution resides in the Framework region (FR).
[F-14] The polypeptide according to [F-12], wherein the at least one pair of amino acid modification is a substitution of an amino acid pair present at the interface between the VH and the VL.
[F-15] The polypeptide according to [F-13], wherein the at least one amino acid substitution comprises substitution of the amino acid to attain the same charge as a corresponding interacting amino acid at the interface between VH and VL or neutral charge.
[F-16] The polypeptide according [F-14], wherein the pair of amino acid substitutions comprises substitutions of both amino acids to have the same charge or neutral charge.
[F-17] The polypeptide according to any of [F-11] to [F-16], wherein the substitutions are selected from positions 37, 39, 44, 45, 47, 91, and 103 on the VH, and/or positions 38, 43, 44, 46, 49, 87, and 98 on the VL (according to Kabat numbering).
[F-17a] The polypeptide according to any of [F-11] to [F-16], wherein the substitutions are selected from positions V37, Q39, G44, L45, W47, H91, Y91 and W103 on the VH, and/or positions R38, A43, P44, L46, Y49, Y87, and F98 on the VL (according to Kabat numbering).
[F-18] The method according to [F-17] or [F-17a], wherein each of the positions is substituted to any of A, D, E, F, G, H, I, L, M, N, P, Q, R, S, T, V, W, or Y.
[F-19] The polypeptide according to [F-18], wherein the substitution(s) is/are selected from the positions (according to Kabat numbering), comprising any one or more of the following:
Q39D,
W47A, W47L, or W47M,
Y91A, Y91L, Y91M, or H91A,
W103A, W103I, W103L, or W103M,
V37S, or V37Q,
G44Q,
L45A, or L45Q on the VH, and/or
R38E,
Y49A,
Y87A, Y87L, or Y87M,
F98A, F98L, or F98M,
A43Q,
P44A, P44S, or P44Q,
L46E, or L46Q on the VL.
[F-20] The polypeptide according to [F-19], wherein the substitutions are selected from the group consisting of any one of the following combinations (a) to (pp) according to Kabat numbering:
(a) L46Q and Y49A on the VL;
(b) Q39D on the VH, and R38E on the VL;
(c) H91A on the VH, and L46Q and Y49A on the VL;
(d) Y91A on the VH, and A43Q and Y49A on the VL;
(e) Y91A on the VH, and P44A and Y49A on the VL;
(f) Y91A on the VH, and L46Q and Y49A on the VL;
(g) Y91A on the VH, and Y49A and Y87L on the VL;
(h) Y91M on the VH, and A43Q and Y49A on the VL;
(i) Y91M on the VH, and P44A and Y49A on the VL;
(j) Y91M on the VH, and L46Q and Y49A on the VL;
(k) Y91M on the VH, and Y49A and Y87L on the VL;
(l) Y91M on the VH, and Y49A and F98L on the VL;
(m) W103L on the VH, and A43Q and Y49A on the VL;
(n) W103L on the VH, and P44A and Y49A on the VL;
(o) W103L on the VH, and L46Q and Y49A on the VL;
(p) W103L on the VH, and Y49A and Y87L on the VL;
(q) W103I on the VH, and A43Q and Y49A on the VL;
(r) W103I on the VH, and P44A and Y49A on the VL;
(s) W103I on the VH, and L46Q and Y49A on the VL;
(t) W103M on the VH, and A43Q and Y49A on the VL;
(u) W103M on the VH, and P44A and Y49A on the VL;
(v) W103M on the VH, and L46Q and Y49A on the VL;
(w) W103M on the VH, and Y49A and Y87L on the VL;
(x) V37S on the VH, and A43Q and Y49A on the VL;
(y) V37S on the VH, and P44A and Y49A on the VL;
(z) V37S on the VH, and L46Q and Y49A on the VL;
(aa) V37S on the VH, and Y49A and Y87L on the VL;
(bb) V37S on the VH, and Y49A and F98L on the VL;
(cc) L45Q on the VH, and A43Q and Y49A on the VL;
(dd) L45Q on the VH, and P44A and Y49A on the VL;
(ee) L45Q on the VH, and L46Q and Y49A on the VL;
(ff) L45Q on the VH, and Y49A and Y87L on the VL;
(gg) L45Q on the VH, and Y49A and F98M on the VL;
(hh) Y91M on the VH, and A43Q, P44A, and Y49A on the VL;
(ii) Y91M on the VH, and A43Q, L46Q, and Y49A on the VL;
(jj) Y91M on the VH, and L46Q, Y49A, and Y87M on the VL;
(kk) V37S on the VH, and L46Q, Y49A, and Y87M on the VL;
(ll) V37S and L45Q on the VH, and A43Q and Y49A on the VL;
(mm) V37S and Y91M on the VH, and A43Q and Y49A on the VL;
(nn) V37S and W103M on the VH, and A43Q and Y49A on the VL;
(oo) V37S and Y91M on the VH, and L46Q and Y49A on the VL; and
(pp) V37S and L45Q on the VH, and Y49A and Y87M on the VL.
[F-21] A pharmaceutical composition comprising the polypeptide of any of [F-1] to [F-20] and a pharmaceutically acceptable carrier.
[F-22] A pharmaceutical composition according to [F-21] or a polypeptide according to any of [F-1] to [F-20] for use as a medicament.
[F-23] A pharmaceutical composition according to [F-21] or a polypeptide according to any of [F-1] to [F-20] for use in a disease or disorder.
[F-24] Use of a pharmaceutical composition according to [F-21] or a polypeptide according to any of [F-1] to [F-20] in the manufacture of a medicament for the treatment of a disease or disorder.
[F-25] A method of treating an individual having a disease or disorder comprising administering an effective amount of the pharmaceutical composition according to [F-21] or a polypeptide according to any of [F-1] to [F-20].
[F-26] An isolated polynucleotide encoding the polypeptide according to any of [F-1] to [F-20].
[F-27] A vector comprising the polynucleotide of [F-26].
[F-28] A host cell comprising the polynucleotide of [F-26] or the vector of [F-27].
[F-29] A method of producing the polypeptide according to any of [F-1] to [F-20], comprising the step of culturing the host cell of [F-28] so that the polypeptide is produced.
[F-30] The method according to [F-29], comprising the steps of
   (a) introducing a peptide linker comprising a protease cleavage site, wherein said protease cleavable peptide linker connects VH to CH1 region, or VL to CL region, or VH to VL,
   (b) introducing at least one substitution mutation into at least one amino acid present at the interface between the VH and the VL to promote dissociation of VH from the VL, or VL from the VH,
   (c) confirming that step (b) does not disrupt binding of antigen to VH and VL, and
   (d) confirming that step (b) reduces association of VH and VL upon protease cleavage at the protease cleavage site,
   (e) obtaining a polynucleotide encoding said polypeptide of step (d),
   (f) culturing a host cell comprising the polynucleotide of step (e), and
   (g) producing and recovering the fusion protein from the host cell in step (f).
[F-31] The method according to [F-30], wherein the substitutions are selected from positions 37, 39, 44, 45, 47, 91, and 103 on the VH, and/or positions 38, 43, 44, 46, 49, 87, and 98 on the VL (according to Kabat numbering).
[F-31a] The method according to [F-30], wherein the substitutions are selected from positions V37, Q39, G44, L45, W47, H91, Y91 and W103 on the VH, and/or positions R38, A43, P44, L46, Y49, Y87, and F98 on the VL (according to Kabat numbering).
[F-32] The method according to [F-31] or [F-31a], wherein each of the positions is substituted to any of A, D, E, F, G, H, I, L, M, N, P, Q, R, S, T, V, W, or Y.
[F-33] The method according to [F-32], wherein the substitution(s) is/are selected from the positions (according to Kabat numbering), comprising any one or more of the following:
   Q39D,
   W47A, W47L, or W47M,
   Y91A, Y91L, Y91M, or H91A,
   W103A, W103I, W103L, or W103M,
   V37S, or V37Q,
   G44Q,
   L45A, or L45Q on the VH, and/or
   R38E,
   Y49A,
   Y87A, Y87L, or Y87M,
   F98A, F98L, or F98M,
   A43Q,
   P44A, P44S, or P44Q,
   L46E, or L46Q on the VL.
[F-34] The method according to [F-33], wherein the substitutions are selected from the group consisting of any one of the following combinations (a) to (pp) according to Kabat numbering:
(a) L46Q and Y49A on the VL;
(b) Q39D on the VH, and R38E on the VL;
(c) H91A on the VH, and L46Q and Y49A on the VL;
(d) Y91A on the VH, and A43Q and Y49A on the VL;
(e) Y91A on the VH, and P44A and Y49A on the VL;
(f) Y91A on the VH, and L46Q and Y49A on the VL;
(g) Y91A on the VH, and Y49A and Y87L on the VL;
(h) Y91M on the VH, and A43Q and Y49A on the VL;
(i) Y91M on the VH, and P44A and Y49A on the VL;
(j) Y91M on the VH, and L46Q and Y49A on the VL;
(k) Y91M on the VH, and Y49A and Y87L on the VL;
(l) Y91M on the VH, and Y49A and F98L on the VL;
(m) W103L on the VH, and A43Q and Y49A on the VL;
(n) W103L on the VH, and P44A and Y49A on the VL;
(o) W103L on the VH, and L46Q and Y49A on the VL;
(p) W103L on the VH, and Y49A and Y87L on the VL;
(q) W103I on the VH, and A43Q and Y49A on the VL;
(r) W103I on the VH, and P44A and Y49A on the VL;
(s) W103I on the VH, and L46Q and Y49A on the VL;
(t) W103M on the VH, and A43Q and Y49A on the VL;
(u) W103M on the VH, and P44A and Y49A on the VL;
(v) W103M on the VH, and L46Q and Y49A on the VL;
(w) W103M on the VH, and Y49A and Y87L on the VL;
(x) V37S on the VH, and A43Q and Y49A on the VL;
(y) V37S on the VH, and P44A and Y49A on the VL;
(z) V37S on the VH, and L46Q and Y49A on the VL;
(aa) V37S on the VH, and Y49A and Y87L on the VL;
(bb) V37S on the VH, and Y49A and F98L on the VL;
(cc) L45Q on the VH, and A43Q and Y49A on the VL;
(dd) L45Q on the VH, and P44A and Y49A on the VL;
(ee) L45Q on the VH, and L46Q and Y49A on the VL;
(ff) L45Q on the VH, and Y49A and Y87L on the VL;
(gg) L45Q on the VH, and Y49A and F98M on the VL;
(hh) Y91M on the VH, and A43Q, P44A, and Y49A on the VL;
(ii) Y91M on the VH, and A43Q, L46Q, and Y49A on the VL;
(jj) Y91M on the VH, and L46Q, Y49A, and Y87M on the VL;
(kk) V37S on the VH, and L46Q, Y49A, and Y87M on the VL;
(ll) V37S and L45Q on the VH, and A43Q and Y49A on the VL;
(mm) V37S and Y91M on the VH, and A43Q and Y49A on the VL;
(nn) V37S and W103M on the VH, and A43Q and Y49A on the VL;
(oo) V37S and Y91M on the VH, and L46Q and Y49A on the VL; and
(pp) V37S and L45Q on the VH, and Y49A and Y87M on the VL.
[F-35] The method according to any of [F-28] to [F-34], further comprising recovering the polypeptide from the host cell.
[G-1] A bivalent homodimer fusion protein comprising a full-length IgG antibody comprising an antigen-binding domain, wherein the antigen-binding domain comprises a variable region, wherein the variable region comprises a heavy chain variable domain (VH) and a light chain variable domain (VL) that associates with each other, and comprises (a) a protease cleavage site at the boundary between VH and CH1 region, or VL and CL region, of its variable region, and (b) a ligand binding to said variable region, and wherein upon protease cleavage, (i) either the VH or the VL dissociates from the fusion protein, and (ii) the ligand dissociates from the variable region, and wherein the dissociation described in (i) is promoted by at least one amino acid modification performed at the interface between VH and VL that reduces association between VH and VL in the cleaved state compared to the uncleaved state.
[G-2] The fusion protein of [G-1], wherein the full-length IgG antibody is an IgG antibody-like polypeptide.
[G-3] The fusion protein of [G-2], wherein the modification is a substitution of an amino acid present at the interface between the VH and the VL.
[G-4] The fusion protein of [G-3], wherein at least one pair of amino acid substitution is performed at the interface between VH and VL, and wherein said amino acid residue for substitution resides in the Framework region (FR).
[G-5] The fusion protein of [G-4], wherein the pair of amino acid substitutions comprises substitutions of both amino acids to have the same charge or neutral charge.
[G-6] The fusion protein of [G-3] to [G-5], wherein the substitutions are selected from positions 37, 39, 44, 45, 47, 91, and 103 on the VH, and/or positions 38, 43, 44, 46, 49, 87, and 98 on the VL (according to Kabat numbering).
[G-6a] The fusion protein of [G-3] to [G-5], wherein the substitutions are selected from positions V37, Q39, G44, L45, W47, H91, Y91, and W103 on the VH, and/or positions R38, A43, P44, L46, Y49, Y87, and F98 on the VL (according to Kabat numbering).
[G-7] The fusion protein of [G-6] or [G-6a], wherein each of the positions is substituted to any of A, D, E, F, G, H, I, L, M, N, P, Q, R, S, T, V, W, or Y.
[G-8] The fusion protein of [G-7], wherein the substitution(s) is/are selected from the positions (according to Kabat numbering), comprising any one or more of the following:
Q39D,
W47A, W47L, or W47M,
Y91A, Y91L, Y91M, or H91A,
W103A, W103I, W103L, or W103M,
V37S, or V37Q,
G44Q,
L45A, or L45Q on the VH, and/or
R38E,
Y49A,
Y87A, Y87L, or Y87M,
F98A, F98L, or F98M,
A43Q,
P44A, P44S, or P44Q,
L46E, or L46Q on the VL.
[G-9] The fusion protein of any of [G-6] to [G-8], wherein the substitutions further comprise at least one modification in an amino acid present at the interface between the variable region and the ligand, wherein said amino acid residue for modification resides in the complementarity determining region (CDR).
[G-10] The fusion protein of [G-9], wherein the ligand is IL-12, the substitutions further comprise at least one modification selected from positions 30 on the VL and/or 100a on the VH (according to Kabat numbering).
[G-11] The fusion protein of [G-10], wherein the modification is a substitution selected from S30V and/or F100aI (according to Kabat numbering).
[G-12] The fusion protein of [G-11], wherein the substitutions are selected from the group consisting of any one of the following combinations (a) to (hhh) according to Kabat numbering:
(a) L46Q and Y49A on the VL;
(b) Q39D on the VH, and R38E on the VL;
(c) H91A on the VH, and L46Q and Y49A on the VL;
(d) Y91A on the VH, and A43Q and Y49A on the VL;
(e) Y91A on the VH, and P44A and Y49A on the VL;
(f) Y91A on the VH, and L46Q and Y49A on the VL;
(g) Y91A on the VH, and Y49A and Y87L on the VL;
(h) Y91M on the VH, and A43Q and Y49A on the VL;
(i) Y91M on the VH, and P44A and Y49A on the VL;
(j) Y91M on the VH, and L46Q and Y49A on the VL;
(k) Y91M on the VH, and Y49A and Y87L on the VL;
(l) Y91M on the VH, and Y49A and F98L on the VL;
(m) W103L on the VH, and A43Q and Y49A on the VL;
(n) W103L on the VH, and P44A and Y49A on the VL;
(o) W103L on the VH, and L46Q and Y49A on the VL;
(p) W103L on the VH, and Y49A and Y87L on the VL;
(q) W103I on the VH, and A43Q and Y49A on the VL;
(r) W103I on the VH, and P44A and Y49A on the VL;
(s) W103I on the VH, and L46Q and Y49A on the VL;
(t) W103M on the VH, and A43Q and Y49A on the VL;
(u) W103M on the VH, and P44A and Y49A on the VL;
(v) W103M on the VH, and L46Q and Y49A on the VL;
(w) W103M on the VH, and Y49A and Y87L on the VL;
(x) V37S on the VH, and A43Q and Y49A on the VL;
(y) V37S on the VH, and P44A and Y49A on the VL;
(z) V37S on the VH, and L46Q and Y49A on the VL;
(aa) V37S on the VH, and Y49A and Y87L on the VL;
(bb) V37S on the VH, and Y49A and F98L on the VL;
(cc) L45Q on the VH, and A43Q and Y49A on the VL;
(dd) L45Q on the VH, and P44A and Y49A on the VL;
(ee) L45Q on the VH, and L46Q and Y49A on the VL;
(ff) L45Q on the VH, and Y49A and Y87L on the VL;
(gg) L45Q on the VH, and Y49A and F98M on the VL;
(hh) F100aI on the VH, and A43Q and Y49A on the VL;
(ii) F100aI on the VH, and P44A and Y49A on the VL;
(jj) F100aI on the VH, and L46Q and Y49A on the VL;
(kk) F100aI on the VH, and Y49A and Y87L on the VL;
(ll) F100aI on the VH, and Y49A and F98L on the VL;
(mm) Y91M on the VH, and A43Q, P44A, and Y49A on the VL;
(nn) Y91M on the VH, and A43Q, L46Q, and Y49A on the VL;
(oo) Y91M on the VH, and L46Q, Y49A, and Y87M on the VL;
(pp) V37S on the VH, and L46Q, Y49A, and Y87M on the VL;
(qq) F100aI on the VH, and A43Q, L46Q, and Y49A on the VL;
(rr) F100aI on the VH, and L46Q, Y49A, and Y87M on the VL;
(ss) V37S and L45Q on the VH, and A43Q and Y49A on the VL;
(tt) V37S and Y91M on the VH, and A43Q and Y49A on the VL;
(uu) V37S and F100aI on the VH, and A43Q and Y49A on the VL;
(vv) V37S and W103M on the VH, and A43Q and Y49A on the VL;
(ww) V37S and Y91M on the VH, and L46Q and Y49A on the VL;
(xx) V37S and F100aI on the VH, and L46Q and Y49A on the VL;
(yy) V37S and L45Q on the VH, and Y49A and Y87M on the VL;
(zz) W103L on the VH, and S30V, L46Q, and Y49A on the VL;
(aaa) W103M on the VH, and S30V, L46Q, and Y49A on the VL;
(bbb) V37S and F100aI on the VH, and S30V, A43Q, and Y49A on the VL;
(ccc) V37S and F100aI on the VH, and S30V, L46Q, and Y49A on the VL;
(ddd) W103L on the VH, and S30V, Y49A, and Y87L on the VL;
(eee) V37S and F100aI on the VH, and Y49A and Y87L on the VL;
(fff) V37S and F100aI on the VH, and S30V, Y49A, and Y87L on the VL;
(ggg) V37S, F100aI, and W103M on the VH, and L46Q and Y49A on the VL; and
   (hhh) V37S, F100aI, W103L on the VH, and L46Q and Y49A on the VL.
[G-13] The fusion protein of any of [G-1] to [G-12], wherein the molecular weight of the fusion protein is smaller after protease cleavage at the protease cleavage site compared to before said cleavage.
[G-14] The fusion protein of any of [G-1]-[G-13], wherein the reduction in association between VH and VL in the cleaved state than in the uncleaved state can be represented by a percentage reduction in maximum response unit (RU) of is less than or equivalent to 15%, or is less than or equivalent to 16%, or is less than or equivalent to 17%, or is less than or equivalent to 18%, or is less than or equivalent to 19%, or is less than or equivalent to 20%, or is less than or equivalent to 21%, or is less than or equivalent to 22%, or is less than or equivalent to 23%, or is less than or equivalent to 24%, or is less than or equivalent to 25%, or is less than or equivalent to 26%, or is less than or equivalent to 27%, or is less than or equivalent to 28%, or is less than or equivalent to 29% or is less than or equivalent to 30%, or is less than or equivalent to 31%, or is less than or equivalent to 32%, or is less than or equivalent to 33%, or is less than or equivalent to 34%, or is less than or equivalent to 35%, or is less than or equivalent to 36%, or is less than or equivalent to 37%, or is less than or equivalent to 38%, or is less than or equivalent to 39%, or is less than or equivalent to 40%, as measured under surface plasma resonance (SPR) comparing RU of the fusion protein before and after protease cleavage.
[G-15] The fusion protein of any of [G-1] to [G-13], wherein the reduction in association between VH and VL in the cleaved state compared to the uncleaved state can be represented by a percentage reduction in maximum response unit (RU) of less than or equivalent to 1%, or is less than or equivalent to 2%, or is less than or equivalent to 3%, or is less than or equivalent to 4%, or is less than or equivalent to 5%, or is less than or equivalent to 6%, or is less than or equivalent to 7% or is less than or equivalent to 8%, or is less than or equivalent to 9%, or is less than or equivalent to 10%, or is less than or equivalent to 11%, or is less than or equivalent to 12%, or is less than or equivalent to 13%, or is less than or equivalent to 14%, or is less than or equivalent to 15%, or is less than or equivalent to 16%, or is less than or equivalent to 17%, or is less than or equivalent to 18%, or is less than or equivalent to 19%, or is less than or equivalent to 20%, as measured under surface plasma resonance (SPR) comparing RU of the fusion protein before and after protease cleavage.
[G-16] The fusion protein of any of [G-14] or [G-15], wherein SPR conditions comprise a contact duration of the fusion protein in the uncleaved state with 400 nM of uPA for a duration of 30 mins.
[G-17] The fusion protein of any of [G-14] to [G-16], wherein the percentage of VH-ligand or VL-ligand released is directly proportional with the percentage change in response unit (RU) of the fusion protein measured under SPR in the cleaved state compared to the uncleaved state according to formula (II):

$$VH - ligand or VL - ligand release \% = \% reduction in R U \times 100 / D , \qquad ( II )$$

wherein D corresponds to 0.01×percentage of molecular weight of VH-ligand or VL-ligand compared to the molecular weight of the fusion protein in the uncleaved state respectively.
[G-18] The fusion protein of [G-17], wherein the percentage of VH or VL released is more than or equivalent to 10%, or more than or equivalent to 20%, or more than or equivalent to 30%, or more than or equivalent to 40%, or more than or equivalent to 50%, or more than or equivalent to 60%, or more than or equivalent to 70%, or more than or equivalent to 80%, or more than or equivalent to 90%, or more than or equivalent to 100%.
[G-19] A pharmaceutical composition comprising the fusion protein of any of [G-1] to [G-18] and a pharmaceutically acceptable carrier.
[G-20] A pharmaceutical composition according to [G-19] or a fusion protein according to any of [G-1] to [G-18] for use as a medicament.
[G-21] A pharmaceutical composition according to [G-19] or a fusion protein according to any of [G-1] to [G-18] for use in a disease or disorder.
[G-22] Use of a pharmaceutical composition according to [G-19] or a fusion protein according to any of [G-1] to [G-18] in the manufacture of a medicament for the treatment of a disease or disorder.
[G-23] A method of treating an individual having a disease or disorder comprising administering an effective amount of the pharmaceutical composition according to [G-19] or a fusion protein according to any of [G-1] to [G-18].
[G-24] A isolated polynucleotide encoding the fusion protein of any of [G-1] to [G-18].
[G-25] A vector comprising the polynucleotide of [G-24].
[G-26] A host cell comprising the polynucleotide of [G-24] or the vector of [G-25].
[G-27] A method of producing the fusion protein of any of [G-1] to [G-24], comprising the step of culturing the host cell of [G-26].
[G-28] The method according to [G-27], comprising the steps of
   (a) introducing at least one amino acid modification or at least one pair of amino acid modification at the interface between VH and VL in said fusion protein, and optionally introducing at least one amino acid modification at the interface between the ligand and the variable region, that promote dissociation of VH or VL from the fusion protein,
   (b) confirming that step (a) does not disrupt binding of ligand to VH and VL,
   (c) confirming that step (a) reduced association of VH and VL upon protease cleavage at the protease cleavage site, and
   (d) linking VH or VL of step (a) with the IgG heavy chain constant region via a protease cleavage sequence,
   (e) obtaining a polynucleotide encoding said fusion protein of step (e),
   (f) culturing a host cell comprising the polynucleotide of step (e), and
   (g) producing and recovering the fusion protein from the host cell in step (f).
[G-29] The method according to [G-28], wherein the modification(s) is/are substitution(s), and the substitution(s) is/are selected from positions 37, 39, 44, 45, 47, 91, and 103 on the VH and/or positions 38, 43, 44, 46, 49, 87, and 98 on the VL (according to Kabat numbering).
[G-29a] The fusion protein of [G-28], wherein the substitution(s) is/are selected from positions V37, Q39, G44, L45, W47, H91, Y91, and W103 on the VH, and/or positions R38, A43, P44, L46, Y49, Y87, and F98 on the VL (according to Kabat numbering).
[G-30] The fusion protein of [G-29] or [G-29a], wherein each of the positions is substituted to any of A, D, E, F, G, H, I, L, M, N, P, Q, R, S, T, V, W, or Y.
[G-31] The method according to [G-30], wherein the substitution(s) is/are selected from the positions (according to Kabat numbering), comprising any one or more of the following:
Q39D,
W47A, W47L, or W47M,
Y91A, Y91L, Y91M, or H91A,
W103A, W103I, W103L, or W103M,
V37S, or V37Q,
G44Q,
L45A, or L45Q on the VH and/or
R38E,
Y49A,
Y87A, Y87L, or Y87M,
F98A, F98L, or F98M,
A43Q,
P44A, P44S, or P44Q,
L46E, or L46Q on the VL.
[G-32] The method according to [G-28] to [G-31], wherein the substitutions further comprise at least one modification in an amino acid present at the interface between the variable region and the ligand, wherein said amino acid residue for modification resides in the complementarity determining region (CDR).
[G-33] The method according to [G-32], wherein the ligand is IL-12, the substitutions further comprise at least one modification selected from positions 30 on the VL and/or 100a on the VH (according to Kabat numbering).
[G-34] The method according to [G-33], wherein the modification is a substitution selected from S30V and/or F100aI (according to Kabat numbering).
[G-35] The method according to [G-34], wherein the substitutions are selected from the group consisting of any one of the following combinations (a) to (hhh) according to Kabat numbering:
(a) L46Q and Y49A on the VL;
(b) Q39D on the VH, and R38E on the VL;
(c) H91A on the VH, and L46Q and Y49A on the VL;
(d) Y91A on the VH, and A43Q and Y49A on the VL;
(e) Y91A on the VH, and P44A and Y49A on the VL;
(f) Y91A on the VH, and L46Q and Y49A on the VL;
(g) Y91A on the VH, and Y49A and Y87L on the VL;
(h) Y91M on the VH, and A43Q and Y49A on the VL;
(i) Y91M on the VH, and P44A and Y49A on the VL;
(j) Y91M on the VH, and L46Q and Y49A on the VL;
(k) Y91M on the VH, and Y49A and Y87L on the VL;
(l) Y91M on the VH, and Y49A and F98L on the VL;
(m) W103L on the VH, and A43Q and Y49A on the VL;
(n) W103L on the VH, and P44A and Y49A on the VL;
(o) W103L on the VH, and L46Q and Y49A on the VL;
(p) W103L on the VH, and Y49A and Y87L on the VL;
(q) W103I on the VH, and A43Q and Y49A on the VL;
(r) W103I on the VH, and P44A and Y49A on the VL;
(s) W103I on the VH, and L46Q and Y49A on the VL;
(t) W103M on the VH, and A43Q and Y49A on the VL;
(u) W103M on the VH, and P44A and Y49A on the VL;
(v) W103M on the VH, and L46Q and Y49A on the VL;
(w) W103M on the VH, and Y49A and Y87L on the VL;
(x) V37S on the VH, and A43Q and Y49A on the VL;
(y) V37S on the VH, and P44A and Y49A on the VL;
(z) V37S on the VH, and L46Q and Y49A on the VL;
(aa) V37S on the VH, and Y49A and Y87L on the VL;
(bb) V37S on the VH, and Y49A and F98L on the VL;
(cc) L45Q on the VH, and A43Q and Y49A on the VL;
(dd) L45Q on the VH, and P44A and Y49A on the VL;
(ee) L45Q on the VH, and L46Q and Y49A on the VL;
(ff) L45Q on the VH, and Y49A and Y87L on the VL;
(gg) L45Q on the VH, and Y49A and F98M on the VL;
(hh) F100aI on the VH, and A43Q and Y49A on the VL;
(ii) F100aI on the VH, and P44A and Y49A on the VL;
(jj) F100aI on the VH, and L46Q and Y49A on the VL;
(kk) F100aI on the VH, and Y49A and Y87L on the VL;
(ll) F100aI on the VH, and Y49A and F98L on the VL;
(mm) Y91M on the VH, and A43Q, P44A, and Y49A on the VL;
(nn) Y91M on the VH, and A43Q, L46Q, and Y49A on the VL;
(oo) Y91M on the VH, and L46Q, Y49A, and Y87M on the VL;
(pp) V37S on the VH, and L46Q, Y49A, and Y87M on the VL;
(qq) F100aI on the VH, and A43Q, L46Q, and Y49A on the VL;
(rr) F100aI on the VH, and L46Q, Y49A, and Y87M on the VL;
(ss) V37S and L45Q on the VH, and A43Q and Y49A on the VL;
(tt) V37S and Y91M on the VH, and A43Q and Y49A on the VL;
(uu) V37S and F100aI on the VH, and A43Q and Y49A on the VL;
(vv) V37S and W103M on the VH, and A43Q and Y49A on the VL;
(ww) V37S and Y91M on the VH, and L46Q and Y49A on the VL;
(xx) V37S and F100aI on the VH, and L46Q and Y49A on the VL;
(yy) V37S and L45Q on the VH, and Y49A and Y87M on the VL;
(zz) W103L on the VH, and S30V, L46Q, and Y49A on the VL;
(aaa) W103M on the VH, and S30V, L46Q, and Y49A on the VL;
(bbb) V37S and F100aI on the VH, and S30V, A43Q, and Y49A on the VL;
(ccc) V37S and F100aI on the VH, and S30V, L46Q, and Y49A on the VL;
(ddd) W103L on the VH, and S30V, Y49A, and Y87L on the VL;
(eee) V37S and F100aI on the VH, and Y49A and Y87L on the VL;
(fff) V37S and F100aI on the VH, and S30V, Y49A, and Y87L on the VL;
(ggg) V37S, F100aI and W103M on the VH, and L46Q and Y49A on the VL; and
   (hhh) V37S, F100aI and W103L on the VH, and L46Q and Y49A on the VL.
[G-36] The method according to any of [G-27] to [G-35], further comprising recovering the polypeptide from the host cell. [H-1] A method for screening for a fusion protein or polypeptide of any of [A-1] to
[A-57], [B-1] to [B-57], [C-1] to [C-52], [D-1] to [D-5], [E-1] to [E-3], [F-1]to [F-20], [G-1] to [G-18], and [J-1] to [J-55] having mutations that reduce association between VH and VL in the cleaved state or second state compared to the uncleaved state or first state, comprising comparing the maximum response unit recorded for the fusion protein or polypeptide of any of [A-1] to [A-57], [B-1] to [B-57], [C-1] to [C-52], [D-1] to [D-5], [E-1] to [E-3], [F-1] to [F-20] and [G-1] to [G-18] before and after protease cleavage under surface plasma resonance (SPR) and selecting mutations that result in a reduction in response unit of less than or equivalent to 1%, or less than or equivalent to 2%, or less than or equivalent to 3%, or less than or equivalent to 4%, or less than or equivalent to 5%, or less than or equivalent to 6%, or less than or equivalent to 7%, or less than or equivalent to 8%, or less than or equivalent to 9%, or less than or equivalent to 10%, or less than or equivalent to 11%, or less than or equivalent to 12%, or less than or equivalent to 13%, or less than or equivalent to 14%, or less than or equivalent to 15%, or less than or equivalent to 16%, or less than or equivalent to 17%, or less than or equivalent to 18%, or less than or equivalent to 19%, or less than or equivalent to 20%, or less than or equivalent to 21%, or less than or equivalent to 22%, or less than or equivalent to 23%, or less than or equivalent to 24%, or less than or equivalent to 25%, or less than or equivalent to 26%, or less than or equivalent to 27%, or less than or equivalent to 28%, or less than or equivalent to 29% or less than or equivalent to 30%, or less than or equivalent to 31%, or less than or equivalent to 32%, or less than or equivalent to 33%, or less than or equivalent to 34%, or less than or equivalent to 35%, or less than or equivalent to 36%, or less than or equivalent to 37%, or less than or equivalent to 38%, or less than or equivalent to 39%, or less than or equivalent to 40%, before and after protease cleavage. [H-2] A method for screening for a fusion protein or polypeptide of any of [A-1] to
[A-57], [B-1] to [B-57], [C-1] to [C-52], [D-1] to [D-5], [E-1] to [E-3], [F-1] to [F-20], [G-1] to [G-18], and [J-1] to [J-55] having mutations that reduce association between VH and VL in the cleaved state or second state compared to the uncleaved state or first state, comprising the steps:
   (a) introducing at least one amino acid mutation or at least one pair of amino acid mutation at the interface between VH and VL in said fusion protein or polypeptide and optionally introducing at least one amino acid mutation at the interface between the ligand or antigen and the ligand-binding domain or antigen-binding domain, that promote dissociation of VH domain or VL domain from the fusion protein or polypeptide;
   (b) determining a first response unit (RU1) of immobilised fusion protein or polypeptide of step (a) in a BIACORE surface plasma resonance (SPR) assay in the absence of protease;
   (c) determining a second response unit (RU2) of immobilised fusion protein or polypeptide of step (a) in the same BIACORE surface plasma resonance (SPR) assay in the presence of protease;
   (d) selecting the mutation(s) in step (a) if the percentage difference between RU1 and RU2 is less than or equivalent to 1%, or is less than or equivalent to 2%, or is less than or equivalent to 3%, or is less than or equivalent to 4%, or is less than or equivalent to 5%, or is less than or equivalent to 6%, or is less than or equivalent to 7%, or is less than or equivalent to 8%, or is less than or equivalent to 9%, or is less than or equivalent to 10%, or is less than or equivalent to 11%, or is less than or equivalent to 12%, or is less than or equivalent to 13%, or is less than or equivalent to 14%, or is less than or equivalent to 15%, or is less than or equivalent to 16%, or is less than or equivalent to 17%, or is less than or equivalent to 18%, or is less than or equivalent to 19%, or is less than or equivalent to 20%, or is less than or equivalent to 21%, or is less than or equivalent to 22%, or is less than or equivalent to 23%, or is less than or equivalent to 24%, or is less than or equivalent to 25%, or is less than or equivalent to 26%, or is less than or equivalent to 27%, or is less than or equivalent to 28%, or is less than or equivalent to 29% or is less than or equivalent to 30%, or is less than or equivalent to 31%, or is less than or equivalent to 32%, or is less than or equivalent to 33%, or is less than or equivalent to 34%, or is less than or equivalent to 35%, or is less than or equivalent to 36%, or is less than or equivalent to 37%, or is less than or equivalent to 38%, or is less than or equivalent to 39%, or is less than or equivalent to 40%, before and after protease cleavage.
[H-3] The method of [H-1] or [H-2], wherein the percentage reduction in response unit corresponds to the percentage reduction in molecular weight resulting from the release of VH or VL from the fusion protein or polypeptide.
[H-4] A method for screening for a fusion protein or polypeptide of any of [A-1] to [A-57], [B-1] to [B-57], [C-1] to [C-52], [D-1] to [D-5], [E-1] to [E-3], [F-1] to [F-20], [G-1] to [G-18], and [J-1] to [J-55] having mutations that reduce association between VH and VL in the cleaved state or second state compared to the uncleaved state or first state, comprising the steps:
   (a) introducing at least one amino acid mutation or at least one pair of amino acid mutation at the interface between VH and VL in said fusion protein or polypeptide, and optionally introducing at least one amino acid mutation at the interface between the ligand or antigen and the ligand-binding domain or antigen-binding domain, that promote dissociation of VH domain or VL domain from the fusion protein or polypeptide;
   (b) subjecting a first set of the fusion protein or polypeptide before protease cleavage to Size Exclusion Chromatography (SEC) and obtaining a first chromatograph comprising peak A1 (a first peak);
   (c) subjecting a second set of the fusion protein or polypeptide after protease cleavage to SEC and obtaining a second chromatograph comprising peak A2 (a second peak) and additional peak A2′ (third peak), wherein A2′ is a shoulder peak of A2;
   (d) determining the percentage resulting from area under curve (AUC) of peak A2′ (the third peak) over AUC of peak A1 (the first peak);
   (e) selecting the mutation(s) in step (a) if the percentage obtained in step (d) is less than or equivalent to 1%, or is less than or equivalent to 2%, or is less than or equivalent to 3%, or is less than or equivalent to 4%, or is less than or equivalent to 5%, or is less than or equivalent to 6%, or is less than or equivalent to 7%, or is less than or equivalent to 8%, or is less than or equivalent to 9%, or is less than or equivalent to 10%, or is less than or equivalent to 11%, or is less than or equivalent to 12%, or is less than or equivalent to 13%, or is less than or equivalent to 14%, or is less than or equivalent to 15%, or is less than or equivalent to 16%, or is less than or equivalent to 17%, or is less than or equivalent to 18%, or is less than or equivalent to 19%, or is less than or equivalent to 20% or is less than or equivalent to 21%, or is less than or equivalent to 22%, or is less than or equivalent to 23%, or is less than or equivalent to 24%, or is less than or equivalent to 25%, or is less than or equivalent to 26%, or is less than or equivalent to 27%, or is less than or equivalent to 28%, or is less than or equivalent to 29% or is less than or equivalent to 30%, or is less than or equivalent to 31%, or is less than or equivalent to 32%, or is less than or equivalent to 33%, or is less than or equivalent to 34%, or is less than or equivalent to 35%, or is less than or equivalent to 36%, or is less than or equivalent to 37%, or is less than or equivalent to 38%, or is less than or equivalent to 39%, or is less than or equivalent to 40%.
[H-5] The method of [H-4], wherein the percentage determined in (d) corresponds to the percentage of VH or VL dissociated from the fusion protein or polypeptide after protease cleavage.
[H-6] The method of any of [H-1] to [H-5], wherein the percentage is less than or equivalent to 10% when screening for a fusion protein of any of [A-1] to [A-57], [B-1] to [B-57], [C-1] to [C-52], [D-1] to [D-5], [E-1] to [E-3], [F-1] to [F-20], [G-1] to [G-18], or [J-1] to [J-55].
[H-7] The method of any of [H-1] to [H-5], wherein the percentage is less than or equivalent to 10%, or is less than or equivalent to 16%, or is less than or equivalent to 20%, or is less than or equivalent to 30%, or is less than or equivalent to 37%, when screening for a fusion protein or polypeptide of any of [A-1] to [A-57], [B-1] to [B-57], [C-1] to [C-52], [D-1] to [D-5], [E-1] to [E-3], [F-1] to [F-20], [G-1] to [G-18], or [J-1] to [J-55].
[H-8] The method of any of [H-1] to [H-6], wherein the method further comprises the steps:
   i. determining the biological activity of a fusion protein or polypeptide of any of [A-1] to [A-57], [B-1] to [B-57], [C-1] to [C-52], [D-1] to [D-5], [E-1] to [E-3], [F-1] to [F-20], [G-1] to [G-18], [J-1] to [J-55] before protease cleavage;
   ii. determining the biological activity of a fusion protein or polypeptide of step (i) after protease cleavage;
   iii. introducing at least one amino acid modification or at least one pair of amino acid modification at the interface between VH and VL in the fusion protein or polypeptide in step (i), and optionally introducing at least one amino acid modification at the interface between the ligand or antigen and the ligand-binding domain or antigen-binding domain, wherein the amino acid modification(s) promote dissociation of VH or VL from the fusion protein or polypeptide upon protease cleavage in the presence of a protease;
   iv. determining the biological activity of the fusion protein or polypeptide in step (iii), before protease cleavage;
   v. determining the biological activity of the fusion protein or polypeptide in step (iii), after protease cleavage; and
   vi. selecting amino acid modification(s) wherein the biological activity of the fusion protein or polypeptide in step (v) is greater than the biological activity of the fusion protein or polypeptide in step (iv).
[H-9] The method of [H-8], further comprising the step of:
   (a) determining the difference in biological activity of the fusion protein or polypeptide between (i) and (ii), “V1”, and the difference in biological activity of the fusion protein or polypeptide between (iv) and (v), “V2”; and;
   (b) selecting the amino acid modification(s) wherein the value of V2 is greater than V1.
[H-10] A library of amino acid mutations that reduce association between VH and VL in a fusion protein or polypeptide of any of [A-1] to [A-57], [B-1] to [B-57], [C-1] to [C-52], [D-1] to [D-5], [E-1] to [E-3], [F-1] to [F-20], [G-1] to [G-18], and [J-1] to [J-55] comprising the mutations selected in [H-1] to [H-9].
[H-11] The library according to [H-10], wherein the mutations are substitutions selected from positions 37, 39, 44, 45, 47, 91, and 103 on the VH, and/or positions 38, 43, 44, 46, 49, 87, and 98 on the VL (according to Kabat numbering).
[H-11a] The library according to [H-10], wherein the mutations are substitutions selected from positions V37, Q39, G44, L45, W47, H91, Y91, and W103 on the VH, and/or positions R38, A43, P44, L46, Y49, Y87, and F98 on the VL (according to Kabat numbering).
[H-12] The library according to [H-11] or [H-11a], wherein each of the positions is substituted to any of A, D, E, F, G, H, I, L, M, N, P, Q, R, S, T, V, W, or Y.
[H-13] The library according to [H-12], wherein the mutation(s) is/are substitutions selected from the positions (according to Kabat numbering), comprising any one or more of the following:
Q39D,
W47A, W47L, or W47M,
Y91A, Y91L, Y91M, or H91A,
W103A, W103L, or W103M,
V37S, or V37Q,
G44Q,
L45A, or L45Q on the VH, and/or
R38E,
Y49A,
Y87A, Y87L, or Y87M,
F98A, F98L, or F98M,
A43Q,
P44A, P44S, or P44Q,
L46E, or L46Q, on the VL.
[H-14] The library according to [H-13], wherein the mutations are additionally selected from positions 30 on the VL or 100a on the VH (according to Kabat numbering).
[H-15] The library according to [H-14], wherein the mutations are substitutions selected from S30V or F100aI (according to Kabat numbering).
[H-16] The library according to [H-15], wherein the substitutions are selected from the group consisting of any one of the following combinations (a) to (hhh) according to Kabat numbering:
(a) L46Q and Y49A on the VL;
(b) Q39D on the VH, and R38E on the VL;
(c) H91A on the VH, and L46Q and Y49A on the VL;
(d) Y91A on the VH, and A43Q and Y49A on the VL;
(e) Y91A on the VH, and P44A and Y49A on the VL;
(f) Y91A on the VH, and L46Q and Y49A on the VL;
(g) Y91A on the VH, and Y49A and Y87L on the VL;
(h) Y91M on the VH, and A43Q and Y49A on the VL;
(i) Y91M on the VH, and P44A and Y49A on the VL;
(j) Y91M on the VH, and L46Q and Y49A on the VL;
(k) Y91M on the VH, and Y49A and Y87L on the VL;
(l) Y91M on the VH, and Y49A and F98L on the VL;
(m) W103L on the VH, and A43Q and Y49A on the VL;
(n) W103L on the VH, and P44A and Y49A on the VL;
(o) W103L on the VH, and L46Q and Y49A on the VL;
(p) W103L on the VH, and Y49A and Y87L on the VL;
(q) W103I on the VH, and A43Q and Y49A on the VL;
(r) W103I on the VH, and P44A and Y49A on the VL;
(s) W103I on the VH, and L46Q and Y49A on the VL;
(t) W103M on the VH, and A43Q and Y49A on the VL;
(u) W103M on the VH, and P44A and Y49A on the VL;
(v) W103M on the VH, and L46Q and Y49A on the VL;
(w) W103M on the VH, and Y49A and Y87L on the VL;
(x) V37S on the VH, and A43Q and Y49A on the VL;
(y) V37S on the VH, and P44A and Y49A on the VL;
(z) V37S on the VH, and L46Q and Y49A on the VL;
(aa) V37S on the VH, and Y49A and Y87L on the VL;
(bb) V37S on the VH, and Y49A and F98L on the VL;
(cc) L45Q on the VH, and A43Q and Y49A on the VL;
(dd) L45Q on the VH, and P44A and Y49A on the VL;
(ee) L45Q on the VH, and L46Q and Y49A on the VL;
(ff) L45Q on the VH, and Y49A and Y87L on the VL;
(gg) L45Q on the VH, and Y49A and F98M on the VL;
(hh) F100aI on the VH, and A43Q and Y49A on the VL;
(ii) F100aI on the VH, and P44A and Y49A on the VL;
(jj) F100aI on the VH, and L46Q and Y49A on the VL;
(kk) F100aI on the VH, and Y49A and Y87L on the VL;
(ll) F100aI on the VH, and Y49A and F98L on the VL;
(mm) Y91M on the VH, and A43Q, P44A, and Y49A on the VL;
(nn) Y91M on the VH, and A43Q, L46Q, and Y49A on the VL;
(oo) Y91M on the VH, and L46Q, Y49A, and Y87M on the VL;
(pp) V37S on the VH, and L46Q, Y49A, and Y87M on the VL;
(qq) F100aI on the VH, and A43Q, L46Q, and Y49A on the VL;
(rr) F100aI on the VH, and L46Q, Y49A, and Y87M on the VL;
(ss) V37S and L45Q on the VH, and A43Q and Y49A on the VL;
(tt) V37S and Y91M on the VH, and A43Q and Y49A on the VL;
(uu) V37S and F100aI on the VH, and A43Q and Y49A on the VL;
(vv) V37S and W103M on the VH, and A43Q and Y49A on the VL;
(ww) V37S and Y91M on the VH, and L46Q and Y49A on the VL;
(xx) V37S and F100aI on the VH, and L46Q and Y49A on the VL;
(yy) V37S and L45Q on the VH, and Y49A and Y87M on the VL;
(zz) W103L on the VH, and S30V, L46Q, and Y49A on the VL;
(aaa) W103M on the VH, and S30V, L46Q, and Y49A on the VL;
(bbb) V37S and F100aI on the VH, and S30V, A43Q, and Y49A on the VL;
(ccc) V37S and F100aI on the VH, and S30V, L46Q, and Y49A on the VL;
(ddd) W103L on the VH, and S30V, Y49A, and Y87L on the VL;
(eee) V37S and F100aI on the VH, and Y49A and Y87L on the VL;
(fff) V37S and F100aI on the VH, and S30V, Y49A, and Y87L on the VL;
(ggg) V37S, F100aI and W103M on the VH, and L46Q and Y49A on the VL; and
   (hhh) V37S, F100aI and W103L on the VH, and L46Q and Y49A on the VL.
[I-1] An isolated protease-resistant Interluekin-12 (IL-12).
[I-2] The protease-resistant IL-12 of [I-1], wherein the protease is selected from the group consisting of: matriptase, urokinase-type plasminogen activator (uPA) and matrix metalloprotease (MMP).
[I-3] The protease-resistant IL-12 of [I-2], wherein the protease is urokinase-type plasminogen activator (uPA).
[I-4] The protease-resistant IL-12 of any of [I-1] to [I-3], comprising at least one amino acid modification that prevents proteolytic degradation of IL-12 when exposed to protease.
[I-5] The protease-resistant IL-12 of [I-4], which does not comprise the amino acid sequence of KSKREK (SEQ ID NO: 1102).
[I-6] The protease-resistant IL-12 of [I-5], wherein the at least one amino acid modification is performed at the interface between IL-12 and heparin binding site of IL-12.
[I-7] The protease-resistant IL-12 of [I-6], wherein after performing the at least one amino acid modification, the IL-12 comprises a modified sequence selected from the group consisting of (a) to (p):

(SEQ ID NO: 1052)
(a) KSHRE;
(SEQ ID NO: 1053)
(b) KSHHE;
(SEQ ID NO: 1054)
(c) KSHKE;
(SEQ ID NO: 1055)
(d) KSHSE;
(SEQ ID NO: 1056)
(e) KSKHRE;
(SEQ ID NO: 1057)
(f) KSKQRE;
(SEQ ID NO: 1058)
(g) KSKERE;
(SEQ ID NO: 1059)
(h) KSKPRE;
(SEQ ID NO: 1060)
(i) KHKE;
(SEQ ID NO: 1061)
(j) KHHE;
(SEQ ID NO: 1062)
(k) KHRE;
(SEQ ID NO: 1063)
(l) KKHE;
(SEQ ID NO: 1064)
(m) KRHE;
(SEQ ID NO: 1065)
(n) KRE;
(SEQ ID NO: 1066)
(o) KHE;
and
(SEQ ID NO: 1067)
(p) KKE.

[I-8] The protease-resistant IL-12 of any of [I-1] to [I-7], wherein the IL-12 comprises any of the following (i) to (xvi):
   (i) an amino acid sequence that is at least 70%, 80%, or 90% identical to SEQ ID NO: 1068;
   (ii) an amino acid sequence that is at least 70%, 80%, or 90% identical to SEQ ID NO: 1069;
   (iii) an amino acid sequence that is at least 70%, 80%, or 90% identical to SEQ ID NO: 1070;
   (iv) an amino acid sequence that is at least 70%, 80%, or 90% identical to SEQ ID NO: 1071;
   (v) an amino acid sequence that is at least 70%, 80%, or 90% identical to SEQ ID NO: 1072;
   (vi) an amino acid sequence that is at least 70%, 80%, or 90% identical to SEQ ID NO: 1073;
   (vii) an amino acid sequence that is at least 70%, 80%, or 90% identical to SEQ ID NO: 1074;
   (viii) an amino acid sequence that is at least 70%, 80%, or 90% identical to SEQ ID NO: 1075;
   (ix) an amino acid sequence that is at least 70%, 80%, or 90% identical to SEQ ID NO: 1076;
   (x) an amino acid sequence that is at least 70%, 80%, or 90% identical to SEQ ID NO: 1077;
   (xi) an amino acid sequence that is at least 70%, 80%, or 90% identical to SEQ ID NO: 1078;
   (xii) an amino acid sequence that is at least 70%, 80%, or 90% identical to SEQ ID NO: 1079;
   (xiii) an amino acid sequence that is at least 70%, 80%, or 90% identical to SEQ ID NO: 1080;
   (xiv) an amino acid sequence that is at least 70%, 80%, or 90% identical to SEQ ID NO: 1081;
   (xv) an amino acid sequence that is at least 70%, 80%, or 90% identical to SEQ ID NO: 1082; and
   (xvi) an amino acid sequence that is at least 70%, 80%, or 90% identical to SEQ ID NO: 1083.
[I-9] The protease-resistant IL-12 of any of [I-1] to [I-8], wherein the IL-12 comprises any of the following (i) to (xvi):
   (i) an amino acid sequence that is identical to SEQ ID NO: 1068;
   (ii) an amino acid sequence that is identical to SEQ ID NO: 1069;
   (iii) an amino acid sequence that is identical to SEQ ID NO: 1070;
   (iv) an amino acid sequence that is identical to SEQ ID NO: 1071;
   (v) an amino acid sequence that is identical to SEQ ID NO: 1072;
   (vi) an amino acid sequence that is identical to SEQ ID NO: 1073;
   (vii) an amino acid sequence that is identical to SEQ ID NO: 1074;
   (viii) an amino acid sequence that is identical to SEQ ID NO: 1075;
   (ix) an amino acid sequence that is identical to SEQ ID NO: 1076;
   (x) an amino acid sequence that is identical to SEQ ID NO: 1077;
   (xi) an amino acid sequence that is identical to SEQ ID NO: 1078;
   (xii) an amino acid sequence that is identical to SEQ ID NO: 1079;
   (xiii) an amino acid sequence that is identical to SEQ ID NO: 1080;
   (xiv) an amino acid sequence that is identical to SEQ ID NO: 1081;
   (xv) an amino acid sequence that is identical to SEQ ID NO: 1082; and
   (xvi) an amino acid sequence that is identical to SEQ ID NO: 1083.
[J-1] A bivalent homodimer fusion protein comprising two polypeptides, each represented by the general formula (I), from the N- to the C-terminus:

[ligand-binding domain]-[Lx]-[Cx]-[Ly]-[ligand moiety] (I)
wherein:
Lx represents a peptide linker comprising a protease cleavage site,
Cx represents a constant region comprising a second peptide linker and optionally one or more amino acid residues which are modified from or to cysteine;
Ly represents a third peptide linker,
and wherein the ligand-binding domain comprises a heavy chain variable domain (VH) and a light chain variable domain (VL), and wherein the ligand-binding domain comprises at least one amino acid modification that reduces association between VH and VL in the presence of a protease that would catalyse the cleavage of said protease cleavage site (“cleaved state”) compared to in the absence of said protease (“uncleaved state”).
[J-2] The fusion protein of [J-1], wherein the modification is a substitution of an amino acid present at the interface between the VH and the VL, and wherein said amino acid residue for modification resides in the Framework region (FR).
[J-3] The fusion protein of [J-2], wherein the substitution(s) is/are selected from positions 37, 45, 91 or 103 on the VH, and/or positions 43, 46, 49 or 87 on the VL (according to Kabat numbering).
[J-3a] The fusion protein of [J-2], wherein the substitution(s) is/are selected from positions V37, L45, H91, Y91 or W103 on the VH, and/or positions A43, L46, Y49 or Y87 on the VL (according to Kabat numbering).
[J-4] The fusion protein of [J-3] or [J-3a], wherein each of the positions is substituted to any of A, D, E, F, G, H, I, L, M, N, P, Q, R, S, T, V, W, or Y.
[J-5] The fusion protein of [J-4], wherein the substitution(s) is/are selected from the positions (according to Kabat numbering), comprising any one or more of the following:
V37S,
L45Q,
Y91M, or H91A,
W103I, W103L or W103M on the VH, and/or
A43Q,
L46Q,
Y49A or
Y87L on the VL.
[J-6] The fusion protein of [J-5], wherein the substitutions further comprise at least one modification in an amino acid present at the interface between the ligand-binding domain and the ligand, wherein said amino acid residue for modification resides in the complementarity determining region (CDR).
[J-7] The fusion protein of [J-6], wherein the ligand moiety is IL-12, the substitutions further comprise at least one modification selected from positions 30 on the VL and/or 100a on the VH (according to Kabat numbering).
[J-8] The fusion protein of [J-7], wherein the modification is a substitution selected from S30V and/or F100aI (according to Kabat numbering).
[J-9] The fusion protein of [J-2] to [J-8], wherein the substitutions are selected from the group consisting of any one of the following combinations (a) to (z) according to Kabat numbering:
(a) L46Q and Y49A on the VL;
(b) H91A on the VH, and L46Q and Y49A on the VL;
(c) Y91M on the VH, and A43Q and Y49A on the VL;
(d) Y91M on the VH, and A43Q, L46Q, and Y49A on the VL;
(e) W103M on the VH, and A43Q and Y49A on the VL;
(f) W103M on the VH, and L46Q and Y49A on the VL;
(g) V37S on the VH, and A43Q and Y49A on the VL;
(h) V37S on the VH, and L46Q and Y49A on the VL;
(i) L45Q on the VH, and A43Q and Y49A on the VL;
(j) L45Q on the VH, and L46Q and Y49A on the VL;
(k) F100aI on the VH, and A43Q and Y49A on the VL;
(l) F100aI on the VH, and A43Q, L46Q, and Y49A on the VL;
(m) W103L on the VH, and S30V, L46Q, and Y49A on the VL;
(n) W103M on the VH, and S30V, L46Q, and Y49A on the VL;
(o) V37S and F100aI on the VH, and S30V, A43Q, and Y49A on the VL;
(p) V37S and F100aI on the VH, and S30V, L46Q, and Y49A on the VL;
(q) W103L on the VH, and L46Q and Y49A on the VL;
(r) W103I on the VH, and L46Q and Y49A on the VL;
(s) W103M on the VH, and Y49A and Y87L on the VL;
(t) W103L on the VH, and Y49A and Y87L on the VL;
(u) W103L on the VH, and S30V, Y49A, and Y87L on the VL;
(v) V37S and F100aI on the VH, and L46Q and Y49A on the VL;
(w) V37S and F100aI on the VH, and Y49A and Y87L on the VL; and
(x) V37S and F100aI on the VH, and S30V, Y49A, and Y87L on the VL;
(y) V37S, F100aI and W103M on the VH, and L46Q and Y49A on the VL; and
   (z) V37S, F100aI and W103L on the VH, and L46Q and Y49A on the VL.
[J-10] The fusion protein of [J-9], wherein the substitutions are selected from the group consisting of any one of the following combinations (a) to (g) according to Kabat numbering:
(a) W103M on the VH, and L46Q and Y49A on the VL;
(b) W103L on the VH, and S30V, L46Q, and Y49A on the VL;
(c) V37S and F100aI on the VH, and S30V, L46Q, and Y49A on the VL;
(d) W103L on the VH, and L46Q and Y49A on the VL; and
(e) V37S and F100aI on the VH, and L46Q and Y49A on the VL;
(f) V37S, F100aI and W103M on the VH, and L46Q and Y49A on the VL; and
   (g) V37S, F100aI and W103L on the VH, and L46Q and Y49A on the VL.
[J-11] The fusion protein of any of [J-1]-[J-10], wherein the molecular weight of the fusion protein in the cleaved state is smaller than the molecular weight of the fusion protein in the uncleaved state.
[J-12] The fusion protein of any of [J-1]-[J-11], wherein in the cleaved state, the cleavage site is cleaved so that a portion of the ligand-binding domain is released from the fusion protein.
[J-13] The fusion protein of [J-12], wherein the molecular weight of the portion of ligand-binding domain released from the fusion protein is 26 kDa, or 13 kDa, or smaller.
[J-14] The fusion protein of any of [J-1]-[J-13] wherein the ratio of the molecular weight of the fusion protein in the cleaved state and the molecular weight of the fusion protein in the uncleaved state is 10:9.
[J-15] The fusion protein of [J-1]-[J-14], wherein the molecular weight of the fusion protein in the cleaved state is 9/10 that of the molecular weight of the fusion protein in the uncleaved state.
[J-16] The fusion protein of any of [J-1]-[J-15], wherein the percentage reduction in molecular weight of the fusion protein in the cleaved state compared to the fusion protein in the uncleaved state is 10%.
[J-17] The fusion protein of any of [J-1]-[J-16], wherein the portion of ligand-binding domain released from the fusion protein upon protease cleavage comprises VL or VH.
[J-18] The fusion protein of any of [J-1]-[J-17], wherein the reduction in association between VH and VL in the cleaved state than in the uncleaved state can be represented by a percentage reduction in maximum response unit (RU) of is less than or equivalent to 1%, or is less than or equivalent to 2%, or is less than or equivalent to 3%, or is less than or equivalent to 4%, or is less than or equivalent to 5%, or is less than or equivalent to 6%, or is less than or equivalent to 7% or is less than or equivalent to 8%, or is less than or equivalent to 9%, or is less than or equivalent to 10%, or is less than or equivalent to 11%, or is less than or equivalent to 12%, or is less than or equivalent to 13%, or is less than or equivalent to 14%, or is less than or equivalent to 15%, or is less than or equivalent to 16%, or is less than or equivalent to 17%, or is less than or equivalent to 18%, or is less than or equivalent to 19%, or is less than or equivalent to 20%, as measured under surface plasma resonance (SPR) comparing RU of the fusion protein in the absence of a protease and in the presence of a protease.
[J-19] The fusion protein of any of [J-1]-[J-18], wherein the reduction in association between VH and VL in the cleaved state than in the uncleaved state can be represented by a percentage reduction in maximum response unit (RU) of is less than or equivalent to 1%, or is less than or equivalent to 2%, or is less than or equivalent to 3%, or is less than or equivalent to 4%, or is less than or equivalent to 5%, or is less than or equivalent to 6%, or is less than or equivalent to 7%, as measured under surface plasma resonance (SPR) comparing RU of the fusion protein in the absence of a protease and in the presence of a protease.
[J-20] The fusion protein of any of [J-1]-[J-19], wherein the reduction in association between VH and VL in the cleaved state than in the uncleaved state can be represented by a percentage reduction in maximum response unit (RU) of is less than or equivalent to 15%, or is less than or equivalent to 16%, or is less than or equivalent to 17%, or is less than or equivalent to 18%, or is less than or equivalent to 19%, or is less than or equivalent to 20%, or is less than or equivalent to 21%, or is less than or equivalent to 22%, or is less than or equivalent to 23%, or is less than or equivalent to 24%, or is less than or equivalent to 25%, or is less than or equivalent to 26%, or is less than or equivalent to 27%, or is less than or equivalent to 28%, or is less than or equivalent to 29%, or is less than or equivalent to 30%, or is less than or equivalent to 31%, or is less than or equivalent to 32%, or is less than or equivalent to 33%, or is less than or equivalent to 34%, or is less than or equivalent to 35%, or is less than or equivalent to 36%, or is less than or equivalent to 37%, or is less than or equivalent to 38%, or is less than or equivalent to 39%, or is less than or equivalent to 40%, as measured under surface plasma resonance (SPR) comparing RU of the fusion protein in the absence of a protease and in the presence of a protease.
[J-21] The fusion protein of any of [J-1]-[J-20], wherein SPR conditions comprise a contact duration of the fusion protein in the uncleaved state with 400 nM of uPA protease for a duration of 30 mins.
[J-22] The fusion protein of any of [J-19] to [J-20], wherein the percentage of VH or VL released is directly proportional with the percentage change in response unit (RU) of the fusion protein measured under SPR in the cleaved state compared to the uncleaved state according to formula (II):

$$VH or VL release \% = \% reduction in R U \times 100 / D , \qquad ( II )$$

wherein D corresponds to 0.01×percentage of molecular weight of VH or VL compared to the molecular weight of the fusion protein in the uncleaved state respectively.
[J-23] The fusion protein of [J-22], wherein the percentage of VH or VL released is directly proportional with the percentage change in response unit (RU) of the fusion protein measured under SPR in the cleaved state compared to the uncleaved state according to formula (II-1):

$$VH or VL release \% = \% reduction in RU \times 100 / 10. \qquad ( II - 1 )$$

[J-24] The fusion protein of [J-23], wherein the percentage of VH or VL released is directly proportional with the percentage change in response unit (RU) of the fusion protein measured under SPR in the cleaved state compared to the uncleaved state according to formula (II-2):

$$VH or VL release \% = \% reduction in RU \times 100 / 1 \; 5.8 . \qquad ( II - 2 )$$

[J-25] The fusion protein of any one of [J-22] to [J-24], wherein the percentage of VH or VL released is more than or equivalent to 10%, or more than or equivalent to 20%, or more than or equivalent to 30%, or more than or equivalent to 40%, or more than or equivalent to 50%, or more than or equivalent to 60%, or more than or equivalent to 70%, or more than or equivalent to 80%, or more than or equivalent to 90%, or more than or equivalent to 100%.
[J-26] The fusion protein of any of [J-1] to [J-25], wherein the ligand moiety in the uncleaved and cleaved state remains bound to the constant region via the third peptide linker.
[J-27] The fusion protein of any of [J-1] to [J-26], wherein binding between ligand moiety and ligand-binding domain is attenuated in the cleaved state compared to in the uncleaved state.
[J-28] The fusion protein of any of [J-1] to [J-27], wherein in the uncleaved state, the ligand moiety is bound by the ligand-binding domain and the biological activity of the ligand moiety is attenuated, and in the cleaved state, the biological activity of the ligand is restored.
[J-29] The fusion protein of any of [J-1] to [J-28], wherein Cx comprises a CH1 region of the heavy chain and a CL region of the light chain.
[J-30] The fusion protein of any of [J-1]-[J-29], wherein the second peptide linker is positioned in the hinge region so that disulphide bond formation between Cys at position 220 (C220) of the heavy chain and Cys at position 214 (C214) of the light chain (according to EU numbering) is promoted.
[J-31] The fusion protein of any of [J-1]-[J-29], wherein Cx comprises at least one amino acid modification wherein amino acid residues in the heavy chain and the light chain are modified so that no disulphide bond is formed between position 220 of the heavy chain and position 214 of the light chain (according to EU numbering).
[J-32] The fusion protein of [J-31], wherein the light chain comprises C214S modification and the heavy chain comprises C220S modification (according to EU numbering).
[J-33] The fusion protein of any of [J-1]-[J-29], wherein the heavy chain is modified to allow disulphide bond formation between position 131 of the heavy chain and position 214 of the light chain (according to EU numbering).
[J-34] The fusion protein of [J-33], wherein the heavy chain comprises S131C and C220S modifications (according to EU numbering).
[J-35] The fusion protein of any of [J-1] to [J-34], wherein Cx comprises a sequence selected from the group consisting of SEQ ID NO: 901 (C1), SEQ ID NO: 905 (C2), SEQ ID NO: 908 (C3), SEQ ID NO: 910 (C4) and SEQ ID NO: 932 (C5).
[J-36] The fusion protein of [J-35], wherein Cx comprises a sequence of SEQ ID NO: 910 (C4).
[J-37] The fusion protein of any of [J-1] to [J-36], wherein Ly comprises a glycine-serine polymer.
[J-38] The fusion protein of [J-37], wherein the glycine-serine polymer is selected from the group consisting of (a) to (ee):

| | |
|---|---|
| (a) | Ser; |
| (b) | Gly Ser (GS); |
| (c) | Ser Gly (SG); |
| (d) | Gly Gly Ser (GGS); |
| (e) | Gly Ser Gly (GSG); |
| (f) | Ser Gly Gly (SGG); |
| (g) | Gly Ser Ser (GSS); |
| (h) | Ser Ser Gly (SSG); |
| (i) | Ser Gly Ser (SGS); |
| (j) | Gly Gly Gly Ser (GGGS, SEQ ID NO: 136); |
| (k) | Gly Gly Ser Gly (GGSG, SEQ ID NO: 137); |
| (l) | Gly Ser Gly Gly (GSGG, SEQ ID NO: 138); |
| (m) | Ser Gly Gly Gly (SGGG, SEQ ID NO: 139); |
| (n) | Gly Ser Ser Gly (GSSG, SEQ ID NO: 140); |
| (o) | Gly Gly Gly Gly Ser (GGGGS, SEQ ID NO: 141); |
| (p) | Gly Gly Gly Ser Gly (GGGSG, SEQ ID NO: 142); |
| (q) | Gly Gly Ser Gly Gly (GGSGG, SEQ ID NO: 143); |
| (r) | Gly Ser Gly Gly Gly (GSGGG, SEQ ID NO: 144); |
| (s) | Gly Ser Gly Gly Ser (GSGGS, SEQ ID NO: 145); |
| (t) | Ser Gly Gly Gly Gly (SGGGG, SEQ ID NO: 146); |
| (u) | Gly Ser Ser Gly Gly (GSSGG, SEQ ID NO: 147); |
| (v) | Gly Ser Gly Ser Gly (GSGSG, SEQ ID NO: 148); |
| (w) | Ser Gly Gly Ser Gly (SGGSG, SEQ ID NO: 149); |
| (x) | Gly Ser Ser Ser Gly (GSSSG, SEQ ID NO: 150); |
| (y) | Gly Gly Gly Gly Gly Ser (GGGGGS, SEQ ID NO: |
| | 151); |
| (z) | Ser Gly Gly Gly Gly Gly (SGGGGG, SEQ ID NO: |
| | 152); |
| (aa) | Gly Gly Gly Gly Gly Gly Ser (GGGGGGS, SEQ ID |
| | NO: 153); |
| (bb) | Ser Gly Gly Gly Gly Gly Gly (SGGGGGG, SEQ ID |
| | NO: 154); |
| (cc) | (Gly Gly Gly Gly Ser (GGGGS, SEQ ID NO: |
| | 141))n; |
| (dd) | (Ser Gly Gly Gly Gly (SGGGG, SEQ ID NO: |
| | 146))n; |
| and | |
| (ee) | (Gly Gly Ser Gly Gly (GGSGG, SEQ ID NO: |
| | 143))n; |

wherein n is an integer of 1 or larger.
[J-39] The fusion protein of [J-38], wherein Ly comprises a sequence of GGSGGSGGSGGSGGSGGS (SEQ ID NO: 903).

(SEQ ID NO: 903)
GGSGGSGGSGGSGGSGGS.

[J-40] The fusion protein of any of [J-1] to [J-39], wherein the fusion protein comprises two protease cleavage sites, and wherein each protease cleavage site is independently cleavable by a protease specific to a target tissue.
[J-41] The fusion protein of [J-40] wherein the target tissue is a cancer tissue or inflammatory tissue.
[J-42] The fusion protein of any of [J-1] to [J-41], wherein each protease cleavage site is cleavable by the same protease.
[J-43] The fusion protein of [J-42], wherein each protease cleavage site comprises the same protease cleavage sequence.
[J-44] The fusion protein of any of [J-1] to [J-43] wherein each protease cleavage site is independently cleavable by a protease selected from the group consisting of matriptase, urokinase-type plasminogen activator (uPA) and matrix metalloprotease (MMP).
[J-45] The fusion protein of any of [J-1] to [J-44], wherein Lx comprises a protease cleavage site which is located near the boundary between the VH and CH1 region or the VL and CL region.
[J-46] The fusion protein of any of [J-1] to [J-45], wherein the ligand moiety comprises a cytokine or a chemokine.
[J-47] The fusion protein of [J-46], wherein the ligand moiety is selected from the group consisting of CXCL9, CXCL10, CXCL11, IL-2, IL-7, IL-12, IL-15, IL-18, IL-21, IL-22, IFN-alpha, IFN-beta, IFN-gamma, MIG, I-TAC, RANTES, MIP-1a, MIP-1b, IL-1R1, IL-1R2, IL-1RAcP and IL-1Ra.
[J-48] The fusion protein of [J-47], wherein the ligand moiety is IL-12.
[J-49] The fusion protein of [J-48], wherein the IL-12 comprises at least one amino acid modification that prevents proteolytic degradation when exposed to a protease that catalyses the cleavage of IL-12.
[J-50] The fusion protein of [J-49], wherein the IL-12 does not comprise the amino acid sequence of KSKREK (SEQ ID NO: 1102).
[J-51] The fusion protein of [J-49] or [J-50], wherein the at least one amino acid modification is performed at the interface between IL-12 and the ligand-binding domain.
[J-52] The fusion protein of [J-51], wherein after performing the at least one amino acid modification, the IL-12 comprises a modified sequence selected from the group consisting of (a) to (p):

(SEQ ID NO: 1052)
(a) KSHRE;
(SEQ ID NO: 1053)
(b) KSHHE;
(SEQ ID NO: 1054)
(c) KSHKE;
(SEQ ID NO: 1055)
(d) KSHSE;
(SEQ ID NO: 1056)
(e) KSKHRE;
(SEQ ID NO: 1057)
(f) KSKQRE;
(SEQ ID NO: 1058)
(g) KSKERE;
(SEQ ID NO: 1059)
(h) KSKPRE;
(SEQ ID NO: 1060)
(i) KHKE;
(SEQ ID NO: 1061)
(j) KHHE;
(SEQ ID NO: 1062)
(k) KHRE;
(SEQ ID NO: 1063)
(l) KKHE;
(SEQ ID NO: 1064)
(m) KRHE;
(SEQ ID NO: 1065)
(n) KRE;
(SEQ ID NO: 1066)
(o) KHE;
and
(SEQ ID NO: 1067)
(p) KKE.

[J-53] The fusion protein of [J-49] to [J-52], wherein the IL-12 comprises the sequence selected from the group consisting of (i) to (xvi):
   (i) an amino acid sequence that is at least 70%, 80%, or 90% identical to SEQ ID NO: 1068;
   (ii) an amino acid sequence that is at least 70%, 80%, or 90% identical to SEQ ID NO: 1069;
   (iii) an amino acid sequence that is at least 70%, 80%, or 90% identical to SEQ ID NO: 1070;
   (iv) an amino acid sequence that is at least 70%, 80%, or 90% identical to SEQ ID NO: 1071;
   (v) an amino acid sequence that is at least 70%, 80%, or 90% identical to SEQ ID NO: 1072;
   (vi) an amino acid sequence that is at least 70%, 80%, or 90% identical to SEQ ID NO: 1073;
   (vii) an amino acid sequence that is at least 70%, 80%, or 90% identical to SEQ ID NO: 1074;
   (viii) an amino acid sequence that is at least 70%, 80%, or 90% identical to SEQ ID NO: 1075;
   (ix) an amino acid sequence that is at least 70%, 80%, or 90% identical to SEQ ID NO: 1076;
   (x) an amino acid sequence that is at least 70%, 80%, or 90% identical to SEQ ID NO: 1077;
   (xi) an amino acid sequence that is at least 70%, 80%, or 90% identical to SEQ ID NO: 1078;
   (xii) an amino acid sequence that is at least 70%, 80%, or 90% identical to SEQ ID NO: 1079;
   (xiii) an amino acid sequence that is at least 70%, 80%, or 90% identical to SEQ ID NO: 1080;
   (xiv) an amino acid sequence that is at least 70%, 80%, or 90% identical to SEQ ID NO: 1081;
   (xv) an amino acid sequence that is at least 70%, 80%, or 90% identical to SEQ ID NO: 1082; and
   (xvi) an amino acid sequence that is at least 70%, 80%, or 90% identical to SEQ ID NO: 1083.
[J-54] The fusion protein of [J-53], wherein the IL-12 comprises the sequence selected from the group consisting of (i) to (xvi):
   (i) an amino acid sequence that is identical to SEQ ID NO: 1068;
   (ii) an amino acid sequence that is identical to SEQ ID NO: 1069;
   (iii) an amino acid sequence that is identical to SEQ ID NO: 1070;
   (iv) an amino acid sequence that is identical to SEQ ID NO: 1071;
   (v) an amino acid sequence that is identical to SEQ ID NO: 1072;
   (vi) an amino acid sequence that is identical to SEQ ID NO: 1073;
   (vii) an amino acid sequence that is identical to SEQ ID NO: 1074;
   (viii) an amino acid sequence that is identical to SEQ ID NO: 1075;
   (ix) an amino acid sequence that is identical to SEQ ID NO: 1076;
   (x) an amino acid sequence that is identical to SEQ ID NO: 1077;
   (xi) an amino acid sequence that is identical to SEQ ID NO: 1078;
   (xii) an amino acid sequence that is identical to SEQ ID NO: 1079;
   (xiii) an amino acid sequence that is identical to SEQ ID NO: 1080;
   (xiv) an amino acid sequence that is identical to SEQ ID NO: 1081;
   (xv) an amino acid sequence that is identical to SEQ ID NO: 1082; and
   (xvi) an amino acid sequence that is identical to SEQ ID NO: 1083.
[J-55] The fusion protein of [J-54], wherein the IL-12 comprises the sequence selected from SEQ ID NO: 1068, or SEQ ID NO: 1069, or SEQ ID NO: 1076, or SEQ ID NO: 1077, or SEQ ID NO: 1078, or SEQ ID NO: 1079, or SEQ ID NO: 1080.
[K-1] A library comprising a fusion protein of any of the preceding embodiments, wherein the library is obtained by a method of screening for fusion proteins comprising one or more amino acid modifications that reduce association between VH and VL in the presence of a protease compared to in the absence of a protease, wherein the method of screening is as exemplified in any of the preceding embodiments.
[K-2] A library comprising a fusion protein of any of the preceding embodiments, wherein the library is obtained by a method of producing fusion proteins comprising one or more amino acid modifications that reduce association between VH and VL in the presence of a protease compared to in the absence of a protease, wherein the one or more amino acid modifications that reduce association between VH and VL in the presence of a protease compared to in the absence of a protease is identified by a method of screening as exemplified in any of the preceding embodiments.
[K-3] A method of releasing VH or VL from a fusion protein of any of the preceding embodiments, or a polypeptide of any of the preceding embodiments, comprising a step of introducing at least one amino acid modification at the interface between VH and VL that reduces association between VH and VL, and wherein the at least one amino acid modification is/are selected from a method of screening exemplified in any of the preceding embodiments.
[K-4] A library comprising a plurality of bivalent homodimer fusion proteins, wherein each fusion protein within the library comprises a protease cleavage site and a ligand-binding domain, wherein the ligand-binding domain comprises a heavy chain variable domain (VH) and a light chain variable domain (VL) that associates with each other, and wherein the ligand-binding domain comprises at least one amino acid modification(s) that reduces the association between VH and VL before and after protease cleavage at said cleavage site.
[K-5] A method of releasing VH or VL from a bivalent homodimer fusion protein, wherein the fusion protein comprises a protease cleavage site and a ligand-binding domain, wherein the ligand-binding domain comprises a heavy chain variable domain (VH) and a light chain variable domain (VL) that associates with each other, and wherein the ligand-binding domain comprises at least one amino acid modification(s) that reduces the association between VH and VL after protease cleavage at said cleavage site compared to before protease cleavage at said cleavage site, and wherein said VH or VL is released from the fusion protein after protease cleavage at said cleavage site, the method comprising a step of introducing at least one amino acid modification(s) at the interface between VH and VL, and wherein said amino acid(s) reside(s) in the Framework region (FR).
[K-6] A method of screening for a bivalent homodimer fusion protein, wherein the fusion protein comprises a protease cleavage site and a ligand-binding domain, wherein the ligand-binding domain comprises a heavy chain variable domain (VH) and a light chain variable domain (VL) that associates with each other, and wherein the ligand-binding domain comprises at least one amino acid modification(s) that reduces the association between VH and VL after protease cleavage at said cleavage site (“cleaved state”) compared to before protease cleavage at said cleavage site (“uncleaved state”), and wherein said VH or VL is released from the fusion protein after protease cleavage at said cleavage site, and wherein the method comprises the steps:
   (a) introducing at least one amino acid modification or at least one pair of amino acid modification at the interface between VH and VL, and optionally introducing at least one amino acid modification at the interface between the ligand and the ligand-binding domain, that promote dissociation of VH or VL;
   (b) determining a first response unit (RU1) of immobilised fusion protein of step (a) in a BIACORE surface plasma resonance (SPR) assay in the uncleaved state;
   (c) determining a second response unit (RU2) of immobilised fusion protein of step (a) in the same BIACORE surface plasma resonance (SPR) assay in the cleaved state; and
   (d) selecting the modification(s) in step (a) if the percentage difference between RU1 and RU2 is less than or equivalent to 1%, or is less than or equivalent to 5%, or is less than or equivalent to 10%, or is less than or equivalent to 15%, or is less than or equivalent to 20%, or is less than or equivalent to 30%, or is less than or equivalent to 40%, and wherein the percentage reduction in response unit corresponds to the percentage reduction in molecular weight resulting from the release of VH or VL from the fusion protein.
[K-7] A method of screening for a bivalent homodimer fusion protein, wherein the fusion protein comprises a protease cleavage site and a ligand-binding domain, wherein the ligand-binding domain comprises a heavy chain variable domain (VH) and a light chain variable domain (VL) that associates with each other, and wherein the ligand-binding domain comprises at least one amino acid modification(s) that reduces the association between VH and VL after protease cleavage at said cleavage site (“cleaved state”) compared to before protease cleavage at said cleavage site (“uncleaved state”), and wherein said VH or VL is released from the fusion protein after protease cleavage at said cleavage site, and wherein the method comprises the steps:
   (a) introducing at least one amino acid modification or at least one pair of amino acid modification at the interface between VH and VL, and optionally introducing at least one amino acid modification at the interface between the ligand and the ligand-binding domain, that promote dissociation of VH or VL;
   (b) subjecting a first set of the fusion protein in the uncleaved state to Size Exclusion Chromatography (SEC) and obtaining a first chromatograph comprising peak A1 (a first peak);
   (c) subjecting a second set of the fusion protein in the cleaved state to SEC and obtaining a second chromatograph comprising peak A2 (a second peak) and additional peak A2′ (third peak), wherein A2′ is a shoulder peak of A2;
   (d) determining the percentage resulting from area under curve (AUC) of peak A2′ (third peak) over AUC of peak A1 (the first peak); and
   (e) selecting the modification(s) in step (a) wherein percentage obtained in step (d) is less than or equivalent to 1%, or is less than or equivalent to 5%, or is less than or equivalent to 10%, or is less than or equivalent to 15%, or is less than or equivalent to 20%, or is less than or equivalent to 30%, or is less than or equivalent to 40%, and
   wherein the percentage reduction determined in step (d) corresponds to the percentage reduction in molecular weight resulting from the release of VH or VL from the fusion protein.

### BRIEF DESCRIPTION OF DRAWINGS

FIG. 1. Expected Profile of IL-12 fusion proteins. As inactive molecule, biological activity of IL-12 should be inhibited and the IL-12 fusion protein should have long systemic half-life. As an activated molecule, due to cleavage by disease specific proteases, IL-12 biological activity is restored. In addition, the fusion protein should retain in high concentrations in disease tissue and should exhibit short systemic half-life.

FIG. 2. FIG. 2A shows the molecular format of monovalent IL-12 fusion proteins used to evaluate the effect of ligand binding domain on the pharmacokinetics of the inactive IL-12 fusion proteins. FIG. 2B shows the pharmacokinetics of inactive IL-12 fusion protein in non-tumour bearing mice. Upper graph shows plasma concentrations of monovalent IL-12 release FP1 (closed circle), FP2 (closed triangle), and FP3 (cross mark) after a single intravenous dose in non-tumour bearing mice (n=3). Lower table shows pharmacokinetic parameters of each fusion protein. C₀: back-extrapolated concentration immediately after intravenous injection, t_{1/2}: elimination half-life, AUC_{inf}: area under the plasma concentration-time curve from time zero extrapolated to infinity, CL: total clearance, Vₛₛ: volume of distribution at steady state.

FIG. 3. Different formats of IL-12 fusion proteins. FIG. 3A shows IL-12 release type fusion proteins, where cleavable linker was introduced into elbow hinge region between VH and CH1 regions. Single-chain IL-12 was attached to C-terminal of Fc domain via cleavable linker. Digestion of cleavable linkers results in release of active IL-12. FIG. 3B, shows IL-12 fusion type fusion proteins, where cleavable linker was introduced into elbow hinge region between VH and CH1 regions. GS linker was inserted in hinge region and single-chain IL-12 was attached to C-terminal of Fc domain via GS linker. Digestion of cleavable linkers results in release of active IL-12 fused to Fc.

FIG. 4. Bivalent IL-12 release FP4, and Bivalent IL-12 fusion FP5 were subjected to the IL-12 luciferase assay. Both variants showed lower IL-12 bioactivity than hIL-12_His tag in the absence of MT-SP1, and the IL-12 bioactivity was restored to the same level as hIL-12_His tag upon MT-SP1 treatment

FIG. 5. Different formats of activated IL-12 fusion protein upon protease cleavage. (A) In release format, freely dissociated IL-12 molecule is the representative activated molecule, i.e. recombinant IL-12. (B) In the fusion format, KLH Bivalent fusion FP6 is the representative activated molecule.

FIG. 6. IL-12 concentrations in tumour lysate and tumour interstitial fluid of recombinant IL-12 or KLH-Bivalent IL12 fusion FP6 in human T cell injected LS1034 tumour bearing mice model after intra-tumour injection, repeated for a total of six times. Tumour retention levels comparing the activated forms of release and fusion format shows higher retention concentrations for the fusion format in both tumour lysate and interstitial fluid than the release format.

FIG. 7. Time course of plasma KLH-Bivalent IL-12 fusion FP6 concentration after intravenous administration in cynomolgus monkey. KLH-Bivalent IL-12 fusion FP6 was eliminated rapidly and the clearance was 1975 mL/day/kg, which is approximately 13-times faster than recombinant IL-12 clearance reported in literature which is 6.23 mL/hour/kg (150 mL/day/kg) (Pharmacology 2010; 85:319-327).

FIG. 8. (A) Time course analysis of KLH bivalent fusion FP7 showed a clearance of 335 ml/day/kg in SCID mice. (B) Time course analysis of inactive and active form of the IL-12 fusion protein. Clearance levels between inactive and active form of the IL-12 fusion protein was similar.

FIG. 9. (A) As activated and inactivated IL-12 fusion proteins showed similar clearance in FIG. 8, it is possible that this phenomenon was observed potentially as the VH domain, VL domain and IL-12 moiety may exhibit avidity and did not fully dissociate after protease cleavage. (B) Activity of the inactivated and activated form of the IL-12 fusion protein shows that the activated form remains capable of binding to IL-12 receptor after protease digestion and activates IL-12 signalling to the same extent as recombinant IL-12 independent of the average clearance observed in (A).

FIG. 10A. Schematic representation of Biacore assay to evaluate percentage dissociation of VH from the fusion protein. Representation of assay conducted for anti-IL-12 antibodies that bind IL-12.

FIG. 10B. Schematic representation of Biacore assay to evaluate percentage dissociation of VH from the fusion protein. Representation of assay conducted for bivalent IL-12 fusion protein.

FIG. 11A. Screening of amino acid modifications at the VH/VL interface to promote VH dissociation from anti-IL-12 antibody. Evaluation of single amino acid modifications and the percentage of VH dissociation.

FIG. 11B. Screening of amino acid modifications at the VH/VL interface to promote VH dissociation from anti-IL-12 antibody. Evaluation of combination of amino acid modifications and the percentage of VH dissociation.

FIG. 12A. Screening and evaluation of amino acid modifications at the VH/VL interface that promote VH dissociation from bivalent IL-12 fusion proteins.

FIG. 12B. Screening and evaluation of amino acid modifications at the VH/VL interface that promote VH dissociation from bivalent IL-12 fusion proteins.

FIG. 13. Time course of plasma concentrations of IL-12 fusion proteins with VH release modifications in SCID mice. Inclusion of VH/VL interface modification(s) have led to greater dissociation of IL-12 from the digested products of IL-12 fusion proteins and led to faster clearance compared to digested product of IL-12 fusion proteins without any modification(s) in the VH/VL interface.

FIG. 14A. Profile of CXCL10 fusion proteins. As inactive molecule, biological activity of CXCL10 should be inhibited and the CXCL10 fusion protein should have long systemic half-life. As an activated molecule, due to cleavage by disease specific proteases, CXCL10 biological activity is restored. In addition, the fusion protein should retain in high concentrations in disease tissue and should exhibit short systemic half-life.

FIG. 14B. Evaluation of amino acid modification to promote VH dissociation from the fusion protein.

FIG. 15. Screening and evaluation of amino acid modifications at the VH/VL interface that promote VH dissociation from bivalent IL-22 fusion proteins.

FIG. 16. Time course analysis of KLH bivalent fusion FP7 with and without MT-SP1 digestion. Digestion with MT-SP1 unexpectedly led to slower clearance of KLH bivalent fusion FP7. This could affect the profile of the activated IL12 molecule.

FIG. 17. SDS-PAGE analysis showing MT-SP1 mediated digestion using KLH bivalent fusion variants with protease resistant modifications in the heparin binding region of p40. Top panel shows undigested and digested samples with 1-hour incubation with MT-SP1. Bottom panel shows digested samples with 4-hour and 24-hour incubation with MT-SP1.

FIG. 18. SDS-PAGE analysis showing MT-SP1 mediated digestion using KLH bivalent fusion variants with protease resistant modifications in the heparin binding region of p40. Top panel shows undigested and digested samples with 1-hour incubation with MT-SP1. Bottom panel shows digested samples with 4-hour and 24-hour incubation with MT-SP1.

FIG. 19. IL-12 activity of protease resistant IL-12 variants was evaluated using Luciferase assay. All the protease resistant IL-12 variants indicated similar activity to hIL12_His tag regardless of protease treatment.

FIG. 20. Time course of plasma concentrations of protease resistant IL-12 variants as KLH-bivalent fusion in SCID mice. All the protease resistant variants demonstrated slower elimination that control (KLH-Bivalent IL12006v1).

FIG. 21. Bivalent IL-12 fusion proteins FP8, FP11 and FP12 were subjected to the IL-12 luciferase assay. All three fusion proteins showed lower IL-12 bioactivity than hIL-12_His tag in the absence of MT-SP1, and the IL-12 bioactivity was restored to the same level as hIL-12_His tag upon MT-SP1 treatment.

FIG. 22A. Schematic diagram of IL-22 release from the fusion protein “FP14” from which VH-IL-22 is released.

FIG. 22B. Evaluation of the activity of IL-22 fusion proteins with/without protease digestion. To assess if the modifications of the amino acids at the VH/VL interface promoted the release of IL-22, the IL-22 activity was assayed using the concentration of IL-10 which is secreted from cells in response to IL-22. A total of 3 assay plates were assayed, FIG. 22B corresponds to the evaluation results of plate 1. For each assay plate, IL-22 fusion protein without any modifications at the VH/VL interface, i.e. control fusion protein (“Ab4H/Ab4L FP14”) was included as a reference. To compare the IL-22 activity between IL-22 fusion proteins, interpolation of IL-10 response curve was set at concentration of 200 pg/mL. The activity window was calculated as the ratio of the concentration of each IL-22 fusion protein to induce 200 pg/mL IL-10 with and without uPA protease.

FIG. 22C. Evaluation of the activity of IL-22 fusion proteins with/without protease digestion. To assess if the modifications of the amino acids at the VH/VL interface promoted the release of IL-22, the IL-22 activity was assayed using the concentration of IL-10 which is secreted from cells in response to IL-22. A total of 3 assay plates were assayed, FIG. 22C corresponds to the evaluation results of plate 1. For each assay plate, IL-22 fusion protein without any modifications at the VH/VL interface, i.e. control fusion protein (“Ab4H/Ab4L FP14”) was included as a reference. To compare the IL-22 activity between IL-22 fusion proteins, interpolation of IL-10 response curve was set at concentration of 200 pg/mL. The activity window was calculated as the ratio of the concentration of each IL-22 fusion protein to induce 200 pg/mL IL-10 with and without uPA protease.

FIG. 22D. Evaluation of the activity of IL-22 fusion proteins with/without protease digestion. To assess if the modifications of the amino acids at the VH/VL interface promoted the release of IL-22, the IL-22 activity was assayed using the concentration of IL-10 which is secreted from cells in response to IL-22. A total of 3 assay plates were assayed, FIG. 22D corresponds to the evaluation results of plate 2. For each assay plate, IL-22 fusion protein without any modifications at the VH/VL interface, i.e. control fusion protein (“Ab4H/Ab4L FP14”) was included as a reference. To compare the IL-22 activity between IL-22 fusion proteins, interpolation of IL-10 response curve was set at concentration of 200 pg/mL. The activity window was calculated as the ratio of the concentration of each IL-22 fusion protein to induce 200 pg/mL IL-10 with and without uPA protease.

FIG. 22E. Evaluation of the activity of IL-22 fusion proteins with/without protease digestion. To assess if the modifications of the amino acids at the VH/VL interface promoted the release of IL-22, the IL-22 activity was assayed using the concentration of IL-10 which is secreted from cells in response to IL-22. A total of 3 assay plates were assayed, FIG. 22E corresponds to the evaluation results of plate 2. For each assay plate, IL-22 fusion protein without any modifications at the VH/VL interface, i.e. control fusion protein (“Ab4H/Ab4L FP14”) was included as a reference. To compare the IL-22 activity between IL-22 fusion proteins, interpolation of IL-10 response curve was set at concentration of 200 pg/mL. The activity window was calculated as the ratio of the concentration of each IL-22 fusion protein to induce 200 pg/mL IL-10 with and without uPA protease.

FIG. 22F. Evaluation of the activity of IL-22 fusion proteins with/without protease digestion. To assess if the modifications of the amino acids at the VH/VL interface promoted the release of IL-22, the IL-22 activity was assayed using the concentration of IL-10 which is secreted from cells in response to IL-22. A total of 3 assay plates were assayed, FIG. 22F correspond to the evaluation results of plate 3. For each assay plate, IL-22 fusion protein without any modifications at the VH/VL interface, i.e. control fusion protein (“Ab4H/Ab4L FP14”) was included as a reference. To compare the IL-22 activity between IL-22 fusion proteins, interpolation of IL-10 response curve was set at concentration of 200 pg/mL. The activity window was calculated as the ratio of the concentration of each IL-22 fusion protein to induce 200 pg/mL IL-10 with and without uPA protease.

FIG. 23A. Schematic diagram of IL-22 release from the fusion protein “FP15” from which VL-IL-22 is released.

FIG. 23B. Evaluation of the activity of IL-22 fusion proteins with/without protease digestion. To assess if the modifications of the amino acids at the VH/VL interface promoted the release of IL-22, the IL-22 activity was assayed using the concentration of IL-10 which is secreted from cells in response to IL-22. A total of 2 assay plates were assayed, FIG. 23B corresponds to the evaluation results of plate 1. For each assay plate, IL-22 fusion protein without any modifications at the VH/VL interface, i.e. control fusion protein (“Ab5H/Ab5L FP15”) was included as a reference. To compare the IL-22 activity between IL-22 fusion proteins, interpolation of IL-10 response curve was set at concentration of 200 pg/mL. The activity window was calculated as the ratio of the concentration of each IL-22 fusion protein to induce 200 pg/mL IL-10 with and without uPA protease.

FIG. 23C. Evaluation of the activity of IL-22 fusion proteins with/without protease digestion. To assess if the modifications of the amino acids at the VH/VL interface promoted the release of IL-22, the IL-22 activity was assayed using the concentration of IL-10 which is secreted from cells in response to IL-22. A total of 2 assay plates were assayed, FIG. 23C corresponds to the evaluation results of plate 1. For each assay plate, IL-22 fusion protein without any modifications at the VH/VL interface, i.e. control fusion protein (“Ab5H/Ab5L FP15”) was included as a reference. To compare the IL-22 activity between IL-22 fusion proteins, interpolation of IL-10 response curve was set at concentration of 200 pg/mL. The activity window was calculated as the ratio of the concentration of each IL-22 fusion protein to induce 200 pg/mL IL-10 with and without uPA protease.

FIG. 23D. Evaluation of the activity of IL-22 fusion proteins with/without protease digestion. To assess if the modifications of the amino acids at the VH/VL interface promoted the release of IL-22, the IL-22 activity was assayed using the concentration of IL-10 which is secreted from cells in response to IL-22. A total of 2 assay plates were assayed, FIG. 23D corresponds to the evaluation results of plate 2. For each assay plate, IL-22 fusion protein without any modifications at the VH/VL interface, i.e. control fusion protein (“Ab5H/Ab5L FP15”) was included as a reference. To compare the IL-22 activity between IL-22 fusion proteins, interpolation of IL-10 response curve was set at concentration of 200 pg/mL. The activity window was calculated as the ratio of the concentration of each IL-22 fusion protein to induce 200 pg/mL IL-10 with and without uPA protease.

FIG. 23E. Evaluation of the activity of IL-22 fusion proteins with/without protease digestion. To assess if the modifications of the amino acids at the VH/VL interface promoted the release of IL-22, the IL-22 activity was assayed using the concentration of IL-10 which is secreted from cells in response to IL-22. A total of 2 assay plates were assayed, FIG. 23E corresponds to the evaluation results of plate 2. For each assay plate, IL-22 fusion protein without any modifications at the VH/VL interface, i.e. control fusion protein (“Ab5H/Ab5L FP15”) was included as a reference. To compare the IL-22 activity between IL-22 fusion proteins, interpolation of IL-10 response curve was set at concentration of 200 pg/mL. The activity window was calculated as the ratio of the concentration of each IL-22 fusion protein to induce 200 pg/mL IL-10 with and without uPA protease.

FIG. 24A. Schematic diagram of IL-22 release from the fusion protein “FP16” from which VH is released.

FIG. 24B. Evaluation of the activity of IL-22 fusion proteins with/without protease digestion. To assess if the modifications of the amino acids at the VH/VL interface promoted the release of IL-22, the IL-22 activity was assayed using the concentration of IL-10 which is secreted from cells in response to IL-22. A total of 2 assay plates were assayed, FIG. 24B corresponds to the evaluation results of plate 1. For each assay plate, IL-22 fusion protein without any modifications at the VH/VL interface, i.e. control fusion protein (“Ab5H/Ab5L FP16”) was included as a reference. To compare the IL-22 activity between IL-22 fusion proteins, interpolation of IL-10 response curve was set at concentration of 300 pg/mL. The activity window was calculated as the ratio of the concentration of each IL-22 fusion protein to induce 300 pg/mL IL-10 with and without uPA protease.

FIG. 24C. Evaluation of the activity of IL-22 fusion proteins with/without protease digestion. To assess if the modifications of the amino acids at the VH/VL interface promoted the release of IL-22, the IL-22 activity was assayed using the concentration of IL-10 which is secreted from cells in response to IL-22. A total of 2 assay plates were assayed, FIG. 24C corresponds to the evaluation results of plate 1. For each assay plate, IL-22 fusion protein without any modifications at the VH/VL interface, i.e. control fusion protein (“Ab5H/Ab5L FP16”) was included as a reference. To compare the IL-22 activity between IL-22 fusion proteins, interpolation of IL-10 response curve was set at concentration of 300 pg/mL. The activity window was calculated as the ratio of the concentration of each IL-22 fusion protein to induce 300 pg/mL IL-10 with and without uPA protease.

FIG. 24D. Evaluation of the activity of IL-22 fusion proteins with/without protease digestion. To assess if the modifications of the amino acids at the VH/VL interface promoted the release of IL-22, the IL-22 activity was assayed using the concentration of IL-10 which is secreted from cells in response to IL-22. A total of 2 assay plates were assayed, FIG. 24D corresponds to the evaluation results of plate 2. For each assay plate, IL-22 fusion protein without any modifications at the VH/VL interface, i.e. control fusion protein (“Ab5H/Ab5L FP16”) was included as a reference. To compare the IL-22 activity between IL-22 fusion proteins, interpolation of IL-10 response curve was set at concentration of 300 pg/mL. The activity window was calculated as the ratio of the concentration of each IL-22 fusion protein to induce 300 pg/mL IL-10 with and without uPA protease.

FIG. 24E. Evaluation of the activity of IL-22 fusion proteins with/without protease digestion. To assess if the modifications of the amino acids at the VH/VL interface promoted the release of IL-22, the IL-22 activity was assayed using the concentration of IL-10 which is secreted from cells in response to IL-22. A total of 2 assay plates were assayed, FIG. 24E corresponds to the evaluation results of plate 2. For each assay plate, IL-22 fusion protein without any modifications at the VH/VL interface, i.e. control fusion protein (“Ab5H/Ab5L FP16”) was included as a reference. To compare the IL-22 activity between IL-22 fusion proteins, interpolation of IL-10 response curve was set at concentration of 300 pg/mL. The activity window was calculated as the ratio of the concentration of each IL-22 fusion protein to induce 300 pg/mL IL-10 with and without uPA protease.

FIG. 25A. Evaluation of the activity of IL-22 fusion proteins with/without protease digestion and the activity of recombinant IL-22. IL-22 activity of IL-22 fusion protein without mutation at the VH/VL interface and a selected IL-22 fusion protein variant for FP14 was evaluated in the presence or absence of uPA protease, with recombinant IL-22 as a reference control. To compare the IL-22 activity, interpolation of IL-10 response curve was set at concentration of 250 pg/mL for FP14. The activity window for each fusion protein was calculated as the ratio of the concentration of the IL-22 fusion protein to induce the indicated amount of IL-10 with and without uPA protease. All three selected fusion proteins variants showed lower IL-22 bioactivity that recombinant human IL-22 in the absence of uPA, and the IL-22 bioactivity was restored to the same level as recombinant human IL-22 in the presence of uPA.

FIG. 25B. Evaluation of the activity of IL-22 fusion proteins with/without protease digestion and the activity of recombinant IL-22. IL-22 activity of IL-22 fusion protein without mutation at the VH/VL interface and a selected IL-22 fusion protein variant for FP15 was evaluated in the presence or absence of uPA protease, with recombinant IL-22 as a reference control. To compare the IL-22 activity, interpolation of IL-10 response curve was set at concentration of 400 pg/mL for FP15. The activity window for each fusion protein was calculated as the ratio of the concentration of the IL-22 fusion protein to induce the indicated amount of IL-10 with and without uPA protease. All three selected fusion proteins variants showed lower IL-22 bioactivity that recombinant human IL-22 in the absence of uPA, and the IL-22 bioactivity was restored to the same level as recombinant human IL-22 in the presence of uPA.

FIG. 25C. Evaluation of the activity of IL-22 fusion proteins with/without protease digestion and the activity of recombinant IL-22. IL-22 activity of IL-22 fusion protein without mutation at the VH/VL interface and a selected IL-22 fusion protein variant for FP16 was evaluated in the presence or absence of uPA protease, with recombinant IL-22 as a reference control. To compare the IL-22 activity, interpolation of IL-10 response curve was set at concentration of 400 pg/mL for FP16. The activity window for each fusion protein was calculated as the ratio of the concentration of the IL-22 fusion protein to induce the indicated amount of IL-10 with and without uPA protease. All three selected fusion proteins variants showed lower IL-22 bioactivity that recombinant human IL-22 in the absence of uPA, and the IL-22 bioactivity was restored to the same level as recombinant human IL-22 in the presence of uPA.

### DESCRIPTION OF EMBODIMENTS

### General Techniques

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds 1987, and periodic updates); PCR: The Polymerase Chain Reaction, (Mullis et al., ed., 1994); A Practical Guide to Molecular Cloning (Perbal Bernard V., 1988); Phage Display: A Laboratory Manual (Barbas et al., 2001).

The definitions and detailed description below are provided to facilitate understanding of the present disclosure illustrated herein. All references mentioned herein are specifically incorporated by reference.

### I. Definitions

### Protein/Polypeptide

As used herein, term “polypeptide” refers to a molecule composed of monomers (amino acids) linearly linked by amide bonds (also known as peptide bonds). The term “polypeptide” usually refers to a peptide having a length on the order of 4 amino acids or longer, and does not refer to a specific length of the product. As used herein, the term also includes fragments of polypeptides. Thus, peptides, dipeptides, tripeptides, oligopeptides, “protein,” “amino acid chain,” or any other term used to refer to a chain of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. A polypeptide as described herein may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations and are referred to as unfolded.

### Amino Acids

Herein, amino acids are described by one-letter code or three-letter code, or both, as represented by, for example, Ala/A, Leu/L, Arg/R, Lys/K, Asn/N, Met/M, Asp/D, Phe/F, Cys/C, Pro/P, Gln/Q, Ser/S, Glu/E, Thr/T, Gly/G, Trp/W, His/H, Tyr/Y, Ile/I, or Val/V. For expressing an amino acid located at a particular position, an expression using a number representing the particular position in combination with the one-letter code or the three-letter code of the amino acid can be appropriately used. For example, an amino acid 37V, which is an amino acid contained in a variable region of an antibody, represents Val located at position 37 defined by the Kabat numbering.

### Amino Acid Modification

The terms “amino acid modification”, “amino acid alteration” or “amino acid mutation” as used interchangeably herein, refer to the alteration of an amino acid in the amino acid sequence of a protein or polypeptide by a method known in the art that can be appropriately adopted such as site-directed mutagenesis (Kunkel et al. (Proc. Natl. Acad. Sci. USA (1985) 82, 488-492)) or overlap extension PCR. Several methods known in the art can also be adopted as alteration methods for substituting an amino acid by an amino acid other than a natural amino acid (Annu. Rev. Biophys. Biomol. Struct. (2006) 35, 225-249; and Proc. Natl. Acad. Sci. U.S.A. (2003) 100 (11), 6353-6357). For example, a tRNA-containing cell-free translation system (Clover Direct (Protein Express)) having a non-natural amino acid bound with amber suppressor tRNA complementary to UAG codon (amber codon), which is a stop codon, is also preferably used. In the present specification, examples of an amino acid modification at a specified position include the substitution or deletion of the specified residue, or the insertion of at least one amino acid residue adjacent the specified residue or any combination of substitution, deletion and insertion thereof. Insertion “adjacent” to a specified residue means insertion within one to two residues thereof. The insertion may be N-terminal or C-terminal to the specified residue. The preferred amino acid modification herein is a substitution.

### Substitution

An “amino acid substitution” refers to the replacement of at least one existing amino acid residue in a predetermined amino acid sequence with another different “replacement” amino acid residue. The replacement residue or residues may be “naturally occurring amino acid residues” (i.e. encoded by the genetic code) and selected from the group consisting of: alanine (Ala); arginine (Arg); asparagine (Asn); aspartic acid (Asp); cysteine (Cys); glutamine (Gln); glutamic acid (Glu); glycine (Gly); histidine (His); isoleucine (Ile): leucine (Leu); lysine (Lys); methionine (Met); phenylalanine (Phe); proline (Pro); serine (Ser); threonine (Thr); tryptophan (Trp); tyrosine (Tyr); and valine (Val). Preferably, the replacement residue is not cysteine. Substitution with one or more non-naturally occurring amino acid residues is also encompassed by the definition of an amino acid substitution herein. A “non-naturally occurring amino acid residue” refers to a residue, other than those naturally occurring amino acid residues listed above, which is able to covalently bind adjacent amino acid residues(s) in a polypeptide chain. Examples of non-naturally occurring amino acid residues include norleucine, ornithine, norvaline, homoserine and other amino acid residue analogues such as those described in Ellman et al. Meth. Enzym. 202:301-336 (1991). To generate such non-naturally occurring amino acid residues, the procedures of Noren et al. Science 244:182 (1989) and Ellman et al., supra, can be used. Briefly, these procedures involve chemically activating a suppressor tRNA with a non-naturally occurring amino acid residue followed by in vitro transcription and translation of the RNA.

### Insertion

An “amino acid insertion” refers to the incorporation of at least one amino acid into a predetermined amino acid sequence. While the insertion will usually consist of the insertion of one or two amino acid residues, the present application contemplates larger “peptide insertions”, e.g. insertion of about three to about five or even up to about ten amino acid residues. The inserted residue(s) may be naturally occurring or non-naturally occurring as disclosed above.

### Deletion

An “amino acid deletion” refers to the removal of at least one amino acid residue from a predetermined amino acid sequence.

The term “and/or” as used herein when referring to a site of amino acid alteration includes every combination appropriately represented by “and/or”. Specifically, for example, the phrase “amino acids at positions 37, 45, and/or 47 are substituted” includes the following variations of amino acid alteration: (a) position 37, (b) position 45, (c) position 47, (d) positions 37 and 45, (e) positions 37 and 47, (f) positions 45 and 47, and (g) positions 37, 45 and 47.

In the present specification, expression in which the one-letter codes or three-letter-codes of amino acids before and after alteration are used previous and next to a number representing a particular position can be appropriately used for representing amino acid alteration. For example, an alteration F37V or Phe37Val used for substituting an amino acid contained in an antibody variable region represents the substitution of Phe at position 37 defined by the Kabat numbering by Val. Specifically, the number represents an amino acid position defined by the Kabat numbering; the one-letter code or three-letter code of the amino acid previous to the number represents the amino acid before the substitution; and the one-letter code or three-letter code of the amino acid next to the number represents the amino acid after the substitution. Likewise, an alteration P238A or Pro238Ala used for substituting an amino acid in a Fc region contained in an antibody constant region represents the substitution of Pro at position 238 defined by the EU numbering by Ala. Specifically, the number represents an amino acid position defined by the EU numbering; the one-letter code or three-letter code of the amino acid previous to the number represents the amino acid before the substitution; and the one-letter code or three-letter code of the amino acid next to the number represents the amino acid after the substitution.

### Percent (%) Amino Acid Identity

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

### Ligand-Binding Moiety/Molecule

In some embodiments, the fusion protein is a polypeptide comprising a ligand-binding moiety or ligand-binding molecule further comprising a ligand-binding domain. The terms “ligand-binding moiety” or “ligand-binding molecule” as used herein, refer to a moiety or molecule that is capable of binding to a ligand, and particularly refers to a moiety or molecule that binds to a ligand when the moiety or molecule is in the uncleaved state. In this context, the “binding” usually refers to binding through interaction based mainly on a noncovalent bond such as electrostatic force, van der Waals' force, or a hydrogen bond. Preferred examples of the binding mode of the ligand-binding moiety or molecule include, but are not limited to, antigen-antibody reaction through which an antigen-binding domain, an antigen-binding molecule, an antibody, an antibody fragment, or the like binds to the antigen. In certain embodiments, the ligand-binding moiety or molecule includes, but is not limited to, antibody fragments, antibodies, and molecules formed from antibody fragments (e.g. diabodies, chimeric antigen receptors (CARs)), including multispecific binding molecules (e.g. bispecific diabodies and bispecific antibodies).

### Ligand-Binding Domain

The term “ligand-binding domain” as used herein, refers to a portion of a ligand-binding moiety or molecule which binds only to a portion of a ligand (epitope) when the ligand-binding moiety/molecule binds to the ligand. In the present invention, the ligand-binding domain is limited only by the fact that the domain binds to a ligand when the ligand-binding moiety/molecule is in the uncleaved state, and may have any structure as long as the domain can bind to a ligand of interest when the ligand-binding moiety/molecule is in the uncleaved state. Examples of the ligand-binding domain include, but are not limited to, an antigen-binding domain, an antibody heavy chain variable region (VH), an antibody light chain variable region (VL), an antibody Fv region, a single-domain antibody (sdAb), a scaffold peptide, a peptide aptamer (Reverdatto S. et al., Curr Top Med Chem. 2015; 15(12): 1082-1101), IL-12 receptor, a module called A domain of approximately 35 amino acids contained in an in vivo cell membrane protein avimer (WO2004/044011 and WO2005/040229), adnectin containing a 10Fn3 domain serving as a protein binding domain derived from a glycoprotein fibronectin expressed on cell membranes (WO2002/032925), Affibody containing an IgG binding domain scaffold constituting a three-helix bundle composed of 58 amino acids of protein A (WO1995/001937), DARPins (designed ankyrin repeat proteins) which are molecular surface-exposed regions of ankyrin repeats (AR) each having a 33-amino acid residue structure folded into a subunit of a turn, two antiparallel helices, and a loop (WO2002/020565), anticalin having four loop regions connecting eight antiparallel strands bent toward the central axis in one end of a barrel structure highly conserved in lipocalin molecules such as neutrophil gelatinase-associated lipocalin (NGAL) (WO2003/029462), and a depressed region in the internal parallel sheet structure of a horseshoe-shaped fold composed of repeated leucine-rich-repeat (LRR) modules of an immunoglobulin structure-free variable lymphocyte receptor (VLR) as seen in the acquired immune systems of jawless vertebrates such as lamprey or hagfish (WO2008/016854).

### Antigen-Binding Domain

The term “antigen-binding domain” as used herein, refers to a region that specifically binds to or partially complements an antigen. As used herein, an antigen binding molecule comprises an antigen-binding domain. If the molecular weight of the antigen is large, the antigen-binding domain can only bind to a specific part of the antigen. The specific part is called an epitope. In one embodiment, the antigen-binding domain comprises an antibody fragment that binds to a particular antigen. The antigen-binding domain may be provided by one or more antibody variable domains. In one non-limiting embodiment, the antigen-binding domain comprises an antibody light chain variable region (VL) and an antibody heavy chain variable region (VH). Examples of such antigen-binding domains include “scFv (single-chain Fv)”, “single-chain antibody (single-chain antibody)”, “Fv”, “scFv2 (single-chain Fv 2)”, “Fab” or “F(ab′)2”, and the like. In another embodiment, the antigen-binding domain comprises a non-antibody protein or a fragment thereof that binds to a particular antigen. In certain embodiments, the antigen-binding domain comprises a hinge region. In some embodiments, the antigen is a ligand. As used herein, where the antigen is a ligand, the terms “antigen-binding domain” and “ligand-binding domain” may be used interchangeably to refer to the region that specifically or partially binds the ligand as the antigen.

As used herein, the term “binding to the same epitope” means that the epitopes to which two antigen binding domains bind overlap at least partially. The degree of overlap is not limited, but is at least 10% or more, preferably 20% or more, 30% or more, 40% or more, 50% or more, 60% or more, 70% or more, 80% or more, and particularly preferably 90% or more. Most preferably 100% overlap.

In one embodiment, the fusion protein of the present invention comprises an antigen binding-domain that may bind an antigen, e.g. Interleukin-12 (IL-12) or IL-22. The terms “a fusion protein that binds IL-12”, “a polypeptide that binds IL-12”, or “an antibody that binds to IL-12” or “anti-IL-12” antibody refer to measurable and reproducible interactions between the protein or antibody with IL-12, which is determinative of the presence of its antigen, e.g. IL-12 in the presence of a heterogenous population of molecules including biological molecules. The same applies to IL-22, etc. The fusion protein or antibody that binds to its antigen with greater affinity, avidity, more readily, and/or with greater duration than it binds to other antigens. In one embodiment, the extent of binding of the fusion protein or the antibody to an unrelated antigen is less than about 10% of the binding of the fusion protein or the antibody to the antigen as measured, e.g., by a radioimmunoassay (RIA). In certain embodiments, a fusion protein or an antibody that specifically binds to an antigen/target has a dissociation constant (Kd) of 1 micromolar (micro M) or less, 100 nM or less, 10 nM or less, 1 nM or less, 0.1 nM or less, 0.01 nM or less, or 0.001 nM or less (e.g. 10⁻⁸ M or less, e.g. from 10⁻⁸ M to 10⁻¹³ M, e.g., from 10⁻⁹ M to 10⁻¹³ M). In certain embodiments, a fusion protein or an antibody specifically binds to an epitope on a protein that is conserved among the protein from different species. In another embodiment, specific binding can include, but does not require exclusive binding.

### Ligand/Antigen

As used herein, the term “ligand” (which may alternatively be called “ligand moiety”) and “antigen” may be used interchangeably and is limited only by containing an epitope to which the ligand-binding domain or antigen-binding domain binds. The term “ligand” and “antigen” refer to all molecules that can be specifically bound by the ligand-binding domain or antigen binding domain. Preferred examples of the ligand/antigen include, but are not limited to, animal- or human-derived peptides, polypeptides, and proteins. Preferred examples of the ligand/antigen for use in the treatment of a disease caused by a target tissue include, but are not limited to molecules expressed on the surface of target cells (e.g., cancer cells and inflammatory cells), molecules expressed on the surface of other cells in tissues containing target cells, molecules expressed on the surface of cells having an immunological role against target cells and tissues containing target cells, macromolecules present in the stroma of tissues containing target cells, soluble molecules such as cytokines, chemokines, polypeptide hormones, growth factors, apoptosis inducing factors, PAMPs, DAMPs, nucleic acids, and fragments thereof, or other molecules involved in immunomodulatory and inflammatory processes. Examples of the ligand or antigen includes an interleukin, an interferon, a hematopoietic factor, a member of the TNF superfamily, a chemokine, a cell growth factor, a member of the TGF-beta family, a myokine, an adipokine, or a neurotrophic factor. More specifically, examples include CXCL9, CXCL10, CXCL11, IL-2, IL-7, IL-12, IL-15, IL-18, IL-21, IL-22, IFN-alpha, IFN-beta, IFN-gamma, MIG, I-TAC, RANTES, MIP-1a, MIP-1b, IL-1R1 (Interleukin-1 receptor, type I), IL-1R2 (Interleukin-1 receptor, type II), IL-1RAcP (Interleukin-1 receptor accessory protein), or IL-1Ra (Protein Accession No. NP_776214, mRNA Accession No. NM_173842.2)

### Specificity

As used herein, the term “specificity” refers to a property by which one of specifically binding molecules does not substantially bind to a molecule other than its one or more binding partner molecules. This term is also used when the antigen-binding domain has specificity for an epitope contained in a particular antigen. The term is also used when the antigen-binding domain has specificity for a particular epitope among a plurality of epitopes contained in an antigen. In this context, the term “not substantially bind” is determined according to the method described in the section about binding activity and means that the binding activity of a specific binding molecule for a molecule other than the binding partner(s) is 80% or less, usually 50% or less, preferably 30% or less, particularly preferably 15% or less, of its binding activity for the binding partner molecule(s).

### Affinity

The term “affinity” as used herein, refers to the strength of the sum total of noncovalent interactions between a single binding site of a molecule (e.g., a ligand-binding molecule, a ligand, an antigen-binding molecule or an antibody) and its binding partner (e.g., a ligand, a ligand receptor, or an antigen). Unless indicated otherwise, as used herein, “binding affinity” refers to intrinsic binding affinity which reflects a 1:1 interaction between members of a binding pair (e.g., a ligand-binding molecule and a ligand, a ligand and a ligand receptor, an antigen-binding molecule and an antigen or an antibody and an antigen). The affinity of a molecule X for its partner Y can generally be represented by the dissociation constant (Kd), which is the ratio of dissociation and association rate constants (Koff and Kon, respectively). Affinity can be measured by common methods known in the art, including those described herein. Specific illustrative and exemplary embodiments for measuring binding affinity are described in the following.

### Antibody and Antibody Fragments

In one embodiment, the ligand-binding moiety of the presently claimed fusion protein comprises an antibody. As used herein, the term “antibody” is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

The term an “antibody fragment” as used herein, refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); and multispecific antibodies formed from antibody fragments.

### Full-Length/Native Antibody

The terms “full-length antibody”, “intact antibody”, and “whole antibody” are used herein interchangeably to refer to an antibody having a structure substantially similar to a native antibody structure or having heavy chains that contain a Fc region as defined herein.

The term “native antibodies” as used herein, refer to naturally occurring immunoglobulin molecules with varying structures. For example, native IgG antibodies are heterotetrameric glycoproteins of about 150,000 daltons, composed of two identical light chains and two identical heavy chains that are disulphide bonded. From N- to C-terminus, each heavy chain has a variable region (VH), also called a variable heavy domain or a heavy chain variable domain (VH), or an antibody heavy chain variable domain (VH), or antibody heavy chain variable region (VH), followed by three constant domains (CH1, CH2, and CH3). Similarly, from N- to C-terminus, each light chain has a variable region (VL), also called a variable light domain (VL) or a light chain variable domain (VL), or an antibody light chain variable domain (VH), or antibody light chain variable region (VH), followed by a constant light (CL) domain. The light chain of an antibody may be assigned to one of two types, called kappa and lambda, based on the amino acid sequence of its constant domain. As used herein, the terms “CL”, or “CL region”, or “CL domain” are used interchangeably, and the same is applicable to the other domains, “VH”, “VL”, “CH1”, “CH2” and “CH3”, when each of these terms are paired with “region” or “domain” in reference.

### Monoclonal Antibody

The term “monoclonal antibody” as used herein, refers to an antibody obtained from a population of substantially homogeneous antibodies, i.e., the individual antibodies composing the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

### Chimeric, Humanised and Human Antibody

The term “chimeric” antibody as used herein, refers to an antibody in which a portion of the heavy and/or light chain is derived from a particular source or species, while the remainder of the heavy and/or light chain is derived from a different source or species.

A “humanised” (“humanized”) antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

### Human Consensus Framework

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one embodiment, for the VL, the subgroup is subgroup kappa I as in Kabat et al., supra. In one embodiment, for the VH, the subgroup is subgroup III as in Kabat et al., supra.

### Acceptor Human Framework

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

### Affinity Matured Antibody

The term “affinity matured” antibody as used herein, refers to an antibody with one or more alterations in one or more hypervariable regions (HVRs), compared to a parent antibody which does not possess such alterations, such alterations resulting in an improvement in the affinity of the antibody for antigen.

### Antibody Classes

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called alpha, delta, epsilon, gamma, and mu, respectively.

### IgG Antibody-Like Polypeptide

The term “IgG antibody-like polypeptide” or “IgG antibody-like molecule” used in the present specification is used to define a polypeptide having moieties substantially similar in structure to constant domains or constant regions as in an IgG antibody, and moieties substantially similar in structure to variable domains or variable regions as in the IgG antibody, and having conformation substantially similar to that of the IgG antibody. In the IgG antibody-like molecule, the domain similar to antibody CH1 and the domain similar to CL may be used interchangeably; that is, as long as interaction similar to the interaction between CH1 and CL of an IgG antibody is present between the domains, the domains linked to the portion similar to the antibody hinge region may be an antibody CH1 domain or an antibody CL domain. However, in the present specification, the “IgG antibody-like molecule” may or may not exert antigen-binding activity while retaining the structures similar to those of the IgG antibody. As used herein, the term “full-length IgG antibody comprising a protease cleavage site” or wherein a full-length IgG antibody that comprises a protease cleavage site is referred in the present specification, such terms or phrases are used interchangeably to refer to the abovementioned “IgG antibody-like polypeptide” or “IgG antibody-like molecule” as long as it achieves the purpose for the proper function of the present fusion protein.

### Substantially Similar

The term “substantially similar” or “substantially the same,” as used herein, refers to a sufficiently high degree of similarity between two numeric values (for example, one associated with an antibody of the invention and the other associated with a reference/comparator antibody), such that one of skill in the art would consider the difference between the two values to be of little or no biological and/or statistical significance within the context of the biological characteristic measured by said values (e.g., Kd values).

### Constant and Fc Regions

The terms “constant region” or “constant domain” as used herein refer to a region or a domain other than variable regions in an antibody. For example, an IgG antibody is a heterotetrameric glycoprotein of approximately 150,000 Da constituted by two identical light chains and two identical heavy chains connected through disulphide bonds. Each heavy chain has a variable region (VH) also called variable heavy chain domain or heavy chain variable domain, followed by a heavy chain constant region (CH) containing a CH1 domain, a hinge region, a CH2 domain, and a CH3 domain, from the N terminus toward the C terminus. Likewise, each light chain has a variable region (VL) also called variable light chain domain or light chain variable domain, followed by a constant light chain (CL) domain, from the N terminus toward the C terminus. The light chains of natural antibodies may be attributed to one of two types called kappa and lambda on the basis of the amino acid sequences of their constant domains. As used herein, the terms “CH1”, “CH1 domain”, “CH1 region” are used interchangeably. As used herein, the terms “CL”, “CL domain”, “CL region” are used interchangeably.

The term “Fc region” as used herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) or glycine-lysine (residues 446-447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, M D, 1991, unless otherwise specified.

### Variant Fc Region

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

### Variant Constant Region

A “variant constant region” comprises an amino acid sequence which differs from that of a native sequence constant region by virtue of at least one amino acid modification, preferably one or more amino acid substitution(s). Preferably, the variant constant region has at least one amino acid substitution compared to a native sequence constant region or to the constant region of a parent polypeptide, e.g. from about one to about ten amino acid substitutions, and preferably from about one to about five amino acid substitutions in a native sequence constant region or in the constant region of the parent polypeptide. The variant constant region herein will preferably possess at least about 80% homology with a native sequence constant region and/or with a constant region of a parent polypeptide, and most preferably at least about 90% homology therewith, more preferably at least about 95% homology therewith.

### Fc Receptor

The term “Fc receptor” or “FcR” refers to a receptor that binds to the Fc region of an antibody. In some embodiments, an FcR is a native human FcR. In some embodiments, an FcR is one which binds an IgG antibody (a gamma receptor) and includes receptors of the Fc gamma RI, Fc gamma RII, and Fc gamma RIII subclasses, including allelic variants and alternatively spliced forms of those receptors. Fc gamma RII receptors include Fc gamma RIIA (an “activating receptor”) and Fc gamma RIIB (an “inhibiting receptor”), which have similar amino acid sequences that differ primarily in the cytoplasmic domains thereof. Activating receptor Fc gamma RIIA contains an immunoreceptor tyrosine-based activation motif (ITAM) in its cytoplasmic domain. Inhibiting receptor Fc gamma RIIB contains an immunoreceptor tyrosine-based inhibition motif (ITIM) in its cytoplasmic domain. (see, e.g., Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein.

The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

Binding to human FcRn in vivo and plasma half-life of human FcRn high affinity binding polypeptides can be assayed, e.g., in transgenic mice or transfected human cell lines expressing human FcRn, or in primates to which the polypeptides with a variant Fc region are administered. WO 2000/42072 (Presta) describes antibody variants with increased or decreased binding to FcRs. See also, e.g., Shields et al. J. Biol. Chem. 9(2):6591-6604 (2001).

### Fc Region-Comprising Antibody

The term “Fc region-comprising antibody” refers to an antibody that comprises an Fc region. The C-terminal lysine (residue 447 according to the EU numbering system) or C-terminal glycine-lysine (residues 446-447) of the Fc region may be removed, for example, during purification of the antibody or by recombinant engineering of the nucleic acid encoding the antibody. Accordingly, a composition comprising an antibody having an Fc region according to this invention can comprise an antibody with G446-K447, with G446 and without K447, with all G446-K447 removed, or a mixture of three types of antibodies described above.

### Functional Fc Region

A “functional Fc region” possesses an “effector function” of a native sequence Fc region. Exemplary “effector functions” include C1q binding; CDC; Fc receptor binding; ADCC; phagocytosis; down regulation of cell surface receptors (e.g. B cell receptor; BCR), etc. Such effector functions generally require the Fc region to be combined with a binding domain (e.g., an antibody variable domain) and can be assessed using various assays as disclosed, for example, in definitions herein.

### Human Effector Cells

“Human effector cells” refer to leukocytes that express one or more FcRs and perform effector functions. In certain embodiments, the cells express at least Fc gamma RIII and perform ADCC effector function(s). Examples of human leukocytes which mediate ADCC include peripheral blood mononuclear cells (PBMC), natural killer (NK) cells, monocytes, cytotoxic T cells, and neutrophils. The effector cells may be isolated from a native source, e.g., from blood.

### Antibody-Dependent Cell-Mediated Cytotoxicity

“Antibody-dependent cell-mediated cytotoxicity” or “ADCC” refers to a form of cytotoxicity in which secreted Ig bound onto Fc receptors (FcRs) present on certain cytotoxic cells (e.g. NK cells, neutrophils, and macrophages) enable these cytotoxic effector cells to bind specifically to an antigen-bearing target cell and subsequently kill the target cell with cytotoxins. The primary cells for mediating ADCC, NK cells, express Fc gamma RIII only, whereas monocytes express Fc gamma RI, Fc gamma RII, and Fc gamma RIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 or U.S. Pat. No. 6,737,056 (Presta), may be performed. Useful effector cells for such assays include PBMC and NK cells. Alternatively, or additionally, ADCC activity of the molecule of interest may be assessed in vivo, e.g., in an animal model such as that disclosed in Clynes et al. PNAS (USA) 95:652-656 (1998).

### Complement Dependent Cytotoxicity

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, e.g., as described in Gazzano-Santoro et al., J. Immunol. Methods 202:163 (1996), may be performed. Polypeptide variants with altered Fc region amino acid sequences (polypeptides with a variant Fc region) and increased or decreased C1q binding capability are described, e.g., in U.S. Pat. No. 6,194,551 B1 and WO 1999/51642. See also, e.g., Idusogie et al. J. Immunol. 164: 4178-4184 (2000).

### Variable Region

The terms “variable region” or “variable domain” as used herein, refer to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6th ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991). As used herein, “heavy chain variable domain (VH)” is used interchangeably with “antibody heavy chain variable domain (VH)”, or “antibody heavy chain variable region (VH)”, or “VH”, or “antibody VH” or “VH domain”, and “light chain variable domain (VL)” is used interchangeably with “antibody light chain variable domain (VH)”, or “antibody light chain variable region (VL)” or “VL”, or “antibody VL” or “VL domain”.

### HVR or CDR

The terms “hypervariable region” or “HVR” as used herein, refer to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra. Framework

The terms “Framework” or “FR” as used herein, refer to variable domain residues other than hypervariable region (HVR) residues. The FR of a variable domain generally consists of four FR domains: FRi, FR2, FR3, and FR4. Accordingly, the HVR and FR sequences generally appear in the following sequence in VH (or VL): FR1-H1(L1)-FR2-H2(L2)-FR3-H3(L3)-FR4.

### Isolated Antibody

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007). Isolated nucleic acid

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-IL-12 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell. The same applies to an anti-IL-22 antibody, etc.

“Isolated nucleic acid encoding a fusion polypeptide that binds IL-12” refers to one or more nucleic acid molecules encoding the polypeptide of formula I (or fragments thereof), including such nucleic acid molecule(s) in a single vector or separate vectors, and such nucleic acid molecule(s) present at one or more locations in a host cell. The same applies to IL-22, etc.

### Vector and Host Cell

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell but may contain mutations. Mutant progeny that have the same function or biological activity as screened or selected for in the originally transformed cell are included herein.

### Individual/Subject

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

### Pharmaceutical Formulation

The term “pharmaceutical formulation” refers to a preparation which is in such form as to permit the biological activity of an active ingredient contained therein to be effective, and which contains no additional components which are unacceptably toxic to a subject to which the formulation would be administered. The “pharmaceutical formulation” may alternatively be called “pharmaceutical composition”.

### Pharmaceutically Acceptable Carrier

A “pharmaceutically acceptable carrier” refers to an ingredient in a pharmaceutical formulation/composition, other than an active ingredient, which is nontoxic to a subject. A pharmaceutically acceptable carrier includes, but is not limited to, a buffer, excipient, stabilizer, or preservative.

### Effective Amount

An “effective amount” of an agent, e.g., a pharmaceutical formulation, refers to an amount effective, at dosages and for periods of time necessary, to achieve the desired therapeutic or prophylactic result.

### Package Insert

The term “package insert” is used to refer to instructions customarily included in commercial packages of therapeutic products, that contain information about the indications, usage, dosage, administration, combination therapy, contraindications and/or warnings concerning the use of such therapeutic products.

### Treatment

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

### Cancer

The terms “cancer” and “cancerous” as used herein, refer to or describe the physiological condition in mammals that is typically characterized by unregulated cell growth/proliferation. Examples of cancer include, but are not limited to, carcinoma, lymphoma (e.g., Hodgkin's and non-Hodgkin's lymphoma), blastoma, sarcoma, and leukaemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, leukaemia and other lymphoproliferative disorders, and various types of head and neck cancer.

### Cell Proliferative Disorder

The terms “cell proliferative disorder” and “proliferative disorder” as used herein, refer to disorders that are associated with some degree of abnormal cell proliferation. In one embodiment, the cell proliferative disorder is cancer.

### B-Cell Neoplasms/Hodgkin's Disease

“B cell neoplasms” include Hodgkin's disease including lymphocyte predominant Hodgkin's disease (LPHD); non-Hodgkin's lymphoma (NHL); follicular centre cell (FCC) lymphomas; acute lymphocytic leukaemia (ALL); chronic lymphocytic leukaemia (CLL); and Hairy cell leukaemia. The non-Hodgkins lymphoma include low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, plasmacytoid lymphocytic lymphoma, mantle cell lymphoma, AIDS-related lymphoma and Waldenstrom's macroglobulinemia. Treatment of relapses of these cancers are also contemplated. LPHD is a type of Hodgkin's disease that tends to relapse frequently despite radiation or chemotherapy treatment. CLL is one of four major types of leukaemia. A cancer of mature B-cells called lymphocytes, CLL is manifested by progressive accumulation of cells in blood, bone marrow and lymphatic tissues. Indolent lymphoma is a slow-growing, incurable disease in which the average patient survives between six and 10 years following numerous periods of remission and relapse.

### Breast Tumour

The term “breast tumour” or “breast cancer” refers to any tumour or cancer of the breast, including, e.g., adenocarcinomas, such as invasive or in situ ductal carcinoma, invasive or in situ lobular carcinoma, medullary carcinoma, colloid carcinoma, and papillary carcinoma; and less prevalent forms, such as cystosarcoma phylloides, sarcomas, squamous cell carcinomas, and carcinosarcomas.

### Colon Tumour

The term “colon tumour” or “colon cancer” refers to any tumour or cancer of the colon (the large intestine from the cecum to the rectum).

### Colorectal Tumour

The term “colorectal tumour” or “colorectal cancer” refers to any tumour or cancer of the large bowel, which includes the colon (the large intestine from the cecum to the rectum) and the rectum, including, e.g., adenocarcinomas and less prevalent forms, such as lymphomas and squamous cell carcinomas.

### Non-Hodgkin's Lymphoma

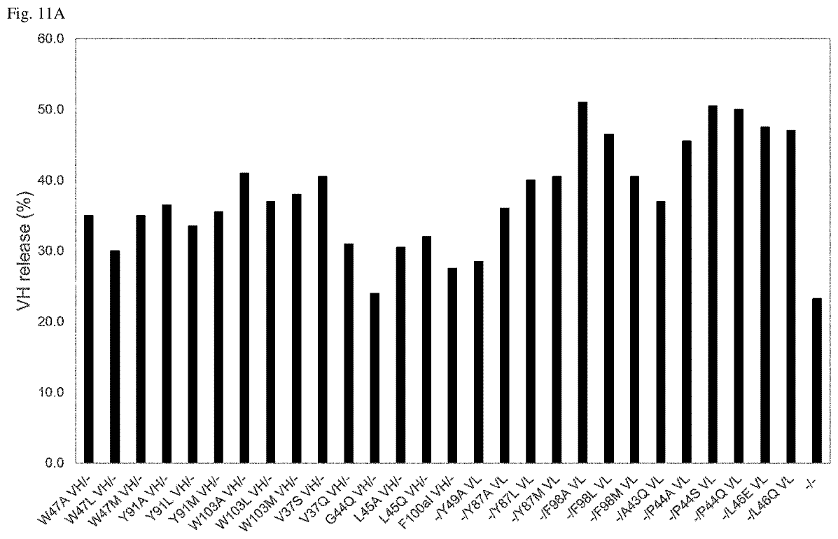

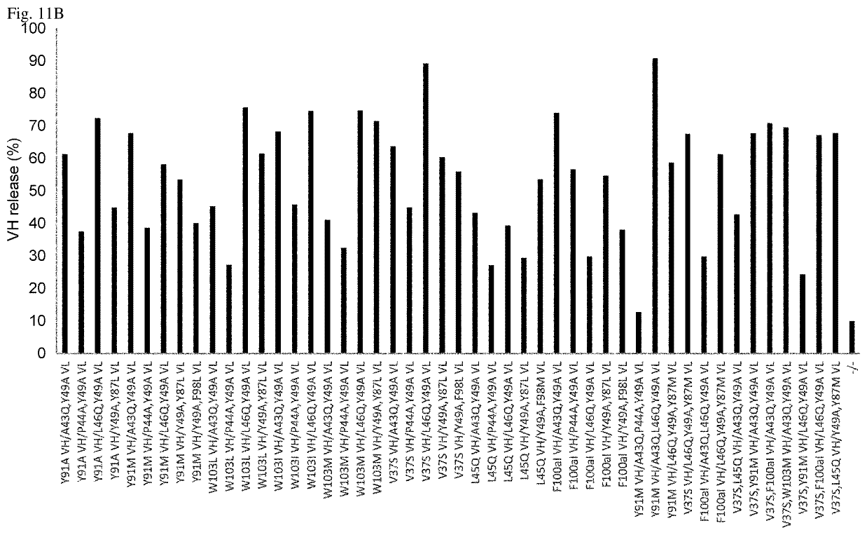

The term “non-Hodgkin's lymphoma” or “NHL”, as used herein, refers to a cancer of the lymphatic system other than Hodgkin's lymphomas. Hodgkin's lymphomas can generally be distinguished from non-Hodgkin's lymphomas by the presence of Reed-Sternberg cells in Hodgkin's lymphomas and the absence of said cells in non-Hodgkin's lymphomas. Examples of non-Hodgkin's lymphomas encompassed by the term as used herein include any that would be identified as such by one skilled in the art (e.g., an oncologist or pathologist) in accordance with classification schemes known in the art, such as the Revised European-American Lymphoma (REAL) scheme as described in Colour Atlas of Clinical Haematology, Third Edition; A. Victor Hoffbrand and John E. Pettit (eds.) (Harcourt Publishers Limited 2000) (see, in particular FIG. 11.57, 11.58 and/or 11.59). More specific examples include, but are not limited to, relapsed or refractory NHL, front line low grade NHL, Stage III/IV NHL, chemotherapy resistant NHL, precursor B lymphoblastic leukaemia and/or lymphoma, small lymphocytic lymphoma, B cell chronic lymphocytic leukaemia and/or prolymphocytic leukaemia and/or small lymphocytic lymphoma, B-cell prolymphocytic lymphoma, immunocytoma and/or lymphoplasmacytic lymphoma, marginal zone B cell lymphoma, splenic marginal zone lymphoma, extranodal marginal zone—MALT lymphoma, nodal marginal zone lymphoma, hairy cell leukaemia, plasmacytoma and/or plasma cell myeloma, low grade/follicular lymphoma, intermediate grade/follicular NHL, mantle cell lymphoma, follicle centre lymphoma (follicular), intermediate grade diffuse NHL, diffuse large B-cell lymphoma, aggressive NHL (including aggressive front-line NHL and aggressive relapsed NHL), NHL relapsing after or refractory to autologous stem cell transplantation, primary mediastinal large B-cell lymphoma, primary effusion lymphoma, high grade immunoblastic NHL, high grade lymphoblastic NHL, high grade small non-cleaved cell NHL, bulky disease NHL, Burkitt's lymphoma, precursor (peripheral) T-cell lymphoblastic leukaemia and/or lymphoma, adult T-cell lymphoma and/or leukaemia, T cell chronic lymphocytic leukaemia and/or prolymphacytic leukaemia, large granular lymphocytic leukaemia, mycosis fungoides and/or Sezary syndrome, extranodal natural killer/T-cell (nasal type) lymphoma, enteropathy type T-cell lymphoma, hepatosplenic T-cell lymphoma, subcutaneous panniculitis like T-cell lymphoma, skin (cutaneous) lymphomas, anaplastic large cell lymphoma, angiocentric lymphoma, intestinal T cell lymphoma, peripheral T-cell (not otherwise specified) lymphoma and angioimmunoblastic T-cell lymphoma.

### Ovarian Cancer

“Ovarian cancer” refers to a heterogeneous group of malignant tumours derived from the ovary. Approximately 90% of malignant ovarian tumours are epithelial in origin; the remainder are germ cell and stromal tumours. Epithelial ovarian tumours are classified into the following histological subtypes: serous adenocarcinomas (constituting about 50% of epithelial ovarian tumours); endometrioid adenocarcinomas (about 20%); mucinous adenocarcinomas (about 10%); clear cell carcinomas (about 5-10%); Brenner (transitional cell) tumours (relatively uncommon). The prognosis for ovarian cancer, which is the sixth most common cancer in women, is usually poor, with five-year survival rates ranging from 5-30%. For reviews of ovarian cancer, see Fox et al. (2002) “Pathology of epithelial ovarian cancer,” in Ovarian Cancer ch. 9 (Jacobs et al., eds., Oxford University Press, New York); Morin et al. (2001) “Ovarian Cancer,” in Encyclopaedic Reference of Cancer, pp. 654-656 (Schwab, ed., Springer-Verlag, New York). The present invention contemplates methods of diagnosing or treating any of the epithelial ovarian tumour subtypes described above, and in particular, the serous adenocarcinoma subtype.

### Relapsed

“Relapsed” refers to the regression of the patient's illness back to its former diseased state, especially the return of symptoms following an apparent recovery or partial recovery. Unless otherwise indicated, relapsed state refers to the process of returning to or the return to illness before the previous treatment including, but not limited to, chemotherapies and stem cell transplantation treatments.

### Refractory

“Refractory” refers to the resistance or non-responsiveness of a disease or condition to a treatment (e.g., the number of neoplastic plasma cells increases even though treatment is given). Unless otherwise indicated, the term “refractory” refers to a resistance or non-responsiveness to any previous treatment including, but not limited to, chemotherapies and stem cell transplantation treatments.

### Stomach Tumour

The term “stomach tumour” or “stomach cancer” as used herein, refers to any tumour or cancer of the stomach, including, e.g., adenocarcinomas (such as diffuse type and intestinal type), and less prevalent forms such as lymphomas, leiomyosarcomas, and squamous cell carcinomas.

### Tumour

The term “tumour” (or “tumor”) as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumour” are not mutually exclusive as referred to herein.

### Inhibiting Cell Growth or Proliferation/Suppressing Cell Growth

“Inhibiting cell growth or proliferation” or “suppressing cell growth” means decreasing a cell's growth or proliferation by at least 10%, 20%, 30%, 40%, 50%, 60%, 70%, 80%, 90%, 95%, or 100%, and includes inducing cell death.

### Chemotherapeutic Agent

A “chemotherapeutic agent” refers to a chemical compound useful in the treatment of cancer. Examples of chemotherapeutic agents include alkylating agents such as thiotepa and cyclophosphamide (CYTOXAN (registered trademark)); alkyl sulfonates such as busulfan, improsulfan and piposulfan; aziridines such as benzodopa, carboquone, meturedopa, and uredopa; ethylenimines and methylol melamines including altretamine, triethylenemelamine, triethylenephosphoramide, triethylenethiophosphoramide and trimethylomelamine; acetogenins (especially bullatacin and bullatacinone); delta-9-tetrahydrocannabinol (dronabinol, MARINOL (registered trademark)); beta-lapachone; lapachol; colchicines; betulinic acid; a camptothecin (including the synthetic analogue topotecan (HYCAMTIN (registered trademark)), CPT-11 (irinotecan, CAMPTOSAR (registered trademark)), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; antibiotics such as the enediyne antibiotics (e. g., calicheamicin, especially calicheamicin gammalI and calicheamicin omegaI1 (see, e.g., Nicolaou et al., Angew. Chem Intl. Ed. Engl., 33: 183-186 (1994)); CDP323, an oral alpha-4 integrin inhibitor; dynemicin, including dynemicin A; an esperamicin; as well as neocarzinostatin chromophore and related chromoprotein enediyne antibiotic chromophores), aclacinomysins, actinomycin, anthramycin, azaserine, bleomycins, cactinomycin, carubicin, carminomycin, carzinophilin, chromomycins, dactinomycin, daunorubicin, detorubicin, 6-diazo-5-oxo-L-norleucine, doxorubicin (including ADRIAMYCIN (registered trademark), morpholino-doxorubicin, cyanomorpholino-doxorubicin, 2-pyrrolino-doxorubicin, doxorubicin HCl liposome injection (DOXIL (registered trademark)), liposomal doxorubicin TLC D-99 (MYOCET (registered trademark)), peglylated liposomal doxorubicin (CAELYX (registered trademark)), and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate, gemcitabine (GEMZAR (registered trademark)), tegafur (UFTORAL (registered trademark)), capecitabine (XELODA (registered trademark)), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatrexate; defofamine; demecolcine; diaziquone; elfornithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitraerine; pentostatin; phenamet; pirarubicin; losoxantrone; 2-ethylhydrazide; procarbazine; PSK (registered trademark) polysaccharide complex (JHS Natural Products, Eugene, OR); razoxane; rhizoxin; sizofiran; spirogermanium; tenuazonic acid; triaziquone; 2,2′,2′-trichlorotriethylamine; trichothecenes (especially T-2 toxin, verracurin A, roridin A and anguidine); urethan; vindesine (ELDISINE (registered trademark), FILDESIN (registered trademark)); dacarbazine; mannomustine; mitobronitol; mitolactol; pipobroman; gacytosine; arabinoside (“Ara-C”); thiotepa; taxoid, e.g., paclitaxel (TAXOL (registered trademark)), albumin-engineered nanoparticle formulation of paclitaxel (ABRAXANE™), and docetaxel (TAXOTERE (registered trademark)); chlorambucil; 6-thioguanine; mercaptopurine; methotrexate; platinum agents such as cisplatin, oxaliplatin (e.g., ELOXATIN (registered trademark)), and carboplatin; vincas, which prevent tubulin polymerization from forming microtubules, including vinblastine (VELBAN (registered trademark)), vincristine (ONCOVIN (registered trademark)), vindesine (ELDISINE (registered trademark), FILDESIN (registered trademark)), and vinorelbine (NAVELBINE (registered trademark)); etoposide (VP-16); ifosfamide; mitoxantrone; leucovorin; novantrone; edatrexate; daunomycin; aminopterin; ibandronate; topoisomerase inhibitor RFS 2000; difluoromethylornithine (DMFO); retinoids such as retinoic acid, including bexarotene (TARGRETIN (registered trademark)); bisphosphonates such as clodronate (for example, BONEFOS (registered trademark) or OSTAC (registered trademark)), etidronate (DIDROCAL (registered trademark)), NE-58095, zoledronic acid/zoledronate (ZOMETA (registered trademark)), alendronate (FOSAMAX (registered trademark)), pamidronate (AREDIA (registered trademark)), tiludronate (SKELID (registered trademark)), or risedronate (ACTONEL (registered trademark)); troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE (registered trademark) vaccine and gene therapy vaccines, for example, ALLOVECTIN (registered trademark) vaccine, LEUVECTIN (registered trademark) vaccine, and VAXID (registered trademark) vaccine; topoisomerase 1 inhibitor (e.g., LURTOTECAN (registered trademark)); rmRH (e.g., ABARELIX (registered trademark)); BAY439006 (sorafenib; Bayer); SU-11248 (sunitinib, SUTENT (registered trademark), Pfizer); perifosine, COX-2 inhibitor (e.g. celecoxib or etoricoxib), proteosome inhibitor (e.g. PS341); bortezomib (VELCADE (registered trademark)); CCI-779; tipifarnib (RI1577); sorafenib, ABT510; Bcl-2 inhibitor such as oblimersen sodium (GENASENSE (registered trademark)); pixantrone; EGFR inhibitors (see definition below); tyrosine kinase inhibitors (see definition below); serine-threonine kinase inhibitors such as rapamycin (sirolimus, RAPAMUNE (registered trademark)); farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents as defined herein include “anti-hormonal agents” or “endocrine therapeutics” which act to regulate, reduce, block, or inhibit the effects of hormones that can promote the growth of cancer. They may be hormones themselves, including, but not limited to: anti-estrogens with mixed agonist/antagonist profile, including, tamoxifen (NOLVADEX (registered trademark)), 4-hydroxytamoxifen, toremifene (FARESTON (registered trademark)), idoxifene, droloxifene, raloxifene (EVISTA (registered trademark)), trioxifene, keoxifene, and selective estrogen receptor modulators (SERMs) such as SERM3; pure anti-estrogens without agonist properties, such as fulvestrant (FASLODEX (registered trademark)), and EM800 (such agents may block estrogen receptor (ER) dimerization, inhibit DNA binding, increase ER turnover, and/or suppress ER levels); aromatase inhibitors, including steroidal aromatase inhibitors such as formestane and exemestane (AROMASIN (registered trademark)), and nonsteroidal aromatase inhibitors such as anastrazole (ARIMIDEX (registered trademark)), letrozole (FEMARA (registered trademark)) and aminoglutethimide, and other aromatase inhibitors include vorozole (RIVISOR (registered trademark)), megestrol acetate (MEGASE (registered trademark)), fadrozole, and 4(5)-imidazoles; lutenizing hormone-releaseing hormone agonists, including leuprolide (LUPRON (registered trademark) and ELIGARD (registered trademark)), goserelin, buserelin, and triptorelin; sex steroids, including progestins such as megestrol acetate and medroxyprogesterone acetate, estrogens such as diethylstilbestrol and premarin, and androgens/retinoids such as fluoxymesterone, all transretinoic acid and fenretinide; onapristone; anti-progesterones; estrogen receptor down-regulators (ERDs); anti-androgens such as flutamide, nilutamide and bicalutamide; and pharmaceutically acceptable salts, acids or derivatives of any of the above; as well as combinations of two or more of the above.

### Cytostatic Agent/Cell-Growth Suppressing Agent

The term “cytostatic agent” or “cell-growth suppressing agent” as used herein, interchangeably, refers to a compound or composition which arrests growth of a cell either in vitro or in vivo. Thus, a cytostatic agent may be one which significantly reduces the percentage of cells in S phase. Further examples of cytostatic agents include agents that block cell cycle progression by inducing G0/G1 arrest or M-phase arrest. The humanized anti-Her2 antibody trastuzumab (HERCEPTIN (registered trademark)) is an example of a cytostatic agent that induces G0/G1 arrest. Classical M-phase blockers include the vincas (vincristine and vinblastine), taxanes, and topoisomerase II inhibitors such as doxorubicin, epirubicin, daunorubicin, etoposide, and bleomycin. Certain agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W.B. Saunders, Philadelphia, 1995), e.g., p. 13. The taxanes (paclitaxel and docetaxel) are anticancer drugs both derived from the yew tree. Docetaxel (TAXOTERE (registered trademark), Rhone-Poulenc Rorer), derived from the European yew, is a semisynthetic analogue of paclitaxel (TAXOL (registered trademark), Bristol-Myers Squibb). Paclitaxel and docetaxel promote the assembly of microtubules from tubulin dimers and stabilize microtubules by preventing depolymerization, which results in the inhibition of mitosis in cells.

### Autoimmune Disease

“Autoimmune disease” refers to a non-malignant disease or disorder arising from and directed against an individual's own tissues. The autoimmune diseases herein specifically exclude malignant or cancerous diseases or conditions, especially excluding B cell lymphoma, acute lymphoblastic leukemia (ALL), chronic lymphocytic leukemia (CLL), Hairy cell leukemia and chronic myeloblastic leukemia. Examples of autoimmune diseases or disorders include, but are not limited to, inflammatory responses such as inflammatory skin diseases including psoriasis and dermatitis (e.g. atopic dermatitis); systemic scleroderma and sclerosis; responses associated with inflammatory bowel disease (such as Crohn's disease and ulcerative colitis); respiratory distress syndrome (including adult respiratory distress syndrome; ARDS); dermatitis; meningitis; encephalitis; uveitis; colitis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE) (including but not limited to lupus nephritis, cutaneous lupus); diabetes mellitus (e.g. Type I diabetes mellitus or insulin dependent diabetes mellitus); multiple sclerosis; Reynaud's syndrome; autoimmune thyroiditis; Hashimoto's thyroiditis; allergic encephalomyelitis; Sjogren's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia (including, but not limited to cryoglobulinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (JTP) or autoimmune thrombocytopenia.

### Immunosuppressive Agents/Anti-Inflammatories

The term “immunosuppressive agent” as used herein for adjunct therapy refers to substances that act to suppress or mask the immune system of the mammal being treated herein. This would include substances that suppress cytokine production, down-regulate or suppress self-antigen expression, or mask the MHC antigens. Examples of such agents include 2-amino-6-aryl-5-substituted pyrimidines (see U.S. Pat. No. 4,665,077); non-steroidal anti-inflammatory drugs (NSAIDs); ganciclovir, tacrolimus, glucocorticoids such as cortisol or aldosterone, anti-inflammatory agents such as a cyclooxygenase inhibitor, a 5-lipoxygenase inhibitor, or a leukotriene receptor antagonist; purine antagonists such as azathioprine or mycophenolate mofetil (MMF); alkylating agents such as cyclophosphamide; bromocryptine; danazol; dapsone; glutaraldehyde (which masks the MHC antigens, as described in U.S. Pat. No. 4,120,649); anti-idiotypic antibodies for MHC antigens and MHC fragments; cyclosporin A; steroids such as corticosteroids or glucocorticosteroids or glucocorticoid analogs, e.g., prednisone, methylprednisolone, including SOLU-MEDROL (registered trademark) methylprednisolone sodium succinate, and dexamethasone; dihydrofolate reductase inhibitors such as methotrexate (oral or subcutaneous); anti-malarial agents such as chloroquine and hydroxychloroquine; sulfasalazine; leflunomide; cytokine or cytokine receptor antibodies including anti-interferon-alpha, -beta, or -gamma antibodies, anti-tumor necrosis factor(TNF)-alpha antibodies (infliximab (REMICADE (registered trademark)) or adalimumab), anti-TNF-alpha immunoadhesin (etanercept), anti-TNF-beta antibodies, anti-interleukin-2 (IL-2) antibodies and anti-IL-2 receptor antibodies, and anti-interleukin-6 (IL-6) receptor antibodies and antagonists (such as ACTEMRA™ (tocilizumab)); anti-LFA-1 antibodies, including anti-CD11a and anti-CD18 antibodies; anti-L3T4 antibodies; heterologous anti-lymphocyte globulin; pan-T antibodies, preferably anti-CD3 or anti-CD4/CD4a antibodies; soluble peptide containing a LFA-3 binding domain (WO 90/08187 published Jul. 26, 1990); streptokinase; transforming growth factor-beta (TGF-beta); streptodornase; RNA or DNA from the host; FK506; RS-61443; chlorambucil; deoxyspergualin; rapamycin; T-cell receptor (Cohen et al., U.S. Pat. No. 5,114,721); T-cell receptor fragments (Offner et al., Science, 251: 430-432 (1991); WO 90/11294; Ianeway, Nature, 341: 482 (1989); and WO 91/01133); BAFF antagonists such as BAFF antibodies and BR3 antibodies and zTNF4 antagonists (for review, see Mackay and Mackay, Trends Immunol., 23:113-5 (2002) and see also definition below); biologic agents that interfere with T cell helper signals, such as anti-CD40 receptor or anti-CD40 ligand (CD154), including blocking antibodies to CD40-CD40 ligand (e.g., Durie et al., Science, 261: 1328-30 (1993); Mohan et al., J. Immunol., 154: 1470-80 (1995)) and CTLA4-Ig (Finck et al., Science, 265: 1225-7 (1994)); and T-cell receptor antibodies (EP 340,109) such as T10B9. Some preferred immunosuppressive agents herein include cyclophosphamide, chlorambucil, azathioprine, leflunomide, MMF, or methotrexate.

### II. Exemplary Embodiments of Present Invention

In one embodiment, the present invention relates to a fusion protein, comprising a polypeptide comprising at least one ligand-binding moiety comprising a ligand-binding domain comprising an antibody variable region, a protease cleavage site and at least one ligand that is connected to a C-terminal region of the ligand binding moiety by a non-cleavable peptide linker and wherein (a) in a first state, the ligand is bound by the ligand binding domain and the biological activity of the ligand is attenuated, and in a second state, the biological activity of the ligand is restored, and (b) the fusion protein in the first state has a longer half-life in blood than in the second state, and (c) switching from the first state to the second state is mediated by the presence of a protease.

The difference between the “first state” and “second state” may be the absence/presence of protease cleavage. The phrase “in a first state” may be rephrased as “before the protease cleavage site is cleaved by the protease” or “when the protease cleavage site is uncleaved by the protease”, or “uncleaved state”. The phrase “in a second state” may be rephrased as “after the protease cleavage site is cleaved by the protease” or “when the protease cleavage site is cleaved by the protease”, or “cleaved state”. The same applies to other embodiments described herein.

In one embodiment, the present invention relates to a bivalent homodimer fusion protein comprising two polypeptides, each comprising:
(i) a ligand-binding moiety comprising a ligand-binding domain and a constant region
(ii) a first peptide linker comprising a protease cleavage site and connects the ligand-binding domain to the constant region
(iii) said constant region comprising a second peptide linker and optionally one or more amino acid residues which are modified from or to cysteine
(iv) a ligand moiety connected to a C-terminal region of the constant region by a third peptide linker, and;
wherein (a) in a first state, the ligand moiety is bound by the ligand-binding domain and the biological activity of the ligand is attenuated, and in a second state, the biological activity of the ligand is restored, and (b) the fusion protein in the first state has a longer half-life in blood than in the second state, and (c) switching from the first state to the second state is mediated by the presence of a protease.

In one embodiment, the present invention relates to a bivalent homodimer fusion protein comprising an IgG antibody-like polypeptide fused to a ligand moiety, comprising
(i) a first peptide linker comprising a protease cleavage site between the boundary of (ia) VH and CH1 or (ib) VL and CL,
(ii) a second peptide linker introduced in the hinge region connecting the CH1 to Fc region of the antibody and optionally comprising one or more amino acid residues which are modified from or to cysteine and;
(iii) a third peptide linker connecting the ligand moiety to C-terminus of the Fc region of the antibody
   wherein (a) in a first state, the ligand moiety is bound by the antibody variable region and the biological activity of the ligand is attenuated, and in a second state, the biological activity of the ligand is restored, and (b) the fusion protein in the first state has a longer half-life in blood than in the second state, and (c) switching from the first state to the second state is mediated by the presence of a protease.

In one embodiment, the present invention relates to a bivalent homodimer fusion protein comprising two polypeptides, each represented by the general formula (I), from the N- to the C-terminus:

[Ligand-binding domain]-[Lx]-[Cx]-[Ly]-[Ligand moiety] (I)
wherein:
Lx represents a peptide linker comprising a protease cleavage site,
Cx represents a constant region comprising a second peptide linker and optionally one or more amino acid residues which are modified from or to cysteine;
Ly represents a third peptide linker,
and wherein (a) in a first state, the ligand moiety is bound by the ligand-binding domain and the biological activity of the ligand moiety is attenuated, and in a second state, the biological activity of the ligand is restored, and (b) the fusion protein in the first state has a longer half-life in blood than in the second state, and (c) switching from the first state to the second state is mediated by the presence of a protease.

In one embodiment, the present invention relates to a bivalent homodimer fusion protein comprising a full-length IgG antibody comprising an antigen-binding domain, wherein the antigen-binding domain comprises a variable region, wherein the variable region comprises a heavy chain variable domain (VH) and a light chain variable domain (VL) that associates with each other, and comprises (a) a protease cleavage site at the boundary between VH and CH1 or VL and CL of the variable region, and (b) a ligand moiety binding to the variable region, and wherein (a) in a first state, the ligand moiety is bound by the variable region and the biological activity of the ligand moiety is attenuated, and in a second state, the biological activity of the ligand moiety is restored, and (b) the fusion protein in the first state has a longer half-life in blood than in the second state, and (c) switching from the first state to the second state is mediated by the presence of a protease. As used herein, the full-length IgG antibody includes an IgG antibody-like polypeptide as described herein.

In one embodiment, the present invention relates to a bivalent homodimer fusion protein comprising a IgG antibody-like polypeptide comprising an antigen-binding domain, wherein the antigen-binding domain comprises a variable region, wherein the variable region comprises a heavy chain variable domain (VH) and a light chain variable domain (VL) that associates with each other, and comprises (a) a protease cleavage site at the boundary between VH and CH1, or VL and CL, of the variable region, and (b) a ligand binding to said variable region, and wherein upon protease cleavage, (i) either the VH or the VL dissociates from the fusion protein, and (ii) the ligand dissociates from the variable region, and wherein the dissociation described in (i) is promoted by at least one amino acid modification performed at the interface between VH and VL that reduces association between VH and VL in the cleaved state compared to the uncleaved state, and wherein said amino acid residue(s) for modification resides in the Framework region (FR).

In one embodiment, the present invention relates to a bivalent homodimer fusion protein comprising two polypeptides, each represented by the general formula (I), from the N- to the C-terminus:

[ligand-binding domain]-[Lx]-[Cx]-[Ly]-[ligand moiety] (I)
wherein:
Lx represents a peptide linker comprising a protease cleavage site,
Cx represents a constant region comprising a second peptide linker and optionally one or more amino acid residues which are modified from or to cysteine;
Ly represents a third peptide linker,
and wherein the ligand-binding domain comprises a heavy chain variable domain (VH) and a light chain variable domain (VL), and wherein the ligand-binding domain comprises at least one amino acid modification that reduces association between VH and VL in the presence of a protease that catalyses the cleavage of said protease cleavage site (“cleaved state”) compared to in the absence of said protease (“uncleaved state”), and wherein said amino acid residue(s) for modification resides in the Framework region (FR).

In one embodiment, the present invention relates to a polypeptide or antibody comprising at least one antigen-binding domain comprising a protease cleavage site, whereupon cleavage at the protease cleavage site, an antibody domain adjacent to the protease cleavage site dissociates. As used herein, the term “antibody domain” refers to a molecule other than an intact antibody, i.e. a portion of an antibody, including but not limited to antibody fragments, such as VH, VL, VHH, CH1, CH2, CH3, CL, Fv, Fab, Fab′, Fab′-SH, F(ab′)2, scFv etc.

In one embodiment, upon protease cleavage at the protease cleavage site(s), a portion of the polypeptide or antibody or antibody domain thereof dissociates from the rest of the polypeptide or antibody. The dissociation is promoted by at least one amino acid modification performed at the interface between said portion or domain and a corresponding interacting portion or domain. For example, where the antibody domain is VH, a corresponding interacting domain thereof is VL, and where the antibody domain is VL, a corresponding interacting domain thereof is VH.

In the present specification, several molecular formats are included. As used herein, the terms “ligand” and “antigen” are used interchangeably and refer broadly to all molecules that can be specifically bound by a ligand-binding domain or antigen-binding domain. The terms “Ligand-binding domains” and “antigen-binding domains” refer to molecules capable of binding to a ligand or an antigen respectively. In the case where the ligand-binding domain comprises an antibody fragment thereof capable of binding to a ligand and neutralising the biological activity of the ligand, the ligand-binding domain may be used interchangeably with an antigen-binding domain.

### Protease Cleavage Site

In the present invention, the ligand-binding domain/moiety/molecule comprises at least one protease cleavage site. The protease cleavage site may be placed anywhere within the ligand-binding domain/moiety/molecule as long as upon protease cleavage, the ligand becomes released or unbound from the ligand-binding domain and the biological activity of the ligand to bind its binding partner is restored. As used herein, the phrase “release/releasing the ligand moiety/molecule” or “the ligand moiety/molecule is released” means that the ligand moiety/molecule becomes able to exert and/or increase its biological activity through interacting with a binding partner thereof compared with the biological activity of the ligand moiety/molecule bound with the uncleaved ligand-binding domain/moiety/molecule, but does not refer to or pose limitations on any particular level of release or any particular mode of action by which the ligand moiety/molecule is released.

For example, a protease cleavage site may be placed near or even within the ligand-binding domain in the ligand-binding moiety/molecule. Protease cleavage at the protease cleavage site can affect, e.g., restore, the biological activity of a ligand which can be bound by the moiety/molecule. As used herein, for example, the phrase “biological activity is restored” refers to the state when the ligand transits from a (first) state when it is bound to the ligand-binding moiety/molecule in the uncleaved state and unable to interact with a binding partner (i.e., the biological activity is attenuated due to the absence of the interaction) to a (second) state when it is not bound to the ligand-binding domain in the cleaved state and able to interact with a binding partner and exert its biological activity thereof. The physiological activity of the ligand to bind its binding partner is attenuated in the first state when it is bound by the ligand-binding domain and is restored in the second state when it is unbound from the ligand-binding domain in the presence of protease.

In some embodiments, in the presence of a protease, a ligand moiety/molecule linked to or bound by a ligand-binding moiety/molecule may be released from the ligand-binding domain in the ligand-binding moiety/molecule, due to the cleavage at a protease cleavage site placed within or near the ligand-binding domain in the ligand-binding moiety/molecule. In some embodiments, even after cleavage, the ligand moiety/molecule may still be linked to the C-terminal region (e.g., Fc region/domain) of the ligand-binding moiety/molecule. In some embodiments, in the presence of a protease, a ligand moiety/molecule may be released entirely from the ligand-binding moiety/molecule, due to the cleavage at a protease cleavage site placed between the ligand moiety/molecule and the C-terminal region (e.g., Fc region/domain) of the ligand-binding moiety/molecule. In some embodiments, after cleavage, the ligand moiety/molecule is no longer linked to the C-terminal region (e.g., Fc region/domain) of the ligand-binding moiety/molecule.

In one embodiment, the ligand-binding moiety/molecule binds to the ligand moiety/molecule more weakly (i.e. ligand binding is attenuated) in a cleaved state compared with an uncleaved state. In another embodiment, the ligand-binding moiety/molecule does not bind to the ligand or ligand moiety (i.e., ligand binding is abolished) in a cleaved state compared with an uncleaved state. In an embodiment in which the ligand-binding moiety/molecule binds to the ligand moiety/molecule by antigen-antibody reaction, the attenuation of the ligand binding or lack thereof, can be evaluated on the basis of the biological activity of the ligand-binding moiety/molecule.

In one embodiment, the antigen-binding domain binds to the ligand moiety/molecule more weakly (i.e. ligand binding is attenuated) in a cleaved state compared with an uncleaved state. In another embodiment, the antigen-binding domain does not bind to the ligand or ligand moiety (i.e., ligand binding is abolished) in a cleaved state compared with an uncleaved state. In this case, the antigen-binding domain binds the ligand moiety/molecule by antigen-antibody reaction, the attenuation of the ligand binding, or lack thereof, can be evaluated on the basis of the biological activity of the ligand-binding moiety/molecule.

The phrase “ligand binding is attenuated” means that the amount of a test ligand binding molecule bound with the ligand is, for example, 90% or less, 80% or less, 70% or less, 60% or less, 50% or less, preferably 45% or less, 40% or less, 35% or less, 30% or less, 20% or less, or 15% or less, particularly preferably 10% or less, 9% or less, 8% or less, 7% or less, 6% or less, 5% or less, 4% or less, 3% or less, 2% or less, or 1% or less, of the amount of a control ligand binding molecule bound with the ligand on the basis of the measurement method described above. The desired index may be appropriately used as an index for binding activity. For example, a dissociation constant (KD) may be used. In the case of using a dissociation constant (KD) as an index for evaluating binding activity, a larger dissociation constant (KD) of the test ligand binding molecule for the ligand than that of a control ligand binding molecule for the ligand means that the test ligand binding molecule has weaker binding activity against the ligand than that of the control ligand binding molecule. The phrase “ligand binding function is attenuated” means that the dissociation constant (KD) of the test ligand binding molecule for the ligand is, for example, at least 2 times, preferably at least 5 times or at least 10 times, particularly preferably at least 100 times the dissociation constant (KD) of the control ligand binding molecule for the ligand.

Examples of the control ligand binding molecule include an uncleaved form of the ligand-binding moiety/molecule or an uncleaved form of the antibody or antibody fragment.

In some embodiments of the present invention, the biological activity of the ligand moiety/molecule is attenuated by binding to the ligand-binding domain of the uncleaved ligand-binding moiety/molecule. Examples of the embodiments in which the biological activity of the ligand is attenuated include, but are not limited to, embodiments in which the binding of the ligand moiety/molecule to the ligand-binding domain of the uncleaved ligand binding moiety/molecule substantially or significantly interferes or competes with the binding of the ligand to its binding partner. In the case of using an antibody or a fragment thereof having ligand neutralizing activity as the ligand-binding moiety/molecule, the ligand-binding moiety/molecule bound with the ligand is capable of attenuating, and to the larger extent of attenuating, i.e. inhibiting the biological activity of the ligand by exerting its neutralising activity.

In some embodiments of the present invention, the biological activity of the ligand moiety/molecule is attenuated by binding to the antigen-binding domain of the antibody. Examples of the embodiments in which the biological activity of the ligand is attenuated include, but are not limited to, embodiments in which the binding of ligand to the antigen-binding domain of the uncleaved antibody substantially or significantly interferes or competes with the binding of the ligand to its binding partner. Binding of ligand to the antigen-binding domain attenuates or inhibits the biological activity of the ligand by exerting the neutralising activity via antigen-antibody binding interaction.

In one embodiment of the present invention, preferably, the uncleaved ligand-binding moiety/molecule can sufficiently neutralize the biological activity of the ligand moiety by binding to the ligand moiety. Specifically, the biological activity of the ligand moiety/molecule when bound with the uncleaved ligand-binding moiety/molecule is preferably lower than that of the ligand moiety/molecule when unbound from the uncleaved ligand-binding moiety/molecule. The biological activity of the ligand when bound with the uncleaved ligand binding molecule can be, for example, 90% or less, preferably 80% or less, 70% or less, 60% or less, 50% or less, 40% or less, or 30% or less, particularly preferably 20% or less, 10% or less, 9% or less, 8% or less, 7% or less, 6% or less, 5% or less, 4% or less, 3% or less, 2% or less, or 1% or less, of the biological activity of the ligand when unbound from the uncleaved ligand binding molecule, though not limited thereto. The administration of the ligand-binding moiety/molecule, which sufficiently neutralizes the biological activity of the ligand, can be expected to prevent the ligand from exerting its biological activity before arriving at a target tissue.

In another embodiment of the present invention, preferably, the uncleaved antigen-binding domain can sufficiently neutralize the biological activity of the ligand moiety by binding to the ligand moiety. Specifically, the biological activity of the ligand moiety/molecule when bound with the uncleaved antigen-binding domain is preferably lower than that of the ligand moiety/molecule when unbound from the uncleaved antigen-binding domain. The biological activity of the ligand when bound with the uncleaved antigen-binding domain can be, for example, 90% or less, preferably 80% or less, 70% or less, 60% or less, 50% or less, 40% or less, or 30% or less, particularly preferably 20% or less, 10% or less, 9% or less, 8% or less, 7% or less, 6% or less, 5% or less, 4% or less, 3% or less, 2% or less, or 1% or less, of the biological activity of the ligand when unbound from the uncleaved antigen-binding domain, though not limited thereto. The administration of the antigen-binding domain, which sufficiently neutralizes the biological activity of the ligand, can be expected to prevent the ligand from exerting its biological activity before arriving at a target tissue.

Alternatively, the present invention provides methods for neutralizing the biological activity of a ligand. The methods of the present invention comprise the steps of contacting a ligand-binding molecule of the present invention with a ligand whose biological activity should be neutralized and collecting the product of binding of the two molecules. Cleavage of the ligand-binding molecule in the collected binding product can restore the neutralized biological activity of the ligand. Thus, the methods for neutralizing the biological activity of a ligand according to the present invention may further comprise the step of restoring the biological activity of the ligand by cleaving the ligand-binding molecule in the binding product which consists of the ligand and the ligand-binding molecule (in other words, cancelling the neutralizing activity of the ligand-binding molecule).

In one embodiment of the present invention, the binding activity of the cleaved ligand binding moiety or molecule against the ligand moiety or molecule is preferably lower than that of an in vivo natural ligand binding partner (e.g., natural receptor for the ligand) against the ligand. The binding activity of the cleaved ligand binding moiety/molecule against the ligand moiety/molecule exhibits, for example, 90% or less, preferably 80% or less, 70% or less, 60% or less, 50% or less, 40% or less, or 30% or less, particularly preferably 20% or less, 10% or less, 9% or less, 8% or less, 7% or less, 6% or less, 5% or less, 4% or less, 3% or less, 2% or less, or 1% or less, of the amount of the ligand bound with the in vivo natural binding partner (per unit binding partner), though not limited thereto. The desired index may be appropriately used as an index for binding activity. For example, a dissociation constant (KD) may be used. In the case of using a dissociation constant (KD) as an index for evaluating binding activity, a larger dissociation constant (KD) of the cleaved ligand binding moiety/molecule for the ligand than that of the in vivo natural binding partner for the ligand means that the cleaved ligand binding molecule has weaker binding activity against the ligand than that of the in vivo natural binding partner. The dissociation constant (KD) of the cleaved ligand binding molecule for the ligand is, for example, at least 1.1 times, preferably at least 1.5 times, at least 2 times, at least 5 times, or at least 10 times, particularly preferably at least 100 times the dissociation constant (KD) of the in vivo natural binding partner for the ligand. The ligand binding molecule having only low binding activity against the ligand or hardly having binding activity against the ligand after cleavage guarantees that the ligand is released by the cleavage of the ligand binding molecule and can be expected to be prevented from binding to another ligand molecule again.

The ligand desirably restores the suppressed biological activity after cleavage of the ligand binding molecule. Desirably, the ligand binding of the cleaved ligand binding molecule is attenuated so that the ligand biological activity-inhibiting function of the ligand binding molecule is also attenuated. Those skilled in the art can confirm the biological activity of the ligand by a known method, for example, a method of detecting the binding of the ligand to its binding partner as disclosed herein.

In the present specification, the phrase “attenuated binding activity” as used herein, when referring to the binding activity of the ligand for its binding partner, refers to reduced or decreased binding activity when compared with the binding activity of the ligand in the uncleaved state of the fusion polypeptide, and the degree of reduction or decrease is not limited, and includes complete abolishment of activity. Similarly, the phrase “suppressed biological activity” and “neutralising the biological activity of the ligand” may be used herein interchangeably to demonstrate a reduction, including and not limited to complete elimination, of the binding activity of the ligand for its binding partner when the ligand is bound to the ligand-binding domain of the fusion polypeptide before protease cleavage.

In the present specification, the phrase “biological activity is restored” refers to the state when the ligand transits from a (first) state when it is bound to the ligand-binding moiety in the uncleaved state and unable to interact with a binding partner to a (second) state when it is not bound to the ligand binding moiety in the cleaved state and able to interact with a binding partner and exert its biological activity thereof. The term “restored” refers to the return of the ability of the ligand to interact with a binding partner and exert its biological activity thereof, where this ability had been inhibited when the ligand was bound to the ligand binding domain in the uncleaved state. It includes any degree of interaction or increased interaction with a binding partner sufficient to exert its biological activity thereof upon binding. In some embodiments, the ligand-binding moiety/molecule comprises a protease cleavage site placed within or near the ligand-binding domain in the ligand-binding moiety. In the presence of protease, the ligand becomes unbound to the ligand-binding moiety and free to interact with a binding partner and exert its biological activity. In some embodiments, the interaction of ligand to a binding partner to exert its biological activity occurs while the ligand remains bound by a non-cleavable peptide linker to the C-terminal end of the Fc region of the ligand-binding moiety. The term “biological activity” as used herein, includes but is not limited to, the physiological activity of the ligand (e.g. ligand interaction with its natural binding partner such as a ligand receptor).

The biological activity of the ligand to bind its ligand binding partner can be confirmed by well-known methods such as FACS, ELISA, BIACORE using ALPHA (ALPHA (amplified luminescent proximity homogeneous assay) screening or surface plasmon resonance (SPR) phenomena, or BLI (bio-layer interferometry) (Octet) (Proc. Natl. Acad. Sci. USA (2006) 103 (11), 4005-4010). The ALPHA screening is carried out on the basis of the following principle according to ALPHA technology using two beads, a donor and an acceptor. Luminescence signals are detected only when these two beads are located in proximity through the interaction between a molecule bound with the donor bead and a molecule bound with the acceptor bead. A laser-excited photosensitizer in the donor bead converts ambient oxygen to singlet oxygen in an excited state. The singlet oxygen diffuses around the donor bead and reaches the acceptor bead located in proximity thereto to thereby cause chemiluminescent reaction in the bead, which finally emits light. In the absence of the interaction between the molecule bound with the donor bead and the molecule bound with the acceptor bead, no chemiluminescent reaction occurs because singlet oxygen produced by the donor bead does not reach the acceptor bead.

For example, a biotin-labeled ligand binding partner is bound to the donor bead, while a glutathione S transferase (GST)-tagged ligand is bound to the acceptor bead. In the absence of an untagged competitor ligand binding partner, the ligand binding partner interacts with the ligand to generate signals of 520 to 620 nm. The untagged ligand binding partner competes with the tagged ligand binding partner for the interaction with the ligand. Decrease in fluorescence resulting from the competition can be quantified to determine relative binding affinity. The biotinylation of the ligand binding partner such as an antibody using sulfo-NHS-biotin or the like is known in the art. A method which involves, for example: fusing a polynucleotide encoding the ligand in flame with a polynucleotide encoding GST; expressing a GST-fused ligand from cells or the like carrying a vector that permits expression of the resulting fusion gene; and purifying the GST-fused ligand using a glutathione column can be appropriately adopted as a method for tagging the ligand with GST. The obtained signals are preferably analyzed using, for example, software GRAPHPAD PRISM (GraphPad Software, Inc., San Diego) adapted to a one-site competition model based on nonlinear regression analysis.

One (ligand) of the substances between which the interaction is to be observed is immobilized onto a thin gold film of a sensor chip. The sensor chip is irradiated with light from the back such that total reflection occurs at the interface between the thin gold film and glass. As a result, a site having a drop in reflection intensity (SPR signal) is formed in a portion of reflected light. The other (analyte) of the substances between which the interaction is to be observed is flowed on the surface of the sensor chip and bound to the ligand so that the mass of the immobilized ligand molecule is increased to change the refractive index of the solvent on the sensor chip surface. This change in the refractive index shifts the position of the SPR signal (on the contrary, the dissociation of the bound molecules gets the signal back to the original position). The Biacore system plots on the ordinate the amount of the shift, i.e., change in mass on the sensor chip surface, and displays time-dependent change in mass as assay data (sensorgram). Kinetics: an association rate constant (ka) and a dissociation rate constant (kd) are determined from the curve of the sensorgram, and a dissociation constant (KD) is determined from the ratio between these constants. Inhibition assay or equilibrium analysis is also preferably used in the BIACORE method. Examples of the inhibition assay are described in Proc. Natl. Acad. Sci. USA (2006) 103 (11), 4005-4010, and examples of the equilibrium analysis are described in Method Enzymol. 2000; 323: 325-40.

The phrase “biological activity is restored” as used herein, does not place a limitation on the degree of binding of the ligand to its binding partner as long as biological activity resulting from the binding is observed in any measurement methods, such as those described above. It can include 1% or more, 2% or more, 3% or more, 4% or more, 5% or more, 6% or more, 7%, or more 8% or more, 9% or more, 10% or more, 20% or more, 30% or more, 40% or more, 50% or more, 60% or more, 70% or more, 80% or more, or 90% or more increase in interaction between the ligand and its binding partner when comparing the ligand in a cleaved state and uncleaved state. The desired index may be appropriately used as an index for binding activity. For example, an association rate constant (Kon) may be used. In the case of using an association constant (Kon), a larger association constant of the ligand binding partner for a test ligand (i.e. restored ligand in a cleaved state) than that of a control ligand (i.e. ligand in an uncleaved state) means stronger ligand binding activity of the test ligand for the ligand binding partner than that of the control ligand. In some embodiments, the association constant is at least 2 times, preferably at least 5 times, or at least 10 times, particularly preferably at least 100 times that of the control ligand for the ligand binding partner.

In the case of detecting the biological activity of the ligand using, for example, Octet, the antibody for ligand detection that recognizes the ligand is biotinylated and contacted with a biosensor. Then, binding to the ligand in a sample can be measured to detect the restoration of the ligand binding activity. Specifically, the amount of the ligand is measured in a sample containing the ligand binding molecule before protease treatment or after protease treatment and the ligand, using the antibody for ligand detection. The amount of the ligand detected in the sample can be compared between before and after protease treatment to detect the release of the ligand. Alternatively, the amount of the ligand is measured in a sample containing protease, the ligand binding molecule, and the ligand and a sample containing the ligand binding molecule and the ligand without containing protease, using the antibody for ligand detection. The amount of the ligand detected in the sample can be compared between the presence and absence of protease to determine the restoration of ligand binding ability of the ligand moiety. When the ligand binding molecule is fused with the ligand to form a fusion protein, the amount of the ligand is measured in a sample containing the fusion protein before protease treatment or after protease treatment, using the antibody for ligand detection. The amount of the ligand detected in the sample can be compared between before and after protease treatment to determine the restoration of ligand binding ability of the ligand. Alternatively, the amount of the ligand is measured in a sample containing protease and the fusion protein and a sample containing the fusion protein without containing protease, using the antibody for ligand detection. The amount of the ligand detected in the sample can be compared between the presence and absence of protease to determine restoration of the ligand binding ability of the ligand. More specifically, the restoration of ligand binding activity of the ligand can be detected by a method described in Examples of the present application.

In some embodiments, the physiological activity of the ligand (i.e. ligand interaction with its natural binding partner such as the ligand receptor) is attenuated upon binding to the ligand-binding domain, the restoration of this physiological activity of the ligand can be detected by a method of measuring the physiological activity of the ligand in a sample. Specifically, the physiological activity of the ligand can be measured in a sample containing the ligand binding molecule before protease treatment or after protease treatment and the ligand and compared between before and after protease treatment to detect the restoration of its binding ability. Alternatively, the physiological activity of the ligand can be measured in a sample containing protease, the ligand binding molecule, and the ligand and a sample containing the ligand binding molecule and the ligand without containing protease and compared between these samples to detect the restoration of the binding ability of the ligand. When the ligand binding molecule is fused with the ligand to form a fusion protein, the physiological activity of the ligand can be measured in a sample containing the fusion protein before protease treatment or after protease treatment and compared between before and after protease treatment to detect the restoration of its binding ability. Alternatively, the physiological activity of the ligand can be measured in a sample containing protease and the fusion protein and a sample containing the fusion protein without containing protease and compared between these samples to detect the restoration of its binding ability.

In some embodiments of the present invention, the uncleaved ligand binding molecule forms a complex with the ligand through antigen-antibody binding. In a more specific embodiment, the complex of the ligand binding molecule and the ligand is formed through a noncovalent bond, for example, antigen-antibody binding, between the ligand binding molecule and the ligand.

In some embodiments, an uncleaved ligand binding molecule is fused with a ligand molecule to form a fusion protein. The ligand binding domain of the ligand binding moiety and the ligand moiety in the fusion protein further interact with each other through antigen-antibody binding. The ligand binding molecule and the ligand can be fused via a peptide linker. Even when the ligand binding molecule and the ligand in the fusion protein are fused via a peptide linker, the noncovalent bond still exists between the ligand binding domain of the ligand binding moiety and the ligand moiety. In other words, even in the embodiments in which the ligand binding molecule is fused with the ligand, the noncovalent bond between the ligand binding domain of the ligand binding moiety and the ligand moiety is similar to that in the case where the ligand binding molecule is not fused with the ligand. The noncovalent bond is attenuated by the cleavage of the ligand binding moiety/molecule. In short, the ligand binding of the ligand binding moiety/molecule is attenuated.

In some embodiments, in the fusion protein of the present invention, the ligand moiety or molecule is connected to a C-terminal region of the ligand-binding moiety or molecule via a peptide linker. As used herein, the term “C-terminal region” refers to a region of a polypeptide that extends from an internal amino acid residue in the polypeptide to the C-terminal amino acid residue of the polypeptide. In certain embodiments where the ligand-binding moiety/molecule is, for example, in the form of an antibody or in the form of an antibody fragment that contains an Fc region, the C-terminal region of the ligand-binding moiety/molecule typically refers to a region of the 1st to 250th amino acid residues from the C-terminus of the ligand-binding moiety/molecule. In a preferred embodiment, the ligand moiety/molecule is connected to the C-terminal amino acid residue of the ligand-binding moiety/molecule via a peptide linker. The peptide linker may be attached to the ligand moiety/molecule and to the C-terminal region of the ligand-binding moiety/molecule by any covalent bonds such as peptide bonds. The length of the peptide linker is not particularly limited as long as it allows the ligand moiety/molecule to bind to the ligand-binding domain in the ligand-binding moiety/molecule. The above-mentioned peptide linker may or may not contain a protease cleavage site. In a preferred embodiment, the above-mentioned peptide linker does not contain a protease cleavage site.

In some aspects, the ligand moiety/molecule of the present invention is IL-12. In one embodiment, the ligand moiety/molecule of the present invention is IL-12 and the IL-12 is connected with C-terminal amino acid residue of the ligand-binding moiety/molecule via a peptide linker attached to p35 subunit of IL-12 or p40 subunit of IL-12.

In one embodiment, the ligand moiety/molecule of the present invention is IL-12 and the IL-12 is connected with C-terminal amino acid residue of the ligand-binding moiety/molecule via a peptide linker attached to the N-terminus of p35 subunit of IL-12 or p40 subunit of IL-12. In some aspects, the ligand moiety/molecule of the present invention is IL-22. In one embodiment, the ligand moiety/molecule of the present invention is IL-22 and the IL-22 is connected with C-terminal amino acid residue of the ligand-binding moiety/molecule via a peptide linker attached to IL-22. In one embodiment, the ligand moiety/molecule of the present invention is IL-22 and the IL-22 is connected with C-terminal amino acid residue of the ligand-binding moiety/molecule via a peptide linker attached to the N-terminus of IL-22. In one embodiment, the ligand-binding domain of the present invention is connected to a hinge region comprised in the ligand-binding moiety via a peptide linker. In a preferred embodiment, the ligand-binding moiety of the present invention may further comprise a CH1 region which is connected to a hinge region via a peptide linker. The peptide linker can be inserted between CH1 and hinge on either side of the linker. In some embodiments, the fusion protein (or ligand-binding moiety) of the invention comprises a constant region comprising a peptide linker. In some embodiments, the constant region comprises a hinge region comprising a peptide linker. The peptide linker may be comprised at any position before/within the hinge region. The peptide linker may be comprised between CH1 and the hinge region, i.e., before the amino acid sequence EPKSC (SEQ ID NO: 936) in the hinge region (note: the initial residue (E) is at position 216 (EU numbering)). The peptide linker may be comprised after the amino acid sequence EPKSC (SEQ ID NO: 936) in the hinge region. Examples of the position of the peptide linker include, but are not limited to, the following:

[Peptide linker]EPKSCDKTHTCPPCP (see SEQ ID NO:
901; examples include “C1” type);
EPKSC[Peptide linker]DKTHTCPPCP (see SEQ ID NO:
905; examples include “C2” type);
and
[Peptide linker]EPKSSDKTHTCPPCP (see SEQ ID NOs:
908 and 910; examples include “C3” and “C4”
types);
and
EPKSCDKTHT[Peptide linker]CPPCP (see SEQ ID NO:
932; examples include “C5” type).

In some embodiments, the peptide linker ([Peptide linker] indicated above) is a GS linker mentioned herein, such as (GS)₂, (GGGGS: SEQ ID NO: 141)₂.

The suitable peptide linker above may be readily selected and can be preferably selected from among different lengths such as 1 amino acid (Gly, etc.) to 300 amino acids, 2 amino acids to 200 amino acids, or 3 amino acids to 100 amino acids including 4 amino acids to 100 amino acids, 5 amino acids to 100 amino acids, 5 amino acids to 50 amino acids, 5 amino acids to 30 amino acids, 5 amino acids to 25 amino acids, or 5 amino acids to 20 amino acids.

Examples of the peptide linker include, but are not limited to, glycine polymers (G)n, glycine-serine polymers (including e.g., (GS)n, (GGGGS: SEQ ID NO: 141)n and (GGGS: SEQ ID NO: 136)n, wherein n is an integer of at least 1), glycine-alanine polymers, alanine-serine polymers, and other flexible linkers well known in conventional techniques.

Examples of the peptide linker can include, but are not limited to,

Gly Gly Gly Gly Ser (GGGGS, SEQ ID NO: 141)
(Gly Gly Gly Gly Ser (GGGGS, SEQ ID NO: 141))n
Gly Gly Gly Gly Ala (GGGGA, SEQ ID NO: 893)
Gly Gly Gly Gly Glu (GGGGE, SEQ ID NO: 894)
Gly Gly Gly Ser (GGGS, SEQ ID NO: 136)
(Gly Gly Gly Ser (GGGS, SEQ ID NO: 136))n
Gly Gly Gly Ala (GGGA, SEQ ID NO: 895)
Gly Gly Gly Glu (GGGE, SEQ ID NO: 896)
Gln Gln Gln Gly (QQQG, SEQ ID NO: 897)
Gln Gln Gln Gln Gly (QQQQG, SEQ ID NO: 898)
Ser Ser Ser Gly (SSSG, SEQ ID NO: 899)
Ser Ser Ser Ser Gly (SSSSG, SEQ ID NO: 900)

wherein n is an integer of 1 or larger.

However, the length and sequence of the peptide linker can be appropriately selected by those skilled in the art according to the purpose. The presence of a linker (such as the GS linker) in the hinge region between the Fab and Fc may result in heterogeneity in the disulphide bond formation between HC (heavy-chain constant region) and LC (light-chain constant region). In some embodiments, a fusion protein of the invention is a homodimer of a light chain and heavy chain. The heavy chain comprises a linker such as a cleavable linker (“L1”, e.g., SEQ ID NO: 873) introduced into the elbow hinge region between the heavy-chain variable region and Constant Region 1 (“C1”, e.g., SEQ ID NO: 901). A single-chain ligand (such as IL-12 or IL-22) may be attached to the C-terminus of Fc domain via a linker such as the GS linker (“L4”, e.g., SEQ ID NO: 903); alternatively, the linker may be a cleavable linker (“L3”, e.g., SEQ ID NO: 879).

This type of fusion protein may be called “C1” variant. In some embodiments, the variant is Ab1-L1-C1-L4-IL12 (Bivalent IL-12 fusion Ab1) which is a homodimer comprising a light chain of SEQ ID NO: 876 and a heavy chain of SEQ ID NO: 885. To promote homogeneity, improved forms (further variants) may be generated as follows.

In some embodiments, a “C2” variant is used. The heavy chain of this variant may comprise a linker such as a cleavable linker (“L1”, e.g., SEQ ID NO: 873) introduced into the elbow hinge region between the heavy-chain variable region and Constant Region 2 (“C2”, e.g., SEQ ID NO: 905). In the Constant Region 2, a non-limiting example of the positional shift of a linker (e.g., the GS linker (GGGGSGGGGS (SEQ ID NO: 141)₂) present in the hinge region) is shown below:
from [GGGGSGGGGSEPKSCDKTHTCPPCP] (SEQ ID NO: 937)
to [EPKSCGGGGSGGGGSDKTHTCPPCP] (SEQ ID NO: 935) (the initial residue (E) is at position 216 (EU numbering)). The shifted position of the linker can be appropriately selected or designed by those skilled in the art according to the purpose, i.e., to promote homogeneity. The positional shift of the linker can promote or facilitate disulphide (cysteine-cysteine (Cys-Cys)) bond formation between Cys at position 220 (C220) (EU numbering) of the heavy chain and Cys at position 214 (C214) (EU numbering) of the light chain. A single-chain ligand (such as IL-12 or IL-22) may be attached to the C-terminus of Fc domain via a linker such as the GS linker (“L4”, e.g., SEQ ID NO: 903); alternatively, the linker may be a cleavable linker (“L3”, e.g., SEQ ID NO: 879).

In some embodiments, the variant is Ab1-L1-C2-L4-IL12 which is a homodimer comprising a light chain of SEQ ID NO: 876 and a heavy chain of SEQ ID NO: 904.

In some embodiments, a “C3” variant is used. In this variant, the light chain may comprise C214S (EU numbering) modification and the heavy chain may comprise C220S (EU numbering) modification which result in no disulphide bond formation between the heavy chain and light chain, i.e., between position 220 (EU numbering) of the heavy chain and position 214 (EU numbering) of the light chain. The heavy chain of this variant may comprise a linker such as a cleavable linker (“L1”, e.g., SEQ ID NO: 873) introduced into the elbow hinge region between the heavy-chain variable region and Constant Region 3 (“C3”, e.g., SEQ ID NO: 908). A single-chain ligand (such as IL-12 and IL-22) may be attached to the C-terminus of Fc domain via a linker such as the GS linker (“L4”, e.g., SEQ ID NO: 903); alternatively, the linker may be a cleavable linker (“L3”, e.g., SEQ ID NO: 879).

In some embodiments, the variant is Ab1-L1-C3-L4-IL12 which is a homodimer comprising a light chain of SEQ ID NO: 906 and heavy chain of SEQ ID NO: 907.

In some embodiments, a “C4” variant is used. In this variant, the light chain may not comprise the above-mentioned modification(s), while the heavy chain may comprise S131C (EU numbering) and C220S (EU numbering) modifications which result in disulphide bond formation between the heavy chain and light chain, i.e., between Cys at position 131 (C131) (EU numbering) of the heavy chain and Cys at position 214 (C214) (EU numbering) of the light chain. The heavy chain of this variant may comprise a linker such as a cleavable linker (“L1”, e.g., SEQ ID NO: 873) introduced into the elbow hinge region between the heavy-chain variable region and Constant Region 4 (“C4”, e.g., SEQ ID NO: 910). A single-chain ligand (such as IL-12 or IL-22) may be attached to the C-terminus of Fc domain via a linker such as the GS linker (L4, e.g., SEQ ID NO: 903); alternatively, the linker may be a cleavable linker (“L3”, e.g., SEQ ID NO: 879).

In some embodiments, the variant is Ab1-L1-C4-L4-IL12 which is a homodimer comprising a light chain of SEQ ID NO: 876 and a heavy chain of SEQ ID NO: 909.

In some embodiments, a “C5” variant is used. The heavy chain of this variant may comprise a linker such as a cleavable linker (“L1”, e.g., SEQ ID NO: 873) introduced into the elbow hinge region between the heavy-chain variable region and Constant Region 5 (“C5”, e.g., SEQ ID NO: 932). In the Constant Region 5, a non-limiting example of the positional shift of a linker (e.g., the GS linker (GGGGSGGGGS (SEQ ID NO: 141)₂) present in the hinge region) is shown below:
from [GGGGSGGGGSEPKSCDKTHTCPPCP] (SEQ ID NO: 937)
to [EPKSCDKTHTGGGGSGGGGSCPPCP] (SEQ ID NO: 938) (the initial residue (E) is at position 216 (EU numbering)). The shifted position of the linker can be appropriately selected or designed by those skilled in the art according to the purpose, i.e., to promote homogeneity. The positional shift of the linker can promote or facilitate disulphide (cysteine-cysteine (Cys-Cys)) bond formation between Cys at position 220 (C220) (EU numbering) of the heavy chain and Cys at position 214 (C214) (EU numbering) of the light chain. A single-chain ligand (such as IL-12 or IL-22) may be attached to the C-terminus of Fc domain via a linker such as the GS linker (“L5”, e.g., SEQ ID NO: 927).

In one embodiment, the fusion protein is a bivalent ligand-binding fusion protein which comprises two sets (e.g., two identical sets) of the ligand-binding domain, the ligand moiety, the cleavable peptide linker, the constant (or Fc) region, and the non-cleavable peptide linker. When the fusion protein comprises two Fc regions, the regions dimerize with each other to form a ligand-binding moiety. In this case, in some embodiments, the fusion protein may be an IgG-type protein, e.g., IgG-type antibody, comprising two Fc regions that dimerize.

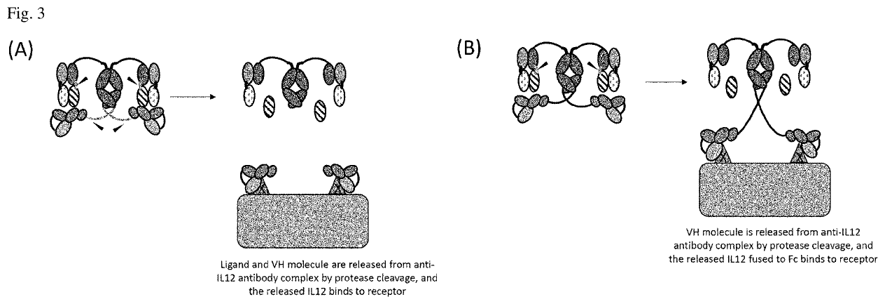

In one embodiment of the present invention, the ligand moiety is released from the ligand-binding domain of the ligand-binding moiety by protease cleavage of the fusion protein. In this context, when the ligand moiety is connected with the C- or N-terminal region of the ligand binding moiety via a peptide linker having a protease cleavage site, the ligand moiety may be completely released from the fusion protein. Herein, this type of fusion protein is referred to as “release type” (see e.g., FIG. 3A). On the other hand, when the ligand moiety is connected with the C-terminal region of the ligand binding moiety via a peptide linker having no protease cleavage site, the ligand moiety may be released from the ligand-binding domain while remaining fused to the C-terminal region of the ligand binding moiety via the peptide linker. Herein, this type of fusion protein is referred to as “fusion type” (see e.g., FIG. 3B).

A method for detecting release of the ligand moiety or molecule from the ligand-binding domain by cleavage of the protease cleavage site(s) includes a method of detecting the ligand using, for example, an antibody for ligand detection that recognizes the ligand. When the ligand binding moiety/molecule is an antibody fragment, the antibody for ligand detection preferably binds to the same epitope as that for the ligand binding domain. The ligand detected using the antibody for ligand detection can be confirmed by a well-known method such as FACS, an ELISA format, a BIACORE method using ALPHA (amplified luminescent proximity homogeneous assay) screening or surface plasmon resonance (SPR) phenomena, or BLI (bio-layer interferometry) (Octet) (Proc. Natl. Acad. Sci. USA (2006) 103 (11), 4005-4010).

In the case of detecting the release of the ligand using, for example, Octet, the antibody for ligand detection that recognizes the ligand is biotinylated and contacted with a biosensor. Then, binding to the ligand in a sample can be measured to detect the release of the ligand. Specifically, the amount of the ligand is measured in a sample containing the ligand binding molecule before protease treatment or after protease treatment and the ligand, using the antibody for ligand detection. The amount of the ligand detected in the sample can be compared between before and after protease treatment to detect the release of the ligand. Alternatively, the amount of the ligand is measured in a sample containing protease, the ligand binding molecule, and the ligand and a sample containing the ligand binding molecule and the ligand without containing protease, using the antibody for ligand detection. The amount of the ligand detected in the sample can be compared between the presence and absence of protease to detect the release of the ligand. More specifically, the release of the ligand can be detected by a method described in Examples of the present application. When the ligand binding molecule is fused with the ligand to form a fusion protein, the amount of the ligand is measured in a sample containing the fusion protein before protease treatment or after protease treatment, using the antibody for ligand detection. The amount of the ligand detected in the sample can be compared between before and after protease treatment to detect the release of the ligand. Alternatively, the amount of the ligand is measured in a sample containing protease and the fusion protein and a sample containing the fusion protein without containing protease, using the antibody for ligand detection. The amount of the ligand detected in the sample can be compared between the presence and absence of protease to detect the release of the ligand. More specifically, the release of the ligand can be detected by a method described in Examples of the present application.

In an embodiment in which the physiological activity of the ligand is attenuated upon binding to the ligand-binding domain, the release from the ligand-binding molecule can be detected by a method of measuring the physiological activity of the ligand in a sample. Specifically, the physiological activity of the ligand can be measured in a sample containing the ligand-binding molecule before protease treatment of after protease treatment and the ligand and compared between before and after protease treatment to detect the release of the ligand. Alternatively, the physiological activity of the ligand can be measured in a sample containing protease, the ligand-binding molecule, and the ligand and a sample containing the ligand-binding molecule and the ligand without containing protease and compared between these samples to detect the release of the ligand. When the ligand-binding molecule is fused with the ligand to form a fusion protein, the physiological activity of the ligand can be measured in a sample containing the fusion protein before protease treatment or after protease treatment and compared between before and after protease treatment to detect the release of the ligand. Alternatively, the physiological activity of the ligand can be measured in a sample containing protease and the fusion protein and a sample containing the fusion protein without containing protease and compared between these samples to detect the release of the ligand.

In some embodiments, the fusion protein comprises a protease cleavage site(s) comprising a protease cleavage sequence and is cleavable by a protease. In certain embodiments where the fusion protein of the present invention has a plurality of protease cleavage sites, those protease cleavage sites may have the same protease cleavage sequence or different protease cleavage sequences. When the protease cleavage sites have different protease cleavage sequences, those different protease cleavage sequences may be cleaved by the same protease or different proteases. In some embodiments of the present invention, the protease cleavage sites(s) may also comprise one or more amino acid residues at one or both ends of the protease cleavage sequence as long as those residues do not inhibit recognition and cleavage of the protease cleavage sequence by the protease.

### Protease

In the present specification, the term “protease” refers to an enzyme such as endopeptidase or exopeptidase which hydrolyses a peptide bond, and typically refers to endopeptidase. The protease used in the present invention is limited only by its capability of cleaving a protease cleavage sequence and is not limited to any particular type of protease. In some embodiments, target tissue specific protease is used. The target tissue specific protease can refer to, for example, any of
(1) protease that is expressed at a higher level in the target tissue than in normal tissues, (2) protease that has higher activity in the target tissue than in normal tissues,
(3) protease that is expressed at a higher level in the target cells than in normal cells, and
(4) protease that has higher activity in the target cells than in normal cells. In a more specific embodiment, a cancer tissue specific protease or an inflammatory tissue specific protease is used.

In the present specification, the term “target tissue” means a tissue containing at least one target cell. In some embodiments of the present invention, the target tissue is a cancer tissue. In some embodiments of the present invention, the target tissue is an inflammatory tissue.

The term “cancer tissue” means a tissue containing at least one cancer cell. Thus, considering that, for example, the cancer tissue contains cancer cells and vascular vessels, every cell type that contributes to the formation of tumour mass containing cancer cells and endothelial cells is included in the scope of the present invention. In the present specification, the tumour mass refers to a foci of tumour tissue. The term “tumour” is generally used to mean benign neoplasm or malignant neoplasm.

In the present specification, examples of the “inflammatory tissue” include the following:
a joint tissue in rheumatoid arthritis or osteoarthritis,
a lung (alveolus) tissue in bronchial asthma or Chronic obstructive pulmonary disease (COPD),
a digestive organ tissue in inflammatory bowel disease, Crohn's disease, or ulcerative colitis,
a fibrotic tissue in fibrosis in the liver, the kidney, or the lung,
a tissue under rejection of organ transplantation,
a vascular vessel or heart (cardiac muscle) tissue in arteriosclerosis or heart failure,
a visceral fat tissue in metabolic syndrome,
a skin tissue in atopic dermatitis and other dermatitis,
a spinal nerve tissue in disk herniation or chronic lumbago, and any tissue that is infiltrated with immune cells.

Specifically expressed or specifically activated protease, or protease considered to be related to the disease condition of a target tissue (target tissue specific protease) is known for some types of target tissues. For example, International Publication Nos. WO2013/128194, WO2010/081173, and WO2009/025846 disclose protease specifically expressed in a cancer tissue. Also, J Inflamm (Lond). 2010; 7: 45, Nat Rev Immunol. 2006 July; 6 (7): 541-50, Nat Rev Drug Discov. 2014 December; 13 (12): 904-27, Respir Res. 2016 Mar. 4; 17: 23, Dis Model Mech. 2014 February; 7 (2): 193-203, and Biochim Biophys Acta. 2012 January; 1824 (1): 133-45 disclose protease considered to be related to inflammation.

In addition to the protease specifically expressed in a target tissue, there also exists protease specifically activated in a target tissue. For example, protease may be expressed in an inactive form and then converted to an active form. Many tissues contain a substance inhibiting active protease and control the activity by the process of activation and the presence of the inhibitor (Nat Rev Cancer. 2003 July; 3 (7): 489-501). In a target tissue, the active protease may be specifically activated by escaping inhibition. The active protease can be measured by use of a method using an antibody recognizing the active protease (PNAS 2013 Jan. 2; 110 (1): 93-98) or a method of fluorescently labelling a peptide recognizable by protease so that the fluorescence is quenched before cleavage, but emitted after cleavage (Nat Rev Drug Discov. 2010 September; 9 (9): 690-701. doi: 10.1038/nrd3053).

From one viewpoint, the term “target tissue specific protease” can refer to any of
(i) protease that is expressed at a higher level in the target tissue than in normal tissues,
(ii) protease that has higher activity in the target tissue than in normal tissues,
(iii) protease that is expressed at a higher level in the target cells than in normal cells, and
(iv) protease that has higher activity in the target cells than in normal cells.

Specific examples of the protease include, but are not limited to, cysteine protease (including cathepsin families B, L, S, etc.), aspartyl protease (cathepsins D, E, K, O, etc.), serine protease (including matriptase (including MT-SP1), cathepsins A and G, thrombin, plasmin, urokinase-type plasminogen activator (uPA), tissue plasminogen activator (tPA), elastase, proteinase 3, thrombin, kallikrein, tryptase, and chymase), metalloproteinase (metalloproteinase (MMP1-28) including both membrane-bound forms (MMP14-17 and MMP24-25) and secreted forms (MMP1-13, MMP18-23 and MMP26-28), A disintegrin and metalloproteinase (ADAM), A disintegrin and metallo-proteinase with thrombospondin motifs (ADAMTS), meprin (meprin alpha and meprin beta), CD10 (CALLA), prostate-specific antigen (PSA), legumain, TMPRSS3, TMPRSS4, human neutrophil elastase (HNE), beta secretase (BACE), fibroblast activation protein alpha (FAP), granzyme B, guanidinobenzoatase (GB), hepsin, neprilysin, NS3/4A, HCV-NS3/4, calpain, ADAMDEC1, renin, cathepsin C, cathepsin V/L2, cathepsin X/Z/P, cruzipain, otubain 2, kallikrein-related peptidases (KLKs (KLK3, KLK4, KLK5, KLK6, KLK7, KLK8, KLK10, KLK11, KLK13, and KLK14)), bone morphogenetic protein 1 (BMP-1), activated protein C, blood coagulation-related protease (Factor VIIa, Factor IXa, Factor Xa, Factor XIa, and Factor XIIa), HtrA1, lactoferrin, marapsin, PACE4, DESC1, dipeptidyl peptidase 4 (DPP-4), TMPRSS2, cathepsin F, cathepsin H, cathepsin L2, cathepsin 0, cathepsin S, granzyme A, Gepsin calpain 2, glutamate carboxypeptidase 2, AMSH-like proteases, AMSH, gamma secretase, antiplasmin cleaving enzyme (APCE), decysin 1, N-acetylated alpha-linked acidic dipeptidase-like 1 (NAALADL1), and furin.

From another viewpoint, the target tissue specific protease can refer to cancer tissue specific protease or inflammatory tissue specific protease.

Examples of the cancer tissue specific protease include protease specifically expressed in a cancer tissue disclosed in International Publication Nos. WO2013/128194, WO2010/081173, and WO2009/025846.

As for the type of the cancer tissue specific protease, the protease having higher expression specificity in the cancer tissue to be treated is more effective for reducing adverse reactions. Preferable cancer tissue specific protease has a concentration in the cancer tissue at least 5 times, more preferably at least 10 times, further preferably at least 100 times, particularly preferably at least 500 times, most preferably at least 1000 times higher than its concentration in normal tissues. Also, preferable cancer tissue specific protease has activity in the cancer tissue at least 2 times, more preferably at least 3 times, at least 4 times, at least 5 times, or at least 10 times, further preferably at least 100 times, particularly preferably at least 500 times, most preferably at least 1000 times higher than its activity in normal tissues.

The cancer tissue specific protease may be in a form bound with a cancer cell membrane or may be in a form secreted extracellularly without being bound with a cell membrane. When the cancer tissue specific protease is not bound with a cancer cell membrane, it is preferred that the cancer tissue specific protease should exist within or in the vicinity of the cancer tissue. In the present specification, the “vicinity of the cancer tissue” means to fall within the scope of location where the protease cleavage sequence specific for the cancer tissue is cleaved so that the effect of reducing the ligand-binding activity is exerted.

From an alternative viewpoint, cancer tissue specific protease is any of
(i) protease that is expressed at a higher level in the cancer tissue than in normal tissues,
(ii) protease that has higher activity in the cancer tissue than in normal tissues,
(iii) protease that is expressed at a higher level in the cancer cells than in normal cells, and
(iv) protease that has higher activity in the cancer cells than in normal cells.

One type of cancer tissue specific protease may be used alone, or two or more types of cancer tissue specific proteases may be combined. The number of types of the cancer tissue specific protease can be appropriately set by those skilled in the art in consideration of the cancer type to be treated.

From these viewpoints, the cancer tissue specific protease is preferably serine protease or metalloproteinase, more preferably matriptase (including MT-SP1), urokinase-type plasminogen activator (uPA), or metalloproteinase, further preferably MT-SP1, uPA, MMP-2, or MMP-9, among the proteases listed above, particular preferably MMP-2, or MMP-9, among the proteases listed above.

As for the type of inflammatory tissue specific protease, the protease having higher expression specificity in the inflammatory tissue to be treated is more effective for reducing adverse reactions. Preferable inflammatory tissue specific protease has a concentration in the inflammatory tissue at least 5 times, more preferably at least 10 times, further preferably at least 100 times, particularly preferably at least 500 times, most preferably at least 1000 times higher than its concentration in normal tissues. Also, preferable inflammatory tissue specific protease has activity in the inflammatory tissues at least 2 times, more preferably at least 3 times, at least 4 times, at least 5 times, or at least 10 times, further preferably at least 100 times, particularly preferably at least 500 times, most preferably at least 1000 times higher than its activity in normal tissues.

The inflammatory tissue specific protease may be in a form bound with an inflammatory cell membrane or may be in a form secreted extracellularly without being bound with a cell membrane. When the inflammatory tissue specific protease is not bound with an inflammatory cell membrane, it is preferred that the inflammatory tissue specific protease should exist within or in the vicinity of the inflammatory tissue. In the present specification, the “vicinity of the inflammatory tissue” means to fall within the scope of location where the protease cleavage sequence specific for the inflammatory tissue is cleaved so that the effect of reducing the ligand binding activity is exerted.

From an alternative viewpoint, inflammatory tissue specific protease is any of
(i) protease that is expressed at a higher level in the inflammatory tissue than in normal tissues,
(ii) protease that has higher activity in the inflammatory tissue than in normal tissues,
(iii) protease that is expressed at a higher level in the inflammatory cells than in normal cells, and
(iv) protease that has higher activity in the inflammatory cells than in normal cells. One type of inflammatory tissue specific protease may be used alone, or two or more types of inflammatory tissue specific proteases may be combined. The number of types of the inflammatory tissue specific protease can be appropriately set by those skilled in the art in consideration of the pathological condition to be treated.

From these viewpoints, the inflammatory tissue specific protease is preferably metalloproteinase among the proteases listed above. The metalloproteinase is more preferably ADAMTS5, MMP-1, MMP-2, MMP-3, MMP-7, MMP-9, MMP11, or MMP-13.

### Protease Cleavage Sequence

The protease cleavage sequence is a particular amino acid sequence that is specifically recognized by target tissue specific protease when the polypeptide is hydrolysed by the target tissue specific protease in an aqueous solution. The protease cleavage sequence is preferably an amino acid sequence that is hydrolysed with high specificity by target tissue specific protease more specifically expressed in the target tissue or cells to be treated or more specifically activated in the target tissue/cells to be treated, from the viewpoint of reduction in adverse reactions.

Specific examples of the protease cleavage sequence include target sequences that are specifically hydrolysed by the above-listed protease specifically expressed in a cancer tissue disclosed in International Publication Nos. WO2013/128194, WO2010/081173, and WO2009/025846, the inflammatory tissue specific protease, and the like. A sequence artificially altered by, for example, appropriately introducing an amino acid mutation to a target sequence that is specifically hydrolysed by known protease can also be used. Alternatively, a protease cleavage sequence identified by a method known to those skilled in the art as described in Nature Biotechnology 19, 661-667 (2001) may be used.

Furthermore, a naturally occurring protease cleavage sequence may be used. For example, TGF beta is converted to a latent form by protease cleavage. Likewise, a protease cleavage sequence in a protein that changes its molecular form by protease cleavage can also be used.

Examples of the protease cleavage sequence that can be used include, but are not limited to, sequences disclosed in WO2015/116933, WO2015/048329, WO2016/118629, WO2016/179257, WO2016/179285, WO2016/179335, WO2016/179003, WO2016/046778, WO2016/014974, U.S. Patent Publication No. US2016/0289324, U.S. Patent Publication No. US2016/0311903, PNAS (2000) 97: 7754-7759, Biochemical Journal (2010) 426: 219-228, and Beilstein J Nanotechnol. (2016) 7: 364-373.

The protease cleavage sequence is more preferably an amino acid sequence that is specifically hydrolysed by suitable target tissue specific protease as mentioned above. The amino acid sequence that is specifically hydrolysed by target tissue specific protease is preferably any of the following amino acid sequences: LSGRSDNH (SEQ ID NO: 2, cleavable by MT-SP1 or uPA), PLGLAG (SEQ ID NO: 3, cleavable by MMP-2 or MMP-9), and VPLSLTMG (SEQ ID NO: 4, cleavable by MMP-7).

Any of the following sequences can also be used as the protease cleavage sequence:

TSTSGRSANPRG (SEQ ID NO: 5, cleavable by MT-SP1 or uPA),
ISSGLLSGRSDNH (SEQ ID NO: 6, cleavable by MT-SP1 or uPA),
AVGLLAPPGGLSGRSDNH (SEQ ID NO: 7, cleavable by MT-SP1 or uPA),
GAGVPMSMRGGAG (SEQ ID NO: 8, cleavable by MMP-1),
GAGIPVSLRSGAG (SEQ ID NO: 9, cleavable by MMP-2),
GPLGIAGQ (SEQ ID NO: 10, cleavable by MMP-2),
GGPLGMLSQS (SEQ ID NO: 11, cleavable by MMP-2),
PLGLWA (SEQ ID NO: 12, cleavable by MMP-2),
GAGRPFSMIMGAG (SEQ ID NO: 13, cleavable by MMP-3),
GAGVPLSLTMGAG (SEQ ID NO: 14, cleavable by MMP-7),
GAGVPLSLYSGAG (SEQ ID NO: 15, cleavable by MMP-9),
AANLRN (SEQ ID NO: 16, cleavable by MMP-11),
AQAYVK (SEQ ID NO: 17, cleavable by MMP-11),
AANYMR (SEQ ID NO: 18, cleavable by MMP-11),
AAALTR (SEQ ID NO: 19, cleavable by MMP-11),
AQNLMR (SEQ ID NO: 20, cleavable by MMP-11),
AANYTK (SEQ ID NO: 21, cleavable by MMP-11),
GAGPQGLAGQRGIVAG (SEQ ID NO: 22, cleavable by MMP-13),
PRFKIIGG (SEQ ID NO: 23, cleavable by pro-urokinase),
PRFRIIGG (SEQ ID NO: 24, cleavable by pro-urokinase),
GAGSGRSAG (SEQ ID NO: 25, cleavable by uPA),
SGRSA (SEQ ID NO: 26, cleavable by uPA),
GSGRSA (SEQ ID NO: 27, cleavable by uPA),
SGKSA (SEQ ID NO: 28, cleavable by uPA),
SGRSS (SEQ ID NO: 29, cleavable by uPA),
SGRRA (SEQ ID NO: 30, cleavable by uPA),
SGRNA (SEQ ID NO: 31, cleavable by uPA),
SGRKA (SEQ ID NO: 32, cleavable by uPA),
QRGRSA (SEQ ID NO: 33, cleavable by tPA),
GAGSLLKSRMVPNFNAG (SEQ ID NO: 34, cleavable by cathepsin B)
TQGAAA (SEQ ID NO: 35, cleavable by cathepsin B),
GAAAAA (SEQ ID NO: 36, cleavable by cathepsin B),
GAGAAG (SEQ ID NO: 37, cleavable by cathepsin B),
AAAAAG (SEQ ID NO: 38, cleavable by cathepsin B),
LCGAAI (SEQ ID NO: 39, cleavable by cathepsin B),
FAQALG (SEQ ID NO: 40, cleavable by cathepsin B),
LLQANP (SEQ ID NO: 41, cleavable by cathepsin B),
LAAANP (SEQ ID NO: 42, cleavable by cathepsin B),
LYGAQF (SEQ ID NO: 43, cleavable by cathepsin B),
LSQAQG (SEQ ID NO: 44, cleavable by cathepsin B),
ASAASG (SEQ ID NO: 45, cleavable by cathepsin B),
FLGASL (SEQ ID NO: 46, cleavable by cathepsin B),
AYGATG (SEQ ID NO: 47, cleavable by cathepsin B),
LAQATG (SEQ ID NO: 48, cleavable by cathepsin B),
GAGSGVVIATVIVITAG (SEQ ID NO: 49, cleavable by cathepsin L),
APMAEGGG (SEQ ID NO: 50, cleavable by meprin alpha or meprin beta),
EAQGDKII (SEQ ID NO: 51, cleavable by meprin alpha or meprin beta),
LAFSDAGP (SEQ ID NO: 52, cleavable by meprin alpha or meprin beta),
YVADAPK (SEQ ID NO: 53, cleavable by meprin alpha or meprin beta),
RRRRR (SEQ ID NO: 54, cleavable by furin),
RRRRRR (SEQ ID NO: 55, cleavable by furin),
GQSSRHRRAL (SEQ ID NO: 56, cleavable by furin),
SSRHRRALD (SEQ ID NO: 57),
RKSSIIIRMRDVVL (SEQ ID NO: 58, cleavable by plasminogen),
SSSFDKGKYKKGDDA (SEQ ID NO: 59, cleavable by staphylokinase),
SSSFDKGKYKRGDDA (SEQ ID NO: 60, cleavable by staphylokinase),
IEGR (SEQ ID NO: 61, cleavable by Factor IXa),
IDGR (SEQ ID NO: 62, cleavable by Factor IXa),
GGSIDGR (SEQ ID NO: 63, cleavable by Factor IXa),
GPQGIAGQ (SEQ ID NO: 64, cleavable by collagenase),
GPQGLLGA (SEQ ID NO: 65, cleavable by collagenase),
GIAGQ (SEQ ID NO: 66, cleavable by collagenase),
GPLGIAG (SEQ ID NO: 67, cleavable by collagenase),
GPEGLRVG (SEQ ID NO: 68, cleavable by collagenase),
YGAGLGVV (SEQ ID NO: 69, cleavable by collagenase),
AGLGVVER (SEQ ID NO: 70, cleavable by collagenase),
AGLGISST (SEQ ID NO: 71, cleavable by collagenase),
EPQALAMS (SEQ ID NO: 72, cleavable by collagenase),
QALAMSAI (SEQ ID NO: 73, cleavable by collagenase),
AAYHLVSQ (SEQ ID NO: 74, cleavable by collagenase),
MDAFLESS (SEQ ID NO: 75, cleavable by collagenase),
ESLPVVAV (SEQ ID NO: 76, cleavable by collagenase),
SAPAVESE (SEQ ID NO: 77, cleavable by collagenase),
DVAQFVLT (SEQ ID NO: 78, cleavable by collagenase),
VAQFVLTE (SEQ ID NO: 79, cleavable by collagenase),
AQFVLTEG (SEQ ID NO: 80, cleavable by collagenase),
PVQPIGPQ (SEQ ID NO: 81, cleavable by collagenase),
LVPRGS (SEQ ID NO: 82, cleavable by thrombin),
TSTSGRSANPRG (SEQ ID NO: 83),
TSTSGRSANPRG (SEQ ID NO: 84),
TSGSGRSANARG (SEQ ID NO: 85)
TSQSGRSANQRG (SEQ ID NO: 86)
TSPSGRSAYPRG (SEQ ID NO: 87)
TSGSGRSATPRG (SEQ ID NO: 88)
TSQSGRSATPRG (SEQ ID NO: 89)
TSASGRSATPRG (SEQ ID NO: 90)
TSYSGRSAVPRG (SEQ ID NO: 91)
TSYSGRSANFRG (SEQ ID NO: 92)
TSSSGRSATPRG (SEQ ID NO: 93)
TSTTGRSASPRG (SEQ ID NO: 94)
TSTSGRSANPRG (SEQ ID NO: 95)

The sequences shown in Table 1 may also be used as protease cleavage sequences.

TABLE 1
Protease cleavage sequences (cleavable by uPA and MT-SP1)

| SEQ ID NO | Cleavage sequence | SEQ ID NO | Cleavage sequence |
|---|---|---|---|
| 160 | TSASGRSANPRG | 192 | TSTSQRSANPRG |
| 161 | TSESGRSANPRG | 193 | TSTSRRSANPRG |
| 162 | TSFSGRSANPRG | 194 | TSTSTRSANPRG |
| 163 | TSGSGRSANPRG | 195 | TSTSVRSANPRG |
| 164 | TSHSGRSANPRG | 196 | TSTSWRSANPRG |
| 165 | TSKSGRSANPRG | 197 | TSTSYRSANPRG |
| 166 | TSMSGRSANPRG | 198 | TSTSGRAANPRG |
| 167 | TSNSGRSANPRG | 199 | TSTSGRDANPRG |
| 168 | TSPSGRSANPRG | 200 | TSTSGREANPRG |
| 169 | TSQSGRSANPRG | 201 | TSTSGRGANPRG |
| 170 | TSWSGRSANPRG | 202 | TSTSGRHANPRG |
| 171 | TSYSGRSANPRG | 203 | TSTSGRIANPRG |
| 172 | TSTAGRSANPRG | 204 | TSTSGRKANPRG |
| 173 | TSTDGRSANPRG | 205 | TSTSGRLANPRG |
| 174 | TSTEGRSANPRG | 206 | TSTSGRMANPRG |
| 175 | TSTFGRSANPRG | 207 | TSTSGRNANPRG |
| 176 | TSTLGRSANPRG | 208 | TSTSGRPANPRG |
| 177 | TSTMGRSANPRG | 209 | TSTSGRGANPRG |
| 178 | TSTPGRSANPRG | 210 | TSTSGRRANPRG |
| 179 | TSTQGRSANPRG | 211 | TSTSGRTANPRG |
| 180 | TSTVGRSANPRG | 212 | TSTSGRVANPRG |
| 181 | TSTWGRSANPRG | 213 | TSTSGRWANPRG |
| 182 | TSTSARSANPRG | 214 | TSTSGRYANPRG |
| 183 | TSTSERSANPRG | 215 | TSTSGRSENPRG |
| 184 | TSTSFRSANPRG | 216 | TSTSGRSFNPRG |
| 185 | TSTSHRSANPRG | 217 | TSTSGRSKNPRG |
| 186 | TSTSIRSANPRG | 218 | TSTSGRSMNPRG |
| 187 | TSTSKRSANPRG | 219 | TSTSGRSNNPRG |
| 188 | TSTSLRSANPRG | 220 | TSTSGRSPNPRG |
| 189 | TSTSMRSANPRG | 221 | TSTSGRSQNPRG |
| 190 | TSTSNRSANPRG | 222 | TSTSGRSRNPRG |
| 191 | TSTSPRSANPRG | 223 | TSTSGRSSNPRG |
| 224 | TSTSGRSWNPRG | 257 | TSTYGRSANPRG |
| 225 | TSTSGRSYNPRG | 258 | TSTSDRSANPRG |
| 226 | TSTSGRSAAPRG | 259 | TSTSSRSANPRG |
| 227 | TSTSGRSADPRG | 260 | TSTSGRPANPRG |
| 228 | TSTSGRSAEPRG | 261 | TSTSGRSDNPRG |
| 229 | TSTSGRSAFPRG | 262 | TSTSGRSHNPRG |
| 230 | TSTSGRSAGPRG | 263 | TSTSGRSINPRG |
| 231 | TSTSGRSAKPRG | 264 | TSTSGRSLNPRG |
| 232 | TSTSGRSALPRG | 265 | TSTSGRSTNPRG |
| 233 | TSTSGRSAMPRG | 266 | TSTSGRSVNPRG |
| 234 | TSTSGRSAPPRG | 267 | TSTSGRSAHPRG |
| 235 | TSTSGRSAQPRG | 268 | TSTSGRSAIPRG |
| 236 | TSTSGRSAVPRG | 269 | TSTSGRSARPRG |
| 237 | TSTSGRSAWPRG | 270 | TSTSGRSASPRG |
| 238 | TSTSGRSAYPRG | 271 | TSTSGRSATPRG |
| 239 | TSTSGRSANARG | 272 | TSTSGRSANHRG |
| 240 | TSTSGRSANDRG | 273 | TSTSGRSANLRG |
| 241 | TSTSGRSANERG | 274 | TSTSGRSANMRG |
| 242 | TSTSGRSANFRG | 275 | TSTSGRSANRRG |
| 243 | TSTSGRSANGRG | 276 | TSTSGRSANVRG |
| 244 | TSTSGRSANIRG | 277 | TSTSGRSANYRG |
| 245 | TSTSGRSANKRG | 278 | TSGSGRSAVPRG |
| 246 | TSTSGRSANNRG | 279 | TSGSGRSAYPRG |
| 247 | TSTSGRSANQRG | 280 | TSGSGRSANQRG |
| 248 | TSTSGRSANSRG | 281 | TSGSGRSANIRG |
| 249 | TSTSGRSANTRG | 282 | TSGSGRSANFRG |
| 250 | TSTSGRSANWRG | 283 | TSGSGRSANSRG |
| 251 | TSDSGRSANPRG | 284 | TSQSGRSAVPRG |
| 252 | TSISGRSANPRG | 285 | TSQSGRSAYPRG |
| 253 | TSSSGRSANPRG | 286 | TSQSGRSANARG |
| 254 | TSTHGRSANPRG | 287 | TSQSGRSANIRG |
| 255 | TSTKGRSANPRG | 288 | TSQSGRSANFRG |
| 256 | TSTTGRSANPRG | 289 | TSQSGRSANSRG |
| 290 | TSPSGRSAVPRG | 323 | TSTSGRSAEFRG |
| 291 | TSPSGRSANQRG | 324 | TSTSGRSAESRG |
| 292 | TSPSGRSANARG | 325 | TSGTGRSANPRG |
| 293 | TSPSGRSANIRG | 326 | TSGKGRSANPRG |
| 294 | TSPSGRSANFRG | 327 | TSGSGRSAIPRG |
| 295 | TSPSGRSANSRG | 328 | TSGSGRSASPRG |
| 296 | TSASGRSAVPRG | 329 | TSGSGRSAHPRG |
| 297 | TSASGRSAYPRG | 330 | TSGSGRSANYRG |
| 298 | TSASGRSANQRG | 331 | TSGSGRSANVRG |
| 299 | TSASGRSANARG | 332 | TSGSGRSANHRG |
| 300 | TSASGRSANIRG | 333 | TSQTGRSANPRG |
| 301 | TSASGRSANFRG | 334 | TSQKGRSANPRG |
| 302 | TSASGRSANSRG | 335 | TSQSGRSAIPRG |
| 303 | TSYSGRSENPRG | 336 | TSQSGRSASPRG |
| 304 | TSGSGRSENPRG | 337 | TSQSGRSAHPRG |
| 305 | TSQSGRSENPRG | 338 | TSQSGRSANYRG |
| 306 | TSPSGRSENPRG | 339 | TSQSGRSANVRG |
| 307 | TSASGRSENPRG | 340 | TSQSGRSANHRG |
| 308 | TSHSGRSENPRG | 341 | TSPTGRSANPRG |
| 309 | TSTSGRSENQRG | 342 | TSPKGRSANPRG |
| 310 | TSTSGRSENARG | 343 | TSPSGRSAIPRG |
| 311 | TSTSGRSENIRG | 344 | TSPSGRSATPRG |
| 312 | TSTSGRSENFRG | 345 | TSPSGRSASPRG |
| 313 | TSTSGRSENSRG | 346 | TSPSGRSAHPRG |
| 314 | TSYSGRSAEPRG | 347 | TSPSGRSANYRG |
| 315 | TSGSGRSAEPRG | 348 | TSPSGRSANVRG |
| 316 | TSQSGRSAEPRG | 349 | TSPSGRSANPRG |
| 317 | TSPSGRSAEPRG | 350 | TSATGRSANPRG |
| 318 | TSASGRSAPPRG | 351 | TSAKGRSANPRG |
| 319 | TSHSGRSAEPRG | 352 | TSASGRSAIPRG |
| 320 | TSTSGRSAEQRG | 353 | TSASGRSASPRG |
| 321 | TSTSGRSAEARG | 354 | TSASGRSAHPRG |
| 322 | TSTSGRSAEIRG | 355 | TSASGRSANYRG |
| 356 | TSASGRSANVRG | 389 | TSISGRSANVRG |
| 357 | TSASGRSANHRG | 390 | TSISGRSANHRG |
| 358 | TSYTGRSANPRG | 391 | TSTTGRSAVPRG |
| 359 | TSYKGRSANPRG | 392 | TSTTGRSAIPRG |
| 360 | TSYSGRSAIPRG | 393 | TSTTGRSATPRG |
| 361 | TSYSGRSATPRG | 394 | TSTTGRSAHPRG |
| 362 | TSYSGRSASPRG | 395 | TSTTGRSANARG |
| 363 | TSYSGRSAHPRG | 396 | TSTTGRSANFRG |
| 364 | TSYSGRSANARG | 397 | TSTTGRSANYRG |
| 365 | TSYSGRSANYRG | 398 | TSTTGRSANVRG |
| 366 | TSYSGRSANVRG | 399 | TSTTGRSANHRG |
| 367 | TSYSGRSANHRG | 400 | TSTKGRSAVPRG |
| 368 | TSSTGRSANPRG | 401 | TSTKGRSAIPRG |
| 369 | TSSKGRSANPRG | 402 | TSTKGRSATPRG |
| 370 | TSSSGRSAVPRG | 403 | TSTKGRSASPRG |
| 371 | TSSSGRSAIPRG | 404 | TSTKGRSAHPRG |
| 372 | TSSSGRSASPRG | 405 | TSTKGRSANARG |
| 373 | TSSSGRSAHPRG | 406 | TSTKGRSANFRG |
| 374 | TSSSGRSANARG | 407 | TSTKGRSANYRG |
| 375 | TSSSGRSANFRG | 408 | TSTKGRSANVRG |
| 376 | TSSSGRSANYRG | 409 | TSTKGRSANHRG |
| 377 | TSSSGRSANVRG | 410 | TSTSGRSAVYRG |
| 378 | TSSSGRSANHRG | 411 | TSTSGRSAVVRG |
| 379 | TSITGRSANPRG | 412 | TSTSGRSAVHRG |
| 380 | TSIKGRSANPRG | 413 | TSTSGRSAIYRG |
| 381 | TSISGRSAVPRG | 414 | TSTSGRSAIVRG |
| 382 | TSISGRSAIPRG | 415 | TSTSGRSAIHRG |
| 383 | TSISGRSATPRG | 416 | TSTSGRSASYRG |
| 384 | TSISGRSASPRG | 417 | TSTSGRSASVRG |
| 385 | TSISGRSAHPRG | 418 | TSTSGRSASHRG |
| 386 | TSISGRSANARG | 419 | TSTSGRSAHYRG |
| 387 | TSISGRSANFRG | 420 | TSTSGRSAHVRG |
| 388 | TSISGRSANYRG | 421 | TSTSGRSAHHRG |
| 422 | TSPSGRSEVPRG | 455 | TSYSGRSAVHGG |
| 423 | TSPSGRSAEPRG | 456 | TSYTGRSAVYGG |
| 424 | TSPSGRSAGPRG | 457 | TSYTGRSAVVGG |
| 425 | TSASGRSENARG | 458 | TSYTGRSAVHGG |
| 426 | TSASGRSAEARG | 459 | ASGRSANP |
| 427 | TSASGRSAGARG | 460 | ESGRSANP |
| 428 | TSGTGRSATPRG | 461 | FSGRSANP |
| 429 | TSGSGRSATYRG | 462 | GSGRSANP |
| 430 | TSGSGRSATVRG | 463 | HSGRSANP |
| 431 | TSGSGRSATHRG | 464 | KSGRSANP |
| 432 | TSGTGRSATYRG | 465 | MSGRSANP |
| 433 | TSGTGRSATVRG | 466 | NSGRSANP |
| 434 | TSGTGRSATHRG | 467 | PSGRSANP |
| 435 | TSGSGRSETPRG | 468 | QSGRSANP |
| 436 | TSGTGRSETPRG | 469 | WSGRSANP |
| 437 | TSGSGRSETYRG | 470 | YSGRSANP |
| 438 | TSGSGRSETVRG | 471 | TAGRSANP |
| 439 | TSGSGRSETHRG | 472 | TDGRSANP |
| 440 | TSYTGRSAVPRG | 473 | TEGRSANP |
| 441 | TSYSGRSAVYRG | 474 | TFGRSANP |
| 442 | TSYSGRSAVVRG | 475 | TLGRSANP |
| 443 | TSYSGRSAVHRG | 476 | TMGRSANP |
| 444 | TSYTGRSAVYRG | 477 | TPGRSANP |
| 445 | TSYTGRSAVVRG | 478 | TQGRSANP |
| 446 | TSYTGRSAVHRG | 479 | TVGRSANP |
| 447 | TSYSGRSEVPRG | 480 | TWGRSANP |
| 448 | TSYTGRSEVPRG | 481 | TSARSANP |
| 449 | TSYSGRSEVYRG | 482 | TSERSANP |
| 450 | TSYSGRSEVVRG | 483 | TSFRSAND |
| 451 | TSYSGRSEVHRG | 484 | TSHRSANP |
| 452 | TSYTGRSAVPGG | 485 | TSIRSANP |
| 453 | TSYSGRSAVYGG | 486 | TSKRSANP |
| 454 | TSYSGRSAVVGG | 487 | TSLRSANP |
| 488 | TSMRSANP | 521 | TSGRSRNP |
| 489 | TSNRSANP | 522 | TSGRSSNP |
| 490 | TSPRSANP | $53 | TSGRSWNP |
| 491 | TSQRSANP | 524 | TSGRSYNP |
| 492 | TSRRSANP | 525 | TSGRSAAP |
| 493 | TSTRSANP | 526 | TSGRSADP |
| 494 | TSVRSANP | 527 | TSGRSAEP |
| 495 | TSWRSANP | 528 | TSGRSAFP |
| 496 | TSYRSANP | 529 | TSGRSAGP |
| 497 | TSGRAANP | 530 | TSGRSAKP |
| 498 | TSGRDANP | 531 | TSGRSALP |
| 499 | TSGREANP | 532 | TSGRSAMP |
| 500 | TSGRGANP | 533 | TSGRSAPP |
| 501 | TSGRHANP | 534 | TSGRSAQP |
| 502 | TSGRIANP | 535 | TSGRSAVP |
| 503 | TSGRKANP | 536 | TSGRSAWP |
| 504 | TSGRLANP | 537 | TSGRSAYP |
| 505 | TSGRMANP | 538 | TSGRSANA |
| 506 | TSGRNANP | 539 | TSGRSAND |
| 507 | TSGRPANP | 540 | TSGRSANE |
| 508 | TSGRQANP | 541 | TSGRSANF |
| 509 | TSGRRANP | 542 | TSGRSANG |
| 510 | TSGRTANP | 543 | TSGRSANI |
| 511 | TSGRVANP | 544 | TSGRSANK |
| 512 | TSGRWANP | 545 | TSGRSANN |
| 513 | TSGRYANP | 546 | TSGRSANQ |
| 514 | TSGRSENP | 547 | TSGRSANS |
| 515 | TSGRSFNP | 548 | TSGRSANT |
| 516 | TSGRSKNP | 549 | TSGRSANW |
| 517 | TSGRSMNP | 550 | DSGRSANP |
| 518 | TSGRSNNP | 551 | ISGRSANP |
| 519 | TSGRSPNP | 552 | SSGRSANP |
| 520 | TSGRSQNP | 553 | THGRSANP |
| 554 | TKGRSANP | 587 | QSGRSANA |
| 555 | TTGRSANP | 588 | QSGRSANI |
| 556 | TYGRSANP | 589 | QSGRSANF |
| 557 | TSDRSANP | 590 | QSGRSANS |
| 558 | TSSRSANP | 591 | PSGRSAVP |
| 559 | TSGREANP | 592 | PSGRSAYP |
| 560 | TSGRSDNP | 593 | PSGRSANQ |
| 561 | TSGRSHNP | 594 | PSGRSANA |
| 562 | TSGRSINP | 595 | PSGRSANI |
| 563 | TSGRSLNP | 596 | PSGRSANF |
| 564 | TSGRSTNP | 597 | PSGRSANS |
| 565 | TSGRSVNP | 598 | ASGRSAVP |
| 566 | TSGRSAHP | 599 | ASGRSAYP |
| 567 | TSGRSAIP | 600 | ASGRSANQ |
| 568 | TSGRSARP | 601 | ASGRSANA |
| 569 | TSGRSASP | 602 | ASGRSANI |
| 570 | TSGRSATP | 603 | ASGRSANF |
| 571 | TSGRSANH | 604 | ASGRSANS |
| 572 | TSGRSANL | 605 | YSGRSENP |
| 573 | TSGRSANM | 606 | GSGRSENP |
| 574 | TSGRSANR | 607 | QSGRSENP |
| 575 | TSGRSANV | 608 | PSGRSENP |
| 576 | TSGRSANY | 609 | ASGRSENP |
| 577 | GSGRSAVP | 610 | HSGRSENP |
| 578 | GSGRSAYP | 611 | TSGRSENQ |
| 579 | GSGRSANQ | 612 | TSGRSENA |
| 580 | GSGRSANA | 613 | TSGRSENI |
| 581 | GSGRSANI | 614 | TSGRSENF |
| 582 | GSGRSANF | 615 | TSGRSENS |
| 583 | GSGRSANS | 616 | YSGRSAEP |
| 584 | QSGRSAVP | 617 | GSGRSAEP |
| 585 | QSGRSAYP | 618 | QSGRSAEP |
| 586 | QSGRSANQ | 619 | PSGRSAEP |
| 620 | ASGRSAEP | 653 | PSGRSANH |
| 621 | HSGRSAEP | 654 | ATGRSANP |
| 622 | TSGRSAEQ | 655 | AKGRSANP |
| 623 | TSGRSAEA | 656 | ASGRSAIP |
| 624 | TSGRSAEI | 657 | ASGRSATP |
| 625 | TSGRSAEF | 658 | ASGRSASP |
| 626 | TSGRSAES | 659 | ASGRSAHP |
| 627 | GTGRSANP | 660 | ASGRSANY |
| 628 | GKGRSANP | 661 | ASGRSANV |
| 629 | GSGRSAIP | 662 | ASGRSANH |
| 630 | GSGRSATP | 663 | YTGRSANP |
| 631 | GSGRSASP | 664 | YKGRSANP |
| 632 | GSGRSAHP | 665 | YSGRSAVP |
| 633 | GSGRSANY | 666 | YSGRSAIP |
| 634 | GSGRSANV | 667 | YSGRSATP |
| 635 | GSGRSANH | 668 | YSGRSASP |
| 636 | QTGRSANP | 669 | YSGRSAHP |
| 637 | QKGRSANP | 670 | YSGRSANA |
| 638 | QSGRSAIP | 671 | YSGRSANF |
| 639 | QSGRSATP | 672 | YSGRSANY |
| 640 | QSGRSASP | 673 | YSGRSANV |
| 641 | QSGRSAHP | 674 | YSGRSANH |
| 642 | QSGRSANY | 675 | STGRSANP |
| 643 | QSGRSANV | 676 | SKGRSANP |
| 644 | QSGRSANH | 677 | SSGRSAVP |
| 645 | PTGRSANP | 678 | SSGRSAIP |
| 646 | PKGRSANP | 679 | SSGRSATP |
| 647 | PSGRSAIP | 680 | SSGRSASP |
| 648 | PSGRSATP | 681 | SSGRSAHP |
| 649 | PSGRSASP | 682 | SSGRSANA |
| 650 | PSGRSAHP | 683 | SSGRSANF |
| 651 | PSGRSANY | 684 | SSGRSANY |
| 652 | PSGRSANV | 685 | SSGRSANV |
| 686 | SSGRSANH | 719 | TSGRSAVY |
| 687 | ITGRSANP | 720 | TSGRSAVV |
| 688 | IKGRSANP | 721 | TSGRSAVH |
| 689 | ISGRSAVP | 722 | TSGRSAIY |
| 690 | ISGRSAIP | 723 | TSGRSAIV |
| 691 | ISGRSATP | 724 | TSGRSAIH |
| 692 | ISGRSASP | 725 | TSGRSASY |
| 693 | ISGRSAHP | 726 | TSGRSASV |
| 694 | ISGRSANA | 727 | TSGRSASH |
| 695 | ISGRSANF | 728 | TSGRSAHY |
| 696 | ISGRSANY | 729 | TSGRSAHV |
| 697 | ISGRSANV | 730 | TSGRSAHH |
| 698 | ISGRSANH | 731 | PSGRSEVP |
| 699 | TTGRSAVP | 732 | PSGRSAEP |
| 700 | TTGRSAIP | 733 | PSGRSAGP |
| 701 | TTGRSATP | 734 | ASGRSENA |
| 702 | TTGRSASP | 735 | ASGRSAEA |
| 703 | TTGRSAHP | 736 | ASGRSAGA |
| 704 | TTGRSANA | 737 | GTGRSATP |
| 705 | TTGRSANF | 738 | GSGRSATY |
| 706 | TTGRSANY | 739 | GSGRSATV |
| 707 | TTGRSANV | 740 | GSGRSATH |
| 708 | TTGRSANH | 741 | GTGRSATY |
| 709 | TKGRSAVP | 742 | GTGRSATV |
| 710 | TKGRSAIP | 743 | GTGRSATH |
| 711 | TKGRSATP | 744 | GSGRSETP |
| 712 | TKGRSASP | 745 | GTGRSETP |
| 713 | TKGRSAHP | 746 | GSGRSETY |
| 714 | TKGRSANA | 747 | GSGRSETV |
| 715 | TKGRSANF | 748 | GSGRSETH |
| 716 | TKGRSANY | 749 | YTGRSAVP |
| 717 | TKGRSANV | 750 | YSGRSAVY |
| 718 | TKGRSANH | 751 | YSGRSAVV |
| 752 | YSGRSAVH | 785 | TSYTGRSAVPAG |
| 753 | YTGRSAVY | 786 | TSYTGRSAVYAG |
| 754 | YTGRSAVV | 787 | TSYTGRSAVVAG |
| 755 | YTGRSAVH | 788 | TSYTGRSAVHAG |
| 756 | YSGRSEVP | 789 | TSYSGRSAVPHG |
| 757 | YTGRSEVP | 790 | TSPSGRSANIHG |
| 758 | YSGRSEVY | 791 | TSPSGRSANFHG |
| 759 | YSGRSEVV | 792 | TSPTGRSANPHG |
| 760 | YSGRSEVH | 793 | TSPSGRSAIPHG |
| 761 | YTGRSAVP | 794 | TSYTGRSANPHG |
| 762 | YSGRSAVY | 795 | TSYSGRSAIPHG |
| 763 | YSGRSAVV | 796 | TSISGRSANYHG |
| 764 | YSGRSAVA | 797 | TSPSGRSAGPHG |
| 765 | YTGRSAVY | 798 | TSYTGRSAVPHG |
| 766 | YTGRSAVV | 799 | TSYTGRSAVYHG |
| 767 | YTGRSAVH | 800 | TSYTGRSAVVHG |
| 768 | TSTSGRSANPRG | 801 | TSYTGRSAVHHG |
| 769 | TSTSGRSANPAG | 802 | TSYSGRSAVPIG |
| 770 | TSTSGRSANPHG | 803 | TSPSGRSANIIG |
| 771 | TSTSGRSANPIG | 804 | TSPSGRSANFIG |
| 772 | TSTSGRSANPLG | 805 | TSPTGRSANPIG |
| 773 | ISTSGRSANPSG | 806 | TSPSGRSAIPIG |
| 774 | TSTSGRSANPIG | 807 | TSYTGRSANPIG |
| 775 | YSTSGRSANPIG | 808 | TSYSGRSAIPIG |
| 776 | TSYSGRSAVPAG | 809 | TSISGRSANYIG |
| 777 | TSPSGRSANIAG | 810 | TSPSGRSAGPIG |
| 778 | TSPSGRSANFAG | 811 | TSYTGRSAVPIG |
| 779 | TSPTGRSANPAG | 812 | TSYTGRSAVYIG |
| 780 | TSPSGRSAIPAG | 813 | TSYTGRSAVVIG |
| 781 | TSYTGRSANPAG | 814 | TSYTGRSAVHIG |
| 782 | TSYSGRSAIPAG | 815 | TSYSGRSAVPLG |
| 783 | TSISGRSANYAG | 816 | TSPSGRSANILG |
| 784 | TSPSGRSAGPAG | 817 | TSPSGRSANFLG |
| 818 | TSPTGRSANPLG | 851 | ISYTGRSAVYIG |
| 819 | TSPSGRSAIPLG | 852 | ISYTGRSAVVIG |
| 820 | TSYTGRSANPLG | 853 | ISYTGRSAVHIG |
| 821 | TSYSGRSAIPLG | 854 | YSYSGRSAVPIG |
| 822 | TSISGRSANYLG | 855 | YSPSGRSANTIG |
| 823 | TSPSGRSAGPLG | 856 | YSPSGRSANPIG |
| 824 | TSYTGRSAVPLG | 857 | YSPTGRSANPIG |
| 825 | TSYTGRSAVYLG | 858 | YSPSGRSAIPIG |
| 826 | TSYTGRSAVVLG | 859 | YSYTGRSANPIG |
| 827 | TSYTGRSAVHLG | 860 | YSYSGRSAIPIG |
| 828 | TSYSGRSAVPSG | 861 | YSISGRSANYIG |
| 829 | TSPSGRSANISG | 862 | YSPSGRSAGPIG |
| 830 | TSPSGRSANFSG | 863 | YSYTGRSAVPIG |
| 831 | TSPTGRSANPSG | 864 | YSYTGRSAVYIG |
| 832 | TSPSGRSAIPSG | 865 | YSYTGRSAVVIG |
| 833 | TSYTGRSANPSG | 866 | YSYTGRSAVHIG |
| 834 | TSYSGRSAIPSG | 867 | TSYTORSAVPRG |
| 835 | TSISGRSANYSG | 868 | TSYSGRSAVVRG |
| 836 | TSPSGRSAGPSG | 869 | TSYTGRSAVYRG |
| 837 | TSYTGRSAVPSG | 870 | TSYTGRSAVHRG |
| 838 | TSYTGRSAVYSG | 873 | TSTSGRSANPRG |
| 839 | TSYTGRSAVVSG | 877 | GGSGGSGGSTSTSGRSANPRGGGSGGSGGS |
| 840 | TSYTGRSAVHSG | 879 | GGSTSTSGRSANPRGGGS |
| 841 | ISYSGRSAVPIG | 967 | TSGSGRSATPRG |
| 842 | ISPSGRSANIIG | 969 | GGGGSGGGGSGGGGSGGGGS |
| 843 | ISPSGRSANFIG | 991 | GGGGSTSTSGRSANPRGGGGGS |
| 844 | ISPTGRSANPIG | | |
| 845 | ISPSGRSAIPIG | | |
| 846 | ISYTGRSANPIG | | |
| 847 | ISYSGRSAIPIG | | |
| 848 | ISISGRSANYIG | | |
| 849 | ISPSGRSAGPIG | | |
| 850 | ISYTGRSAVPIG | | |

The following sequence may also be used as a protease cleavage sequence:

(SEQ ID NO: 96)
X1-X2-X3-X4-X5-X6-X7-X8

wherein, X1 to X8 each represent a single amino acid, X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; and X8 is an amino acid selected from A, D, E, F, G, H, I, K, L, M, N, P, Q, R, S, T, V, W and Y.

The following sequence may also be used as a protease cleavage sequence:

(SEQ ID NO: 97)
X1-X2-X3-X4-X5-X6-X7-X8

wherein, X1 to X8 each represent a single amino acid, X1 is an amino acid selected from A, E, F, G, H, K, M, N, P, Q, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y.

The following sequence may also be used as a protease cleavage sequence:

(SEQ ID NO: 98)
X1-X2-X3-X4-X5-X6-X7-X8

wherein, X1 to X8 each represent a single amino acid, X1 is an amino acid selected from A, D, E, F, G, H, I, K, M, N, P, Q, S, T, W and Y; X2 is an amino acid selected from A, D, F, L, M, P, Q, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y.

The following sequence may also be used as a protease cleavage sequence:

(SEQ ID NO: 99)
X1-X2-X3-X4-X5-X6-X7-X8

wherein, X1 to X8 each represent a single amino acid, X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, E, F, H, I, K, L, M, N, P, Q, R, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y.

The following sequence may also be used as a protease cleavage sequence:

(SEQ ID NO: 100)
X1-X2-X3-X4-X5-X6-X7-X8

wherein, X1 to X8 each represent a single amino acid, X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, G, H, I, K, L, M, N, Q, R, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y.

The following sequence may also be used as a protease cleavage sequence:

(SEQ ID NO: 101)
X1-X2-X3-X4-X5-X6-X7-X8

wherein, X1 to X8 each represent a single amino acid, X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from E, F, K, M, N, P, Q, R, S and W; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y.

The following sequence may also be used as a protease cleavage sequence:

(SEQ ID NO: 102)
X1-X2-X3-X4-X5-X6-X7-X8

wherein, X1 to X8 each represent a single amino acid, X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, F, G, L, M, P, Q, V and W; X8 is an amino acid selected from A, D, E, F, G, H, I, K, L, M, N, P, Q, R, S, T, V, W and Y.

The following sequence may also be used as a protease cleavage sequence:

(SEQ ID NO: 103)
X1-X2-X3-X4-X5-X6-X7-X8

wherein, X1 to X8 each represent a single amino acid, X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, I, K, N, T and W

The following sequence may also be used as a protease cleavage sequence: X1-X2-X3-X4-X5-X6-X7-X8 (SEQ ID NO: 104)

(SEQ ID NO: 104)
X1-X2-X3-X4-X5-X6-X7-X8

wherein, X1 to X8 each represent a single amino acid, X1 is an amino acid selected from A, G, I, P, Q, S and Y; X2 is an amino acid selected from K or T; X3 is G; X4 is R; X5 is S; X6 is A; X7 is an amino acid selected from H, I and V; X8 is an amino acid selected from H, V and Y.

The following sequence may also be used as a protease cleavage sequence:

(SEQ ID NO: 105)
X1-X2-X3-X4-X5-X6-X7-X8

wherein, X1 to X8 each represent a single amino acid, X1 is Y; X2 is an amino acid selected from S and T; X3 is G; X4 is R; X5 is S; X6 is an amino acid selected from A and E; X7 is an amino acid selected from N and V; X8 is an amino acid selected from H, P, V and Y.

The following sequence may also be used as a protease cleavage sequence:

(SEQ ID NO: 106)
X1-X2-X3-X4-X5-X6-X7-X8-X9

wherein, X1 to X9 each represent a single amino acid, X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y; X9 is an amino acid selected from A, G, H, I, L and R.

The following sequence may also be used as a protease cleavage sequence:

(SEQ ID NO: 107)
X1-X2-X3-X4-X5-X6-X7-X8-X9

wherein, X1 to X9 each represent a single amino acid, X1 is an amino acid selected from A, E, F, G, H, K, M, N, P, Q, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y; X9 is an amino acid selected from A, G, H, I, L and R.

The following sequence may also be used as a protease cleavage sequence:

(SEQ ID NO: 108)
X1-X2-X3-X4-X5-X6-X7-X8-X9

wherein, X1 to X9 each represent a single amino acid, X1 is an amino acid selected from A, D, E, F, G, H, I, K, M, N, P, Q, S, T, W and Y; X2 is an amino acid selected from A, D, F, L, M, P, Q, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y; X9 is an amino acid selected from A, G, H, I, L and R.

The following sequence may also be used as a protease cleavage sequence:

| (SEQ ID NO: 109) | |
|---|---|
| | X1-X2-X3-X4-X5-X6-X7-X8-X9 |

wherein, X1 to X9 each represent a single amino acid, X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, E, F, H, I, K, L, M, N, P, Q, R, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y; X9 is an amino acid selected from A, G, H, I, L and R.

The following sequence may also be used as a protease cleavage sequence:

| (SEQ ID NO: 110) | |
|---|---|
| | X1-X2-X3-X4-X5-X6-X7-X8-X9 |

wherein, X1 to X9 each represent a single amino acid, X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, G, H, I, K, L, M, N, Q, R, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y; X9 is an amino acid selected from A, G, H, I, L and R.

The following sequence may also be used as a protease cleavage sequence:

| (SEQ ID NO: 111) | |
|---|---|
| | X1-X2-X3-X4-X5-X6-X7-X8-X9 |

wherein, X1 to X9 each represent a single amino acid, X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from E, F, K, M, N, P, Q, R, S and W; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y; X9 is an amino acid selected from A, G, H, I, L and R.

The following sequence may also be used as a protease cleavage sequence:

| (SEQ ID NO: 112) | |
|---|---|
| | X1-X2-X3-X4-X5-X6-X7-X8-X9 |

wherein, X1 to X9 each represent a single amino acid, X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, F, G, L, M, P, Q, V and W; X8 is an amino acid selected from A, D, E, F, G, H, I, K, L, M, N, P, Q, R, S, T, V, W and Y; X9 is an amino acid selected from A, G, H, I, L and R.

The following sequence may also be used as a protease cleavage sequence:

| (SEQ ID NO: 113) | |
|---|---|
| | X1-X2-X3-X4-X5-X6-X7-X8-X9 |

wherein, X1 to X9 each represent a single amino acid, X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, I, K, N, T and W; X9 is an amino acid selected from A, G, H, I, L and R.

The following sequence may also be used as a protease cleavage sequence:

| (SEQ ID NO: 114) | |
|---|---|
| | X1-X2-X3-X4-X5-X6-X7-X8-X9 |

wherein, X1 to X9 each represent a single amino acid, X1 is an amino acid selected from A, G, I, P, Q, S and Y; X2 is an amino acid selected from K or T; X3 is G; X4 is R; X5 is S; X6 is A; X7 is an amino acid selected from H, I and V; X8 is an amino acid selected from H, V and Y; X9 is an amino acid selected from A, G, H, I, L and R.

The following sequence may also be used as a protease cleavage sequence:

| (SEQ ID NO: 115) | |
|---|---|
| | X1-X2-X3-X4-X5-X6-X7-X8-X9 |

wherein, X1 to X9 each represent a single amino acid, X1 is Y; X2 is an amino acid selected from S and T; X3 is G; X4 is R; X5 is S; X6 is an amino acid selected from A and E; X7 is an amino acid selected from N and V; X8 is an amino acid selected from H, P, V and Y; X9 is an amino acid selected from A, G, H, I, L and R.

The following sequence may also be used as a protease cleavage sequence:

| (SEQ ID NO: 116) | |
|---|---|
| | X10-X11-X1-X2-X3-X4-X5-X6-X7-X8 |

wherein, X1 to X11 each represent a single amino acid, X10 is an amino acid selected from I, T and Y; X11 is S; X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y.

The following sequence may also be used as a protease cleavage sequence:

| (SEQ ID NO: 117) | |
|---|---|
| | X10-X11-X1-X2-X3-X4-X5-X6-X7-X8 |

wherein, X1 to X1I each represent a single amino acid, X10 is an amino acid selected from I, T and Y; X11 is S; X1 is an amino acid selected from A, E, F, G, H, K, M, N, P, Q, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y.

The following sequence may also be used as a protease cleavage sequence:

| (SEQ ID NO: 118) | |
|---|---|
| | X10-X11-X1-X2-X3-X4-X5-X6-X7-X8 |

wherein, X1 to X11 each represent a single amino acid, X10 is an amino acid selected from I, T and Y; X11 is S; X1 is an amino acid selected from A, D, E, F, G, H, I, K, M, N, P, Q, S, T, W and Y; X2 is an amino acid selected from A, D, F, L, M, P, Q, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y.

The following sequence may also be used as a protease cleavage sequence:

| (SEQ ID NO: 119) | |
|---|---|
| | X10-X11-X1-X2-X3-X4-X5-X6-X7-X8 |

wherein, X1 to X11 each represent a single amino acid, X10 is an amino acid selected from I, T and Y; X11 is S; X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, E, F, H, I, K, L, M, N, P, Q, R, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y.

The following sequence may also be used as a protease cleavage sequence:

| (SEQ ID NO: 120) | |
|---|---|
| | X10-X11-X1-X2-X3-X4-X5-X6-X7-X8 |

wherein, X1 to X11 each represent a single amino acid, X10 is an amino acid selected from I, T and Y; X11 is S; X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, G, H, I, K, L, M, N, Q, R, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y.

The following sequence may also be used as a protease cleavage sequence:

| (SEQ ID NO: 121) | |
|---|---|
| | X10-X11-X1-X2-X3-X4-X5-X6-X7-X8 |

wherein, X1 to X11 each represent a single amino acid, X10 is an amino acid selected from I, T and Y; X11 is S; X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from E, F, K, M, N, P, Q, R, S and W; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y.

The following sequence may also be used as a protease cleavage sequence:

| (SEQ ID NO: 122) | |
|---|---|
| | X10-X11-X1-X2-X3-X4-X5-X6-X7-X8 |

wherein, X1 to X11 each represent a single amino acid, X10 is an amino acid selected from I, T and Y; X11 is S; X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, F, G, L, M, P, Q, V and W; X8 is an amino acid selected from A, D, E, F, G, H, I, K, L, M, N, P, Q, R, S, T, V, W and Y.

The following sequence may also be used as a protease cleavage sequence:

| (SEQ ID NO: 123) | |
|---|---|
| | X10-X11-X1-X2-X3-X4-X5-X6-X7-X8 |

wherein, X1 to X11 each represent a single amino acid, X10 is an amino acid selected from I, T and Y; X11 is S; X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, I, K, N, T and W.

The following sequence may also be used as a protease cleavage sequence:

(SEQ ID NO: 124)
X10-X11-X1-X2-X3-X4-X5-X6-X7-X8

wherein, X1 to X11 each represent a single amino acid, X10 is an amino acid selected from I, T and Y; X11 is S; X1 is an amino acid selected from A, G, I, P, Q, S and Y; X2 is an amino acid selected from K or T; X3 is G; X4 is R; X5 is S; X6 is A; X7 is an amino acid selected from H, I and V; X8 is an amino acid selected from H, V and Y.

The following sequence may also be used as a protease cleavage sequence:

| (SEQ ID NO: 125) | |
|---|---|
| | X10-X11-X1-X2-X3-X4-X5-X6-X7-X8 |

wherein, X1 to X11 each represent a single amino acid, X10 is an amino acid selected from I, T and Y; X11 is S; X1 is Y; X2 is an amino acid selected from S and T; X3 is G; X4 is R; X5 is S; X6 is an amino acid selected from A and E; X7 is an amino acid selected from N and V; X8 is an amino acid selected from H, P, V and Y.

The following sequence may also be used as a protease cleavage sequence:

| (SEQ ID NO: 126) | |
|---|---|
| | X10-X11-X1-X2-X3-X4-X5-X6-X7-X8-X9 |

wherein, X1 to X11 each represent a single amino acid, X10 is an amino acid selected from I, T and Y; X11 is S; X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y; X9 is an amino acid selected from A, G, H, I, L and R.

The following sequence may also be used as a protease cleavage sequence: X10-X11-X1-X2-X3-X4-X5-X6-X7-X8-X9 (SEQ ID NO: 127)

| (SEQ ID NO: 127) | |
|---|---|
| | X10-X11-X1-X2-X3-X4-X5-X6-X7-X8-X9 |

wherein, X1 to X11 each represent a single amino acid, X10 is an amino acid selected from I, T and Y; X11 is S; X1 is an amino acid selected from A, E, F, G, H, K, M, N, P, Q, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y; X9 is an amino acid selected from A, G, H, I, L and R.

The following sequence may also be used as a protease cleavage sequence:

| (SEQ ID NO: 128) | |
|---|---|
| | X10-X11-X1-X2-X3-X4-X5-X6-X7-X8-X9 |

wherein, X1 to X11 each represent a single amino acid, X10 is an amino acid selected from I, T and Y; X11 is S; X1 is an amino acid selected from A, D, E, F, G, H, I, K, M, N, P, Q, S, T, W and Y; X2 is an amino acid selected from A, D, F, L, M, P, Q, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y; X9 is an amino acid selected from A, G, H, I, L and R.

The following sequence may also be used as a protease cleavage sequence:

| (SEQ ID NO: 129) | |
|---|---|
| | X10-X11-X1-X2-X3-X4-X5-X6-X7-X8-X9 |

wherein, X1 to X11 each represent a single amino acid, X10 is an amino acid selected from I, T and Y; X11 is S; X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, E, F, H, I, K, L, M, N, P, Q, R, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y; X9 is an amino acid selected from A, G, H, I, L and R.

The following sequence may also be used as a protease cleavage sequence:

| (SEQ ID NO: 130) | |
|---|---|
| | X10-X11-X1-X2-X3-X4-X5-X6-X7-X8-X9 |

wherein, X1 to X11 each represent a single amino acid, X10 is an amino acid selected from I, T and Y; X11 is S; X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, G, H, I, K, L, M, N, Q, R, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y; X9 is an amino acid selected from A, G, H, I, L and R.

The following sequence may also be used as a protease cleavage sequence:

| (SEQ ID NO: 131) | |
|---|---|
| | X10-X11-X1-X2-X3-X4-X5-X6-X7-X8-X9 |

wherein, X1 to X11 each represent a single amino acid, X10 is an amino acid selected from I, T and Y; X11 is S; X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from E, F, K, M, N, P, Q, R, S and W; X7 is an amino acid selected from A, D, E, F, G, H, I, K, L, M, N, P, Q, R, S, T, V, W and Y; X8 is an amino acid selected from A, D, E, F, G, H, I, K, L, M, N, P, Q, R, S, T, V, W and Y; X9 is an amino acid selected from A, G, H, I, L and R.

The following sequence may also be used as a protease cleavage sequence:

| (SEQ ID NO: 132) | |
|---|---|
| | X10-X11-X1-X2-X3-X4-X5-X6-X7-X8-X9 |

wherein, X1 to X11 each represent a single amino acid, X10 is an amino acid selected from I, T and Y; X11 is S; X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, F, G, L, M, P, Q, V and W; X8 is an amino acid selected from A, D, E, F, G, H, I, K, L, M, N, P, Q, R, S, T, V, W and Y; X9 is an amino acid selected from A, G, H, I, L and R.

The following sequence may also be used as a protease cleavage sequence:

| (SEQ ID NO: 133) | |
|---|---|
| | X10-X11-X1-X2-X3-X4-X5-X6-X7-X8-X9 |

wherein, X1 to X11 each represent a single amino acid, X10 is an amino acid selected from I, T and Y; X11 is S; X1 is an amino acid selected from A, D, E, F, G, H, I, K, M, N, P, Q, S, T, W and Y; X2 is an amino acid selected from A, D, E, F, H, K, L, M, P, Q, S, T, V, W and Y; X3 is an amino acid selected from A, D, E, F, G, H, I, K, L, M, N, P, Q, R, S, T, V, W and Y; X4 is R; X5 is an amino acid selected from A, D, E, F, G, H, I, K, L, M, N, P, Q, R, S, T, V, W and Y; X6 is an amino acid selected from A, D, E, F, H, I, K, L, M, N, P, Q, R, S, T, V, W and Y; X7 is an amino acid selected from A, D, E, F, G, H, I, K, L, M, N, P, Q, R, S, T, V, W and Y; X8 is an amino acid selected from A, D, E, F, G, I, K, N, T and W; X9 is an amino acid selected from A, G, H, I, L and R.

The following sequence may also be used as a protease cleavage sequence:

| (SEQ ID NO: 134) | |
|---|---|
| | X10-X11-X1-X2-X3-X4-X5-X6-X7-X8-X9 |

wherein, X1 to X11 each represent a single amino acid, X10 is an amino acid selected from I, T and Y; X11 is S; X1 is an amino acid selected from A, G, I, P, Q, S and Y; X2 is an amino acid selected from K or T; X3 is G; X4 is R; X5 is S; X6 is A; X7 is an amino acid selected from H, I and V; X8 is an amino acid selected from H, V and Y; X9 is an amino acid selected from A, G, H, I, L and R.

The following sequence may also be used as a protease cleavage sequence:

| (SEQ ID NO: 135) | |
|---|---|
| | X10-X11-X1-X2-X3-X4-X5-X6-X7-X8-X9 |

wherein, X1 to X11 each represent a single amino acid, X10 is an amino acid selected from I, T and Y; X11 is S; X1 is Y; X2 is an amino acid selected from S and T; X3 is G; X4 is R; X5 is S; X6 is an amino acid selected from A and E; X7 is an amino acid selected from N and V; X8 is an amino acid selected from H, P, V and Y; X9 is an amino acid selected from A, G, H, I, L and R.

In addition to using the above-mentioned protease cleavage sequences, novel protease cleavage sequences may also be obtained by screening. For example, based on the result of crystal structure analysis of a known protease cleavage sequence, novel protease cleavage sequences can be explored by changing the interaction of active residues/recognition residues of the cleavage sequence and the enzyme. Novel protease cleavage sequences can also be explored by altering amino acids in a known protease cleavage sequence and examining interaction between the altered sequence and the protease. As another example, protease cleavage sequences can be explored by examining interaction of the protease with a library of peptides displayed using an in vitro display method such as phage display and ribosome display, or with an array of peptides immobilized on a chip or beads. Interaction between a protease cleavage sequence and a protease can be examined by testing cleavage of the sequence by the protease in vitro or in vivo.

Cleavage fragments after protease treatment can be separated by electrophoresis such as SDS-PAGE and quantified to evaluate the protease cleavage sequence, the activity of the protease, and the cleavage ratio of a molecule into which the protease cleavage sequence has been introduced. A non-limiting embodiment of the method of evaluating the cleavage ratio of a molecule into which a protease cleavage sequence has been introduced includes the following method: For example, when the cleavage ratio of an antibody variant into which a protease cleavage sequence has been introduced is evaluated using recombinant human u-Plasminogen Activator/Urokinase (human uPA, huPA) (R&D Systems; 1310-SE-010) or recombinant human Matriptase/ST14 Catalytic Domain (human MT-SP1, hMT-SP1) (R&D Systems; 3946-SE-010), 100 microgram/mL of the antibody variant is reacted with 40 nM huPA or 3 nM hMT-SP1 in PBS at 37 degrees C. for one hour, and then subjected to capillary electrophoresis immunoassay. Capillary electrophoresis immunoassay can be performed using Wes (Protein Simple), but the present method is not limited thereto. As an alternative to capillary electrophoresis immunoassay, SDS-PAGE and such may be performed for separation, followed by detection with Western blotting. The present method is not limited to these methods. Before and after cleavage, the light chain can be detected using anti-human lambda chain HRP-labelled antibody (abeam; ab9007), but any antibody that can detect cleavage fragments may be used. The area of each peak obtained after protease treatment is output using software for Wes (Compass for SW; Protein Simple), and the cleavage ratio (%) of the antibody variant can be determined with the following formula: (Peak area of cleaved light chain)×100/(Peak area of cleaved light chain+Peak area of uncleaved light chain). Cleavage ratios can be determined if protein fragments are detectable before and after protease treatment. Cleavage ratios can be determined not only for antibody variants but also for various protein molecules into which a protease cleavage sequence has been introduced.

The in vivo cleavage ratio of a molecule into which a protease cleavage sequence has been introduced can be determined by administering the molecule into animals and detecting the administered molecule in blood samples. For example, an antibody variant into which a protease cleavage sequence has been introduced is administered to mice, and plasma is collected from their blood samples. The antibody is purified from the plasma by a method known to those skilled in the art using Dynabeads Protein A (Thermo; 10001D), and then subjected to capillary electrophoresis immunoassay to evaluate the protease cleavage ratio of the antibody variant. Capillary electrophoresis immunoassay can be performed using Wes (Protein Simple), but the present method is not limited thereto. As an alternative to capillary electrophoresis immunoassay, SDS-PAGE and such may be performed for separation, followed by detection with Western blotting. The present method is not limited to these methods. The light chain of the antibody variant collected from mice can be detected using anti-human lambda chain HRP-labelled antibody (abeam; ab9007), but any antibody that can detect cleavage fragments may be used. Once the area of each peak obtained by capillary electrophoresis immunoassay is output using software for Wes (Compass for SW; Protein Simple), the ratio of the remaining light chain can be calculated as [Peak area of light chain]/[Peak area of heavy chain] to determine the ratio of the full-length light chain that remain uncleaved in the mouse body. In vivo cleavage efficiencies can be determined if protein fragments collected from a living organism are detectable. Cleavage ratios can be determined not only for antibody variants but also for various protein molecules into which a protease cleavage sequence has been introduced. Calculation of cleavage ratios by the above-mentioned methods enables, for example, comparison of the in vivo cleavage ratios of antibody variants into which different cleavage sequences have been introduced, and comparison of the cleavage ratio of a single antibody variant between different animal models such as a normal mouse model and a tumour-grafted mouse model.

For example, the protease cleavage sequences shown in Table 1 have all been disclosed in WO2019/107384. Polypeptides containing these protease cleavage sequences are all useful as protease substrates which are hydrolysed by the action of proteases. Thus, the present invention provides protease substrates comprising a sequence selected from those described herein such as SEQ ID NOs: 96-135 above, and the sequences listed in Table 1. The protease substrates of the present invention can be utilized as, for example, a library from which one with properties that suit the purpose can be selected to incorporate into a ligand-binding moiety or molecule. Specifically, in order to cleave the ligand-binding moiety/molecule selectively by a protease localized in the lesion, the substrates can be evaluated for sensitivity to that protease. When a ligand-binding moiety/molecule connected with a ligand moiety/molecule is administered in vivo, the molecule may come in contact with various proteases before reaching the lesion. Therefore, the molecule should preferably have sensitivity to the protease localized to the lesion and also as high resistance as possible to the other proteases. In order to select a desired protease cleavage sequence depending on the purpose, each protease substrate can be analysed in advance for sensitivity to various proteases exhaustively to find its protease resistance. Based on the obtained protease resistance spectra, it is possible to find a protease cleavage sequence with necessary sensitivity and resistance. Alternatively, a ligand-binding molecule into which a protease cleavage sequence has been incorporated undergoes not only enzymatic actions by proteases but also various environmental stresses such as pH changes, temperature, and oxidative/reductive stress, before reaching the lesion. Resistance to these external factors can also be compared among the protease substrates, and this comparative information can be used to select a protease cleavage sequence with desired properties depending on the purpose.

### Peptide Linker(s)

In one embodiment of the present invention, a flexible linker is further attached to one end or both ends of each protease cleavage site. The flexible linker attached to one end of the first protease cleavage site is referred to as “first flexible linker”, and the flexible linker attached to the other end as “second flexible linker”. When the fusion protein of the present invention contains two or more protease cleavage sites, similarly, the flexible linkers attached to the second protease cleavage site are referred to as “third flexible linker” and “fourth flexible linker, and the flexible linkers attached to the third protease cleavage site are referred to as “fifth flexible linker” and “sixth flexible linker, and so on. The descriptions below are provided to explain the first and second flexible linkers attached to the first protease cleavage site, but also similarly apply to the third and subsequent flexible linkers attached to the second and subsequent protease cleavage sites.

In a particular embodiment, the protease cleavage site and the flexible linker have any of the following formulas:
(protease cleavage site),
(first flexible linker)-(protease cleavage site),
(protease cleavage site)-(second flexible linker), and
(first flexible linker)-(protease cleavage site)-(second flexible linker).

The flexible linker according to the present embodiment is preferably a peptide linker. The first flexible linker and the second flexible linker each independently and arbitrarily exist and are identical or different flexible linkers each containing at least one flexible amino acid (Gly, etc.). The flexible linker contains, for example, a sufficient number of residues (amino acids arbitrarily selected from Arg, Ile, Gln, Glu, Cys, Tyr, Trp, Thr, Val, His, Phe, Pro, Met, Lys, Gly, Ser, Asp, Asn, Ala, etc., particularly Gly, Ser, Asp, Asn, and Ala, in particular, Gly and Ser, especially Gly, etc.) for the protease cleavage sequence to obtain the desired protease accessibility.

The flexible linker suitable for use at both ends of the protease cleavage sequence is usually a flexible linker that improves the access of protease to the protease cleavage sequence and elevates the cleavage efficiency of the protease. A suitable flexible linker may be readily selected and can be preferably selected from among different lengths such as 1 amino acid (Gly, etc.) to 20 amino acids, 2 amino acids to 15 amino acids, or 3 amino acids to 12 amino acids including 4 amino acids to 10 amino acids, 5 amino acids to 9 amino acids, 6 amino acids to 8 amino acids, or 7 amino acids to 8 amino acids. In some embodiments of the present invention, the flexible linker is a peptide linker of 1 to 7 amino acids.

Examples of the flexible linker include, but are not limited to, glycine polymers (G)n, glycine-serine polymers (including e.g., (GS)n, (GSGGS: SEQ ID NO: 145)n and (GGGS: SEQ ID NO: 136)n, wherein n is an integer of at least 1), glycine-alanine polymers, alanine-serine polymers, and other flexible linkers well known in conventional techniques.

Among them, glycine and glycine-serine polymers are receiving attention because these amino acids are relatively unstructured and easily function as neutral tethers between components.

Examples of the flexible linker consisting of the glycine-serine polymer can include, but are not limited to,

Ser
Gly Ser (GS)
Ser Gly (SG)
Gly Ser (GGS)
Gly Ser Gly (GSG)
Ser Gly Gly (SGG)
Gly Ser Ser (GSS)
Ser Ser Gly (SSG)
Ser Gly Ser (SGS)
Gly Gly Gly Ser (GGGS, SEQ ID NO: 136)
Gly Gly Ser Gly (GGSG, SEQ ID NO: 137)
Gly Ser Gly Gly (GSGG, SEQ ID NO: 138)
Ser Gly Gly Gly (SGGG, SEQ ID NO: 139)
Gly Ser Ser Gly (GSSG, SEQ ID NO: 140)
Gly Gly Gly Gly Ser (GGGGS, SEQ ID NO: 141)
Gly Gly Gly Ser Gly (GGGSG, SEQ ID NO: 142)
Gly Gly Ser Gly Gly (GGSGG, SEQ ID NO: 143)
Gly Ser Gly Gly Gly (GSGGG, SEQ ID NO: 144)
Gly Ser Gly Gly Ser (GSGGS, SEQ ID NO: 145)
Ser Gly Gly Gly Gly (SGGGG, SEQ ID NO: 146)
Gly Ser Ser Gly Gly (GSSGG, SEQ ID NO: 147)
Gly Ser Gly Ser Gly (GSGSG, SEQ ID NO: 148)
Ser Gly Gly Ser Gly (SGGSG, SEQ ID NO: 149)
Gly Ser Ser Ser Gly (GSSSG, SEQ ID NO: 150)
Gly Gly Gly Gly Gly Ser (GGGGGS, SEQ ID NO: 151)
Ser Gly Gly Gly Gly Gly (SGGGGG, SEQ ID NO: 152)
Gly Gly Gly Gly Gly Gly Ser (GGGGGGS, SEQ ID NO:
153)
Ser Gly Gly Gly Gly Gly Gly (SGGGGGG, SEQ ID NO:
154)
(Gly Gly Gly Gly Ser (GGGGS, SEQ ID NO: 141))n
(Ser Gly Gly Gly Gly (SGGGG, SEQ ID NO: 146))n
(Gly Gly Ser Gly Gly (GGSGG, SEQ ID NO: 143))n

wherein n is an integer of 1 or larger.

However, the length and sequence of the peptide linker can be appropriately selected by those skilled in the art according to the purpose.

In some embodiments of the present invention, the ligand-binding moiety or molecule comprises a ligand-binding domain comprising antibody VH and antibody VL. Examples of the ligand-binding moiety/molecule comprising VH and VL include, but are not limited to, Fv, scFv, Fab, Fab′, Fab′-SH, F(ab′)₂, and full-length antibodies.

In some embodiments of the present invention, the ligand-binding moiety or molecule contains a Fc region. In the case of using an IgG antibody Fc region, its type is not limited, and, for example, IgG1, IgG2, IgG3, or IgG4 Fc region may be used. For example, a Fc region containing one sequence selected from the amino acid sequences represented by SEQ ID NOs: 155, 156, 157, and 158, or a Fc region mutant prepared by adding an alteration to the Fc region may be used. In some embodiments of the present invention, the ligand binding moiety/molecule comprises an antibody constant region. For instance, the heavy chain constant region of human IgG1, human IgG2, human IgG3, and human IgG4 are shown in SEQ ID NOs: 155 to 158, respectively. For instance, the Fc region of human IgG1, human IgG2, human IgG3, and human IgG4 are shown as a partial sequence of SEQ ID NOs: 155 to 158.

In some more specific embodiments of the present invention, the ligand-binding moiety or molecule is an antibody. In the case of using an antibody as the ligand-binding moiety/molecule, the binding to the ligand is achieved by a variable region. In some further specific embodiments, the ligand-binding moiety/molecule is an IgG antibody. In the case of using an IgG antibody as the ligand-binding moiety/molecule, its type is not limited, and IgG1, IgG2, IgG3, IgG4, or the like can be used. In the case of using an IgG antibody as the ligand-binding moiety/molecule, the binding to the ligand is also achieved by a variable region. One or both of the two variable regions of the IgG antibody can achieve the binding to the ligand. In the above-mentioned embodiments, the fusion protein of the present invention preferably comprises one ligand moiety (monovalent) or two ligand moieties (bivalent) which are connected with the C-terminal region of the antibody moiety via one or two peptide linkers. In some embodiments where the antibody is a bispecific antibody in which only one of the two variable regions binds to a ligand of interest, the fusion protein preferably comprises only one ligand moiety.

In some embodiments of the present invention, a domain having ligand binding activity is separated from the ligand-binding moiety/molecule by the cleavage of the protease cleavage site or the protease cleavage sequence in the ligand-binding moiety/molecule so that the binding to the ligand is attenuated or abolished. In an embodiment using an IgG antibody as the ligand-binding moiety/molecule, for example, one of the variable regions of the antibody is provided with a protease cleavage site or a protease cleavage sequence so that the antibody cannot form the full-length antibody variable region in a cleaved state, and thereby the binding to the ligand is attenuated or abolished.

In some embodiments of the present invention, the fusion protein is designed such that the protease cleavage site or protease cleavage sequence is provided in the ligand-binding moiety or molecule comprising a ligand-binding domain comprising antibody VH and antibody VL, and whereas the two peptides in the Fab structure have entire heavy chain-light chain interaction with each other before cleavage, the cleavage of the protease cleavage site or protease cleavage sequence results in attenuation or abolishment of the interaction between the peptide containing the VH (or a portion of the VH) and the peptide containing the VL (or a portion of the VL), reducing or abolishing the association between the VH and the VL.

### VH and VL Domains

In the present specification, the term “association” or “interaction” as used herein can refer to, for example, a state where two or more polypeptide regions associate or interact with each other. In general, a hydrophobic bond, a hydrogen bond, an ionic bond, or the like is formed between the intended polypeptide regions to form an associate. As one example of common association, an antibody typified by a natural antibody is known to retain a paired structure of a heavy chain variable region (VH) and a light chain variable region (VL) through a noncovalent bond or the like therebetween.

In some embodiments of the present invention, VH and VL contained in the ligand-binding domain associate with each other. The association between the antibody VH and the antibody VL may be abolished, for example, by the cleavage at the cleavage site or the protease cleavage sequence. The abolishment of the association can be used interchangeably with, for example, the whole or partial abolishment of the state where two or more polypeptide regions interact with each other. For the abolishment of the association between the VH and the VL, the interaction between the VH and the VL may be wholly abolished, or the interaction between the VH and the VL may be partially abolished. In some embodiments, the ligand-binding domain encompasses a ligand-binding moiety or molecule in which the association between antibody VL or a portion thereof and antibody VH or a portion thereof is abolished by the cleavage at the protease cleavage site or abolished by the cleavage of the protease cleavage sequence.

In some embodiments, the molecular weight of the fusion protein after protease cleavage at the protease cleavage site (“in the second state”) is smaller than the molecular weight of the fusion protein before protease cleavage at the protease cleavage site (“in the first state”). In some embodiments, the molecular weight of the VH, VL, or a portion of ligand-binding domain released upon cleavage at the protease cleavage sequence is approximately 26 kDa, or 13 kDa, or smaller. In some embodiments, the VH, or VL is released upon cleavage at the protease cleavage site, and the molecular weight of the released VH, or VL, is approximately 26 kDa, i.e. full-length VH and VL. In some embodiments, the ratio of the molecular weight of the fusion protein in the first state and the molecular weight of the fusion protein in the second state is 10:9, or the molecular weight of the fusion protein in the second state is 9/10 that of the molecular weight of the fusion protein in the first state, or the percentage reduction in molecular weight of the fusion protein in the second state compared to the fusion protein in the first state is 10%.

The term “dissociation” as used herein can refer to, the whole or partial abolishment of the abovementioned interactions. In some embodiments, the term “reduced association between VH and VL”, or “reduced interaction between VH and VL” may also be used interchangeably to refer to the whole or partial abolishment or attenuation of the association between the peptide containing the VH (or a portion of the VH) and the peptide containing the VL (or a portion of the VL). In further embodiments, reducing association or interaction is complete, leading to an abolishment of any association or interaction between VH and VL. In such cases, VH or VL completely dissociates from each other, also referred herein as “VH release”, or “VL release”. As used herein, “VH release” and “VL release” refers to the release of antibody VH, or fragment thereof, or fragment of the cleaved protein comprising VH, or fragment thereof, and the release of antibody VL, or fragment thereof, or fragment of the cleaved protein comprising VL, or fragment thereof, respectively.

In some embodiments of the present invention, the ligand-binding moiety/molecule comprises a ligand-binding domain comprising antibody VH and antibody VL, and the antibody VH and the antibody VL in the ligand-binding moiety/molecule are associated with each other in a state where the protease cleavage site or the protease cleavage sequence of the ligand-binding moiety/molecule is uncleaved, whereas the association between the antibody VH and the antibody VL in the ligand-binding moiety/molecule is abolished by the cleavage at the cleavage site or the protease cleavage sequence. The cleavage site or the protease cleavage sequence in the ligand-binding moiety/molecule may be placed at any position in the ligand-binding moiety/molecule as long as the ligand binding ability of the ligand-binding moiety/molecule can be attenuated or abolished by the cleavage of the cleavage site or the protease cleavage sequence.

In some embodiments, a ligand is fused to the antibody VH, and this fusion is released upon the protease cleavage (“VH-ligand release”). In some embodiments, a ligand is fused to the antibody VL, and this fusion is released upon the protease cleavage (“VL-ligand release”).

In some embodiments, the present invention also includes a bivalent homodimer fusion protein comprising a full-length IgG antibody comprising a protease cleavage site at the boundary between VH and CH1 or VL and CL of its variable region and a ligand binding to said variable region. Upon protease cleavage, either the VH or the VL dissociates from the fusion protein and the ligand dissociates from the variable region. The VH or VL dissociates from the fusion protein and the association between VL or a portion thereof and VH or a portion thereof is abolished by the cleavage at the protease cleavage site or abolished by the cleavage of the protease cleavage sequence.

In some embodiments, the present invention also includes a bivalent homodimer fusion protein comprising an IgG antibody-like polypeptide comprising a protease cleavage site at the boundary between VH and CH1 or VL and CL of its variable region and a ligand binding to said variable region. Upon protease cleavage, either the VH or the VL dissociates from the fusion protein and the ligand dissociates from the variable region. The VH or VL dissociates from the fusion protein and the association between VL or a portion thereof and VH or a portion thereof is abolished by the cleavage at the protease cleavage site or abolished by the cleavage of the protease cleavage sequence.

In a further aspect, the present invention also includes a polypeptide comprising at least one antigen-binding domain comprising a protease cleavage site, whereupon cleavage at the protease cleavage site, a domain adjacent to the protease cleavage site dissociates and the dissociation is promoted by at least one amino acid modification performed at the interface between said domain and a corresponding interacting domain. In some embodiments, the polypeptide is an antibody, such as an IgG1, IgG2, IgG3, IgG4, IgG-IgG, IgG-Fab, or CrossMab antibody. In further embodiments, the polypeptide may be monovalent or bivalent, and may be monospecific or bispecific. In some embodiments, the polypeptide is an antibody fragment, such as scFv, scFv-Fc, tandem scFv, Fab, tandem Fab, F(ab′)2, Fab2, Fab-scFv-Fc, F(ab′)2-scFv2, bispecific Fab2, trispecific Fab2, bispecific diabody, trispecific diabody, tandem diabody, triabody, tetrabody, minibody, bibody or tribody, or any other fragments comprising a VH domain associated with a VL domain. In one embodiment, the antibody fragment comprises an antigen-binding domain that comprises an antibody heavy chain variable region VH, or an antibody light chain variable region VL.

In one embodiment, a polypeptide comprises an antibody fragment having an antigen-binding domain comprising antibody heavy chain variable region VH and antibody light chain variable region VL associating with each other and a protease cleavage site located near the boundary of the antibody VH and antibody VL, e.g. in a scFv, scFv-Fc, minibody, diabody, triabody, or a protease cleavage site located near the boundary of the antibody VH and adjacent CH1 or near the boundary of the antibody VL and adjacent CL, e.g. in a Fab, F(ab′)2, Fab2. Upon protease cleavage at the protease cleavage site, the VH or VL dissociates from each other and the association between the antibody VH or a portion thereof and the antibody VL or a portion thereof is abolished by the cleavage of the protease cleavage sequence.

In one embodiment, a polypeptide comprises an antibody having an antigen-binding domain comprising antibody heavy chain variable region VH and antibody light chain variable region VL associating with each other and a protease cleavage site located near the boundary of the antibody VH and adjacent antibody CH1 or near the boundary of the antibody VL and adjacent antibody CL. Upon protease cleavage at the protease cleavage site, the VH or VL dissociates from the polypeptide and the association between antibody VL or a portion thereof and antibody VH or a portion thereof is abolished by the cleavage at the protease cleavage site or abolished by the cleavage of the protease cleavage sequence.

In one embodiment, a polypeptide comprises an IgG antibody, that is selected from the group consisting of IgG1, IgG2, IgG3, IgG4, IgG-IgG, IgG-Fab, or CrossMab antibody, having an antigen-binding domain comprising antibody heavy chain variable region VH and antibody light chain variable region VL associating with each other and a protease cleavage site located near the boundary of the antibody VH and adjacent antibody CH1 or near the boundary of the antibody VL and adjacent antibody CL. Upon protease cleavage at the protease cleavage site, the VH or VL dissociates from the polypeptide and the association between antibody VL or a portion thereof and antibody VH or a portion thereof is abolished by the cleavage at the protease cleavage site or abolished by the cleavage of the protease cleavage sequence.

In some embodiments of the present invention, the ligand-binding moiety or molecule comprises a ligand-binding domain comprising antibody VH, antibody VL, and an antibody constant region. In some embodiments of the present invention, the variable region of the moiety or molecule comprises an antigen-binding domain comprising antibody VH, antibody VL, and an antibody constant region. In some embodiments of the present invention, the antibody comprises an antigen-binding domain comprising antibody VH, antibody VL, and an antibody constant region. In other embodiments of the present invention, the antibody fragment comprises an antigen-binding domain comprising antibody VH and antibody VL. As mentioned by Rothlisberger et al. (J Mol Biol. 2005 Apr. 8; 347 (4): 773-89), it is known that the VH and VL domains or the CH and CL domains of an antibody interact with each other via many amino acid side chains. VH-CH1 and VL-CL are known to be capable of forming a stable structure as a Fab domain. As previously reported, amino acid side chains generally interact between VH and VL with a dissociation constant in the range of 10⁻⁵ M to 10⁻⁸ M. When only VH and VL domains exist, only a small proportion may form an associated state.

In one embodiment of the present invention, the protease cleavage site or the protease cleavage sequence is located within the antibody constant region. In a more specific embodiment, the protease cleavage site or the protease cleavage sequence is located on the variable region side with respect to amino acid position 140 (EU numbering) in an antibody heavy chain constant region, preferably on the variable region side with respect to amino acid position 122 (EU numbering) in an antibody heavy chain constant region. In some specific embodiments, the cleavage site or the protease cleavage sequence is introduced at any position in a sequence from antibody heavy chain constant region amino acid position 118 (EU numbering) to antibody heavy chain constant region amino acid position 140 (EU numbering). In another more specific embodiment, the cleavage site or the protease cleavage sequence is located on the variable region side with respect to amino acid position 130 (EU numbering) (Kabat numbering position 130) in an antibody light chain constant region, preferably on the variable region side with respect to amino acid position 113 (EU numbering) (Kabat numbering position 113) in an antibody light chain constant region or on the variable region side with respect to amino acid position 112 (EU numbering) (Kabat numbering position 112) in an antibody light chain constant region. In some specific embodiments, the cleavage site or the protease cleavage sequence is introduced at any position in a sequence from antibody light chain constant region amino acid position 108 (EU numbering) (Kabat numbering position 108) to antibody light chain constant region amino acid position 131 (EU numbering) (Kabat numbering position 131).

In one embodiment of the present invention, the protease cleavage site or the protease cleavage sequence is located within the antibody VH or within the antibody VL. In a more specific embodiment, the cleavage site or the protease cleavage sequence is located on the antibody constant region side with respect to amino acid position 7 (Kabat numbering) of the antibody VH, preferably on the antibody constant region side with respect to amino acid position 40 (Kabat numbering) of the antibody VH, more preferably on the antibody constant region side with respect to amino acid position 101 (Kabat numbering) of the antibody VH, further preferably on the antibody constant region side with respect to amino acid position 109 (Kabat numbering) of the antibody VH or on the antibody constant region side with respect to amino acid position 111 (Kabat numbering) of the antibody VH. In a more specific embodiment, the cleavage site or the protease cleavage sequence is located on the antibody constant region side with respect to amino acid position 7 (Kabat numbering) of the antibody VL, preferably on the antibody constant region side with respect to amino acid position 39 (Kabat numbering) of the antibody VL, more preferably on the antibody constant region side with respect to amino acid position 96 (Kabat numbering) of the antibody VL, further preferably on the antibody constant region side with respect to amino acid position 104 (Kabat numbering) of the antibody VL or on the antibody constant region side with respect to amino acid position 105 (Kabat numbering) of the antibody VL. In some more specific embodiments, the protease cleavage site or the protease cleavage sequence is introduced at a position of residues constituting a loop structure in the antibody VH or the antibody VL, and residues close to the loop structure. The loop structure in the antibody VH or the antibody VL refers to a moiety that does not form a secondary structure such as alpha-helix or beta-sheet, in the antibody VH or the antibody VL. Specifically, the position of the residues constituting the loop structure and the residues close to the loop structure can refer to the range of amino acid position 7 (Kabat numbering) to amino acid position 16 (Kabat numbering), amino acid position 40 (Kabat numbering) to amino acid position 47 (Kabat numbering), amino acid position 55 (Kabat numbering) to amino acid position 69 (Kabat numbering), amino acid position 73 (Kabat numbering) to amino acid position 79 (Kabat numbering), amino acid position 83 (Kabat numbering) to amino acid position 89 (Kabat numbering), amino acid position 95 (Kabat numbering) to amino acid position 99 (Kabat numbering), or amino acid position 101 (Kabat numbering) to amino acid position 113 (Kabat numbering) of the antibody VH, or amino acid position 7 (Kabat numbering) to amino acid position 19 (Kabat numbering), amino acid position 39 (Kabat numbering) to amino acid position 46 (Kabat numbering), amino acid position 49 (Kabat numbering) to amino acid position 62 (Kabat numbering), or amino acid position 96 (Kabat numbering) to amino acid position 107 (Kabat numbering) of the antibody VL.

In some more specific embodiments, the cleavage site or the protease cleavage sequence is introduced at any position in a sequence from amino acid position 7 (Kabat numbering) to amino acid position 16 (Kabat numbering), from amino acid position 40 (Kabat numbering) to amino acid position 47 (Kabat numbering), from amino acid position 55 (Kabat numbering) to amino acid position 69 (Kabat numbering), from amino acid position 73 (Kabat numbering) to amino acid position 79 (Kabat numbering), from amino acid position 83 (Kabat numbering) to amino acid position 89 (Kabat numbering), from amino acid position 95 (Kabat numbering) to amino acid position 99 (Kabat numbering), or from amino acid position 101 (Kabat numbering) to amino acid position 113 (Kabat numbering) of the antibody VH.

In some more specific embodiments, the cleavage site or the protease cleavage sequence is introduced at any position in a sequence from amino acid position 7 (Kabat numbering) to amino acid position 19 (Kabat numbering), from amino acid position 39 (Kabat numbering) to amino acid position 46 (Kabat numbering), from amino acid position 49 (Kabat numbering) to amino acid position 62 (Kabat numbering), or from amino acid position 96 (Kabat numbering) to amino acid position 107 (Kabat numbering) of the antibody VL.

In one embodiment of the present invention, the protease cleavage site or the protease cleavage sequence is located near the boundary between the antibody VH and the antibody constant region. The phrase “near the boundary between the antibody VH and the antibody heavy chain constant region” can refer to between amino acid position 101 (Kabat numbering) of the antibody VH and amino acid position 140 (EU numbering) of the antibody heavy chain constant region and can preferably refer to between amino acid position 109 (Kabat numbering) of the antibody VH and amino acid position 122 (EU numbering) of the antibody heavy chain constant region, or between amino acid position 111 (Kabat numbering) of the antibody VH and amino acid position 122 (EU numbering) of the antibody heavy chain constant region. When antibody VH is fused with an antibody light chain constant region, the phrase “near the boundary between the antibody VH and the antibody light chain constant region” can refer to between amino acid position 101 (Kabat numbering) of the antibody VH and amino acid position 130 (EU numbering) (Kabat numbering position 130) of the antibody light chain constant region and can preferably refer to between amino acid position 109 (Kabat numbering) of the antibody VH and amino acid position 113 (EU numbering) (Kabat numbering position 113) of the antibody light chain constant region, or between amino acid position 111 (Kabat numbering) of the antibody VH and amino acid position 112 (EU numbering) (Kabat numbering position 112) of the antibody light chain constant region.

In one embodiment, the cleavage site or the protease cleavage sequence is located near the boundary between the antibody VL and the antibody constant region. The phrase “near the boundary between the antibody VL and the antibody light chain constant region” can refer to between amino acid position 96 (Kabat numbering) of the antibody VL and amino acid position 130 (EU numbering) (Kabat numbering position 130) of the antibody light chain constant region, and can preferably refer to between amino acid position 104 (Kabat numbering) of the antibody VL and amino acid position 113 (EU numbering) (Kabat numbering position 113) of the antibody light chain constant region, or between amino acid position 105 (Kabat numbering) of the antibody VL and amino acid position 112 (EU numbering) (Kabat numbering position 112) of the antibody light chain constant region. When antibody VL is fused with an antibody heavy chain constant region, the phrase “near the boundary between the antibody VL and the antibody heavy chain constant region” can refer to between amino acid position 96 (Kabat numbering) of the antibody VL and amino acid position 140 (EU numbering) of the antibody heavy chain constant region, and can preferably refer to between amino acid position 104 (Kabat numbering) of the antibody VL and amino acid position 122 (EU numbering) of the antibody heavy chain constant region, or between amino acid position 105 (Kabat numbering) of the antibody VL and amino acid position 122 (EU numbering) of the antibody heavy chain constant region.

The ligand-binding moiety/molecule of the invention such as a fusion protein can be provided with a protease cleavage site or protease cleavage sequence at a plurality of positions selected from, for example, within the antibody constant region, within the antibody VH, within the antibody VL, near the boundary between the antibody VH and the antibody constant region, near the boundary between antibody VL and the antibody constant region, and near the boundary between the antibody VH and antibody VL. In other embodiments, the antibody variable region or antigen-binding domain of the ligand-binding moiety/molecule of the invention such as a fusion protein can be provided with a protease cleavage site or protease cleavage sequence at a plurality of positions selected from, for example, within the antibody constant region, within the antibody VH, within the antibody VL, near the boundary between the antibody VH and the antibody constant region, near the boundary between antibody VL and the antibody constant region, and near the boundary between antibody VH and antibody VL. In other embodiments, the antibody variable region or antigen-binding domain of the antibody can be provided with a protease cleavage site or protease cleavage sequence at a plurality of positions selected from, for example, within the antibody constant region, within the antibody VH, within the antibody VL, near the boundary between the antibody VH and the antibody constant region, near the boundary between antibody VL and the antibody constant region, and near the boundary between the antibody VH and antibody VL. In other embodiments, the antigen-binding domain of the antibody fragment can be provided with a protease cleavage site or protease cleavage sequence at a plurality of positions selected from, for example, within the antibody constant region, within the antibody VH, or within the antibody VL. Those skilled in the art with reference to the present invention can change the form of a molecule comprising antibody VH, antibody VL, and an antibody constant region, for example, by swapping the antibody VH with the antibody VL. Such a molecular form is also included in the scope of the present invention.

In some embodiments, the ligand-binding moiety/molecule of the invention such as a fusion protein comprises a ligand-binding domain further comprising at least one amino acid modification that reduces association between VH and VL in the cleaved state (“second state”) than in the uncleaved state (“first state”), particularly the modification of an amino acid present at the interface between the VH and the VL. In another embodiment of the present specification, the moiety/molecule such as a fusion protein comprises an IgG antibody having an antigen binding domain that binds to a ligand, further comprising at least one amino acid modification that reduces association between VH and VL in the cleaved state (“second state”) than in the uncleaved state (“first state”), particularly the modification of an amino acid present at the interface between the VH and the VL. In another embodiment of the present specification, a polypeptide or an antibody comprises an antigen binding domain further comprising at least one amino acid modification that reduces association between VH and VL in the cleaved state (“second state”) than in the uncleaved state (“first state”), particularly the modification of an amino acid present at the interface between the VH and the VL. In another embodiment of the present specification, an antibody fragment comprises an antigen binding domain further comprising at least one amino acid modification that reduces association between VH and VL in the cleaved state (“second state”) than in the uncleaved state (“first state”), particularly the modification of an amino acid present at the interface between the VH and the VL.

In each of the aforementioned embodiments, the dissociation of VH and VL from the fusion protein or polypeptide or the reduction in association between VH or a portion thereof and VL or a portion thereof comprised within said fusion protein or polypeptide is promoted by at least one amino acid modification performed at the interface between VH and VL that reduces association between VH and VL or reduces the interaction of VH with VL in the cleaved state (“second state”) than in the uncleaved state “(first state”). In respect of yet other abovementioned embodiments, the dissociation of VH and VL from the antibody or antibody fragment or the reduction in association between VH or a portion thereof and VL or a portion thereof comprised within said antibody or antibody fragment thereof is promoted by at least one amino acid modification performed at the interface between VH and VL that reduces association between VH and VL or reduces the interaction of VH with VL in the cleaved state (“second state”) than in the uncleaved state “(first state”). In some of the aforementioned embodiments, VH-ligand and VL-ligand is released from the fusion protein upon dissociation from the fusion protein when the amino acid modification is performed at the interface between VH and VL that reduces association between VH and VL or reduces the interaction of VH with VL in the cleaved state (“second state”) than in the uncleaved state (“first state”).

The term “interface” as used herein, refers to a face at which two regions (of amino acid residues) associate or interact with each other. Amino acid residues forming the interface are usually one or a plurality of amino acid residues contained in each polypeptide region subjected to association and dissociation and more preferably refer to amino acid residues that approach each other upon association or distance from each other during dissociation and participate in interaction. Specifically, the interaction includes noncovalent bonds such as a hydrogen bond, electrostatic interaction, or salt bridge formation between the amino acid residues approaching each other upon association or distancing from each other during dissociation.

The phrase “amino acid residues forming the interface” as used herein, specifically refers to amino acid residues contained in polypeptide regions constituting the interface. As one example, the polypeptide regions constituting the interface refer to polypeptide regions responsible for intramolecular or intermolecular selective binding in antibodies, ligands, receptors, substrates, etc. Specific examples of such polypeptide regions in antibodies can include heavy chain variable regions and light chain variable regions. In some embodiments of the present invention, examples of such polypeptide regions include the VH and VL regions, particularly, the framework regions (FR) within the VH and VL regions. Examples of the amino acid residues forming the interface include, but are not limited to, amino acid residues approaching each other upon association or distancing from each other during dissociation. Such amino acid residues can be identified, for example, by analysing the conformations of polypeptides and examining the amino acid sequences of polypeptide regions forming the interface upon association or dissociation of the polypeptides.

In some embodiments, an amino acid residue(s) forming the interface can be altered in order to promote the dissociation of or reduce the interaction or association between the heavy chain variable region and the light chain variable region. In a further specific embodiment, an amino acid residue(s) forming the interface between the VH on the heavy chain variable region and VL on the light chain variable region within the ligand-binding domain of a fusion protein or antigen-binding domain of an antibody or antibody fragment, can be altered. In a preferred embodiment, the amino acid residue(s) forming the interface can be altered by a method of introducing a mutation(s) to the interface amino acid residue(s) such that two or more amino acid residues forming the interface have the same charge(s). The alteration of the amino acid residue(s) to result in the same charge(s) includes the alteration of a positively charged amino acid residue(s) to a negatively charged amino acid residue(s) or an uncharged amino acid residue, the alteration of a negatively charged amino acid residue to a positively charged amino acid residue(s) or an uncharged amino acid residue(s), and the alteration of an uncharged amino acid residue(s) to a positively or negatively charged amino acid residue(s). Such an amino acid alteration is performed for the purpose of promoting the dissociation or reducing the interaction or association and is not limited by the position of the amino acid alteration or the type of the amino acid as long as the purpose of promoting the dissociation or reducing interaction can be achieved. Examples of the alteration include, but are not limited to, substitution.

In the present specification, the amino acid residue positions that may be altered to promote the dissociation of, or reduce the interaction or association between the VH and VL as described above, may be selected from one or more amino acids that are crucial in the association between VH and VL. The variable domains, VH and VL, comprises a conserved beta-barrel framework made up of two beta-sheets, one with four strands (A, B, D, E) and the other with six strands (A′, G, F, C, C″, C′″), with the strands GFC′C forming the interface between VH and VL (“VH/VL interface”). Studies of the VH/VL packing geometry reveals that 75% of the interface residues are constituted by framework beta-sheets and 25% by CDRs (inter-strand links between GF, BC and C′C″, respectively). Therefore, the majority of amino acid residues that are crucial for proper association of VH and VL into a VH/VL complex, i.e. Fv, resides within the framework beta-sheets. Multiple sequence alignment examining covariations between residues at all possible positions comprising more than 2000 V-class sequences of a variety of species such as human, mouse, cow, camel, llama, macaque and chicken, revealed that the majority of the most strongly conserved amino acids were positioned at the VH/VL interface. The highly conserved residues comprise for VL, amino acid positions Y36, Q37, P44, A43, L46, Y49, Y87, and F98, and for VH, amino acids V37, R38, G44, L45, E46, W47, Y91, H91 and W103. The identified positions occur within the VH/VL interface and predominantly alters the association between VH and VL. It has been demonstrated that mutations performed at one or more of the conserved amino acid positions affected the stability of the VH/VL complex and further affected the antigen binding capacity of the VH/VL complex (Sci Reports. (2017) 7, 12276). Since the amino acid positions critically involved in the association of VH and VL into Fv resides predominantly within the framework beta-sheets, and these same positions are highly conserved across the V regions of antibodies, it is expected that modifications at these selected amino acid positions will alter the affinity between VH and VL in the ligand-binding domain or antigen-binding domain of the presently described fusion proteins or IgG antibody-like polypeptides, regardless of the ligand or antigen identity. The skilled person looking to reduce the association between VH and VL would readily initate modifications in the identified amino acid positions and reasonably expect success to promote VH release, VH-ligand release, VL release or VL-ligand release. Without wishing to be limited thereto, by way of example, the following antibodies that bind to different target demonstrate highly conserved amino acids at the identified positions of Y36, Q37, P44, A43, L46, Y49, Y87, and F98 on the VL, and/or amino acid positions of V37, R38, G44, L45, E46, W47, Y91, H91 and W103 on the VH, said antibodies include rituximab, trastuzumab, alemtuzumab, cetuximab, bevacizumab, panitumumab, ofatumumab, ipilimumab, pertuzumab, obinutuzumab, ramucirumab, pembrolizumab, nivolumab, dinotuximab, daratumumab, necitumumab, elotuzumab, atezolizumab, olaratumab, avelumab, and duravlumab (Frontiers in Immunology. (2018) 8, 1751).

To reduce association between VH and VL and promote VH release, VH-ligand release, VL release or VL-ligand release from the fusion protein or IgG antibody-like polypeptide of the present specification, the skilled person would readily perform modifications at one or more of the aforementioned amino acid positions at the interface between VH and VL. In the present specification, the amino acid residues forming the interface between the VH and the VL include, but are not limited to, amino acid residues at positions 36, 37, 38, 44, 45, 46, 47, 49, 87, 91, 98, and 103 (J. Mol. Biol. (2005) 350, 112-125, Sci Reports. (2017) 7, 12276). Altering the amino acid residue(s) forming the interface between the VH and the VL, particularly, substitution of amino acid residue(s) can promote the dissociation of or reduce the interaction or association between the VH and VL. Examples of modifiable amino acid position(s) for substitution(s) include, but are not limited to 37, 38, 39, 44, 45, 46, 47, 91, and 103 on the VH, and, 36, 37, 38, 43, 44, 46, 49, 87 and 98 on the VL (according to Kabat numbering). Examples of such amino acid substitution(s) include, but are not limited to V37, R38, Q39, G44, L45, E46, W47, H91, Y91, and W103 on the VH, and, Y36, Q37, R38, A43, P44, L46, Y49, Y87 and F98 on the VL (according to Kabat numbering). Examples of such amino acid substitution(s) include, but are not limited to Q39D, W47A, W47L, W47M, Y91A, Y91L, Y91M, H91A, W103A, W103I, W103L, W103M, V37S, V37Q, G44Q, L45A, and L45Q on the VH, or R38E, Y49A, Y87A, Y87L, Y87M, F98A, F98L, F98M, A43Q, P44A, P44S, P44Q, L46E, and L46Q on the VL (according to Kabat numbering). For each modifiable amino acid position, the skilled person will alter said amino acid position to a resultant amino acid that achieves the purpose of reducing association between VH and VL, for example and not limiting thereto, when position W47 is selected, the skilled person may select either A, L or M. Alteration of amino acid residue(s) is not limited to VH or VL alone but also includes alteration on both VH and VL, as long as the purpose of promoting dissociation of VH or VL from the other can be achieved. Alteration of amino acid residue(s) can occur at any position(s) forming the interface between the VH and VL as long as it does not disrupt binding of the ligand to the ligand-binding domain of the fusion polypeptide or the binding of the antigen to an antigen-binding domain of a polypeptide, antibody or antibody fragment. Further, alteration of amino acid residue(s) does not have to occur in pairs as long as the alteration reduces association between the VH and VL without disrupting the binding activity of the ligand or antigen to the fusion protein or polypeptide. That is to say, alteration of amino acid residue(s) does not necessarily restrict the modifications in pairs, and includes any combination of amino acid modifications, for example, only one amino acid modification on the VH combined with two amino acid modifications on the VL or two amino acid modification on the VH combined with three amino acid modification on the VL. Alteration of amino acid residue can also be a single amino acid modification on the VH or the VL as long as the modification alone is capable of reducing the association between the VH and the VL but does not disrupt binding activity of the ligand or antigen to the fusion protein or polypeptide. For the avoidance of doubt, where amino acid modifications are performed on both the VH and the VL in at least a pair, the amino acid modification(s) performed on the VH and the amino acid modification(s) performed on the VL are not necessarily identical, and not necessarily different so long as the modifications are capable of reducing the association between the VH and the VL but does not disrupt binding activity of the ligand or antigen to the fusion protein or polypeptide. Confirmation that the amino acid modification(s) introduced at selected position(s) within the interface between VH and VL does not disrupt binding of the ligand to the ligand-binding domain of the fusion polypeptide or the binding of the antigen to an antigen-binding domain of a polypeptide or antibody may be conducted by any of the common methods described herein or as known to the skilled person.

In one embodiment, the amino acid modification are combinations of substitution of an amino acid present at the interface between the VH and the VL within the FR region. In a preferable embodiment, the combination substitutions are selected from the following groups (a) to (pp):
(a) L46Q and Y49A on VL;
(b) Q39D on the VH, and R38E on the VL;
(c) H91A on the VH, and L46Q and Y49A on the VL;
(d) Y91A on the VH, and A43Q and Y49A on the VL;
(e) Y91A on the VH, and P44A and Y49A on the VL;
(f) Y91A on the VH, and L46Q and Y49A on the VL;
(g) Y91A on the VH, and Y49A and Y87L on the VL;
(h) Y91M on the VH, and A43Q and Y49A on the VL;
(i) Y91M on the VH, and P44A and Y49A on the VL;
(j) Y91M on the VH, and L46Q and Y49A on the VL;
(k) Y91M on the VH, and Y49A and Y87L on the VL;
(l) Y91M on the VH, and Y49A and F98L on the VL;
(m) W103L on the VH, and A43Q and Y49A on the VL;
(n) W103L on the VH, and P44A and Y49A on the VL;
(o) W103L on the VH, and L46Q and Y49A on the VL;
(p) W103L on the VH, and Y49A and Y87L on the VL;
(q) W103I on the VH, and A43Q and Y49A on the VL;
(r) W103I on the VH, and P44A and Y49A on the VL;
(s) W103I on the VH, and L46Q and Y49A on the VL;
(t) W103M on the VH, and A43Q and Y49A on the VL;
(u) W103M on the VH, and P44A and Y49A on the VL;
(v) W103M on the VH, and L46Q and Y49A on the VL;
(w) W103M on the VH, and Y49A and Y87L on the VL;
(x) V37S on the VH, and A43Q and Y49A on the VL;
(y) V37S on the VH, and P44A and Y49A on the VL;
(z) V37S on the VH, and L46Q and Y49A on the VL;
(aa) V37S on the VH, and Y49A and Y87L on the VL;
(bb) V37S on the VH, and Y49A and F98L on the VL;
(cc) L45Q on the VH, and A43Q and Y49A on the VL;
(dd) L45Q on the VH, and P44A and Y49A on the VL;
(ee) L45Q on the VH, and L46Q and Y49A on the VL;
(ff) L45Q on the VH, and Y49A and Y87L on the VL;
(gg) L45Q on the VH, and Y49A and F98M on the VL;
(hh) Y91M on the VH, and A43Q, P44A, and Y49A on the VL;
(ii) Y91M on the VH, and A43Q, L46Q, and Y49A on the VL;
(jj) Y91M on the VH, and L46Q, Y49A, and Y87M on the VL;
(kk) V37S on the VH, and L46Q, Y49A, and Y87M on the VL;
(ll) V37S and L45Q on the VH, and A43Q and Y49A on the VL;
(mm) V37S and Y91M on the VH, and A43Q and Y49A on the VL;
(nn) V37S and W103M on the VH, and A43Q and Y49A on the VL;
(oo) V37S and Y91M on the VH, and L46Q and Y49A on the VL; and
(pp) V37S and L45Q on the VH, and Y49A and Y87M on the VL.

In one embodiment, the amino acid modification is a substitution of an amino acid present at the interface between the VH and the VL within the FR region. In a preferable embodiment, the substitutions are selected from positions 37, 45, 91 and 103 on the VH and positions 43, 46, 49 and 87 on the VL, particularly, the substitutions are V37S, L45Q, Y91M, Y91A, H91A, W103L, W103I and W103M on the VH and A43Q, L46Q, Y49A and Y87L on the VL (according to Kabat numbering).

As described herein, amino acid residue positions involved in the association of VH and VL mainly residing in framework regions (FRs) are known in the art, and are considered to be relatively conserved across antibodies, such as for example, within the IgG class of antibodies. Modifications in these regions would be reasonably expected by those skilled in the art to alter the association of VH and VL. Those skilled in the art may readily increase or decrease the association of VH and VL by performing one or more amino acid modifications at these selected residue positions, such as for example, to reduce the association of VH and VL, the skilled person in the art may introduce hydrophobic residues into a hydrophilic region, or introduce hydrophilic residues into a hydrophobic pocket, or modify a hydrophobic/hydrophilic residue to one that is neutral. Since the amino acid residue at these positions are highly conserved, the introduction of variant amino acids at the selected positions will generally affect the VH/VL association in an antibody comprising said FRs, regardless of its target antigen.

In one embodiment, amino acid residues residing in the interface between the ligand-binding domain and the ligand or in the interface between the antigen binding domain and the antigen, i.e. residing within the CDRs, is additionally altered to promote the dissociation of, or reduce the interaction or association between the VH and VL as approximately 25% of amino acid residues that are crucial in maintaining the association between VH and VL resides within the CDRs. In order to preserve the binding of the ligand or antigen to the ligand-binding domain or antigen-binding domain, amino acid positions selected for modification must not disrupt the binding of the ligand or antigen to the ligand-binding domain or antigen-binding domain such that the biological activity of the ligand or antigen is attenuated, i.e. not capable of exerting its biological activity in the absence of protease. Screening for suitable amino acid positions within the CDRs for modification as described above can be conducted by any of the common methods described herein or as known to the skilled person. In some embodiments, amino acid modification involves substitution of at least one amino acid present at the interface between the ligand-binding domain and the ligand or at the interface between the antigen binding domain and the antigen. In a preferable embodiment, the at least one substitution includes positions 30 and 100a, particularly, the substitution is selected from S30V and F100aI.

In one embodiment, the amino acid modification are combinations of substitution(s) of (an) amino acid(s) present at the interface between the VH and the VL within the FR region and optionally within the CDR region. In a preferable embodiment, the combination substitutions are selected from the following groups (a) to (cc):
(a) L46Q and Y49A on the VL;
(b) Y91A on the VH, and L46Q and Y49A on the VL;
(c) Y91M on the VH, and A43Q and Y49A on the VL;
(d) Y91M on the VH, and A43Q, L46Q, and Y49A on the VL;
(e) H91A on the VH, and L46Q and Y49A on the VL;
(f) W103L on the VH, and L46Q and Y49A on the VL;
(g) W103I on the VH, and L46Q and Y49A on the VL;
(h) W103M on the VH, and A43Q and Y49A on the VL;
(i) W103M on the VH, and L46Q and Y49A on the VL;
(j) V37S on the VH, and A43Q and Y49A on the VL;
(k) V37S on the VH, and L46Q and Y49A on the VL;
(l) L45Q on the VH, and A43Q and Y49A on the VL;
(m) L45Q on the VH, and L46Q and Y49A on the VL;
(n) F100aI on the VH, and A43Q and Y49A on the VL;
(o) F100aI on the VH, and A43Q, L46Q, and Y49A on the VL;
(p) W103L on the VH, and S30V, L46Q, and Y49A on the VL;
(q) W103M on the VH, and S30V, L46Q, and Y49A on the VL;
(r) V37S and F100aI on the VH, and S30V, A43Q, and Y49A on the VL;
(s) V37S and F100aI on the VH, and S30V, L46Q, and Y49A on the VL;
(t) W103L on the VH, and L46Q and Y49A on the VL;
(u) W103I on the VH, and L46Q and Y49A on the VL;
(v) W103M on the VH, and Y49A and Y87L on the VL;
(w) W103L on the VH, and Y49A and Y87L on the VL;
(x) W103L on the VH, and S30V, Y49A, and Y87L on the VL;
(y) V37S and F100aI on the VH, and L46Q and Y49A on the VL;
(z) V37S and F100aI on the VH, and Y49A and Y87L on the VL;
(aa) V37S and F100aI on the VH, and S30V, Y49A, and Y87L on the VL;
(bb) V37S, F100aI and W103M on the VH, and L46Q and Y49A on the VL; and
(cc) V37S, F100aI and W103L on the VH, and L46Q and Y49A on the VL.

In the present specification, the one or more amino acid modifications performed at the interface between VH and VL, or optionally including modifications performed at the interface between the ligand-binding domain and the ligand or in the interface between the antigen binding domain and the antigen, as identified above, may reduce the association between VH and VL, and promotes the dissociation of VH or VL, or VH-ligand, or VL-ligand, from the fusion protein comprising a ligand-binding domain or the IgG antibody-like polypeptide comprising an antigen-binding domain of the present specification, wherein said fusion protein or IgG-like polypeptide comprises a protease cleavage site cleavable in the presence of a protease. The present specification includes IL-12 and IL-22 bivalent fusion proteins as described in any of the embodiments herein, and also other bivalent fusion proteins as described in the following patent applications: WO2018097307, WO2018097308, WO2019107380, WO2019107384, WO2019230866, WO2019230867, WO2019230868, WO2020116498, and WO2021149697. Without wishing to be bound by theory, the amino acid modifications as described herein would affect the VH/VL association in an IgG antibody-like polypeptide comprising an antigen-binding domain or a fusion protein comprising the ligand-binding domain of the present specification, irrespective of antigen or ligand identity, due to the highly conserved nature of the identified amino acid positions which mainly occur in the FRs, in particular, wherein the IgG antibody-like polypeptides or fusion proteins comprises the same or similar framework regions as the IgG antibody-like polypeptides or fusion proteins of the present specification. To identify if the one or more amino acid modification(s) at the interface between VH and VL, and additionally modifications performed at the interface between the ligand-binding domain and the ligand or in the interface between the antigen binding domain and the antigen is sufficient to promote dissociation from each other and yet able to maintain binding to its target antigen or ligand, the skilled person may employ any methods known in the art or as described herein.

It can be verified that the amino acid modification(s) introduced at selected position(s) within the interface between VH and VL reduces association between VH and VL to a level sufficient to promote dissociation from each other using methods known to the skilled person. A method of detecting the release of VH or VL from the fusion protein or antibody or antibody fragment includes a method of detecting VH(s) or VL(s) release by comparing the molecular weight of the fusion protein, antibody or antibody fragment before and after protease cleavage using well-known methods such as SDS-PAGE, Size Exclusion Chromatography (SEC) or BIACORE using SPR. In an assay using BIACORE, the fusion protein, antibody or antibody fragment is immobilised on R-Protein A coupled—carboxymethylated dextran biosensor chips (CM4-ProA/G, BIACORE, Inc.) biosensor chips that are activated with N-ethyl-N′-(3-dimethylaminopropyl)-carbodiimide hydrochloride (EDC) and N-hydroxysuccinimide (NHS) according to the supplier's instructions. Protease, such as urokinase-type plasminogen activator (uPA), at a concentration of 400 nM is injected in assay buffer (HBS-EP+, Cytiva) at a flow rate of 2 microliter/min at 37 degrees C. for an association time of 1800 secs and dissociation time of 10 secs. The response unit (RU) captured before and after protease injection is compared. A percentage reduction in response unit of less than or equivalent to 1%, or less than or equivalent to 2%, or less than or equivalent to 3%, or less than or equivalent to 4%, or less than or equivalent to 5%, or less than or equivalent to 6%, or less than or equivalent to 7% or less than or equivalent to 8%, or less than or equivalent to 9%, or less than or equivalent to 10%, or less than or equivalent to 11%, or less than or equivalent to 12%, or less than or equivalent to 13%, or less than or equivalent to 14%, or less than or equivalent to 15%, or less than or equivalent to 16%, or less than or equivalent to 17%, or less than or equivalent to 18%, or less than or equivalent to 19%, or less than or equivalent to 20%, or less than or equivalent to 21%, or less than or equivalent to 22%, or less than or equivalent to 23%, or less than or equivalent to 24%, or less than or equivalent to 25%, or less than or equivalent to 26%, or less than or equivalent to 27%, or less than or equivalent to 28%, or less than or equivalent to 29%, or less than or equivalent to 30%, or less than or equivalent to 31%, or less than or equivalent to 32%, or less than or equivalent to 33%, or less than or equivalent to 34%, or less than or equivalent to 35%, or less than or equivalent to 36%, or less than or equivalent to 37%, or less than or equivalent to 38%, or less than or equivalent to 39%, or less than or equivalent to 40%, before and after protease cleavage indicates dissociation of a fragment from the fusion protein, antibody or antibody fragment. In a preferred embodiment, a dissociation of less than or equivalent to 10% indicates complete dissociation of VH(s) or VL(s) from a bivalent fusion polypeptide of the present invention. In an alternative embodiment, a dissociation of less than or equivalent to 20% indicates complete dissociation of VH(s) or VL(s) from an IgG-like antibody molecule or IgG antibody of the present invention. In an alternative embodiment, a dissociation of less than or equivalent to 15.8% indicates complete dissociation of VH(s) or VL(s) from a bivalent fusion polypeptide of the present invention. In an alternative embodiment, a dissociation of less than or equivalent to 36.8% indicates complete dissociation of VH-ligand or VL-ligand from a bivalent fusion polypeptide of the present invention. In an alternative embodiment, a dissociation of less than or equivalent to 30% indicates complete dissociation of VH-ligand or VL-ligand from a bivalent fusion polypeptide of the present invention.

Without wishing to be bound by any theory, it is expected that the aforementioned amino acid modifications at the interface between VH and VL will promote dissociation of VH from VL, or vice versa, in any molecular form comprising a VH that associates with a VL. To this end, the described amino acid modifications at the interface between VH and VL are applicable to the above described bivalent homodimer fusion protein comprising a ligand-binding domain, bivalent homodimer fusion protein comprising an IgG antibody-like polypeptide comprising an antigen-binding domain, bivalent homodimer fusion protein comprising a full-length IgG antibody comprising an antigen-binding domain, polypeptide comprising an antigen-binding domain, antibody comprising an antigen-binding domain and antibody fragment comprising an antigen-binding domain.

In the instance of the bivalent homodimer fusion protein comprising a ligand-binding domain, upon protease cleavage, the release of the VH disrupts the binding of the ligand to the ligand-binding domain of the ligand-binding moiety/molecule, allowing the ligand to freely dissociate and bind to its receptor, thereby activating receptor signalling. In the instance of the bivalent homodimer fusion protein comprising an IgG antibody-like polypeptide comprising an antigen-binding domain, upon protease cleavage, the release of the VH disrupts the binding of the ligand to the antigen-binding domain of the antigen variable region, allowing the ligand to freely dissociate and bind to its receptor, thereby activating receptor signalling. In some embodiments of the above instances, the ligand freely binds to the receptor while being bound to the C-terminal Fc region of the ligand-binding moiety/molecule, or the antibody constant region. In the instance of the bivalent homodimer fusion protein comprising a full-length IgG antibody comprising an antigen-binding domain, polypeptide comprising an antigen-binding domain or antibody comprising an antigen-binding domain, upon protease cleavage, the release of the VH disrupts the binding of the ligand to the variable region, allowing the ligand to freely dissociate and bind to its receptor, thereby activating receptor signalling. In the instance of an antibody fragment comprising an antigen-binding domain, upon protease cleavage, there is reduced association between a portion of the VH and a portion of the VL, resulting in the complete dissociation of VH and VL. This dissociation disrupts the binding of the ligand to the antigen-binding domain of the antibody fragment, allowing the ligand to freely dissociate and bind to its receptor, thereby activating receptor signalling. In some embodiments, upon protease cleavage, the ligand dissociates from the antigen-binding domain or variable region and freely binds to its receptor while being bound to the N-terminal variable domain, either a VH or a VL. In some embodiments, upon protease cleavage, the ligand dissociates from the ligand-binding domain and freely binds to its receptor while being bound to the C-terminal Fc region of the ligand-binding moiety/molecule or the constant region. It is further contemplated that the above described amino acid modifications between the interface of VH and VL in any molecular form, including antibody fragments, will promote dissociation of one from the other, as long as said molecular form comprises a VH and a VL associating with each other. Hence, the present invention includes methods of producing molecular forms comprising such amino acid modifications and uses of any of the abovementioned molecular forms in a method of promoting the dissociation of VH and VL thereof. Moreover, those skilled in the art with reference to the present invention, may change the form of a molecule comprising antibody VH, antibody VL, and optionally an antibody constant region, for example, by swapping the antibody VH with the antibody VL. Such a molecular form does not depart from the scope of the present invention.

### Methods of Screening

The present invention also include a method of screening to identify amino acid modifications in the above described molecular formats, including the bivalent homodimer fusion protein comprising a ligand-binding domain, the bivalent homodimer fusion protein comprising an IgG antibody-like polypeptide comprising an antigen-binding domain, the bivalent homodimer fusion protein comprising a full-length IgG antibody comprising an antigen-binding domain, the polypeptide, antibody or antibody fragment comprising an antigen-binding domain, comprising the steps:
(a) introducing at least one amino acid mutation or at least one pair of amino acid mutation at the interface between VH and VL in said fusion protein or polypeptide, and optionally introducing at least one amino acid mutation at the interface between the ligand or antigen and the ligand-binding domain or antigen-binding domain, that promote dissociation of VH domain or VL domain from the fusion protein or polypeptide;
(b) determining a first response unit (RU1) of immobilised fusion protein or polypeptide of step (a) in a BIACORE surface plasma resonance (SPR) assay in the absence of protease;
(b) determining a second response unit (RU2) of immobilised fusion protein or polypeptide of step (a) in the same BIACORE surface plasma resonance (SPR) assay in the presence of protease;
(c) selecting the mutation(s) in step (a) if the percentage difference between RU1 and RU2 is less than or equivalent to 1%, or is less than or equivalent to 2%, or is less than or equivalent to 3%, or is less than or equivalent to 4%, or is less than or equivalent to 5%, or is less than or equivalent to 6%, or is less than or equivalent to 7%, or is less than or equivalent to 8%, or is less than or equivalent to 9%, or is less than or equivalent to 10%, or is less than or equivalent to 11%, or is less than or equivalent to 12%, or is less than or equivalent to 13%, or is less than or equivalent to 14%, or is less than or equivalent to 15%, or is less than or equivalent to 16%, or is less than or equivalent to 17%, or is less than or equivalent to 18%, or is less than or equivalent to 19%, or is less than or equivalent to 20%, or is less than or equivalent to 21%, or is less than or equivalent to 22%, or is less than or equivalent to 23%, or is less than or equivalent to 24%, or is less than or equivalent to 25%, or is less than or equivalent to 26%, or is less than or equivalent to 27%, or is less than or equivalent to 28%, or is less than or equivalent to 29%, or is less than or equivalent to 30%, or is less than or equivalent to 31%, or is less than or equivalent to 32%, or is less than or equivalent to 33%, or is less than or equivalent to 34%, or is less than or equivalent to 35%, or is less than or equivalent to 36%, or is less than or equivalent to 37%, or is less than or equivalent to 38%, or is less than or equivalent to 39%, or is less than or equivalent to 40%, before and after protease cleavage;
wherein the percentage reduction in response unit (RU) corresponds to the percentage reduction in molecular weight resulting from the release of VH or VL from the fusion protein or polypeptide.

The present invention also include a method for screening for a fusion protein, polypeptide, antibody or antibody fragment as described in any of the above embodiments, having mutations that reduce association between VH and VL, comprising comparing the maximum response unit recorded for any of the above fusion protein before and after protease cleavage under surface plasma resonance (SPR) and selecting mutations that result in a reduction in response unit of less than or equivalent to 1%, or less than or equivalent to 2%, or less than or equivalent to 3%, or less than or equivalent to 4%, or less than or equivalent to 5%, or less than or equivalent to 6%, or less than or equivalent to 7%, or less than or equivalent to 8%, or less than or equivalent to 9%, or less than or equivalent to 10%, or less than or equivalent to 11%, or less than or equivalent to 12%, or less than or equivalent to 13%, or less than or equivalent to 14%, or less than or equivalent to 15%, or less than or equivalent to 16%, or less than or equivalent to 17%, or less than or equivalent to 18%, or less than or equivalent to 19%, or less than or equivalent to 20% or less than or equivalent to 21%, or less than or equivalent to 22%, or less than or equivalent to 23%, or less than or equivalent to 24%, or less than or equivalent to 25%, or less than or equivalent to 26%, or less than or equivalent to 27%, or less than or equivalent to 28%, or less than or equivalent to 29%, or less than or equivalent to 30%, or less than or equivalent to 31%, or less than or equivalent to 32%, or less than or equivalent to 33%, or less than or equivalent to 34%, or less than or equivalent to 35%, or less than or equivalent to 36%, or less than or equivalent to 37%, or less than or equivalent to 38%, or less than or equivalent to 39%, or less than or equivalent to 40%, before and after protease cleavage.

In some embodiments, the percentage reduction in response unit corresponding to less than or equivalent to 1%, or less than or equivalent to 2%, or less than or equivalent to 3%, or less than or equivalent to 4%, or less than or equivalent to 5%, or less than or equivalent to 6%, or less than or equivalent to 7%, or less than or equivalent to 8%, or less than or equivalent to 9%, or less than or equivalent to 10% corresponds to dissociation of VH or VL from the fusion protein, polypeptide, antibody or antibody fragment as described in any of the above embodiments. In some embodiments, the percentage reduction in response unit corresponding to less than or equivalent to 1%, or less than or equivalent to 2%, or less than or equivalent to 3%, or less than or equivalent to 4%, or less than or equivalent to 5%, or less than or equivalent to 6%, or less than or equivalent to 7%, or less than or equivalent to 8%, or less than or equivalent to 9%, or less than or equivalent to 10%, or less than or equivalent to 11%, or less than or equivalent to 12%, or less than or equivalent to 13%, or less than or equivalent to 14%, or less than or equivalent to 15%, or less than or equivalent to 16%, or less than or equivalent to 17%, or less than or equivalent to 18%, or less than or equivalent to 19%, or less than or equivalent to 20% corresponds to dissociation of VH or VL from the fusion protein, polypeptide, antibody or antibody fragment as described in any of the above embodiments. In a preferred embodiment, a dissociation of less than or equivalent to 10% indicates complete dissociation of VH(s) or VL(s) from a bivalent fusion polypeptide of the present invention. In an alternative embodiment, a dissociation of less than or equivalent to 20% indicates complete dissociation of VH(s) or VL(s) from an IgG-like antibody molecule or IgG antibody of the present invention. In an alternative, a dissociation of less than or equivalent 15.8% indicates complete dissociation of VH(s) or VL(s) from a bivalent fusion polypeptide of the present invention. In an alternative embodiment, a dissociation of less than or equivalent to 36.8% indicates complete dissociation of VH-ligand or VL-ligand from a bivalent fusion polypeptide of the present invention. In an alternative embodiment, a dissociation of less than or equivalent to 30% indicates complete dissociation of VH-ligand or VL-ligand from a bivalent fusion polypeptide of the present invention.

The present invention also includes a method for screening for a fusion protein, polypeptide, antibody or antibody fragment as described in any of the above embodiments, having mutations that reduce association between VH and VL, comprising the steps of:
(a) introducing at least one amino acid mutation or at least one pair of amino acid mutation at the interface between VH and VL in said fusion protein or polypeptide, and optionally introducing at least one amino acid mutation at the interface between the ligand or antigen and the ligand-binding domain or antigen-binding domain, that promote dissociation of VH domain or VL domain from the fusion protein or polypeptide;
(b) subjecting a first set of the fusion protein or polypeptide before protease cleavage to Size Exclusion Chromatography (SEC) and obtaining a first chromatograph comprising peak A1 (a first peak);
(c) subjecting a second set of the fusion protein or polypeptide after protease cleavage to SEC and obtaining a second chromatograph comprising peak A2 (a second peak) and additional peak A2′ (a third peak), wherein A2′ is a shoulder peak of A2;
(d) determining the percentage resulting from area under curve (AUC) of peak A2′ (the third peak) over AUC of peak A1 (the first peak);
(e) selecting the mutation(s) in step (a) wherein percentage obtained in step (d) corresponds to less than or equivalent to 1%, or less than or equivalent to 2%, or less than or equivalent to 3%, or less than or equivalent to 4%, or less than or equivalent to 5%, or less than or equivalent to 6%, or less than or equivalent to 7%, or less than or equivalent to 8%, or less than or equivalent to 9%, or less than or equivalent to 10%, or less than or equivalent to 11%, or less than or equivalent to 12%, or less than or equivalent to 13%, or less than or equivalent to 14%, or less than or equivalent to 15%, or less than or equivalent to 16%, or less than or equivalent to 17%, or less than or equivalent to 18%, or less than or equivalent to 19%, or less than or equivalent to 20%, or less than or equivalent to 21%, or less than or equivalent to 22%, or less than or equivalent to 23%, or less than or equivalent to 24%, or less than or equivalent to 25%, or less than or equivalent to 26%, or less than or equivalent to 27%, or less than or equivalent to 28%, or less than or equivalent to 29%, or less than or equivalent to 30%, or less than or equivalent to 31%, or less than or equivalent to 32%, or less than or equivalent to 33%, or less than or equivalent to 34%, or less than or equivalent to 35%, or less than or equivalent to 36% or less than or equivalent to 37%, or less than or equivalent to 38%, or less than or equivalent to 39%, or less than or equivalent to 40%; wherein the percentage determined in (d) corresponds to the percentage of VH domain or VL domain dissociated from the fusion protein or polypeptide after protease cleavage.

In some embodiments, the percentage determined in step (d) in response unit corresponding to is less than or equivalent to 1%, or is less than or equivalent to 2%, or is less than or equivalent to 3%, or is less than or equivalent to 4%, or is less than or equivalent to 5%, or is less than or equivalent to 6%, or is less than or equivalent to 7%, or is less than or equivalent to 8%, or is less than or equivalent to 9%, or is less than or equivalent to 10% corresponds to dissociation of VH or VL from the fusion protein, polypeptide, antibody or antibody fragment as described in any of the above embodiments. In some embodiments, the percentage determined in step (d) in response unit corresponding to is less than or equivalent to 1%, or is less than or equivalent to 2%, or is less than or equivalent to 3%, or is less than or equivalent to 4%, or is less than or equivalent to 5%, or is less than or equivalent to 6%, or is less than or equivalent to 7%, or is less than or equivalent to 8%, or is less than or equivalent to 9%, or is less than or equivalent to 10%, or is less than or equivalent to 11%, or is less than or equivalent to 12%, or is less than or equivalent to 13%, or is less than or equivalent to 14%, or is less than or equivalent to 15%, or is less than or equivalent to 16%, or is less than or equivalent to 17%, or is less than or equivalent to 18%, or is less than or equivalent to 19%, or is less than or equivalent to 20%, or is less than or equivalent to 21%, or is less than or equivalent to 22%, or is less than or equivalent to 23%, or is less than or equivalent to 24%, or is less than or equivalent to 25%, or is less than or equivalent to 26%, or is less than or equivalent to 27%, or is less than or equivalent to 28%, or is less than or equivalent to 29%, or is less than or equivalent to 30%, or is less than or equivalent to 31%, or is less than or equivalent to 32%, or is less than or equivalent to 33%, or is less than or equivalent to 34%, or is less than or equivalent to 35%, corresponds to dissociation of VH or VL from the fusion protein, polypeptide, antibody or antibody fragment as described in any of the above embodiments. In a preferred embodiment, a dissociation is less than or equivalent to, or of about 10% indicates complete dissociation of VH(s) or VL(s) from a bivalent fusion polypeptide of the present invention. In an alternative embodiment, a dissociation is less than or equivalent to, or of about 20% indicates complete dissociation of VH(s) or VL(s) from an IgG-like antibody molecule or IgG antibody of the present invention. In an alternative embodiment, a dissociation is less than or equivalent to, or of about 15.8% indicates complete dissociation of VH(s) or VL(s) from a bivalent fusion polypeptide of the present invention. In an alternative embodiment, a dissociation is less than or equivalent to, or of about 36.8% indicates complete dissociation of VH-ligand or VL-ligand from a bivalent fusion polypeptide of the present invention. In an alternative embodiment, a dissociation is less than or equivalent to, or of about 30% indicates complete dissociation of VH-ligand or VL-ligand from a bivalent fusion polypeptide of the present invention.

The methods of screening described herein further includes a step of verification of the biological activity of the fusion protein or polypeptide of the present specification in the first state and in the second state, i.e. before and after protease cleavage. The step of verification of biological activity includes biological assays described herein, or as detailed in the Examples, or suitably employed by the skilled person, to evaluate the biological activity of the ligand or antigen before and after protease cleavage.

The methods of screening described herein further comprising the following step(s): wherein for a given concentration of ligand or antigen and a concentration of fusion protein or polypeptide of the present specification corresponding to said concentration of ligand or antigen;
I. determining the biological activity of a fusion protein or polypeptide before protease cleavage;
II. determining the biological activity of the fusion protein or polypeptide in step I after protease cleavage;
III. introducing at least one amino acid modification or at least one pair of amino acid modification at the interface between VH and VL in the fusion protein or polypeptide in step I, and optionally introducing at least one amino acid modification at the interface between the ligand or antigen and the ligand-binding domain or antigen-binding domain, wherein the amino acid modification(s) promote dissociation of VH domain or VL domain from the fusion protein or polypeptide upon protease cleavage in the presence of a protease;
IV. determining the biological activity of the fusion protein or polypeptide in step III, before protease cleavage;
V. determining the biological activity of the fusion protein or polypeptide in step III, after protease cleavage;
VI. selecting amino acid modification(s) wherein the biological activity of the fusion protein or polypeptide of step (IV) is less than the biological activity of the fusion protein or polypeptide of step (V);
VII. selecting amino acid modification(s) wherein the biological activity of the fusion protein or polypeptide in step (V) is greater than the biological activity of the fusion protein or polypeptide in step (IV), and/or
VIII. determining the difference in biological activity of the biological activity of the fusion protein or polypeptide between (I) and (II), “VI”, and the difference in biological activity of the biological activity of the fusion protein or polypeptide between (IV) and (V), “V2”; and
IX. selecting the amino acid modification(s) wherein the value of V2 is greater than V1.

For the avoidance of doubt, the amino acid modification(s) capable of being introduced in step III includes any of the modifications that reduce association between VH and VL in a fusion protein, IgG antibody-like polypeptide, antibody or antibody fragment as described in any of the above embodiments, or derived from following the steps (a), and/or (b), and/or (c), and/or (d), and/or (e) set out above. Further, determination of whether a given concentration of ligand or antigen and a concentration of fusion protein or IgG antibody-like polypeptide of the present specification corresponds to the same concentration as that of its corresponding ligand or antigen can be readily determined by performing suitable biological assays that assess the biological activity of the fusion protein or IgG antibody-like polypeptide, as detailed in the Examples or as described in any of the above embodiments.

The present invention further includes a library of amino acid modifications that reduce association between VH and VL in a fusion protein, polypeptide, antibody or antibody fragment as described in any of the above embodiments comprising the amino acid modifications at the interface between VH and VL. The library includes any of amino acid substitutions selected from positions Q39D, W47A, W47L, W47M, Y91A, Y91L, Y91M, H91A, W103A, W103L, W103M, V37S, V37Q, G44Q, L45A, and L45Q on the VH, or R38E, Y49A, Y87A, Y87L, Y87M, F98A, F98L, F98M, A43Q, P44A, P44S, P44Q, L46E, and L46Q on the VL (according to Kabat numbering). The library can include selection of at least one, two, three, four, five etc. amino acid substitutions selected from positions Q39D, W47A, W47L, W47M, Y91A, Y91L, Y91M, H91A, W103A, W103L, W103M, V37S, V37Q, G44Q, L45A, and L45Q on the VH and/or a corresponding at least one, two, three, four, five etc. amino acid substitutions selected from positions R38E, Y49A, Y87A, Y87L, Y87M, F98A, F98L, F98M, A43Q, P44A, P44S, P44Q, L46E, and L46Q on the VL (according to Kabat numbering). In a further aspect of the invention, the library includes at least one amino acid substitution selected from at positions 30 and 100a (according to Kabat numbering). In a further aspect, the substitution is selected from S30V and F100aI. It should be noted that the library is not limited to aforementioned substitutions but include without limitation any substitutions that may be derived from following the steps set out in the methods of screening described herein, e.g., in this “Methods of screening” section herein.

### Ligand

In the present specification, the term “ligand moiety” or “ligand molecule” refers to a moiety or molecule having biological activity. Herein, the “ligand moiety” and “ligand molecule” may be simply referred to as “ligand”. The molecule having biological activity usually functions by interacting with a receptor on cell surface and thereby performing biological stimulation, inhibition, or modulation in other modes. These functions are usually thought to participate in the intracellular signalling pathways of cells carrying the receptor.

In the present specification, the ligand encompasses the desired molecule that exerts biological activity through interaction with a biomolecule, also referred herein as “binding partner”. For example, the ligand not only means a molecule that interacts with a receptor but also includes a molecule that exerts biological activity through interaction with the molecule, for example, a receptor that interacts with the molecule, or a binding fragment thereof. For example, a ligand binding site of a protein known as a receptor, and a protein containing an interaction site of the receptor with another molecule are included in the ligand according to the present invention. Specifically, for example, a soluble receptor, a soluble fragment of a receptor, an extracellular domain of a transmembrane receptor, and polypeptides containing them are included in the ligand according to the present invention.

The ligand of the present invention can usually exert desirable biological activity by binding to one or more binding partners. The binding partner of the ligand can be an extracellular, intracellular, or transmembrane protein. In one embodiment, the binding partner of the ligand is an extracellular protein, for example, a soluble receptor. In another embodiment, the binding partner of the ligand is a membrane-bound receptor. The ligand of the present invention can specifically bind to the binding partner with a dissociation constant (KD) of 10 micromolar (micro M), 1 micromolar, 100 nM, 50 nM, 10 nM, 5 nM, 1 nM, 500 pM, 400 pM, 350 pM, 300 pM, 250 pM, 200 pM, 150 pM, 100 pM, 50 pM, 25 pM, 10 pM, 5 pM, 1 pM, 0.5 pM, or 0.1 pM or less.

Examples of the molecule having biological activity include, but are not limited to, cytokines, chemokines, polypeptide hormones, growth factors, apoptosis inducing factors, PAMPs, DAMPs, nucleic acids, and fragments thereof. In a specific embodiment, an interleukin, an interferon, a hematopoietic factor, a member of the TNF superfamily, a chemokine, a cell growth factor, a member of the TGF-beta family, a myokine, an adipokine, or a neurotrophic factor can be used as the ligand. In a more specific embodiment, CXCL9, CXCL10, CXCL11, IL-2, IL-7, IL-12, IL-15, IL-18, IL-21, IL-22, IFN-alpha, IFN-beta, IFN-g, MIG, I-TAC, RANTES, MIP-1a, MIP-1b, IL-1R1 (Interleukin-1 receptor, type I), IL-1R2 (Interleukin-1 receptor, type II), IL-1RAcP (Interleukin-1 receptor accessory protein), or IL-1Ra (Protein Accession No. NP_776214, mRNA Accession No. NM_173842.2) can be used as the ligand. There is no limitation on the ligand used in the present disclosure. In some embodiments, the ligand may be a wild-type (or naturally occurring) ligand or a mutant ligand with any mutation(s). In the case of IL-12 or IL-22 which is a heterodimeric cytokine, in some embodiments, the ligand, IL-12, may be wild-type (or naturally-occurring) IL-12 or IL-22, or a mutant IL-12 or IL-22 with any mutation(s). In some embodiments, IL-12 may be a single-chain IL-12 in which p35 and p40 are linked to be contained in a single chain.

In some embodiments of the present invention, the ligand is a cytokine. Cytokines are a secreted cell signalling protein family involved in immunomodulatory and inflammatory processes. These cytokines are secreted by glial cells of the nervous system and by many cells of the immune system. The cytokines can be classified into proteins, peptides and glycoproteins and encompass large diverse regulator families. The cytokines can induce intracellular signal transduction through binding to their cell surface receptors, thereby causing the regulation of enzyme activity, upregulation or downregulation of some genes and transcriptional factors thereof, or feedback inhibition, etc.

In some embodiments, the cytokine of the present invention includes immunomodulatory factors such as interleukins (IL) and interferons (IFN). A suitable cytokine can contain a protein derived from one or more of the following types: four alpha-helix bundle families (which include the IL-2 subfamily, the IFN subfamily and IL-10 subfamily); the IL-1 family (which includes IL-1 and IL-8); and the IL-17 family. The cytokine can also include those classified into type 1 cytokines (e.g., IFNgamma and TGF-beta) which enhance cellular immune response, or type 2 cytokines (e.g., IL-4, IL-10, and IL-13) which work advantageously for antibody reaction.

Interleukin 12 (IL-12) is a heterodimeric cytokine consisting of disulphide-linked glycosylated polypeptide chains of 30 and 40 kD (Accession No. NP_000873.2, P29459 and Accession No. NP_002178.2, P29460). Cytokines are synthesized and then secreted by dendritic cells, monocytes, macrophages, B cells, Langerhans cells and keratinocytes, and antigen-presenting cells including natural killer (NK) cells. IL-12 mediates various biological processes and has been mentioned as a NK cell stimulatory factor (NKSF), a T cell stimulatory factor, a cytotoxic T lymphocyte maturation factor and an EBV-transformed B cell line factor.

Interleukin 12 can bind to an IL-12 receptor expressed on the cytoplasmic membranes of cells (e.g., T cells and NK cells) and thereby change (e.g., start or block) a biological process. For example, the binding of IL-12 to an IL-12 receptor stimulates the growth of preactivated T cells and NK cells, promotes the cytolytic activity of cytotoxic T cells (CTL), NK cells and LAK (lymphokine-activated killer) cells, induces the production of gamma interferon (IFNgamma) by T cells and NK cells, and induces the differentiation of naive Th0 cells into Th1 cells producing IFNgamma and IL-2. In particular, IL-12 is absolutely necessary for setting the production and cellular immune response (e.g., Th1 cell-mediated immune response) of cytolytic cells (e.g., NK and CTL). Thus, IL-12 is absolutely necessary for generating and regulating both protective immunity (e.g., eradication of infectious disease) and pathological immune response (e.g., autoimmunity).

Examples of the method for measuring the physiological activity of IL-12 include a method of measuring the cell growth activity of IL-12, STAT4 reporter assay, a method of measuring cell activation (cell surface marker expression, cytokine production, etc.) by IL-12, and a method of measuring the promotion of cell differentiation by IL-12.

Interleukin 22 (IL-22) (Accession No. NP_065386.1, Q9GZX6) is a member of the IL-10 family of cytokines. It is secreted by immune cells such as T-cells, NKT-cells, type 3 innate lymphoid cells (ILC3), and to a lesser extent by neutrophils and macrophages. IL-22 binds to its receptor IL-22R, which is a heterodimer composed of IL-22R1 and IL-10R2. IL22R is mainly expressed on non-hematopoetic cells such as epithelial cells and stromal cells. IL-22 activity is regulated by IL-22 binding protein (IL-22BP, also known as IL22RA2), which is a secreted protein with high structural homology to IL-22R1. IL-22BP binds to IL-22 with high affinity, blocking it from interacting with IL-22R1.

Binding of IL-22 to IL-22 receptor leads to activation of JAK1 and TYK2 kinases, which in turn leads to activation of STAT3 signaling. IL-22 plays an important role in epithelial cell function. For example, in the gut, IL-22 promotes the integrity of the intestinal barrier by stimulating proliferation of gut epithelial cells, mucus secretion and anti-microbial peptide secretion. In the liver, IL-22 acts as a survival factor for hepatocytes during liver injury, and also stimulates hepatocytes to proliferate for liver regeneration.

Examples of the method for measuring the physiological activity of IL-22 include a method of measuring the cell growth activity of IL-22, STAT3 reporter assay, and a method of measuring cell activation (cell surface marker expression, cytokine production, etc.) by IL-22.

Interleukin 2 (IL-2) is monomeric cytokine and mainly secreted by activated CD4 T and CD8 T cells. IL-2 binds to its receptor (IL-2R), which consists of 3 subunits, alpha, beta, and gamma. IL-2R beta and gamma are involved in signal transduction and IL-2R alpha and beta are involved in binding. All three subunits are important for high affinity cytokine-receptor complex. IL-2 is essential for both promoting and regulating immune responses since it binds and activates both effector T cells and regulatory T cells.

Examples of the method for measuring the physiological activity of IL-2 include a method of measuring the cell growth activity of IL-2, a method of measuring cell activation (cell surface marker expression, cytokine production, etc.) by IL-2, and a method of measuring the promotion of cell differentiation by IL-2.

In some embodiments of the present invention, the ligand is a chemokine. Chemokines generally act as chemoattractants that mobilize immune effector cells to chemokine expression sites. This is considered beneficial for expressing a particular chemokine gene, for example, together with a cytokine gene, for the purpose of mobilizing other immune system components to a treatment site. Such chemokines include CXCL10, RANTES, MCAF, MIP1-alpha, and MIP1-beta. Those skilled in the art should know that certain cytokines also have a chemoattractive effect and acknowledge that such cytokines can be classified by the term “chemokine”.

Chemokines are a homogeneous serum protein family of 7 to 16 kDa originally characterized by their ability to induce leukocyte migration. Most of chemokines have four characteristic cysteines (Cys) and are classified into CXC or alpha, CC or beta, C or gamma and CX3C or delta chemokine classes according to motifs formed by the first two cysteines. Two disulphide bonds are formed between the first and third cysteines and between the second and fourth cysteines. In general, the disulphide bridges are considered necessary. Clark-Lewis and collaborators have reported that the disulphide bonds are crucial for the chemokine activity of at least CXCL10 (Clark-Lewis et al., J. Biol. Chem. 269: 16075-16081, 1994). The only one exception to having four cysteines is lymphotactin, which has only two cysteine residues. Thus, lymphotactin narrowly maintains its functional structure by only one disulphide bond. Subfamilies of CXC or alpha are further classified, according to the presence of an ELR motif (Glu-Leu-Arg) preceding the first cysteine, into two groups: ELR-CXC chemokines and non-ELR-CXC chemokines (see e.g., Clark-Lewis, supra; and Belperio et al., “CXC Chemokines in Angiogenesis”, J. Leukoc. Biol. 68: 1-8, 2000).

Interferon-inducible protein-10 (IP-10 or CXCL10) (Accession No. NP_001556.2, P02778) is induced by interferon-gamma and TNF-alpha and produced by keratinocytes, endothelial cells, fibroblasts and monocytes. IP-10 is considered to play a role in mobilizing activated T cells to an inflammatory site of a tissue (Dufour, et al., “IFN-gamma-inducible protein 10 (IP-10; CXCL10)-deficient mice reveal a role for IP-10 in effector T cell generation and trafficking”, J Immunol., 168: 3195-204, 2002). Furthermore, there is a possibility that IP-10 plays a role in hypersensitive reaction. There is a possibility that IP-10 also plays a role in the occurrence of inflammatory demyelinating neuropathies (Kieseier, et al., “Chemokines and chemokine receptors in inflammatory demyelinating neuropathies: a central role for IP-10”, Brain 125: 823-34, 2002).

Research indicates the possibility that IP-10 is useful in the engraftment of stem cells following transplantation (Nagasawa, T., Int. J. Hematol. 72: 408-11, 2000), the mobilization of stem cells (Gazitt, Y., J. Hematother Stem Cell Res 10: 229-36, 2001; and Hattori et al., Blood 97: 3354-59, 2001) and antitumor hyperimmunity (Nomura et al., Int. J. Cancer 91: 597-606, 2001; and Mach and Dranoff, Curr. Opin. Immunol. 12: 571-75, 2000). For example, previous reports known to those skilled in the art discuss the biological activity of chemokine (Bruce, L. et al., “Radiolabeled Chemokine binding assays”, Methods in Molecular Biology (2000) vol. 138, pp. 129-134; Raphaele, B. et al., “Calcium Mobilization”, Methods in Molecular Biology (2000) vol. 138, pp. 143-148; and Paul D. Ponath et al., “Transwell Chemotaxis”, Methods in Molecular Biology (2000) vol. 138, pp. 113-120 Humana Press. Totowa, New Jersey).

Examples of the biological activity of CXCL10 include binding to a CXCL10 receptor (CXCR3), CXCL10-induced calcium flux, CXCL10-induced cell chemotaxis, binding of CXCL10 to glycosaminoglycan and CXCL10 oligomerization. Examples of the method for measuring the physiological activity of CXCL10 include a method of measuring the cell chemotactic activity of CXCL10, reporter assay using a cell line stably expressing CXCR3 (see PLoS One. 2010 Sep. 13; 5 (9): e12700), and PathHunter™ beta-Arrestin recruitment assay using B-arrestin recruitment induced at the early stage of GPCR signal transduction.

Programmed death 1 (PD-1) protein is an inhibitory member of the CD28 family of receptors. The CD28 family also includes CD28, CTLA-4, ICOS and BTLA. PD-1 is expressed on activated B cells, T cells and bone marrow cells (Okazaki et al., (2002) Curr. Opin. Immunol. 14: 391779-82; and Bennett et al., (2003) J Immunol 170: 711-8). CD28 and ICOS, the initial members of the family, were discovered on the basis of functional influence on the elevation of T cell growth after monoclonal antibody addition (Hutloff et al., (1999) Nature 397: 263-266; and Hansen et al., (1980) Immunogenics 10: 247-260). PD-1 was discovered by screening for differential expression in apoptotic cells (Ishida et al., (1992) EMBO J 11: 3887-95). CTLA-4 and BTLA, the other members of the family, were discovered by screening for differential expression in cytotoxic T lymphocytes and TH1 cells, respectively. CD28, ICOS and CTLA-4 all have an unpaired cysteine residue which permits homodimerization. In contrast, PD-1 is considered to exist as a monomer and lacks the unpaired cysteine residue characteristic of other members of the CD28 family.

The PD-1 gene encodes a 55 kDa type I transmembrane protein which is part of the Ig superfamily. PD-1 contains a membrane-proximal immunoreceptor tyrosine inhibitory motif (ITIM) and a membrane-distal tyrosine-based switch motif (ITSM). PD-1 is structurally similar to CTLA-4 but lacks a MYPPPY motif (SEQ ID NO: 159) important for B7-1 and B7-2 binding. Two ligands, PD-L1 and PD-L2, for PD-1 have been identified and have been shown to negatively regulate T-cell activation upon binding to PD-1 (Freeman et al., (2000) J Exp Med 192: 1027-34; Latchman et al., (2001) Nat Immunol 2: 261-8; and Carter et al., (2002) Eur J Immunol 32: 634-43). Both PD-L1 and PD-L2 are B7 homologs that bind to PD-1, but do not bind to the other members of the CD28 family. PD-L1, one of the PD-1 ligands, is abundant in various human cancers (Dong et al., (2002) Nat. Med. 8: 787-9). The interaction between PD-1 and PD-L1 results in decrease in tumor-infiltrating lymphocytes, reduction in T cell receptor-mediated growth, and immune evasion by the cancerous cells (Dong et al., (2003) J. Mol. Med. 81: 281-7; Blank et al., (2005) Cancer Immunol. Immunother. 54: 307-314; and Konishi et al., (2004) Clin. Cancer Res. 10: 5094-100). Immunosuppression can be reversed by inhibiting the local interaction of PD-1 with PD-Li, and this effect is additive when the interaction of PD-2 with PD-L2 is also inhibited (Iwai et al., (2002) Proc. Natl. Acad. Sci. USA 99: 12293-7; and Brown et al., (2003) J. Immunol. 170: 1257-66).

PD-1 is an inhibitory member of the CD28 family expressed on activated B cells, Tcells, and bone marrow cells. Animals deficient in PD-1 develop various autoimmune phenotypes, including autoimmune cardiomyopathy and lupus-like syndrome with arthritis and nephritis (Nishimura et al., (1999) Immunity 11: 141-51; and Nishimura et al., (2001) Science 291: 319-22). PD-1 has been further found to play an important role in autoimmune encephalomyelitis, systemic lupus erythematosus, graft-versus-host disease (GVHD), type I diabetes mellitus, and rheumatoid arthritis (Salama et al., (2003) J Exp Med 198: 71-78; Prokunia and Alarcon-Riquelme (2004) Hum Mol Genet 13: R143; and Nielsen et al., (2004) Lupus 13: 510). In a mouse B cell tumor line, the ITSM of PD-1 has been shown to be essential for inhibiting BCR-mediated Ca2+ flux and tyrosine phosphorylation of downstream effector molecules (Okazaki et al., (2001) PNAS 98: 13866-71).

In some embodiments, the ligand may be a wild-type (or naturally occurring) ligand or a mutant ligand with any mutation(s). In the case of IL-12, which is a heterodimeric cytokine, in some embodiments, the ligand, IL-12, may be wild-type (or naturally occurring) IL-12 or a mutant IL-12 with any mutation(s). In some embodiments, IL-12 may be a single-chain IL-12 in which p35 and p40 are linked to be contained in a single chain. In a particular embodiment, the ligand moiety/molecule of the present invention is IL-12 and the IL-12 is connected with C-terminal amino acid residue of the ligand-binding moiety/molecule via a peptide linker attached to p35 subunit of IL-12 or p40 subunit of IL-12. In one embodiment, the ligand moiety/molecule of the present invention is IL-12 and the IL-12 is connected with C-terminal amino acid residue of the ligand-binding moiety/molecule via a peptide linker attached to the N-terminus of p35 subunit of IL-12 or p40 subunit of IL-12. The same applies to any ligand in general or as described herein, including IL-22, etc.

In some embodiments of the present invention, a cytokine variant, a chemokine variant, or the like (e.g., Annu Rev Immunol. 2015; 33: 139-67) or a fusion protein containing the variants (e.g., Stem Cells Transl Med. 2015 January; 4 (1): 66-73) can be used as the ligand.

In some embodiments of the present invention, the ligand is selected from CXCL9, CXCL10, CXCL11, PD-1, IL-2, IL-12, IL-22, IL-6R, IL-1R1, IL-1R2, IL-1RAcP, and IL-1Ra. The CXCL10, PD-1, IL-2, IL-12, IL-22, IL-6R, IL-1R1, IL-1R2, IL-1RAcP, and IL-1Ra may have the same sequences as those of naturally occurring CXCL10, PD-1, IL-2, IL-12, IL-22, IL-6R, IL-1R1, IL-1R2, IL-1RAcP, and IL-1Ra, respectively, or may be a ligand variant that differs in sequence from naturally occurring CXCL9, CXCL10, CXCL11, PD-1, IL-2, IL-12, IL-22, IL-6R, IL-1R1, IL-1R2, IL-1RAcP, and IL-1Ra, but retains the physiological activity of the corresponding natural ligand. In order to obtain the ligand variant, an alteration may be artificially added to the ligand sequence for various purposes. Preferably, an alteration to resist protease cleavage (protease resistance alteration) is added thereto to obtain a ligand variant.

In some embodiments, the ligand binding moiety or molecule and the ligand moiety or molecule are fused via a peptide linker. In some embodiments, the VH or VL of the antigen-binding domain or variable region and the ligand moiety or molecule are fused via a peptide linker. For example, an arbitrary peptide linker that can be introduced by genetic engineering, or a linker disclosed as a synthetic compound linker (see e.g., Protein Engineering, 9 (3), 299-305, 1996) can be used as the linker in the fusion of the ligand binding molecule with the ligand. The length of the peptide linker is not particularly limited and may be appropriately selected by those skilled in the art according to the purpose. Examples of the peptide linker can include, but are not limited to:

Ser
Gly Ser (GS)
Ser Gly (SG)
Gly Gly Ser (GGS)
Gly Ser Gly (GSG)
Ser Gly Gly (SGG)
Gly Ser Ser (GSS)
Ser Ser Gly (SSG)
Ser Gly Ser (SGS)
Gly Gly Gly Ser (GGGS, SEQ ID NO: 136)
Gly Gly Ser Gly (GGSG, SEQ ID NO: 137)
Gly Ser Gly Gly (GSGG, SEQ ID NO: 138)
Ser Gly Gly Gly (SGGG, SEQ ID NO: 139)
Gly Ser Ser Gly (GSSG, SEQ ID NO: 140)
Gly Gly Gly Gly Ser (GGGGS, SEQ ID NO: 141)
Gly Gly Gly Ser Gly (GGGSG, SEQ ID NO: 142)
Gly Gly Ser Gly Gly (GGSGG, SEQ ID NO: 143)
Gly Ser Gly Gly Gly (GSGGG, SEQ ID NO: 144)
Gly Ser Gly Gly Ser (GSGGS, SEQ ID NO: 145)
Ser Gly Gly Gly Gly (SGGGG, SEQ ID NO: 146)
Gly Ser Ser Gly Gly (GSSGG, SEQ ID NO: 147)
Gly Ser Gly Ser Gly (GSGSG, SEQ ID NO: 148)
Ser Gly Gly Ser Gly (SGGSG, SEQ ID NO: 149)
Gly Ser Ser Ser Gly (GSSSG, SEQ ID NO: 150)
Gly Gly Gly Gly Gly Ser (GGGGGS, SEQ ID NO: 151)
Ser Gly Gly Gly Gly Gly (SGGGGG, SEQ ID NO: 152)
Gly Gly Gly Gly Gly Gly Ser (GGGGGGS, SEQ ID NO:
153)
Ser Gly Gly Gly Gly Gly Gly (SGGGGGG, SEQ ID NO:
154)
(Gly Gly Gly Gly Ser (GGGGS, SEQ ID NO: 141))n
(Ser Gly Gly Gly Gly (SGGGG, SEQ ID NO: 146))n
(Gly Gly Ser Gly Gly (GGSGG, SEQ ID NO: 143))n

wherein n is an integer of 1 or larger.

However, the length and sequence of the peptide linker can be appropriately selected by those skilled in the art according to the purpose.

The synthetic compound linker (chemical cross-linking agent) is a cross-linking agent usually used in peptide cross-linking, for example, N-hydroxysuccinimide (NHS), disuccinimidyl suberate (DSS), bis(sulfosuccinimidyl) suberate (BS3), dithiobis(succinimidyl propionate) (DSP), dithiobis(sulfosuccinimidyl propionate) (DTSSP), ethylene glycol bis(succinimidyl succinate) (EGS), ethylene glycol bis(sulfosuccinimidyl succinate) (sulfo-EGS), disuccinimidyl tartrate (DST), disulfosuccinimidyl tartrate (sulfo-DST), bis[2-(succinimidoxycarbonyloxy)ethyl]sulfone (BSOCOES), or bis[2-(sulfosuccinimidoxycarbonyloxy)ethyl]sulfone (sulfo-BSOCOES).

These cross-linking agents are commercially available.

### Ligand-Binding Moiety/Molecule

In a further aspect of the invention, the ligand-binding moiety/molecule such as a fusion protein comprises an antibody or a fragment thereof having ligand neutralising activity that is capable of inhibiting the biological activity of the ligand moiety/molecule by exerting its neutralising activity. In some embodiments, the fusion polypeptide comprises an antibody or a fragment thereof capable of binding to a ligand moiety/molecule (i.e. anti-ligand antibody). The antibody or a fragment thereof according to any of the above embodiments is a monoclonal antibody, including a chimeric, humanized or human antibody. In one embodiment, the antibody is an antibody fragment, e.g., a Fv, Fab, Fab′, scFv, diabody, or F(ab′)2 fragment. In another embodiment, the antibody is a full-length antibody, e.g., an intact IgG1 antibody or other antibody class or isotype as defined herein. In one embodiment, the antibody or fragment thereof is an IgG antibody-like polypeptide having moieties substantially similar in structure to constant domains or constant regions as in an IgG antibody, and moieties substantially similar in structure to variable domains or variable regions as in the IgG antibody, and having conformation substantially similar to that of the IgG antibody.

In a further aspect, the ligand-binding moiety/molecule such as the fusion polypeptide or antibody or antibody-like polypeptide or antibody fragment thereof according to any of the above embodiments may incorporate any of the features, singly or in combination, as described in the sections below.

The disclosure herein, e.g., the following passages, may in part mention or focus on “antibody”. However, as long as the antibody(-like) functions or characteristics described herein such as the ligand/antigen-binding activities are possessed or retained, the disclosure is suitably applicable to any types or formats of ligand/antigen-binding moieties/molecules such as any fusion protein/polypeptide or antibody or antibody-like polypeptide or antibody fragment thereof, etc., according to any of the embodiments described herein.

### Antibody Affinity

In certain embodiments, an antibody provided herein has a dissociation constant (Kd) of 1 micro M or less, 100 nM or less, 10 nM or less, 1 nM or less, 0.1 nM or less, 0.01 nM or less, or 0.001 nM or less (e.g. 108 M or less, e.g. from 108 M to 10⁻¹³ M, e.g., from 10⁻⁹ M to 10⁻¹³ M).

In one embodiment, Kd is measured by a radiolabelled antigen binding assay (RIA). In one embodiment, an RIA is performed with the Fab version of an antibody of interest, e.g. anti-ligand antibody and its ligand. For example, solution binding affinity of Fabs for ligand is measured by equilibrating Fab with a minimal concentration of (125I)-labelled ligand in the presence of a titration series of unlabelled ligand, then capturing bound ligand with an anti-Fab antibody-coated plate (see, e.g., Chen et al., J. Mol. Biol. 293:865-881(1999)). To establish conditions for the assay, MICROTITER (registered trademark) multi-well plates (Thermo Scientific) are coated overnight with 5 micro g/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23 degrees C.). In a non-adsorbent plate (Nunc #269620), 100 pM or 26 pM [125I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20 (registered trademark)) in PBS. When the plates have dried, 150 micro 1/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using a BIACORE (registered trademark) surface plasmon resonance (SPR) assay. For example, an assay using a BIACORE (registered trademark)-2000 or a BIACORE (registered trademark)-3000 (BIAcore, Inc., Piscataway, NJ) is performed at 25 degrees C. with immobilized antigen CM5 chips at about 10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Ligand is diluted with 10 mM sodium acetate, pH 4.8, to 5 micro g/ml (about 0.2 micro M) before injection at a flow rate of 5 micro 1/minute to achieve approximately 10 response units (RU) of coupled protein. Following the injection of ligand, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25 degrees C. at a flow rate of approximately 25 micro 1/min. Association rates (kon) and dissociation rates (koff) are calculated using a simple one-to-one Langmuir binding model (BIACORE (registered trademark) Evaluation Software version 3.2) by simultaneously fitting the association and dissociation sensorgrams. The equilibrium dissociation constant (Kd) is calculated as the ratio koff/kon. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25 degrees C. of a 20 nM anti-ligand antibody (Fab form) in PBS, pH 7.2, in the presence of increasing concentrations of ligand as measured in a spectrometer, such as a stop-flow equipped spectrophotometer (Aviv Instruments) or a 8000-series SLM-AMINCO™ spectrophotometer (ThermoSpectronic) with a stirred cuvette. Accordingly, persons skilled in the art can carry out affinity measurements using other common methods of measuring affinity for other ligand-binding molecules, antigen-binding molecules or antibodies, towards various kind of ligands, ligand receptors and antigens.

### Antibody Fragments

In certain embodiments, an antibody provided herein is an antibody fragment. Antibody fragments include, but are not limited to, Fab, Fab′, Fab′-SH, F(ab′)2, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)2 fragments comprising salvage receptor binding epitope residues and having increased in vivo half-life, see U.S. Pat. No. 5,869,046.

Diabodies are antibody fragments with two antigen-binding sites that may be bivalent or bispecific. In the present specification, diabodies also refer to antibody fragments with two ligand-binding domains that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

### Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extra chromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB (registered trademark) technology; U.S. Pat. No. 7,041,870 describing K-M MOUSE (registered trademark) technology, and U.S. Patent Application Publication No. US 2007/0061900, describing VELOCIMOUSE (registered trademark) technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

### Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N J, 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N J, 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

### Multispecific Antibodies

In certain embodiments, an antibody provided herein is a multispecific antibody, e.g. a bispecific antibody. Multispecific antibodies are monoclonal antibodies that have binding specificities for at least two different sites. In certain embodiments, one of the binding specificities is for an antigen and the other is for any other antigen. In certain embodiments, bispecific antibodies may bind to two different epitopes of an antigen. Bispecific antibodies may also be used to localize cytotoxic agents to cells which express the antigen. Bispecific antibodies can be prepared as full-length antibodies or antibody fragments.

In some embodiments, one of the binding specificities of the bispecific antibodies is for one ligand and the other is for any other ligand. Further, the bispecific antibody may bind two different epitopes of a ligand. The bispecific antibodies may also be used to localised cytotoxic agents to cells that express the ligand receptor. The bispecific antibodies can be prepared as full-length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (scFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1). The antibody or fragment herein also includes a “Dual Acting Fab” or “DAF” comprising an antigen binding site that binds to antigen as well as another, different antigen (see, US 2008/0069820, for example). In the present specification, engineered antibodies include antibodies with three or more functional ligand-binding domains and antibodies or fragments thereof comprising a ligand-binding domain that may bind to a ligand, as well as another, different ligand.

### Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding or ligand-binding.
a) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 2 under the heading of “preferred substitutions”. More substantial changes are provided in Table 2 under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 2

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; Met; Ala; | Ile |
| | Phe | |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr: Phe | Tyr |
| Tyr (Y) | Trp; Phe; Thr; Ser | Phe |
| Val (V) | Ile; Leu; Met; Phe; Ala; | Leu |
| | Norleucine | |

Amino acids may be grouped according to common side-chain properties:
(1) hydrophobic: Norleucine, Met, Ala, Val, Len, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding or ligand binding may be specifically identified, e.g., using alanine scanning mutagenesis or modelling. CDR-H3 and CDR-L3 in particular are often targeted.

In certain embodiments, substitutions, insertions, or deletions may occur within one or more HVRs so long as such alterations do not substantially reduce the ability of the antibody to bind antigen or ligand. For example, conservative alterations (e.g., conservative substitutions as provided herein) that do not substantially reduce binding affinity may be made in HVRs. Such alterations may, for example, be outside of antigen or ligand contacting residues in the HVRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen or ligand is affected. Further substitutions may be introduced at the amino acid locations demonstrating functional sensitivity to the initial substitutions. Alternatively, or additionally, a crystal structure of an antigen-antibody complex may be analysed to identify contact points between the antibody and antigen. Also included in the present specification, a crystal structure of a ligand-ligand-binding domain complex may be analysed to identify contact points between the ligand-binding domain and the ligand. Such contact residues and neighbouring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion of an enzyme (e.g. for ADEPT) or a polypeptide which increases the plasma half-life of the antibody to the N- or C-terminus of the antibody.

### b) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about +/−3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### c) Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks Fc gamma R binding (hence likely lacking ADCC activity) but retains FcRn binding ability. The primary cells for mediating ADCC, NK cells, express Fe gamma RIII only, whereas monocytes express Fe gamma RI, Fe gamma RII and Fe gamma RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACT1™ non-radioactive cytotoxicity assay for flow cytometry (CellTechnology, Inc. Mountain View, CA; and CytoTox 96 (registered trademark) non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with increased or decreased binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either increased or decreased) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and increased binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which increase binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

### d) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

### Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, polypropylene glycol homopolymers, polypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### Recombinant Methods and Compositions

Antibodies may be produced using recombinant methods and compositions, e.g., as described in U.S. Pat. No. 4,816,567. In one embodiment, isolated nucleic acid encoding an anti-ligand antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp2/0 cell). In one embodiment, a method of making an anti-ligand antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-ligand antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N J, 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumour (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

### Immunoconjugates

The invention also provides immunoconjugates comprising an anti-ligand antibody herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolacca americana proteins (PAPI, PAPII, and PAP-S), Momordica charantia inhibitor, curcin, crotin, Saponaria officinalis inhibitor, gelonin, mitogellin, restrictocin, phenomycin, enomycin, and the tricothecenes.

In another embodiment, an immunoconjugate comprises an antibody as described herein conjugated to a radioactive atom to form a radioconjugate. A variety of radioactive isotopes are available for the production of radioconjugates. Examples include 211At, 1311, 1251, 90Y, 186Re, 188Re, 153Sm, 212Bi, 32P, 212Pb and radioactive isotopes of Lu. When the radioconjugate is used for detection, it may comprise a radioactive atom for scintigraphic studies, for example Tc-99m or 1231, or a spin label for nuclear magnetic resonance (NMR) imaging (also known as magnetic resonance imaging, MRI), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionuclide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulphide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

### Assays

In some embodiments, the fusion polypeptide comprises a ligand-binding moiety/molecule that comprises an antibody or fragment thereof, wherein the antibody is a full-length antibody or IgG antibody-like polypeptide that binds to a ligand, i.e. anti-ligand antibody. Such anti-ligand antibodies provided herein may be identified, screened for, or characterized for their physical/chemical properties and/or biological activities by various assays known in the art.

### Binding Assays and Other Assays

In one aspect, an antibody of the invention (i.e. anti-ligand antibody) is tested for its ligand binding activity, e.g., by known methods such as ELISA, Western blot, etc.

In another aspect, competition assays may be used to identify an antibody that competes with other ligand-binding molecules for binding to the ligand. In certain embodiments, such a competing antibody binds to the same epitope (e.g., a linear or a conformational epitope) that is bound by the ligand-binding molecules. Detailed exemplary methods for mapping an epitope to which an antibody binds are provided in Morris (1996) “Epitope Mapping Protocols,” in Methods in Molecular Biology vol. 66 (Humana Press, Totowa, NJ).

In an exemplary competition assay, immobilized ligand is incubated in a solution comprising a first labelled antibody that binds to ligand (e.g., anti-ligand antibody or ligand-binding molecules) and a second unlabelled antibody that is being tested for its ability to compete with the first antibody for binding to ligand. The second antibody may be present in a hybridoma supernatant. As a control, immobilized ligand is incubated in a solution comprising the first labelled antibody but not the second unlabelled antibody. After incubation under conditions permissive for binding of the first antibody to ligand, excess unbound antibody is removed, and the amount of label associated with immobilized ligand is measured. If the amount of label associated with immobilized ligand is substantially reduced in the test sample relative to the control sample, then that indicates that the second antibody is competing with the first antibody for binding to ligand. See Harlow and Lane (1988) Antibodies: A Laboratory Manual ch. 14 (Cold Spring Harbour Laboratory, Cold Spring Harbour, NY).

### Activity Assays

In one aspect, assays are provided for identifying anti-ligand antibodies thereof having biological activity. Biological activity may include, e.g., binding to ligand receptor and activating ligand signalling. Antibodies having such biological activity in vivo and/or in vitro are also provided.

In certain embodiments, an antibody of the invention is tested for such biological activity. In vitro ligand activation can be confirmed by conducting ligand luciferase assay(s). Briefly, cells expressing ligand receptors were cultured. Anti-ligand antibodies were then incubated under conditions permissive for binding of the antibodies to the ligand receptors expressed on the cell surface. As a control, ligand is incubated under the same conditions as the test anti-ligand antibodies for binding to ligand receptors expressed on the cell surface. Luciferase activity is then detected using an appropriate assay system such as the Bio-Glo luciferase assay system (Promega, G7940) according to manufacturer's instructions. Luminescence can be detected using GloMax (registered trademark) Explorer System (Promega #GM3500) according to manufacturer's instructions. If comparable levels of activity for the anti-ligand antibodies and ligands are detected, it is demonstrated that the anti-ligand antibodies are capable of binding to ligand receptors and activating ligand signalling thereof.

### Exemplary IL-12 Fusion Polypeptides

In some embodiments of the present invention, the ligand moiety comprises IL-12 and the ligand-binding moiety/molecule comprises an antibody or fragment thereof, wherein the antibody is a full-length antibody or IgG antibody-like polypeptide that binds to IL-12, i.e. anti-IL-12 antibody.

In some embodiments of the present application, the ligand moiety comprises IL-12, and the fusion protein comprises any combinations of heavy and light chains selected from the group consisting of (i) to (vi):
(i) a light chain comprising the amino acid sequence that is at least 70%, 75%, 80%, 85%, 90%, 91%, 92%, 93%, 94%, 95%, 96%, 97%, 98%, 99% or 100% identical to SEQ ID NO: 1009;
(ii) a light chain comprising the amino acid sequence that is at least 70%, 75%, 80%, 85%, 90%, 91%, 92%, 93%, 94%, 95%, 96%, 97%, 98%, 99% or 100% identical to SEQ ID NO: 1016;
(iii) a light chain comprising the amino acid sequence that is at least 70%, 75%, 80%, 85%, 90%, 91%, 92%, 93%, 94%, 95%, 96%, 97%, 98%, 99% or 100% identical to SEQ ID NO: 1017;
(iv) a heavy chain comprising the amino acid sequence that is at least 70%, 75%, 80%, 85%, 90%, 91%, 92%, 93%, 94%, 95%, 96%, 97%, 98%, 99% or 100% identical to SEQ ID NO: 1012;
(v) a heavy chain comprising the amino acid sequence that is at least 70%, 75%, 80%, 85%, 90%, 91%, 92%, 93%, 94%, 95%, 96%, 97%, 98%, 99% or 100% identical to SEQ ID NO: 1050; and
(vi) a heavy chain comprising the amino acid sequence that is at least 70%, 75%, 80%, 85%, 90%, 91%, 92%, 93%, 94%, 95%, 96%, 97%, 98%, 99% or 100% identical to SEQ ID NO: 1088.

In some embodiments of the present application, the fusion protein comprises IL-12 binding moiety comprising any one of the combinations of heavy-chain variable region (VH) and light-chain variable region (VL) selected from (i) to (iii) below: (i) a heavy chain variable domain (VH) comprising the amino acid sequence that is at least 70%, 75%, 80%, 85%, 90%, 91%, 92%, 93%, 94%, 95%, 96%, 97%, 98%, 99% or 100% identical to SEQ ID NO: 1084;
(ii) a light chain variable domain (VL) comprising the amino acid sequence that is at least 70%, 75%, 80%, 85%, 90%, 91%, 92%, 93%, 94%, 95%, 96%, 97%, 98%, 99% or 100% identical to SEQ ID NO: 1085; and
(iii) a light chain variable domain (VL) comprising the amino acid sequence that is at least 70%, 75%, 80%, 85%, 90%, 91%, 92%, 93%, 94%, 95%, 96%, 97%, 98%, 99% or 100% identical to SEQ ID NO: 1086.

In another aspect, a fusion protein comprising a ligand-binding domain that binds IL-12 comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:1084. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but the ligand binding domain of the fusion protein comprising that sequence retains the ability to bind to IL-12. In certain embodiments, a total of 1 to 10 amino acids have been substituted, inserted and/or deleted in SEQ ID NO:1084. In certain embodiments, substitutions, insertions, or deletions occur in regions outside the HVRs (i.e., in the FRs). Optionally, the fusion protein comprising ligand-binding domain that binds IL-12 comprises the VH sequence in SEQ ID NO:1084, including post-translational modifications of that sequence. Post-translational modifications include but are not limited to a modification of glutamine or glutamate in N-terminal of heavy chain or light chain to pyroglutamic acid by pyroglutamylation.

In another aspect, a fusion protein comprising a ligand-binding domain that binds IL-12 is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:1085 or 1086. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but the ligand-binding domain of the fusion protein comprising that sequence retains the ability to bind to IL-12. In certain embodiments, a total of 1 to 10 amino acids have been substituted, inserted and/or deleted in SEQ ID NO:1085 or 1086. In certain embodiments, the substitutions, insertions, or deletions occur in regions outside the HVRs (i.e., in the FRs). Optionally, the fusion protein comprising ligand-binding domain that binds IL-12 comprises the VL sequence in SEQ ID NO:1085 or 1086, including post-translational modifications of that sequence. Post-translational modifications include but are not limited to a modification of glutamine or glutamate in N-terminal of heavy chain or light chain to pyroglutamic acid by pyroglutamylation.

In another aspect, a fusion protein comprising ligand-binding domain that binds IL-12 is provided, wherein the ligand-binding domain that binds IL-12 comprises a VH as in any of the embodiments provided above, and a VL as in any of the embodiments provided above. In one embodiment, the fusion protein comprising ligand-binding domain that binds IL-12 comprises the VH and VL sequences in SEQ ID NO:1012 or 1050, and SEQ ID NO: 1009, 1016 or 1017 respectively including post-translational modifications of those sequences. In a preferred embodiment, the fusion protein comprising ligand-binding domain that binds IL-12 comprises the VH and VL sequences in SEQ ID NO:1012 and SEQ ID NO:1009, respectively, the VH and VL sequences in SEQ ID NO:1050 and SEQ ID NO:1016, respectively, the VH and VL sequences in SEQ ID NO:1050 and SEQ ID NO:1017, respectively, including post-translational modifications of those sequences. Post-translational modifications include but are not limited to a modification of glutamine or glutamate in N-terminal of heavy chain or light chain to pyroglutamic acid by pyroglutamylation.

### Exemplary IL-22 Fusion Polypeptides

In some embodiments of the present invention, the ligand moiety comprises IL-22 and the ligand-binding moiety/molecule comprises an antibody or fragment thereof, wherein the antibody is a full-length antibody or IgG antibody-like polypeptide that binds to IL-22, i.e. anti-IL-22 antibody.

In some embodiments of the present application, the ligand moiety comprises IL-22, and the fusion protein comprises any combinations of heavy and light chains selected from the group consisting of:
(i) a light chain comprising the amino acid sequence that is at least 70%, 75%, 80%, 85%, 90%, 91%, 92%, 93%, 94%, 95%, 96%, 97%, 98%, 99% or 100% identical to SEQ ID NO: 1095;
(ii) a light chain comprising the amino acid sequence that is at least 70%, 75%, 80%, 85%, 90%, 91%, 92%, 93%, 94%, 95%, 96%, 97%, 98%, 99% or 100% identical to SEQ ID NO: 1097; and
(iii) a light chain comprising the amino acid sequence that is at least 70%, 75%, 80%, 85%, 90%, 91%, 92%, 93%, 94%, 95%, 96%, 97%, 98%, 99% or 100% identical to SEQ ID NO: 1099.

In some embodiments of the present application, the fusion protein comprises IL-22 binding moiety comprising any one of the combinations of heavy-chain variable region (VH) and light-chain variable region (VL) selected from:
(i) a heavy chain variable domain (VH) comprising the amino acid sequence that is at least 70%, 75%, 80%, 85%, 90%, 91%, 92%, 93%, 94%, 95%, 96%, 97%, 98%, 99% or 100% identical to SEQ ID NO: 1091;
(ii) a light chain variable domain (VL) comprising the amino acid sequence that is at least 70%, 75%, 80%, 85%, 90%, 91%, 92%, 93%, 94%, 95%, 96%, 97%, 98%, 99% or 100% identical to SEQ ID NO: 1092; and
(iii) a heavy chain variable domain (VH) comprising the amino acid sequence that is at least 70%, 75%, 80%, 85%, 90%, 91%, 92%, 93%, 94%, 95%, 96%, 97%, 98%, 99% or 100% identical to SEQ ID NO: 1093;
(iv) a light chain variable domain (VL) comprising the amino acid sequence that is at least 70%, 75%, 80%, 85%, 90%, 91%, 92%, 93%, 94%, 95%, 96%, 97%, 98%, 99% or 100% identical to SEQ ID NO: 1094.

In another aspect, a fusion protein comprising a ligand-binding domain that binds IL-22 comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 1091 or 1093. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but the ligand binding domain of the fusion protein comprising that sequence retains the ability to bind to IL-22. In certain embodiments, a total of 1 to 10 amino acids have been substituted, inserted and/or deleted in SEQ ID NO: 1091 or 1093. In certain embodiments, substitutions, insertions, or deletions occur in regions outside the HVRs (i.e., in the FRs). Optionally, the fusion protein comprising ligand-binding domain that binds IL-22 comprises the VH sequence in SEQ ID NO: 1091 or 1093, including post-translational modifications of that sequence. Post-translational modifications include but are not limited to a modification of glutamine or glutamate in N-terminal of heavy chain or light chain to pyroglutamic acid by pyroglutamylation.

In another aspect, a fusion protein comprising a ligand-binding domain that binds IL-22 is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 1092 or 1094. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but the ligand-binding domain of the fusion protein comprising that sequence retains the ability to bind to IL-22. In certain embodiments, a total of 1 to 10 amino acids have been substituted, inserted and/or deleted in SEQ ID NO: 1092 or 1094. In certain embodiments, the substitutions, insertions, or deletions occur in regions outside the HVRs (i.e., in the FRs). Optionally, the fusion protein comprising ligand-binding domain that binds IL-22 comprises the VL sequence in SEQ ID NO: 1092 or 1094, including post-translational modifications of that sequence. Post-translational modifications include but are not limited to a modification of glutamine or glutamate in N-terminal of heavy chain or light chain to pyroglutamic acid by pyroglutamylation.

In another aspect, a fusion protein comprising ligand-binding domain that binds IL-22 is provided, wherein the ligand-binding domain that binds IL-22 comprises a VH as in any of the embodiments provided above, and a VL as in any of the embodiments provided above. In one embodiment, the fusion protein comprising ligand-binding domain that binds IL-22 comprises the VH and VL sequences in SEQ ID NO: 1091 or 1093, and SEQ ID NO: 1092 or 1094 respectively including post-translational modifications of those sequences. In a preferred embodiment, the fusion protein comprising ligand-binding domain that binds IL-22 comprises the VH and VL sequences in SEQ ID NO: 1091 and SEQ ID NO: 1092, respectively, the VH and VL sequences in SEQ ID NO: 1093 and SEQ ID NO: 1094, respectively, including post-translational modifications of those sequences. Post-translational modifications include but are not limited to a modification of glutamine or glutamate in N-terminal of heavy chain or light chain to pyroglutamic acid by pyroglutamylation.

In a particular embodiment, the invention provides a fusion protein comprising a ligand that is interleukin-12 (IL-12), wherein the IL-12 has been modified to prevent its proteolytic degradation when exposed to a protease, i.e. protease-resistant IL-12. Modifications include amino acid modifications introduced to the IL-12 that prevent proteolytic degradation in the presence of protease, particularly wherein the modification is performed at the heparin binding site, which is in close proximity to the epitope of the variable region of the IL-12 fusion protein.

IL-12 comprises a heparin binding site which may be cleaved by a protease such as Human Matriptase/ST14 Catalytic Domain (MT-SP1). In a particular case of an IL-12 fusion protein, the heparin binding site may be in close proximity to the epitope of the variable region of the IL-12 fusion protein. Protease cleavage at the heparin binding site may affect the clearance of activated IL-12 fusion protein. Thus, in some embodiments, to prevent unintentional cleavage of IL-12 at the heparin binding site but preserve the ability of the epitope of the IL-12 fusion protein, at least one amino acid modifications may be introduced into the heparin binding site of IL-12. The term “protease resistant” or “protease resistance” as used herein, refers to the ability of a molecule comprising peptide bonds, such as a peptide, polypeptide or protein, to prevent hydrolytic cleavage of one or more of its peptide bonds in the presence of a protease. The degree of protease resistance may be measured by comparison with another molecule of the same identity that is less capable of withstanding hydrolytic cleavage when subjected in the presence of the same amount of protease under the same conditions at which the hydrolytic cleavage is evaluated. Protease cleavage can be confirmed when cleaved fragments of lower molecular weight than the original uncleaved parent molecule is obtained. Any methods known to the skilled person that are capable of detection of low molecular weight fragments derived from protease cleavage of a parent molecule may be used to evaluate protease resistance. Examples include subjecting protease resistant test variants and less protease resistant control to the same concentration of protease under the same conditions, including pH, temperature and duration. Subsequently, presence or absence of fragments of lower molecular weight derived from proteolytic cleavage may be observed by subjecting the treated test and control samples to reducing SDS-PAGE.

In a preferred embodiment, recombinant human matriptase/ST14 catalytic domain (MT-SP1) (R&D Systems, Inc., 3926-SE-010) was used as the protease. 75 nM protease and 750 nM of a fusion protein comprising protease resistance IL-12 were incubated in PBS under a condition of 37 degrees Celsius (degrees C.) for 1, 4 and 24 hours. Subsequently, presence of fragments of lower molecular weight than the parent after protease incubation, i.e. protease cleavage, was evaluated by reducing SDS-PAGE. A protease resistance variant is selected when the digested molecule remains more than 50%, 55%, 60%, 65%, 70%, 75%, 80%, 85%, 90%, 95%, 96%, 97%, 98%, 99% or 100% intact after subjected to proteolytic digestion of at least 4 hours, up to 24 hours under conditions stated above. Evaluation of percentage of a molecule remaining intact may be assessed in methods by various molecular biology techniques, including for example, SDS-PAGE densitometry of the lower molecular weight fragments (or none) derived from the parent molecule after digestion compared to the original parent molecule.

In some embodiments, the amino acid modification(s) are introduced to the heparin binding region of IL-12 which is prone to cleavage by proteases, including MT-SP1. Cleavage can occur in the heparin binding region between K260 and R261 of p40 subunit of the IL-12. In further embodiments, the amino acid modification(s) include(s) modification(s) of at least one, two, three, four, five or six positions of IL-12. Modification(s) or combination of modification(s) included herein, are not limited to the following list, as long as the IL-12 variant comprising said modification(s) or combination of modification(s) retains binding ability with IL-12 binding partners, e.g. IL-12Rb1, IL-12Rb2 and STAT4 to activate or initiate IL-12 signalling. In a preferred embodiment, after the modification(s) is/are performed on IL-12, the IL-12 (e.g., the heparin binding region of IL-12) may comprise a modified sequence selected from the following groups (a) to (p):

| | (a) |
|---|---|
| (SEQ ID NO: 1052) | |
| | KSHRE; |
| | (b) |
| (SEQ ID NO: 1053) | |
| | KSHHE; |
| | (c) |
| (SEQ ID NO: 1054) | |
| | KSHKE; |
| | (d) |
| (SEQ ID NO: 1055) | |
| | KSHSE; |
| | (e) |
| (SEQ ID NO: 1056) | |
| | KSKHRE; |
| | (f) |
| (SEQ ID NO: 1057) | |
| | KSKQRE; |
| | (g) |
| (SEQ ID NO: 1058) | |
| | KSKERE; |
| | (h) |
| (SEQ ID NO: 1059) | |
| | KSKPRE; |
| | (i) |
| (SEQ ID NO: 1060) | |
| | KHKE; |
| | (j) |
| (SEQ ID NO: 1061) | |
| | KHHE; |
| | (k) |
| (SEQ ID NO: 1062) | |
| | KHRE; |
| | (1) |
| (SEQ ID NO: 1063) | |
| | KKHE; |
| | (m) |
| (SEQ ID NO: 1064) | |
| | KRHE; |
| | (n) |
| (SEQ ID NO: 1065) | |
| | KRE; |
| | (o) |
| (SEQ ID NO: 1066) | |
| | KHE; |
| | and |
| | (p) |
| (SEQ ID NO: 1067) | |
| | KKE. |

In some embodiments of the present application, the protease resistant IL-12 comprises amino acid sequences selected from the group consisting of an amino acid sequence that is at least 70%, 75%, 80%, 85%, 90%, 91%, 92%, 93%, 94%, 95%, 96%, 97%, 98%, 99% or 100% identical to SEQ ID NO: 1068 to SEQ ID NO: 1083. In some embodiments, the protease resistant IL-12 comprises amino acid sequences selected from the group consisting of an amino acid sequence that is at least 70%, 80%, 90% or 100% identical to SEQ ID NO: 1068 to SEQ ID NO: 1083. In a preferred embodiment, the protease resistant IL-12 comprises amino acid sequences selected from the group consisting of an amino acid sequence that is at least 70%, 80%, 90% or 100% identical to SEQ ID NO: 1068, SEQ ID NO: 1069, or SEQ ID NO: 1076, or SEQ ID NO: 1077, or SEQ ID NO: 1078, or SEQ ID NO: 1079, or SEQ ID NO: 1080. In yet another preferred embodiment, the protease resistant IL-12 comprises amino acid sequences selected from the group consisting of an amino acid sequence that is identical to SEQ ID NO: 1068, SEQ ID NO: 1069, or SEQ ID NO: 1076, or SEQ ID NO: 1077, or SEQ ID NO: 1078, or SEQ ID NO: 1079, or SEQ ID NO: 1080.

In some embodiments, the protease resistant IL-12 does not comprise the amino acid sequence of KSKREK (SEQ ID NO: 1102) which is the native protease cleavage sequence.

In some embodiments, the protease resistant IL-12 (i) comprises an amino acid sequence that is at least 70%, 75%, 80%, 85%, 90%, 91%, 92%, 93%, 94%, 95%, 96%, 97%, 98%, 99% or 100% identical to an amino acid sequence selected from the group consisting of SEQ ID NO: 1068 to SEQ ID NO: 1083, and (ii) comprises an amino acid sequence selected from the group consisting of SEQ ID NO: 1052 to SEQ ID NO: 1067.

In some embodiments, the protease resistant IL-12 (i) comprises an amino acid sequence that is at least 70%, 75%, 80%, 85%, 90%, 91%, 92%, 93%, 94%, 95%, 96%, 97%, 98%, 99% or 100% identical to an amino acid sequence selected from the group consisting of SEQ ID NO: 1068 to SEQ ID NO: 1083, and (ii) does not comprise the amino acid sequence of KSKREK (SEQ ID NO: 1102).

In an aspect is included herein, a library comprising a fusion protein of any of the preceding embodiments, wherein the library is obtained by a method of screening for fusion proteins comprising one or more amino acid modifications that reduce association between VH and VL in the presence of a protease compared to in the absence of a protease, wherein the method of screening is as exemplified in any of the preceding embodiments.

In an aspect is included herein, a library comprising a fusion protein of any of the preceding embodiments, wherein the library is obtained by a method of producing fusion proteins comprising one or more amino acid modifications that reduce association between VH and VL in the presence of a protease compared to in the absence of a protease, wherein the one or more amino acid modifications that reduce association between VH and VL in the presence of a protease compared to in the absence of a protease is identified by a method of screening as exemplified in any of the preceding embodiments.

### III. Methods and Compositions for Diagnostics and Detection

In some embodiments, the fusion protein comprises a ligand-binding moiety/molecule that comprises an antibody or fragment thereof, wherein the antibody is a full-length antibody or IgG antibody-like polypeptide that binds to a ligand, i.e. anti-ligand antibody. As provided herein, the fusion protein or the anti-ligand antibody is useful for detecting the presence of ligand binding partner, such as ligand receptor in a biological sample. The term “detecting” as used herein encompasses quantitative or qualitative detection. In certain embodiments, a biological sample comprises a cell or tissue, such as cancer cell or tissue expressing ligand receptor, or inflammatory cell or tissue expressing ligand receptor, or any ligand receptor-expressing cell or tissue.

In one embodiment, the fusion protein or anti-ligand antibody for use in a method of diagnosis or detection is provided. In a further aspect, a method of detecting the presence of ligand binding partner, such as ligand receptor in a biological sample is provided. In certain embodiments, the method comprises contacting the biological sample with the fusion protein or anti-ligand antibody as described herein under conditions permissive for binding of the fusion protein or anti-ligand antibody to ligand, and detecting whether a complex is formed between the fusion protein or anti-ligand antibody and ligand. Such method may be an in vitro or in vivo method. In one embodiment, the fusion protein or an anti-ligand antibody is used to select subjects eligible for therapy with an anti-ligand antibody, e.g. where ligand is a biomarker for selection of patients.

In certain embodiments, labelled anti-ligand antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes 32P, 14C, 1251, 3H, and 131I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, beta-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, those coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### IV. Pharmaceutical Compositions/Formulations

The pharmaceutical composition of the present invention can be formulated by use of a method known to those skilled in the art. For example, the pharmaceutical composition can be parenterally used in an injection form of a sterile solution or suspension with water or any of other pharmaceutically acceptable liquids. The pharmaceutical composition can be formulated, for example, by appropriately combining the polypeptide with a pharmacologically acceptable carrier or medium, specifically, sterile water or physiological saline, a plant oil, an emulsifier, a suspending agent, a surfactant, a stabilizer, a flavouring agent, an excipient, a vehicle, an antiseptic, a binder, etc. and mixing them into a unit dosage form required for generally accepted pharmaceutical practice. The amount of the active ingredient in these formulations is set so as to give an appropriate volume in a prescribed range.

A sterile composition for injection can be formulated according to usual pharmaceutical practice using a vehicle such as injectable distilled water. Examples of the injectable aqueous solution include isotonic solutions containing physiological saline, glucose, or other adjuvants (e.g., D-sorbitol, D-mannose, D-mannitol, and sodium chloride). The aqueous solution can be used in combination with an appropriate solubilizer, for example, an alcohol (ethanol, etc.), a polyalcohol (propylene glycol, polyethylene glycol, etc.), or a nonionic surfactant (Polysorbate 80™, HCO-50, etc.).

Examples of the oil solution include sesame oil and soybean oil. The oil solution can also be used in combination with benzyl benzoate and/or benzyl alcohol as a solubilizer. The oil solution can be supplemented with a buffer (e.g., a phosphate buffer solution and a sodium acetate buffer solution), a soothing agent (e.g., procaine hydrochloride), a stabilizer (e.g., benzyl alcohol and phenol), and an antioxidant. The prepared injection solution is usually filled into an appropriate ampule.

The pharmaceutical composition of the present invention is preferably administered through a parenteral route. For example, a composition having an injection, transnasal, transpulmonary, or percutaneous dosage form is administered. The pharmaceutical composition can be administered systemically or locally by, for example, intravenous injection, intramuscular injection, intraperitoneal injection, or subcutaneous injection.

The administration method can be appropriately selected according to the age and symptoms of a patient. The dose of the pharmaceutical composition containing the ligand binding molecule can be set to the range of, for example, 0.0001 mg to 1000 mg per kg body weight per dose. Alternatively, the dose of the pharmaceutical composition containing the polypeptide can be set to a dose of, for example, 0.001 to 100000 mg per patient. However, the present invention is not necessarily limited by these numerical values. Although the dose and the administration method vary depending on the body weight, age, symptoms, etc. of a patient, those skilled in the art can set an appropriate dose and administration method in consideration of these conditions.

Pharmaceutical compositions/formulations of the present invention as described herein are prepared by mixing such fusion protein or antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX (registered trademark), Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized formulations are described in U.S. Pat. No. 6,267,958. Aqueous formulations include those described in U.S. Pat. No. 6,171,586 and WO2006/044908, the latter formulations including a histidine-acetate buffer.

The composition/formulation herein may also contain more than one active ingredient as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the fusion protein or antibody, which matrices are in the form of shaped articles, e.g. films, or microcapsules.

The compositions/formulations to be used for in vivo administration are generally sterile. Sterility may be readily accomplished, e.g., by filtration through sterile filtration membranes.

### V. Therapeutic Methods and Compositions

The present invention also relates to a pharmaceutical composition comprising the fusion protein or antibody of the present invention and a pharmaceutically acceptable carrier. In certain embodiments, the pharmaceutical composition of the disclosure is a cell growth-suppressing agent. In certain embodiments, the pharmaceutical composition of the disclosure is a pharmaceutical composition used for treatment and/or prevention of cancers or malignancies.

In certain embodiments, the pharmaceutical composition of the disclosure is a pharmaceutical composition used for treatment and/or prevention of inflammatory diseases. In certain embodiments, the pharmaceutical composition of the disclosure is a pharmaceutical composition used for treatment and/or prevention of gut or liver inflammatory diseases. In certain embodiments, the pharmaceutical composition of the disclosure is a pharmaceutical composition used for treatment and/or prevention of inflammatory bowel disease, alcoholic fatty liver disease, or non-alcoholic fatty liver disease. In certain embodiments, the pharmaceutical composition of the disclosure is a pharmaceutical composition used for treatment and/or prevention of Ulcerative Colitis or Crohn's Disease.

In certain embodiments, the pharmaceutical composition of the disclosure is a pharmaceutical composition used for treatment and/or prevention of autoimmune diseases. In certain embodiments, the pharmaceutical composition of the disclosure is a pharmaceutical composition used for treatment and/or prevention of rheumatoid arthritis, type 1 diabetes, and SLE.

The “treatment” (and its grammatically derived words, for example, “treat” and “treating”) used in the present specification means clinical intervention that intends to alter the natural course of an individual to be treated and can be carried out both for prevention and during the course of a clinical pathological condition. The desirable effect of the treatment includes, but is not limited to, the prevention of the development or recurrence of a disease, the alleviation of symptoms, the attenuation of any direct or indirect pathological influence of the disease, the prevention of metastasis, reduction in the rate of progression of the disease, recovery from or alleviation of a disease condition, and ameliorated or improved prognosis. In some embodiments, the ligand binding molecule of the present invention can control the biological activity of the ligand and is used for delaying the onset of a disease(s) or delaying the progression of the disease(s).

In the present invention, the pharmaceutical composition usually refers to a drug for the treatment or prevention of a disease or for examination or diagnosis.

In the present invention, the term “pharmaceutical composition comprising the fusion protein” may be used interchangeably with a “method for treating a disease, comprising administering the fusion protein to a subject to be treated” and may be used interchangeably with “use of the fusion protein for the production of a drug for the treatment of a disease”. Also, the term “pharmaceutical composition comprising the fusion protein” may be used interchangeably with “use of the fusion protein for treating a disease”.

In the present invention, the term “pharmaceutical composition comprising the antibody” may be used interchangeably with a “method for treating a disease, comprising administering the antibody to a subject to be treated” and may be used interchangeably with “use of the antibody for the production of a drug for the treatment of a disease”. Also, the term “pharmaceutical composition comprising the antibody” may be used interchangeably with “use of the antibody for treating a disease”.

In some embodiments of the present invention, the fusion protein or antibody of the present invention can be administered to an individual. The noncovalent bond still exists between the ligand-binding domain of the ligand-binding moiety/molecule and the ligand moiety.

In the case of administering the fusion protein of the present invention to an individual, the fusion protein is transported in vivo. The ligand-binding moiety in the fusion protein is cleaved in a target tissue so that the noncovalent bond of the ligand-binding domain of the ligand-binding molecule/moiety to the ligand is attenuated to release the ligand and a portion of the ligand-binding molecule from the fusion protein. The released ligand and the released portion of the ligand-binding molecule can exert the biological activity of the ligand in the target tissue and treat a disease caused by the target tissue. In the embodiments in which the ligand-binding moiety suppresses the biological activity of the ligand moiety when the ligand binding domain is bound with the ligand, and the ligand-binding moiety is cleaved specifically in a target tissue, the ligand in the fusion protein does not exert biological activity during transport and exerts biological activity only when the fusion protein is cleaved in the target tissue. As a result, the disease can be treated with less systemic adverse reactions.

In one aspect, the ligand moiety is IL-12 or IL-22 and the fusion protein or anti-ligand antibody binds IL-12 or IL-22. In this aspect, an IL-12 or IL-22 fusion protein or anti-IL-12 or anti-IL-22 antibody for use as a medicament is provided. In further aspects, an IL-12 or IL-22 fusion protein or anti-IL-12 or anti-IL-22 antibody for use in treating an IL-12 or IL-22 mediated disease is provided. In certain embodiments, an IL-12 or IL-22 fusion protein or anti-IL-12 or anti-IL-22 antibody for use in a method of treatment is provided. In certain embodiments, the invention provides an IL-12 or IL-22 fusion protein or anti-IL-12 or anti-IL-22 antibody for use in a method of treating an individual having an IL-12 or IL-22 mediated disease, or cancer, or malignancies, or inflammatory diseases, or autoimmune diseases, comprising administering to the individual an effective amount of the IL-12 or IL-22 fusion protein or anti-IL-12 or anti-IL-22 antibody. In one such embodiment, the method further comprises administering to the individual an effective amount of at least one additional therapeutic agent, e.g., as described below. In further embodiments, the invention provides an IL-12 or IL-22 fusion protein or anti-IL-12 or anti-IL-22 antibody for use in the modulation of IL-12 or IL-22 signalling in an individual. In certain embodiments, the invention provides an IL-12 or IL-22 fusion protein or an anti-IL-12 or anti-IL-22 antibody for use in a method of modulation of IL-12 or IL-22 signalling in an individual comprising administering to the individual an effective of the IL-12 or IL-22 fusion protein or anti-IL-12 or anti-IL-22 antibody to treat an IL-12 or IL-22 mediated disease, or cancer, or malignancies, or inflammatory diseases, or autoimmune diseases. An “individual” according to any of the above embodiments is preferably a human.

In a further aspect, the invention provides for the use of an IL-12 or IL-22 fusion protein or anti-IL-12 or anti-IL-22 antibody in the manufacture or preparation of a medicament. In one embodiment, the medicament is for treatment of an IL-12 or IL-22 mediated disease, or cancer, or malignancies, or inflammatory diseases, or autoimmune diseases. In a further embodiment, the medicament is for use in a method of treating an IL-12 or IL-22 mediated disease comprising administering to an individual having an IL-12 or IL-22 mediated disease, or cancer, or malignancies, or inflammatory diseases, or autoimmune diseases an effective amount of the medicament. In one such embodiment, the method further comprises administering to the individual an effective amount of at least one additional therapeutic agent, e.g., as described below. In a further embodiment, the medicament is for the modulation of IL-12 or IL-22 signalling in an individual. In a further embodiment, the medicament is for use in a method of the modulation of IL-12 or IL-22 signalling in an individual comprising administering to the individual an amount effective of the medicament to treat an IL-12 or IL-22 mediated disease, or cancer, or malignancies, or inflammatory diseases, or autoimmune diseases. An “individual” according to any of the above embodiments may be a human.

In a further aspect, the invention provides a method for treating an IL-12 or IL-22 mediated disease, or cancer, or malignancies, or inflammatory diseases, or autoimmune diseases. In one embodiment, the method comprises administering to an individual having such IL-12 or IL-22 mediated disease, or cancer, or malignancies, or inflammatory diseases, or autoimmune diseases an effective amount of an IL-12 or IL-22 fusion protein or anti-IL-12 or anti-IL-22 antibody. In one such embodiment, the method further comprises administering to the individual an effective amount of at least one additional therapeutic agent, as described below. An “individual” according to any of the above embodiments may be a human.

In a further aspect, the invention provides a method for modulation of IL-12 or IL-22 signalling in an individual. In one embodiment, the method comprises administering to the individual an effective amount of an IL-12 or IL-22 fusion protein or anti-IL-12 or anti-IL-22 antibody to modulate IL-12 or IL-22 signalling. In one embodiment, an “individual” is a human.

As aforementioned, treatment of an IL-12 or IL-22 mediated disease refers to the treatment of any disease, disorder, or condition susceptible of being improved or prevented by an increase or enhancement in IL-12 or IL-22 signalling. The present invention includes use of the fusion protein or anti-IL-12 or anti-IL-22 antibody described herein for use in the treatment of any disease, disorder, or condition susceptible of being improved or prevented by an increase or enhancement in IL-12 or IL-22 signalling. In a further aspect, the invention includes use of the fusion protein or anti-IL-12 or anti-IL-22 antibody described herein in the manufacture of a medicament for the treatment of any disease, disorder, or condition susceptible of being improved or prevented by an increase or enhancement in IL-12 or IL-22 signalling. In yet a further aspect, the invention includes the fusion protein or anti-IL-12 or anti-IL-22 antibody described herein in a method of treating any disease, disorder, or condition susceptible of being improved or prevented by an increase or enhancement in IL-12 or IL-22 signalling. In a further aspect, the modulation of IL-12 or IL-22 signalling is an increase or enhancement of IL-12 or IL-22 signalling that improves or prevents a disease, disorder or condition susceptible of being improved or prevented by said increase or enhancement in IL-12 or IL-22 signalling.

In one embodiment, the preferred cell types are IL-12 or IL-22 ligand receptor-expressing cells within the tumour microenvironment. In a preferred embodiment, the invention provides a method for treating a cancer or malignancy comprising administering to an individual having cancer, including but not limited to, for example, gastric cancer, head and neck cancer (H&N), esophageal cancer, lung cancer, liver cancer, ovary cancer, breast cancer, colon cancer, colorectal cancer, skin cancer, muscle tumor, pancreas cancer, prostate cancer, testis cancer, uterine cancer, cholangiocarcinoma, Merkel cell carcinoma, bladder cancer, thyroid cancer, schwannoma, adrenal cancer (adrenal gland), anus cancer, central nervous system tumor, neuroendocrine tissue tumor, penis cancer, pleura tumor, salivary gland tumor, vulva cancer, thymoma, and childhood cancer (Wilms tumor, neuroblastoma, sarcoma, hepatoblastoma, and germ cell tumor). Still more preferred cancer types include, but are not limited to, gastric cancer, head and neck cancer (H&N), esophageal cancer, lung cancer, liver cancer, ovary cancer, breast cancer, colon cancer, kidney cancer, skin cancer, muscle tumor, pancreas cancer, prostate cancer, testis cancer, and uterine cancer (Tumori. (2012) 98, 478-484; Tumor Biol. (2015) 36, 4671-4679; Am J Clin Pathol (2008) 130, 224-230; Adv Anat Pathol (2014) 21, 450-460; Med Oncol (2012) 29, 663-669; Clinical Cancer Research (2004) 10, 6612-6621; Appl Immunohistochem Mol Morphol (2009) 17, 40-46; Eur J Pediatr Surg (2015) 25, 138-144; J Clin Pathol (2011) 64, 587-591; Am J Surg Pathol (2006) 30, 1570-1575; Oncology (2007) 73, 389-394; Diagnostic Pathology (2010) 64, 1-6; Diagnostic Pathology (2015) 34, 1-6; Am J Clin Pathol (2008) 129, 899-906; Virchows Arch (2015) 466, 67-76).

In several embodiments, the individuals are patients who have received treatment with the above-described fusion protein or antibody or and/or some kind of anticancer agent(s) prior to the combination therapy using the fusion protein or antibody and an additional therapeutic agent. In several embodiments, patients are those who cannot receive standard therapy or for whom standard therapy is ineffective. In several embodiments, the cancer in which a patient has is early-stage or end-stage.

As used herein, “cancer” refers not only to epithelial malignancy such as ovary cancer or gastric cancer but also to non-epithelial malignancy including hematopoietic tumours such as chronic lymphocytic leukaemia or Hodgkin's lymphoma. Herein, the terms “cancer”, “carcinoma”, “tumour”, “neoplasm” and such are not differentiated from each other and are mutually interchangeable.

In a further aspect, the invention provides pharmaceutical compositions/formulations comprising any of the fusion protein or antibody provided herein, e.g., for use in any of the above therapeutic methods. In one embodiment, a pharmaceutical composition/formulation comprises any of the fusion protein or antibody provided herein and a pharmaceutically acceptable carrier. In another embodiment, a pharmaceutical composition/formulation comprises any of the fusion protein or antibody provided herein and at least one additional therapeutic agent, e.g., as described below.

Fusion proteins or antibodies of the present invention can be used either alone or in combination with other agents in a therapy. For instance, the fusion protein or antibodies of the present invention may be co-administered with at least one additional therapeutic agent. In certain embodiments, an additional therapeutic agent may be a cytostatic agent, a chemotherapeutic agent, or immunosuppressive agent. In one embodiment, an additional therapeutic agent is an immune checkpoint inhibitor.

Such combination therapies noted above encompass combined administration (where two or more therapeutic agents are included in the same or separate formulations), and separate administration, in which case, administration of the antibody of the invention can occur prior to, simultaneously, and/or following, administration of the additional therapeutic agent or agents. In one embodiment, administration of the fusion protein or antibody and administration of an additional therapeutic agent occur within about one month, or within about one, two or three weeks, or within about one, two, three, four, five, or six days, of each other. Fusion protein or antibody of the invention can also be used in combination with radiation therapy.

In a non-limiting embodiment of the present invention, pharmaceutical compositions (combination therapy) of the present invention can be used to treat patients who have cancer which is refractory to treatment with an immune checkpoint inhibitor. For example, patients with ligand-related cancer, in whom administration of an immune checkpoint inhibitor has failed to achieve a desired drug efficacy, can be treated with the pharmaceutical composition (combination therapy) of the present invention. In other words, ligand-related cancer that has been already treated with therapy using an immune checkpoint inhibitor can be treated with the pharmaceutical composition (combination therapy) of the present invention. Preferred examples of an additional therapeutic agent comprised in the pharmaceutical composition include immune checkpoint inhibitors but are not limited thereto.

In a non-limiting embodiment of the present invention, pharmaceutical compositions (combination therapy) of the present invention can be used to treat patients who have cancer which is refractory to treatment with the fusion protein or antibody of the present invention. For example, patients with ligand-related cancer, whose cancer has become resistant to the fusion protein or antibody of the present invention after administration of said proteins or antibodies or in whom administration of the proteins or antibodies of the present invention has failed to achieve a desired drug efficacy, can be treated with the pharmaceutical composition (combination therapy) of the present invention. In other words, ligand-related cancer that has been already treated with therapy using the fusion protein or antibody of the present invention can be treated with the pharmaceutical composition (combination therapy) of the present invention. Preferred examples of an additional therapeutic agent comprised in the pharmaceutical composition include immune checkpoint inhibitors but are not limited thereto.

A fusion protein or antibody of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Fusion proteins or antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The fusion protein or antibody need not be, but is optionally formulated with one or more agents currently used to prevent or treat the disorder in question. The effective amount of such other agents depends on the amount of fusion protein or antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of the fusion protein or antibody of the invention (when used alone or in combination with one or more other additional therapeutic agents) will depend on the type of disease to be treated, the type of antibody, the severity and course of the disease, whether the fusion protein or antibody is administered for preventive or therapeutic purposes, previous therapy, the patient's clinical history and response to the fusion protein or antibody, and the discretion of the attending physician. The fusion protein or antibody is suitably administered to the patient at one time or over a series of treatments. Depending on the type and severity of the disease, about 1 micro g/kg to 15 mg/kg (e.g. 0.1 mg/kg-10 mg/kg) of fusion protein or antibody can be an initial candidate dosage for administration to the patient, whether, for example, by one or more separate administrations, or by continuous infusion. One typical daily dosage might range from about 1 micro g/kg to 100 mg/kg or more, depending on the factors mentioned above. For repeated administrations over several days or longer, depending on the condition, the treatment would generally be sustained until a desired suppression of disease symptoms occurs. One exemplary dosage of the fusion protein or antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the fusion protein or antibody). An initial higher loading dose, followed by one or more lower doses may be administered. The progress of this therapy is easily monitored by conventional techniques and assays.

It is understood that any of the above formulations or therapeutic methods may be carried out using an immunoconjugate of the invention in place of or in addition to the fusion protein or antibody.

### VI. Articles of Manufacture

In another aspect of the invention, an article of manufacture containing materials useful for the treatment, prevention and/or diagnosis of the disorders described above is provided. The article of manufacture comprises a container and a label on or a package insert associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active ingredient in the composition is the fusion protein or antibody of the invention. The label or package insert indicates that the composition is used for treating the condition of choice. Moreover, the article of manufacture may comprise (a) a first container with a composition contained therein, wherein the composition comprises a fusion protein or an antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

It is understood that any of the above articles of manufacture may include an immunoconjugate of the invention in place of or in addition to the fusion protein or antibody.

### VII. Methods of Employing the Invention

The present invention also relates to a method for producing the fusion protein of the present invention. In one embodiment, the present invention provides a method for producing the fusion protein, comprising providing:
(a) a ligand-binding moiety comprising a ligand-binding domain and a protease cleavage site,
(b) a ligand molecule, and
(c) a non-cleavable peptide linker; and
connecting the ligand molecule to a C-terminal region of the ligand-binding molecule or an N-terminal region of the ligand-binding domain via the non-cleavable peptide linker.

In a preferable embodiment, the present invention provides a method for producing the bivalent fusion protein comprising two ligand-binding moieties, comprising providing for each ligand-binding moiety:
(a) a ligand-binding molecule comprising a ligand-binding domain and a protease cleavage site,
(b) a ligand molecule, and
(c) a non-cleavable peptide linker; and
connecting the ligand molecule to a C-terminal region of the ligand-binding molecule or an N-terminal region of the ligand-binding domain via the non-cleavable peptide linker in each ligand-binding moiety.

In one embodiment of the present invention, the method for producing a bivalent fusion protein comprising two ligand-binding moieties is a production method comprising the steps:
(a) obtaining an antibody comprising a variable region that binds to target ligand;
(b) introducing a protease cleavage sequence near the boundary of VH or VL and the constant region;
(c) introducing a first flexible linker in the hinge region between the variable region and Fc;
(d) introducing at least one amino acid mutation or at least one pair of amino acid mutation at the interface between VH and VL in said fusion protein or polypeptide, and optionally introducing at least one amino acid mutation at the interface between the ligand or antigen and the ligand-binding domain or antigen-binding domain, that promote dissociation of VH domain or VL domain from the fusion protein or polypeptide
(e) linking the molecule obtained in step (d) with the target ligand.

In one embodiment of the present invention, the method for producing a bivalent fusion protein further comprises:
(f) confirming that the ligand incorporated into the fusion protein has attenuated binding activity towards its binding partner when the fusion protein is in the uncleaved state.

In one embodiment of the present invention, the method for producing a bivalent fusion protein further comprises:
(g) confirming that the ligand incorporated into the fusion protein has restored binding activity towards its binding partner when the fusion protein is in the cleaved state.

In one embodiment of the present invention, the method for producing a bivalent fusion protein further comprises:
(h) confirming the amino acid modifications introduced in step (d) does not disrupt binding of ligand to the variable region of step (a)
(i) confirming the amino acid modifications introduced in step (d) reduces association between VH and VL in the second state than in the first state.

Examples of the method for introducing a protease cleavage sequence into a molecule capable of binding to a ligand include a method of inserting the protease cleavage sequence into the amino acid sequence of a polypeptide capable of binding to the ligand, and a method of replacing a portion of the amino acid sequence of a polypeptide capable of binding to the ligand with the protease cleavage sequence.

To “insert” amino acid sequence A into amino acid sequence B refers to splitting amino acid sequence B into two parts without deletion, and linking the two parts with amino acid sequence A (that is, producing such an amino acid sequence as “first half of amino acid sequence B—amino acid sequence A—second half of amino acid sequence B”). To “introduce” amino acid sequence A into amino acid sequence B refers to splitting amino acid sequence B into two parts and linking the two parts with amino acid sequence A. This encompasses not only “inserting” amino acid sequence A into amino acid sequence B as mentioned above, but also linking the two parts with amino acid sequence A after deleting one or more amino acid residues of amino acid sequence B including those adjacent to amino acid sequence A (that is, replacing a portion of amino acid sequence B with amino acid sequence A).

Examples of the method for obtaining the molecule capable of binding to a ligand include a method of obtaining a ligand-binding domain having the ability to bind to the ligand. The ligand-binding domain is obtained by a method using, for example, an antibody preparation method known in the art. The antibody obtained by the preparation method may be used directly in the fusion protein, or only a Fv region in the obtained antibody may be used. Where the Fv region in a single-chain (also referred to as “sc”) form can recognise the ligand, the single chain may be used. Alternatively, a Fab region containing the Fv region may be used.

A method for preparing an antibody or antibody fragment having desired binding activity is known to those skilled in the art. Hereinafter, a method for preparing an antibody binding to IL-12 or IL-22 (anti-IL-12 or anti-IL-22 antibody) will be given as an example. Antibodies binding to antigens other than IL-12 or IL-22 can also be appropriately prepared according to the example given below. It is within the expertise and knowledge of the skilled person to prepare an IgG antibody-like polypeptide and/or an antibody fragment(s) thereof having desired binding activity to IL-12 or IL-22 or antigens other than IL-12 or IL-22 by appropriately adapting the method described as example below accordingly.

The anti-IL-12 or anti-IL-22 antibody can be obtained as a polyclonal or monoclonal antibody by use of an approach known in the art. For example, monoclonal antibodies may be produced by a hybridoma method (Kohler and Milstein, Nature 256: 495 (1975)) or a recombination method (U.S. Pat. No. 4,816,567). Alternatively, the monoclonal antibodies may be isolated from phage displayed antibody libraries (Clackson et al., Nature 352: 624-628 (1991); and Marks et al., J. Mol. Biol. 222: 581-597 (1991)). Also, the monoclonal antibodies may be isolated from single B cell clones (N. Biotechnol. 28 (5): 253-457 (2011)).

A mammal-derived monoclonal antibody can be preferably prepared as the anti-IL-12 or anti-IL-22 antibody. The mammal-derived monoclonal antibody includes, for example, those produced by hybridomas and those produced by host cells transformed with an expression vector containing an antibody gene by a genetic engineering approach. The antibody described in the present application includes a “humanized antibody” and a “chimeric antibody”.

Humanized antibodies are also called reshaped human antibodies. Specifically, for example, a humanized antibody consisting of a non-human animal (e.g., mouse) antibody CDR-grafted human antibody is known in the art. General gene recombination approaches are also known for obtaining the humanized antibodies. Specifically, for example, overlap extension PCR is known in the art as a method for grafting mouse antibody CDRs to human FRs.

DNA encoding an antibody variable region containing three CDRs and four FRs linked and DNA encoding a human antibody constant region can be inserted into an expression vector such that these DNAs are fused in frame to prepare a vector for humanized antibody expression. The vector having the inserts is transfected into hosts to establish recombinant cells. Then, the recombinant cells are cultured for the expression of DNA encoding the humanized antibody to produce the humanized antibody into the cultures of the cultured cells (see European Patent Publication No. 239400 and International Publication No. WO1996/002576).

If necessary, FR amino acid residues may be substituted such that the CDRs of the reshaped human antibody form an appropriate antigen binding site. For example, a mutation can be introduced to the amino acid sequence of FR by the application of the PCR method used in the mouse CDR grafting to the human FRs.

The desired human antibody can be obtained by DNA immunization using transgenic animals having all repertoires of human antibody genes (see International Publication Nos. WO1993/012227, WO1992/003918, WO1994/002602, WO1994/025585, WO1996/034096, and WO1996/033735) as animals to be immunized.

In addition, a technique of obtaining human antibodies by panning using a human antibody library is also known. For example, a human antibody Fv region is expressed as a single-chain antibody (also referred to as “scFv”) on the surface of phages by a phage display method. A phage expressing antigen binding scFv can be selected. The gene of the selected phage can be analysed to determine a DNA sequence encoding the Fv region of the antigen binding human antibody. After the determination of the DNA sequence of the antigen binding scFv, the Fv region sequence can be fused in frame with the sequence of the desired human antibody C region and then inserted into an appropriate expression vector to prepare an expression vector. The expression vector is transfected into the preferred expression cells listed above for the expression of the gene encoding the human antibody to obtain the human antibody. These methods are already known in the art (see International Publication Nos. WO1992/001047, WO1992/020791, WO1993/006213, WO1993/011236, WO1993/019172, WO1995/001438, and WO1995/015388)

The monoclonal antibody-producing hybridomas can be prepared by use of a technique known in the art, for example, as follows: mammals are immunized with IL-12 or IL-22 protein used as a sensitizing antigen according to a usual immunization method. Immunocytes thus obtained are fused with parental cells known in the art by a usual cell fusion method. Next, cells producing a monoclonal antibody can be screened for by a usual screening method to select hybridomas producing the anti-IL-12 or anti-IL-22 antibody.

Specifically, the monoclonal antibody is prepared, for example, as follows: first, the IL-12 or IL-22 gene can be expressed to obtain the IL-12 or IL-22 protein which is used as a sensitizing antigen for antibody obtainment. Specifically, a gene sequence encoding IL-12 or IL-22 is inserted into an expression vector known in the art, with which appropriate host cells are then transformed. The desired human IL-12 or IL-22 protein is purified from the host cells or from a culture supernatant thereof by a method known in the art. In order to obtain soluble IL-12 or IL-22 from culture supernatant, for example, soluble IL-12 or IL-22 as described by Jayanthi et al. (Protein Expr Purif 2014 October; 102: 76-84) is expressed. Alternatively, purified natural IL-12 or IL-22 protein can also be used as a sensitizing antigen.

The purified IL-12 or IL-22 protein can be used as the sensitizing antigen for use in the immunization of mammals. A partial peptide of IL-12 or IL-22 can also be used as the sensitizing antigen. This partial peptide may be obtained by chemical synthesis from the amino acid sequence of human IL-12 or IL-22. Alternatively, the partial peptide may be obtained by the integration of a portion of the IL-12 or IL-22 gene to an expression vector followed by its expression. Furthermore, the partial peptide can also be obtained by the degradation of the IL-12 or IL-22 protein with a proteolytic enzyme. The region and size of the IL-12 or IL-22 peptide for use as such a partial peptide are not particularly limited by specific embodiments. The number of amino acids constituting the peptide as the sensitizing antigen is preferably at least 5 or more, for example, 6 or more or 7 or more. More specifically, a peptide of 8 to 50, preferably 10 to 30 residues can be used as the sensitizing antigen.

Also, a fusion protein of a desired partial polypeptide or peptide of the IL-12 or IL-22 protein fused with a different polypeptide can be used as the sensitizing antigen. For example, an antibody Fc fragment or a peptide tag can be preferably used for producing the fusion protein for use as the sensitizing antigen. A vector for the expression of the fusion protein can be prepared by fusing in frame genes encoding two or more types of the desired polypeptide fragments, and inserting the fusion gene into an expression vector as described above. The method for preparing the fusion protein is described in Molecular Cloning 2nd ed. (Sambrook, J. et al., Molecular Cloning 2nd ed., 9.47-9.58 (1989), Cold Spring Harbor Lab. Press). The method for obtaining IL-12 for use as the sensitizing antigen and the immunization method using this sensitizing antigen are also specifically described in WO2003/000883, WO2004/022754, WO2006/006693, etc.

The mammals to be immunized with the sensitizing antigen are not limited to particular animals. The mammals to be immunized are preferably selected in consideration of compatibility with the parental cells for use in cell fusion. In general, rodents (e.g., mice, rats, and hamsters), rabbits, monkeys, or the like are preferably used.

These animals are immunized with the sensitizing antigen according to a method known in the art. For example, a general immunization method involves administering the sensitizing antigen to the mammals by intraperitoneal or subcutaneous injection. Specifically, the sensitizing antigen diluted with PBS (phosphate-buffered saline), physiological saline, or the like at an appropriate dilution ratio is mixed, if desired, with a usual adjuvant, for example, a Freund's complete adjuvant and emulsified. Then, the resulting sensitizing antigen is administered to the mammals several times at 4- to 21-day intervals. Also, an appropriate carrier can be used in the immunization with the sensitizing antigen. Particularly, in the case of using a partial peptide having a small molecular weight as the sensitizing antigen, immunization with the sensitizing antigen peptide bound with a carrier protein such as albumin or keyhole limpet hemocyanin may be desirable in some cases.

Alternatively, the hybridomas producing the desired antibody can also be prepared as described below by use of DNA immunization. The DNA immunization is an immunization method which involves immunostimulating immunized animals by expressing in vivo the sensitizing antigen in the immunized animals given vector DNA that has been constructed in a form capable of expressing the gene encoding the antigenic protein in the immunized animals. The DNA immunization can be expected to be superior to the general immunization method using the administration of the protein antigen to animals to be immunized as follows:
the DNA immunization can provide immunostimulation with the structure of a membrane protein (e.g., IL-12 or IL-22) maintained; and
the DNA immunization eliminates the need of purifying the immunizing antigen.

In order to obtain the monoclonal antibody of the present invention by the DNA immunization, first, DNA for IL-12 or IL-22 protein expression is administered to animals to be immunized. The DNA encoding IL-12 or IL-22 can be synthesized by a method known in the art such as PCR. The obtained DNA is inserted into an appropriate expression vector, and then administered to the animals to be immunized. For example, a commercially available expression vector such as pcDNA3.1 can be preferably used as the expression vector. The vector can be administered to the organisms by a method generally used. For example, animal individuals are DNA-immunized by introducing into their cells gold particles with the expression vector adsorbed thereon using a gene gun. Furthermore, the antibody recognizing IL-12 or IL-22 can also be prepared by use of a method described in WO2003/104453.

A rise in the titer of the antibody binding to IL-12 or IL-22 is confirmed in the serum of the mammals thus immunized. Then, immunocytes are collected from the mammals and subjected to cell fusion. Particularly, spleen cells can be used as preferred immunocytes.

Mammalian myeloma cells are used in the cell fusion with the immunocytes. The myeloma cells preferably have an appropriate selection marker for screening. The selection marker refers to a trait that can survive (or cannot survive) under particular culture conditions. For example, hypoxanthine-guanine phosphoribosyltransferase deficiency (hereinafter, referred to as HGPRT deficiency) or thymidine kinase deficiency (hereinafter, referred to as TK deficiency) is known in the art as the selection marker. Cells having the HGPRT or TK deficiency are sensitive to hypoxanthineaminopterin-thymidine (hereinafter, referred to as HAT-sensitive). The HAT-sensitive cells are killed in a HAT selective medium because the cells fail to synthesize DNA. By contrast, these cells, when fused with normal cells, become able to grow even in the HAT selective medium because the fused cells can continue DNA synthesis through the use of the salvage pathway of the normal cells.

The cells having the HGPRT or TK deficiency can be selected in a medium containing 6-thioguanine or 8-azaguanine (hereinafter, abbreviated to 8AG) for the HGPRT deficiency or 5′-bromodeoxyuridine for the TK deficiency. The normal cells are killed by incorporating these pyrimidine analogs into their DNAs. By contrast, the cells deficient in these enzymes can survive in the selective medium because the cells cannot incorporate the pyrimidine analogs therein. In addition, a selection marker called G418 resistance confers resistance to a 2-deoxystreptamine antibiotic (gentamicin analog) through a neomycin resistance gene. Various myeloma cells suitable for cell fusion are known in the art.

For example, P3 (P3×63Ag8.653) (J. Immunol. (1979)123 (4), 1548-1550), P3×63Ag8U.1 (Current Topics in Microbiology and Immunology (1978)81, 1-7), NS-1 (C. Eur. J. Immunol. (1976)6 (7), 511-519), MPC-11 (Cell (1976)8 (3), 405-415), SP2/0 (Nature (1978)276 (5685), 269-270), FO (J. Immunol. Methods (1980)35 (1-2), 1-21), S194/5.XX0.BU.1 (J. Exp. Med. (1978)148 (1), 313-323), and R210 (Nature (1979)277 (5692), 131-133) can be preferably used as such myeloma cells.

Basically, the cell fusion of the immunocytes with the myeloma cells is performed according to a method known in the art, for example, the method of Kohler and Milstein et al. (Methods Enzymol. (1981) 73, 3-46). More specifically, the cell fusion can be carried out, for example, in a usual nutrient medium in the presence of a cell fusion promoter. For example, polyethylene glycol (PEG) or hemagglutinating virus of Japan (HVJ) is used as the fusion promoter. In addition, an auxiliary such as dimethyl sulfoxide is added thereto for use, if desired, for enhancing fusion efficiency.

The ratio between the immunocytes and the myeloma cells used can be arbitrarily set. For example, the amount of the immunocytes is preferably set to 1 to 10 times the amount of the myeloma cells. For example, an RPMI1640 medium or a MEM medium suitable for the growth of the myeloma cell line as well as a usual medium for use in this kind of cell culture is used as the medium in the cell fusion. Preferably, a solution supplemented with serum (e.g., fetal calf serum (FCS)) can be further added to the medium.

For the cell fusion, the immunocytes and the myeloma cells are well mixed in the predetermined amounts in the medium. A PEG solution (e.g., average molecular weight of PEG: on the order of 1000 to 6000) preheated to approximately 37 degrees Celsius (C) is usually added thereto at a concentration of 30 to 60% (w/v). The mixed solution is gently mixed so that the desired fusion cells (hybridomas) are formed. Subsequently, the appropriate medium listed above is sequentially added to the cell cultures, and its supernatant is removed by centrifugation. This operation can be repeated to remove the cell fusion agents or the like unfavourable for hybridoma growth.

The hybridomas thus obtained can be cultured in a usual selective medium, for example, a HAT medium (medium containing hypoxanthine, aminopterin, and thymidine), for selection. The culture using the HAT medium can be continued for a time long enough to kill cells (non-fused cells) other than the desired hybridomas (usually, the time long enough is several days to several weeks). Subsequently, the hybridomas producing the desired antibody are screened for and single-cell cloned by a usual limiting dilution method.

The hybridomas thus obtained can be selected through the use of a selective medium appropriate for the selection marker of the myeloma cells used in the cell fusion. For example, the cells having the HGPRT or TK deficiency can be selected by culture in a HAT medium (medium containing hypoxanthine, aminopterin, and thymidine). Specifically, in the case of using HAT-sensitive myeloma cells in the cell fusion, only cells successfully fused with normal cells can be grown selectively in the HAT medium. The culture using the HAT medium is continued for a time long enough to kill cells (non-fused cells) other than the desired hybridomas. Specifically, the culture can generally be performed for several days to several weeks to select the desired hybridomas. Subsequently, the hybridomas producing the desired antibody can be screened for and single-cell cloned by a usual limiting dilution method.

The screening of the desired antibody and the single-cell cloning can be preferably carried out by a screening method based on antigen-antibody reaction known in the art. For example, a monoclonal antibody binding to IL-12 or IL-22 can bind to IL-12 or IL-22 expressed on cell surface. Such a monoclonal antibody can be screened for by, for example, FACS (fluorescence activated cell sorting). FACS is a system capable of measuring the binding of an antibody to cell surface by analyzing cells contacted with a fluorescent antibody using laser beam, and measuring the fluorescence emitted from the individual cells.

In order to screen for hybridomas producing a monoclonal antibody of interest by FACS, first, IL-12- or IL-22-expressing cells are prepared. Cells preferred for screening are mammalian cells forced to express IL-12 or IL-22. Untransformed, host mammalian cells can be used as a control to selectively detect the binding activity of an antibody against IL-12 or IL-22 on cell surface. Specifically, hybridomas producing an antibody that does not bind to the control host cells but binds to the cells forced to express IL-12 or IL-22 are selected to obtain hybridomas producing a monoclonal antibody against IL-12 or IL-22.

Alternatively, the antibody can be evaluated for its binding activity against immobilized IL-12- or IL-22-expressing cells on the basis of the principle of ELISA. The IL-12- or IL-22-expressing cells are immobilized onto each well of, for example, an ELISA plate. The hybridoma culture supernatant is contacted with the immobilized cells in the well to detect an antibody binding to the immobilized cells. When the monoclonal antibody is derived from a mouse, the antibody bound with the cell can be detected using an anti-mouse immunoglobulin antibody. Hybridomas selected by these screening methods, which produce a desired antibody capable of binding to the antigen, can be cloned by limiting dilution method or other methods.

The monoclonal antibody-producing hybridomas thus prepared can be subcultured in a usual medium. The hybridomas can also be preserved over a long period in liquid nitrogen.

The hybridomas are cultured according to a usual method, and the desired monoclonal antibody can be obtained from the culture supernatant thereof. Alternatively, the hybridomas may be administered to mammals compatible therewith and grown, and the monoclonal antibody can be obtained from the ascitic fluids thereof.

The former method is suitable for obtaining highly pure antibodies.

An antibody encoded by an antibody gene cloned from the antibody-producing cells such as hybridomas can also be preferably used. The cloned antibody gene is integrated to an appropriate vector, which is then transfected into hosts so that the antibody encoded by the gene is expressed. Methods for the isolation of the antibody gene, the integration to a vector, and the transformation of host cells have already been established by, for example, Vandamme et al. (Eur. J. Biochem. (1990) 192 (3), 767-775). A method for producing a recombinant antibody as mentioned below is also known in the art.

For example, cDNA encoding the variable region (V region) of an anti-IL-12 or anti-IL-22 antibody is obtained from a hybridoma cell producing the anti-IL-12 or anti-IL-22 antibody. For this purpose, usually, total RNA is first extracted from the hybridoma. For example, mRNA can be extracted from the cell using any of the following methods:
a guanidine ultracentrifugation method (Biochemistry (1979) 18 (24), 5294-5299), and
an AGPC method (Anal. Biochem. (1987) 162 (1), 156-159).

The extracted mRNA can be purified using mRNA Purification Kit (manufactured by GE Healthcare Bio-Sciences Corp.) or the like. Alternatively, a kit for directly extracting total mRNA from cells is also commercially available, such as QuickPrep mRNA Purification Kit (manufactured by GE Healthcare Bio-Sciences Corp.). The mRNA can be obtained from the hybridomas using such a kit. From the obtained mRNA, the cDNA encoding the antibody V region can be synthesized using reverse transcriptase. The cDNA can be synthesized using, for example, AMV Reverse Transcriptase First-strand cDNA Synthesis Kit (manufactured by Seikagaku Corp.). Alternatively, a 5′-RACE method (Proc. Natl. Acad. Sci. USA (1988) 85 (23), 8998-9002; and Nucleic Acids Res. (1989) 17 (8), 2919-2932) using SMART RACE cDNA amplification kit (manufactured by Clontech Laboratories, Inc.) and PCR can be appropriately used for the cDNA synthesis and amplification. In the course of such cDNA synthesis, appropriate restriction sites mentioned later can be further introduced to both ends of the cDNA.

The cDNA fragment of interest is purified from the obtained PCR product and subsequently ligated with vector DNA. The recombinant vector thus prepared is transfected into E. coli or the like. After colony selection, the desired recombinant vector can be prepared from the E. coli that has formed the colony. Then, whether or not the recombinant vector has the nucleotide sequence of the cDNA of interest is confirmed by a method known in the art, for example, a dideoxynucleotide chain termination method.

The 5′-RACE method using primers for variable region gene amplification is conveniently used for obtaining the gene encoding the variable region. First, a 5′-RACE cDNA library is obtained by cDNA synthesis with RNAs extracted from the hybridoma cells as templates. A commercially available kit such as SMART RACE cDNA amplification kit is appropriately used in the synthesis of the 5′-RACE cDNA library.

The antibody gene is amplified by PCR with the obtained 5′-RACE cDNA library as a template. Primers for mouse antibody gene amplification can be designed on the basis of an antibody gene sequence known in the art. These primers have nucleotide sequences differing depending on immunoglobulin subclasses. Thus, the subclass is desirably determined in advance using a commercially available kit such as Iso Strip mouse monoclonal antibody isotyping kit (Roche Diagnostics K.K.).

Specifically, primers capable of amplifying genes encoding gamma 1, gamma 2a, gamma 2b, and gamma 3 heavy chains and kappa and lambda light chains can be used, for example, for the purpose of obtaining a gene encoding mouse IgG. In order to amplify an IgG variable region gene, a primer that anneals to a moiety corresponding to a constant region close to the variable region is generally used as a 3′ primer. On the other hand, a primer attached to the 5′ RACE cDNA library preparation kit is used as a 5′ primer.

The PCR products thus obtained by amplification can be used to reconstruct immunoglobulins composed of heavy and light chains in combination. The reconstructed immunoglobulins can be screened for binding activity to IL-12 or IL-22 to obtain a desired antibody. More preferably, the binding of the antibody to IL-12 or IL-22 is specific, for example, for the purpose of obtaining the antibody against IL-12 or IL-22. An antibody binding to IL-12 or IL-22 can be screened for, for example, by the following steps:
(1) contacting an antibody containing the V region encoded by the cDNA obtained from a hybridoma, with IL-12- or IL-22-expressing cells;
(2) detecting the binding of the antibody to the IL-12- or IL-22-expressing cells; and
(3) selecting the antibody binding to the IL-12- or IL-22-expressing cells.

A method for detecting the binding of the antibody to the IL-12- or IL-22-expressing cells is known in the art. Specifically, the binding of the antibody to the IL-12- or IL-22-expressing cells can be detected by an approach such as FACS mentioned above. A fixed preparation of IL-12- or IL-22-expressing cells can be appropriately used for evaluating the binding activity of the antibody.

A panning method using phage vectors is also preferably used as a method for screening for the antibody with binding activity as an index. When antibody genes are obtained as libraries of heavy chain and light chain subclasses from a polyclonal antibody-expressing cell population, a screening method using phage vectors is advantageous. Genes encoding heavy chain and light chain variable regions can be linked via an appropriate linker sequence to form a gene encoding single-chain Fv (scFv). The gene encoding scFv can be inserted into phage vectors to obtain phages expressing scFv on their surface. After contact of the phages with the desired antigen, phages bound with the antigen can be recovered to recover DNA encoding scFv having the binding activity of interest. This operation can be repeated, if necessary, to enrich scFvs having the desired binding activity.

After the obtainment of the cDNA encoding the V region of the anti-IL-12 or anti-IL-22 antibody of interest, this cDNA is digested with restriction enzymes that recognize the restriction sites inserted at both ends of the cDNA. The restriction enzymes preferably recognize and digest a nucleotide sequence that appears low frequently in the nucleotide sequence constituting the antibody gene. The insertion of sites for restriction enzymes that provide cohesive ends is preferred for inserting one copy of the digested fragment in the correct orientation into a vector. The thus-digested cDNA encoding the V region of the anti-IL-12 or anti-IL-22 antibody can be inserted into an appropriate expression vector to obtain an antibody expression vector. In this case, a gene encoding an antibody constant region (C region) and the gene encoding the V region are fused in frame to obtain a chimeric antibody. In this context, the “chimeric antibody” refers to an antibody having constant and variable regions of different origins. Thus, heterogeneous (e.g., mouse-human) chimeric antibodies as well as human-human homogeneous chimeric antibodies are also included in the chimeric antibody according to the present invention. The V region gene can be inserted into an expression vector preliminarily having a constant region gene to construct a chimeric antibody expression vector. Specifically, for example, recognition sequences for restriction enzymes digesting the V region gene can be appropriately placed on the 5′ side of an expression vector carrying the DNA encoding the desired antibody constant region (C region).

This expression vector having the C region gene and the V region gene are digested with the same combination of restriction enzymes and fused in frame to construct a chimeric antibody expression vector.

In order to produce the anti-IL-12 or anti-IL-22 monoclonal antibody, the antibody gene is integrated to an expression vector such that the antibody gene is expressed under the control of expression control regions. The expression control regions for antibody expression include, for example, an enhancer and a promoter. Also, an appropriate signal sequence can be added to the amino terminus such that the expressed antibody is extracellularly secreted. For example, a peptide having an amino acid sequence MGWSCIILFLVATATGVHS (SEQ ID NO: 1) can be used as the signal sequence. Any of other suitable signal sequences may be added thereto. The expressed polypeptide is cleaved at the carboxyl-terminal moiety of this sequence. The cleaved polypeptide can be extracellularly secreted as a mature polypeptide. Subsequently, appropriate host cells can be transformed with this expression vector to obtain recombinant cells expressing the DNA encoding the anti-IL-12 or anti-IL-22 antibody.

The molecule harbouring the protease cleavage sequence in the molecule capable of binding to a ligand serves as the ligand binding moiety or molecule in the present invention. Whether the ligand binding moiety/molecule is cleaved by treatment with protease appropriate for the protease cleavage sequence can be optionally confirmed. The presence or absence of the cleavage of the protease cleavage sequence can be confirmed, for example, by contacting the protease with the molecule harbouring the protease cleavage sequence in the molecule capable of binding to a ligand, and confirming the molecular weight of the protease treatment product by an electrophoresis method such as SDS-PAGE.

Furthermore, cleavage fragments after protease treatment can be separated by electrophoresis such as SDS-PAGE and quantified to evaluate the activity of the protease and the cleavage ratio of a molecule into which the protease cleavage sequence has been introduced. A non-limiting embodiment of the method of evaluating the cleavage ratio of a molecule into which a protease cleavage sequence has been introduced includes the following method: For example, when the cleavage ratio of an antibody variant into which a protease cleavage sequence has been introduced is evaluated using recombinant human u-Plasminogen Activator/Urokinase (human uPA, huPA) (R&D Systems; 1310-SE-010) or recombinant human Matriptase/ST14 Catalytic Domain (human MT-SP1, hMT-SP1) (R&D Systems; 3946-SE-010), 100 microgram/mL of the antibody variant is reacted with 40 nM huPA or 3 nM hMT-SP1 in PBS at 37 degrees C. for one hour, and then subjected to capillary electrophoresis immunoassay. Capillary electrophoresis immunoassay can be performed using Wes (Protein Simple), but the present method is not limited thereto. As an alternative to capillary electrophoresis immunoassay, SDS-PAGE and such may be performed for separation, followed by detection with Western blotting. The present method is not limited to these methods. Before and after cleavage, the light chain can be detected using anti-human lambda chain HRP-labelled antibody (abeam; ab9007), but any antibody that can detect cleavage fragments may be used. The area of each peak obtained after protease treatment is output using software for Wes (Compass for SW; Protein Simple), and the cleavage ratio (%) of the antibody variant can be determined with the following formula:

(Peak area of cleaved light chain)×100/(Peak area of cleaved light chain+Peak area of uncleaved light chain)

Cleavage ratios can be determined if protein fragments can be detected before and after protease treatment. Thus, cleavage ratios can be determined not only for antibody variants but also for various protein molecules into which a protease cleavage sequence has been introduced.

The in vivo cleavage ratio of a molecule into which a protease cleavage sequence has been introduced can be determined by administering the molecule into animals and detecting the administered molecule in blood samples. For example, an antibody variant into which a protease cleavage sequence has been introduced is administered to mice, and plasma is collected from their blood samples. The antibody is purified from the plasma by a method known to those skilled in the art using Dynabeads Protein A (Thermo; 10001D), and then subjected to capillary electrophoresis immunoassay to evaluate the protease cleavage ratio of the antibody variant. Capillary electrophoresis immunoassay can be performed using Wes (Protein Simple), but the present method is not limited thereto. As an alternative to capillary electrophoresis immunoassay, SDS-PAGE and such may be performed for separation, followed by detection with Western blotting. The present method is not limited to these methods. The light chain of the antibody variant collected from mice can be detected using anti-human lambda chain HRP-labelled antibody (abeam; ab9007), but any antibody that can detect cleavage fragments may be used. Once the area of each peak obtained by capillary electrophoresis immunoassay is output using software for Wes (Compass for SW; Protein Simple), the ratio of the remaining light chain can be calculated as [Peak area of light chain]/[Peak area of heavy chain] to determine the ratio of the full-length light chain that remain uncleaved in the mouse body. In vivo cleavage efficiencies can be determined if protein fragments collected from a living organism are detectable. Thus, cleavage ratios can be determined not only for antibody variants but also for various protein molecules into which a protease cleavage sequence has been introduced. Calculation of cleavage ratios by the above-mentioned methods enables, for example, comparison of the in vivo cleavage ratios of antibody variants into which different cleavage sequences have been introduced, and comparison of the cleavage ratio of a single antibody variant between different animal models such as a normal mouse model and a tumour-grafted mouse model.

The present invention also relates to a polynucleotide encoding the fusion protein of the present invention.

The polynucleotide according to the present invention is usually carried by (or inserted in) an appropriate vector and transfected into host cells. The vector is not particularly limited as long as the vector can stably retain an inserted nucleic acid. For example, when E. coli is used as the host, a pBluescript vector (manufactured by Stratagene Corp.) or the like is preferred as a vector for cloning. Various commercially available vectors can be used. In the case of using the vector for the purpose of producing the fusion protein of the present invention, an expression vector is particularly useful. The expression vector is not particularly limited as long as the vector permits expression of the fusion protein in vitro, in E. coli, in cultured cells, or in organism individuals. The expression vector is preferably, for example, a pBEST vector (manufactured by Promega Corp.) for in vitro expression, a pET vector (manufactured by Invitrogen Corp.) for E. coli, a pME18S-FL3 vector (GenBank Accession No. AB009864) for cultured cells, and a pME18S vector (Mol Cell Biol. 8:466-472 (1988)) for organism individuals. The insertion of the DNA of the present invention into the vector can be performed by a routine method, for example, ligase reaction using restriction sites (Current protocols in Molecular Biology edit. Ausubel et al. (1987) Publish. John Wiley & Sons. Section 11.4-11.11).

The host cells are not particularly limited, and various host cells are used according to the purpose. Examples of the cells for expressing the fusion protein can include bacterial cells (e.g., Streptococcus, Staphylococcus, E. coli, Streptomyces, and Bacillus subtilis), fungal cells (e.g., yeasts and Aspergillus), insect cells (e.g., Drosophila S2 and Spodoptera SF9), animal cells (e.g., CHO, COS, HeLa, C127, 3T3, BHK, HEK293, and Bowes melanoma cells) and plant cells. The transfection of the vector to the host cells may be performed by a method known in the art, for example, a calcium phosphate precipitation method, an electroporation method (Current protocols in Molecular Biology edit. Ausubel et al., (1987) Publish. John Wiley & Sons. Section 9.1-9.9), a Lipofectamine method (manufactured by GIBCO-BRL/Thermo Fisher Scientific Inc.), or a microinjection method.

An appropriate secretory signal can be incorporated into the fusion protein of interest in order to secrete the fusion protein expressed in the host cells to the lumen of the endoplasmic reticulum, periplasmic space, or an extracellular environment. The signal may be endogenous to the fusion protein of interest or may be a foreign signal.

When the fusion protein of the present invention is secreted into a medium, the recovery of the fusion protein in the production method is performed by the recovery of the medium. When the fusion protein of the present invention is produced into cells, the cells are first lysed, followed by the recovery of the fusion protein.

A method known in the art including ammonium sulphate or ethanol precipitation, acid extraction, anion- or cation-exchange chromatography, phosphocellulose chromatography, hydrophobic interaction chromatography, affinity chromatography, hydroxyapatite chromatography, and lectin chromatography can be used for recovering and purifying the fusion protein of the present invention from the recombinant cell cultures.

It should be understood by those skilled in the art that arbitrary combinations of one or more embodiments described in the present specification are also included in the present invention unless there is technical contradiction on the basis of the technical common sense of those skilled in the art. Also, the present invention excluding arbitrary combinations of one or more embodiments described in the present specification is intended in the present specification and should be interpreted as the described invention, unless there is technical contradiction on the basis of the technical common sense of those skilled in the art.

Hereinafter, examples of the method and the composition of the present invention will be described. It shall be understood that various other embodiments can be carried out in light of the general description mentioned above.

### EXAMPLES

### Example 1: IL-12 Fusion Protein has Long Systemic Half-Life Before Protease Cleavage

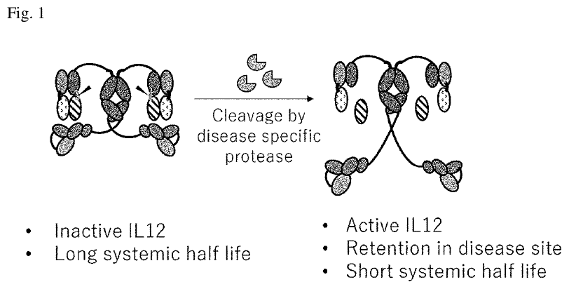

In order to deliver cytokines, such as Interleukin-12 (IL-12), at high dose with low or negligible systemic toxicity, the present inventors have developed an IL-12 fusion protein having protease cleavable linkers. The IL-12 fusion protein remains in an inactivated state until otherwise exposed to an environment with high concentrations of proteases which activates the protein by cleaving the protease cleavage site within the protein. Once cleaved, IL-12 no longer binds to the protein, restoring its physiological activity to bind to its receptor and becomes capable of exerting its biological activity to activate IL-12 receptor signalling. The presently described IL-12 fusion protein allows for target site specific activation that harness the naturally high concentrations of proteases in certain environments, e.g. tumour or inflammatory tissues. The presently described IL-12 fusion protein in the uncleaved state (inactive) has a long systemic half-life compared to recombinant IL-12 alone. It has been reported that IL-12 is known to have a very short half-life of approximately 5-10 hours (AACR Journals. 1999 January; 5(1): 9-16). However, when cleaved (active), the IL-12 fusion protein will demonstrate a faster half-life than recombinant IL-12 (FIG. 1).

### 1-1 Preparation of IL-12 Fusion Protein—Monovalent Release Type

Several IL-12 fusion proteins were constructed by fusing IL-12 molecules, comprising the p40 (SEQ ID NO: 939) and p35 (SEQ ID NO: 940) subunits, with IgG-like polypeptides that bind IL-12 via protease-cleavable linkers. IL-12 binding polypeptides employed incorporates Ab1 (WO2010017598), Ab2, (WO2002012500) and Ab3 (WO2000056772). Further, unless otherwise noted, modifications were performed in the Fc region of said protein that abolishes Fc gamma R binding, comprising amino acid mutations L235R/G236R according to the EU numbering.

Three IL-12 fusion proteins, each comprising a monovalent heterodimer of a polypeptide comprising an IL-12 binding domain (anti-IL-12) and a polypeptide comprising a KLH binding domain (anti-KLH), as follows:
a) Monovalent IL-12 release FP1 is a hetero-dimer of a pair of light chain 1 (SEQ ID NO: 944)/heavy chain 1(SEQ ID NO: 949) and light chain 2 (SEQ ID NO: 950)/heavy chain 2 (SEQ ID NO: 951). In heavy chain 1 (SEQ ID NO: 949), cleavable linker (SEQ ID NO: 941) was introduced into elbow hinge region between FP1 VH (SEQ ID NO: 946) and CH1 domains and cleavable linker (SEQ ID: 947) was introduced between Fe and single chain IL-12 (SEQ ID: 962).
b) Monovalent IL12 release FP2 is a hetero dimer of a pair of light chain 1 (SEQ ID NO: 955)/heavy chain 1 (SEQ ID NO: 999) and light chain 2 (SEQ ID NO: 950)/heavy chain 2 (SEQ ID NO: 951). In heavy chain 1 (SEQ ID NO: 999), cleavable linker (SEQ ID NO: 941) was introduced into elbow hinge region between FP2 VH (SEQ ID NO: 957) and CH1 domains and cleavable linker (SEQ ID: 947) was introduced between Fc and single chain IL-12 (SEQ ID: 962).
c) Monovalent IL12 release FP3 is a hetero dimer of a pair of light chain 1 (SEQ ID NO: 958)/heavy chain 1 (SEQ ID NO: 1000) and light chain 2 (SEQ ID NO: 950)/heavy chain 2 (SEQ ID NO: 951). In heavy chain 1 (SEQ ID NO: 1000), cleavable linker (SEQ ID NO: 941) was introduced into elbow hinge region between FP3 VH (SEQ ID NO: 960) and CH1 domains and cleavable linker (SEQ ID: 1051) was introduced between Fc and single chain IL-12 (SEQ ID: 962).

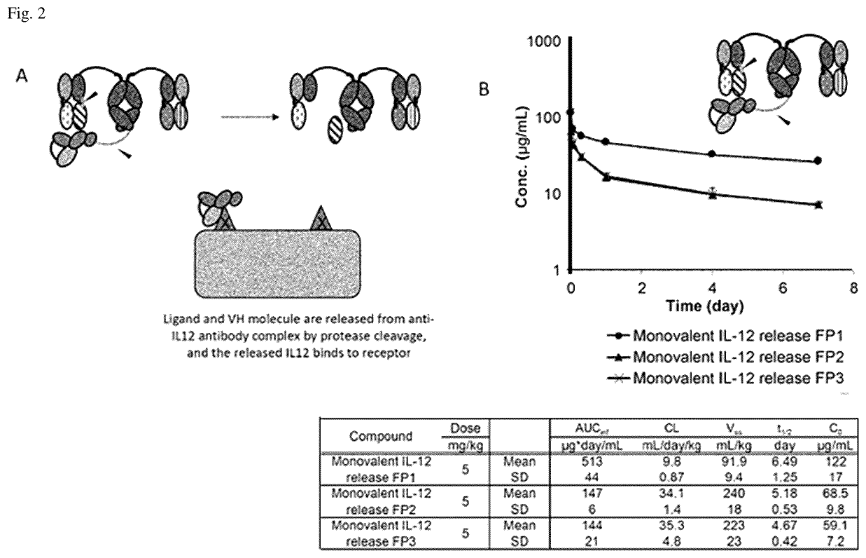

For each of the above, in order to promote hetero-dimerization and precise association of heavy and light chains, knobs-into-holes mutations (Nat. Biotechnol, 1998, 16, 677-681) were introduced in heavy chain CH3 domains and CrossMab technology (PNAS, 2011, 108, 11187-11192) was employed in heavy chain 2 and light chain 2. Heavy chain 1 and heavy chain 2 contain knob mutations (Y349C/T366W) and hole mutations (E356C/T366S/L368A/Y407V), respectively. Light chain 2 was composed of VH domain of anti-KLH with human kappa constant region. Heavy chain 2 was composed of VL domain of anti-KLH and modified IgG1 Fc region. Once the cleavable linker is digested by protease, active IL-12 freely dissociates from the fusion polypeptide and binds to its receptor (FIG. 2A).

Expression vectors of each chain were prepared by a method known to those skilled in the art and expressed using Expi293 (Life Technologies Corp.) by combining each chain as shown in Table 3. Purification of the fusion protein was done using affinity purification by MabSelect SuRe (Cat. No: 17-5438-01, GE Healthcare) followed by size exclusion chromatography using Superdex 200 gel filtration column (Cat. No: 28-9893-35, GE Healthcare). Any aggregates present in the elution from affinity chromatography were removed using size exclusion chromatography.

TABLE 3
Monovalent IL-12 fusion proteins of release
format and sequence IDs of each chain.

| IL-12 fusion | Light | Heavy | Light | Heavy |
|---|---|---|---|---|
| proteins | chain 1 | chain 1 | chain 2 | chain 2 |
| Monovalent | SEQ ID | SEQ ID | SEQ ID | SEQ ID |
| IL-12 release | NO: 944 | NO: 949 | NO: 950 | NO: 951 |
| FP1 | | | | |
| Monovalent | SEQ ID | SEQ ID | SEQ ID | SEQ ID |
| IL-12 release | NO: 955 | NO: 999 | NO: 950 | NO: 951 |
| FP2 | | | | |
| Monovalent | SEQ ID | SEQ ID | SEQ ID | SEQ ID |
| IL-12 release | NO: 958 | NO: 1000 | NO: 950 | NO: 951 |
| FP3 | | | | |

### 1-2 Long Systemic Half-Life of Uncleaved IL-12 Fusion Proteins

In the present invention, the inventors sought to obtain IL-12 fusion proteins that exhibit long systemic half-life in the uncleaved (“inactive”) state before protease digestion but exhibit a short systemic half-life in the cleaved (“active”) state. To ensure that the uncleaved IL-12 fusion protein is not subjected to immediate heparin-mediated elimination which is a usual observation for IL-12 binding molecules such as anti-IL-12 antibodies, fusion proteins comprising various ligand-binding domains that bind to IL-12 were screened as follows.

5 mg/kg of monovalent IL-12 release FP1 to FP3 as described in Table 3 above were intravenously administered into the tail vein of 6-week-old CB17/Icr-Prkdcscid/CrlCrlj female mice (Charles River Laboratories, Japan) and their plasma was successively collected from the jugular vein at the following timepoints: 5 minutes, 1 hour, 7 hours, 1 day, 4 days, and 7 days after administration. The total concentrations of fusion proteins in mouse plasma were measured by LC/ESI-MS/MS. Calibration standards were prepared by serial dilution in mouse plasma. Calibration standard concentrations were 0.195, 0.391, 0.781, 1.56, 3.13, 6.25, 12.5, 25 and 50 microgram (micro g)/mL. A 3 microliter (micro L) of the calibration standards and plasma samples were mixed with 50 micro L of the magnetic beads coated with an anti-human Fc region antibody (in-house). After washing the beads 3 times with 0.05% Tween-20 containing PBS and 1 time with PBS, the samples were mixed with 25 micro L of mixed reagent (8 mmol/L dithiothreitol, 7.5 mol/L urea and 99 ng/mL lysozyme (chicken egg white) in 50 mmol/L ammonium bicarbonate) and incubated for approximately 45 min at 56 degrees C. Then, 2 micro L of 500 mmol/L iodoacetamide was added and incubated for approximately 30 min at 37 degrees C. in the dark. Next, 160 micro L of 0.621 micro g/mL sequencing grade modified trypsin (Cat. No.: V5117, Promega) in 50 mmol/L ammonium bicarbonate was added and incubated at 37 degrees C. overnight. Finally, 5 micro L of 10% trifluoroacetic acid was added to deactivate any residual trypsin. An aliquot of 80 micro L of digestion samples was subjected to analysis by LC/ESI-MS/MS. LC/ESI-MS/MS was performed using Xevo TQ-S triple quadrupole instrument (Waters) equipped with Acquity I-class 2D high-performance liquid chromatography systems (Waters). The antibody-derived tryptic peptide (TLTIQVK) was monitored by the selected reaction monitoring (SRM). SRM transition was m/z 401.755>588.372. The calibration curve of the antibody was constructed by the weighted (1/x2) linear regression using the peak area plotted against the concentrations respectively. The concentrations in mouse plasma were calculated from the calibration curve using the analytical software Masslynx Ver.4.1 (Waters).

Concentration of each antibody was determined by the calibration curve based on the peak area of monitored peptide in calibration samples. Pharmacokinetic parameters were calculated by non-compartmental analysis using Phoenix WinNonlin version 8.0 (Certara USA inc.). The averaged plasma concentrations and parameters of 3 animals are shown in FIG. 2B with their standard deviations.

Monovalent IL-12 release FP1 demonstrated approximately 3-fold lower plasma clearance (9.8 ml/day/kg) than the other 2 fusion proteins, i.e. FP2 (34.1 ml/day/kg) and FP3 (35.3 ml/day/kg) in the same monovalent heterodimer fusion protein format. The above result implies that the heparin binding region of IL-12 in uncleaved (“inactive”) monovalent IL-12 release FP1 was sufficiently masked to prevent heparin binding-mediated elimination.

### 1-3 Preparation of Bivalent IL-12 Fusion Protein of Release and Fusion Formats

Bivalent IL-12 release FP4 is a homodimer of a pair of light chain (SEQ ID NO: 944) and heavy chain (SEQ ID NO: 948). SEQ ID NO: 944 was employed as light chain without modification. In heavy chain, cleavable linker was introduced into elbow hinge region between FP1 VH (SEQ ID NO: 946) and CH1 domains. GS linker (SEQ ID NO: 1001) was inserted in hinge region and single-chain IL-12 (SEQ ID: 962) was attached to C-terminal of Fc domain via cleavable linker (SEQ ID NO: 947). Once these cleavable linkers were digested by proteases, active IL-12 molecules are released (FIG. 3A).

Bivalent IL-12 fusion FP5 is a homodimer of a light chain (SEQ ID NO: 944) and heavy chain (SEQ ID NO: 953). FP1VL-k0 (SEQ ID NO: 944) was employed as light chain without modification. In heavy chain, cleavable linker (SEQ ID NO: 941) was introduced into elbow hinge region between FP1VH (SEQ ID NO: 946) and CH1 domains. GS linker (SEQ ID NO: 1001) was inserted in hinge region and single-chain IL-12 (SEQ ID NO: 962) was attached to C-terminal of Fc domain via GS linker (SEQ ID NO: 963). Once the cleavable linker was digested by protease, active IL-12 molecules that remain fused to the C-terminal of Fc region dissociate from the ligand-binding domain of the fusion protein and bind to their receptors (FIG. 3B).

Expression vectors of each chain were prepared by a method known to those skilled in the art and expressed using Expi293 (Life Technologies Corp.) by combining each chain as shown in Table 4. Purification of fusion proteins was done using affinity purification by MabSelect SuRe (Cat. No: 17-5438-01, GE Healthcare) followed by size exclusion chromatography using Superdex 200 gel filtration column (Cat. No: 28-9893-35, GE Healthcare). Any aggregates present in the elution from affinity chromatography were removed using size exclusion chromatography.

TABLE 4
Bivalent IL-12 fusion proteins of release and
fusion formats and sequence IDs of each chain.

| IL-12 fusion | Light | Heavy | Light | Heavy |
|---|---|---|---|---|
| proteins | chain 1 | chain 1 | chain 2 | chain 2 |
| Bivalent IL-12 | SEQ ID | SEQ ID | — | — |
| release FP4 | NO: 944 | NO: 948 | | |
| Bivalent IL-12 | SEQ ID | SEQ ID | — | — |
| fusion FPS | NO: 944 | NO: 953 | | |

### 1-4 Evaluation of Activity of Bivalent IL-12 Fusion Proteins in Release and Fusion Formats

IL-12 was purified by co-expression of vectors expressing p40 (SEQ ID NO: 939) and p35 (SEQ ID NO: 940) with TEV site followed by (His)₆ tag fused at the C-terminus. Expression vectors of each chain were prepared by a method known to those skilled in the art and expressed using Expi293 (Life Technologies Corp.) by combining each chain. Purification of proteins was done using affinity purification by Ni Sepharose excel (Cat. No: 17-3712-02, GE Healthcare) followed by size exclusion chromatography using Superdex 200 gel filtration column (Cat. No: 28-9893-35, GE Healthcare). Any aggregates present in the elution from affinity chromatography were removed using size exclusion chromatography.

To assess IL-12 bioactivity of bivalent IL-12 fusion proteins of the release and fusion formats with or without MT-SP1 protease treatment, IL-12 luciferase assay was conducted. Briefly, 2.5×10⁴ cells/well IL-12 bioassay cell (Promega, Cat #CS2018A02A) which express human IL-12Rb1, IL-12Rb2, and STAT4, were plated in 96-well plate and incubated overnight. As control, IL-12 was utilised. Bivalent IL-12 fusion proteins of both release and fusion formats were added to the culture plate and incubated for 18 hours. For protease-treated samples, IL-12 and bivalent IL-12 fusion proteins of both release and fusion formats were treated with equimolar concentration of MT-SP1 for 4 hours and serial diluents were prepared. Luciferase activity was detected with Bio-Glo luciferase assay system (Promega, G7940) according to manufacturer's instructions. Luminescence was detected using GloMax (registered trademark) Explorer System (Promega #GM3500). Data analysis was done by Microsoft (registered trademark) Excel (registered trademark) 2013 and the analysed data was plotted using GraphPad Prism 7.

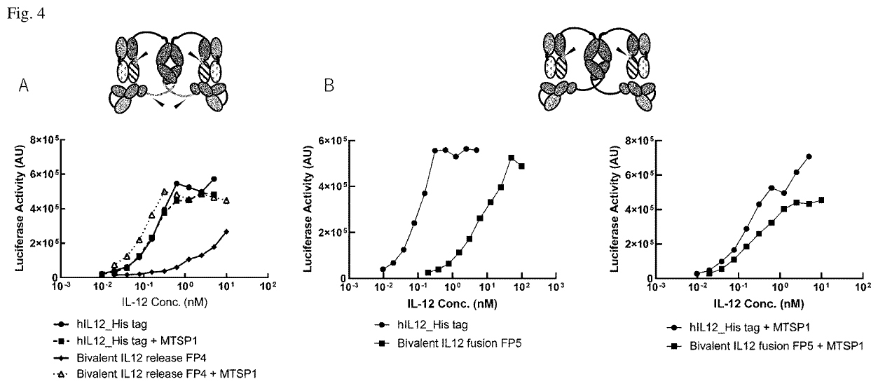

Bivalent IL-12 release FP4, and Bivalent IL-12 fusion FP5 were subjected to the IL-12 luciferase assay. Both variants showed lower IL-12 bioactivity than hIL-12_His tag in the absence of MT-SP1, and the IL-12 bioactivity was recovered to the same level as hIL-12_His tag upon MT-SP1 treatment (FIGS. 4A and 4B).

Based on the above, it has been demonstrated that bivalent IL-12 fusion proteins of both the release and fusion formats lose their ability (i.e., the ability was attenuated) to bind their ligand receptor, i.e. IL-12 receptor, and this binding was inhibited in the uncleaved (inactive) state. However, once cleaved in the presence of protease (active state), the biological activity of the ligand is restored, i.e. IL-12 was capable of binding to its ligand receptor, i.e. IL-12 receptor.

### Example 2: Fusion Format Accumulates in Higher Concentrations within the Tumour and Exhibits Faster Clearance than Release Format

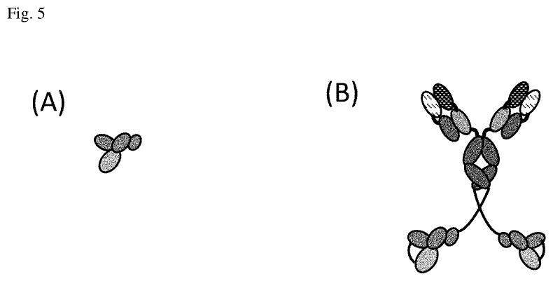

IL-12 fusion proteins in the inactivated state binds to IL-12 and inhibits the ability of IL-12 to bind its receptor. When exposed to proteases, for example, tumour specific proteases, the IL-12 fusion protein is activated, the ability of IL-12 to bind its receptor is restored. As described in Example 1, in their separate protease activated forms, the additional cleavage site between Fc region and IL-12 allows for the full release of IL-12 (release format), while without the additional cleavage site, the IL-12 remains fused to the C-terminal of the Fc region (fusion format). Tumour accumulation and clearance studies were performed using recombinant IL-12 resulting from the activated form of the release format and the activated form of the fusion format IL-12 fusion protein (FIG. 5).

### 2-1 Preparation of Activated Forms of Release Type and Fusion Type IL-12 Fusion Proteins

IL-12 heterodimer protein comprising p40 subunit (SEQ ID NO: 939) and p35 subunit (SEQ ID NO: 1002), each comprising TEV protease recognition site (SEQ ID NO: 1003) and FLAG tag (SEQ ID NO: 1004) was prepared. In addition, KLH Bivalent IL-12 fusion FP6 is prepared, which comprises a homodimer of a light chain (SEQ ID NO: 986) and heavy chain (SEQ ID NO: 1005). SEQ ID NO: 986 was employed as light chain without modifications. In heavy chain (SEQ ID NO: 1005), KLH VH (SEQ ID NO: 994) is fused to constant region (SEQ ID NO: 1006) followed by single-chain IL12 (SEQ ID NO: 962) attached to C-terminal of Fc via GS linker (SEQ ID NO: 963).

KLH Bivalent IL-12 fusion FP7 is prepared, which comprises a homodimer of a light chain (SEQ ID NO: 986) and heavy chain (SEQ ID NO: 1007). SEQ ID NO: 986 was employed as light chain without modifications. In heavy chain (SEQ ID NO: 1007), KLH VH (SEQ ID NO: 994) is fused to constant region, C6 (SEQ ID NO: 1006) followed by single-chain IL12 (SEQ ID NO: 1008) attached to C-terminal of Fc via GS linker (SEQ ID NO: 963).

Expression vectors of each chain were prepared by a method known to those skilled in the art and expressed using Expi293 (Life Technologies Corp.) by combining each chain as shown in Table 5. Purification of IL-12 was done using affinity purification by Anti-FLAG M2 resin followed by size exclusion chromatography using Superdex 200 gel filtration column (Cat. No: 28-9893-35, GE Healthcare). Purification of fusion protein was done using affinity purification by MabSelect SuRe (Cat. No: 17-5438-01, GE Healthcare) followed by size exclusion chromatography using Superdex 200 gel filtration column (Cat. No:28-9893-35, GE Healthcare). Any aggregates present in the elution from affinity chromatography were removed using size exclusion chromatography.

TABLE 5
Activated bivalent IL-12 fusion proteins of
fusion format and sequence IDs of each chain.

| KLH bivalent | | | | |
|---|---|---|---|---|
| IL-12 fusion | Light | Heavy | Light | Heavy |
| proteins | chain 1 | chain 1 | chain 2 | chain 2 |
| KLH Bivalent | SEQ ID | SEQ ID | — | — |
| IL-12 fusion | NO: 986 | NO: 1005 | | |
| FP6 | | | | |
| KLH Bivalent | SEQ ID | SEQ ID | | |
| IL-12 fusion | NO: 986 | NO: 1007 | | |
| FP7 | | | | |

### 2-2 Evaluation of Tumour Accumulation of Bivalent IL-12 Fusion Proteins In Vivo Using Tumour Bearing Mice

### 2-2-1 In Vivo Testing Using Human T Cell Injected Tumour Bearing Mice Model

The LS1034 human colorectal carcinoma cell line was obtained from American Type Culture Collection. Cells were cultured in RPMI-1640 medium (SIGMA) plus 0.45% D-glucose (SIGMA), 10 mM HEPES (SIGMA), 1 mM Sodium Pyruvate (Gibco) with 10% fetal bovine serum (FBS; Nichirei Biosciences). NOD/ShiJic-scidJcl female mice of 6 weeks of age were purchased from CLEA Japan, Inc and were acclimated for 2 weeks before the inoculation. LS1034 cells in log phase growth were harvested and washed with Hank's balanced salt solution (HBSS; SIGMA), resuspended in 50% HBSS and 50% Matrigel (CORNING) at a concentration of 5×10⁷ cells/mL. Mice were subcutaneously inoculated with 1×10⁷ LS1034 cells in 200 micro L of HBSS:Matrigel (1:1). When mean tumour volume reached about 100-300 mm³ (7 days after inoculation), mice were randomized into groups based on tumour volume and body weight. Tumour volume was measured with caliper, and tumour volume was calculated as ½×1×w², l=length, w=width. 1 day after the randomization, mice were inoculated with 3×10⁷ human T cells. After T cell inoculation, 11.3 pmol of IL-12 or 11.3 pmol of KLH-Bivalent IL-12 fusion FP6 were intratumorally administered 3 times a week for 2 weeks. Tumour was resected on 12 and 13 days after first treatment. 2-2-2 Preparation of tumour lysate and tumour interstitial fluid

One-fourth of tumour sections were placed on the tube with mesh and centrifuged at 400×g at 4 degrees C. for 10 minutes. The collected fluid samples were centrifuged again at 10,000×g at 4 degrees C. for 10 minutes, then supernatants were kept as tumour interstitial fluid. Add 9 times the weight of the Lysis buffer (50 mM Tris, 150 mM NaCl, 0.5% Sodium deoxycholate, 2% NP-40 at pH8.0 with protease inhibitor cocktail) into the remaining tumour sections and homogenized by TissueLyser II (Qiagen). The homogenates were centrifuged at 14,000 rpm at 4 degree C. for 10 minutes and the supernatants were kept as 10% tumour lysate.

### 2-2-3 Measurement of IL-12 Concentration in Tumour Lysate and Tumour Interstitial Fluid by Electrochemiluminescence (ECL)

The concentration of IL-12 in tumour lysate and tumour interstitial fluid were measured by electrochemiluminescence (ECL). MSD GOLD 96-well Streptavidin SECTOR Plates (Meso Scale Discovery) were blocked with assay buffer (PBS-T+1% BSA from Roche and Sigma, respectively) for 1 hour at room temperature. Anti-IL-12 immobilized plates were prepared by dispensing biotinylated anti-IL-12 polyclonal antibody (R&D Systems) onto blocked plates and incubating in assay buffer for 1 hour at room temperature. Calibration curve samples of IL-12 and KLH-Bivalent IL-12 fusion FP6, tumour lysate samples, and tumour interstitial fluid samples were prepared by serial dilutions, of 100-fold, or more, were prepared. Subsequently, the samples were added onto an anti-IL-12-immobilized plate and allowed to bind for 1 hour at room temperature before washing. Next, SULFO TAG labelled anti-IL-12 antibody (U-CyTech biosciences, SULFO TAG labelled using MSD GOLD SULFO-TAG NHS-Ester) was added and the plate was incubated for 1 hour at room temperature before washing. Read Buffer T (x4) (Meso Scale Discovery) was immediately added to the plate and signal was detected by SECTOR Imager 2400 (Meso Scale Discovery). Recombinant IL-12 or KLH-Bivalent IL-12 fusion protein concentration was calculated based on the response of the calibration curve using the analytical software SOFTmax PRO (Molecular Devices). The tumour lysate and tumour interstitial fluid concentrations of recombinant IL-12 and KLH-Bivalent IL-12 fusion FP6 measured by

2-2-4 Tumour Retention of IL-12 and KLH-Bivalent IL-12 Fusion FP6 in Mice after Intra-Tumour Injection

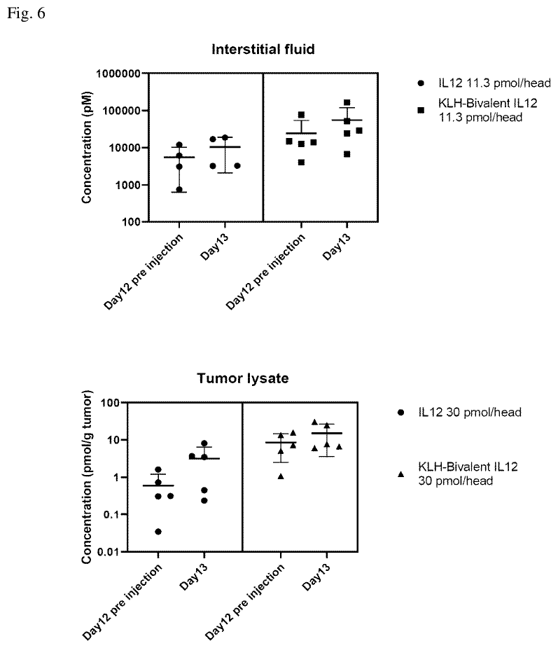

this method is shown in FIG. 6.

Tumour retention after intra-tumour injection was evaluated. KLH-Bivalent IL-12 fusion FP6 showed higher concentration than recombinant IL-12 in both tumour lysate and interstitial fluid at pre-dose and 1 day after last dosing. FIG. 6 illustrates IL-12 concentrations in tumour lysate and tumour interstitial fluid of recombinant IL-12 or KLH-Bivalent IL12 fusion FP6 in human T cell injected LS1034 tumour bearing mice model after intra-tumour injection, repeated for a total of six times.

### 2-3 Evaluation of Pharmacokinetics of Bivalent IL-12 Fusion Proteins in Cynomolgus Monkey

### 2-3-1 Measurement of KLH-Bivalent IL-12 Fusion FP6 Concentration in Cynomolgus Monkey Plasma by ELISA

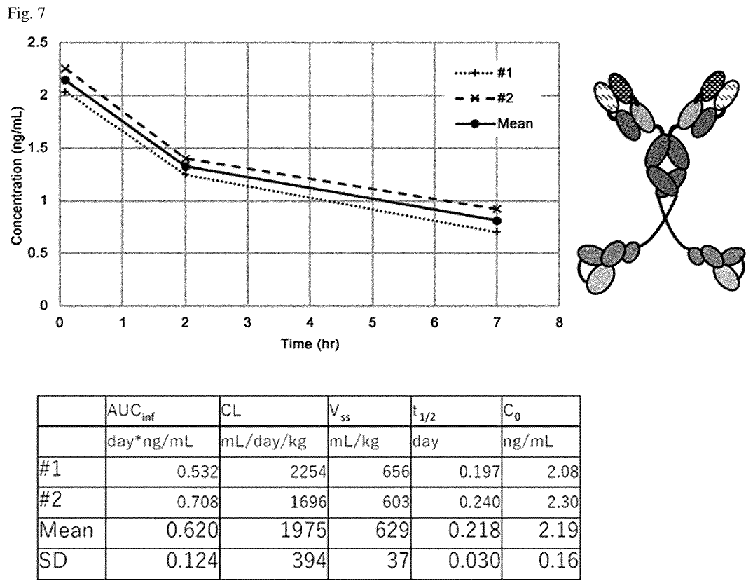

The concentration of KLH-Bivalent IL-12 fusion FP6 in plasma derived from cynomolgus monkey was measured by IL-12 High Sensitivity Human ELISA kit (Abcam) according to the manufacturer's instruction. KLH-Bivalent IL-12 fusion FP6 concentration was calculated based on the response of the calibration curve using the analytical software SOFTmax PRO (Molecular Devices). The time course of KLH-Bivalent IL-12 fusion FP6 concentration in plasma measured by this method is shown in FIG. 7.

### 2-3-2 In Vivo Test Using Cynomolgus Monkey

The pharmacokinetics of KLH-Bivalent IL-12 fusion FP6 was assessed in cynomolgus monkey (FIG. 7). KLH-Bivalent IL-12 fusion FP6 (0.48 mg/mL) was administrated at a single dose of 2.5 mL/kg into the cephalic vein of the forearm. Blood was collected at 5 minutes, 2 hours and 7 hours after administration. The collected blood was centrifuged immediately at 1700×g at 4 degrees C. for 5 minutes to separate the plasma. The separated plasma was stored at below −70 degrees C. until measurement.

### 2-3-3 Pharmacokinetics of KLH-Bivalent IL-12 Fusion FP6 in Cynomolgus Monkey

Pharmacokinetic profiles of KLH-Bivalent IL-12 fusion FP6 in cynomolgus monkey was evaluated. FIG. 7 illustrates the time course of plasma KLH-Bivalent IL-12 fusion FP6 concentration after intravenous administration in cynomolgus monkey. KLH-Bivalent IL-12 fusion FP6 was eliminated rapidly and the clearance was 1975 mL/day/kg which is 13-times faster than recombinant IL-12 clearance reported in literature which is 6.23 mL/hour/kg (150 mL/day/kg) (Pharmacology 2010; 85:319-327).

As demonstrated in the tumour bearing mice model, higher concentrations of the active form of the fusion format IL-12 fusion protein were detected when compared to recombinant IL-12 of the release format IL-12 fusion protein (FIG. 6). Despite the higher concentrations accumulated within the tumour microenvironment, the activated form of the fusion format IL-12 fusion protein demonstrated considerably much faster clearance than recombinant IL-12 (FIG. 7). It is therefore envisioned that fusion format IL-12 fusion protein will exhibit lower systemic toxicity compared to the release format IL-12 fusion protein.

### Example 3: Engineered VH/VL Interface Promotes Dissociation of VH/VL and IL-12 Upon Protease Cleavage

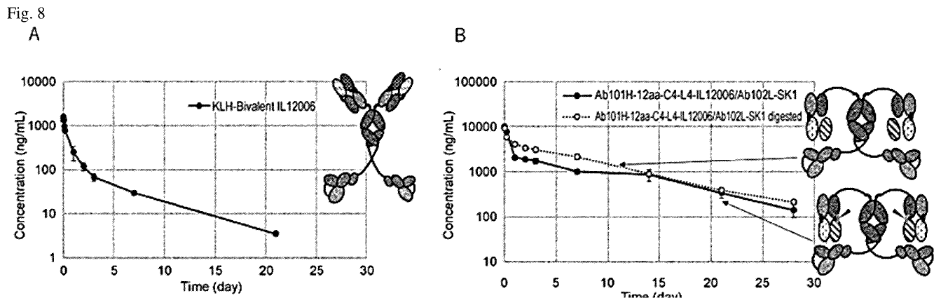

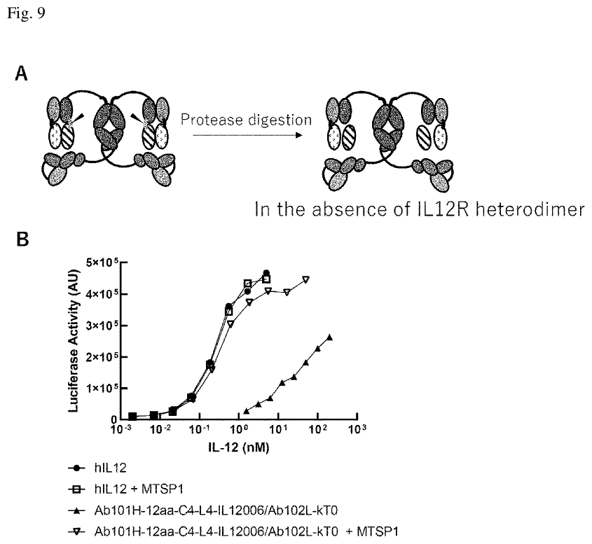

In its inactivated form, the IL-12 ligand binds to the ligand-binding domain of the fusion protein, i.e. the IL-12 ligand binds to the variable region of the fusion protein and the biological activity of the ligand to bind its ligand receptor is inhibited. In a particular example, upon cleavage by protease in the target tissue environment, the protease cleavage site near the boundary of VH and CH1 is disrupted, resulting in the release of VH. The release of VH in turn disrupts the binding between IL-12 and the ligand-binding domain of the fusion protein, thus releasing the IL-12. Similar to our observation of rapid elimination in cynomolgus monkey (Example 2), KLH bivalent fusion FP7 showed a clearance of 335 ml/day/kg in SCID mice (FIG. 8A). Meanwhile, it was observed that the activated form of the IL-12 fusion protein had a clearance that was comparable to its inactive form (FIG. 8B). It was hypothesised that this phenomenon was observed potentially as the VH domain, VL domain and IL-12 moiety may exhibit avidity and did not fully dissociate after protease cleavage (FIG. 9A), which subsequently affected the pharmacokinetics of the IL-12 fusion protein, which did not exhibit higher clearance levels. This is plausible as the heparin binding region contributes to fast clearance of the activated fusion protein. In the particular case of the variable region of the presently described fusion proteins except FP2 and FP3, the heparin binding region is in close proximity with the IL-12 binding epitope, thus when IL-12 remains bound due to incomplete release, clearance of the protein may not be so fast. Meanwhile, the activity of the activated form of the IL-12 fusion protein remains capable of binding to IL-12 receptor and activates IL-12 signalling to the same extent as recombinant IL-12 (FIG. 9B).

To break any remaining avidity between VH domain and VL domain that may affect the release of IL-12, engineering of the interface between VH and VL was performed to reduce the association between VH and VL. Predominantly amino acids in the FR regions making up the VH/VL interface have been modified to increase VH release tendency after protease cleavage. Further, the inventors also discovered that additionally modifying amino acid(s) within the CDR region was able to promote dissociation of VH without disrupting IL-12 binding to the variable region of the IL-12 fusion protein.

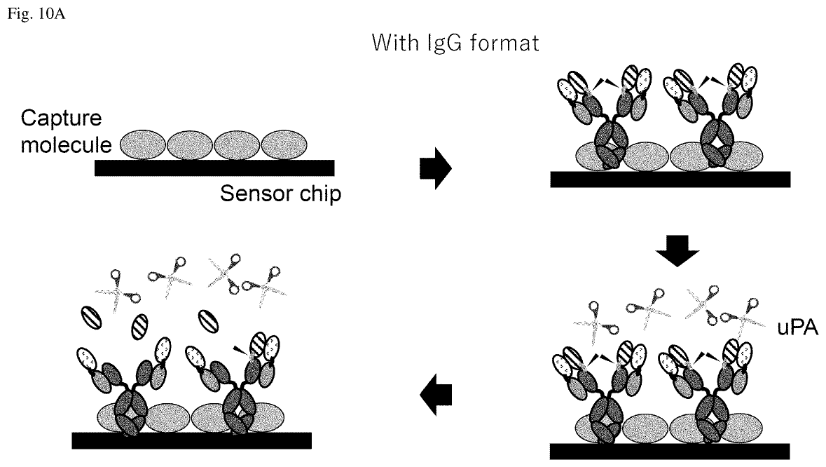

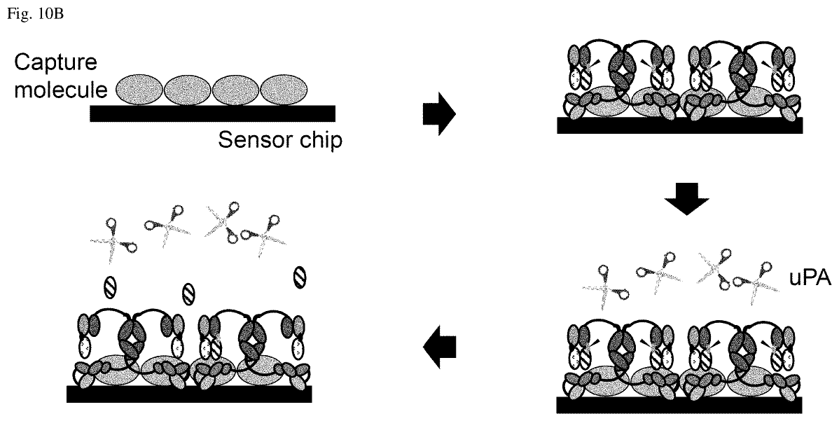

Screening for amino acid modifications that promote dissociation of VH or VL from fusion protein, otherwise also referred as VH release or VH dissociation, were performed first in bivalent fusion protein comprising an IgG antibody having a protease cleavage site near the boundary of VH and CH1 (FIG. 10A) and subsequently in the bivalent fusion protein fusion format (FIG. 10B) described in Examples 1 and 2. The effect of amino acid modifications to improve VH release after protease cleavage was evaluated in vitro. In vitro VH release assay was conducted using Biacore T200 instrument (Cytiva) (FIGS. 10A and 10B). Capture molecule was first immobilized onto all flow cells (FCs) of a sensor chip using amine coupling kit (Cytiva). Next, either the protease cleavable IgG antibodies mentioned above or the protease cleavable bivalent fusion proteins described above were captured onto the flow cells of the sensor surface via the capture protein. This is followed by an injection with recombinant human u-Plasminogen Activator/Urokinase (human uPA, huPA) (R&D Systems; 1310-SE-010) or buffer across the flow cells. Further, the binding affinity of ligand with the antigen binding domain or ligand binding domain was evaluated to ensure that the amino acid modifications performed to improve VH release did not impact the ability of the antigen binding domain or ligand binding domain to neutralise the ligand. Finally, the clearance levels of protease activated bivalent fusion protein comprising the amino acid modifications to improve VH release was evaluated.

3-1 Preparation of Anti-IL-12 Antibodies Comprising Modifications within the VH/VL Interface

Anti-IL-12 antibody, Ab1 (Ab101H-12aa-C8/Ab102L-SK1) is a homodimer made up of a light chain (SEQ ID NO: 1009) and heavy chain (SEQ ID NO: 1010). In heavy chain (SEQ ID NO: 1010), VH, Ab101H (SEQ ID NO: 1011) is fused to constant region, C8 (SEQ ID NO: 1087) via a cleavable linker, 12aa (SEQ ID NO: 941). VH release promoting amino acid modifications were done at either one or more sites of V37, G44, L45, W47, Y91, W103 of Ab101H VH and A43, P44, L46, Y49, Y87 and F98 of Ab102 VL. In some instances, amino acid modifications were also modified in the CDR sites present within the VH/VL interface, e.g. 100a.

Expression vectors of each chain were prepared by a method known to those skilled in the art and expressed using Expi293 (Life Technologies Corp.) and purified by ProA purification method.

TABLE 6
Anti-IL-12 antibody and sequence IDs of each chain.

| Anti-1L-12 | Light | Heavy | Light | Heavy |
|---|---|---|---|---|
| antibody | chain 1 | chain 1 | chain 2 | chain 2 |
| Anti-IL12 | SEQ ID | SEQ ID | — | — |
| antibody Ab1 | NO: 1009 | NO: 1010 | | |

### 3-2 Screening of VH Release Amino Acid Modifications Using Anti-IL-12 Antibody (Ab1) Variants

### 3-2-1 VH Release Evaluation for Single Mutants:

Single amino acid modifications (“single mutants”) were introduced in the VH/VL interface of anti-IL-12 antibody, Ab1, and VH release tendency was measured. VH release for single mutants were determined at 37 degrees C. using Biacore T200 instrument (Cytiva). Anti-human Fc antibody (Cytiva) was immobilized onto all flow cells (FCs) of a CM4 sensor chip using amine coupling kit (Cytiva). All antibodies were prepared in HBS-EP+buffer. Each antibody was captured onto the sensor surface by anti-human Fc antibody. Antibodies were captured at flow cells—FC2, FC3, or FC4 to a level of 200 RU, and then followed by 300 seconds injection of 500 nM of recombinant human u-Plasminogen Activator/Urokinase (human uPA, huPA) (R&D Systems; 1310-SE-010) or buffer across all FCs. Sensor surface was regenerated each cycle with 3M MgCl₂. The RU value at 10 seconds before the sample injection onto flow cells—FC2, FC3, or FC4 ended was adopted as the final response for each antibody. The percentage reduction in RU was calculated using the following formula:

$$\frac{\begin{matrix}Final response of buffer injection - \\ Final response of uPA injection\end{matrix}}{Final response of buffer injectíon} \times 100$$

As VH corresponds to 20% of the molecular weight of an IgG molecule, 20% reduction in response implies 100% of VH has been released.

$$VH release \% = \% reduction in RU \times 100 / 20$$

Many single mutations in the VH/VL interface led to an increase in VH release tendency compared to no modifications (FIG. 11A, Table 7).

TABLE 7
VH release % for single mutants of Ab1

| Variant | % reduction in RU | % VH release |
|---|---|---|
| W47A VH/— | 7% | 35.0 |
| W47L VH/— | 6% | 30.0 |
| W47M VH/— | 7% | 35.0 |
| Y91A VH/— | 7.30% | 36.5 |
| Y91L VH/— | 6.70% | 33.5 |
| Y91M VH/— | 7.10% | 35.5 |
| W103A VH/— | 8.20% | 41.0 |
| W103L VH/— | 7.40% | 37.0 |
| W103M VH/— | 7.60% | 38.0 |
| V37S VH/— | 8.10% | 40.5 |
| V37Q VH/— | 6.20% | 31.0 |
| G44Q VH/— | 4.80% | 24.0 |
| L45A VH/— | 6.10% | 30.5 |
| L45Q VH/— | 6.40% | 32.0 |
| F100aI VH/— | 5.50% | 27.5 |
| —/Y49A VL | 5.70% | 28.5 |
| —/Y87A VL | 7.20% | 36.0 |
| —/Y87L VL | 8.00% | 40.0 |
| —/Y87M VL | 8.10% | 40.5 |
| —/F98A VL | 10.20% | 51.0 |
| —/F98L VL | 9.30% | 46.5 |
| —/F98M VL | 8.10% | 40.5 |
| —/A43Q VL | 7.40% | 37.0 |
| —/P44A VL | 9.10% | 45.5 |
| —/P44S VL | 10.10% | 50.5 |
| —/P44Q VL | 10.00% | 50.0 |
| —/L46E VL | 9.50% | 47.5 |
| —/L46Q VL | 9.40% | 47.0 |
| —/— | 4.65% | 23.3 |

### 3-2-2 VH Release Evaluation for Combination Mutants

Several mutations were selected based on the binding kinetics of the single mutants towards IL-12 (Table 9) and combinations of the selected mutations were performed (“combination mutants”). Binding kinetics of single mutants and combination mutants were evaluated to ensure that the mutations do not affect binding kinetics of the antibody to IL-12 (Example 3-3). VH release for combination mutants were determined at 37 degrees C. using Biacore T200 instrument (Cytiva). Protein A/G (PIERCE) was immobilized onto all flow cells (FCs) of a CM4 sensor chip using amine coupling kit (Cytiva). All antibodies and analytes were prepared in HBS-EP+buffer. Each antibody was captured onto the sensor surface by Protein A/G. Antibodies were captured at flow cells—FC2, FC3, or FC4 to a level of 200 RU, and then followed by 300 seconds injection of 1 uM of recombinant human uPA or buffer across all FCs. Sensor surface was regenerated each cycle with 10 mM Glycine-HCl, pH 1.5. The RU value at 5 seconds before the sample injection onto flow cells—FC2, FC3, or FC4 ended was adopted as the final response for each antibody. The percentage reduction in RU was calculated using the following formula:

$$\frac{\begin{matrix}Final response of buffer injection - \\ Final response of uPA injection\end{matrix}}{Final response of buffer injectíon} \times 100$$

As VH corresponds to 20% of the molecular weight of an IgG molecule, 20% reduction in response implies 100% of VH has been released.

$$VH release \% = \% reduction in RU \times 100 / 20$$

Many combinations of VH-VL interface mutations have shown increase in VH release tendency compared to no modifications (FIG. 11B, Table 8).

TABLE 8
VH release % for combination mutants of Ab1

| | % reduction | % VH |
|---|---|---|
| Variant | in RU | release |
| Y91A VH/A43Q, Y49A VL | 12.24 | 61.2 |
| Y91A VH/P44A, Y49A VL | 7.5 | 37.5 |
| Y91A VH/L46Q, Y49A VL | 14.47 | 72.35 |
| Y91A VH/Y49A, Y87L VL | 8.95 | 44.75 |
| Y91M VH/A43Q, Y49A VL | 13.54 | 67.7 |
| Y91M VH/P44A, Y49A VL | 7.72 | 38.6 |
| Y91M VH/L46Q, Y49A VL | 11.64 | 58.2 |
| Y91M VH/Y49A, Y87L VL | 10.7 | 53.5 |
| Y91M VH/Y49A, F98L VL | 8.02 | 40.1 |
| W103L VH/A43Q, Y49A VL | 9.06 | 45.3 |
| W103L VH/P44A, Y49A VL | 5.45 | 27.25 |
| W103L VH/L46Q, Y49A VL | 15.12 | 75.6 |
| W103L VH/Y49A, Y87L VL | 12.29 | 61.45 |
| W103I VH/A43Q, Y49A VL | 13.65 | 68.25 |
| W103I VH/P44A, Y49A VL | 9.15 | 45.75 |
| W103I VH/L46Q, Y49A VL | 14.9 | 74.5 |
| W103M VH/A43Q, Y49A VL | 8.21 | 41.05 |
| W103M VH/P44A, Y49A VL | 6.5 | 32.5 |
| W103M VH/L46Q, Y49A VL | 14.95 | 74.75 |
| W103M VH/Y49A, Y87L VL | 14.29 | 71.45 |
| V37S VH/A43Q, Y49A VL | 12.74 | 63.7 |
| V37S VH/P44A, Y49A VL | 8.97 | 44.85 |
| V37S VH/L46Q, Y49A VL | 17.83 | 89.15 |
| V37S VH/Y49A, Y87L VL | 12.07 | 60.35 |
| V37S VH/Y49A, F98L VL | 11.18 | 55.9 |
| L45Q VH/A43Q, Y49A VL | 8.64 | 43.2 |
| L45Q VH/P44A, Y49A VL | 5.43 | 27.15 |
| L45Q VH/L46Q, Y49A VL | 7.87 | 39.35 |
| L45Q VH/Y49A, Y87L VL | 5.87 | 29.35 |
| L45Q VH/Y49A, F98M VL | 10.71 | 53.55 |
| F100aI VH/A43Q, Y49A VL | 14.79 | 73.95 |
| F100aI VH/P44A, Y49A VL | 11.32 | 56.6 |
| F100aI VH/L46Q, Y49A VL | 5.96 | 29.8 |
| F100aI VH/Y49A, Y87L VL | 10.93 | 54.65 |
| F100aI VH/Y49A, F98L VL | 7.62 | 38.1 |
| Y91M VH/A43Q, P44A, Y49A VL | 2.53 | 12.65 |
| Y91M VH/A43Q, L46Q, Y49A VL | 18.13 | 90.65 |
| Y91M VH/L46Q, Y49A, Y87M VL | 11.74 | 58.7 |
| V37S VH/L46Q, Y49A, Y87M VL | 13.5 | 67.5 |
| F100aI VH/A43Q, L46Q, Y49A VL | 5.97 | 29.85 |
| F100aI VH/L46Q, Y49A, Y87M VL | 12.24 | 61.2 |
| V37S, L45Q VH/A43Q, Y49A VL | 8.54 | 42.7 |
| V37S, Y91M VH/A43Q, Y49A VL | 13.53 | 67.65 |
| V37S, F100aI VH/A43Q, Y49A VL | 14.15 | 70.75 |
| V37S, W103M VH/A43Q, Y49A VL | 13.89 | 69.45 |
| V37S, Y91M VH/L46Q, Y49A VL | 4.87 | 24.35 |
| V37S, F100aI VH/L46Q, Y49A VL | 13.42 | 67.1 |
| V37S, L45Q VH/Y49A, Y87M VL | 13.54 | 67.7 |
| —/— | 2 | 10 |

### 3-3 Single and Combination Mutations do not Affect Binding Kinetics Between Anti-IL-12 Antibody and IL-12

The affinity of anti-IL-12 antibodies binding to human IL-12 at pH 7.4 were determined at 37 degrees C. using Biacore T200 instrument (Cytiva). Anti-human Fc antibody (Cytiva) was immobilized onto all flow cells (FCs) of a CM4 sensor chip using amine coupling kit (Cytiva). All antibodies and analytes were prepared in ACES pH 7.4 containing 20 mM ACES, 150 mM NaCl, 0.05% Tween 20, 0.005% NaN₃. Each antibody was captured onto the sensor surface by anti-human Fc antibody. Antibody capture levels were aimed at 50 response unit (RU). 5 nM of human IL-12 were injected as analyte, followed by dissociation. Sensor surface was regenerated each cycle with 3M MgCl₂. Binding affinity were determined by processing and fitting the data to 1:1 binding model using Biacore T200 Evaluation software (Cytiva). Table 9 shows the binding kinetics towards IL-12 of single mutants and combination mutants comprising of at least one amino acid modification to promote VH dissociation. Based on the results in Table 9, it is observed that binding kinetics of anti-IL-12 antibody AbI variants towards IL-12 were not drastically changed compared to control (−/−).

TABLE 9
Binding kinetics of single and combination
mutants of Ab1 variants towards IL-12

| Variant | Kon | Koff | KD (M) |
|---|---|---|---|
| —/— | 3.38E+06 | 3.2E−03 | 9.48E−10 |
| W47A VH/— | — | — | — |
| W47L VH/— | — | — | — |
| W47M VH/— | — | — | — |
| Y91A VH/— | 5.00E+06 | 4.73E−03 | 9.48E−10 |
| Y91L VH/— | 4.32E+06 | 5.22E−03 | 1.21E−09 |
| Y91M VH/— | 3.89E+06 | 3.22E−03 | 8.27E−10 |
| W103A VH/— | 1.55E+07 | 5.13E−03 | 3.31E−10 |
| W103L VH/— | 7.71E+06 | 4.23E−03 | 5.49E−10 |
| W103M VH/— | 7.29E+06 | 4.60E−03 | 6.31E−10 |
| V37S VH/— | 4.23E+06 | 3.34E−03 | 7.89E−10 |
| V37Q VH/— | 3.75E+06 | 5.56E−03 | 1.48E−09 |
| G44Q VH/— | 3.84E+06 | 3.04E−03 | 7.93E−10 |
| L45A VH/— | 4.41E+06 | 5.59E−03 | 1.27E−09 |
| L45Q VH/— | 4.37E+06 | 5.67E−03 | 1.3E−09 |
| F100aI VH/— | 1.09E+07 | 6.23E−03 | 5.71E−10 |
| —/Y49A VL | 3.10E+06 | 5.39E−03 | 1.74E−09 |
| —/Y87A VL | 4.26E+06 | 5.13E−03 | 1.21E−09 |
| —/Y87L VL | 4.19E+06 | 6.10E−03 | 1.46E−09 |
| —/Y87M VL | 3.43E+06 | 4.19E−03 | 1.22E−09 |
| —/F98A VL | 4.90E+06 | 5.03E−03 | 1.03E−09 |
| —/F98L VL | 4.71E+06 | 2.35E−03 | 4.99E−10 |
| —/F98M VL | 3.93E+06 | 2.69E−03 | 6.85E−10 |
| —/A43Q VL | 3.50E+06 | 3.28E−03 | 9.37E−10 |
| —/P44A VL | 4.19E+06 | 3.59E−03 | 8.58E−10 |
| —/P44S VL | 5.68E+06 | 6.67E−03 | 1.17E−09 |
| —/P44Q VL | 5.51E+06 | 5.18E−03 | 9.4E−10 |
| —/L46E VL | — | — | — |
| —/L46Q VL | 3.44E+06 | 5.57E−03 | 1.62E−09 |
| Y91A VH/A43Q, Y49A VL | 4.04E+06 | 7.73E−03 | 1.91E−09 |
| Y91A VH/P44A, Y49A VL | 6.11E+06 | 1.42E−02 | 2.32E−09 |
| Y91A VH/L46Q, Y49A VL | 8.44E+06 | 3.35E−02 | 3.97E−09 |
| Y91A VH/Y49A, Y87L VL | 6.80E+06 | 1.92E−02 | 2.82E−09 |
| Y91M VH/A43Q, Y49A VL | 3.83E+06 | 7.17E−03 | 1.87E−09 |
| Y91M VH/P44A, Y49A VL | 5.05E+06 | 9.26E−03 | 1.83E−09 |
| Y91M VH/L46Q, Y49A VL | 4.79E+06 | 1.84E−02 | 3.83E−09 |
| Y91M VH/Y49A, Y87L VL | 3.47E+06 | 1.08E−02 | 3.1E−09 |
| Y91M VH/Y49A, F98L VL | 4.47E+06 | 4.78E−03 | 1.07E−09 |
| W103L VH/A43Q, Y49A VL | 6.84E+06 | 7.64E−03 | 1.12E−09 |
| W103L VH/P44A, Y49A VL | 1.02E+07 | 1.37E−02 | 1.34E−09 |
| W103L VH/L46Q, Y49A VL | 1.26E+07 | 4.85E−02 | 3.86E−09 |
| W103L VH/Y49A, Y87L VL | 5.98E+06 | 9.03E−03 | 1.51E−09 |
| W103I VH/A43Q, Y49A VL | 4.52E+06 | 7.25E−03 | 1.60E−09 |
| W103I VH/P44A, Y49A VL | 7.58E+06 | 1.19E−02 | 1.57E−09 |
| W103I VH/L46Q, Y49A VL | 9.99E+06 | 4.85E−02 | 4.86E−09 |
| W103M VH/A43Q, Y49A VL | 5.86E+06 | 8.50E−03 | 1.45E−09 |
| W103M VH/P44A, Y49A VL | 7.54E+06 | 8.03E−03 | 1.07E−09 |
| W103M VH/L46Q, Y49A VL | 7.64E+06 | 2.69E−02 | 3.52E−09 |
| W103M VH/Y49A, Y87L VL | 6.29E+06 | 1.13E−02 | 1.79E−09 |
| V37S VH/A43Q, Y49A VL | 4.20E+06 | 6.27E−03 | 1.49E−09 |
| V37S VH/P44A, Y49A VL | 6.42E+06 | 1.90E−02 | 2.96E−09 |
| V37S VH/L46Q, Y49A VL | 4.81E+06 | 1.47E−02 | 3.06E−09 |
| V37S VH/Y49A, Y87L VL | 3.99E+06 | 1.21E−02 | 3.04E−09 |
| V37S VH/Y49A, F98L VL | 4.25E+06 | 4.17E−03 | 9.82E−10 |
| L45Q VH/A43Q, Y49A VL | 4.12E+06 | 9.19E−03 | 2.23E−09 |
| L45Q VH/P44A, Y49A VL | 5.36E+06 | 1.47E−02 | 2.74E−09 |
| L45Q VH/L46Q, Y49A VL | 5.14E+06 | 1.45E−02 | 2.82E−09 |
| L45Q VH/Y49A, Y87L VL | 5.87E+06 | 2.12E−02 | 3.61E−09 |
| L45Q VH/Y49A, F98M VL | 2.74E+06 | 8.96E−03 | 3.27E−09 |
| F100aI VH/A43Q, Y49A VL | 9.64E+06 | 1.29E−02 | 1.33E−09 |
| F100aI VH/P44A, Y49A VL | 1.05E+07 | 1.32E−02 | 1.25E−09 |
| F100aI VH/L46Q, Y49A VL | 1.01E+07 | 3.52E−02 | 3.48E−09 |
| F100aI VH/Y49A, Y87L VL | 1.16E+07 | 2.44E−02 | 2.1E−09 |
| F100aI VH/Y49A, F98L VL | 8.59E+06 | 1.24E−02 | 1.44E−09 |
| Y91M VH/A43Q, P44A, Y49A VL | 5.48E+06 | 9.24E−03 | 1.69E−09 |
| Y91M VH/A43Q, L46Q, Y49A VL | 6.03E+06 | 2.66E−02 | 4.41E−09 |
| Y91M VH/L46Q, Y49A, Y87M VL | 6.76E+06 | 2.59E−02 | 3.83E−09 |
| V37S VH/L46Q, Y49A, Y87M VL | 5.12E+06 | 1.98E−02 | 3.87E−09 |
| F100aI VH/A43Q, L46Q, Y49A VL | 1.35E+07 | 4.80E−02 | 3.56E−09 |
| F100aI VH/L46Q, Y49A, Y87M VL | 1.09E+07 | 4.00E−02 | 3.68E−09 |
| V37S, L45Q VH/A43Q, Y49A VL | 3.87E+06 | 1.21E−02 | 3.13E−09 |
| V37S, Y91M VH/A43Q, Y49A VL | 4.71E+06 | 8.45E−03 | 1.79E−09 |
| V37S, F100aI VH/A43Q, Y49A VL | 1.11E+07 | 1.24E−02 | 1.12E−09 |
| V37S, W103M VH/A43Q, Y49A VL | 8.25E+06 | 6.83E−03 | 8.28E−10 |
| V37S, Y91M VH/L46Q, Y49A VL | 6.13E+06 | 2.11E−02 | 3.44E−09 |
| V37S, F100aI VH/L46Q, Y49A VL | 1.85E+07 | 5.53E−02 | 2.98E−09 |
| V37S, L45Q VH/Y49A, Y87M VL | 5.57E+06 | 1.63E−02 | 2.92E−09 |

3-4 Preparation of IL-12 Fusion Proteins Comprising Modifications within the VH/VL Interface

Bivalent IL-12 fusion protein FP8 (Ab101H-12aa-C4-L4-IL12006/Ab102L-SK1) is a homodimer made up of a light chain (SEQ ID NO: 1009) and heavy chain (SEQ ID NO: 1012). In heavy chain (SEQ ID NO: 1012), VH, Ab101H (SEQ ID NO: 1011) is fused to the N-terminus of constant region, C4 (SEQ ID NO: 970) via a cleavable linker, 12aa (SEQ ID NO: 941) and single chain IL-12, IL12006 (SEQ ID NO: 1008) is fused to the C-terminus of constant region by a GS linker (SEQ ID NO: 963). The heavy chain (SEQ ID NO: 1012) and light chain (SEQ ID NO: 1009) were further modified at the VH/VL interface by performing at least one amino acid modification at the VH/VL interface, and additionally with or without modification(s) in the CDR region. Modifications that had demonstrated high percentage of VH release tendency and minimal or no change in binding kinetics shown in Examples 3-2 and 3-3 above were selected. Several fusion proteins comprising of FP8 and modifications in the VH/VL interface (“Single mutants” or “Combination mutants”) were examined as shown in Table 11A.

Bivalent IL-12 fusion protein FP13 (Ab101H-N0222-C4-L4-IL12v1.KHKE/Ab102L-SK1) is a homodimer made up of a light chain (SEQ ID NO: 1009) and heavy chain (SEQ ID NO: 1088). In the heavy chain (SEQ ID NO: 1088), VH, Ab101H (SEQ ID NO: 1011) is fused to the N-terminus of constant region, C4 (SEQ ID NO: 970) via a cleavable linker, N0222 (SEQ ID NO: 1089) and single chain IL-12, IL12v1.KHKE (SEQ ID NO: 1090) is fused to the C-terminus of constant region by a GS linker (SEQ ID NO: 963). The heavy chain (SEQ ID NO: 1088) and light chain (SEQ ID NO: 1009) were further modified at the VH/VL interface by performing at least one amino acid modification at the VH/VL interface, and additionally with or without modification(s) in the CDR region. Several fusion proteins of FP13 with modifications in the VH/VL interface were examined as shown in Table 11B.

Expression vectors of each chain were prepared by a method known to those skilled in the art and expressed using Expi293 (Life Technologies Corp.) by combining each chain as shown in Table 10. Purification of fusion protein was done using affinity purification by MabSelect SuRe (Cat. No: 17-5438-01, GE Healthcare) followed by size exclusion chromatography using Superdex 200 gel filtration column (Cat. No: 28-9893-35, GE Healthcare). Any aggregates present in the elution from affinity chromatography were removed using size exclusion chromatography.

TABLE 10
Bivalent IL-12 fusion protein and sequence IDs of each chain.

| IL-12 fusion | Light | Heavy | Light | Heavy |
|---|---|---|---|---|
| protein | chain 1 | chain 1 | chain 2 | chain 2 |
| Bivalent IL-12 | SEQ ID | SEQ ID | — | — |
| fusion protein | NO: 1009 | NO: 1012 | | |
| FP8 | | | | |
| Bivalent IL-12 | SEQ ID | SEQ ID | | |
| fusion protein | NO: 1009 | NO: 1088 | | |
| FP13 | | | | |

### 3-5 Screening of VH Release Amino Acid Modifications Using Bivalent IL-12 Fusion Protein (FP8) Variants

VH release for bivalent IL-12 fusion proteins was determined at 37 degrees C. using Biacore T200 instrument (Cytiva). Protein A/G (PIERCE) was immobilized onto all flow cells (FCs) of a CM4 sensor chip using amine coupling kit (Cytiva). All proteins and analytes were prepared in HBS-EP+buffer. Each protein was captured onto the sensor surface by Protein A/G captured at flow cells—FC2, FC3, or FC4 to a level of 500 RU, and then followed by 1800 seconds injection of 400 nM of recombinant human uPA or buffer across all FCs. Sensor surface was regenerated after each cycle with 10 mM Glycine-HCl, pH 1.5. The RU value at 10 seconds before the sample injection onto flow cells—FC2, FC3, or FC4 ended was adopted as the final response for each antibody. The percentage reduction in RU was calculated using the following formula:

$$\frac{\begin{matrix}Final response of buffer injection - \\ Final response of uPA injection\end{matrix}}{Final response of buffer injectíon} \times 100$$

As VH corresponds to 10% of the molecular weight of bivalent IL-12 fusion proteins, 10% reduction in response implies that 100% of VH has been released.

$$VH release \% = \% reduction in RU \times 100 / 10$$

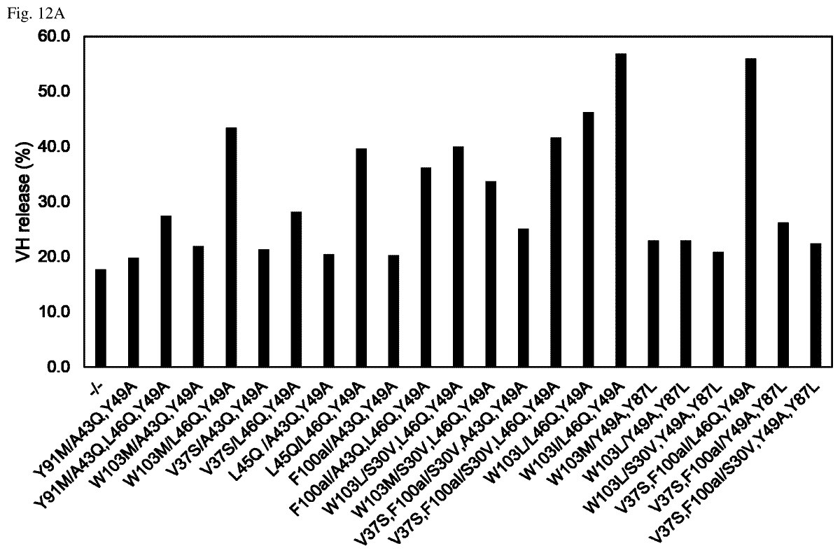

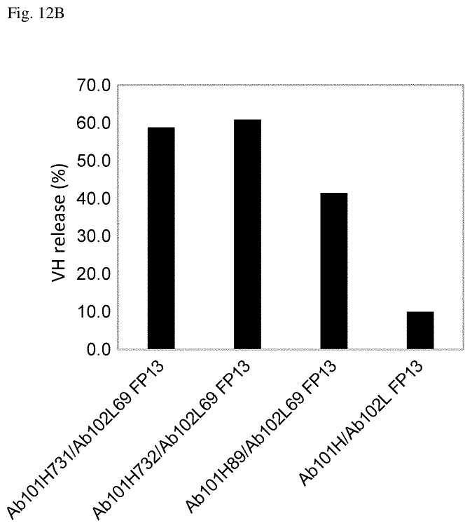

As shown in Tables 11A and 1IB and FIGS. 12A and 12B, all tested variants demonstrated improved VH release tendency.

TABLE 11A
Bivalent IL-12 fusion proteins with VH
release mutations and the VH release %

| | | | VH |
|---|---|---|---|
| | | % re- | re- |
| | | duction | lease |
| Variant name | VH/VL mutation | in RU | % |
| Ab101H/Ab102L | —/— | 1.77 | 17.7 |
| Ab101H10/Ab102L67 | Y91M/A43Q, Y49A | 1.98 | 19.8 |
| Ab101H10/Ab102L80 | Y91M/A43Q, L46Q, Y49A | 2.74 | 27.4 |
| Ab101H15/Ab102L67 | W103M/A43Q, Y49A | 2.19 | 21.9 |
| Ab101H15/Ab102L69 | W103M/L46Q, Y49A | 4.35 | 43.5 |
| Ab101H17/Ab102L67 | V37S/A43Q, Y49A | 2.14 | 21.4 |
| Ab101H17/Ab102L69 | V37S/L46Q, Y49A | 2.81 | 28.1 |
| Ab101H24/Ab102L67 | L45Q/A43Q Y49A | 2.05 | 20.5 |
| Ab101H24/Ab102L69 | L45Q/L46Q, Y49A | 3.96 | 39.6 |
| Ab101H26/Ab102L67 | F100aI/A43Q, Y49A | 2.02 | 20.2 |
| Ab101H26/Ab102L80 | F100aI/A43Q, L46Q, Y49A | 3.61 | 36.1 |
| Ab101H12/Ab102L103 | W103L/S30V, L46Q, Y49A | 4.00 | 40.0 |
| Ab101H15/Ab102L103 | W103M/S30V, L46Q, Y49A | 3.37 | 33.7 |
| Ab101H89/Ab102L101 | V37S, F100aI/S30V, | 2.51 | 25.1 |
| | A43Q, Y49A | | |
| Ab101H89/Ab102L103 | V37S, F100aI/S30V, | 4.16 | 41.6 |
| | L46Q, Y49A | | |
| Ab101H12/Ab102L69 | W103L/L46Q, Y49A | 4.62 | 46.2 |
| Ab101H13/Ab102L69 | W103I/L46Q, Y49A | 5.69 | 56.9 |
| Ab101H15/Ab102L70 | W103M/Y49A, Y87L | 2.29 | 22.9 |
| Ab101H12/Ab102L70 | W103L/Y49A, Y87L | 2.29 | 22.9 |
| Ab101H12/Ab102L130 | W103L/S30V, Y49A, Y87L | 2.09 | 20.9 |
| Ab101H89/Ab102L69 | V37S, F100aI/L46Q, Y49A | 5.60 | 56.0 |
| Ab101H89/Ab102L70 | V37S, F100aI/Y49A, Y87L | 2.62 | 26.2 |
| Ab101H89/Ab102L130 | V37S, F100aI/S30V, | 2.24 | 22.4 |
| | Y49A, Y87L | | |

TABLE 11B
Bivalent IL-12 fusion proteins FP13 with
VH release mutations and the VH release %

| | | | VH |
|---|---|---|---|
| | | % re- | re- |
| | | duction | lease |
| Variant | VH/VL mutation | in RU | % |
| Ab101H/Ab102L | —/— | 1.00% | 10.0% |
| Ab101H89/Ab102L69 | V37S, F100aI/L46Q, Y49A | 4.15% | 41.5% |
| Ab101H731/Ab102L69 | V37S, F100aI, | 5.88% | 58.8% |
| | W103M/L46Q, Y49A | | |
| Ab101H732/Ab102L69 | V37S, F100aI, | 6.09% | 60.9% |
| | W103L/L46Q, Y49A | | |

### 3-6 Preparation of Activated Bivalent IL-12 Fusion Proteins Comprising Amino Acid Modifications at VH/VL Interface

Bivalent IL-12 fusion proteins comprising of amino acid modifications at the VH/VL interface that promotes VH dissociation as described in Table 11A were prepared and additionally subjected to protease treatment. Recombinant Human u-Plasminogen Activator/Urokinase (uPA) (R&D Systems, Inc., 1310-SE-010) was used as the protease. Protease and each bivalent IL-12 fusion protein were reacted in PBS under a condition of 37 degrees C. for 4 hours at 1:1 ratio. The protease was removed from the digestion mixture by pulldown using Ni Sepharose excel resin (Cat. No: 17371201, Cytiva). Table 12 shows the list of variants, VH and VL mutations and sequence IDs.

TABLE 12
Bivalent IL-12 fusion proteins with VH release mutations, sequence
IDs of each chain that were subjected protease digestion

| Variant Name | VH/VL mutation | Heavy chain | Light Chain |
|---|---|---|---|
| Ab101H/Ab102L | —/— | SEQ ID NO: 1012 | SEQ ID NO: 1009 |
| Ab101H15/Ab102L69 | W103M/L46Q, Y49A | SEQ ID NO: 1013 | SEQ ID NO: 1016 |
| Ab101H15/Ab102L103 | W103M/S30V, L46Q, | SEQ ID NO: 1013 | SEQ ID NO: 1017 |
| | Y49A | | |
| Ab101H15/Ab102L70 | W103M/Y49A, Y87L | SEQ ID NO: 1013 | SEQ ID NO: 1018 |
| Ab101H12/Ab102L69 | W103L/L46Q, Y49A | SEQ ID NO: 1014 | SEQ ID NO: 1016 |
| Ab101H12/Ab102L103 | W103L/S30V, L46Q, | SEQ ID NO: 1014 | SEQ ID NO: 1017 |
| | Y49A | | |
| Ab101H12/Ab102L70 | W103L/Y49A, Y87L | SEQ ID NO: 1014 | SEQ ID NO: 1018 |
| Ab101H12/Ab102L130 | W103L/S30V, Y49A, | SEQ ID NO: 1014 | SEQ ID NO: 1019 |
| | Y87L | | |
| Ab101H89/Ab102L69 | V37S, F100aI/L46Q, | SEQ ID NO: 1015 | SEQ ID NO: 1016 |
| | Y49A | | |
| Ab101H89/Ab102L103 | V37S, F100aI/S30V, | SEQ ID NO: 1015 | SEQ ID NO: 1017 |
| | L46Q, Y49A | | |

### 3-7 Pharmacokinetics of Activated Bivalent IL-12 Fusion Proteins Comprising Amino Acid Modifications at the VH/VL Interface

### 3-7-1 In Vivo Test Using SCID Mice

The pharmacokinetics of VH release variants (Table 12) were assessed in SCID mice. VH release variants (0.04 mg/mL) were administrated at a single intravenous administration of 10 mL/kg. Blood was collected at 5 minutes, 4 hours, 1 day, 2 days, 3 days, 7 days, 14 days, 21 days, and 28 days after administration. The collected blood was centrifuged immediately at 14000 rpm at 4 degrees C. for 10 minutes to separate the plasma. The separated plasma was stored at below −20 degrees C. until measurement.

### 3-7-2 Measurement of VH Release Variants in SCID Mice Plasma by ELISA

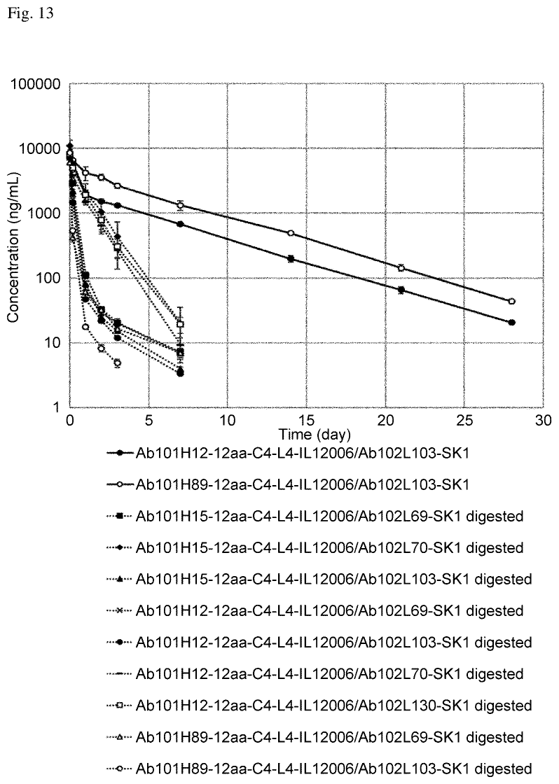

The concentrations of VH release variants in SCID plasma were measured by IL-12 High Sensitivity Human ELISA kit (Abeam) according to the manufacturer's instruction. Concentrations of VH release variants were calculated based on the response of the calibration curve using the analytical software SOFTmax PRO (Molecular Devices). The time course of plasma VH release variants concentrations measured by this method is shown in FIG. 13.

### 3-7-3 Pharmacokinetics VH Release Variants in SCID Mice

Pharmacokinetic profile of VH release variants in SCID mice was evaluated. FIG. 13 illustrates the time course of plasma concentrations of VH release variants after intravenous administration in SCID mice. The clearance of uncleaved bivalent IL-12 fusion proteins, Ab101H12/Ab102L103 and Ab101H89/Ab102L103was 26.6 and 13.9 mL/day/kg, respectively. Compared with uncleaved fusion proteins, all VH release variants had higher clearance levels, particularly Ab101H89/Ab102L69 digested and Ab101H89/Ab102L103 digested had the highest clearance among these variants, with 599 and 615 mL/day/kg, respectively (Table 13, FIG. 13). These results indicate that the VH release variants promoted VH dissociation from the fusion protein activated via protease digestion of its cleavable linker. This exposes the two heparin binding sites on the IL-12 which were otherwise masked when it is bound by the ligand-binding domain of the fusion protein. As a result of the amino acid modifications performed to promote VH dissociation, there is quick uptake to extracellular matrix resulting in rapid elimination.

TABLE 13
Pharmacokinetic parameters (ml/day/kg) of activated bivalent IL-12 fusion
proteins comprising amino acid modifications at VH/VL interface

| | | | AUC_{inf} | CL | Vₛₛ | t_{1/2} | C₀ |
|---|---|---|---|---|---|---|---|
| Variant Name | VH/VL mutations | | day*ng/mL | mL/day/kg | mL/kg | day | ng/mL |
| Ab101H/Ab102L | —/— | Mean | 27368 | 14.8 | 132 | 5.37 | 10215 |
| | | SD | 4083 | 2.2 | 9 | 0.25 | 893 |
| Ab101H12/ | W103L/S30V, | Mean | 15051 | 26.6 | 137 | 4.31 | 8137 |
| Ab102L103 | L46Q, Y49A | SD | 383 | 0.7 | 1 | 0.18 | 139 |
| Ab101H89/ | V37S, F100al/ | Mean | 28892 | 13.9 | 80.1 | 4.03 | 8324 |
| Ab102L103 | S30V, L46Q, Y49A | SD | 1993 | 1 | 7.9 | 0.2 | 332 |
| Ab101H15/ | W103M/L46Q, | Mean | 1696 | 236 | 137 | 2.38 | 7698 |
| Ab102L69 digested | Y49A | SD | 31 | 4 | 12 | 0.07 | 445 |
| Ab101H15/ | W103M/S30V, | Mean | 1481 | 271 | 116 | 1.93 | 9671 |
| Ab102L103 digested | L46Q, Y49A | SD | 72 | 13 | 5 | 0.06 | 392 |
| Ab101H15/ | W103M/Y49A, | Mean | 7134 | 59.8 | 61.6 | 0.86 | 11049 |
| Ab102L70 digested | Y87L | SD | 2368 | 17 | 8.4 | 0.05 | 2588 |
| Ab101H12/ | W103L/L46Q, | Mean | 1313 | 305 | 189 | 2.44 | 8199 |
| Ab102L69 digested | Y49A | SD | 75 | 17 | 18 | 0.23 | 325 |
| Ab101H12/ | W103L/S30V, | Mean | 1039 | 385 | 175 | 1.93 | 7412 |
| Ab102L103 digested | L46Q, Y49A | SD | 36 | 13 | 11 | 0.08 | 102 |
| Ab101H12/ | W103L/Y49A, | Mean | 4677 | 85.6 | 90.1 | 0.817 | 6840 |
| Ab102L70 digested | Y87L | SD | 194 | 3.6 | 7.6 | 0.029 | 296 |
| Ab101H12/ | W103L/S30V, | Mean | 5946 | 68 | 72.2 | 0.932 | 8176 |
| Ab102L130 digested | Y49A, Y87L | SD | 771 | 8.6 | 2.6 | 0.038 | 270 |
| Ab101H89/ | V37S, F100al/ | Mean | 671 | 599 | 764 | 3.17 | 6469 |
| Ab102L69 digested | L46Q, Y49A | SD | 53 | 50 | 316 | 1.68 | 152 |
| Ab101H89/ | V37S, F100al/ | Mean | 654 | 615 | 132 | 1.07 | 9019 |
| Ab102L103 digested | S30V, L46Q, Y49A | SD | 54 | 52 | 11 | 0.08 | 1119 |
| Ab101H/Ab102L | —/— | Mean | 29102 | 13.8 | 67 | 3.44 | 9871 |
| digested | | SD | 2656 | 1.3 | 5 | 0.25 | 246 |

### 3-8 Preparation of Bivalent CXCL10 Fusion Proteins

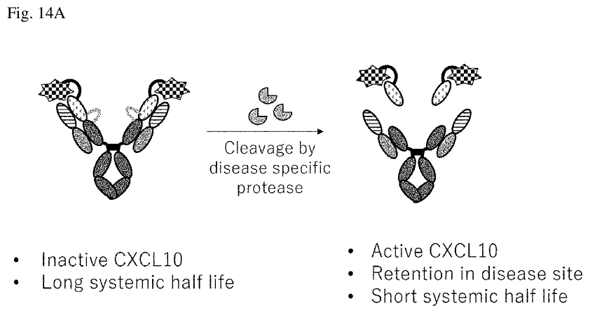

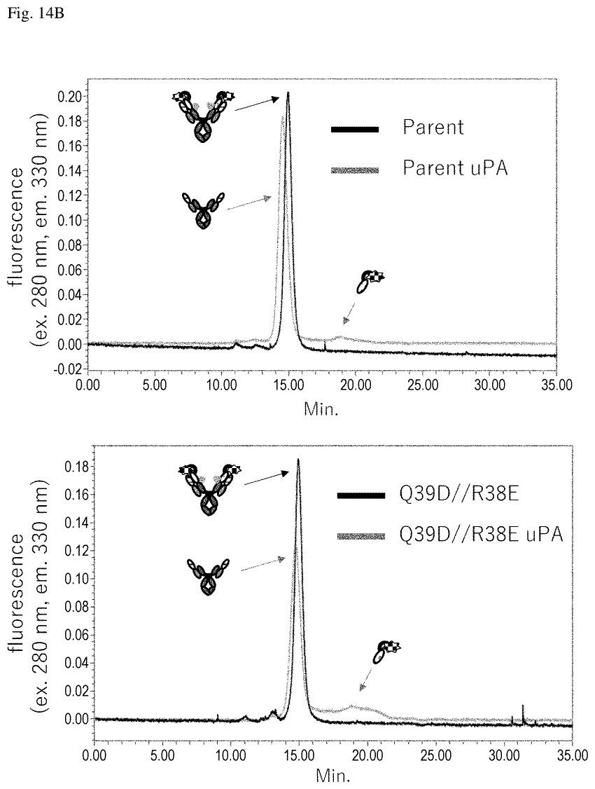

The effectiveness of VH release mutation was also verified with another antibody-antigen combination, CXCL10 and anti-CXCL10 antibody. Similar to IL-12, as reported in literature, CXCL10 has a very short half-life (9.83 h; http://www.ectrx.org/detail/current/2020/18/3/0/368/0). As described above, in the uncleaved (“inactive”) state, the bivalent CXCL10 fusion protein would have a longer half-life due to the masking of GAG binding site when CXCL10 is bound to the antigen-binding domain of an anti-CXCL10 antibody. Upon activation by protease cleavage, CXCL10 is released from the antigen-binding domain, and the cleaved (“active”) state molecule is rapidly eliminated (FIG. 14A).

Bivalent CXCL10 fusion proteins were constructed by fusing CXCL10 molecules (hCXCL10R75A.0041, SEQ ID NO: 1020) with anti-CXCL10 antibodies via cleavable linkers. Unless otherwise noted, Fc region is a modified IgG1 region which contains mutations (L235R/G236R/A327G/A330S/P331S in EU numbering) to abolish Fc gamma R binding.

Bivalent CXCL10 fusion protein FP9 (hCXCL10R75A.0041-L7-HFR0039H-12aa0054-C7/L-LT0) is a homodimer made up of a light chain (SEQ ID NO: 1021) and heavy chain (SEQ ID NO: 1022). In heavy chain (SEQ ID NO: 1022), hCXCL10R75A.0041 is fused to VH region (HFR0039H, SEQ ID NO: 1024) via a GS linker (L7, SEQ ID NO: 1025) and VH region is fused to constant region (C7, SEQ ID NO: 1026) via a cleavable linker (SEQ ID NO: 1027). SEQ ID NO: 1023 was employed as light chain and SEQ ID NO: 1028 was employed as heavy chain with further modifications in the VH/VL interface as described in Table 15.

Expression vectors of each chain were prepared by a method known to those skilled in the art and expressed using Expi293 (Life Technologies Corp.) by combining each chain as shown in Table 14. Purification of fusion protein was done using affinity purification by MABSELECT SURE LX (Cat #: 17547402, GE Healthcare,).

TABLE 14
Bivalent CXCL10 fusion protein and sequence IDs of each chain.

| CXCL10 | Light | Heavy | Light | Heavy |
|---|---|---|---|---|
| fusion protein | chain 1 | chain 1 | chain 2 | chain 2 |
| Bivalent | SEQ ID | SEQ ID | — | — |
| CXCL10 | NO: 1021 | NO: 1022 | | |
| fusion protein | | | | |
| FP9 | | | | |

### 3-9 Screening of VH Release Amino Acid Modification Using Bivalent CXCL10 Fusion Protein

The percentage of VH released from CXCL10 fusion protein was evaluated using two methods, (i) size exclusion chromatography (SEC) and (ii) surface plasmon resonance (SPR).
(i) SEC methods: CXCL10 fusion proteins were applied to TSKgel G3000SWXL column (Cat #: 000854, Tosoh) and separated by using modified mobile phase (50 mM NaPB, 750 mM Arginine-HCl). Peaks were detected by fluorescence (ex. 280 nm, em. 330 nm). For protease treated samples, 0.18 mg/mL CXCL10 fusion proteins were treated with 70 nM uPA (cat. no: 755304, Biolegend) for 1.5 hours. Although the percentage of VH release cannot be calculated from fluorescence peak area, it was observed that charge mutations in the VH/VL interface increase the tendency of ligand release compared to no modifications (FIG. 14B).
(ii) Biacore method: VH release for CXCL10 fusion proteins were determined using Biacore T200 instrument (GE Healthcare). Sure Protein A (Cat #28-4018-60, GE Healthcare) was immobilised onto all flow cells (FCs) of a CM3 sensor chip (Cat #: BR100536, GE Healthcare). All proteins and analytes were prepared in modified running buffer (pH7.4/10 mM HEPES/500 mM NaCl/0.005% SurfactantP20/3 mM EDTA). Each protein was captured onto the sensor surface by Protein A captured at the flow cells. This is followed injection of 200 nM uPA (Cat #: 755304, Biolegend) or buffer across all flow cells. Sensor surface was regenerated after each cycle. The RU value at 5 seconds before the sample injection onto flow cells ended was adopted as the final response for each protein. The percentage reduction in RU was calculated using the following formula:

$$\frac{\begin{matrix}Final response of buffer injection - \\ Final response of uPA injection\end{matrix}}{Final response of buffer injectíon} \times 100$$

As CXCL10-VH corresponds to 30% of the molecular weight of IgG molecule, 30% reduction in response implies 100% CXCL10-VH region has been released.

$$VH release \% = \% reduction in RU \times 100 / 30$$

Charge mutations in the VH/VL interface led to increase CXCL10-VH release tendency compared to no modifications (Table 15).

TABLE 15
CXCL10 fused antibodies with VH release mutations,
sequence IDs of each chain and the VH release %

| Variant | VH/VL | Heavy | Light | VH release % |
|---|---|---|---|---|
| name | mutation | chain | Chain | (Biacore) |
| FP9 | —/— | SEQ ID | SEQ ID | 16% |
| | | NO: 1022 | NO: 1021 | |
| FP9 | Q39D/R38E | SEQ ID | SEQ ID | 55% |
| Q39D/R38E | | NO: 1028 | NO: 1023 | |

Engineering performed at the VH/VL interface via modification of the amino acids residing at the interface between VH and VL reduced the association between VH and VL, promoting the dissociation of VH from the bivalent fusion proteins of the present invention. As examples, fusion proteins comprising two molecular formats and two ligands/antigens were evaluated, i.e. bivalent IL-12 fusion protein and bivalent CXCL10 fusion protein. In both examples, amino acid modifications at the interface between VH and VL promoted the dissociation of VH from the bivalent fusion proteins independent of the molecular format or ligand/antigen identity. In each case, the release of VH disrupts the binding of the ligand to the ligand-binding domain or antigen to the antigen-binding domain, allowing them to bind their respective receptors or binding partners and exert their biological activity. Further, the present inventors have also discovered additionally modifying amino acids residing in the CDR regions were also effective to promote the VH dissociation and the subsequent release of the ligand/antigen from the fusion protein without any significantly changes to the binding kinetics of the ligand/antigen with its ligand-binding domain or antigen-binding domain. In fact, a combination of modifications residing at the interface between VH and VL in the framework regions and the CDR regions demonstrated superior VH release tendency compared to modifications performed in the framework regions alone (Ab101H17/Ab102L69 demonstrated 28.1% VH release vs Ab101H89/Ab102L69 which demonstrated 56% VH release).

### 3-10 Preparation of Bivalent IL-22 Fusion Proteins

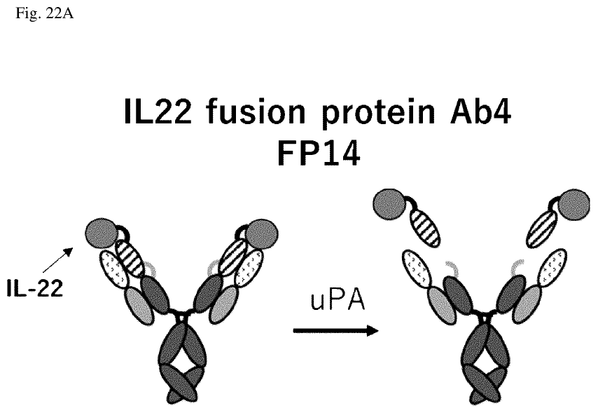

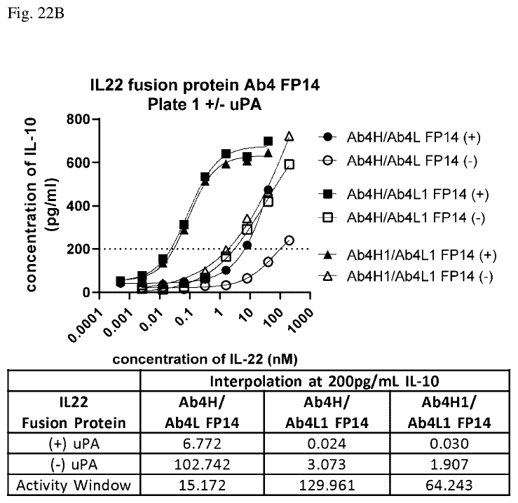

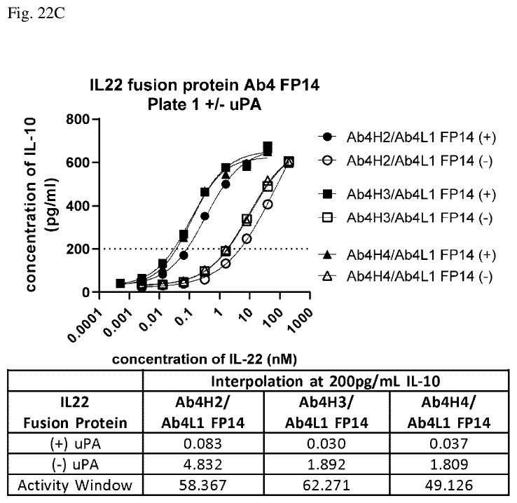

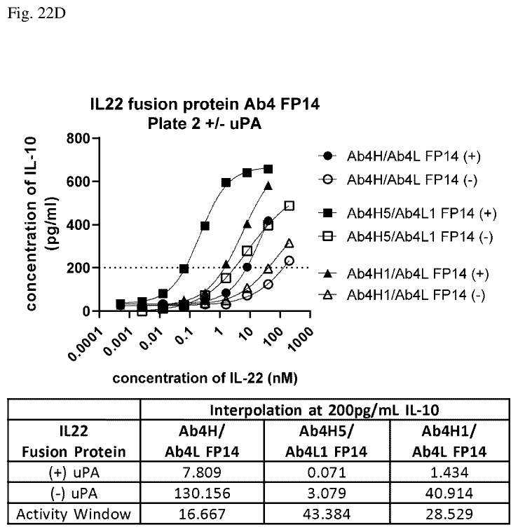

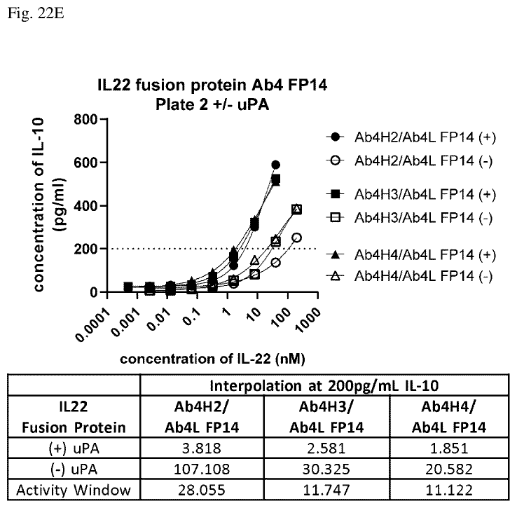

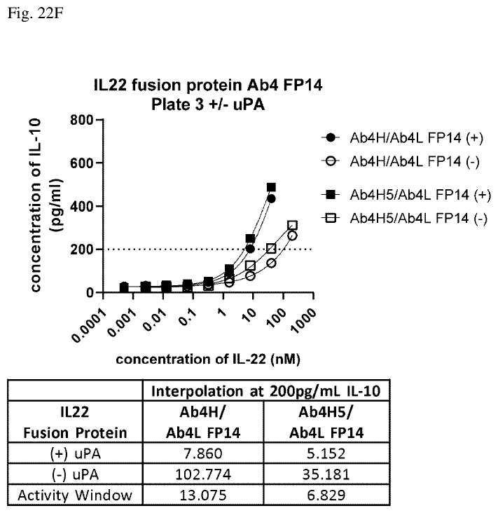

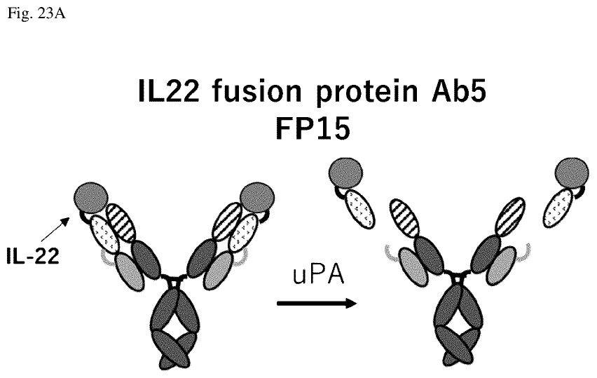

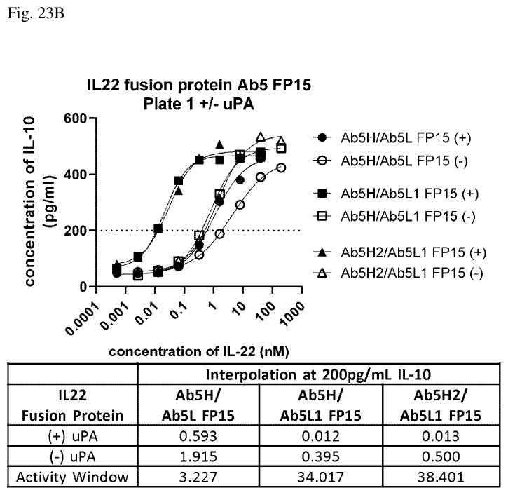

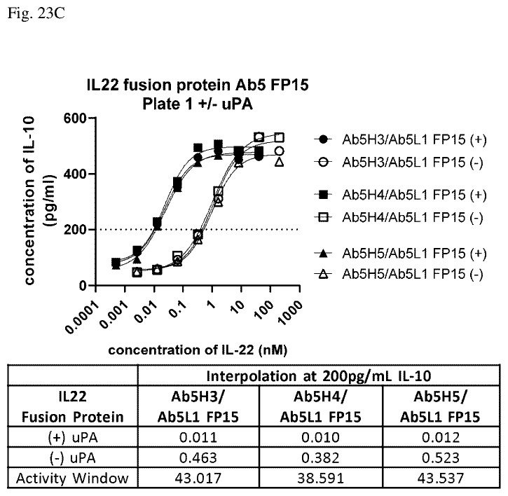

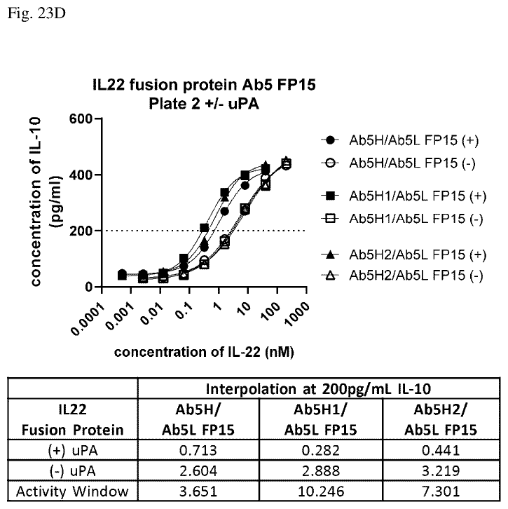

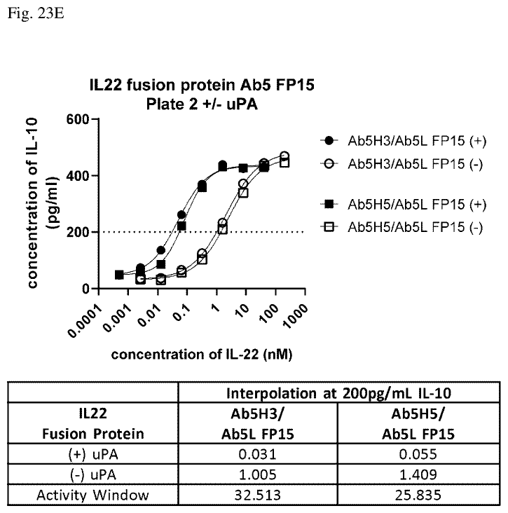

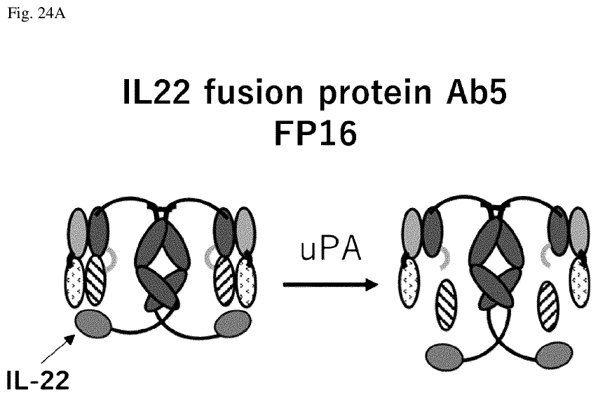

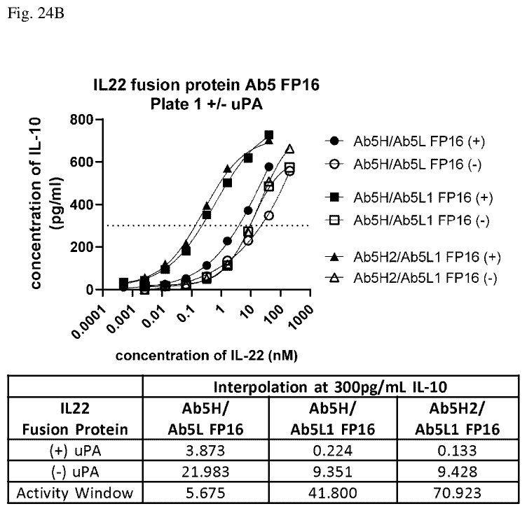

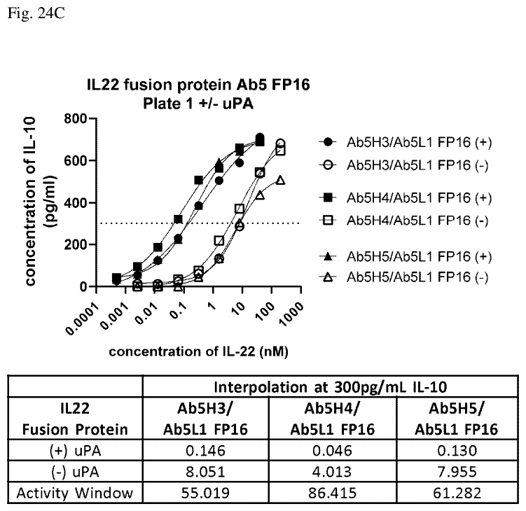

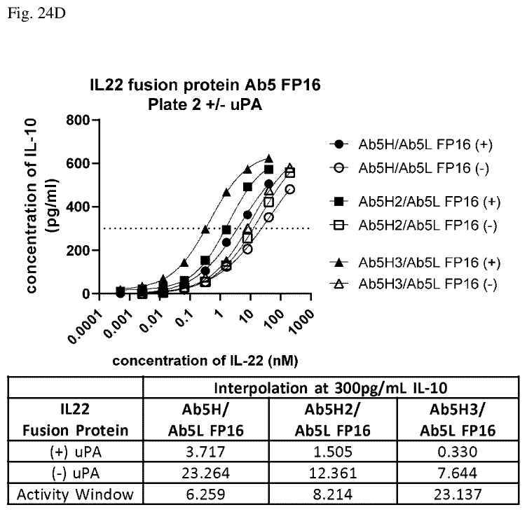

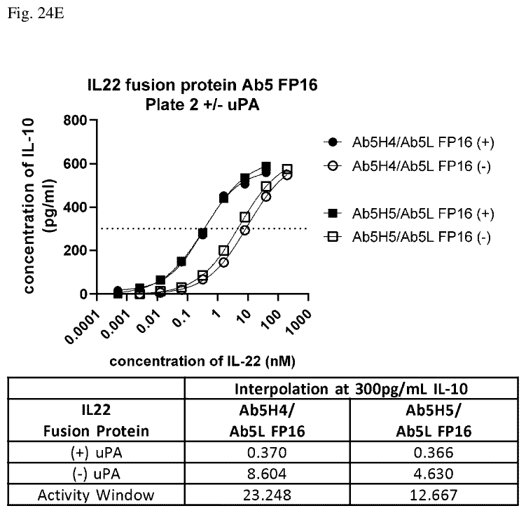

Three IL-22 fusion proteins FP14, FP15 and FP16 were designed, each in different molecular format (FIGS. 22A, 23A and 24A) As shown in FIG. 22A, upon protease cleavage, VH-IL-22 is released (“VH-ligand release”) from FP14, in FIG. 23A, VL-IL-22 is released (“VL-ligand release”) from FP15 and in FIG. 24A, VH is released (“VH-ligand release”) from FP16. Bivalent Interleukin-22 (IL-22) fusion proteins were constructed by fusing IL-22 molecules (SEQ ID NO: 971) with anti-IL-22 antibodies via cleavable linkers. Bivalent IL-22 fusion protein FP14 (IL22-GS-Ab4H-12aa-C6/Ab4L-LT0) is a homodimer made up of a heavy chain (SEQ ID NO: 1095) and a light chain (SEQ ID NO: 1096). In the heavy chain (SEQ ID NO: 1095), IL-22 (SEQ ID NO: 971) is fused to the N-terminus of VH, Ab4H (SEQ ID NO: 1091) via GS linker (SEQ ID NO: 1001). Ab4H (SEQ ID NO: 1091) is fused to the N-terminus of constant region, C6 (SEQ ID NO: 1006) via a cleavable linker, 12aa (SEQ ID NO: 941). The heavy chain (SEQ ID NO: 1095) and light chain (SEQ ID NO: 1096) were further modified at the VH/VL interface by performing at least one amino acid modification at the VH/VL interface. Ab4L is the VL (SEQ ID NO: 1092).

Bivalent IL-22 fusion protein FP15 (Ab5H-C6/IL22-GS-Ab5L-12aa-LT0) is a homodimer made up of a heavy chain (SEQ ID NO: 1097) and a light chain (SEQ ID NO: 1098). In the light chain (SEQ ID NO: 1098), IL-22 (SEQ ID NO: 971) is fused to the N-terminus of VL, Ab5L (SEQ ID NO: 1094) via GS linker (SEQ ID NO: 1001). Ab5L (SEQ ID NO: 1094) is fused to the N-terminus of constant region, C9 (SEQ ID NO: 1101) via a cleavable linker, 12aa (SEQ ID NO: 941). The heavy chain (SEQ ID NO: 1097) and light chain (SEQ ID NO: 1098) were further modified at the VH/VL interface by performing at least one amino acid modification at the VH/VL interface.

Bivalent IL-22 fusion protein FP16 (Ab5H-12aa-C4-L4-IL22/Ab5L-LT0) is a homodimer made up of a heavy chain (SEQ ID NO: 1099) and a light chain (SEQ ID NO: 1100). In the heavy chain (SEQ ID NO: 1099), VH, Ab5H (SEQ ID NO: 1093) is fused to the N-terminus of constant region, C4 (SEQ ID NO: 970) via cleavable linker, 12aa (SEQ ID NO: 941). C4 is connected to IL-22 (SEQ ID NO: 971) via GS linker, L4 (SEQ ID NO: 963). The heavy chain (SEQ ID NO: 1099) and light chain (SEQ ID NO: 1100) were further modified at the VH/VL interface by performing at least one amino acid modification at the VH/VL interface.

Several fusion proteins comprising of FP14, FP15 and FP16 and modifications in the VH/VL interface (“Single mutants” or “Combination mutants”) were examined as shown in Tables 17A, 17B and 17C, respectively.

Expression vectors of each chain were prepared by a method known to those skilled in the art and expressed using Expi293 (Life Technologies Corp.) by combining each chain as shown in Table 16. Purification of fusion proteins (FP) 14, 15 and 16 were done using affinity purification by MabSelect SuRe (Cat. No: 17-5438-01, GE Healthcare) followed by size exclusion chromatography using Superdex 200 gel filtration column (Cat. No: 28-9893-35, GE Healthcare). Any aggregates present in the elution from affinity chromatography were removed using size exclusion chromatography.

TABLE 16
Bivalent IL-22 fusion proteins FP14, FP15
and FP16 and sequence IDs of each chain.

| IL-12 fusion | Light | Heavy | Light | Heavy |
|---|---|---|---|---|
| protein | chain 1 | chain 1 | chain 2 | chain 2 |
| Bivalent IL-22 | SEQ ID | SEQ ID | — | — |
| fusion protein | NO: 1095 | NO: 1096 | | |
| FP14 | | | | |
| Bivalent IL-22 | SEQ ID | SEQ ID | | |
| fusion protein | NO: 1097 | NO: 1098 | | |
| FP15 | | | | |
| Bivalent IL-22 | SEQ ID | SEQ ID | | |
| fusion protein | NO: 1099 | NO: 1100 | | |
| FP16 | | | | |

### 3-11 Screening of VH, VH-Ligand or VL-Ligand Release Amino Acid Modifications Using IL-22 Fusion Protein (FP14, 15 and 16) Variants

The percentage of VH-ligand or VL-ligand released from IL-22 fusion protein FP14 and FP15 respectively was evaluated. VH-ligand or VL-ligand release for bivalent IL-22 fusion proteins was determined at 37 degrees C. using Biacore T200 instrument (Cytiva). Protein A/G (PIERCE) was immobilized onto all flow cells (FCs) of a CM4 sensor chip using amine coupling kit (Cytiva). All proteins and analytes were prepared in HBS-EP+buffer. Each protein was captured onto the sensor surface by Protein A/G captured at flow cells—FC2, FC3, or FC4 to a level of 500 RU, and then followed by 1800 seconds injection of 400 nM of recombinant human uPA or buffer across all FCs. Sensor surface was regenerated after each cycle with 10 mM Glycine-HCl, pH 1.5. The RU value at 10 seconds before the sample injection onto flow cells—FC2, FC3, or FC4 ended was adopted as the final response for each antibody. The percentage reduction in RU was calculated using the following formula:

$$\frac{\begin{matrix}Final response of buffer injection - \\ Final response of uPA injection\end{matrix}}{Final response of buffer injectíon} \times 100$$

As IL-22-VH (“VH-ligand”) corresponds to 36.8% of the molecular weight of the molecular weight of IL22 fusion protein, 36.8% reduction in response implies that 100% of IL-22-VH has been released.

$$VH release \% = \% reduction in RU \times 100 / 36.8$$

As IL-22-VL (“VL-ligand”) corresponds to 36.8% of the molecular weight of molecular weight of IgG molecule, 36.8% reduction in response implies that 100% of IL-22-VL has been released.

$$VH release \% = \% reduction in RU \times 100 / 36.8$$

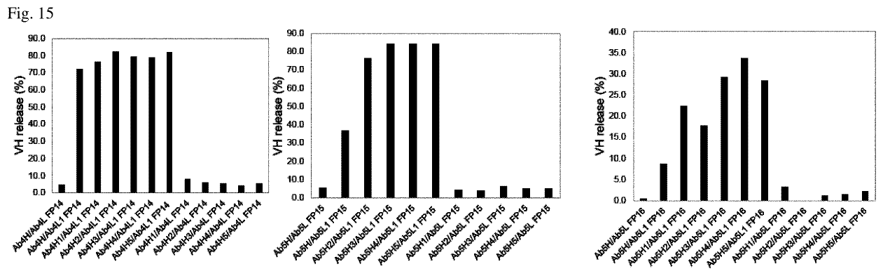

As shown in Tables 17A and 17B, and FIG. 15, all tested variants demonstrated improved VH-ligand or VL-ligand release tendency compared to no modifications.

TABLE 17A
Bivalent IL-22 fusion proteins FP14 with VH-ligand
release mutations and the VH-ligand release %

| | | % | VH- |
|---|---|---|---|
| | | reduction | ligand |
| Variant name | VH/VL mutation | in RU | release % |
| Ab4H/Ab4L | —/— | 1.78 | 4.83 |
| Ab4H/Ab4L1 | —/L46Q, Y49A | 26.61 | 72.3 |
| Ab4H1/Ab4L1 | V37S/L46Q, Y49A | 28.14 | 76.5 |
| Ab4H2/Ab4L1 | Y91A/L46Q, Y49A | 30.18 | 82.0 |
| Ab4H3/Ab4L1 | W103L/L46Q, Y49A | 29.12 | 79.1 |
| Ab4H4/Ab4L1 | W103M/L46Q, Y49A | 29.02 | 78.9 |
| Ab4H5/Ab4L1 | W103I/L46Q, Y49A | 30.12 | 81.9 |
| Ab4H1/Ab4L | V37S/— | 2.89 | 7.85 |
| Ab4H2/Ab4L | Y91A/— | 2.24 | 6.09 |
| Ab4H3/Ab4L | W103L/— | 2.02 | 5.48 |
| Ab4H4/Ab4L | W103M/— | 1.57 | 4.26 |
| Ab4H5/Ab4L | W103I/— | 1.99 | 5.41 |

TABLE 17B
Bivalent IL-22 fusion proteins FP15 with VL
release mutations and the VL-ligand release %

| | | % | VL- |
|---|---|---|---|
| | | reduction | ligand |
| Variant name | VH/VL mutation | in RU | release % |
| Ab5H/Ab5L | —/— | 2.07 | 5.62 |
| Ab5H/Ab5L1 | —/L46Q, Y49A | 13.61 | 37.0 |
| Ab5H2/Ab5L1 | H91A/L46Q, Y49A | 28.08 | 76.3 |
| Ab5H3/Ab5L1 | W103L/L46Q, Y49A | 31.04 | 84.4 |
| Ab5H4/Ab5L1 | W103M/L46Q, Y49A | 31.09 | 84.5 |
| Ab5H5/Ab5L1 | W103I/L46Q, Y49A | 31.04 | 84.3 |
| Ab5H1/Ab5L | V37S/— | 1.60 | 4.34 |
| Ab5H2/Ab5L | H91A/— | 1.38 | 3.74 |
| Ab5H3/Ab5L | W103L/— | 2.29 | 6.23 |
| Ab5H4/Ab5L | W103M/— | 1.91 | 5.20 |
| Ab5H5/Ab5L | W103I/— | 1.90 | 5.17 |

The percentage of VH released from IL-22 fusion protein FP16 respectively was evaluated. VH release for bivalent IL-22 fusion proteins was determined at 37 degrees C. using Biacore T200 instrument (Cytiva). Protein A/G (PIERCE) was immobilized onto all flow cells (FCs) of a CM4 sensor chip using amine coupling kit (Cytiva). All proteins and analytes were prepared in HBS-EP+buffer. Each protein was captured onto the sensor surface by Protein A/G captured at flow cells—FC2, FC3, or FC4 to a level of 500 RU, and then followed by 1800 seconds injection of 400 nM of recombinant human uPA or buffer across all FCs. Sensor surface was regenerated after each cycle with 10 mM Glycine-HCl, pH 1.5. The RU value at 10 seconds before the sample injection onto flow cells—FC2, FC3, or FC4 ended was adopted as the final response for each antibody. The percentage reduction in RU was calculated using the following formula:

$$\frac{\begin{matrix}Final response of buffer injection - \\ Final response of uPA injection\end{matrix}}{Final response of buffer injectíon} \times 100$$

As VH corresponds to 15.8% of the molecular weight of bivalent IL-22 fusion proteins, 15.8% reduction in response implies that 100% of VH has been released.

$$VH release \% = \% reduction in RU \times 100 / 15.8$$

As shown in Table 17C, and FIG. 15, all tested variants demonstrated improved VH release tendency.

TABLE 17C
Bivalent IL-22 fusion proteins FP16 with
VH release mutations and the VH release %

| | | % | |
|---|---|---|---|
| | | reduction | VH |
| Variant name | VH/VL mutation | in RU | release % |
| Ab5H/Ab5L | —/— | 0.07 | 0.46 |
| Ab5H/Ab5L1 | —/L46Q, Y49A | 1.38 | 8.73 |
| Ab5H1/Ab5L1 | V37S/L46Q, Y49A | 3.55 | 22.45 |
| Ab5H2/Ab5L1 | H91A/L46Q, Y49A | 2.80 | 17.71 |
| Ab5H3/Ab5L1 | W103L/L46Q, Y49A | 4.63 | 29.32 |
| Ab5H4/Ab5L1 | W103M/L46Q, Y49A | 5.32 | 33.67 |
| Ab5H5/Ab5L1 | W103I/L46Q, Y49A | 4.48 | 28.35 |
| Ab5H1/Ab5L | V37S/— | 0.50 | 3.15 |
| Ab5H3/Ab5L | W103L/— | 0.18 | 1.12 |
| Ab5H4/Ab5L | W103M/— | 0.23 | 1.48 |
| Ab5H5/Ab5L | W103I/— | 0.34 | 2.16 |

In addition to the ligands IL-12 and CXCL10, IL-22 bivalent fusion proteins were generated in three different molecular formats, VH-IL-22 release (“VH-ligand release”) exemplified with FP14, VL-IL-22 release (“VL-ligand release”) exemplified in FP15, and VH release exemplified with FP16. In this example, it is shown that amino acid modifications performed at the VH/VL interface not only promotes VH release, but also VL release from the bivalent fusion proteins, and is independent of ligand/antigen identity. The release of VH or VL allows them to bind their respective receptors or binding partners to exert their biological activity.

### Example 4: Protease Resistant IL-12

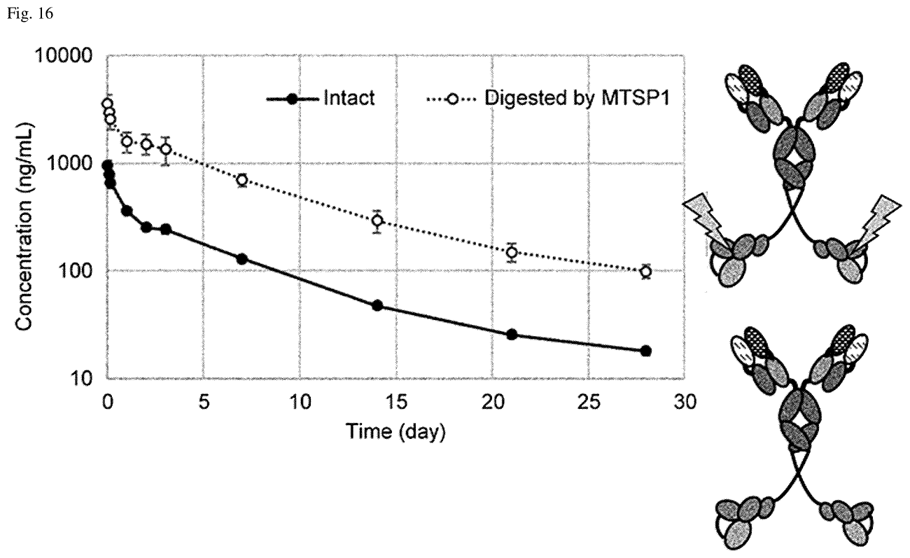

Single-chain IL-12 (SEQ ID NO: 962) is made up of p40 (SEQ ID NO: 939) fused to p35 (SEQ ID NO: 940) by GS linker (SEQ ID: 1029). It is known that the p40 subunit of IL-12 comprises a heparin binding site which is prone to cleavage by tumour specific proteases, especially Human Matriptase/ST14 Catalytic Domain (MT-SP1). Cleavage can occur in the heparin binding region (SEQ ID NO: 1030), between K260 and R261 of p40. Additionally, MT-SP1 cleavage could occur at position Arginine (R) of the N-terminus of p35 (SEQ ID NO: 1031) followed by GS linker (SEQ ID: 1029) within the single chain IL-12 (SEQ ID NO: 962). In the particular case of Bivalent IL-12 fusion protein FP8, the heparin binding site of IL-12 is in close proximity to the epitope of the variable region of IL-12 fusion protein (SEQ ID NO: 1012). Protease cleavage at the heparin binding site affects the fast clearance of activated IL-12 fusion protein of the fusion format (FIG. 16). Selective modifications were performed that prevented unintentional cleavage of IL-12 at the heparin binding site but preserved the epitope of IL-12 fusion protein, maintaining its ability to bind IL-12 in the inactive state of the IL-12 fusion protein. KLH Bivalent IL-12 fusion FP7 was utilised to screen for such modifications.

### 4-1 Preparation of KLH Bivalent IL-12 Fusion Proteins

KLH Bivalent IL-12 fusion FP10 (KLH-Bivalent IL12006v1) is a homodimer made up of a light chain (SEQ ID NO: 986) and heavy chain (SEQ ID NO: 1032). SEQ ID NO 986 was employed as light chain without modifications. In heavy chain (SEQ ID NO: 1032), KLH VH (SEQ ID NO: 994) is fused to constant region (SEQ ID NO: 1006) followed by single-chain IL-12 (SEQ ID NO: 1033) attached to C-terminal of Fc via GS linker (SEQ ID NO: 963). Several other fusion proteins comprising of FP10 (KLH-Bivalent IL12006v1) and IL-12 variants (“IL-12 variants”) were examined as shown in Table 19.

Expression vectors of each chain were prepared by a method known to those skilled in the art and expressed using Expi293 (Life Technologies Corp.) by combining each chain as shown in Table 18. Purification of proteins was done using affinity purification by MabSelect SuRe (Cat. No: 17-5438-01, GE Healthcare) followed by size exclusion chromatography using Superdex 200 gel filtration column (Cat. No: 28-9893-35, GE Healthcare). Any aggregates present in the elution from affinity chromatography were removed using size exclusion chromatography.

TABLE 18
Bivalent KLH fusion protein and sequence IDs of each chain.

| KLH fusion | Light | Heavy | Light | Heavy |
|---|---|---|---|---|
| protein | chain 1 | chain 1 | chain 2 | chain 2 |
| Bivalent KLH | SEQ ID | SEQ ID | — | — |
| fusion protein | NO: 986 | NO: 1032 | | |
| FP10 | | | | |

TABLE 19
KLH-Bivalent IL-12 fusion proteins with protease
resistant IL-12 mutations, IL-12 variants

| | Heavy | Light | IL-12 |
|---|---|---|---|
| Variant name | chain | Chain | mutation |
| KLH-Bivalent | SEQ ID | SEQ ID | — |
| IL12006v1 | NO: 1032 | NO: 986 | |
| KLH-Bivalent | SEQ ID | SEQ ID | KSHRE |
| IL12006v1.KSHRE | NO: 1034 | NO: 986 | |
| KLH-Bivalent | SEQ ID | SEQ ID | KSHHE |
| IL12006v1.KSHHE | NO: 1035 | NO: 986 | |
| KLH-Bivalent | SEQ ID | SEQ ID | KSHKE |
| IL12006v1.KSHKE | NO: 1036 | NO: 986 | |
| KLH-Bivalent | SEQ ID | SEQ ID | KSHSE |
| IL12006v1.KSHSE | NO: 1037 | NO: 986 | |
| KLH-Bivalent | SEQ ID | SEQ ID | KSKHRE |
| IL12006v1.KSKHRE | NO: 1038 | NO: 986 | |
| KLH-Bivalent | SEQ ID | SEQ ID | KSKQRE |
| IL12006v1.KSKQRE | NO: 1039 | NO: 986 | |
| KLH-Bivalent | SEQ ID | SEQ ID | KSKERE |
| IL12006v1.KSKERE | NO: 1040 | NO: 986 | |
| KLH-Bivalent | SEQ ID | SEQ ID | KSKPRE |
| IL12006v1.KSKPRE | NO: 1041 | NO: 986 | |
| KLH-Bivalent | SEQ ID | SEQ ID | KHKE |
| IL12006v1.KHKE | NO: 1042 | NO: 986 | |
| KLH-Bivalent | SEQ ID | SEQ ID | KHHE |
| IL12006v1.KHHE | NO: 1043 | NO: 986 | |
| KLH-Bivalent | SEQ ID | SEQ ID | KHRE |
| IL12006v1.KHRE | NO: 1044 | NO: 986 | |
| KLH-Bivalent | SEQ ID | SEQ ID | KKHE |
| IL12006v1.KKHE | NO: 1045 | NO: 986 | |
| KLH-Bivalent | SEQ ID | SEQ ID | KRHE |
| IL12006v1.KRHE | NO: 1046 | NO: 986 | |
| KLH-Bivalent | SEQ ID | SEQ ID | KRE |
| IL12006v1.KRE | NO: 1047 | NO: 986 | |
| KLH-Bivalent | SEQ ID | SEQ ID | KHE |
| IL12006v1.KHE | NO: 1048 | NO: 986 | |
| KLH-Bivalent | SEQ ID | SEQ ID | KKE |
| IL12006v1.KKE | NO: 1049 | NO: 986 | |

### 4-2 Protease Digestion of KLH-Bivalent IL-12 Fusion Proteins

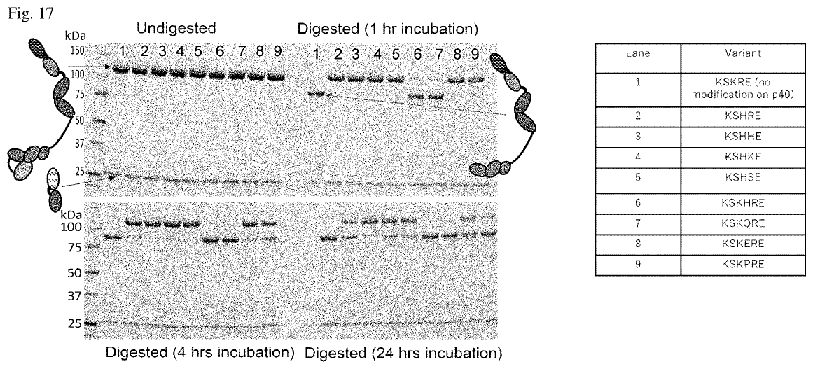

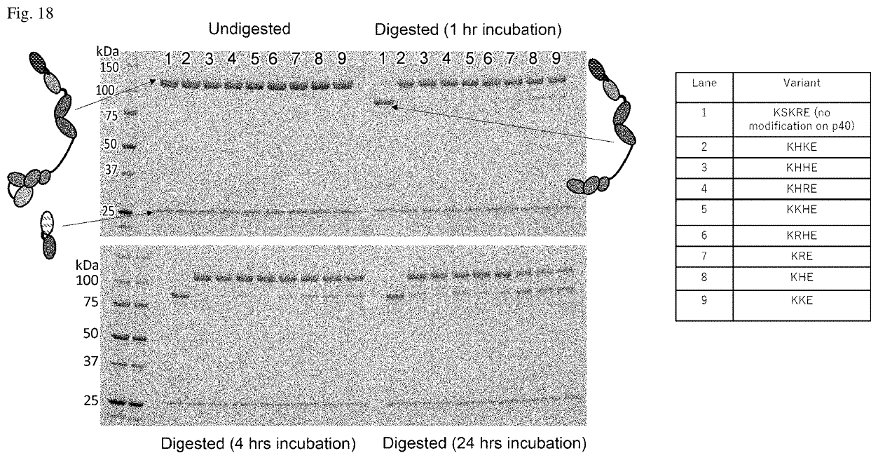

Recombinant Human Matriptase/ST14 Catalytic Domain (MT-SP1) (R&D Systems, Inc., 3946-SE-010) was used as the protease. 75 nM protease and 750 nM of each fusion protein were reacted in PBS under a condition of 37 degrees C. for 1, 4 and 24 hours. Then, cleavage by the protease was evaluated by reducing SDS-PAGE. The results are shown in FIGS. 17 and 18. It is noted that after protease digestion of 1 h and 4h, there is no change in molecular weight of the heavy chain of most of the fusion proteins comprising the mutations listed in Table 19. This means that most IL-12 variants above were stable and resistant to protease digestion unlike the control (Lane 1). While the results after 1 h and 4h digestion is considered representative for the expected half-life of the activated form of the bivalent IL-12 fusion protein of the present invention, as a worst case, protease digestion of the IL-12 variants up to 24h was also performed. As shown, several of these variants (“digested 24 hrs”) still exhibited molecular weights comparable to the start of the incubation (“undigested”) and remain stable despite the long incubation with protease.

4-3 Evaluation of In Vitro Activity of IL-12 Variants with Improved Protease Resistance

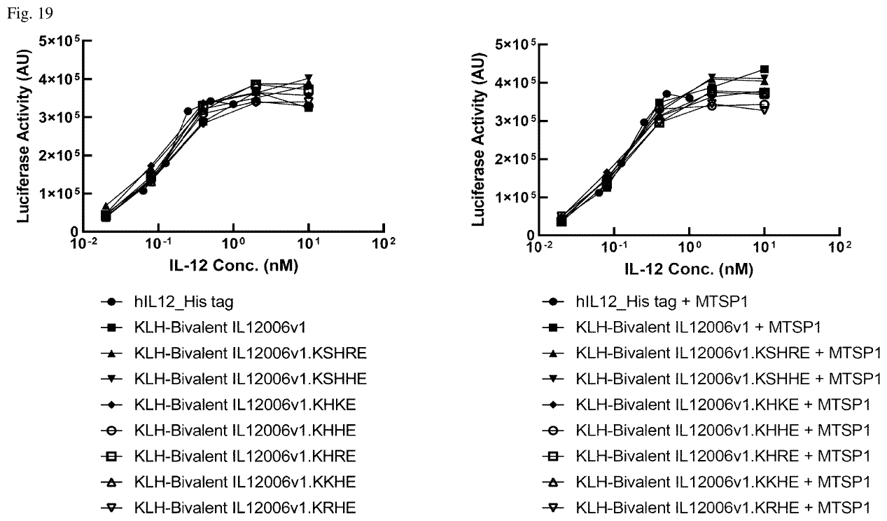

To assess if the protease resistant modifications affect the activity of IL-12 variants with and without protease treatment, IL-12 luciferase assay was conducted. Briefly, 2.5×10⁴ cells/well IL-12 bioassay cell (Promega, Cat #CS2018A02A) which express human IL-12Rb1, IL-12Rb2, and STAT4, were plated in 96-well plate and incubated overnight. Then, IL-12 or KLH-Bivalent IL-12 fusion proteins comprising protease resistant IL-12 (“IL-12 variants”) that were selected from Example 4-2, were added to the culture plate and incubated for 18 hours. The list of fusion proteins are listed in Table 20 below. For protease-treated samples, IL-12 or IL-12 variants were treated with equimolar concentration of MT-SP1 for 4 hours and serial diluents were prepared. Luciferase activity was detected with Bio-Glo luciferase assay system (Promega, G7940) according to manufacturer's instructions. Luminescence was detected using GloMax (registered trademark) Explorer System (Promega #GM3500). Data analysis was done by Microsoft (registered trademark) Excel (registered trademark) 2013 and the analyzed data was plotted using GraphPad Prism 8.4.3. IL-12 activity of protease resistant IL-12 variants was evaluated by the luciferase assay. All the protease resistant IL-12 variants indicated similar activity to hIL12_His tag regardless of protease treatment (FIG. 19).

TABLE 20
Selected IL-12 variants for Pharmacokinetics analysis

| Variant name | Heavy chain | Light Chain | IL12 mutation |
|---|---|---|---|
| KLH-Bivalent IL12006v1 | SEQ ID NO: 1032 | SEQ ID NO: 986 | — |
| KLH-Bivalent IL12006v1.KSHRE | SEQ ID NO: 1034 | SEQ ID NO: 986 | KSHRE |
| KLH-Bivalent IL12006v1.KSHHE | SEQ ID NO: 1035 | SEQ ID NO: 986 | KSHHE |
| KLH-Bivalent IL12006v1.KHKE | SEQ ID NO: 1042 | SEQ ID NO: 986 | KHKE |
| KLH-Bivalent IL12006v1.KHHE | SEQ ID NO: 1043 | SEQ ID NO: 986 | KHHE |
| KLH-Bivalent IL12006v1.KHRE | SEQ ID NO: 1044 | SEQ ID NO: 986 | KHRE |
| KLH-Bivalent IL12006v1.KKHE | SEQ ID NO: 1045 | SEQ ID NO: 986 | KKHE |
| KLH-Bivalent IL12006v1.KRHE | SEQ ID NO: 1046 | SEQ ID NO: 986 | KRHE |

4-4 Evaluation of Pharmacokinetics of IL-12 Variants with Improved Protease Resistance

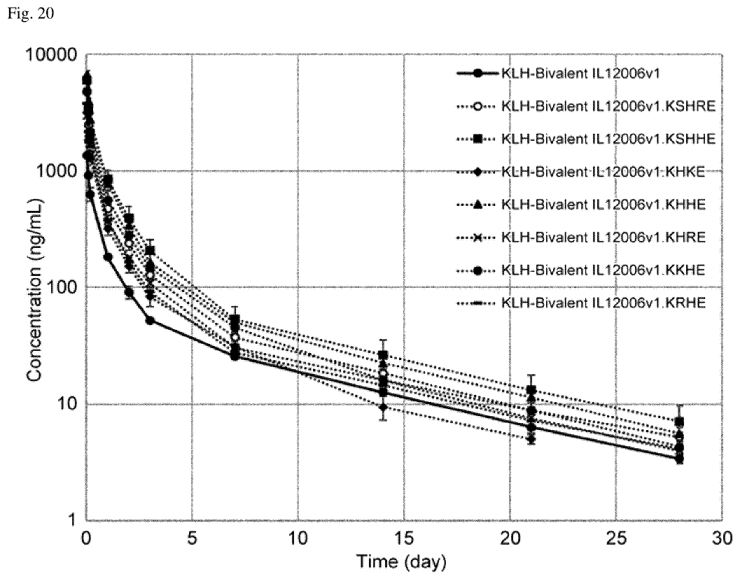

The pharmacokinetics of protease resistant variants were assessed in SCID mice (FIG. 20). Protease resistant variants (0.04 mg/mL) were administrated at a single intravenous administration of 10 mL/kg. Blood was collected at 5 minutes, 2 hours, 4 hours, 1 day, 2 days, 3 days, 7 days, 14 days, 21 days, and 28 days after administration. The collected blood was centrifuged immediately at 14000 rpm at 4 degrees C. for 10 minutes to separate the plasma. The separated plasma was stored at below −20 degrees C. until measurement. The following protease resistant variants shown in Table 20 were tested.

### 4-4-1 Measurement of Protease Resistant Variants in SCID Mice Plasma by ELISA

The concentrations of protease resistant variants in SCID plasma were measured by IL-12 High Sensitivity Human ELISA kit (Abcam) according to the instruction. Protease resistant variants concentrations were calculated based on the response of the calibration curve using the analytical software SOFTmax PRO (Molecular Devices). The time course of plasma protease resistant variants concentrations measured by this method is shown in FIG. 20.

### 4-4-2 Pharmacokinetics Protease Resistant Variants in SCID Mice

Pharmacokinetic profile of protease resistant variants in SCID mice was evaluated. FIG. 20 illustrates the time course of plasma concentrations of protease resistant IL-12 variants after intravenous administration in SCID mice. With reference to FIG. 20 and Table 21, all protease resistant variants demonstrated slower elimination that control (KLH-Bivalent IL12006v1) having a clearance level of 376 mL/day/kg. Amongst these, fusion proteins variants KLH-Bivalent IL12006v1.KHKE and KLH-Bivalent IL12006v1.KRHE showed the highest clearance levels of 241 and 206 mL/day/kg respectively. These variants exemplified a pharmacokinetic profile that demonstrated both protease resistance while retaining high clearance levels.

TABLE 21
Pharmacokinetic parameters (ml/day/kg) of activated bivalent
KLH fusion proteins comprising protease resistant mutations

| | | AUC_{inf} | CL | | | |
|---|---|---|---|---|---|---|
| | | day*ng/ | mL/ | Vₛₛ | t_{1/2} | C₀ |
| Variant Name | | mL | day/kg | mL/kg | day | ng/mL |
| KLH-Bivalent | Mean | 1065 | 376 | 2006 | 7.13 | 1391 |
| IL12006v1 | SD | 43 | 15 | 235 | 0.53 | 231 |
| KLH-Bivalent | Mean | 2511 | 160 | 612 | 7.73 | 4896 |
| IL12006v1.KSHRE | SD | 159 | 10 | 58 | 0.32 | 553 |
| KLH-Bivalent | Mean | 3617 | 112 | 419 | 7.44 | 6158 |
| IL12006v1.KSHHE | SD | 563 | 17 | 61 | 0.86 | 1223 |
| KLH-Bivalent | Mean | 1672 | 241 | 705 | 4.71 | 3228 |
| IL12006v1.KHKE | SD | 176 | 24 | 58 | 0.12 | 215 |
| KLH-Bivalent | Mean | 3646 | 110 | 350 | 6.63 | 6817 |
| IL12006v1.KHHE | SD | 195 | 6 | 53 | 0.21 | 614 |
| KLH-Bivalent | Mean | 1944 | 206 | 806 | 6.87 | 3061 |
| IL12006v1.KHRE | SD | 92 | 10 | 96 | 0.17 | 356 |
| KLH-Bivalent | Mean | 2865 | 140 | 464 | 7.06 | 4940 |
| IL12006v1.KKHE | SD | 216 | 10 | 88 | 0.94 | 583 |
| KLH-Bivalent | Mean | 2003 | 200 | 735 | 7.47 | 3885 |
| IL12006v1.KRHE | SD | 49 | 5 | 25 | 0.43 | 306 |

### Example 5: Biological Activity of IL-12 and IL-22 Fusion Proteins Restored Upon Protease Activation

5-1 Preparation of IL-12 Fusion Proteins with Selected Amino Acid Modifications at VH/VL Interface and Protease Resistant IL-12 Modifications

The ability of IL-12 obtained in Example 4 to bind the ligand-binding domain obtained in Example 3 of the fusion protein of the present invention was evaluated. Bivalent IL-12 fusion protein FP8 (Ab101H-12aa-C4-L4-IL12006/Ab102L-SK1) is a homodimer made up of a light chain (SEQ ID NO: 1009) and heavy chain (SEQ ID NO: 1012). SEQ ID NO: 1009 was employed as light chain with modification(s) in the VH/VL interface. In heavy chain (SEQ ID NO: 1012), VH, Ab101H (SEQ ID NO: 1011) is fused to the N-terminus of constant region, C4 (SEQ ID NO: 970) via a cleavable linker, 12aa (SEQ ID NO: 941) and single chain IL-12, IL12006 (SEQ ID NO: 1008) is fused to the C-terminus of constant region by a GS linker (SEQ ID NO: 963). Ligand-binding domains (Ab101H89/Ab102L69 and Ab101H89/Ab102L103) which have shown improved VH dissociation from the fusion protein after protease cleavage in Example 3 were selected. Single-chain IL-12 variant obtained in Example 4 was attached to C-terminal of Fc domain via GS linker (L4, SEQ ID NO: 963). Several fusion proteins comprising of FP8-12 with/without protease resistant IL-12 variants selected from Example 4 were produced (Table 22).

Expression vectors of each chain were prepared by a method known to those skilled in the art and expressed using Expi293 (Life Technologies Corp.) by combining each chain as shown in Table 22. Purification of proteins was done using affinity purification by MabSelect SuRe (Cat. No: 17-5438-01, GE Healthcare) followed by size exclusion chromatography using Superdex 200 gel filtration column (Cat. No: 28-9893-35, GE Healthcare). Any aggregates present in the elution from affinity chromatography were removed using size exclusion chromatography.

TABLE 22
IL-12 fusion proteins with protease resistant IL-12 variants

| Bivalent IL-12 | Heavy | Light | IL12 |
|---|---|---|---|
| fusion protein | chain | chain | mutation |
| Bivalent IL-12 | SEQ ID | SEQ ID | — |
| fusion protein FP8 | NO: 1012 | NO: 1009 | |
| Bivalent IL-12 | SEQ ID | SEQ ID | KHKE |
| fusion protein | NO: 1050 | NO: 1016 | |
| FP11 | | | |
| Bivalent IL-12 | SEQ ID | SEQ ID | KHKE |
| fusion protein | NO: 1050 | NO: 1017 | |
| FP12 | | | |

5-2 Evaluation of In Vitro Activity of IL-12 Fusion Type Fusion Proteins with Protease Resistant IL-12

To assess if the protease resistant modifications affect the binding of IL-12 to the IL-12 fusion proteins of the present invention, IL-12 luciferase assay was conducted. Briefly, 2.5×10⁴ cells/well IL-12 bioassay cell (Promega, Cat #CS2018A02A) which express human IL-12Rb1, IL-12Rb2, and STAT4, were plated in 96-well plate (#Corning, #3917). Then, the IL-12 fusion proteins were added to the culture plate and incubated for 18 hours. For protease-treated samples, fusion proteins were treated with equimolar concentration of MT-SP1 for 4 hours and serial diluents were prepared. Luciferase activity was detected with Bio-Glo luciferase assay system (Promega, G7940) according to manufacturer's instructions. Luminescence was detected using GloMax (registered trademark) Explorer System (Promega #GM3500). Data analysis was done by Microsoft (registered trademark) Excel (registered trademark) 2013 and the analyzed data was plotted using GraphPad Prism ver. 9.0.2.

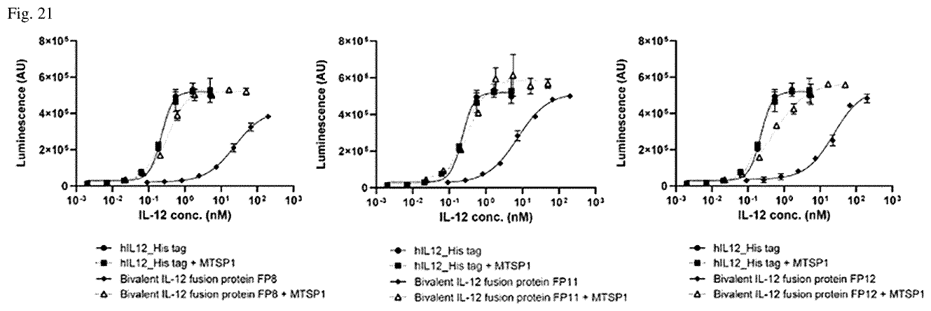

Bivalent IL-12 fusion proteins FP8, FP11 and FP12 were subjected to the IL-12 luciferase assay. All three fusion proteins showed lower IL-12 bioactivity than hIL-12_His tag in the absence of MT-SP1, and the IL-12 bioactivity was restored to the same level as hIL-12_His tag upon MT-SP1 treatment (FIG. 21).

5-3 Evaluation of In Vitro Activity of IL-22 Fusion Proteins with and without Protease Digestion

To assess if the modification of the amino acids residing at the interface between VH and VL promoted the release of IL-22, an activity assay was conducted using COL0205 colon carcinoma cells (Cat No: CCL-222, ATCC) which respond to IL-22 by secreting IL-10 (Int Immunopharmacol. 2004 May; 4(5):679-91).

IL-22 fusion proteins and recombinant human uPA protease were each diluted in serum free RPMI media (Gibco) containing 50 micro M HEPES (Gibco) to 1400 nM concentration, and then mixed in equal volume to arrive at a final concentration of 700 nM antibody and 700 nM uPA protease. For no uPA treatment, fusion protein samples were mixed with serum free RPMI media and HEPES. Samples were incubated for 4 hours at 37 degrees C. in a Thermocycler (2720 Applied Biosystems) to allow for cleavage of the linker and release of IL-22. As a positive control, HEK293-derived recombinant human IL-22 (Cat No: Z03081-50, Genscript) was incubated with uPA.

COL0205 cells were collected by trypsinization with 0.25% trypsin (Gibco), and subsequently filtered through a 70 micrometer cell strainer (Corning) to remove cell clumps. Cell counts were performed using Luna Dual Fluorescence Cell Counter (Logos Biosystem), and 3E4 cells in 50 micro L were seeded into each well of a NUNC Edge 96-well flat bottom plate. Sterile PBS was added to the peripheral wells of the NUNC Edge plate to reduce evaporation from wells along the edges. The cells were incubated for a minimum of 4 hours at 37 degrees C. in a 5% CO2 incubator to allow cells to attach to the plate. IL-22 fusion proteins incubated with and without uPA were serially diluted to 6 times of the final desired concentration, and 10 micro L was added to the cells for a final assay volume of 60 micro L. The assay plate was further incubated overnight at 37 degrees C. in a 5% CO2 incubator. After overnight incubation, the assay plate was centrifuged at 300 g for 3 minutes at 25 degrees C. Cell supernatant samples were collected to quantify the amount of IL-10 produced by COL0205 cells using Human IL-10 DuoSet ELISA (R&D Systems). The procedures for Human IL-10 ELISA were performed according to the manufacturer's recommendation except for the preparation of IL-10 standard and samples. IL-10 standard was diluted in COL0205 culture media, RPMI 1640 medium (Gibco) containing 10% Fetal Bovine Serum (Sigma) and 1% Penicillin-Streptomycin (Gibco), so that the cell supernatant samples could be assayed without dilution for more sensitive IL-10 detection. The absorbance of samples was measured at 450 nm and 595 nm using MultiSkan GO plate reader (Thermo Scientific). Data analysis was done by Microsoft (registered trademark) Excel (registered trademark) and the analyzed data was plotted using GraphPad Prism.

To compare the relative amount of active IL-22 released after protease digestion in each construct, the inventors interpolated the graphs to determine the molar concentration of IL-22 required for the COL0205 cells to secrete a fixed amount of IL-10 in the culture supernatant. In particular, the difference in the amount of active IL-22 released with and without protease was compared between each construct. This is reported as the activity window.

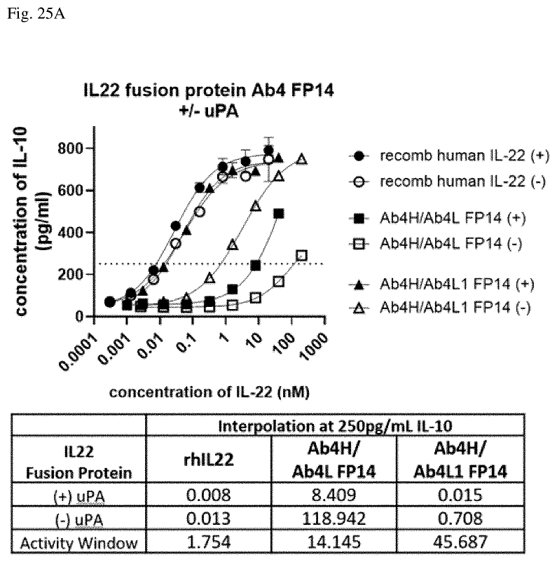

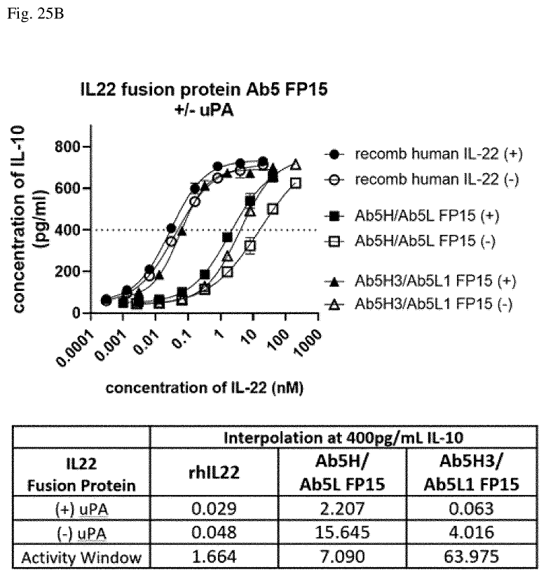

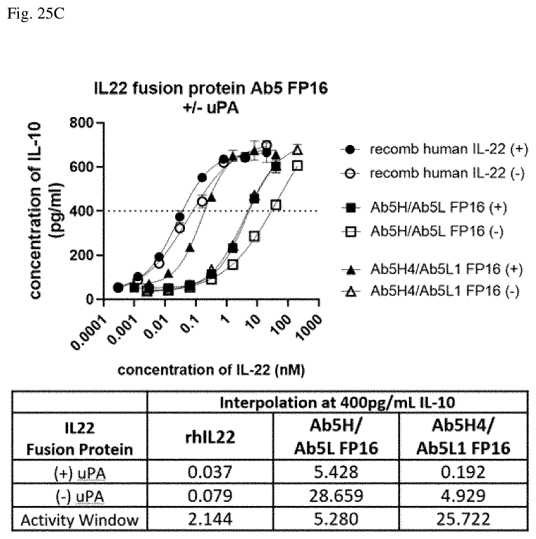

As shown in FIG. 22 (B to F), the activity window (“(−)uPA”/“(+)uPA”) for IL-22 fusion protein FP14 without any mutation (Ab4H/Ab4L) ranged from approximately 13 to 16 across different assay plates. However, the activity window was increased for several constructs carrying mutations in the VH/VL interface. In FIG. 23 (B to E), the activity window for IL-22 fusion protein FP15 without any mutation (Ab4H/Ab4L) was approximately 3 fold across different assay plates, and was increased for several constructs carrying mutations in the VH/VL interface. Similarly, as shown in FIG. 24 (B to E), the same trend of increased activity window of IL-22 fusion protein FP16 was seen when VH/VL interface mutations were applied. In FIGS. 22 to 24, each antibody was evaluated in a single well with no duplicate due to the large panel of antibodies to be evaluated coupled with the requirement that each antibody was evaluated with and without protease. To demonstrate the activity of the released IL-22 relative to recombinant IL-22, the inventors selected one VH/VL interface variant from each fusion protein and compared it against the parental fusion protein without any VH/VL interface mutations, as well as against recombinant IL-22 where each antibody was evaluated in duplicate. As shown in FIG. 25, the activity of the released IL-22 in the selected variants was close to the activity of recombinant IL-22, demonstrating that the modifications at the VH/VL interface can contribute to the activity of the released ligand.

Bivalent IL-22 fusion proteins FP14, FP15 and FP16 were subjected to an activity assay as described above. All of the tested fusion proteins showed lower IL-22 bioactivity in the absence of uPA, and the IL-22 bioactivity was increased upon uPA digestion.

The present inventors have successfully produced bivalent fusion proteins that are activable by specific protease cleavage, having a long half-life when inactive and short half-life when active.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### SEQUENCE LISTING

### C1-A2114Psq.xml

1. A polypeptide comprising at least one antigen-binding domain comprising a protease cleavage site, whereupon cleavage at the protease cleavage site, an antibody domain adjacent to the protease cleavage site dissociates and wherein the dissociation is promoted by at least one amino acid modification performed at the interface between said antibody domain and a corresponding interacting domain, wherein the polypeptide is an antibody that is monovalent or bivalent, monospecific or bispecific, or an IgG antibody selected from the group consisting of IgG1, IgG2, IgG3, IgG4, IgG-IgG, IgG-Fab, or CrossMab antibody; or wherein the polypeptide is an antibody fragment, the antibody fragment is selected from the group consisting of scFv, scFv-Fc, tandem scFv, Fab, tandem Fab, F(ab′)₂, Fab₂, Fab-scFv-Fc, F(ab′)₂-scFv₂, bispecific Fab₂, trispecific Fab₂, bispecific diabody, trispecific diabody, tandem diabody, triabody, tetrabody, minibody, bibody or tribody.

2.-17. (canceled)
